SEQUENCE LISTING (1 sequence)

SEQ ID NO: 1 - protein, 6 aa, synthetic construct
  MOD_RES 1: note = N-(2-furoyl)leucine
  MOD_RES 6: note = AMIDATION
  MOD_RES 6: note = Orn
LIGRLX

(19) United States
(12) Patent Application Publication
(10) Pub. No.: US 2025/0320224 A1
(43) Pub. Date: Oct. 16, 2025

(54) NOVEL PAR-2 INHIBITORS

(71) Applicant: DOMAIN THERAPEUTICS, Illkirch-Graffenstaden (FR)

(72) Inventors: Baptiste RUGERI, Illkirch-Graffenstaden (FR); Florence CHERY, llkirch-Graffenstaden (FR); Gaël HOMMET, Illkirch-Graffenstaden (FR); Stanislas MAYER, Illkirch-Graffenstaden (FR); Stephan SCHANN, Illkirch-Graffenstaden (FR)

(73) Assignee: DOMAIN THERAPEUTICS, Illkirch-Graffenstaden (FR)

(21) Appl. No.: 18/868,129

(22) PCT Filed: Jun. 2, 2023
(86) PCT No.: PCT/EP2023/064898

§ 371 Date: Nov. 21, 2024

(30) Foreign Application Priority Data
Jun. 3, 2022 (EP) 22305814.0

(51) Int. Cl. C07D 491/20; A61K 31/437; A61K 31/438; A61K 31/439; A61K 31/444; A61K 31/4545; A61K 31/496; A61K 31/4985; A61K 31/506; A61K 31/519; A61K 31/5377; A61K 31/5383; A61K 31/551; A61K 31/553; C07D 471/04; C07D 471/10; C07D 487/04; C07D 491/147; C07D 498/04; C07D 519/00

(52) U.S. Cl. CPC C07D 491/20; A61K 31/437; A61K 31/438; A61K 31/439; A61K 31/444; A61K 31/4545; A61K 31/496; A61K 31/4985; A61K 31/506; A61K 31/519; A61K 31/5377; A61K 31/5383; A61K 31/551; A61K 31/553; C07D 471/04; C07D 471/10; C07D 487/04; C07D 491/147; C07D 498/04; C07D 519/00

(57) ABSTRACT

The present invention provides novel compounds of formula (I) and pharmaceutical compositions containing these compounds. The compounds of formula (I) can act as PAR-2 inhibitors, which renders these compounds highly advantageous for use in therapy, particularly in the treatment or prevention of pain, an autoimmune disorder, an autoinflammatory disorder, an inflammatory disorder, a central nervous system disorder, spinal cord injury, a metabolic disorder, a gastrointestinal disorder, a cardiovascular disorder, a fibrotic disorder, a respiratory disorder, a skin disorder, an allergic disorder, or cancer (I).

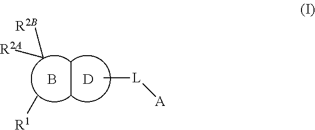

The present invention provides novel compounds of formula (I) and pharmaceutical compositions containing these compounds. The compounds of formula (I) can act as PAR-2 inhibitors, which renders these compounds highly advantageous for use in therapy, particularly in the treatment or prevention of pain, an autoimmune disorder, an autoinflammatory disorder, an inflammatory disorder, a central nervous system disorder, spinal cord injury, a metabolic disorder, a gastrointestinal disorder, a cardiovascular disorder, a fibrotic disorder, a respiratory disorder, a skin disorder, an allergic disorder, or cancer.

### The Protease-Activated Receptors (PARs) Family

G Protein-Coupled Receptors (GPCRs) form the largest family of human membrane proteins (˜800 members) and are involved in many physiological processes. Compounds targeting GPCRs also represent approximately 27% of the global market for therapeutic drugs (Hauser et al., Nat. Rev. Drug Discov., 2017, 16(12):829-842). 2% of the human genome code for proteases (also called proteinases) which suggests their importance in the correct functioning of the body (Hollenberg et al., Br. J. Pharmacol., 2014, 171(5):1180-94). Indeed, it has been shown that certain soluble and membrane-bound proteinases can regulate cell function by cleaving GPCRs at the cell surface to activate or inactivate receptors such as the Protease-Activated Receptors (PARs). The PARs family is composed of four members (PAR-1, PAR-2, PAR-3 and PAR-4) and belongs to the class A GPCR-receptor sub-family (Marcfarlane et al., Pharmacological Reviews, 2001, 475(7357):519-23). They are expressed in widely diverse cells such as platelets, immune cells, endothelial cells, myocytes, astrocytes, neurons, epithelial cells and fibroblasts and involved in a large set of physiological and pathophysiological functions (Ossovskaya et al., Physiol. Rev., 2004, 84(2):579-621).

### Par-2: Mechanism of Action

Activation of PARs involves the cleavage of the extracellular N-terminal part of the receptor by proteases at a specific site. This unmasks an amino-acid sequence in the amino terminus that folds back to act as a “tethered ligand” (TL): it binds to a conserved region in the second extracellular loop of the cleaved receptor and triggers intra-cellular signalling (Ossovskaya et al., Physiol. Rev., 2004, 84(2):579-621; Hollenberg et al., Br. J. Pharmacol., 2014, 171(5):1180-94).

PAR-2 is activated by several host and pathogen-derived serine proteases such as trypsin, mast cell tryptase, kallikreins and members of the coagulation cascade TF-FVIIa and FVa-FXa. These proteases cleave at R³¹↓S³⁵LIGKV and unmask the tethered ligand SLIGKV in humans. Artificially, in vitro, synthetic peptides corresponding to the TL (SLIGKV) can activate the receptor without cleavage.

Activation of PAR-2 induces several signalling cascades involving a number of G proteins such as G_{q}, Gᵢ, and G_{12/13}. The pathway best described so far involves its interaction with G_{q} and the mobilization of intracellular calcium that influences the function of several cell types. After repeated activations, PAR2 is rapidly desensitized via its endocytosis by a β-arrestin-dependent mechanism and its targeting to the lysosomes (Ossovskaya et al., Physiol. Rev., 2004, 84(2):579-621).

### Par-2 in Physiological Conditions

PAR-2 has been shown to have a key function in multiple organs (Ossovskaya et al., Physiol. Rev., 2004, 84(2):579-621). PAR-2 is expressed in the brain within neurons and glial cells. It is also found in the periphery in spinal afferent neurons and nociceptive DRG neurons. PAR-2 signalling has been involved in the survival, sensitization of these cells and their signal transmission, thereby controlling neuronal damage, inflammation and pain.

PAR-2 is involved in the function of the cardiovascular system. Indeed, its activation can induce the relaxation or contraction of some vessels such as pulmonary arteries, coronary and intramyocardial arteries, therefore regulating the blood flow. It also controls inflammation and repair of the endothelium which influences vascular permeability.

PAR-2 expression has been detected within the gastrointestinal system in the small intestine, colon, liver, pancreas and stomach. Its activation has been involved in the regulation of ion transport from the intestinal mucosa, contraction of gastric longitudinal muscle, pancreatic, salivary and gastric secretions, excitation of myenteric neurons, intestinal barrier integrity, release of prostaglandins from enterocytes. PAR-2 therefore plays a key role in controlling fluid secretion, intestinal inflammation, and gastro-intestinal hyperalgesia.

PAR-2 is involved in airways function since it is expressed by epithelial and endothelial cells in the lungs. Its activation has been shown to regulate bronchodilatation or bronchoconstriction (depending on the experimental system used), ion transport in the airway epithelium, proliferation and activation of airway smooth muscle cells and lung fibroblasts. PAR-2 can thus regulate airway resistance, lung inflammation and lung fibrosis.

In the skin, PAR-2 expression has been detected in keratinocytes, microvasculature and immune cells. Its activation has been involved in skin pigmentation, skin inflammation, and wound healing.

Finally, PAR-2 expression has been detected in immune cells such as macrophages where it influences cell maturation and cytokine secretion, thereby regulating inflammation.

### Par-2 in Pathological Conditions

Since PAR-2 regulates numerous and diverse biological processes, it is not surprising that its dysfunction is involved in as many pathological conditions.

PAR-2 is expressed in the brain, dorsal root ganglia, spinal afferent neurons and nociceptive DRG neurons. Its activation by proteases such as the tryptase released by mast cells leads to calcium and cAMP signalling (Steinhoff et al., Nat Med, 2000, 6(2):151-8; Zhao et al., J Biol Chem., 2015, 290(22):13875-87). This promotes inflammation and hyperalgesia through the release of CGRP (calcitonine gene-related peptide) and SP (substance P) from spinal afferent neurons and the sensitization of Transient Receptors Potential Vanilloid (TRPV) TRPV1 and TRPV4 in sensory neurons (Vergnolle et al., Nat Med, 2001, 6(2):151-8; Steinhoff et al., Nat Med, 2000, 6(2):151-8; Amadesi et al., J Neurosci, 2004, 24(18):4300-12; Grant et al., J Physiol, 2007, 578 (Pt 3), 715-33; Jimenez Vargas et al., Proc Natl Acad Sci USA, 2018, 115(31):E7438-E7447). This is supported by the large amount ofin vivo data available in the literature demonstrating that inhibition of PAR-2 reduces inflammatory pain, neuropathic pain, cancer pain and treatment-induced pain in animal models (Bao et al., Expert Opin Ther Targets, 2014; 18(1):15-27; Chen et al., Neuroscience, 2011, 193, 440-51). PAR-2 is therefore clearly involved in the generation and the transmission of the pain signal, neurogenic inflammation and nociception.

The expression of PAR-2 and proteases is elevated in the spinal cord after a contusion-compression injury (Radulovic et al., Neurobiol Dis, 2015, 83, 75-89; Li et al, Physiol. Res., 2019, 68(2):305-316). Its activation can result in cAMP signalling in oligodendrocytes (Yoon et al., Glia, 2017, 65(12):2070-2086). Experiments in vitro and in vivo in rodents have shown that the inhibition of PAR-2 signalling during experimental spinal cord injury reduces inflammation, scar formation and mechanical and thermal hyperalgesia and improves remyelination of oligodendrocytes and locomotor recovery (Radulovic et al., Neurobiol Dis, 2015, 83, 75-89; Li et al, Physiol. Res., 2019, 68(2):305-316; Yoon et al., Glia, 2017, 65(12):2070-2086; Li et al, Physiol. Res., 2019, 68(2):305-316; Wei et al, Physiol. Res., 2016, 65(1):145-53). PAR-2 inhibitors can thus improve recovery from spinal cord injuries.

Disorders of the immune system are at the basis of numerous diseases. In all cases, the immune system attacks the normal constituents of the organism considering them as foreign. It becomes pathogenic and induces lesions on a specific organ (e.g., type 1 diabetes in the pancreas or multiple sclerosis in the brain) or systemically (e.g., rheumatoid arthritis or systemic lupus erythematosus, SLE).

Cytokines are small proteins involved in cell signalling that orchestrate the immune response. Their dysregulation is at the basis of the pathogenesis of autoinflammatory diseases. These conditions are characterized by immune activation, infiltration and abnormal cytokine production. They include conditions such as: rheumatologic inflammatory diseases, skin inflammatory diseases, lung inflammatory diseases, muscle inflammatory diseases, bowel inflammatory diseases, brain inflammatory diseases and autoimmune diseases.

While autoinflammatory diseases evolve chronically, some conditions can lead to an acute immune disorder. Indeed, a sudden excessive and uncontrolled release of pro-inflammatory cytokines, also called cytokine storm, has been observed in graft-versus-host disease, multiple sclerosis, pancreatitis, multiple organ dysfunction syndrome, viral diseases, bacterial infections, hemophagocytic lymphohistiocytosis, and sepsis (Gerlach H, F1000Res, 2016, 5, 2909; Tisoncik J R et al., Microbiol Mol Biol Rev, 2012, 76(1):16-32). In these conditions, a dysregulated immune response and subsequent hyperinflammation may lead to multiple organ failure that can be fatal.

Because PAR-2 influences the production of inflammatory cytokines and the function of diverse organs, numerous studies have demonstrated that it is a promising therapeutic target for various autoinflammatory diseases.

The expression of proteinases and PAR-2 is significantly increased in organs directly involved in autoinflammatory diseases such as the coronary arteries of atherosclerotic patients (Jones et al., Arterioscler Thromb Vasc Biol, 2018, 38(6):1271-1282), the skin of atopic dermatitis and psoriasis patients (Nattkemper et al., Journal of Investigative Dermatology, 2018, 138:1311-1317), the joints of rheumatoid arthritis and osteoarthritis patients (Tindell et al., Rheum Int, 2012, 32(10):3077-86), the colon of inflammatory bowel disease patients (Christerson et al., J Crohns Colitis, 2009, 3(1):15-24; Kim et al., Inflamm Bowel Dis., 2003, 9(4):224-9), the lungs of idiopathic pulmonary fibrosis patients (Bardou et al., Am J Respir Crit Care Med, 2016, 193(8):847-60), the liver of non-alcoholic steatohepatitis patients (Rana et al., Mol Metab, 2019, 29:99-113), the area of active demyelination in the brain of multiple sclerosis patients (Noorbakhsh et al., J Exp Med, 2006, 203(2):425-35).

There, PAR-2 activation leads to calcium signalling in several cells such as osteoblasts, fibroblasts, monocytes, keratinocytes (Abraham et al, Bone, 2000, 26(1):7-14; Lin et al., J. Cell. Mol. Med., 2015, 19(6):1346-56; Johansson et al., J leukoc Biol, 2005, 78(4):967-75; Joo et al., Bio Mol Ther, 2016, 24(5):529-535). This signalling is associated with cell maturation and/or migration, activation as well as the secretion of inflammatory cytokines such as IL-8, IL-6, TNFα and IL-1β in various cell types such as vascular smooth muscle cells, synovial cells, monocytes, keratinocytes, astrocytes, chondrocytes, adipocytes and fibroblasts (Demetz et al., Atherosclerosis, 2010, 212:466-471; Kelso et al., Arthritis Rheum, 2007, 56(3):765-71; Johansson et al., J Leukoc Biol, 2005, 78(4):967-75; Steven et al., Innate Immun, 2013, 19(6):663-72; Kim et al., Bio Mol Ther, 2012, 20(5):463-9; Radulovic et al., Neurobiol Dis, 2015, 83, 75-89; Lin et al., J. Cell. Mol. Med., 2015, 19(6):1346-56; Bagher et al., Cell Communi and Signal, 2018, 16(1), 59; Huang et al, Aging, 2019, 11(24):12532-12545; Bandeanlou et al., Nat. Med., 2011, 17:1490-1497). PAR-2 signalling also influences tissue remodelling through its role in the survival of key cells such as neurons and chondrocytes in central nervous system disorders and rheumatologic inflammatory diseases respectively (Afkhami-Goli et al., J Immunol, 2007, 179(8):5493-503; Huang et al., Aging, 2019, 11(24):12532-12545), as well as the secretion of growth factors (e.g. CTGF) and extracellular components (e.g. collagen) (Lin et al., Mol. Med., 2015, 21(1):576-83; Chung et al., J Biol Chem, 2013, 288(52):37319-31). It is important to note that other signalling pathways such as cyclic AMP in alveolar macrophages and Gᵢ in hepatocytes seem important to regulate cytokine secretion and steatosis respectively (Rayees et al., Cell Rep, 2019, 27(3):793-805.e4; Rana et al., Mol Metab, 2019, 29, 99-113).

In vivo, it has clearly been shown that the inhibition of PAR-2 signaling, either pharmacologically or by genetic modification, significantly reduced the symptoms of atherosclerosis, idiopathic pulmonary fibrosis, atopic dermatitis, multiple sclerosis, arthritis, non-alcoholic steatohepatitis and inflammatory bowel disease in mouse models (Jones et al., Arterioscler Thromb Vasc Biol, 2018, 38(6):1271-1282; Borensztajn et al., Am J Pathol, 2010, 177(6):2753-64; Moniaga et al., Am J Pathol, 2013, 182: 841e851; Noorbakhsh et al., J Exp Med, 2006, 203(2):425-35, Ferrell et al., J Clin Invest, 2003, 111(1):35-41; Rana et al., Mol Metab, 2019, 29:99-113; Hyun et al., Gut, 2008, 57(9):1222-9). PAR-2 therefore plays a key role in the molecular and cellular mechanisms underlying the pathogenesis of autoinflammatory diseases.

PAR-2-dependent inflammation can also impair cellular metabolism and promote insulin resistance which then leads to the pathogenesis of diabetes, obesity and metabolic syndrome. Indeed, PAR-2 expression in adipocyte tissues has been correlated with the increasing BMI of volunteer people and the inhibition of PAR-2 signaling attenuates the symptoms of metabolic disorders in mice (Lim et al., FASEB Journal, 2013, 27(12):4757-4767; Badeanlou et al., Nat. Med., 2011, 17(11):1490-1497).

Many airborne allergens from house dust mite and cockroach allergens contain protease activity. This protease activity can activate PAR-2 expressed on human airway epithelial cells, endothelial cells as well as immune cells and induce calcium signalling. This ultimately leads to the release of inflammatory cytokines and angiogenic response at the basis of the pathogenesis of cockroach allergy and allergic asthma (Do et al., Allergy, 2016, 71(4):463-74; Asosingh et al., J Clin Invest, 2018, 128(7):3116-3128). In vivo, functional blockade of PAR-2 in the airways during allergen challenge improves allergen-induced inflammation and airway hyperresponsiveness in mice (Asaduzzaman et al., Clin Exp Allergy, 2015, 45(12):1844-55).

The expression of PAR-2 and proteases is also significantly increased in many cancer types such as cervical squamous cell carcinoma, endocervical adenocarcinoma, colon adenocarcinoma, esophageal carcinoma, glioblastoma multiforme, acute myeloid leukemia, lung adenocarcinoma, lung squamous cell carcinoma, ovarian serous cystadenocarcinoma, pancreatic adenocarcinoma, prostate adenocarcinoma, rectum adenocarcinoma, stomach adenocarcinoma, testicular germ cell tumors, uterine corpus endometrial carcinoma, uterine carcinosarcoma, hepatocellular carcinoma, and breast cancer, which can be associated to poor prognosis (Kaufmann et al., Carcinogenesis, 2009, 30(9):1487-96; Su et al., Oncogene, 2009, 28(34):3047-57; Arakaki et al., Int. J. Mol. Sci. 2018, 19, 1886). The activation of this receptor in cancer cells can lead to several signalling cascades such as calcium, 1-arrestin and Gᵢ signalling (Kaufmann et al., J Cancer Res Clin Oncol, 2011, 137(6):965-73; Wu et al, Mol Med Rep, 2014, 10(6):3021-6; Ge et al., J Biol Chem, 2004, 279(53):55419-24). This ultimately controls cancer cell migration, proliferation, survival, and expression of inflammatory cytokines (Jiang et al., J Pharmacol Exp Ther, 2018, 364(2):246-257; Darmoul et al., British J Cancer, 2001, 85(5):772-9; Quan et al., Oncol Res., 2019, 27(7):779-788). The expression of PAR-2 on other cells of the tumor microenvironment, such as immune cells, fibroblasts, endothelial cells and DRG neurons, can also control the immune response to cancer cells, fibrosis, as well as angiogenesis and cancer-induced pain (Mubbach et al., Mol cancer, 2016, 15(1):54; Uusitalo-Jarvinen et al., Arteriocler Thromb Vasc Biol, 2007, 27(6):1456-62; D'Andrea et al, Am J Pathol, 2001, 158(6):2031-41; Graf et al, Sci Immunol, 2019, 4(39):eaaw8405; Qian at al., Oncol Lett, 2018, 16(2):1513-20; Tu et al, J Neurosci, 2021, 41(1):193-210). In vivo, the inhibition of PAR-2 has been shown to be an efficient way of reducing tumor growth and increasing survival in mouse models of different cancers such as breast cancer, liver cancer and colon cancer (Versteeg et al., Cancer Res, 2008, 68(17):7219-27; Sun et al., World J Gastroenterol, 2018, 24(10):1120-1133; Quan et al., Oncol Res., 2019, 27(7):779-788). Importantly, inhibition of PAR2 or one of its ligands led to reduced infiltration of immune-supressive Tumor Associated Macrophages and regulatory T cells while increasing cytotoxic T cells in the tumor as well as increasing antigen presenting cells in the draining lymph nodes in several syngeneic mouse models; this unleashed the anti-tumoral immune response and increased the potency of immune-checkpoint inhibitors currently used in the clinic (Graf et al, Sci Immunol, 2019, 4(39):eaaw8405). PAR-2 therefore constitutes a promising therapeutic target in oncology and immune-oncology.

Considering the role of PAR-2 in several pathophysiological conditions, inhibitors of this receptor can have therapeutic applications in a wide variety of human diseases. This has drawn a great interest from pharmaceutical industry to develop such compounds. Various PAR-2 inhibitors and therapeutic uses thereof have been proposed, for example, in: Yau et al., Expert Opin Ther Pat, 2016, 26(4):471-83; Jiang et al., J Pharmacol Exp Ther, 2018, 364(2):246-57; WO 2004/002418; WO 2005/030773; WO 2012/012843; WO 2012/026765; WO 2012/026766; WO 2012/101453; WO 2015/048245; WO 2016/154075; WO 2017/194716; WO 2017/197463; WO 2018/043461 (EP 3 508 487); WO 2018/057588; WO 2019/163956 (EP 3 760 631); WO 2019/199800; JP 2020/007262; and WO 2021/106864. However, despite the efforts made in the past 10 years, no PAR-2 inhibitor has reached the market yet (Yau et al., Expert Opin Ther Pat., 2016, 26(4):471-83). There is therefore still an unmet need for novel and/or improved PAR-2 inhibitors with high potency, selectivity and bioavailability.

The present invention addresses this need and solves the problem of providing novel and highly potent PAR-2 inhibitors. In particular, it has surprisingly been found that the compounds of formula (I) as provided herein are potent inhibitors of PAR-2 signalling, which renders these compounds advantageous for use in therapy, including in particular in the treatment or prevention of pain, an autoimmune disorder, an autoinflammatory disorder, an inflammatory disorder, a central nervous system disorder, spinal cord injury, a metabolic disorder, a gastrointestinal disorder, a cardiovascular disorder, a fibrotic disorder, a respiratory disorder, a skin disorder, an allergic disorder, or cancer.

Accordingly, the present invention provides a compound of the following formula (I)

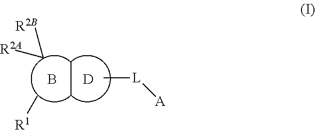

or a pharmaceutically acceptable salt or solvate thereof.

In formula (I), ring B is a non-aromatic C₄₋₈ carbocyclic ring or a non-aromatic 4- to 8-membered heterocyclic ring, which is fused to ring D, wherein said carbocyclic ring or said heterocyclic ring is: (i) substituted with a group R¹; (ii) substituted with the groups R^{2A} and R^{2B} which are attached to the same ring carbon atom of said carbocyclic ring or said heterocyclic ring; and (iii) optionally substituted with one or more groups R^{Y}.

Ring D is a 5- or 6-membered heteroaromatic ring, which is fused to ring B, wherein said heteroaromatic ring comprises at least one nitrogen ring atom, wherein said heteroaromatic ring is substituted with a group -L-A, and wherein said heteroaromatic ring is optionally substituted with one or more groups R^{X}.

R¹ is selected from C₁₋₅ alkyl, C₂₋₅ alkenyl, C₂₋₅ alkynyl, —(C₀₋₅ alkylene)-carbocyclyl, and —(C₀₋₅ alkylene)-heterocyclyl, wherein said alkyl, said alkenyl, said alkynyl, the alkylene group in said —(C₀₋₅ alkylene)-carbocyclyl, and the alkylene group in said —(C₀₋₅ alkylene)-heterocyclyl are each optionally substituted with one or more groups R¹², wherein one or more —CH₂— units comprised in said alkyl, said alkenyl, said alkynyl, in the alkylene group in said —(C₀₋₅ alkylene)-carbocyclyl, or in the alkylene group in said —(C₀₋₅ alkylene)-heterocyclyl are each optionally replaced by a group independently selected from —C(R^{L1})(R^{L1})—, —O—, —S—, —SO—, —SO₂—, —CO—, and —N(R^{L1})—, wherein each R^{L1} is independently hydrogen or C₁₋₅ alkyl, wherein two groups R^{L1} which are attached to the same carbon atom may also be mutually joined to form, together with the carbon atom that they are attached to, a cycloalkyl or a heterocycloalkyl, and wherein the carbocyclyl group in said —(C₀₋₅ alkylene)-carbocyclyl and the heterocyclyl group in said —(C₀₋₅ alkylene)-heterocyclyl are each optionally substituted with one or more groups R¹¹.

Each R¹¹ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}.

Each R¹² is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), carbocyclyl, heterocyclyl, and -L^{Z}-R^{Z}, wherein said carbocyclyl and said heterocyclyl are each optionally substituted with one or more groups R^{Cyc}.

R^{2A} and R^{2B} are mutually joined to form, together with the carbon atom that they are attached to, a cycloalkyl, cycloalkenyl, heterocycloalkyl, or heterocycloalkenyl, wherein said cycloalkyl, said cycloalkenyl, said heterocycloalkyl or said heterocycloalkenyl is optionally substituted with one or more groups R²¹;
or R^{2A} and R^{2B} are each independently selected from C₁₋₅ alkyl, C₂₋₅ alkenyl, C₂₋₅ alkynyl, —(C₀₋₅ alkylene)-carbocyclyl, and —(C₀₋₅ alkylene)-heterocyclyl, wherein said alkyl, said alkenyl, said alkynyl, the alkylene group in said —(C₀₋₅ alkylene)-carbocyclyl, and the alkylene group in said —(C₀₋₅ alkylene)-heterocyclyl are each optionally substituted with one or more groups R²², wherein one or more —CH₂— units comprised in said alkyl, said alkenyl, said alkynyl, in the alkylene group in said —(C₀₋₅ alkylene)-carbocyclyl, or in the alkylene group in said —(C₀₋₅ alkylene)-heterocyclyl are each optionally replaced by a group independently selected from —O—, —NH—, —N(C₁₋₅ alkyl)-, —CO—, —S—, —SO—, and —SO₂—, and further wherein the carbocyclyl group in said —(C₀₋₅ alkylene)-carbocyclyl and the heterocyclyl group in said —(C₀₋₅ alkylene)-heterocyclyl are each optionally substituted with one or more groups R^{Cyc},
wherein R^{2A} and a group R^{Y} may also be mutually joined to form, together with the ring atoms that they are attached to, a carbocyclyl or heterocyclyl, wherein said carbocyclyl or said heterocyclyl is optionally substituted with one or more groups R^{Cyc}.

Each R²¹ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}.

Each R²² is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), carbocyclyl, heterocyclyl, and -L^{Z}-R^{Z}, wherein said carbocyclyl and said heterocyclyl are each optionally substituted with one or more groups R^{Cyc}.

Each R^{X} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}.

Each R^{Y} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc};
wherein any two groups R^{Y} which are attached to the same ring carbon atom (i) may also be mutually joined to form, together with the carbon atom that they are attached to, a cycloalkyl or a heterocycloalkyl, wherein said cycloalkyl or said heterocycloalkyl is optionally substituted with one or more groups R^{Cyc}, or (ii) may mutually form a group ═O.

L is selected from —CO—, —SO— and —SO₂—.

The group A is —N(—R^{N})—R^{N} or heterocyclyl, wherein said heterocyclyl is attached via a ring nitrogen atom to group L, and wherein said heterocyclyl is optionally substituted with one or more groups R^{A}.

Each R^{N} is independently selected from hydrogen, C₁₋₈ alkyl, C₂₋₈ alkenyl, C₂₋₈ alkynyl, —(C₀₋₈ alkylene)-OH, —(C₀₋₈ alkylene)-O(C₁₋₅ alkyl), —(C₀₋₈ alkylene)-SH, —(C₀₋₈ alkylene)-S(C₁₋₅ alkyl), —(C₁₋₈ alkylene)-NH₂, —(C₁₋₈ alkylene)-NH(C₁₋₅ alkyl), —(C₁₋₈ alkylene)-N(C₁₋₅ alkyl)(C₁₋₅ alkyl), —(C₁₋₈ alkylene)-halogen, —(C₁₋₈ alkylene)-C₁₋₅ haloalkyl, —(C₀₋₈ alkylene)-O—(C₁₋₈ haloalkyl), —(C₀₋₈ alkylene)-CN, —(C₀₋₈ alkylene)-CHO, —(C₀₋₈ alkylene)-CO—(C₁₋₅ alkyl), —(C₀₋₈ alkylene)-COOH, —(C₀₋₈ alkylene)-CO—O—(C₁₋₅ alkyl), —(C₀₋₈ alkylene)-O—CO—(C₁₋₅ alkyl), —(C₀₋₈ alkylene)-CO—NH₂, —(C₀₋₈ alkylene)-CO—NH(C₁₋₅ alkyl), —(C₀₋₈ alkylene)-CO—N(C₁₋₅ alkyl)(C₁₋₅ alkyl), —(C₁₋₈ alkylene)-NH—CO—(C₁₋₅ alkyl), —(C₁₋₈ alkylene)-N(C₁₋₅ alkyl)-CO—(C₁₋₅ alkyl), —(C₁₋₈ alkylene)-NH—COO(C₁₋₅ alkyl), —(C₁₋₈ alkylene)-N(C₁₋₅ alkyl)-COO(C₁₋₅ alkyl), —(C₀₋₈ alkylene)-O—CO—NH(C₁₋₅ alkyl), —(C₀₋₈ alkylene)-O—CO—N(C₁₋₅ alkyl)(C₁₋₅ alkyl), —(C₀₋₈ alkylene)-SO₂—NH₂, —(C₀₋₈ alkylene)-SO₂—NH(C₁₋₅ alkyl), —(C₀₋₈ alkylene)-SO₂—N(C₁₋₅ alkyl)(C₁₋₅ alkyl), —(C₁₋₈ alkylene)-NH—SO₂—(C₁₋₅ alkyl), —(C₁₋₈ alkylene)-N(C₁₋₅ alkyl)-SO₂—(C₁₋₅ alkyl), —(C₀₋₈ alkylene)-SO—(C₁₋₅ alkyl), —(C₀₋₈ alkylene)-SO₂—(C₁₋₅ alkyl), —(C₀₋₈ alkylene)-carbocyclyl, and —(C₀₋₈ alkylene)-heterocyclyl, wherein one or more —CH₂— units comprised in said C₁₋₈ alkyl, said C₂₋₈ alkenyl, said C₂₋₈ alkynyl, and in any of the aforementioned C₀₋₈ alkylene and C₁₋₈ alkylene groups are each optionally replaced by a group independently selected from —O—, —NH—, —N(C₁₋₅ alkyl)-, —CO—, —S—, —SO—, and —SO₂—, wherein the carbocyclyl group in said —(C₀₋₈ alkylene)-carbocyclyl and the heterocyclyl group in said —(C₀₋₈ alkylene)-heterocyclyl are each optionally substituted with one or more groups R^{Cyc}, and wherein at least one group R^{N} is not hydrogen.

Each R^{A} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}; and further wherein any two groups R^{A}, which are attached to the same carbon atom of group A, may also be mutually joined to form, together with the carbon atom that they are attached to, a cycloalkyl or a heterocycloalkyl, wherein said cycloalkyl or said heterocycloalkyl is optionally substituted with one or more groups R^{Cyc}.

Each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), —P(═O)(—OH)(—OH), —P(═O)(—OH)(—O—C₁₋₅ alkyl), —P(═O)(—O—C₁₋₅ alkyl)(—O—C₁₋₅ alkyl), —(C₀₋₃ alkylene)-cycloalkyl, —(C₀₋₃ alkylene)-heterocycloalkyl, and -L^{Z}-R^{Z}.

Each L^{Z} is independently selected from a covalent bond, C₁₇ alkylene, C₂₇ alkenylene, and C₂₇ alkynylene, wherein said alkylene, said alkenylene and said alkynylene are each optionally substituted with one or more groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—.

Each R^{Z} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, and heterocycloalkyl, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), carbocyclyl, and heterocyclyl, wherein said carbocyclyl and said heterocyclyl are each optionally substituted with one or more groups independently selected from C₁₋₅ alkyl, C₂₋₅ alkenyl, C₂₋₅ alkynyl, halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), and —SO₂—(C₁₋₅ alkyl).

Preferably, the following proviso applies to the compounds of formula (I):
if ring B is a pyrrolidinyl ring, ring D is a pyridinyl ring, R^{2A} and R^{2B} are mutually joined to form, together with the carbon atom that they are attached to, a cyclopropyl, L is —CO—, and group A is morpholin-4-yl, then R¹ is not 5-R¹¹-pyrimidin-2-yl or acetyl;
if ring B and ring D together are a 2-oxo-2,3-dihydro-1H-pyrrolo[2,3-b]pyridinyl ring, a 2-oxo-2,3-dihydro-1H-pyrrolo[2,3-c]pyridinyl ring or a 6-oxo-6,7-dihydro-5H-pyrrolo[2,3-c]pyridazinyl ring, R^{2A} and R^{2B} are each methyl, R¹ is phenyl which is optionally substituted with one or more groups R¹¹, L is —CO—, and group A is —NH—R^{N}, then R^{N} is not a heterocycloalkyl which comprises one oxidized sulfur ring atom, in which all other ring atoms are carbon atoms, and which is substituted with a methyl group;
if ring B and ring D together are a 3-R^{X}-4-oxo-4,5,6,7-tetrahydropyrazolo[1,5-a]pyrazinyl ring, R^{X} is —OH, one of R^{2A} and R^{2B} is methyl, the other one of R^{2A} and R^{2B} is —CON(—CH₃)₂, R¹ is methyl, L is —CO—, and group A is —NH—R^{N}, then R^{N} is not 4-fluorobenzyl; and
if ring B and ring D together are a 2-oxo-2,3-dihydro-1H-pyrrolo[3,2-b]pyridine ring, a 2-oxo-2,3-dihydro-1H-pyrrolo[2,3-b]pyridine ring or a 2-oxo-2,3-dihydro-1H-pyrrolo[3,2-c]pyridine, L is —CO—, group A is —NH—R^{N}, and R¹ is —CH₂-phenyl or —CH₂-pyridinyl, wherein the phenyl in said —CH₂-phenyl and the pyridinyl in said —CH₂-pyridinyl are each optionally substituted with one or more groups R¹¹, then R^{2A} and R^{2B} are not methyl.

The present invention also relates to a pharmaceutical composition comprising a compound of formula (I), or a pharmaceutically acceptable salt or solvate thereof, in combination with a pharmaceutically acceptable excipient. Accordingly, the invention relates to a compound of formula (I) or a pharmaceutically acceptable salt or solvate thereof, or a pharmaceutical composition comprising any of the aforementioned entities and a pharmaceutically acceptable excipient, for use as a medicament.

The invention further relates to a compound of formula (I) or a pharmaceutically acceptable salt or solvate thereof, or a pharmaceutical composition comprising any of the aforementioned entities and a pharmaceutically acceptable excipient, for use in the treatment or prevention of a PAR-2 mediated disease or disorder. Thus, the invention in particular provides a pharmaceutical composition comprising, as an active ingredient, a compound of formula (I) or a pharmaceutically acceptable salt or solvate thereof, together with a pharmaceutically acceptable excipient, for use in the treatment or prevention of a PAR-2 mediated disease or disorder.

Moreover, the present invention relates to the use of a compound of formula (I) or a pharmaceutically acceptable salt or solvate thereof in the preparation of a medicament for the treatment or prevention of a PAR-2 mediated disease or disorder.

The invention likewise relates to a method of treating or preventing a PAR-2 mediated disease or disorder, the method comprising administering a compound of formula (I) or a pharmaceutically acceptable salt or solvate thereof, or a pharmaceutical composition comprising any of the aforementioned entities in combination with a pharmaceutically acceptable excipient, to a subject (preferably a human) in need thereof. It will be understood that a therapeutically effective amount of the compound of formula (I) or the pharmaceutically acceptable salt or solvate thereof (or of the pharmaceutical composition) is to be administered in accordance with this method.

As explained above, the disease or disorder to be treated or prevented with a compound of formula (I) or a pharmaceutically acceptable salt or solvate thereof (or a corresponding pharmaceutical composition) in accordance with the present invention includes any PAR-2 mediated disease or disorder. It is preferred that the disease/disorder to be treated or prevented in accordance with the invention is pain (e.g., chronic pain), an autoimmune disorder, an autoinflammatory disorder, an inflammatory disorder (e.g., a rheumatologic inflammatory disorder, a skin inflammatory disorder, a lung inflammatory disorder, a muscle inflammatory disorder, a bowel inflammatory disorder, or a brain inflammatory disorder), a central nervous system disorder, spinal cord injury, a metabolic disorder, a gastrointestinal disorder, a cardiovascular disorder, a fibrotic disorder, a respiratory disorder, a skin disorder, an allergic disorder, or cancer. More preferably, the disease/disorder to be treated or prevented in accordance with the present invention is selected from neuropathic pain, inflammatory pain, cancer pain, post-operative incision pain, fracture pain, osteoporotic fracture pain, gout joint pain, chronic pain, spinal cord injury, atopic dermatitis, contact dermatitis, dry skin dermatitis, seborrhoeic dermatitis, arthritis, rheumatoid arthritis, osteoarthritis, psoriasis, psoriatic arthritis, multiple sclerosis, non-alcoholic steatohepatitis (NASH), obesity (e.g., diet-induced obesity), diabetes (e.g., type 1 diabetes or type 2 diabetes), adipose inflammation, pancreatitis, metabolic syndrome, PAR-2 associated metabolic dysfunction, periodontitis, gingivitis, inflammatory bowel disease, Crohn's disease, ulcerative colitis, peptic ulcer disease (e.g., gastric ulcer or duodenal ulcer), infectious enteritis, irritable bowel syndrome, atherosclerosis, asthma, interstitial lung disease, pulmonary fibrosis (e.g., idiopathic pulmonary fibrosis), rheumatoid arthritis-associated interstitial lung disease, liver fibrosis, cystic fibrosis, renal fibrosis, peritoneal fibrosis, pancreatic fibrosis, intestinal fibrosis, cardiac fibrosis, skin fibrosis, systemic lupus erythematosus (SLE), scleroderma, skin eczema, acne, rosacea, post-inflammatory hyperpigmentation, lichen planus, pruritus, polymyositis, vasculitis, Wegener's granulomatosis (or granulomatosis with polyangiitis), Netherton syndrome, dermatomyositis, uveitis, liver cirrhosis, Alzheimer's disease, Parkinson's disease, dust mite allergy (e.g., house dust mite allergy), cockroach allergy, allergic asthma, colorectal cancer, colon cancer (e.g., colon adenocarcinoma), gastric cancer (e.g., stomach adenocarcinoma), rectal cancer (e.g., rectum adenocarcinoma), liver cancer (e.g., hepatocellular carcinoma), breast cancer, pancreatic cancer (e.g., pancreatic adenocarcinoma or pancreatic ductal adenocarcinoma), cervical cancer (e.g., cervical squamous cell carcinoma or endocervical adenocarcinoma), prostate cancer (e.g., prostate adenocarcinoma), ovarian cancer (e.g., ovarian serous cystadenocarcinoma), endometrial cancer (e.g., uterine corpus endometrial carcinoma), uterine sarcoma (e.g., uterine carcinosarcoma), germ cell cancer (e.g., testicular germ cell cancer), esophageal cancer, leukemia (e.g., acute myeloid leukemia), lung cancer (e.g., lung adenocarcinoma or lung squamous cell carcinoma), adrenal gland cancer (e.g., adrenocortical carcinoma), bile duct cancer (e.g., cholangio carcinoma), bladder cancer (e.g., bladder urothelial carcinoma), head and neck cancer, kidney cancer (e.g., kidney chromophobe, kidney renal cell carcinoma, kidney renal clear cell carcinoma, or kidney renal papillary cell carcinoma), lymphoma (e.g., lymphoid neoplasm diffuse large B-cell lymphoma), mesothelioma, sarcoma, melanoma (e.g., skin cutaneous melanoma, or uveal melanoma), thyroid carcinoma, thymus cancer (e.g., thymoma), or glioblastoma.

Accordingly, the present invention particularly relates to a compound of formula (I) or a pharmaceutically acceptable salt or solvate thereof, or a pharmaceutical composition comprising any of the aforementioned entities and a pharmaceutically acceptable excipient, for use in the treatment or prevention of neuropathic pain, inflammatory pain, cancer pain, post-operative incision pain, fracture pain, osteoporotic fracture pain, goutjoint pain, chronic pain, spinal cord injury, atopic dermatitis, contact dermatitis, dry skin dermatitis, seborrhoeic dermatitis, arthritis, rheumatoid arthritis, osteoarthritis, psoriasis, psoriatic arthritis, multiple sclerosis, non-alcoholic steatohepatitis (NASH), obesity (e.g., diet-induced obesity), diabetes, adipose inflammation, pancreatitis, metabolic syndrome, PAR-2 associated metabolic dysfunction, periodontitis, gingivitis, inflammatory bowel disease, Crohn's disease, ulcerative colitis, peptic ulcer disease (e.g., gastric ulcer or duodenal ulcer), infectious enteritis, irritable bowel syndrome, atherosclerosis, asthma, interstitial lung disease, pulmonary fibrosis (e.g., idiopathic pulmonary fibrosis), rheumatoid arthritis-associated interstitial lung disease, liver fibrosis, cystic fibrosis, renal fibrosis, peritoneal fibrosis, pancreatic fibrosis, intestinal fibrosis, cardiac fibrosis, skin fibrosis, systemic lupus erythematosus (SLE), scleroderma, skin eczema, acne, rosacea, post-inflammatory hyperpigmentation, lichen planus, pruritus, polymyositis, vasculitis, Wegener's granulomatosis (or granulomatosis with polyangiitis), Netherton syndrome, dermatomyositis, uveitis, liver cirrhosis, Alzheimer's disease, Parkinson's disease, dust mite allergy (e.g., house dust mite allergy), cockroach allergy, allergic asthma, or cancer (e.g., colorectal cancer, colon cancer (e.g., colon adenocarcinoma), gastric cancer (e.g., stomach adenocarcinoma), rectal cancer (e.g., rectum adenocarcinoma), liver cancer (e.g., hepatocellular carcinoma), breast cancer, pancreatic cancer (e.g., pancreatic adenocarcinoma or pancreatic ductal adenocarcinoma), cervical cancer (e.g., cervical squamous cell carcinoma or endocervical adenocarcinoma), prostate cancer (e.g., prostate adenocarcinoma), ovarian cancer (e.g., ovarian serous cystadenocarcinoma), endometrial cancer (e.g., uterine corpus endometrial carcinoma), uterine sarcoma (e.g., uterine carcinosarcoma), germ cell cancer (e.g., testicular germ cell cancer), esophageal cancer, leukemia (e.g., acute myeloid leukemia), lung cancer (e.g., lung adenocarcinoma or lung squamous cell carcinoma), adrenal gland cancer (e.g., adrenocortical carcinoma), bile duct cancer (e.g., cholangio carcinoma), bladder cancer (e.g., bladder urothelial carcinoma), head and neck cancer, kidney cancer (e.g., kidney chromophobe, kidney renal cell carcinoma, kidney renal clear cell carcinoma, or kidney renal papillary cell carcinoma), lymphoma (e.g., lymphoid neoplasm diffuse large B-cell lymphoma), mesothelioma, sarcoma, melanoma (e.g., skin cutaneous melanoma, or uveal melanoma), thyroid carcinoma, thymus cancer (e.g., thymoma), or glioblastoma.

The present invention also relates to a compound of formula (I) or a pharmaceutically acceptable salt or solvate thereof, wherein said compound is conjugated via a linker to a membrane anchor. The corresponding conjugate can be employed in place of the compound of formula (I) for any use or purpose described in the present specification, e.g., for use in the treatment or prevention of a PAR-2 mediated disease or disorder, including any of the diseases/disorders mentioned herein above. Such conjugates are advantageous in that they allow to tether the conjugated compound of formula (I) to a cell membrane in the proximity of PAR-2 and, thus, to facilitate its interaction with PAR-2.

The membrane anchor may be any moiety that is capable of inserting/partitioning into a lipid membrane (preferably a cell membrane), particularly a hydrophobic moiety or a lipid moiety; the conjugated compound of formula (I) is thereby “anchored” to the corresponding lipid membrane. For example, the membrane anchor may be a C₁₂₂₀ alkanoyl group (e.g., a hexadecanoyl group, —CO—(CH₂)₁₄—CH₃), cholesterol, cholestanol, a sphingolipid, or glycophosphatidylinositol (GPI). The membrane anchor may also be, e.g., a moiety of formula (II), (III), (VI), (VII), (VIII), (IX), (X), (XI), (XII), (XIII), (XIV) or (XV) as described and defined in WO 2017/197463, particularly on pages 10 to 15 of WO 2017/197463 which is incorporated herein by reference. The membrane anchor may further be, e.g., a raftophile A or A′, or a moiety of any one of the formulae 2, 200a to 200m, 3, 300a to 300g, 4a, 400aa to 400ap, 4b, 400ba, 5a, 500aa to 500ae, 5b, 500ba, 6, 600, 7, 700, 700a to 700c, 8a, 800a, 8b, 9, 900, 10, 1000, 11, 1100a, 1100b, 12, 1200a, 1200b, 13a, 1300aa to 1300ac, 13b, 1300b, 14a, 1400aa to 1400ae, 14b, 1400b, 14c, 15, 1500a, 16, 1600a, 18a, 1800a to 1800d, 18b, 19a, 1900a, 19b or 1900b, as described and defined in WO 2005/097199 which is incorporated herein by reference.

The linker is covalently bound to the membrane anchor and to the compound of formula (I) (or the pharmaceutically acceptable salt or solvate thereof). While the linker is not particularly limited, it preferably has a length of about 1 nm to about 50 nm, and/or it preferably provides a distance of at least 8 atoms between the compound of formula (I) and the membrane anchor. For example, the linker may comprise (or consist of) one or more polyethylene glycol (PEG) units, or may comprise (or consist of) a peptide (which may be composed of, e.g., 2 to 200 amino acid residues). The linker may also be, e.g., a moiety of formula (IV), (XX), (XXI) or (XXII) as described and defined in WO 2017/197463, particularly on pages 15 to 18 of WO 2017/197463 which is incorporated herein by reference. The linker may further be, e.g., a linker B or B′, or a moiety of any one of the formulae 20, 2000, 2001, 21, 2100, 2101, 22, 23, 28 or 28a, as described and defined in WO 2005/097199 which is incorporated herein by reference. It will be understood that the linker may be attached to the membrane anchor via any suitable chemical linkage, e.g. via an amide linkage or via an ester linkage. Likewise, the linker may be attached to the compound of formula (I) (or the pharmaceutically acceptable salt or solvate thereof) via any suitable chemical linkage, e.g. via an amide linkage or via an ester linkage. While the linker may be attached at any position (or to any functional group) of the compound of formula (I) or the pharmaceutically acceptable salt or solvate thereof, it is preferred that the linker is attached to group A or to a substituent R^{A} on group A.

Moreover, the linker and the membrane anchor may together form, e.g., any one of the moieties described to be attached to a PAR-2 inhibitor in WO 2017/197463 (which is incorporated herein by reference), or to a PAR-2 modulating compound in WO 2017/173347 (which is incorporated herein by reference), or to a pharmacophore in WO 2005/097199 (which is incorporated herein by reference). Suitable protocols for the preparation of corresponding linkers and membrane anchors are also described in these documents.

An example of a corresponding conjugate, wherein a compound of formula (I) is conjugated via a linker to a membrane anchor, is described in Example 261.

Accordingly, the invention provides a compound of formula (I) or a pharmaceutically acceptable salt or solvate thereof, wherein said compound is conjugated via a linker to a membrane anchor, wherein the membrane anchor is a C₁₂₂₀ alkanoyl group (e.g., a hexadecanoyl group, —CO—(CH₂)₁₄—CH₃). The invention particularly provides the compound N-(37-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-3-methyl-4,17,30,37-tetraoxo-7,10,13,20,23,26-hexaoxa-3,16,29-triazaheptatriacontyl)-N-methylpalmitamide or a pharmaceutically acceptable salt or solvate thereof.

The present invention furthermore relates to the use of a compound of formula (I) or a pharmaceutically acceptable salt or solvate thereof as an inhibitor of protease-activated receptor 2 (PAR-2) in research, particularly as a research tool compound for inhibiting PAR-2. Accordingly, the invention refers to the in vitro use of a compound of formula (I) or a pharmaceutically acceptable salt or solvate thereof as a PAR-2 inhibitor and, in particular, to the in vitro use of a compound of formula (I) or a pharmaceutically acceptable salt or solvate thereof as a research tool compound acting as a PAR-2 inhibitor. The invention likewise relates to a method, particularly an in vitro method, of inhibiting PAR-2, the method comprising the application of a compound of formula (I) or a pharmaceutically acceptable salt or solvate thereof. The invention further relates to a method of inhibiting PAR-2, the method comprising applying a compound of formula (I) or a pharmaceutically acceptable salt or solvate thereof to a test sample (e.g., a biological sample) or a test animal (i.e., a non-human test animal). The invention also refers to a method, particularly an in vitro method, of inhibiting PAR-2 in a sample (e.g., a biological sample), the method comprising applying a compound of formula (I) or a pharmaceutically acceptable salt or solvate thereof to said sample. The present invention further provides a method of inhibiting PAR-2, the method comprising contacting a test sample (e.g., a biological sample) or a test animal (i.e., a non-human test animal) with a compound of formula (I) or a pharmaceutically acceptable salt or solvate thereof. The terms “sample”, “test sample” and “biological sample” include, without being limited thereto: a cell, a cell culture or a cellular or subcellular extract; biopsied material obtained from an animal (e.g., a human), or an extract thereof; or blood, serum, plasma, saliva, urine, feces, or any other body fluid, or an extract thereof. It is to be understood that the term “in vitro” is used in this specific context in the sense of “outside a living human or animal body”, which includes, in particular, experiments performed with cells, cellular or subcellular extracts, and/or biological molecules in an artificial environment such as an aqueous solution or a culture medium which may be provided, e.g., in a flask, a test tube, a Petri dish, a microtiter plate, etc.

The compounds of formula (I) as well as the pharmaceutically acceptable salts and solvates thereof will be described in more detail in the following.

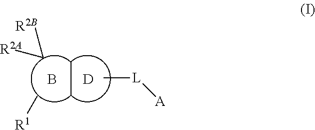

In formula (I), ring B is a non-aromatic C₄₋₈ carbocyclic ring or a non-aromatic 4- to 8-membered heterocyclic ring, which is fused to ring D, wherein said carbocyclic ring or said heterocyclic ring is: (i) substituted with a group R¹; (ii) substituted with the groups R^{2A} and R^{2B} which are attached to the same ring carbon atom of said carbocyclic ring or said heterocyclic ring; and (iii) optionally substituted with one or more (e.g., one, two or three) groups R^{Y}.

Ring D is a 5- or 6-membered heteroaromatic ring, which is fused to ring B, wherein said heteroaromatic ring comprises at least one nitrogen ring atom, wherein said heteroaromatic ring is substituted with a group -L-A, and wherein said heteroaromatic ring is optionally substituted with one or more (e.g., one, two or three) groups R^{X}.

Accordingly, as also depicted in formula (I), the rings B and D are fused, i.e., they share two adjacent ring atoms (which form part of both ring B and ring D) and thus form a fused bicyclic ring system. In this fused bicyclic ring system, only ring D is aromatic whereas ring B is non-aromatic.

As explained above, ring B is a non-aromatic C₄₋₈ carbocyclic ring or a non-aromatic 4- to 8-membered heterocyclic ring, wherein said carbocyclic ring or said heterocyclic ring is optionally substituted with one or more groups R^{Y}. It will be understood that ring B is fused to ring D, is substituted with a group R¹, and is substituted with the groups R^{2A} and R^{2B} (which are attached to the same ring carbon atom of ring B); these features are also depicted in formula (I) and will not be repeated at every instance where ring B is further described herein below.

It is preferred that said non-aromatic C₄₋₈ carbocyclic ring is a C₄₋₈ cycloalkyl ring or a C₄₋₈ cycloalkenyl ring, more preferably a C₅₇ cycloalkyl ring or a C₅₇ cycloalkenyl ring (e.g., a cyclohexenyl ring), even more preferably a cyclopentyl ring, a cyclohexyl ring, or a cycloheptyl ring.

Moreover, it is preferred that said non-aromatic 4- to 8-membered heterocyclic ring is a 4- to 8-membered heterocycloalkyl ring or a 4- to 8-membered heterocycloalkenyl ring, more preferably a 5- to 7-membered heterocycloalkyl ring (i.e., a heterocycloalkyl ring having 5, 6 or 7 ring atoms) or a 5- to 7-membered heterocycloalkenyl ring (i.e., a heterocycloalkenyl ring having 5, 6 or 7 ring atoms; e.g., a 3,4-dihydro-2H-pyranyl ring or a 3,6-dihydro-2H-pyranyl ring), even more preferably a 5- to 7-membered heterocycloalkyl ring (e.g., a pyrrolidinyl ring, a piperidinyl ring, a 1,3-diazinanyl ring, a piperazinyl ring, an azepanyl ring, a tetrahydropyranyl ring, a morpholino ring, a 1,4-oxazepanyl ring, or a 1,4-thiazepanyl ring). It is furthermore preferred that said non-aromatic heterocyclic ring (including also said heterocycloalkyl ring or said heterocycloalkenyl ring) has one or more (e.g., one or two) ring heteroatoms selected from nitrogen, oxygen and sulfur while all remaining ring atoms are carbon atoms, wherein any nitrogen ring atom (if present) and/or any sulfur ring atom (if present) is optionally oxidized, and wherein one or more carbon ring atoms are optionally oxidized (i.e., to form an oxo group). More preferably, said heterocyclic ring (including also said heterocycloalkyl ring or said heterocycloalkenyl ring) has one or more (e.g., one or two) nitrogen ring atoms while all remaining ring atoms are carbon atoms, wherein one or more carbon ring atoms are optionally oxidized; corresponding examples include, in particular, a pyrrolidinyl ring, a piperidinyl ring, a 1,3-diazinanyl ring, a piperazinyl ring, or an azepanyl ring. A particularly preferred example of said heterocyclic ring (or said heterocycloalkyl ring) is a pyrrolidinyl ring.

Preferably, ring B is a non-aromatic 4- to 8-membered heterocyclic ring (e.g., a 4- to 8-membered heterocycloalkyl ring or a 4- to 8-membered heterocycloalkenyl ring), wherein said heterocyclic ring is optionally substituted with one or more groups R^{Y}. More preferably, ring B is a non-aromatic 5- to 7-membered heterocyclic ring (e.g., a heterocycloalkyl ring having 5, 6 or 7 ring members, or a heterocycloalkenyl ring having 5, 6 or 7 ring members) which is optionally substituted with one or more R^{Y}. Even more preferably, ring B is a 5- to 7-membered heterocycloalkyl ring which is optionally substituted with one or more R^{Y}. Yet even more preferably, ring B is a 5- to 7-membered heterocycloalkyl ring which is optionally substituted with one or more R^{Y}, wherein said heterocycloalkyl ring has one or two ring heteroatoms selected from nitrogen, oxygen and sulfur (preferably one or two nitrogen ring atoms) while all remaining ring atoms are carbon atoms, wherein one or more carbon ring atoms are optionally oxidized. Corresponding preferred examples include a pyrrolidinyl ring, a piperidinyl ring, a 1,3-diazinanyl ring, a morpholinyl ring, or an azepanyl ring, particularly a pyrrolidinyl ring or a piperidinyl ring, even more preferably a pyrrolidinyl ring. For each of the aforementioned preferred definitions of ring B, it is furthermore preferred that ring B comprises at least one nitrogen ring atom and that the group R¹ is attached to said nitrogen ring atom. Accordingly, a particularly preferred example of ring B is a pyrrolidinyl ring or a piperidinyl ring, wherein said pyrrolidinyl ring or said piperidinyl ring is optionally substituted with one or more R^{Y}, and wherein the group R¹ is attached to the nitrogen ring atom of said pyrrolidinyl ring or said piperidinyl ring. An even more preferred example of ring B is a pyrrolidinyl ring (which is optionally substituted with one or more R^{Y}), wherein the group R¹ is attached to the nitrogen ring atom of said pyrrolidinyl ring.

In accordance with the above general and preferred definitions of ring B, it is particularly preferred that ring B is a group of the following formula (B1), even more preferably a group of the following formula (B2), and still more preferably a group of the following formula (B3):

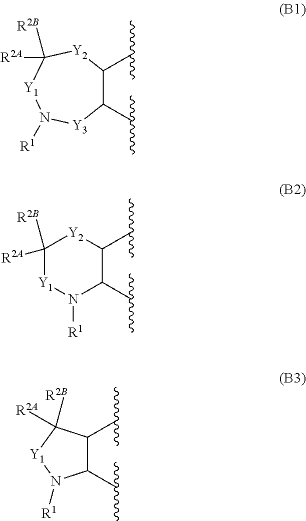

It will be understood that the above-depicted groups of formulae (B1), (B2) and (B3) are fused to ring D via the two adjacent ring carbon atoms (i.e., the two ring carbon atoms between Y₂ and Y₃ in formula (B1), the two ring carbon atoms between Y₂ and N(—R¹) in formula (B2), or the two ring carbon atoms between C(—R^{2A})(—R^{2B}) and N(—R¹) in formula (B3)), which is reflected in formulae (B1), (B2) and (B3) by the bonds (cut by a wavy line) which extend from these adjacent ring carbon atoms.

Y₁, Y₂ (if present) and Y₃ (if present) are each independently selected from a bond, —CH₂—, —CH₂—CH₂—, —O—, —S—, —SO—, —SO₂—, —CO—, —NH—, and —N(C₁₋₅ alkyl)-, wherein said —CH₂— and said —CH₂—CH₂— are each optionally substituted with one or more groups R^{Y}, wherein said —NH— is optionally substituted with a group R^{Y}, and further wherein one —CH₂-unit in said —CH₂—CH₂— is optionally replaced by a group selected from —O—, —S—, —SO—, —SO₂—, —CO—, —NH—, and —N(C₁₋₅ alkyl)-; with the proviso that formula (B1) has 4 to 8 ring atoms (i.e., the options for Y₁, Y₂ and Y₃ in formula (B1) are limited by the additional requirement that the resulting ring B is a 4- to 8-membered ring). In particular, said —CH₂— may be optionally substituted with one or two groups R^{Y}, and said —CH₂—CH₂— may be optionally substituted with one, two, three or four groups R^{Y}. It will be understood that the definitions of Y₁, Y₂ and Y₃ only apply insofar as these groups are present in the compound of formula (I), regardless of whether this is explicitly reflected by the term “if present” in the definitions of Y₁, Y₂ and Y₃. Accordingly, the definition of Y₁ applies to each of formulae (B1), (B2) and (B3), the definition of Y₂ only applies to formulae (B1) and (B2), and the definition of Y₃ only applies to formula (B1).

Preferably, Y₁, Y₂ (if present) and Y₃ (if present) are each independently selected from a bond, —CH₂—, —CH₂—CH₂—, —O—, —S—, —CO—, and —NH—, wherein said —CH₂— and said —CH₂—CH₂— are each optionally substituted with one or more (e.g., one or two) groups R^{Y}, wherein said —NH— is optionally substituted with a group R^{Y}, and wherein one —CH₂— unit in said —CH₂—CH₂— is optionally replaced by a group selected from —O—, —S—, and —CO—; with the proviso that formula (B1) has 4 to 8 ring atoms. More preferably, Y₁, Y₂ (if present) and Y₃ (if present) are each independently selected from a bond, —CH₂—, —CH₂—CH₂—, —O—, —S—, and —CO—, wherein said —CH₂— and said —CH₂—CH₂— are each optionally substituted with one or more (e.g., one or two) groups R^{Y}; with the proviso that formula (B1) has 4 to 8 ring atoms. Even more preferably, Y₁, Y₂ (if present) and Y₃ (if present) are each independently selected from a bond, —CH₂—, and —CH₂—CH₂—, wherein said —CH₂— is optionally substituted with one or two groups R^{Y}, and wherein said —CH₂—CH₂— is optionally substituted with one, two, three or four groups R^{Y}; with the proviso that formula (B1) has 4 to 8 ring atoms.

In formula (B1), it is particularly preferred that: Y₁ is selected from a bond, —CH₂—, —CH₂—CH₂—, —O—, —S—, —CO—, and —NH—, wherein said —CH₂— and said —CH₂—CH₂— are each optionally substituted with one or more (e.g., one or two) groups R^{Y}, wherein said —NH— is optionally substituted with a group R^{Y}, and wherein one —CH₂— unit in said —CH₂—CH₂— is optionally replaced by a group selected from —O—, —S—, and —CO—; and that Y₂ and Y₃ are each independently selected from a bond, —CH₂—, —O—, —S—, and —CO—, wherein said —CH₂— is optionally substituted with one or two groups R^{Y}. More preferably, Y₁ in formula (B1) is selected from a bond, —CH₂—, —CH₂—CH₂—, —O—, —S—, and —CO—, wherein said —CH₂— and said —CH₂—CH₂— are each optionally substituted with one or more (e.g., one or two) groups R^{Y}; and Y₂ and Y₃ in formula (B1) are each independently a bond or —CH₂—, wherein said —CH₂— is optionally substituted with one or two groups R^{Y}.

In formula (B2), it is preferred that Y₁ and Y₂ are each independently selected from a bond, —CH₂—, —CH₂—CH₂—, —O—, —S—, —CO—, and —NH—, wherein said —CH₂— and said —CH₂—CH₂— are each optionally substituted with one or more (e.g., one or two) groups R^{Y}, wherein said —NH— is optionally substituted with a group R^{Y}, and wherein one —CH₂— unit in said —CH₂—CH₂— is optionally replaced by a group selected from —O—, —S—, and —CO—. More preferably, Y₁ and Y₂ in formula (B2) are each independently selected from a bond, —CH₂—, —CH₂—CH₂—, —O—, —S—, and —CO—, wherein said —CH₂— and said —CH₂—CH₂— are each optionally substituted with one or more (e.g., one or two) groups R^{Y}. Even more preferably, Y₁ and Y₂ in formula (B2) are each independently selected from a bond, —CH₂—, and —CH₂—CH₂—, wherein said —CH₂— is optionally substituted with one or two groups R^{Y}, and wherein said —CH₂—CH₂— is optionally substituted with one, two, three or four groups R^{Y}.

In formula (B3), it is preferred that Y₁ is selected from a bond, —CH₂—, —CH₂—CH₂—, —O—, —S—, —CO—, and —NH—, wherein said —CH₂— and said —CH₂—CH₂— are each optionally substituted with one or more (e.g., one or two) groups R^{Y}, wherein said —NH— is optionally substituted with a group R^{Y}, and wherein one —CH₂— unit in said —CH₂—CH₂— is optionally replaced by a group selected from —O—, —S—, and —CO—. More preferably, Y₁ in formula (B3) is selected from a bond, —CH₂—, —CH₂—CH₂—, —O—, —S—, and —CO—, wherein said —CH₂— and said —CH₂—CH₂— are each optionally substituted with one or more (e.g., one or two) groups R^{Y}. Even more preferably, Y₁ in formula (B3) is selected from a bond, —CH₂—, and —CH₂—CH₂—, wherein said —CH₂— is optionally substituted with one or two groups R^{Y}, and wherein said —CH₂—CH₂— is optionally substituted with one, two, three or four groups R^{Y}. Yet even more preferably, Y₁ in formula (B3) is —CH₂— or —CH₂—CH₂—, wherein said —CH₂— is optionally substituted with one or two groups R^{Y}, and wherein said —CH₂—CH₂— is optionally substituted with one, two, three or four groups R^{Y}. Still more preferably, Y₁ in formula (B3) is —CH₂— which is optionally substituted with one or two groups R^{Y}.

Accordingly, it is particularly preferred that ring B is a group of the following formula:

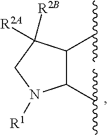

which is optionally substituted with one or two groups R^{Y}. While said formula (i.e., ring B) may be substituted with 0, 1 or 2 groups R^{Y}, it is preferably substituted with 0 or 1 group R^{Y}, and more preferably it is not substituted with any groups R^{Y}.

Thus, most preferably, ring B is a group of the following formula:

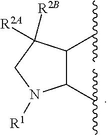

As explained above, ring D is a 5- or 6-membered heteroaromatic ring which comprises at least one nitrogen ring atom, wherein said heteroaromatic ring is optionally substituted with one or more groups R^{X}. It will be understood that ring D is fused to ring B, and that ring D is substituted with a group -L-A; these features are also depicted in formula (I) and will not be repeated at every instance where ring D is further described herein below.

Preferably, ring D is a 5- or 6-membered heteroaromatic ring having one, two or three ring heteroatoms independently selected from nitrogen, oxygen and sulfur, wherein at least one of said ring heteroatoms is a nitrogen ring atom (preferably all ring heteroatoms are nitrogen ring atoms), while all remaining ring atoms are carbon atoms, wherein said heteroaromatic ring is optionally substituted with one or more groups R^{X}. Corresponding preferred examples of ring D include a pyridinyl ring, a pyridazinyl ring, a pyrimidinyl ring, a pyrazinyl ring, an 1,2,4-triazinyl ring, a 1H-pyrrolyl ring, a pyrazolyl ring, an imidazolyl ring, a 1,2,3-triazolyl ring, a furanyl ring, a thiophenyl ring, an oxazolyl ring, an isoxazolyl ring, a thiazolyl ring, or an isothiazolyl ring. In particular, ring D may be, e.g., a 5- or 6-membered heteroaromatic ring which comprises one nitrogen ring atom and optionally one further ring heteroatom selected from nitrogen, oxygen and sulfur (preferably a further nitrogen ring atom), wherein all remaining ring atoms are carbon atoms, and wherein said heteroaromatic ring is optionally substituted with one or more groups R^{X}. More preferably, ring D is a 6-membered heteroaromatic ring which comprises one, two or three nitrogen ring atoms while all remaining ring atoms are carbon atoms, and wherein said heteroaromatic ring is optionally substituted with one or more groups R^{X}. Even more preferably, ring D is a 6-membered heteroaromatic ring which comprises one or two nitrogen ring atoms while all remaining ring atoms are carbon atoms, and wherein said heteroaromatic ring is optionally substituted with one or more groups R^{X}; corresponding examples include, in particular, a pyridinyl ring, a pyridazinyl ring, a pyrimidinyl ring, or a pyrazinyl ring. Even more preferably, ring D is a pyridinyl or pyrazinyl ring which is optionally substituted with one or more groups R^{X}. Yet even more preferably, ring D is a pyridinyl or pyrazinyl ring which is optionally substituted with one or more groups R^{X}, wherein said pyridinyl or pyrazinyl ring is fused via its ring carbon atoms 2 and 3 to ring B. Still more preferably, ring D is a pyrazinyl ring which is optionally substituted with one or more groups R^{X}, wherein said pyrazinyl ring is fused via its ring carbon atoms 2 and 3 to ring B. It is furthermore preferred that ring D is substituted with 0, 1 or 2 groups R^{X}, more preferably with 0 or 1 group R^{X}, even more preferably ring D is not substituted with any groups R^{X}.

The group -L-A is attached to ring D, as also depicted in formula (I). While the group -L-A may, in principle, be attached to any ring atom of ring D (other than the two ring atoms shared by ring B and ring D), in the case of a 5-membered heteroaromatic ring as ring D, it is preferred that -L-A is attached to the ring atom of said 5-membered heteroaromatic ring which is most distant to the two ring atoms shared by ring B and ring D. In the case of a 6-membered heteroaromatic ring as ring D, it is preferred that -L-A is attached to one of the two ring atoms of said 6-membered heteroaromatic ring which are most distant to the two ring atoms shared by ring B and ring D, more preferably to that ring atom (among the aforementioned two ring atoms which are most distant to the two ring atoms shared by ring B and ring D) which is closer (i.e., more proximate) to the ring atom of ring B carrying the groups R^{2A} and R^{2B}.

In accordance with the above general and preferred definitions of ring D, it is particularly preferred that ring D is a group of the following formula (D1):

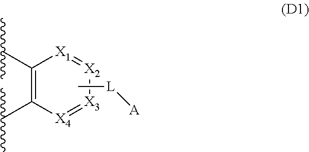

wherein the ring atoms X₁, X₂, X₃ and X₄ are each independently a carbon atom or a nitrogen atom, wherein one among X₂ and X₃ is a carbon atom and carries the group -L-A, wherein at least one of the remaining ring atoms among X₁, X₂, X₃ and X₄ is a nitrogen atom, and wherein any among the ring atoms X₁, X₂, X₃ and X₄ that is a carbon atom (and that does not carry the group -L-A) is optionally substituted with a group R^{X}.

It will be understood that the above-depicted group of the formula (D1) is fused via the two adjacent ring carbon atoms (other than X₁ to X₄) to ring B, which is reflected in formula (D1) by the bonds that extend from these adjacent ring carbon atoms (and that are cut by a wavy line). It will further be understood that if any of X₁, X₂, X₃ and X₄ is a carbon atom which is substituted with a group R^{X}, the group R^{X} replaces a hydrogen atom that would otherwise (i.e., in the absence of the optional substituent R^{X}) be attached to the respective carbon atom. Moreover, it will be understood that any among the ring atoms X₁, X₂, X₃ and X₄ that is a nitrogen atom does not carry any optional substituent.

In formula (D1), it is preferred that one or two of the ring atoms X₁, X₂, X₃ and X₄ is/are each a nitrogen atom, and all remaining ring atoms (among X₁, X₂, X₃ and X₄) are carbon atoms, whereby one of X₂ and X₃ is a carbon atom that carries the group -L-A. For example, X₁ may be a nitrogen atom while all remaining ring atoms X₂, X₃ and X₄ are carbon atoms (whereby one of X₂ and X₃ is a carbon atom that carries the group -L-A); or X₄ may be a nitrogen atom while all remaining ring atoms X₁, X₂ and X₃ are carbon atoms (whereby one of X₂ and X₃ is a carbon atom that carries the group -L-A); or X₁ and X₄ may be nitrogen atoms while the remaining ring atoms X₂ and X₃ are carbon atoms (whereby one of X₂ and X₃ is a carbon atom that carries the group -L-A). More preferably, X₁ is a nitrogen atom, one of X₂, X₃ and X₄ is a nitrogen atom or a carbon atom, and the other two of X₂, X₃ and X₄ are each a carbon atom, whereby one of X₂ and X₃ is a carbon atom that carries the group -L-A. Even more preferably, X₁ is a nitrogen atom, X₄ is a nitrogen atom or a carbon atom, and X₂ and X₃ are each a carbon atom, whereby one of X₂ and X₃ is a carbon atom that carries the group -L-A. Yet even more preferably, X₁ and X₄ are each a nitrogen atom and X₂ and X₃ are each a carbon atom, whereby the carbon atom in either X₂ or X₃ carries the group -L-A. As explained above, it is preferred that the compound of formula (I) comprises zero, one or two groups R^{X}, more preferably zero or one group R^{X}, even more preferably zero (i.e., no) groups R^{X}. Accordingly, it is particularly preferred that X₁ and X₄ are each a nitrogen atom, one of X₂ and X₃ is C(-L-A), and the other one of X₂ and X₃ is C(—H).

Still more preferably, ring D is a group of the following formula (D2):

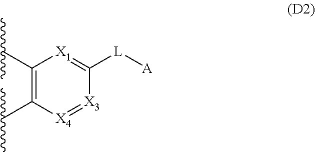

wherein the ring atoms X₁, X₃ and X₄ are each independently a carbon atom or a nitrogen atom, wherein at least one among X₁, X₃ and X₄ is a nitrogen atom, and wherein any among the ring atoms X₁, X₃ and X₄ that is a carbon atom is optionally substituted with a group R^{X}.

Preferably, one or two of the ring atoms X₁, X₃ and X₄ is/are each a nitrogen atom, and all remaining ring atoms (among X₁, X₃ and X₄) are carbon atoms. More preferably, X₁ is a nitrogen atom, one of X₃ and X₄ is a nitrogen atom or a carbon atom, and the other one of X₃ and X₄ is a carbon atom. Thus, for example, X₁ may be a nitrogen atom, and X₃ and X₄ may each be a carbon atom; or, alternatively, X₁ and X₃ may each be a nitrogen atom, and X₄ may be a carbon atom; or, alternatively, X₁ and X₄ may each be a nitrogen atom, and X₃ may be a carbon atom. It is particularly preferred that X₁ and X₄ are each a nitrogen atom, and X₃ is a carbon atom. As explained above, any among the ring atoms X₁, X₃ and X₄ that is a carbon atom is optionally substituted with a group R^{X}. Yet, it is preferred that the compound of formula (I) comprises zero, one or two groups R^{X}, more preferably zero or one group R^{X}, even more preferably zero groups R^{X}, so that any among the ring atoms X₁, X₃ and X₄ that is a carbon atom is preferably not substituted with a group R^{X}, i.e. is a ring atom C(—H). Accordingly, it is particularly preferred that X₁ and X₄ are each a nitrogen atom, and X₃ is a carbon atom optionally substituted with R^{X}; even more preferably, X₁ and X₄ are each N, and X₃ is C(—H).

Thus, even more preferably, ring D is a group of the following formula (D3):

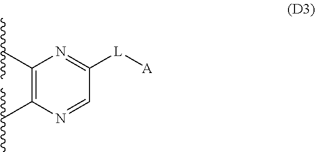

In accordance with the above definitions of rings B and D, it is particularly preferred that the compound of formula (I) has the following structure:

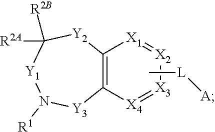

more preferably the compound of formula (I) has the following structure:

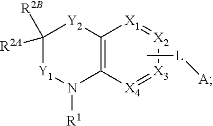

even more preferably the compound of formula (I) has the following structure:

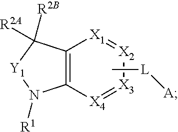

yet even more preferably the compound of formula (I) has the following structure:

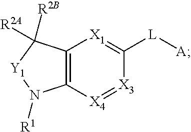

still more preferably the compound of formula (I) has the following structure:

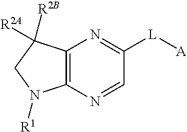

In formula (I), the group R¹ is selected from C₁₋₅ alkyl, C₂₋₅ alkenyl, C₂₋₅ alkynyl, —(C₀₋₅ alkylene)-carbocyclyl, and —(C₀₋₅ alkylene)-heterocyclyl, wherein said alkyl, said alkenyl, said alkynyl, the alkylene group in said —(C₀₋₅ alkylene)-carbocyclyl, and the alkylene group in said —(C₀₋₅ alkylene)-heterocyclyl are each optionally substituted with one or more (e.g., one, two, or three) groups R¹², wherein one or more (e.g., one, two, or three) —CH₂— units comprised in said alkyl, said alkenyl, said alkynyl, in the alkylene group in said —(C₀₋₅ alkylene)-carbocyclyl, or in the alkylene group in said —(C₀₋₅ alkylene)-heterocyclyl are each optionally replaced by a group independently selected from —C(R^{L1})(R^{L1})—, —O—, —S—, —SO—, —SO₂—, —CO—, and —N(R^{L1})—, wherein each R^{L1} is independently hydrogen or C₁₋₅ alkyl, wherein two groups R^{L1} which are attached to the same carbon atom may also be mutually joined to form, together with the carbon atom that they are attached to, a cycloalkyl or a heterocycloalkyl, and wherein the carbocyclyl group in said —(C₀₋₅ alkylene)-carbocyclyl and the heterocyclyl group in said —(C₀₋₅ alkylene)-heterocyclyl are each optionally substituted with one or more (e.g., one, two, three, or four) groups R¹¹.

Preferably, R¹ is selected from C₁₋₅ alkyl, —(C₀₋₅ alkylene)-carbocyclyl, and —(C₀₋₅ alkylene)-heterocyclyl, wherein said alkyl, the alkylene group in said —(C₀₋₅ alkylene)-carbocyclyl, and the alkylene group in said —(C₀₋₅ alkylene)-heterocyclyl are each optionally substituted with one or more groups R¹², wherein one or more —CH₂— units comprised in said alkyl, in the alkylene group in said —(C₀₋₅ alkylene)-carbocyclyl, or in the alkylene group in said —(C₀₋₅ alkylene)-heterocyclyl are each optionally replaced by a group independently selected from —C(R^{L1})(R^{L1})—, —O—, —SO—, —SO₂—, —CO—, and —N(R^{L1})—, wherein each R^{L1} is independently hydrogen or C₁₋₅ alkyl, wherein two groups R^{L1} which are attached to the same carbon atom may also be mutually joined to form, together with the carbon atom that they are attached to, a cycloalkyl (e.g., a C₃₋₆ cycloalkyl) or a heterocycloalkyl (e.g., a 3 to 6-membered heterocycloalkyl), and wherein the carbocyclyl group in said —(C₀₋₅ alkylene)-carbocyclyl and the heterocyclyl group in said —(C₀₋₅ alkylene)-heterocyclyl are each optionally substituted with one or more groups R¹¹.

More preferably, R¹ is selected from C₁₋₅ alkyl (e.g., isobutyl), —(C₀₋₅ alkylene)-carbocyclyl, and —(C₀₋₅ alkylene)-heterocyclyl, wherein the alkylene group in said —(C₀₋₅ alkylene)-carbocyclyl and the alkylene group in said —(C₀₋₅ alkylene)-heterocyclyl are each optionally substituted with one or more groups R¹², wherein one or more —CH₂— units comprised in the alkylene group in said —(C₀₋₅ alkylene)-carbocyclyl or in the alkylene group in said —(C₀₋₅ alkylene)-heterocyclyl are each optionally replaced by a group independently selected from —C(R^{L1})(R^{L1})—, —O—, —S—, —SO—, —SO₂—, —CO—, and —N(R^{L1})—, wherein each R^{L1} is independently hydrogen or C₁₋₅ alkyl, wherein two groups R^{L1} which are attached to the same carbon atom may also be mutually joined to form, together with the carbon atom that they are attached to, a cycloalkyl or a heterocycloalkyl, and wherein the carbocyclyl group in said —(C₀₋₅ alkylene)-carbocyclyl and the heterocyclyl group in said —(C₀₋₅ alkylene)-heterocyclyl are each optionally substituted with one or more groups R¹¹.

Even more preferably, R¹ is -L¹-carbocyclyl or -L¹-heterocyclyl, wherein the carbocyclyl in said -L¹-carbocyclyl or the heterocyclyl in said -L¹-heterocyclyl is optionally substituted with one or more (e.g., one, two, three, or four) groups R¹¹.

For example, R¹ may be -L¹-aryl, -L¹-cycloalkyl, -L¹-cycloalkenyl, -L¹-heteroaryl, -L¹-heterocycloalkyl, or -L¹-heterocycloalkenyl, wherein the cyclic moiety in each of the aforementioned groups is optionally substituted with one or more groups R¹¹. It is particularly preferred that R¹ is -L¹-aryl, -L¹-cycloalkyl, -L¹-heteroaryl or -L¹-heterocycloalkyl, wherein the aryl in said -L¹-aryl, the cycloalkyl in said -L¹-cycloalkyl, the heteroaryl in said -L¹-heteroaryl or the heterocycloalkyl in said -L¹-heterocycloalkyl is optionally substituted with one or more groups R¹¹. More preferably, R¹ is -L¹-aryl, -L¹-cycloalkyl or -L¹-heteroaryl, wherein the aryl in said -L¹-aryl, the cycloalkyl in said -L¹-cycloalkyl or the heteroaryl in said -L¹-heteroaryl is optionally substituted with one or more groups R¹¹. In particular, it is preferred that R¹ is selected from -L¹-phenyl, -L¹-naphthyl (e.g., -L¹-naphthalen-1-yl or -L¹-naphthalen-2-yl), -L¹-(C₃₋₇ cycloalkyl), -L¹-(monocyclic 5- or 6-membered heteroaryl), or -L¹-(bicyclic 9- or 10-membered heteroaryl), wherein the cyclic moiety in each of the aforementioned groups is optionally substituted with one or more groups R¹¹. By way of example, if R¹ is -L¹-heteroaryl [e.g., -L¹-(monocyclic 5- or 6-membered heteroaryl) or -L¹-(bicyclic 9- or 10-membered heteroaryl)] wherein the heteroaryl in said -L¹-heteroaryl is optionally substituted with one or more groups R¹¹, then the heteroaryl in said -L¹-heteroaryl may be, e.g., selected from pyrrolyl (e.g., 1H-pyrrol-1-yl, 1H-pyrrol-2-yl, or 1H-pyrrol-3-yl), pyrazolyl (e.g., pyrazol-1-yl, pyrazol-3-yl, or pyrazol-4-yl), imidazolyl (e.g., imidazol-1-yl, imidazol-2-yl, or imidazol-4-yl), triazolyl (e.g., 1H-1,2,3-triazolyl, 2H-1,2,3-triazolyl, 1H-1,2,4-triazolyl, or 4H-1,2,4-triazolyl; such as, e.g., 1H-1,2,3-triazol-1-yl, 1H-1,2,3-triazol-4-yl, 1H-1,2,3-triazol-5-yl, 1H-1,2,4-triazol-1-yl, 1H-1,2,4-triazol-3-yl, or 1H-1,2,4-triazol-5-yl), furanyl (e.g., furan-2-yl or furan-3-yl), thiophenyl (e.g., thiophen-2-yl or thiophen-3-yl), oxazolyl (e.g., oxazol-2-yl, oxazol-4-yl, or oxazol-5-yl), isoxazolyl (e.g., isoxazol-3-yl, isoxazol-4-yl, or isoxazol-5-yl), thiazolyl, isothiazolyl, pyridinyl (e.g., pyridin-2-yl, pyridin-3-yl, or pyridin-4-yl), pyridazinyl, pyrimidinyl, pyrazinyl, 1H-indolyl, 2H-isoindolyl, indolizinyl (e.g., indolizin-1-yl or indolizin-2-yl), 1H-indazolyl, benzimidazolyl, benzofuranyl (e.g., benzofuran-2-yl, benzofuran-3-yl, benzofuran-4-yl, benzofuran-5-yl, benzofuran-6-yl, or benzofuran-7-yl), isobenzofuranyl, benzo[b]thiophenyl (e.g., benzo[b]thiophen-2-yl, benzo[b]thiophen-3-yl, benzo[b]thiophen-4-yl, benzo[b]thiophen-5-yl, benzo[b]thiophen-6-yl, or benzo[b]thiophen-7-yl), benzo[c]thiophenyl, quinolinyl, isoquinolinyl, quinoxalinyl, phthalazinyl, quinazolinyl, and cinnolinyl. If R¹ is -L¹-cycloalkyl [e.g., -L¹-(C₃₋₇ cycloalkyl)] wherein the cycloalkyl in said -L¹-cycloalkyl is optionally substituted with one or more groups R¹¹, then the cycloalkyl in said -L¹-cycloalkyl may be, e.g., selected from cyclopropyl, cyclobutyl, cyclopentyl, cyclohexyl, or cycloheptyl. Even more preferably, R¹ is selected from -L¹-phenyl, -L¹-(monocyclic 5- or 6-membered heteroaryl), or -L¹-(bicyclic 9- or 10-membered heteroaryl), wherein the phenyl in said -L¹-phenyl, the heteroaryl in said -L¹-(monocyclic 5- or 6-membered heteroaryl) and the heteroaryl in said -L¹-(bicyclic 9- or 10-membered heteroaryl) are each optionally substituted with one or more groups R¹¹. Yet even more preferably, R¹ is -L¹-phenyl, wherein the phenyl in said -L¹-phenyl is optionally substituted with one or more (e.g., one, two, or three) groups R¹¹.

Further examples of R¹ include any of the specific groups R¹ comprised in the compounds of formula (I) described in the examples section, particularly in any one of Examples 1 to 282.

L¹ is independently selected from a bond, —C(R^{L1})(R^{L1})—, —O—, —S—, —SO—, —SO₂—, —CO—, and —N(R^{L1})—, wherein each R^{L1} is independently hydrogen or C₁₋₅ alkyl, and further wherein two groups R^{L1} which are attached to the same carbon atom may also be mutually joined to form, together with the carbon atom that they are attached to, a cycloalkyl (e.g., a C₃₋₆ cycloalkyl) or a heterocycloalkyl (e.g., a 3 to 6-membered heterocycloalkyl).

Preferably, L¹ is independently selected from a bond, —CH₂—, —CH(C₁₋₅ alkyl)-, —C(C₁₋₅ alkyl)(C₁₋₅ alkyl)-, C₃₋₆ cycloalkyl-1,1-ene, —O—, —S—, —SO—, —SO₂—, —CO—, —NH—, and —N(C₁₋₅ alkyl)-. More preferably, L¹ is independently selected from a bond, —CH₂—, —CH(C₁₋₅ alkyl)-, —C(C₁₋₅ alkyl)(C₁₋₅ alkyl)-, and C₃₋₅ cycloalkyl-1,1-ene (e.g., cyclopropyl-1,1-ene). Even more preferably, L¹ is independently selected from a bond, —CH₂—, —CH(C₁₋₃ alkyl)-, and —C(C₁₋₃ alkyl)(C₁₋₃ alkyl)-. Yet even more preferably, L¹ is a bond.

In accordance with the above definition of L¹, it is particularly preferred that R¹ is phenyl which is optionally substituted with one or more (e.g., one, two, or three) groups R¹¹. If said phenyl is optionally substituted with one group R¹¹, it is preferred that said group R¹¹ is attached in meta or para position on the phenyl (preferably in para position), i.e., that R¹ is 3-R¹¹-phenyl or 4-R¹¹-phenyl (preferably 4-R¹¹-phenyl). If said phenyl is optionally substituted with two groups R¹¹, it is preferred that the two groups R¹¹ are attached in meta and para position, i.e., that R¹ is 3-R¹¹-4-R¹¹-phenyl. If said phenyl is optionally substituted with three groups R¹¹, it is preferred that two of the three groups R¹¹ are attached in meta position and one group R¹¹ is attached in para position, i.e., that R¹ is 3-R¹¹-4-R¹¹-5-R¹¹-phenyl. It is furthermore preferred that said phenyl is substituted with two or three (particularly with two) groups R¹¹. Accordingly, it is particularly preferred that R¹ is 3-R¹¹-4-R¹¹-phenyl or 3-R¹¹-4-R¹¹-5-R¹¹-phenyl, wherein each R¹¹ is independently selected from halogen (e.g., —F, —Cl, —Br, or —I), C₁₋₅ haloalkyl (e.g., —CF₃), and C₁₋₅ alkyl (e.g., —CH₃), even more preferably wherein each R¹¹ is independently selected from —F, —Cl, —CF₃, and —CH₃. Corresponding preferred examples of R¹ include 4-chloro-3-fluoro-phenyl, 3,4-dichloro-phenyl, 3,4-difluoro-phenyl, 3-chloro-4-fluoro-phenyl, 3-fluoro-4-trifluoromethyl-phenyl, 3-chloro-4-trifluoromethyl-phenyl, 3-fluoro-4-methyl-phenyl, 3-chloro-4-methyl-phenyl, 3,4,5-trifluoro-phenyl, or 4-chloro-3,5-difluoro-phenyl. Particularly preferred examples of R¹ are 4-chloro-3-fluoro-phenyl or 3,4-difluoro-phenyl.

Each R¹¹ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more (e.g., one, two or three) groups R^{Cyc}.

Preferably, each R¹¹ is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}. More preferably, each R¹¹ is independently selected from C₁₋₅ alkyl, C₂₋₅ alkenyl, C₂₋₅ alkynyl, halogen, C₁₋₅ haloalkyl, —O—(C₁₋₅ haloalkyl), —CN, —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more groups independently selected from C₁₋₅ alkyl, C₂₋₅ alkenyl, C₂₋₅ alkynyl, halogen, C₁₋₅ haloalkyl, —O—(C₁₋₅ haloalkyl), and —CN. Even more preferably, each R¹¹ is independently selected from C₁₋₅ alkyl, halogen, C₁₋₅ haloalkyl, —O—(C₁₋₅ haloalkyl), and —CN. Yet even more preferably, each R¹¹ is independently selected from halogen (e.g., —F, —Cl, —Br, or —I), C₁₋₅ haloalkyl (e.g., —CF₃), and C₁₋₅ alkyl (e.g., methyl). Still more preferably, each R¹¹ is independently halogen (particularly —F or —Cl) or C₁₋₅ haloalkyl (particularly —CF₃).

Each R¹² is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), carbocyclyl, heterocyclyl, and -L^{Z}-R^{Z}, wherein said carbocyclyl and said heterocyclyl are each optionally substituted with one or more (e.g., one, two or three) groups R^{Cyc}.

Preferably, each R¹² is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), and —SO₂—(C₁₋₅ alkyl). More preferably, each R¹² is independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN.

R^{2A} and R^{2B} are mutually joined to form, together with the carbon atom that they are attached to, a cycloalkyl, cycloalkenyl, heterocycloalkyl, or heterocycloalkenyl, wherein said cycloalkyl, said cycloalkenyl, said heterocycloalkyl or said heterocycloalkenyl is optionally substituted with one or more (e.g., one, two or three) groups R²¹; alternatively, R^{2A} and R^{2B} are each independently selected from C₁₋₅ alkyl, C₂₋₅ alkenyl, C₂₋₅ alkynyl, —(C₀₋₅ alkylene)-carbocyclyl, and —(C₀₋₅ alkylene)-heterocyclyl, wherein said alkyl, said alkenyl, said alkynyl, the alkylene group in said —(C₀₋₅ alkylene)-carbocyclyl, and the alkylene group in said —(C₀₋₅ alkylene)-heterocyclyl are each optionally substituted with one or more groups R²², wherein one or more —CH₂— units comprised in said alkyl, said alkenyl, said alkynyl, in the alkylene group in said —(C₀₋₅ alkylene)-carbocyclyl, or in the alkylene group in said —(C₀₋₅ alkylene)-heterocyclyl are each optionally replaced by a group independently selected from —O—, —NH—, —N(C₁₋₅ alkyl)-, —CO—, —S—, —SO—, and —SO₂—, and further wherein the carbocyclyl group in said —(C₀₋₅ alkylene)-carbocyclyl and the heterocyclyl group in said —(CO₅ alkylene)-heterocyclyl are each optionally substituted with one or more groups R^{Cyc}, wherein R^{2A} and a group R^{Y} (if present) may also be mutually joined to form, together with the ring atoms that they are attached to, a carbocyclyl or heterocyclyl, wherein said carbocyclyl or said heterocyclyl is optionally substituted with one or more (e.g., one, two or three) groups R^{Cyc}.

As explained above, R^{2A} and R^{2B} may be mutually joined to form, together with the carbon atom that they are attached to, a cycloalkyl, cycloalkenyl, heterocycloalkyl or heterocycloalkenyl, wherein said cycloalkyl, said cycloalkenyl, said heterocycloalkyl or said heterocycloalkenyl is optionally substituted with one or more groups R²¹. It will be understood that said cycloalkyl, said cycloalkenyl, said heterocycloalkyl or said heterocycloalkenyl forms a spirocyclic ring system together with the fused rings B and D. Said cycloalkyl (which is formed from R^{2A}, R^{2B} and the carbon atom carrying R^{2A} and R^{2B}) is preferably a monocyclic cycloalkyl, more preferably a C₃₋₇ cycloalkyl (e.g., cyclopropyl, cyclobutyl, cyclopentyl, cyclohexyl, or cycloheptyl); a particularly preferred example of said cycloalkyl is cyclopentyl. Said cycloalkenyl (which is formed from R^{2A}, R^{2B} and the carbon atom carrying R^{2A} and R^{2B}) is preferably a monocyclic cycloalkenyl, more preferably a C₄₇ cycloalkenyl (e.g., cyclobutenyl, cyclopentenyl, cyclohexenyl, or cyclohepentyl); a particularly preferred example of said cycloalkenyl is cyclobutenyl or cyclopentenyl. Said heterocycloalkyl (which is formed from R^{2A}, R^{2B} and the carbon atom carrying R^{2A} and R^{2B}) is preferably a monocyclic heterocycloalkyl, more preferably a 3- to 7-membered heterocycloalkyl (e.g., containing one or two ring heteroatoms selected independently from oxygen, sulfur and nitrogen, wherein all remaining ring atoms are carbon atoms), even more preferably a 4- to 7-membered heterocycloalkyl having one ring heteroatom selected from oxygen, sulfur and nitrogen (wherein all other ring atoms are carbon atoms); corresponding examples of said heterocycloalkyl include tetrahydrofuranyl (which may be attached, e.g., via the carbon ring atom in 3-position), tetrahydropyranyl (which may be attached, e.g., via the carbon ring atom in 4-position), tetrahydrothiophenyl (which may be attached, e.g., via the carbon ring atom in 3-position), thianyl (which may be attached, e.g., via the carbon ring atom in 4-position), pyrrolidinyl (which may be attached, e.g., via the carbon ring atom in 3-position), or piperidinyl (which may be attached, e.g., via the carbon ring atom in 4-position); a particularly preferred example of said heterocycloalkyl is tetrahydrofuranyl (which may be attached, e.g., via the carbon ring atom in 3-position). Said heterocycloalkenyl (which is formed from R^{2A}, R^{2B} and the carbon atom carrying R^{2A} and R^{2B}) is preferably a monocyclic heterocycloalkenyl, more preferably a 4- to 7-membered heterocycloalkenyl (e.g., containing one or two ring heteroatoms selected independently from oxygen, sulfur and nitrogen, wherein all remaining ring atoms are carbon atoms), even more preferably a 4- to 7-membered heterocycloalkenyl having one ring heteroatom selected from oxygen, sulfur and nitrogen (wherein all other ring atoms are carbon atoms). It is particularly preferred that R^{2A} and R^{2B} are mutually joined to form, together with the carbon atom that they are attached to, a cycloalkyl or heterocycloalkyl, wherein said cycloalkyl or said heterocycloalkyl is optionally substituted with one or more groups R²¹. Even more preferably, R^{2A} and R^{2B} are mutually joined to form, together with the carbon atom that they are attached to, a cyclopentyl or a tetrahydrofuranyl, wherein said cyclopentyl or said tetrahydrofuranyl is optionally substituted with one or more groups R²¹ (yet even more preferably, R^{2A} and R^{2B} are mutually joined to form, together with the carbon atom that they are attached to, a cyclopentyl which is optionally substituted with one or more groups R²¹); thus, in accordance with the above preferred definitions of ring B and ring D, it is particularly preferred that the compound of formula (I) has one of the following structures:

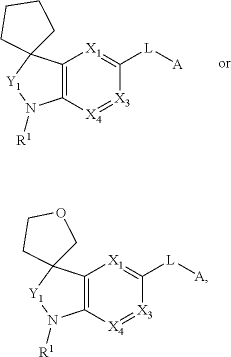

wherein the cyclopentyl ring and the tetrahydrofuran ring in the above-depicted formulae are each optionally substituted with one or more groups R²¹. In this case, in accordance with the above preferred definitions of ring B and ring D, it is even more preferred that the compound of formula (I) has one of the following structures:

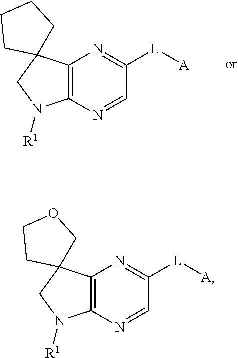

wherein the cyclopentyl ring and the tetrahydrofuran ring in the above-depicted formulae are each optionally substituted with one or more groups R²¹; yet even more preferably, the compound of formula (I) has the following structure:

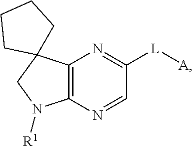

wherein the cyclopentyl ring in the above-depicted formula is optionally substituted with one or more groups R²¹.

Alternatively (i.e., if R^{2A} and R^{2B} are not mutually joined), the groups R^{2A} and R^{2B} are each independently selected from C₁₋₅ alkyl, C₂₋₅ alkenyl, C₂₋₅ alkynyl, —(C₀₋₅ alkylene)-carbocyclyl, and —(C₀₋₅ alkylene)-heterocyclyl, wherein said alkyl, said alkenyl, said alkynyl, the alkylene group in said —(C₀₋₅ alkylene)-carbocyclyl, and the alkylene group in said —(C₀₋₅ alkylene)-heterocyclyl are each optionally substituted with one or more (e.g., one, two, or three) groups R²², wherein one or more (e.g., one, two, or three) —CH₂— units comprised in said alkyl, said alkenyl, said alkynyl, in the alkylene group in said —(C₀₋₅ alkylene)-carbocyclyl, or in the alkylene group in said —(C₀₋₅ alkylene)-heterocyclyl are each optionally replaced by a group independently selected from —O—, —NH—, —N(C₁₋₅ alkyl)-, —CO—, —S—, —SO—, and —SO₂—, and further wherein the carbocyclyl group in said —(C₀₋₅ alkylene)-carbocyclyl and the heterocyclyl group in said —(C₀₋₅ alkylene)-heterocyclyl are each optionally substituted with one or more (e.g., one, two, or three) groups R^{Cyc}.

If R^{2A} and R^{2B} are not mutually joined, it is preferred that R^{2A} and R^{2B} are each independently selected from C₁₋₅ alkyl, C₂₋₅ alkenyl, C₂₋₅ alkynyl, —(C₀₋₅ alkylene)-carbocyclyl, and —(C₀₋₅ alkylene)-heterocyclyl, wherein said alkyl, said alkenyl, said alkynyl, the alkylene group in said —(C₀₋₅ alkylene)-carbocyclyl, and the alkylene group in said —(C₀₋₅ alkylene)-heterocyclyl are each optionally substituted with one or more (e.g., one, two, or three) groups independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN, wherein one or more (e.g., one, two, or three) —CH₂— units comprised in said alkyl, said alkenyl, said alkynyl, in the alkylene group in said —(C₀₋₅ alkylene)-carbocyclyl, or in the alkylene group in said —(C₀₋₅ alkylene)-heterocyclyl are each optionally replaced by a group independently selected from —O—, —NH—, —N(C₁₋₅ alkyl)-, —CO—, —S—, —SO—, and —SO₂—, and further wherein the carbocyclyl group in said —(C₀₋₅ alkylene)-carbocyclyl and the heterocyclyl group in said —(C₀₋₅ alkylene)-heterocyclyl are each optionally substituted with one or more (e.g., one, two, or three) groups R^{Cyc}. More preferably, R^{2A} and R^{2B} are each independently selected from C₁₋₅ alkyl, —(C₀₋₅ alkylene)-cycloalkyl, —(C₀₋₅ alkylene)-aryl, —(C₀₋₅ alkylene)-heterocycloalkyl, and —(C₀₋₅ alkylene)-heteroaryl, wherein said alkyl or the alkylene group in any of said —(C₀₋₅ alkylene)-cycloalkyl, said —(C₀₋₅ alkylene)-aryl, said —(C₀₋₅ alkylene)-heterocycloalkyl, or said —(C₀₋₅ alkylene)-heteroaryl is optionally substituted with one or more groups independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN, wherein one or more —CH₂— units comprised in said alkyl or in the alkylene group in any of said —(C₀₋₅ alkylene)-cycloalkyl, said —(C₀₋₅ alkylene)-aryl, said —(C₀₋₅ alkylene)-heterocycloalkyl, or said —(C₀₋₅ alkylene)-heteroaryl are each optionally replaced by a group independently selected from —O—, —NH—, —N(C₁₋₅ alkyl)-, —CO—, —S—, —SO—, and —SO₂—, and further wherein the cycloalkyl group in said —(C₀₋₅ alkylene)-cycloalkyl, the aryl group in said —(C₀₋₅ alkylene)-aryl, the heterocycloalkyl group in said —(C₀₋₅ alkylene)-heterocycloalkyl, and the heteroaryl group in said —(C₀₋₅ alkylene)-heteroaryl are each optionally substituted with one or more groups R^{Cyc}. Even more preferably, R^{2A} and R^{2B} are each independently selected from C₁₋₅ alkyl, —(C₀₋₅ alkylene)-cycloalkyl, —(C₀₋₅ alkylene)-aryl (e.g., —(C₀₋₅ alkylene)-phenyl, such as —CH₂-phenyl), —(C₀₋₅ alkylene)-heterocycloalkyl, and —(C₀₋₅ alkylene)-heteroaryl, wherein said alkyl or the alkylene group in any of said —(C₀₋₅ alkylene)-cycloalkyl, said —(C₀₋₅ alkylene)-aryl, said —(C₀₋₅ alkylene)-heterocycloalkyl, or said —(C₀₋₅ alkylene)-heteroaryl is optionally substituted with one or more groups independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN, and further wherein the cycloalkyl group in said —(C₀₋₅ alkylene)-cycloalkyl, the aryl group in said —(C₀₋₅ alkylene)-aryl, the heterocycloalkyl group in said —(C₀₋₅ alkylene)-heterocycloalkyl, and the heteroaryl group in said —(C₀₋₅ alkylene)-heteroaryl are each optionally substituted with one or more groups independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN. Yet even more preferably, R^{2A} and R^{2B} are each independently selected from C₁₋₅ alkyl, —(C₀₋₃ alkylene)-cycloalkyl (e.g., cyclopropyl, —CH₂-cyclopropyl, cyclobutyl, —CH₂-cyclobutyl, cyclopentyl, or —CH₂-cyclopentyl), —(C₀₋₃ alkylene)-heterocycloalkyl [e.g., oxetanyl (such as oxetan-2-yl or oxetan-3-yl), —CH₂-oxetanyl (such as oxetan-2-ylmethyl or oxetan-3-ylmethyl), tetrahydrofuranyl (such as tetrahydrofuran-3-yl), —CH₂-tetrahydrofuranyl (such as tetrahydrofuran-3-ylmethyl), tetrahydropyranyl (such as tetrahydropyran-4-yl), or —CH₂-tetrahydropyranyl (such astetrahydropyran-4-ylmethyl)], or —(C₀₋₃ alkylene)-heteroaryl [e.g., —CH₂-oxazolyl (such as oxazol-2-ylmethyl, oxazol-4-ylmethyl, or oxazol-5-ylmethyl), —CH₂-pyridinyl (such as pyridin-2-ylmethyl, pyridin-3-ylmethyl, or pyridin-4-ylmethyl), —C(—CH₃)(—CH₃)-pyridinyl (such as —C(—CH₃)(—CH₃)-(pyridin-2-yl)), —CH₂-pyrimidinyl (such as pyrimidin-2-ylmethyl), —CH₂-pyrazinyl (such as pyrazin-2-ylmethyl)], wherein said alkyl is optionally substituted with one or more groups independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN, and further wherein the cycloalkyl group in said —(C₀₋₃ alkylene)-cycloalkyl, the heterocycloalkyl group in said —(C₀₋₃ alkylene)-heterocycloalkyl and the heteroaryl group in said —(C₀₋₃ alkylene)-heteroaryl are each optionally substituted with one or more groups independently selected from C₁₋₅ alkyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN. Thus, for example, R^{2A} and R^{2B} may each be independently a C₁₋₅ alkyl which is optionally substituted with one or more groups independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN; a corresponding preferred example of R^{2A} and/or R^{2B} is C₁₋₅ alkyl (e.g., tert-butyl) substituted with one or two groups —O(C₁₋₅ alkyl), such as, e.g., —C(—CH₃)(—CH₃)—CH₂—O—CH₃, —C(—CH₃)(—CH₃)—CH₂—O—CH₂—CH₃, —CH(—CH₂—O—CH₃)(—CH₂—O—CH₃), —CH(—CH₃)—CH₂—O—CH₃, —CH₂CH₂—O—CH₃, or —CH₂CH₂—O—CH₂CH₃. A further example of R^{2A} and/or R^{2B} is —(C₀₋₃ alkylene)-phenyl which is optionally substituted with one or more groups independently selected from C₁₋₅ alkyl, halogen, C₁₋₅ haloalkyl, —O—(C₁₋₅ haloalkyl), and —CN; particularly 4-chloro-3-fluorophenyl or 4-chloro-3-fluorophenylmethyl. Thus, for example, R^{2A} may be 4-chloro-3-fluorophenyl and R^{2B} may be C₁₋₅ alkyl (e.g., methyl). Particularly preferred examples of each of R^{2A} and R^{2B} include methyl, ethyl, isopropyl, iso-butyl, sec-butyl (e.g., (S)-sec-butyl or (R)-sec-butyl), tert-butyl, cyclopropylmethyl, 1-methylcyclobutyl, 3-(methoxymethyl)cyclobutylmethyl, 2,2,2-trifluoroethyl, —C(—CH₃)(—CH₃)—CH₂—O—CH₃, —C(—CH₃)(—CH₃)—CH₂—O—CH₂—CH₃, —CH(—CH₃)—CH₂—O—CH₃ (e.g., (S)—CH(—CH₃)—CH₂—O—CH₃ or (R)—CH(—CH₃)—CH₂—O—CH₃), —CH₂CH₂—O—CH₃, —CH(—CH₂—O—CH₃)(—CH₂—O—CH₃), —CH₂-(cyclobut-1,3-diyl)-CH₂—O—CH₃, pyridin-2-ylmethyl, 6-methoxypyridin-2-ylmethyl, or 1-methyl-1-(pyridin-2-yl)ethyl. Still more preferably, R^{2A} and R^{2B} are each independently C₁₋₅ alkyl (e.g., methyl, ethyl, isopropyl, iso-butyl, sec-butyl, or tert-butyl). Thus, as an especially preferred example, R^{2A} and R^{2B} may each be methyl. It will be understood that for each of the general and preferred definitions of R^{2A} and R^{2B} described herein above, the groups R^{2A} and R^{2B} may be the same or different.

Moreover, as explained above, the group R^{2A} may also be mutually joined with a group R^{Y} (if present; preferably with a group R^{Y} that is attached to a ring atom directly adjacent to the carbon ring atom carrying R^{2A}) to form, together with the ring atoms that said groups R^{2A} and R^{Y} are attached to, a carbocyclyl or heterocyclyl, wherein said carbocyclyl or said heterocyclyl is optionally substituted with one or more (e.g., one, two or three) groups R^{Cyc}. In particular, the group R^{2A} may be mutually joined with a group R^{Y} (if present), wherein said group R^{Y} is attached to a ring atom directly adjacent to the carbon ring atom carrying R^{2A}, to form, together with the ring atoms that said groups R^{2A} and R^{Y} are attached to, a carbocyclyl or heterocyclyl, wherein said carbocyclyl or said heterocyclyl is optionally substituted with one or more groups R^{Cyc}; it will be understood that the corresponding carbocyclyl or heterocyclyl is then fused to ring B, which together with ring D results in a fused tricyclic ring system. The carbocyclyl formed from said groups R^{2A} and R^{Y} (and from the ring atoms that these groups R^{2A} and R^{Y} are attached to) may be, e.g., a cycloalkyl, a cycloalkenyl, or an aryl; preferably, said carbocyclyl is a cycloalkyl, such as, e.g., cyclopentyl or cyclohexyl. The heterocyclyl formed from said groups R^{2A} and R^{Y} (and from the ring atoms that these groups R^{2A} and R^{Y} are attached to) may be, e.g., a heterocycloalkyl, a heterocycloalkenyl, or a heteroaryl; preferably, said heterocyclyl is a heterocycloalkyl, such as, e.g., tetrahydrofuranyl (which may be attached, e.g., via the ring carbon atoms in positions 2 and 3). Accordingly, if R^{2A} and a group R^{Y} (preferably a group R^{Y} that is attached to a ring atom directly adjacent to the carbon ring atom carrying R^{2A}) are mutually joined, it is particularly preferred that they are mutually joined to form, together with the ring atoms that they are attached to, a cycloalkyl or heterocycloalkyl, wherein said cycloalkyl or said heterocycloalkyl is optionally substituted with one or more groups R^{Cyc}. It will be understood that if R^{2A} and a group R^{Y} are mutually joined, then the group R^{2B} has the same meaning as described herein above (see the general and preferred meanings of R^{2B} in the case that R^{2A} and R^{2B} are not mutually joined).

Each R²¹ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more (e.g., one, two, or three) groups R^{Cyc}.

Preferably, each R²¹ is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}. More preferably, each R²¹ is independently selected from C₁₋₅ alkyl, C₂₋₅ alkenyl, C₂₋₅ alkynyl, halogen, C₁₋₅ haloalkyl, —O—(C₁₋₅ haloalkyl), —CN, —(C₀₋₃ alkylene)-carbocyclyl, and —(C₀₋₃ alkylene)-heterocyclyl, wherein the carbocyclyl group in said —(C₀₋₃ alkylene)-carbocyclyl and the heterocyclyl group in said —(C₀₋₃ alkylene)-heterocyclyl are each optionally substituted with one or more groups independently selected from C₁₋₅ alkyl, C₂₋₅ alkenyl, C₂₋₅ alkynyl, halogen, C₁₋₅ haloalkyl, —O—(C₁₋₅ haloalkyl), and —CN. Even more preferably, each R²¹ is independently selected from C₁₋₅ alkyl (e.g., methyl), halogen (e.g., —F, —Cl, —Br, or -1), C₁₋₅ haloalkyl (e.g., —CF₃), —O—(C₁₋₅ haloalkyl) (e.g., —OCF₃), and —CN.

Each R²² is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), carbocyclyl, heterocyclyl, and -L^{Z}-R^{Z}, wherein said carbocyclyl and said heterocyclyl are each optionally substituted with one or more (e.g., one, two, or three) groups R^{Cyc}. Preferably, each R²² is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), and —SO₂—(C₁₋₅ alkyl). More preferably, each R²² is independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN.

Each R^{X} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more (e.g., one, two or three) groups R^{Cyc}.

Preferably, each R^{X} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(CO₃ alkylene)-carbocyclyl and the heterocyclyl group in said —(C₀₋₃ alkylene)-heterocyclyl are each optionally substituted with one or more groups R^{Cyc}. More preferably, each R^{X} is independently selected from C₁₋₅ alkyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), —CN, —(C₀₋₃ alkylene)-cycloalkyl (e.g., cyclopropyl), and —(C₀₋₃ alkylene)-heterocycloalkyl, wherein the cycloalkyl group in said —(C₀₋₃ alkylene)-cycloalkyl and the heterocycloalkyl group in said —(C₀₋₃ alkylene)-heterocycloalkyl are each optionally substituted with one or more groups R^{Cyc}.

Each R^{Y} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more (e.g., one, two or three) groups R^{Cyc};
wherein any two groups R^{Y} (if present) which are attached to the same ring carbon atom (i) may also be mutually joined to form, together with the carbon atom that they are attached to, a cycloalkyl or a heterocycloalkyl, wherein said cycloalkyl or said heterocycloalkyl is optionally substituted with one or more (e.g., one, two or three) groups R^{Cyc}, or (ii) may mutually form a group ═O.

Preferably, each R^{Y} is independently selected from C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-cycloalkyl (e.g., cyclopropyl), —(C₀₋₃ alkylene)-heteroaryl (e.g., pyridinyl; such as pyridin-2-yl), and —(C₀₋₃ alkylene)-heterocycloalkyl, wherein the aryl group in said —(C₀₋₃ alkylene)-aryl, the cycloalkyl group in said —(C₀₋₃ alkylene)-cycloalkyl, the heteroaryl group in said —(C₀₋₃ alkylene)-heteroaryl, and the heterocycloalkyl group in said —(CO₃ alkylene)-heterocycloalkyl are each optionally substituted with one or more groups R^{Cyc}; wherein any two groups R^{Y} (if present) which are attached to the same ring carbon atom (i) may also be mutually joined to form, together with the carbon atom that they are attached to, a cycloalkyl (e.g., a C₃₋₇ cycloalkyl) or a heterocycloalkyl (e.g., a 3- to 7-membered heterocycloalkyl), wherein said cycloalkyl or said heterocycloalkyl is optionally substituted with one or more groups R^{Cyc}, or (ii) may mutually form a group ═O. More preferably, each R^{Y} is independently selected from C₁₋₅ alkyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN. It is furthermore preferred that ring B is substituted with 0, 1, 2 or 3 groups R^{Y}, more preferably with 0, 1 or 2 groups R^{Y}, even more preferably with 0 or 1 group R^{Y}, yet even more preferably ring B is not substituted with any groups R^{Y}.

The group L is selected from —CO—, —SO— and —SO₂—. Preferably, L is —CO— or —SO₂—. More preferably, L is —CO—.

The group A is —N(—R^{N})—R^{N} or heterocyclyl, wherein said heterocyclyl is attached via a ring nitrogen atom to group L, and wherein said heterocyclyl is optionally substituted with one or more (e.g., one, two, three, or four) groups R^{A}.

Each R^{N} is independently selected from hydrogen, C₁₋₈ alkyl, C₂₋₈ alkenyl, C₂₋₈ alkynyl, —(C₀₋₈ alkylene)-OH, —(C₀₋₈ alkylene)-O(C₁₋₅ alkyl), —(C₀₋₈ alkylene)-SH, —(C₀₋₈ alkylene)-S(C₁₋₅ alkyl), —(C₁₋₈ alkylene)-NH₂, —(C₁₋₈ alkylene)-NH(C₁₋₅ alkyl), —(C₁₋₈ alkylene)-N(C₁₋₅ alkyl)(C₁₋₅ alkyl), —(C₁₋₈ alkylene)-halogen, —(C₁₋₈ alkylene)-C₁₋₅ haloalkyl, —(C₀₋₈ alkylene)-O—(C₁₋₈ haloalkyl), —(C₀₋₈ alkylene)-CN, —(C₀₋₈ alkylene)-CHO, —(C₀₋₈ alkylene)-CO—(C₁₋₅ alkyl), —(C₀₋₈ alkylene)-COOH, —(C₀₋₈ alkylene)-CO—O—(C₁₋₅ alkyl), —(C₀₋₈ alkylene)-O—CO—(C₁₋₅ alkyl), —(C₀₋₈ alkylene)-CO—NH₂, —(C₀₋₈ alkylene)-CO—NH(C₁₋₅ alkyl), —(C₀₋₈ alkylene)-CO—N(C₁₋₅ alkyl)(C₁₋₅ alkyl), —(C₁₋₈ alkylene)-NH—CO—(C₁₋₅ alkyl), —(C₁₋₈ alkylene)-N(C₁₋₅ alkyl)-CO—(C₁₋₅ alkyl), —(C₁₋₈ alkylene)-NH—COO(C₁₋₅ alkyl), —(C₁₋₈ alkylene)-N(C₁₋₅ alkyl)-COO(C₁₋₅ alkyl), —(C₀₋₈ alkylene)-O—CO—NH(C₁₋₅ alkyl), —(C₀₋₈ alkylene)-O—CO—N(C₁₋₅ alkyl)(C₁₋₅ alkyl), —(C₀₋₈ alkylene)-SO₂—NH₂, —(C₀₋₈ alkylene)-SO₂—NH(C₁₋₅ alkyl), —(C₀₋₈ alkylene)-SO₂—N(C₁₋₅ alkyl)(C₁₋₅ alkyl), —(C₁₋₈ alkylene)-NH—SO₂—(C₁₋₅ alkyl), —(C₁₋₈ alkylene)-N(C₁₋₅ alkyl)-SO₂—(C₁₋₅ alkyl), —(C₀₋₈ alkylene)-SO—(C₁₋₅ alkyl), —(C₀₋₈ alkylene)-SO₂—(C₁₋₅ alkyl), —(C₀₋₈ alkylene)-carbocyclyl, and —(C₀₋₈ alkylene)-heterocyclyl, wherein one or more (e.g., one, two, or three) —CH₂— units comprised in said C₁₋₈ alkyl, said C₂₋₈ alkenyl, said C₂₋₈ alkynyl, and in any of the aforementioned C₀₋₈ alkylene and C₁₋₈ alkylene groups are each optionally replaced by a group independently selected from —O—, —NH—, —N(C₁₋₅ alkyl)-, —CO—, —S—, —SO—, and —SO₂—, wherein the carbocyclyl group in said —(C₀₋₈ alkylene)-carbocyclyl and the heterocyclyl group in said —(C₀₋₈ alkylene)-heterocyclyl are each optionally substituted with one or more (e.g., one, two, or three) groups R^{Cyc}, and wherein at least one group R^{N} is not hydrogen.

Preferably, each R^{N} is independently selected from hydrogen, C₁₋₅ alkyl, C₂₋₅ alkenyl, C₂₋₅ alkynyl, —(C₀₋₅ alkylene)-OH, —(C₀₋₅ alkylene)-O(C₁₋₅ alkyl), —(C₀₋₅ alkylene)-SH, —(C₀₋₅ alkylene)-S(C₁₋₅ alkyl), —(C₁₋₅ alkylene)-NH₂, —(C₁₋₅ alkylene)-NH(C₁₋₅ alkyl), —(C₁₋₅ alkylene)-N(C₁₋₅ alkyl)(C₁₋₅ alkyl), —(C₁₋₅ alkylene)-halogen, —(C₁₋₅ alkylene)-C₁₋₅ haloalkyl, —(C₀₋₅ alkylene)-O—(C₁₋₅ haloalkyl), —(C₀₋₅ alkylene)-CN, —(C₀₋₅ alkylene)-CHO, —(C₀₋₅ alkylene)-CO—(C₁₋₅ alkyl), —(C₀₋₅ alkylene)-COOH, —(C₀₋₅ alkylene)-CO—O—(C₁₋₅ alkyl), —(C₀₋₅ alkylene)-O—CO—(C₁₋₅ alkyl), —(C₀₋₅ alkylene)-CO—NH₂, —(C₀₋₅ alkylene)-CO—NH(C₁₋₅ alkyl), —(C₀₋₅ alkylene)-CO—N(C₁₋₅ alkyl)(C₁₋₅ alkyl), —(C₁₋₅ alkylene)-NH—CO—(C₁₋₅ alkyl), —(C₁₋₅ alkylene)-N(C₁₋₅ alkyl)-CO—(C₁₋₅ alkyl), —(C₁₋₅ alkylene)-NH—COO(C₁₋₅ alkyl), —(C₁₋₅ alkylene)-N(C₁₋₅ alkyl)-COO(C₁₋₅ alkyl), —(C₀₋₅ alkylene)-O—CO—NH(C₁₋₅ alkyl), —(C₀₋₅ alkylene)-O—CO—N(C₁₋₅ alkyl)(C₁₋₅ alkyl), —(C₀₋₅ alkylene)-SO₂—NH₂, —(C₀₋₅ alkylene)-SO₂—NH(C₁₋₅ alkyl), —(C₀₋₅ alkylene)-SO₂—N(C₁₋₅ alkyl)(C₁₋₅ alkyl), —(C₁₋₅ alkylene)-NH—SO₂—(C₁₋₅ alkyl), —(C₁₋₅ alkylene)-N(C₁₋₅ alkyl)-SO₂—(C₁₋₅ alkyl), —(C₀₋₅ alkylene)-SO—(C₁₋₅ alkyl), —(C₀₋₅ alkylene)-SO₂—(C₁₋₅ alkyl), —(C₀₋₅ alkylene)-carbocyclyl, and —(C₀₋₅ alkylene)-heterocyclyl, wherein one or more (e.g., one, two, or three) —CH₂— units comprised in said C₁₋₅ alkyl, said C₂₋₅ alkenyl, said C₂₋₅ alkynyl, and in any of the aforementioned C₀₋₅ alkylene and C₁₋₅ alkylene groups are each optionally replaced by a group independently selected from —O—, —NH—, —N(C₁₋₅ alkyl)-, —CO—, —S—, —SO—, and —SO₂—, wherein the carbocyclyl group in said —(C₀₋₅ alkylene)-carbocyclyl and the heterocyclyl group in said —(C₀₋₅ alkylene)-heterocyclyl are each optionally substituted with one or more (e.g., one, two, or three) groups R^{Cyc}, and wherein at least one group R^{N} is not hydrogen.

As explained above, if group A is —N(—R^{N})_R^{N}, then at least one group R^{N} is not hydrogen. In particular, the group A may be, e.g., —NH—R^{N}, —N(C₁₋₅ alkyl)-R^{N}, or —N[—(C₁₋₅ alkylene)-O(C₁₋₅ alkyl)]-R^{N}, wherein R^{N} is selected from C₁₋₅ alkyl, C₂₋₅ alkenyl, C₂₋₅ alkynyl, —(C₀₋₅ alkylene)-OH, —(C₀₋₅ alkylene)-O(C₁₋₅ alkyl), —(C₀₋₅ alkylene)-SH, —(C₀₋₅ alkylene)-S(C₁₋₅ alkyl), —(C₁₋₅ alkylene)-NH₂, —(C₁₋₅ alkylene)-NH(C₁₋₅ alkyl), —(C₁₋₅ alkylene)-N(C₁₋₅ alkyl)(C₁₋₅ alkyl), —(C₁₋₅ alkylene)-halogen, —(C₁₋₅ alkylene)-C₁₋₅ haloalkyl, —(C₀₋₅ alkylene)-O—(C₁₋₅ haloalkyl), —(C₀₋₅ alkylene)-CN, —(C₀₋₅ alkylene)-CHO, —(C₀₋₅ alkylene)-CO—(C₁₋₅ alkyl), —(C₀₋₅ alkylene)-COOH, —(C₀₋₅ alkylene)-CO—O—(C₁₋₅ alkyl), —(C₀₋₅ alkylene)-O—CO—(C₁₋₅ alkyl), —(C₀₋₅ alkylene)-CO—NH₂, —(C₀₋₅ alkylene)-CO—NH(C₁₋₅ alkyl), —(C₀₋₅ alkylene)-CO—N(C₁₋₅ alkyl)(C₁₋₅ alkyl), —(C₁₋₅ alkylene)-NH—CO—(C₁₋₅ alkyl), —(C₁₋₅ alkylene)-N(C₁₋₅ alkyl)-CO—(C₁₋₅ alkyl), —(C₁₋₅ alkylene)-NH—COO(C₁₋₅ alkyl), —(C₁₋₅ alkylene)-N(C₁₋₅ alkyl)-COO(C₁₋₅ alkyl), —(C₀₋₅ alkylene)-O—CO—NH(C₁₋₅ alkyl), —(C₀₋₅ alkylene)-O—CO—N(C₁₋₅ alkyl)(C₁₋₅ alkyl), —(C₀₋₅ alkylene)-SO₂—NH₂, —(C₀₋₅ alkylene)-SO₂—NH(C₁₋₅ alkyl), —(C₀₋₅ alkylene)-SO₂—N(C₁₋₅ alkyl)(C₁₋₅ alkyl), —(C₁₋₅ alkylene)-NH—SO₂—(C₁₋₅ alkyl), —(C₁₋₅ alkylene)-N(C₁₋₅ alkyl)-SO₂—(C₁₋₅ alkyl), —(C₀₋₅ alkylene)-SO—(C₁₋₅ alkyl), —(C₀₋₅ alkylene)-SO₂—(C₁₋₅ alkyl), —(C₀₋₅ alkylene)-carbocyclyl, and —(C₀₋₅ alkylene)-heterocyclyl, wherein one or more (e.g., one, two, or three) —CH₂— units comprised in said C₁₋₅ alkyl, said C₂₋₅ alkenyl, said C₂₋₅ alkynyl, and in any of the aforementioned C₀₋₅ alkylene and C₁₋₅ alkylene groups are each optionally replaced by a group independently selected from —O—, —NH—, —N(C₁₋₅ alkyl)-, —CO—, —S—, —SO—, and —SO₂—, and further wherein the carbocyclyl group in said —(C₀₋₅ alkylene)-carbocyclyl and the heterocyclyl group in said —(C₀₋₅ alkylene)-heterocyclyl are each optionally substituted with one or more (e.g., one, two, or three) groups R^{Cyc}. Corresponding preferred examples of group A include —NH—C(—CH₃)(—CH₃)—CH₂—N(—CH₃)-(5-carboxy-4,6-dimethyl-pyridin-2-yl), —NH—C(—CH₃)(—CH₃)—CH₂—N(—CH₃)-(5-carboxy-pyridin-2-yl), —NH—C(—CH₃)(—CH₃)—CO—N(—CH₃)—CH₂—CO—NH₂, —NH—C(—CH₃)(—CH₃)—CO—N(—CH₃)(—CH₃), —NH—C(—CH₃)(—CH₃)—CO—NH—CH₃, —NH—CH(—CH₃)—CH₂—COOH, —NH—CH₂—CH₂—CH(—CH₃)—COOH, —NH—CH₂—CH₂—CH(—CH₃)—CH₂—COOH, —NH—CH₂—CH₂—CH(—CH₃)—CH₂—CO—NH₂, —NH—CH₂—CH₂—CH(—CH₃)—CH₂—CO—N(—CH₃)—CH₃, —NH-(1-(aminocarbonyl)cyclopropan-1-yl), —N(C₁₋₅ alkyl)-C(—CH₃)(—CH₃)—CH₂—N(—CH₃)-(5-carboxy-4,6-dimethyl-pyridin-2-yl), —N(C₁₋₅ alkyl)-C(—CH₃)(—CH₃)—CH₂—N(—CH₃)-(5-carboxy-pyridin-2-yl), —N(C₁₋₅ alkyl)-C(—CH₃)(—CH₃)—CO—N(—CH₃)—CH₂—CO—NH₂, —N(C₁₋₅ alkyl)-C(—CH₃)(—CH₃)—CO—N(—CH₃)(—CH₃), —N(C₁₋₅ alkyl)-C(—CH₃)(—CH₃)—CO—NH—CH₃, —N(C₁₋₅ alkyl)-CH(—CH₃)—CH₂—COOH, —N(C₁₋₅ alkyl)-CH₂—CH₂—CH(—CH₃)—COOH, —N(C₁₋₅ alkyl)-CH₂—CH₂—CH(—CH₃)—CH₂—COOH, —N(C₁₋₅ alkyl)-CH₂—CH₂—CH(—CH₃)—CH₂—CO—NH₂, —N(C₁₋₅ alkyl)- CH₂—CH₂—CH(—CH₃)—CH₂—CO—N(—CH₃)—CH₃, —N(C₁₋₅ alkyl)-(1-(aminocarbonyl)cyclopropan-1-yl), —N[—(C₁₋₅ alkylene)-O(C₁₋₅ alkyl)]-C(—CH₃)(—CH₃)—CH₂—N(—CH₃)-(5-carboxy-4,6-dimethyl-pyridin-2-yl), —N[—(C₁₋₅ alkylene)-O(C₁₋₅ alkyl)]-C(—CH₃)(—CH₃)—CH₂—N(—CH₃)-(5-carboxy-pyridin-2-yl), —N[—(C₁₋₅ alkylene)-O(C₁₋₅ alkyl)]-C(—CH₃)(—CH₃)—CO—N(—CH₃)—CH₂—CO—NH₂, —N[—(C₁₋₅ alkylene)-O(C₁₋₅ alkyl)]-C(—CH₃)(—CH₃)—CO—N(—CH₃)(—CH₃), —N[—(C₁₋₅ alkylene)-O(C₁₋₅ alkyl)]-C(—CH₃)(—CH₃)—CO—NH—CH₃, —N[—(C₁₋₅ alkylene)-O(C₁₋₅ alkyl)]-CH(—CH₃)—CH₂—COOH, —N[—(C₁₋₅ alkylene)-O(C₁₋₅ alkyl)]-CH₂—CH₂—CH(—CH₃)—COOH, —N[—(C₁₋₅ alkylene)-O(C₁₋₅ alkyl)]-CH₂—CH₂—CH(—CH₃)—CH₂—COOH, —N[—(C₁₋₅ alkylene)-O(C₁₋₅ alkyl)]—CH₂—CH₂—CH(—CH₃)—CH₂—CO—NH₂, —N[—(C₁₋₅ alkylene)-O(C₁₋₅ alkyl)]-CH₂—CH₂—CH(—CH₃)—CH₂—CO—N(—CH₃)—CH₃, or —N[—(C₁₋₅ alkylene)-O(C₁₋₅ alkyl)]-(1-(aminocarbonyl)cyclopropan-1-yl). Particularly preferred examples of group A include —N(—CH₃)—C(—CH₃)(—CH₃)—CH₂—N(—CH₃)-(5-carboxy-4,6-dimethyl-pyridin-2-yl), —N(—CH₃)—C(—CH₃)(—CH₃)—CH₂—N(—CH₃)-(5-carboxy-pyridin-2-yl), —N(—CH₂CH₂—O—CH₃)—C(—CH₃)(—CH₃)—CH₂—N(—CH₃)-(5-carboxy-4,6-dimethyl-pyridin-2-yl), —N(—CH₂CH₂—O—CH₃)—C(—CH₃)(—CH₃)—CH₂—N(—CH₃)-(5-carboxy-pyridin-2-yl), —N(—CH₃)—C(—CH₃)(—CH₃)—CO—N(—CH₃)—CH₂—CO—NH₂, —N(—CH₃)—C(—CH₃)(—CH₃)—CO—N(—CH₃)(—CH₃), —N(—CH₃)—C(—CH₃)(—CH₃)—CO—NH—CH₃, —N(—CH₃)—CH(—CH₃)—CH₂—COOH, —N(—CH₃)—CH₂—CH₂—CH(—CH₃)—COOH, —N(—CH₃)—CH₂—CH₂—CH(—CH₃)—CH₂—COOH, —N(—CH₃)—CH₂—CH₂—CH(—CH₃)—CH₂—CO—NH₂, —N(—CH₃)—CH₂—CH₂—CH(—CH₃)—CH₂—CO—N(—CH₃)—CH₃, or —NH-(1- (aminocarbonyl)cyclopropan-1-yl).

Preferably, the group A is heterocyclyl which is attached via a ring nitrogen atom to group L, and wherein said heterocyclyl is optionally substituted with one or more groups R^{A}. Said heterocyclyl may be, e.g., a 5 to 14 membered heterocyclyl. More preferably, group A is heterocycloalkyl or heterocycloalkenyl, wherein said heterocycloalkyl or said heterocycloalkenyl is attached via a ring nitrogen atom to group L, and wherein said heterocycloalkyl or said heterocycloalkenyl is optionally substituted with one or more groups R^{A}. Said heterocycloalkyl or said heterocycloalkenyl may be, e.g., a 5 to 14 membered heterocycloalkyl or a 5 to 14 membered heterocycloalkenyl. Even more preferably, group A is heterocycloalkyl which is attached via a ring nitrogen atom to group L, wherein said heterocycloalkyl is optionally substituted with one or more groups R^{A}. Said heterocycloalkyl is preferably a 5 to 11 membered heterocycloalkyl containing one nitrogen ring atom (through which the heterocycloalkyl is attached to group L) and optionally containing one or more (e.g., one, two, or three) further ring heteroatoms independently selected from nitrogen, oxygen and sulfur, wherein all remaining ring atoms are carbon atoms, wherein any nitrogen ring atom (if present) and/or any sulfur ring atom (if present) is optionally oxidized, and wherein one or more carbon ring atoms are optionally oxidized (i.e., to form an oxo group). More preferably, said heterocycloalkyl is a 5 to 7 membered (even more preferably a 6-membered) monocyclic heterocycloalkyl containing one nitrogen ring atom (through which the heterocycloalkyl is attached to group L) and optionally containing one or two further ring heteroatoms independently selected from nitrogen, oxygen and sulfur, wherein all remaining ring atoms are carbon atoms, wherein any nitrogen ring atom (if present) and/or any sulfur ring atom (if present) is optionally oxidized, and wherein one or more carbon ring atoms are optionally oxidized. Moreover, the heterocycloalkyl may contain a lactam function, i.e. the heterocycloalkyl may contain a second nitrogen ring atom (in addition to the first nitrogen ring atom through which the group A is attached to group L) which is adjacent to an oxidized carbon ring atom (C═O). A corresponding preferred example of group A is 3-oxopiperazin-1-yl which is optionally substituted with one or more (e.g., one, two, three, or four) groups R^{A}; a corresponding particularly preferred example of group A is 2,2-dimethyl-piperazin-3-on-1-yl (which may optionally be further substituted with one or more R^{A}). A further preferred example of group A is 4-(5-carboxypyridin-2-yl)piperazin-1-yl which is optionally substituted with one or more (e.g., one, two, three, or four) groups R^{A}; corresponding preferred examples of group A include 2,2-dimethyl-4-(5-carboxy-4,6-dimethyl-pyridin-2-yl)piperazin-1-yl or 2,2-dimethyl-4-(5-carboxy-pyridin-2-yl)piperazin-1-yl (each of which may optionally be further substituted with one or more R^{A}), particularly 2,2-dimethyl-4-(5-carboxy-4,6-dimethyl-pyridin-2-yl)piperazin-1-yl. A further preferred example of group A is 4-(5-carboxymethylpyridin-2-yl)piperazin-1-yl which is optionally substituted with one or more (e.g., one, two, three, or four) groups R^{A}; a corresponding preferred example of group A includes 2,2-dimethyl-4-(5-carboxymethylpyridin-2-yl)piperazin-1-yl (which may optionally be further substituted with one or more R^{A}). Further preferred examples of group A are 4-(4-carboxythiazol-2-yl)piperazin-1-yl or 4-(5-carboxythiazol-2-yl)piperazin-1-yl, which are each optionally substituted with one or more (e.g., one, two, three, or four) groups R^{A}; corresponding preferred examples of group A include 2,2-dimethyl-4-(4-carboxythiazol-2-yl)piperazin-1-yl or 2,2-dimethyl-4-(5-carboxythiazol-2-yl)piperazin-1-yl (each of which may optionally be further substituted with one or more R^{A}). A further preferred example of group A is 4-(carboxymethyl)piperidin-1-yl which is optionally substituted with one or more (e.g., one, two, three, or four) groups R^{A}; corresponding preferred examples of group A include 3-methoxy-4-(carboxymethyl)piperidin-1-yl, 3-methyl-4-(carboxymethyl)piperidin-1-yl, or 3-fluoro-4-(carboxymethyl)piperidin-1-yl (each of which may optionally be further substituted with one or more R^{A}).

Further examples of group A include any of the specific groups A comprised in the compounds of formula (I) described in the examples section, particularly in any one of Examples 1 to 282.

Each R^{A} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more (e.g., one, two or three) groups R^{Cyc}; and further wherein any two groups R^{A}, which are attached to the same carbon atom of group A, may also be mutually joined to form, together with the carbon atom that they are attached to, a cycloalkyl or a heterocycloalkyl, wherein said cycloalkyl or said heterocycloalkyl is optionally substituted with one or more (e.g., one, two or three) groups R^{Cyc}.

Preferably, each R^{A} is independently selected from C₁₋₅ alkyl, —(C₀₋₃ alkylene)-OH, —(C₀₋₃ alkylene)-O(C₁₋₅ alkyl), —(C₀₋₃ alkylene)-SH, —(C₀₋₃ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-aryl, —(C₀₋₃ alkylene)-cycloalkyl (e.g., cyclopropyl), —(C₀₋₃ alkylene)-heteroaryl (e.g., pyridinyl; such as pyridin-2-yl), and —(C₀₋₃ alkylene)-heterocycloalkyl, wherein the aryl group in said —(C₀₋₃ alkylene)-aryl, the cycloalkyl group in said —(C₀₋₃ alkylene)-cycloalkyl, the heteroaryl group in said —(C₀₋₃ alkylene)-heteroaryl, and the heterocycloalkyl group in said —(C₀₋₃ alkylene)-heterocycloalkyl are each optionally substituted with one or more groups R^{Cyc}; and further wherein any two groups R^{A}, which are attached to the same carbon atom of group A, may also be mutually joined to form, together with the carbon atom that they are attached to, a cycloalkyl (e.g., a C₃₋₇ cycloalkyl, such as cyclopropyl).

If group A is a heterocyclyl (as described herein above, including any of the corresponding preferred or exemplary cyclic groups A described herein; referred to as “ring A” in the following), it is particularly preferred that at least two substituents R^{A} are present, which are attached to the same carbon ring atom of ring A, and which are each independently a C₁₋₅ alkyl group or which are mutually joined to form, together with the carbon ring atom that they are attached to, a C₃₋₇ cycloalkyl group. Accordingly, it is particularly preferred that group A is a heterocycloalkyl (including any of the specific heterocycloalkyl groups described herein above) which is attached via a ring nitrogen atom to group L, wherein said heterocycloalkyl is either (i) substituted with two C₁₋₅ alkyl groups which are attached to the same ring carbon atom or is (ii) substituted with two substituents R^{A} which are attached to the same ring carbon atom and are mutually joined to form, together with the ring carbon atom that they are attached to, a C₃₋₇ cycloalkyl group (e.g. a cyclopropyl group), and wherein said heterocycloalkyl is optionally further substituted with one or more groups R^{A}. Even more preferably, group A is a heterocycloalkyl (including any of the specific heterocycloalkyl groups described herein above) which is attached via a ring nitrogen atom to group L, wherein said heterocycloalkyl is substituted with two C₁₋₅ alkyl groups which are attached to the same ring carbon atom, and wherein said heterocycloalkyl is optionally further substituted with one or more groups R^{A} (e.g., with one group R^{A} which is 5-carboxy-4,6-dimethyl-pyridin-2-yl). The two C₁₋₅ alkyl groups that are attached to the same ring carbon atom may be the same or different, and are preferably selected independently from methyl, ethyl, propyl and butyl; more preferably, the two C₁₋₅ alkyl groups that are attached to the same ring carbon atom are each methyl. The C₃₋₇ cycloalkyl group (which is formed from the two mutually joined substituents R^{A}) is preferably selected from cyclopropyl, cyclobutyl, cyclopentyl and cyclohexyl; more preferably, the C₃₋₇ cycloalkyl group is a cyclopropyl group. The position, i.e. the specific carbon ring atom of ring A, at which the two C₁₋₅ alkyl groups or the two mutually joined substituents R^{A} (which together form a C₃₋₇ cycloalkyl group) are attached is not particularly limited. For example, the two C₁₋₅ alkyl groups or the two mutually joined substituents R^{A} (which together form a C₃₋₇ cycloalkyl group) may be attached to a carbon ring atom (of ring A) which is (i) directly adjacent to the nitrogen ring atom through which ring A is attached to group L, or is (ii) separated by one ring atom from said nitrogen ring atom (through which ring A is attached to group L), or is (iii) separated by two ring atoms from said nitrogen ring atom (through which ring A is attached to group L). Corresponding preferred examples of ring A include 2,2-dimethyl-piperazin-1-yl, 3,3-dimethyl-piperazin-1-yl, 2,2-dimethyl-piperazin-3-on-1-yl, 2,2,4-trimethyl-piperazin-3-on-1-yl, 4-ethyl-2,2-dimethyl-piperazin-3-on-1-yl, spiro[piperazin-2,1′-cyclopropane]-1-yl, spiro[piperazin-3,1′-cyclopropane]-1-yl, 2,2-dimethyl-piperidin-1-yl, 3,3-dimethyl-piperidin-1-yl, 4,4-dimethyl-piperidin-1-yl, spiro[piperidin-2,1′-cyclopropane]-1-yl, spiro[piperidin-3,1′-cyclopropane]-1-yl, or spiro[piperidin-4,1′-cyclopropane]-1-yl, wherein the piperazinyl moiety, the piperazinonyl moiety or the piperidinyl moiety in each of the aforementioned groups is optionally further substituted with one or more (e.g., one or two) groups R^{A} (e.g., with one group R^{A} which is selected from 5-carboxypyridin-2-yl, 5-carboxy-4,6-dimethyl-pyridin-2-yl, 5-carboxymethyl-pyridin-2-yl, 5-carboxymethyl-4,6-dimethyl-pyridin-2-yl, 4-carboxythiazol-2-yl, and 5-carboxythiazol-2-yl). It is particularly preferred that the two C₁₋₅ alkyl groups or the two mutually joined substituents R^{A} (which together form a C₇ cycloalkyl group, preferably a cyclopropyl group) are attached to a carbon ring atom which is directly adjacent to the nitrogen ring atom through which ring A is attached to group L. A corresponding particularly preferred example of ring A is 2,2-dimethyl-piperazin-1-yl, wherein the piperazinyl group in said 2,2-dimethyl-piperazin-1-yl is optionally further substituted with one or more groups R^{A}; accordingly, ring A may be, e.g., 2,2-dimethyl-4-(5-carboxypyridin-2-yl)piperazin-1-yl, 2,2-dimethyl-4-(5-carboxy-4,6-dimethyl-pyridin-2-yl)piperazin-1-yl, 2,2-dimethyl-4-(5-carboxymethylpyridin-2-yl)piperazin-1-yl, 2,2-dimethyl-4-(5-carboxymethyl-4,6-dimethyl-pyridin-2-yl)piperazin-1-yl, 2,2-dimethyl-4-(4-carboxythiazol-2-yl)piperazin-1-yl, or 2,2-dimethyl-4-(5-carboxythiazol-2-yl)piperazin-1-yl. An even more preferred example of ring A is 2,2-dimethyl-4-(5-carboxy-4,6-dimethyl-pyridin-2-yl)piperazin-1-yl.

In accordance with the above, it is particularly preferred that group A is selected from any one of the following groups:

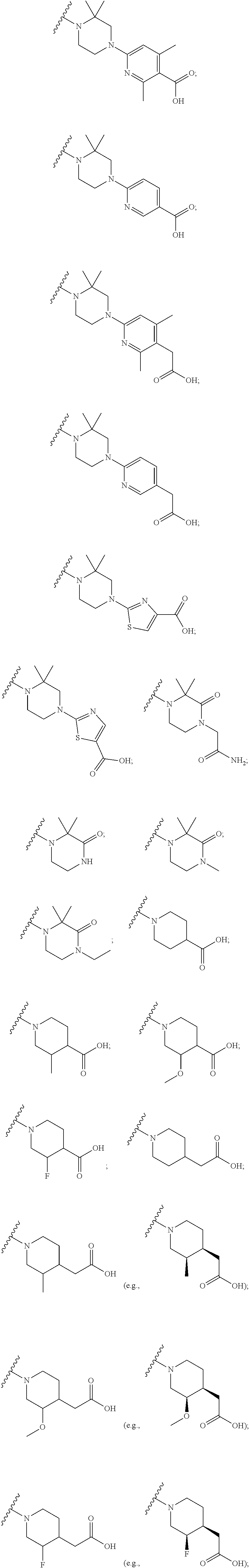

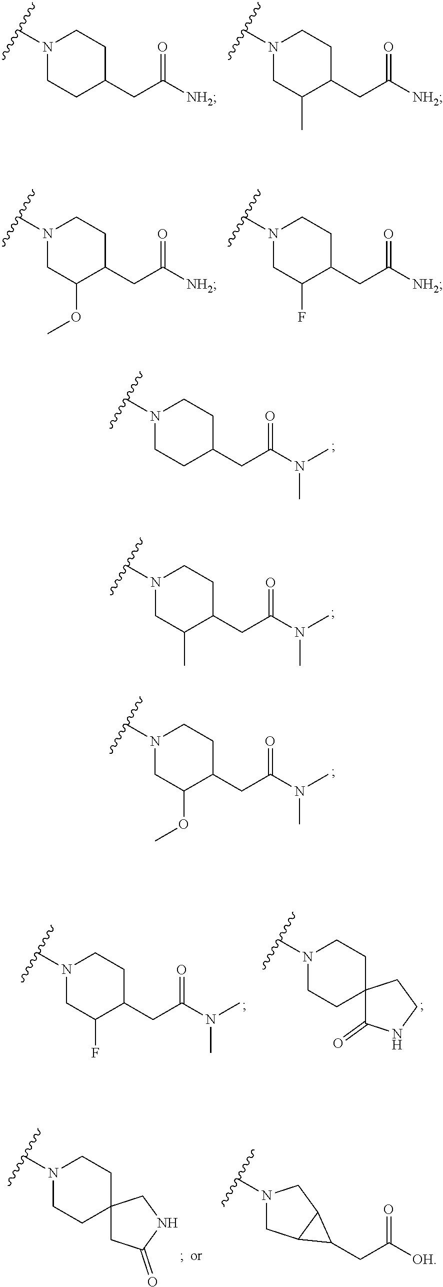

An especially preferred example of group A is 2,2-dimethyl-4-(5-carboxy-4,6-dimethyl-pyridin-2-yl)piperazin-1-yl:

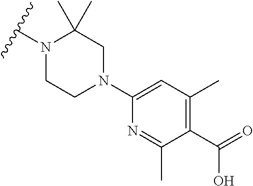 [SMILES: *N1CCN(c2cc(C)c(C(=O)O)c(C)n2)CC1(C)C]

The present invention particularly relates to compounds of formula (I) as well as pharmaceutically acceptable salts and solvates thereof, wherein group A is 2,2-dimethyl-4-(5-carboxy-4,6-dimethyl-pyridin-2-yl)piperazin-1-yl, and R^{2A} and R^{2B} are mutually joined to form, together with the carbon atom that they are attached to, a C₃₋₇ cycloalkyl (preferably a cyclopentyl) which is optionally substituted with one or more groups R²¹. The invention also specifically relates to compounds of formula (I) as well as pharmaceutically acceptable salts and solvates thereof, wherein group A is 2,2-dimethyl-4-(5-carboxy-4,6-dimethyl-pyridin-2-yl)piperazin-1-yl, and R^{2A} and R^{2B} are each independently C₁₋₅ alkyl (e.g., R^{2A} and R^{2B} may each be methyl).

A further especially preferred example of group A is 3-methoxy-4-(carboxymethyl)piperidin-1-yl:

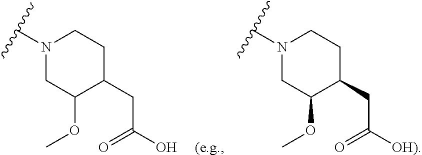

The invention particularly relates to compounds of formula (I) as well as pharmaceutically acceptable salts and solvates thereof, wherein group A is 3-methoxy-4-(carboxymethyl)piperidin-1-yl, and R^{2A} and R^{2B} are each independently C₁₋₅ alkyl (e.g., R^{2A} and R^{2B} may each be methyl).

A further especially preferred example of group A is 2,2-dimethyl-3-oxo-piperazin-1-yl:

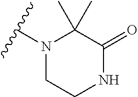 [SMILES: *N1CCNC(=O)C1(C)C]

The invention particularly relates to compounds of formula (I) as well as pharmaceutically acceptable salts and solvates thereof, wherein group A is 2,2-dimethyl-3-oxo-piperazin-1-yl, and R^{2A} and R^{2B} are each independently C₁₋₅ alkyl (e.g., R^{2A} and R^{2B} may each be methyl).

Each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), —P(═O)(—OH)(—OH), —P(═O)(—OH)(—O—C₁₋₅ alkyl), —P(═O)(—O—C₁₋₅ alkyl)(—O—C₁₋₅ alkyl), —(C₀₋₃ alkylene)-cycloalkyl, —(C₀₋₃ alkylene)-heterocycloalkyl, and -L^{Z}-R^{Z}.

Preferably, each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), —(C₀₋₃ alkylene)-cycloalkyl, —(C₀₋₃ alkylene)-heterocycloalkyl, and -L^{Z}-R^{Z}. More preferably, each R^{Cyc} is independently selected from C₁₋₅ alkyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN.

Each L^{Z} is independently selected from a covalent bond, C₁₇ alkylene, C₂₇ alkenylene, and C₂₇ alkynylene, wherein said alkylene, said alkenylene and said alkynylene are each optionally substituted with one or more (e.g., one, two, or three) groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more (e.g., one, two, or three) —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—.

Preferably, each L^{Z} is independently selected from a covalent bond, C₁₋₅ alkylene, C₂₋₅ alkenylene, and C₂₋₅ alkynylene, wherein said alkylene, said alkenylene and said alkynylene are each optionally substituted with one or more (e.g., one, two, or three) groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more (e.g., one, two, or three) —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—.

Each R^{Z} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, and heterocycloalkyl, wherein said aryl, said heteroaryl, said cycloalkyl, and said heterocycloalkyl are each optionally substituted with one or more (e.g., one, two or three) groups independently selected from C₁₋₅ alkyl, C₂₋₅ alkenyl, C₂₋₅ alkynyl, halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), carbocyclyl, and heterocyclyl, wherein said carbocyclyl and said heterocyclyl are each optionally substituted with one or more groups independently selected from C₁₋₅ alkyl, C₂₋₅ alkenyl, C₂₋₅ alkynyl, halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), and —SO₂—(C₁₋₅ alkyl).

Preferably, each R^{Z} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C_{1- 5} alkyl), aryl, heteroaryl, cycloalkyl, and heterocycloalkyl, wherein said aryl, said heteroaryl, said cycloalkyl, and said heterocycloalkyl are each optionally substituted with one or more (e.g., one, two or three) groups independently selected from C₁₋₅ alkyl, C₂₋₅ alkenyl, C₂₋₅ alkynyl, halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl).

It is preferred that at least one (more preferably all) of the following conditions apply to the compounds of formula (I):
if ring B is a pyrrolidinyl ring, ring D is a pyridinyl ring, R^{2A} and R^{2B} are mutually joined to form, together with the carbon atom that they are attached to, a cyclopropyl, L is —CO—, and group A is morpholin-4-yl, then R¹ is not 5-R¹¹-pyrimidin-2-yl or acetyl; and/or
if ring B and ring D together are a 2-oxo-2,3-dihydro-1H-pyrrolo[2,3-b]pyridinyl ring, a 2-oxo-2,3-dihydro-1H-pyrrolo[2,3-c]pyridinyl ring or a 6-oxo-6,7-dihydro-5H-pyrrolo[2,3-c]pyridazinyl ring, R^{2A} and R^{2B} are each methyl, R¹ is phenyl which is optionally substituted with one or more groups R¹¹, L is —CO—, and group A is —NH—R^{N}, then R^{N} is not a heterocycloalkyl which comprises one oxidized sulfur ring atom, in which all other ring atoms are carbon atoms, and which is substituted with a methyl group; and/or
if ring B and ring D together are a 3-R^{X}-4-oxo-4,5,6,7-tetrahydropyrazolo[1,5-a]pyrazinyl ring, R^{X} is —OH, one of R^{2A} and R^{2B} is methyl, the other one of R^{2A} and R^{2B} is —CON(—CH₃)₂, R¹ is methyl, L is —CO—, and group A is —NH—R^{N}, then R^{N} is not 4-fluorobenzyl; and/or
if ring B and ring D together are a 2-oxo-2,3-dihydro-1H-pyrrolo[3,2-b]pyridine ring, a 2-oxo-2,3-dihydro-1H-pyrrolo[2,3-b]pyridine ring or a 2-oxo-2,3-dihydro-1H-pyrrolo[3,2-c]pyridine, L is —CO—, group A is —NH—R^{N}, and R¹ is —CH₂-phenyl or —CH₂-pyridinyl, wherein the phenyl in said —CH₂-phenyl and the pyridinyl in said —CH₂-pyridinyl are each optionally substituted with one or more groups R¹¹, then R^{2A} and R^{2B} are not methyl.

Thus, as explained above, it is preferred that the following proviso applies to the compounds of formula (I): If ring B is a pyrrolidinyl ring, ring D is a pyridinyl ring, R^{2A} and R^{2B} are mutually joined to form, together with the carbon atom that they are attached to, a cyclopropyl, L is —CO—, and group A is morpholin-4-yl, then R¹ is not 5-R¹¹-pyrimidin-2-yl or acetyl. Accordingly, if B is a pyrrolidinyl ring, if D is a pyridinyl ring, if R^{2A} and R^{2B} are mutually joined to form (together with the carbon atom that they are attached to) a cyclopropyl, if L is —CO—, and if group A is morpholin-4-yl, then it is preferred that the group R¹ is not 5-R¹¹-pyrimidin-2-yl or acetyl (i.e., it is preferred that R¹ is not a pyrimidin-2-yl group which carries one substituent R¹¹ in the 5-position of the pyrimidine ring, and that R¹ is not acetyl). More preferably, if ring B is a pyrrolidinyl ring, ring D is a pyridinyl ring, L is —CO—, and group A is morpholin-4-yl, then R¹ is not 5-R¹¹-pyrimidin-2-yl or acetyl. Even more preferably, if ring B is a pyrrolidinyl ring, ring D is a pyridinyl ring, and L is —CO—, then R¹ is not 5-R¹¹-pyrimidin-2-yl or acetyl. Even more preferably, if ring B is a pyrrolidinyl ring and L is —CO—, then R¹ is not 5-R¹¹-pyrimidin-2-yl or acetyl. Even more preferably, if ring B is a pyrrolidinyl ring, then R¹ is not 5-R¹¹-pyrimidin-2-yl or acetyl. Yet even more preferably, R¹ is not 5-R¹¹-pyrimidin-2-yl (i.e., R¹ is a group different from 5-R¹¹-pyrimidin-2-yl) and/or R¹ is not acetyl. Still more preferably, R¹ is not 5-R¹¹-pyrimidin-2-yl and is not acetyl.

Moreover, as explained above, it is preferred that the following proviso applies to the compounds of formula (I): If ring B and ring D together are a 2-oxo-2,3-dihydro-1H-pyrrolo[2,3-b]pyridinyl ring, a 2-oxo-2,3-dihydro-1H-pyrrolo[2,3-c]pyridinyl ring or a 6-oxo-6,7-dihydro-5H-pyrrolo[2,3-c]pyridazinyl ring, R^{2A} and R^{2B} are each methyl, R¹ is phenyl which is optionally substituted with one or more groups R¹¹, L is —CO—, and group A is —NH—R^{N}, then R^{N} is not a heterocycloalkyl which comprises one oxidized sulfur ring atom, in which all other ring atoms are carbon atoms, and which is substituted with a methyl group. Accordingly, if ring B and ring D are fused to form a 2-oxo-2,3-dihydro-1H-pyrrolo[2,3-b]pyridinyl ring, a 2-oxo-2,3-dihydro-1H-pyrrolo[2,3-c]pyridinyl ring or a 6-oxo-6,7-dihydro-5H-pyrrolo[2,3-c]pyridazinyl ring, if R^{2A} and R^{2B} are each methyl, if R¹ is phenyl which is optionally substituted with one or more groups R¹¹, if L is —CO—, if group A is —N(—R^{N})—R^{N}, and if one group R^{N} is hydrogen, then it is preferred that the other group R^{N} is not a heterocycloalkyl which comprises one oxidized sulfur ring atom (particularly a ring atom —S(═O)₂—), in which all other ring atoms are carbon atoms, and which is substituted with a methyl group. More preferably, if ring B and ring D together are a 2-oxo-2,3-dihydro-1H-pyrrolo[2,3-b]pyridinyl ring, a 2-oxo-2,3-dihydro-1H-pyrrolo[2,3-c]pyridinyl ring or a 6-oxo-6,7-dihydro-5H-pyrrolo[2,3-c]pyridazinyl ring, R^{2A} and R^{2B} are each methyl, L is —CO—, and group A is —NH—R^{N}, then R^{N} is not a heterocycloalkyl which comprises one oxidized sulfur ring atom, in which all other ring atoms are carbon atoms, and which is substituted with a methyl group. Even more preferably, if ring B and ring D together are a 2-oxo-2,3-dihydro-1H-pyrrolo[2,3-b]pyridinyl ring, a 2-oxo-2,3-dihydro-1H-pyrrolo[2,3-c]pyridinyl ring or a 6-oxo-6,7-dihydro-5H-pyrrolo[2,3-c]pyridazinyl ring, L is —CO—, and group A is —NH—R^{N}, then R^{N} is not a heterocycloalkyl which comprises one oxidized sulfur ring atom, in which all other ring atoms are carbon atoms, and which is substituted with a methyl group. Even more preferably, if ring B is a 2-oxopyrrolidinyl ring, ring D is a pyridinyl ring or a pyridazinyl ring, L is —CO—, and group A is —NH—R^{N}, then R^{N} is not a heterocycloalkyl which comprises one oxidized sulfur ring atom, in which all other ring atoms are carbon atoms, and which is substituted with a methyl group. Even more preferably, if ring B is a 2-oxopyrrolidinyl ring and group A is —NH—R^{N}, then R^{N} is not a heterocycloalkyl which comprises one oxidized sulfur ring atom, in which all other ring atoms are carbon atoms, and which is substituted with a methyl group. Yet even more preferably, group A is not —NH—R^{N}, wherein R^{N} is a heterocycloalkyl which comprises one oxidized sulfur ring atom, in which all other ring atoms are carbon atoms, and which is substituted with a methyl group. Still more preferably, group A is not —NH—R^{N}, wherein R^{N} is heterocycloalkyl optionally substituted with one or more groups R^{Cyc}.

Furthermore, as explained above, it is preferred that the following proviso applies to the compounds of formula (I): If ring B and ring D together are a 3-R^{X}-4-oxo-4,5,6,7-tetrahydropyrazolo[1,5-a]pyrazinyl ring, R^{X} is —OH, one of R^{2A} and R^{2B} is methyl, the other one of R^{2A} and R^{2B} is —CON(—CH₃)₂, R¹ is methyl, L is —CO—, and group A is —NH—R^{N}, then R^{N} is not 4-fluorobenzyl. Accordingly, if ring B and ring D together are a 4-oxo-4,5,6,7-tetrahydropyrazolo[1,5-a]pyrazinyl ring which is substituted in position 3 with one group R^{X} which is —OH, if one of R^{2A} and R^{2B} is methyl, if the other one of R^{2A} and R^{2B} is —CON(—CH₃)CH₃, if R¹ is methyl, if L is —CO—, and if group A is —NH—R^{N}, then it is preferred that R^{N} is not 4-fluorobenzyl (i.e., that R^{N} is not a group —CH₂-(4-fluorophenyl)). More preferably, if ring B and ring D together are a 3-R^{X}-4-oxo-4,5,6,7-tetrahydropyrazolo[1,5-a]pyrazinyl ring, and if L is —CO—, then R^{X} is not —OH. Even more preferably, if ring D is a pyrazolyl ring which is optionally substituted with one or more groups R^{X}, and if L is —CO—, then said one or more groups R^{X} are not —OH.

As also explained above, it is preferred that the following proviso applies to the compounds of formula (I): If ring B and ring D together are a 2-oxo-2,3-dihydro-1H-pyrrolo[3,2-b]pyridine ring, a 2-oxo-2,3-dihydro-1H-pyrrolo[2,3-b]pyridine ring or a 2-oxo-2,3-dihydro-1H-pyrrolo[3,2-c]pyridine, L is —CO—, group A is —NH—R^{N}, and R¹ is —CH₂-phenyl or —CH₂-pyridinyl, wherein the phenyl in said —CH₂-phenyl and the pyridinyl in said —CH₂-pyridinyl are each optionally substituted with one or more groups R¹¹, then R^{2A} and R^{2B} are not methyl. More preferably, if ring B and ring D together are a 2-oxo-2,3-dihydro-1H-pyrrolo[3,2-b]pyridine ring, a 2-oxo-2,3-dihydro-1H-pyrrolo[2,3-b]pyridine ring or a 2-oxo-2,3-dihydro-1H-pyrrolo[3,2-c]pyridine, L is —CO—, and group A is —NH—R^{N}, then R^{2A} and R^{2B} are not methyl. Even more preferably, if ring B and ring D together are a 2-oxo-2,3-dihydro-1H-pyrrolo[3,2-b]pyridine ring, a 2-oxo-2,3-dihydro-1H-pyrrolo[2,3-b]pyridine ring, a 2-oxo-2,3-dihydro-1H-pyrrolo[3,2-c]pyridine or a 2-R^{Y}-2,3-dihydro-1H-pyrrolo[2,3-b]pyridine ring, L is —CO—, and group A is —NH—R^{N}, then R^{2A} and R^{2B} are not methyl. Yet even more preferably, if ring B is a 2-oxopyrrolidinyl ring or a 2-R^{Y}-pyrrolidinyl ring, ring D is a pyridinyl ring, L is —CO—, and group A is —NH—R^{N}, then R^{2A} and R^{2B} are not methyl.

Further to the above, it is preferred that the compound 1-(tert-butyl)-6-((4-fluorophenyl)carbamoyl)-2-oxo-1,2-dihydrospiro[pyrido[2,3-b][1,4]oxazine-3,3′-pyrrolidine] is excluded from formula (I). More preferably, if ring B is a non-aromatic 4- to 8-membered heterocyclic ring comprising a —C(═O)— ring atom adjacent to the ring carbon atom carrying R^{2A} and R^{2B}, wherein said ring B is optionally substituted with one or more groups R^{Y}, and if R^{2A} and R^{2B} are mutually joined to form, together with the carbon atom that they are attached to, a pyrrolidin-3-yl group which is optionally substituted with one or more groups R²¹, then R¹ is not tert-butyl. Even more preferably, if ring B is a non-aromatic 4- to 8-membered heterocyclic ring comprising a —C(═O)— ring atom adjacent to the ring carbon atom carrying R^{2A} and R^{2B}, wherein said ring B is optionally substituted with one or more groups R^{Y}, and if R^{2A} and R^{2B} are mutually joined to form, together with the carbon atom that they are attached to, a heterocycloalkyl which is optionally substituted with one or more groups R²¹, then said heterocycloalkyl is not pyrrolidin-3-yl.

It is particularly preferred that the compound of formula (I) is any one of the specific compounds of formula (I) described in the examples section of this specification, including any one of Examples 1 to 282 described further below, either in non-salt form and/or non-solvated form, or as a pharmaceutically acceptable salt or solvate of the respective compound.

Accordingly, it is particularly preferred that the compound of formula (I) is selected from:
1′-(4-chloro-3-fluorophenyl)-5′-(2,2-dimethyl-3-oxopiperazine-1-carbonyl)-2,3,5,6-tetrahydrospiro[pyran-4,3′-pyrrolo[3,2-b]pyridin]-2′(1′H)-one;
1′-(4-chloro-3-fluorophenyl)-5′-(2,2-dimethyl-3-oxopiperazine-1-carbonyl)spiro[cyclopentane-1,3′-pyrrolo[3,2-b]pyridin]-2′(1′H)-one;
4-(1′-(4-chloro-3-fluorophenyl)-1′,2′-dihydrospiro[cyclopentane-1,3′-pyrrolo[3,2-b]pyridine]-5′-carbonyl)-3,3-dimethylpiperazin-2-one;
8-(1′-(4-chloro-3-fluorophenyl)-1′,2′-dihydrospiro[cyclopentane-1,3′-pyrrolo[3,2-b]pyridine]-5′-carbonyl)-1,3,8-triazaspiro[4.5]decane-2,4-dione;
methyl 1-(1′-(4-chloro-3-fluorophenyl)-1′,2′-dihydrospiro[cyclopentane-1,3′-pyrrolo[3,2-b]pyridine]-5′-carbonyl)-3,3-dimethylpiperidine-4-carboxylate;
1-(1′-(4-chloro-3-fluorophenyl)-1′,2′-dihydrospiro[cyclopentane-1,3′-pyrrolo[3,2-b]pyridine]-5′-carbonyl)-3,3-dimethylpiperidine-4-carboxylic acid;
methyl 6-(4-(1′-(4-chloro-3-fluorophenyl)-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
6-(4-(1′-(4-chloro-3-fluorophenyl)-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
4-(1′-(3-chlorophenyl)-1′,2′-dihydrospiro[cyclopentane-1,3′-pyrrolo[3,2-b]pyridine]-5′-carbonyl)-3,3-dimethylpiperazin-2-one;
4-(1′-(2-chlorophenyl)-1′,2′-dihydrospiro[cyclopentane-1,3′-pyrrolo[3,2-b]pyridine]-5′-carbonyl)-3,3-dimethylpiperazin-2-one:
4-(3,3-dimethyl-1-(thiophen-3-yl)-2,3-dihydro-1H-pyrrolo[3,2-b]pyridine-5-carbonyl)-3,3-dimethylpiperazin-2-one;
4-(1-(3,4-difluorophenyl)-3,3-dimethyl-2,3-dihydro-1H-pyrrolo[3,2-b]pyridine-5-carbonyl)-3,3-dimethylpiperazin-2-one;
methyl 6-(4-(1-(3,4-difluorophenyl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(3,4-difluorophenyl)-3,3-dimethyl-2,3-dihydro-1H-pyrrolo[3,2-b]pyridine-5-carbonyl)-3,3-dimethylpiperazin-1-yl)-2,4-dimethylnicotinic acid;
4-(1-(4-fluorophenyl)-3,3-dimethyl-2,3-dihydro-1H-pyrrolo[3,2-b]pyridine-5-carbonyl)-3,3-dimethylpiperazin-2-one;
methyl 6-(4-(1-(3-fluorophenyl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(3-fluorophenyl)-3,3-dimethyl-2,3-dihydro-1H-pyrrolo[3,2-b]pyridine-5-carbonyl)-3,3-dimethylpiperazin-1-yl)-2,4-dimethylnicotinic acid;
4-(1-(4-chloro-3-fluorophenyl)-3,3-dimethyl-2,3-dihydro-1H-pyrrolo[3,2-b]pyridine-5-carbonyl)-3,3-dimethylpiperazin-2-one;
4-(1-(4-chloro-3-fluorophenyl)-3,3-dimethyl-2,3-dihydro-1H-pyrrolo[3,2-b]pyridine-5-carbonyl)-1-ethyl-3,3-dimethylpiperazin-2-one;
(1-(4-chloro-3-fluorophenyl)-3,3-dimethyl-2,3-dihydro-1H-pyrrolo[3,2-b]pyridin-5-yl)(3,3-dimethylmorpholino)methanone;
tert-butyl 4-(1-(4-chloro-3-fluorophenyl)-3,3-dimethyl-2,3-dihydro-1H-pyrrolo[3,2-b]pyridine-5-carbonyl)-3,3-dimethylpiperazine-1-carboxylate;
(1-(4-chloro-3-fluorophenyl)-3,3-dimethyl-2,3-dihydro-1H-pyrrolo[3,2-b]pyridin-5-yl)(2,2-dimethylpiperazin-1-yl)methanone;
(1-(4-chloro-3-fluorophenyl)-3,3-dimethyl-2,3-dihydro-1H-pyrrolo[3,2-b]pyridin-5-yl)(4-ethyl-2,2-dimethylpiperazin-1-yl)methanone;
1-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)ethan-1-one;
ethyl 4-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-oxobutanoate;
4-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-oxobutanoic acid;
ethyl 5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-5-oxopentanoate;
5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-5-oxopentanoic acid;
8-(1-(4-chloro-3-fluorophenyl)-3,3-dimethyl-2,3-dihydro-1H-pyrrolo[3,2-b]pyridine-5-carbonyl)-1-methyl-1,3,8-triazaspiro[4.5]decan-4-one;
2-(3-(1-(4-chloro-3-fluorophenyl)-3,3-dimethyl-2,3-dihydro-1H-pyrrolo[3,2-b]pyridine-5-carbonyl)-3-azabicyclo[3.1.0]hexan-6-yl)acetic acid;
methyl 2-(3-(1-(4-chloro-3-fluorophenyl)-3,3-dimethyl-2,3-dihydro-1H-pyrrolo[3,2-b]pyridine-5-carbonyl)-3-azabicyclo[3.1.0]hexan-6-yl)acetate;
methyl 2-((1R,5S,6S)-3-(1-(4-chloro-3-fluorophenyl)-3,3-dimethyl-2,3-dihydro-1H-pyrrolo[3,2-b]pyridine-5-carbonyl)-3-azabicyclo[3.1.0]hexan-6-yl)acetate;
2-((1R,5S,6S)-3-(1-(4-chloro-3-fluorophenyl)-3,3-dimethyl-2,3-dihydro-1H-pyrrolo[3,2-b]pyridine-5-carbonyl)-3-azabicyclo[3.1.0]hexan-6-yl)acetic acid;
ethyl1-(1-(4-chloro-3-fluorophenyl)-3,3-dimethyl-2,3-dihydro-1H-pyrrolo[3,2-b]pyridine-5-carbonyl)piperidine-4-carboxylate;
1-(1-(4-chloro-3-fluorophenyl)-3,3-dimethyl-2,3-dihydro-1H-pyrrolo[3,2-b]pyridine-5-carbonyl)piperidine-4-carboxylic acid;
methyl 2-(1-(1-(4-chloro-3-fluorophenyl)-3,3-dimethyl-2,3-dihydro-1H-pyrrolo[3,2-b]pyridine-5-carbonyl)piperidin-4-yl)acetate;
2-(1-(1-(4-chloro-3-fluorophenyl)-3,3-dimethyl-2,3-dihydro-1H-pyrrolo[3,2-b]pyridine-5-carbonyl)piperidin-4-yl)acetic acid;
2-(1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetic acid;
methyl 2-(1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetate;
methyl 2-((3R,4S)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetate;
2-((3R,4S)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetic acid;
ethyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylpyrimidine-5-carboxylate;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylpyrimidine-5-carboxylic acid;
ethyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyrimidine-5-carboxylate;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-pyrimidine-5-carboxylic acid;
ethyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)thiazole-4-carboxylate;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)thiazole-4-carboxylic acid;
methyl 6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-2,4-dimethylnicotinic acid;
3-(1-(4-chloro-3-fluorophenyl)-N,3,3-trimethyl-2,3-dihydro-1H-pyrrolo[3,2-b]pyridine-5-carboxamido)butanoic acid;
methyl 3-(1-(4-chloro-3-fluorophenyl)-N,3,3-trimethyl-2,3-dihydro-1H-pyrrolo[3,2-b]pyridine-5-carboxamido)butanoate;
methyl (R)-3-(1-(4-chloro-3-fluorophenyl)-N,3,3-trimethyl-2,3-dihydro-1H-pyrrolo[3,2-b]pyridine-5-carboxamido)butanoate;
(R)-3-(1-(4-chloro-3-fluorophenyl)-N,3,3-trimethyl-2,3-dihydro-1H-pyrrolo[3,2-b]pyridine-5-carboxamido)butanoic acid;
N-(1-carbamoylcyclopropyl)-1-(4-chloro-3-fluorophenyl)-N,3,3-trimethyl-2,3-dihydro-1H-pyrrolo[3,2-b]pyridine-5-carboxamide;
methyl 6-((2-(1-(4-chloro-3-fluorophenyl)-N,3,3-trimethyl-2,3-dihydro-1H-pyrrolo[3,2-b]pyridine-5-carboxamido)ethyl)(methyl)amino)-2,4-dimethylnicotinate;
6-((2-(1-(4-chloro-3-fluorophenyl)-N,3,3-trimethyl-2,3-dihydro-1H-pyrrolo[3,2-b]pyridine-5-carboxamido)ethyl)(methyl)amino)-2,4-dimethylnicotinic acid;
1-(4-chloro-3-fluorophenyl)-N-(1-hydroxy-2-methylpropan-2-yl)-3,3-dimethyl-2,3-dihydro-1H-pyrrolo[3,2-b]pyridine-5-carboxamide;
1-(4-chloro-3-fluorophenyl)-N-(2-hydroxy-2-methylpropyl)-3,3-dimethyl-2,3-dihydro-1H-pyrrolo[3,2-b]pyridine-5-carboxamide;
4-(1-(4-chloro-3-fluorophenyl)-3-ethyl-3-methyl-2,3-dihydro-1H-pyrrolo[3,2-b]pyridine-5-carbonyl)-3,3-dimethylpiperazin-2-one;
N-(1-carbamoylcyclopropyl)-1-(4-chloro-3-fluorophenyl)-3-ethyl-3-methyl-2,3-dihydro-1H-pyrrolo[3,2-b]pyridine-5-carboxamide;
4-(1′-(4-chloro-3-fluorophenyl)-1′,2,2′,3,5,6-hexahydrospiro[pyran-4,3′-pyrrolo[3,2-b]pyridine]-5′-carbonyl)-3,3-dimethylpiperazin-2-one;
6-(4-(1′-(4-chloro-3-fluorophenyl)-1′,2,2′,3,5,6-hexahydrospiro[pyran-4,3′-pyrrolo[3,2-b]pyridine]-5′-carbonyl)-3,3-dimethylpiperazin-1-yl)-2,4-dimethylnicotinic acid;
4-(1′-(4-chloro-3-fluorophenyl)-1′,2′-dihydrospiro[cyclopropane-1,3′-pyrrolo[3,2-b]pyridine]-5′-carbonyl)-3,3-dimethylpiperazin-2-one;
4-(1′-(4-chloro-3-fluorophenyl)-1′,2′,4,5-tetrahydro-2H-spiro[furan-3,3′-pyrrolo[3,2-b]pyridine]-5′-carbonyl)-3,3-dimethylpiperazin-2-one;
methyl 6-(4-(1′-(4-chloro-3-fluorophenyl)-1′,2′,4,5-tetrahydro-2H-spiro[furan-3,3′-pyrrolo[3,2-b]pyridine]-5′-carbonyl)-3,3-dimethylpiperazin-1-yl)-2,4-dimethylnicotinate;
6-(4-(1′-(4-chloro-3-fluorophenyl)-1′,2′,4,5-tetrahydro-2H-spiro[furan-3,3′-pyrrolo[3,2-b]pyridine]-5′-carbonyl)-3,3-dimethylpiperazin-1-yl)-2,4-dimethylnicotinic acid;
4-(8-(4-chloro-3-fluorophenyl)-3a-methyl-3,3a,8,8a-tetrahydro-2H-furo[3′,2′:4,5]pyrrolo[3,2-b]pyridine-5-carbonyl)-3,3-dimethylpiperazin-2-one;
cis-4-(8-(4-chloro-3-fluorophenyl)-3a-methyl-3,3a,8,8a-tetrahydro-2H-furo[3′,2′:4,5]pyrrolo[3,2-b]pyridine-5-carbonyl)-3,3-dimethylpiperazin-2-one;
4-((3aR,8aR)-8-(4-chloro-3-fluorophenyl)-3a-methyl-3,3a,8,8a-tetrahydro-2H-furo[3′,2′:4,5]pyrrolo[3,2-b]pyridine-5-carbonyl)-3,3-dimethylpiperazin-2-one;
4-((3aS,8aS)-8-(4-chloro-3-fluorophenyl)-3a-methyl-3,3a,8,8a-tetrahydro-2H-furo[3′,2′:4,5]pyrrolo[3,2-b]pyridine-5-carbonyl)-3,3-dimethylpiperazin-2-one;
methyl 2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)acetate;
2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)acetic acid;
ethyl 5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-1,3,4-thiadiazole-2-carboxylate;
(1-(4-chloro-3-fluorophenyl)-3,3-dimethyl-2,3-dihydro-1H-pyrrolo[3,2-b]pyridin-5-yl)(2,2-dimethyl-4-(1,3,4-thiadiazol-2-yl)piperazin-1-yl)methanone;
ethyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylthiazole-5-carboxylate;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylthiazole-5-carboxylic acid;
ethyl 2-(2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylthiazol-5-yl)acetate;
2-(2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylthiazol-5-yl)acetic acid;
methyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-5-methylthiazole-4-carboxylate;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-5-methylthiazole-4-carboxylic acid;
1-(1-(4-chloro-3-fluorophenyl)-3,3-dimethyl-2,3-dihydro-1H-pyrrolo[3,2-b]pyridine-5-carbonyl)-7,7-dimethyl-1,4-diazepan-5-one;
8-(1-(4-chloro-3-fluorophenyl)-3,3-dimethyl-2,3-dihydro-1H-pyrrolo[3,2-b]pyridine-5-carbonyl)-2,8-diazaspiro[4.5]decan-1-one;
8-(1-(4-chloro-3-fluorophenyl)-3,3-dimethyl-2,3-dihydro-1H-pyrrolo[3,2-b]pyridine-5-carbonyl)-2,8-diazaspiro[4.5]decan-3-one;
1-(1-(1-(4-chloro-3-fluorophenyl)-3,3-dimethyl-2,3-dihydro-1H-pyrrolo[3,2-b]pyridine-5-carbonyl)piperidin-4-yl)imidazolidin-2-one;
ethyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-(trifluoromethyl)thiazole-5-carboxylate;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-(trifluoromethyl)thiazole-5-carboxylic acid;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-N,N-dimethylthiazole-4-carboxamide;
(1-(4-chloro-3-fluorophenyl)-3,3-dimethyl-2,3-dihydro-1H-pyrrolo[3,2-b]pyridin-5-yl)(4-(4-(4-hydroxypiperidine-1-carbonyl)thiazol-2-yl)-2,2-dimethylpiperazin-1-yl)methanone;
(1-(4-chloro-3-fluorophenyl)-3,3-dimethyl-2,3-dihydro-1H-pyrrolo[3,2-b]pyridin-5-yl)(2,2-dimethyl-4-(4-(morpholine-4-carbonyl)thiazol-2-yl)piperazin-1-yl)methanone;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)thiazole-4-carboxamide;
methyl 6-(4-(1-(3-fluoro-4-methylphenyl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(3-fluoro-4-methylphenyl)-3,3-dimethyl-2,3-dihydro-1H-pyrrolo[3,2-b]pyridine-5-carbonyl)-3,3-dimethylpiperazin-1-yl)-2,4-dimethylnicotinic acid;
methyl 6-(4-(4-(4-chloro-3-fluorophenyl)-2,2-dimethyl-2,3,4,5-tetrahydropyrido[3,2-f][1,4]oxazepine-8-carbonyl)-3,3-dimethylpiperazin-1-yl)-2,4-dimethylnicotinate;
methyl 6-(4-(4-(4-chloro-3-fluorophenyl)-2,2-dimethyl-2,3,4,5-tetrahydropyrido[3,2-f][1,4]oxazepine-8-carbonyl)-3,3-dimethylpiperazin-1-yl)-2,4-dimethylnicotinate;
6-(4-(4-(4-chloro-3-fluorophenyl)-2,2-dimethyl-2,3,4,5-tetrahydropyrido[3,2-f][1,4]oxazepine-8-carbonyl)-3,3-dimethylpiperazin-1-yl)-2,4-dimethylnicotinic acid;
6-(4-(1′-(4-chloro-3-fluorophenyl)-3,3-difluoro-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
6-(4-(1′-(4-chloro-3-fluorophenyl)-3-fluoro-1′,2′-dihydrospiro[cyclobutane-1,3′-pyrrolo[3,2-b]pyridin]-2-en-5′-carbonyl)-3,3-dimethylpiperazin-1-yl)-2,4-dimethylnicotinic acid;
methyl 2-((3S,4S)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetate;
2-((3S,4S)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetic acid;
methyl 2-((3R,4R)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetate;
2-((3R,4R)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetic acid;
ethyl (2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)thiazole-4-carbonyl)glycinate;
(2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)thiazole-4-carbonyl)glycine;
methyl 2-((3S,4R)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetate;
2-((3S,4R)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetic acid;
methyl 6-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
(1-(4-chloro-3-fluorophenyl)-3,3-dimethyl-2,3-dihydro-1H-pyrrolo[3,2-b]pyridin-5-yl)(2,2-dimethyl-4-(4-(4-methylpiperazine-1-carbonyl)thiazol-2-yl)piperazin-1-yl)methanone;
methyl 6-(4-(1-(4-chloro-3-fluorophenyl)-3,3-diethyl-2,3-dihydro-1H-pyrrolo[3,2-b]pyridine-5-carbonyl)-3,3-dimethylpiperazin-1-yl)-2,4-dimethylnicotinate;
6-(4-(1-(4-chloro-3-fluorophenyl)-3,3-diethyl-2,3-dihydro-1H-pyrrolo[3,2-b]pyridine-5-carbonyl)-3,3-dimethylpiperazin-1-yl)-2,4-dimethylnicotinic acid;
methyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)isonicotinate;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)isonicotinic acid;
ethyl 3-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)isonicotinate;
3-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)isonicotinic acid;
ethyl 6-((1-(1-(4-chloro-3-fluorophenyl)-3,3-dimethyl-2,3-dihydro-1H-pyrrolo[3,2-b]pyridine-5-carbonyl)piperidin-3-yl)amino)nicotinate;
6-((1-(1-(4-chloro-3-fluorophenyl)-3,3-dimethyl-2,3-dihydro-1H-pyrrolo[3,2-b]pyridine-5-carbonyl)piperidin-3-yl)amino)nicotinic acid;
ethyl 6-((1-(1-(4-chloro-3-fluorophenyl)-3,3-dimethyl-2,3-dihydro-1H-pyrrolo[3,2-b]pyridine-5-carbonyl)piperidin-3-yl)(methyl)amino)nicotinate;
6-((1-(1-(4-chloro-3-fluorophenyl)-3,3-dimethyl-2,3-dihydro-1H-pyrrolo[3,2-b]pyridine-5-carbonyl)piperidin-3-yl)(methyl)amino)nicotinic acid hydrochloride;
methyl 6-(4-(1′-(3,4-difluorophenyl)-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
6-(4-(1′-(3,4-difluorophenyl)-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
methyl 2-(2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-4-yl)acetate;
2-(2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-4-yl)acetic acid;
ethyl 2-(5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-2-yl)acetate;
2-(5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-2-yl)acetic acid;
methyl 2-(6-(4-(1′-(4-chloro-3-fluorophenyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-(1′-(4-chloro-3-fluorophenyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
1′-(1-(4-chloro-3-fluorophenyl)-3,3-dimethyl-2,3-dihydro-1H-pyrrolo[3,2-b]pyridine-5-carbonyl)spiro[indoline-3,3′-pyrrolidin]-2-one;
8-(1-(4-chloro-3-fluorophenyl)-3,3-dimethyl-2,3-dihydro-1H-pyrrolo[3,2-b]pyridine-5-carbonyl)-1-oxa-3,8-diazaspiro[4.5]decan-2-one;
ethyl 6-(6-(1-(4-chloro-3-fluorophenyl)-3,3-dimethyl-2,3-dihydro-1H-pyrrolo[3,2-b]pyridine-5-carbonyl)-2,6-diazaspiro[3.3]heptan-2-yl)nicotinate;
6-(6-(1-(4-chloro-3-fluorophenyl)-3,3-dimethyl-2,3-dihydro-1H-pyrrolo[3,2-b]pyridine-5-carbonyl)-2,6-diazaspiro[3.3]heptan-2-yl)nicotinic acid;
methyl 2-(1-(1′-(4-chloro-3-fluorophenyl)-1′,2′-dihydrospiro[cyclobutane-1,3′-pyrrolo[3,2-b]pyridine]-5′-carbonyl)piperidin-4-yl)acetate;
2-(1-(1′-(4-chloro-3-fluorophenyl)-1′,2′-dihydrospiro[cyclobutane-1,3′-pyrrolo[3,2-b]pyridine]-5′-carbonyl)piperidin-4-yl)acetic acid;
4-(1′-(4-chloro-3-fluorophenyl)-1′,2′-dihydrospiro[cyclobutane-1,3′-pyrrolo[3,2-b]pyridine]-5′-carbonyl)-3,3-dimethylpiperazin-2-one;
methyl 2-(6-(4-(1′-(4-chloro-3-fluorophenyl)-3,3-difluoro-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-(1′-(4-chloro-3-fluorophenyl)-3,3-difluoro-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
ethyl 2-((1R,5S)-8-(1-(4-chloro-3-fluorophenyl)-3,3-dimethyl-2,3-dihydro-1H-pyrrolo[3,2-b]pyridine-5-carbonyl)-8-azabicyclo[3.2.1]octan-3-yl)acetate;
2-((1R,5S)-8-(1-(4-chloro-3-fluorophenyl)-3,3-dimethyl-2,3-dihydro-1H-pyrrolo[3,2-b]pyridine-5-carbonyl)-8-azabicyclo[3.2.1]octan-3-yl)acetic acid;
methyl 6-(4-(6-(5-(4-(5-(methoxycarbonyl)-4,6-dimethylpyridin-2-yl)-2,2-dimethylpiperazine-1-carbonyl)-3,3-dimethyl-2,3-dihydro-1H-pyrrolo[3,2-b]pyridin-1-yl)nicotinoyl)-3,3-dimethylpiperazin-1-yl)-2,4-dimethylnicotinate;
methyl 6-(4-(1-(5-chloropyridin-2-yl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(6-(5-(4-(5-carboxy-4,6-dimethylpyridin-2-yl)-2,2-dimethylpiperazine-1-carbonyl)-3,3-dimethyl-2,3-dihydro-1H-pyrrolo[3,2-b]pyridin-1-yl)nicotinoyl)-3,3-dimethylpiperazin-1-yl)-2,4-dimethylnicotinic acid;
6-(4-(1-(5-chloropyridin-2-yl)-3,3-dimethyl-2,3-dihydro-1H-pyrrolo[3,2-b]pyridine-5-carbonyl)-3,3-dimethylpiperazin-1-yl)-2,4-dimethylnicotinic acid;
methyl 6-(4-(3,3-dimethyl-1-(pyridin-2-yl)-2,3-dihydro-1H-pyrrolo[3,2-b]pyridine-5-carbonyl)-3,3-dimethylpiperazin-1-yl)-2,4-dimethylnicotinate;
ethyl 1-(1-(4-chloro-3-fluorophenyl)-3,3-dimethyl-2,3-dihydro-1H-pyrrolo[3,2-b]pyridine-5-carbonyl)-4-(pyridin-2-yl)piperidine-3-carboxylate;
(cis)-1-(1-(4-chloro-3-fluorophenyl)-3,3-dimethyl-2,3-dihydro-1H-pyrrolo[3,2-b]pyridine-5-carbonyl)-4-(pyridin-2-yl)piperidine-3-carboxylic acid;
(trans)-1-(1-(4-chloro-3-fluorophenyl)-3,3-dimethyl-2,3-dihydro-1H-pyrrolo[3,2-b]pyridine-5-carbonyl)-4-(pyridin-2-yl)piperidine-3-carboxylic acid;
methyl 6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-3-methylpicolinate;
6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-3-methylpicolinic acid;
methyl 5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-2-methoxynicotinate;
5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-2-methoxynicotinic acid;
methyl 6-(4-((1S,3R)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
methyl 6-(4-((1R,3R)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
methyl 6-(4-((3R)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
6-(4-((1S,3R)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
6-(4-((1R,3R)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
6-(4-((3R)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
methyl 6-(4-((1S,3S)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
methyl 6-(4-((1R,3S)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
methyl 6-(4-((3S)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
6-(4-((1S,3S)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
6-(4-((1R,3S)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
6-(4-((3S)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
methyl 2-(6-(4-(1-(4-chloro-3-fluorophenyl)-3,3-diethyl-2,3-dihydro-1H-pyrrolo[3,2-b]pyridine-5-carbonyl)-3,3-dimethylpiperazin-1-yl)pyridin-3-yl)acetate;
2-(6-(4-(1-(4-chloro-3-fluorophenyl)-3,3-diethyl-2,3-dihydro-1H-pyrrolo[3,2-b]pyridine-5-carbonyl)-3,3-dimethylpiperazin-1-yl)pyridin-3-yl)acetic acid;
methyl 5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)nicotinate;
methyl 5-(4-(1-(3-fluoro-4-(4-(5-(methoxycarbonyl)pyridin-3-yl)-2,2-dimethylpiperazine-1-carbonyl)phenyl)-3,3-dimethyl-2,3-dihydro-1H-pyrrolo[3,2-b]pyridine-5-carbonyl)-3,3-dimethylpiperazin-1-yl)nicotinate;
5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)nicotinic acid;
5-(4-(4-(5-(4-(5-carboxypyridin-3-yl)-2,2-dimethylpiperazine-1-carbonyl)-3,3-dimethyl-2,3-dihydro-1H-pyrrolo[3,2-b]pyridin-1-yl)-2-fluorobenzoyl)-3,3-dimethylpiperazin-1-yl)nicotinic acid;
methyl 2-(6-(4-((1s,3s)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-((1s,3s)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
methyl 6-(4-((1s,3s)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-((1s,3s)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 2-(6-(4-((1r,3r)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-((1r,3r)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
methyl 6-(4-((1r,3r)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-((1r,3r)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 2-(6-(4-(1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-(1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
methyl 6-(4-(1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-(1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)propanoate;
2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)propanoic acid;
methyl 6-(4-(1-(4-cyanocyclohexyl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(4-carbamoylcyclohexyl)-3,3-dimethyl-2,3-dihydro-1H-pyrrolo[3,2-b]pyridine-5-carbonyl)-3,3-dimethylpiperazin-1-yl)-2,4-dimethylnicotinic acid;
methyl 2-(6-(4-(1-(4,4-difluorocyclohexyl)-3,3-dimethyl-2,3-dihydro-1H-pyrrolo[3,2-b]pyridine-5-carbonyl)-3,3-dimethylpiperazin-1-yl)pyridin-3-yl)acetate;
2-(6-(4-(1-(4,4-difluorocyclohexyl)-3,3-dimethyl-2,3-dihydro-1H-pyrrolo[3,2-b]pyridine-5-carbonyl)-3,3-dimethylpiperazin-1-yl)pyridin-3-yl)acetic acid;
4-(5-(4-(4,6-dimethylpyridin-2-yl)-2,2-dimethylpiperazine-1-carbonyl)-3,3-dimethyl-2,3-dihydro-1H-pyrrolo[3,2-b]pyridin-1-yl)cyclohexane-1-carbonitrile;
methyl 2-(6-(4-(1-cyclopentyl-3,3-dimethyl-2,3-dihydro-1H-pyrrolo[3,2-b]pyridine-5-carbonyl)-3,3-dimethylpiperazin-1-yl)pyridin-3-yl)acetate;
2-(6-(4-(1-cyclopentyl-3,3-dimethyl-2,3-dihydro-1H-pyrrolo[3,2-b]pyridine-5-carbonyl)-3,3-dimethylpiperazin-1-yl)pyridin-3-yl)acetic acid;
methyl 2-(6-(4-(1-(4-cyanocyclohexyl)-3,3-dimethyl-2,3-dihydro-1H-pyrrolo[3,2-b]pyridine-5-carbonyl)-3,3-dimethylpiperazin-1-yl)pyridin-3-yl)acetate;
2-(6-(4-(1-(4-cyanocyclohexyl)-3,3-dimethyl-2,3-dihydro-1H-pyrrolo[3,2-b]pyridine-5-carbonyl)-3,3-dimethylpiperazin-1-yl)pyridin-3-yl)acetic acid;
methyl 2-(6-(4-(5-(3,4-difluorophenyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(3,4-difluorophenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 2-(6-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)-2-methylpropanoate;
2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)-2-methylpropanoic acid;
methyl 2-(6-(4-(5-(4-chloro-3-fluorophenyl)-8,8-dimethyl-5,6,7,8-tetrahydro-1,5-naphthyridine-2-carbonyl)-3,3-dimethylpiperazin-1-yl)pyridin-3-yl)acetate;
2-(6-(4-(5-(4-chloro-3-fluorophenyl)-8,8-dimethyl-5,6,7,8-tetrahydro-1,5-naphthyridine-2-carbonyl)-3,3-dimethylpiperazin-1-yl)pyridin-3-yl)acetic acid;
methyl 2-(6-(4-(4-(4-chloro-3-fluorophenyl)-7,7-dimethyl-4,5,6,7-tetrahydropyrazolo[1,5-a]pyrimidine-2-carbonyl)-3,3-dimethylpiperazin-1-yl)pyridin-3-yl)acetate;
2-(6-(4-(4-(4-chloro-3-fluorophenyl)-7,7-dimethyl-4,5,6,7-tetrahydropyrazolo[1,5-a]pyrimidine-2-carbonyl)-3,3-dimethylpiperazin-1-yl)pyridin-3-yl)acetic acid;
methyl 6-(4-(5-(3,4-difluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(3,4-difluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 6-(4-(5-cyclopentyl-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-cyclopentyl-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 6-(4-(5-(3-fluoro-4-methylphenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(3-fluoro-4-methylphenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 6-(4-(5-(4-chloro-3,5-difluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(4-chloro-3,5-difluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 6-(4-(5-(3-chloro-4-methylphenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(3-chloro-4-methylphenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 6-(4-(5-(3-chloro-4-fluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(3-chloro-4-fluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 6-(4-(5-(3-chloro-4-(trifluoromethyl)phenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(3-chloro-4-(trifluoromethyl)phenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 2-(6-(4-(5-(4-chloro-3-fluorophenyl)-7,7-dimethyl-4,5,6,7-tetrahydropyrazolo[1,5-a]pyrazine-2-carbonyl)-3,3-dimethylpiperazin-1-yl)pyridin-3-yl)acetate;
2-(6-(4-(5-(4-chloro-3-fluorophenyl)-7,7-dimethyl-4,5,6,7-tetrahydropyrazolo[1,5-a]pyrazine-2-carbonyl)-3,3-dimethylpiperazin-1-yl)pyridin-3-yl)acetic acid;
methyl 2-(6-(4-(3-(4-chloro-3-fluorobenzyl)-1-isobutyl-3-methyl-2,3-dihydro-1H-pyrrolo[2,3-b]pyridine-6-carbonyl)-3,3-dimethylpiperazin-1-yl)pyridin-3-yl)acetate;
2-(6-(4-(3-(4-chloro-3-fluorobenzyl)-1-isobutyl-3-methyl-2,3-dihydro-1H-pyrrolo[2,3-b]pyridine-6-carbonyl)-3,3-dimethylpiperazin-1-yl)pyridin-3-yl)acetic acid;
methyl 6-(4-((1s,3s)-1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-((1s,3s)-1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 6-(4-((1r,3r)-1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-((1r,3r)-1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 6-(4-(1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-(1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 2-(6-(4-(5-(3-fluoro-4-methylphenyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(3-fluoro-4-methylphenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 2-(6-(4-(5-(3-chloro-4-fluorophenyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(3-chloro-4-fluorophenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 6-(4-(5′-(4-chloro-3-fluorophenyl)-5′,6′-dihydrospiro[cyclopentane-1,7′-pyrrolo[2,3-b]pyrazine]-2′-carbonyl)-3,3-dimethylpiperazin-1-yl)-2,4-dimethylnicotinate;
6-(4-(5′-(4-chloro-3-fluorophenyl)-5′,6′-dihydrospiro[cyclopentane-1,7′-pyrrolo[2,3-b]pyrazine]-2′-carbonyl)-3,3-dimethylpiperazin-1-yl)-2,4-dimethylnicotinic acid;
methyl 6-(4-(1-acetyl-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(1,3-dihydroisobenzofuran-5-yl)-3,3-dimethyl-2,3-dihydro-1H-pyrrolo[3,2-b]pyridine-5-carbonyl)-3,3-dimethylpiperazin-1-yl)-2,4-dimethylnicotinic acid;
methyl 2-(6-(4-(5-(4-fluorophenethyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(4-fluorophenethyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 6-(4-(5-(4-fluorophenethyl)-7,7-dimethyl-6,7-dihydro-5H-pyrrolo[2,3-b]pyrazine-2-carbonyl)-3,3-dimethylpiperazin-1-yl)-2,4-dimethylnicotinate;
2-(6-(4-(5-(4-fluorophenethyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 6-(4-(1′-(4-chloro-3-fluorophenyl)-3,3-dimethyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-(1′-(4-chloro-3-fluorophenyl)-3,3-dimethyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 2-(6-(4-(7,7-dimethyl-5-(3,4,5-trifluorophenyl)-6,7-dihydro-5H-pyrrolo[2,3-b]pyrazine-2-carbonyl)-3,3-dimethylpiperazin-1-yl)pyridin-3-yl)acetate;
2-(6-(4-(7,7-dimethyl-5-(3,4,5-trifluorophenyl)-6,7-dihydro-5H-pyrrolo[2,3-b]pyrazine-2-carbonyl)-3,3-dimethylpiperazin-1-yl)pyridin-3-yl)acetic acid;
methyl 2-(6-(4-(5-(3-chloro-4-(trifluoromethyl)phenyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(3-chloro-4-(trifluoromethyl)phenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 2-(6-(4-(5-(3,4-dichlorophenyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(3,4-dichlorophenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 6-(4-(5′-(3,4-difluorophenyl)-5′,6′-dihydrospiro[cyclopentane-1,7′-pyrrolo[2,3-b]pyrazine]-2′-carbonyl)-3,3-dimethylpiperazin-1-yl)-2,4-dimethylnicotinate;
6-(4-(5′-(3,4-difluorophenyl)-5′,6′-dihydrospiro[cyclopentane-1,7′-pyrrolo[2,3-b]pyrazine]-2′-carbonyl)-3,3-dimethylpiperazin-1-yl)-2,4-dimethylnicotinic acid;
methyl 6-(4-((1r,3r)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-((1r,3r)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 6-(4-((1s,3s)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-((1s,3s)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 6-(4-(1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-(1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 2-(6-(4-(5′-(3,4-difluorophenyl)-5′,6′-dihydrospiro[cyclopentane-1,7′-pyrrolo[2,3-b]pyrazine]-2′-carbonyl)-3,3-dimethylpiperazin-1-yl)pyridin-3-yl)acetate;
2-(6-(4-(5′-(3,4-difluorophenyl)-5′,6′-dihydrospiro[cyclopentane-1,7′-pyrrolo[2,3-b]pyrazine]-2′-carbonyl)-3,3-dimethylpiperazin-1-yl)pyridin-3-yl)acetic acid;
methyl 2-(6-(4-(5′-(4-chloro-3-fluorophenyl)-5′,6′-dihydrospiro[cyclobutane-1,7′-pyrrolo[2,3-b]pyrazine]-2′-carbonyl)-3,3-dimethylpiperazin-1-yl)pyridin-3-yl)acetate;
2-(6-(4-(5′-(4-chloro-3-fluorophenyl)-5′,6′-dihydrospiro[cyclobutane-1,7′-pyrrolo[2,3-b]pyrazine]-2′-carbonyl)-3,3-dimethylpiperazin-1-yl)pyridin-3-yl)acetic acid;
methyl 6-(4-(5′-(3,4-difluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-2,4-dimethylnicotinate;
6-(4-(5′-(3,4-difluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-2,4-dimethylnicotinic acid;
methyl 6-(4-(3,3-dimethyl-5′-(3,4,5-trifluorophenyl)-5′,6′-dihydrospiro[cyclobutane-1,7′-pyrrolo[2,3-b]pyrazine]-2′-carbonyl)-3,3-dimethylpiperazin-1-yl)-2,4-dimethylnicotinate;
6-(4-(3,3-dimethyl-5′-(3,4,5-trifluorophenyl)-5′,6′-dihydrospiro[cyclobutane-1,7′-pyrrolo[2,3-b]pyrazine]-2′-carbonyl)-3,3-dimethylpiperazin-1-yl)-2,4-dimethylnicotinic acid;
methyl 6-(4-(5′-(4-chloro-3-fluorophenyl)-5′,6′-dihydrospiro[cyclobutane-1,7′-pyrrolo[2,3-b]pyrazine]-2′-carbonyl)-3,3-dimethylpiperazin-1-yl)-2,4-dimethylnicotinate;
6-(4-(5′-(4-chloro-3-fluorophenyl)-5′,6′-dihydrospiro[cyclobutane-1,7′-pyrrolo[2,3-b]pyrazine]-2′-carbonyl)-3,3-dimethylpiperazin-1-yl)-2,4-dimethylnicotinic acid;
methyl 2-(6-(4-((1s,3s)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-((1s,3s)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
methyl 2-(6-(4-((1r,3r)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-((1r,3r)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
methyl 2-(6-(4-(1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-(1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
methyl 2-(6-(4-(5-(4-chloro-3,5-difluorophenyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(4-chloro-3,5-difluorophenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 2-(6-(4-(5′-(4-chloro-3-fluorophenyl)-5′,6′-dihydrospiro[cyclopentane-1,7′-pyrrolo[2,3-b]pyrazine]-2′-carbonyl)-3,3-dimethylpiperazin-1-yl)pyridin-3-yl)acetate;
2-(6-(4-(5′-(4-chloro-3-fluorophenyl)-5′,6′-dihydrospiro[cyclopentane-1,7′-pyrrolo[2,3-b]pyrazine]-2′-carbonyl)-3,3-dimethylpiperazin-1-yl)pyridin-3-yl)acetic acid;
methyl 2-(6-(4-(5′-(3,4-difluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)pyridin-3-yl)acetate;
2-(6-(4-(5′-(3,4-difluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)pyridin-3-yl)acetic acid;
methyl 6-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-2,4-dimethylnicotinate;
6-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-2,4-dimethylnicotinic acid;
methyl 6-(4-(5-(3,4-dichlorophenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(3,4-dichlorophenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 2-(6-(4-(5-(3-chloro-4-methylphenyl)-7,7-dimethyl-6,7-dihydro-5H-pyrrolo[2,3-b]pyrazine-2-carbonyl)-3,3-dimethylpiperazin-1-yl)pyridin-3-yl)acetate
2-(6-(4-(5-(3-chloro-4-methylphenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 2-(6-(4-(5-(4-chloro-3-fluorophenyl)-8-methoxy-7,7-dimethyl-5,6,7,8-tetrahydro-1,5-naphthyridine-2-carbonyl)-3,3-dimethylpiperazin-1-yl)pyridin-3-yl)acetate;
2-(6-(4-(5-(4-chloro-3-fluorophenyl)-8-methoxy-7,7-dimethyl-5,6,7,8-tetrahydro-1,5-naphthyridine-2-carbonyl)-3,3-dimethylpiperazin-1-yl)pyridin-3-yl)acetic acid;
methyl 2-(6-(4-(1-(4-chloro-3-fluorophenyl)-6-methoxy-3,3-dimethyl-2,3-dihydro-1H-pyrrolo[3,2-b]pyridine-5-carbonyl)-3,3-dimethylpiperazin-1-yl)pyridin-3-yl)acetate;
2-(6-(4-(1-(4-chloro-3-fluorophenyl)-6-methoxy-3,3-dimethyl-2,3-dihydro-1H-pyrrolo[3,2-b]pyridine-5-carbonyl)-3,3-dimethylpiperazin-1-yl)pyridin-3-yl)acetic acid;
methyl 2-(6-(4-(3-(4-chloro-3-fluorophenyl)-1-isobutyl-3-methyl-2,3-dihydro-1H-pyrrolo[2,3-b]pyridine-6-carbonyl)-3,3-dimethylpiperazin-1-yl)pyridin-3-yl)acetate;
2-(6-(4-(3-(4-chloro-3-fluorophenyl)-1-isobutyl-3-methyl-2,3-dihydro-1H-pyrrolo[2,3-b]pyridine-6-carbonyl)-3,3-dimethylpiperazin-1-yl)pyridin-3-yl)acetic acid;
2-(6-(4-(1-(4-chloro-3-fluorophenyl)-6-hydroxy-3,3-dimethyl-2,3-dihydro-1H-pyrrolo[3,2-b]pyridine-5-carbonyl)-3,3-dimethylpiperazin-1-yl)pyridin-3-yl)acetic acid;
methyl 8-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-8-oxooctanoate;
8-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-8-oxooctanoic acid;
N-(37-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-3-methyl-4,17,30,37-tetraoxo-7,10,13,20,23,26-hexaoxa-3,16,29-triazaheptatriacontyl)-N-methylpalmitamide;
methyl 4-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)-2,6-dimethylnicotinate;
4-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)-2,6-dimethylnicotinic acid;
methyl 2-(6-(4-(1-(4-chloro-3-fluorophenyl)-3,3-dimethyl-2,3-dihydro-1H-pyrido[2,3-b][1,4]oxazine-6-carbonyl)-3,3-dimethylpiperazin-1-yl)pyridin-3-yl)acetate;
2-(6-(4-(1-(4-chloro-3-fluorophenyl)-3,3-dimethyl-2,3-dihydro-1H-pyrido[2,3-b][1,4]oxazine-6-carbonyl)-3,3-dimethylpiperazin-1-yl)pyridin-3-yl)acetic acid;
1-(1-((5′-(4-chloro-3-fluorophenyl)-3,3-dimethyl-5′,6′-dihydrospiro[cyclobutane-1,7′-pyrrolo[2,3-b]pyrazin]-2′-yl)sulfonyl)piperidin-4-yl)imidazolidin-2-one;
methyl 2-(6-(4-(4-(4-chloro-3-fluorophenyl)-2,2-dimethyl-3,4-dihydro-2H-pyrazino[2,3-b][1, 4]oxazine-7-carbonyl)-3,3-dimethylpiperazin-1-yl)pyridin-3-yl)acetate;
2-(6-(4-(4-(4-chloro-3-fluorophenyl)-2,2-dimethyl-3,4-dihydro-2H-pyrazino[2,3-b][1,4]oxazine-7-carbonyl)-3,3-dimethylpiperazin-1-yl)pyridin-3-yl)acetic acid;
methyl 2-(6-(4-(5-(4-chloro-3-fluorophenyl)-7,7-dimethyl-4,5,6,7-tetrahydrothiazolo[5,4-c]pyridine-2-carbonyl)-3,3-dimethylpiperazin-1-yl)pyridin-3-yl)acetate;
2-(6-(4-(5-(4-chloro-3-fluorophenyl)-7,7-dimethyl-4,5,6,7-tetrahydrothiazolo[5,4-c]pyridine-2-carbonyl)-3,3-dimethylpiperazin-1-yl)pyridin-3-yl)acetic acid;
tert-butyl 2-(4-(5-(2-methoxy-2-oxoethyl)pyridin-2-yl)-2,2-dimethylpiperazine-1-carbonyl)-7,7-dimethyl-2,4,6,7-tetrahydro-5H-pyrazolo[4,3-c]pyridine-5-carboxylate;
methyl 2-(6-(4-(5-(4-chloro-3-fluorophenyl)-7,7-dimethyl-4,5,6,7-tetrahydro-2H-pyrazolo[4,3-c]pyridine-2-carbonyl)-3,3-dimethylpiperazin-1-yl)pyridin-3-yl)acetate;
2-(6-(4-(5-(4-chloro-3-fluorophenyl)-7,7-dimethyl-4,5,6,7-tetrahydro-2H-pyrazolo[4,3-c]pyridine-2-carbonyl)-3,3-dimethylpiperazin-1-yl)pyridin-3-yl)acetic acid;
methyl 6-(4-(1-(3-fluoro-4-(trifluoromethyl)phenyl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(3-fluoro-4-(trifluoromethyl)phenyl)-3,3-dimethyl-2,3-dihydro-1H-pyrrolo[3,2-b]pyridine-5-carbonyl)-3,3-dimethylpiperazin-1-yl)-2,4-dimethylnicotinic acid;
methyl 6-(4-(7,7-dimethyl-5-(3,4,5-trifluorophenyl)-6,7-dihydro-5H-pyrrolo[2,3-b]pyrazine-2-carbonyl)-3,3-dimethylpiperazin-1-yl)-2,4-dimethylnicotinate;
6-(4-(7,7-dimethyl-5-(3,4,5-trifluorophenyl)-6,7-dihydro-5H-pyrrolo[2,3-b]pyrazine-2-carbonyl)-3,3-dimethylpiperazin-1-yl)-2,4-dimethylnicotinic acid;
methyl 6-(4-(1-(4-cyclopropylphenyl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(4-cyclopropylphenyl)-3,3-dimethyl-2,3-dihydro-1H-pyrrolo[3,2-b]pyridine-5-carbonyl)-3,3-dimethylpiperazin-1-yl)-2,4-dimethylnicotinic acid;
methyl 2-(6-(4-(5-(4-chloro-3-fluorophenyl)-3,7,7-trimethyl-4,5,6,7-tetrahydro-3H-imidazo[4,5-c]pyridine-2-carbonyl)-3,3-dimethylpiperazin-1-yl)pyridin-3-yl)acetate;
2-(6-(4-(5-(4-chloro-3-fluorophenyl)-3,7,7-trimethyl-4,5,6,7-tetrahydro-3H-imidazo[4,5-c]pyridine-2-carbonyl)-3,3-dimethylpiperazin-1-yl)pyridin-3-yl)acetic acid;
4-(5′-(3,4-difluorophenyl)-3,3-dimethyl-5′,6′-dihydrospiro[cyclobutane-1,7′-pyrrolo[2,3-b]pyrazine]-2′-carbonyl)-3,3-dimethylpiperazin-2-one;
or a pharmaceutically acceptable salt or solvate of any one of the above-mentioned compounds.

The present invention also relates to each of the intermediates described further below in the examples section of this specification, including any one of these intermediates in non-salt form and/or non-solvated form, or in the form of a salt or solvate (e.g., a pharmaceutically acceptable salt or solvate) of the respective compound. Such intermediates can be used, in particular, in the synthesis of the compounds of formula (I).

For a person skilled in the field of synthetic chemistry, various ways for the preparation of the compounds of general formula (I) and their pharmaceutically acceptable salts and solvates will be readily apparent. For example, the compounds of the invention can be prepared in accordance with, or in analogy to, the synthetic routes described in detail in the examples section. In particular, the compounds of formula (I) can be synthesized in accordance with the methods described in the following general schemes (general disconnections).

### Schematic Overview:

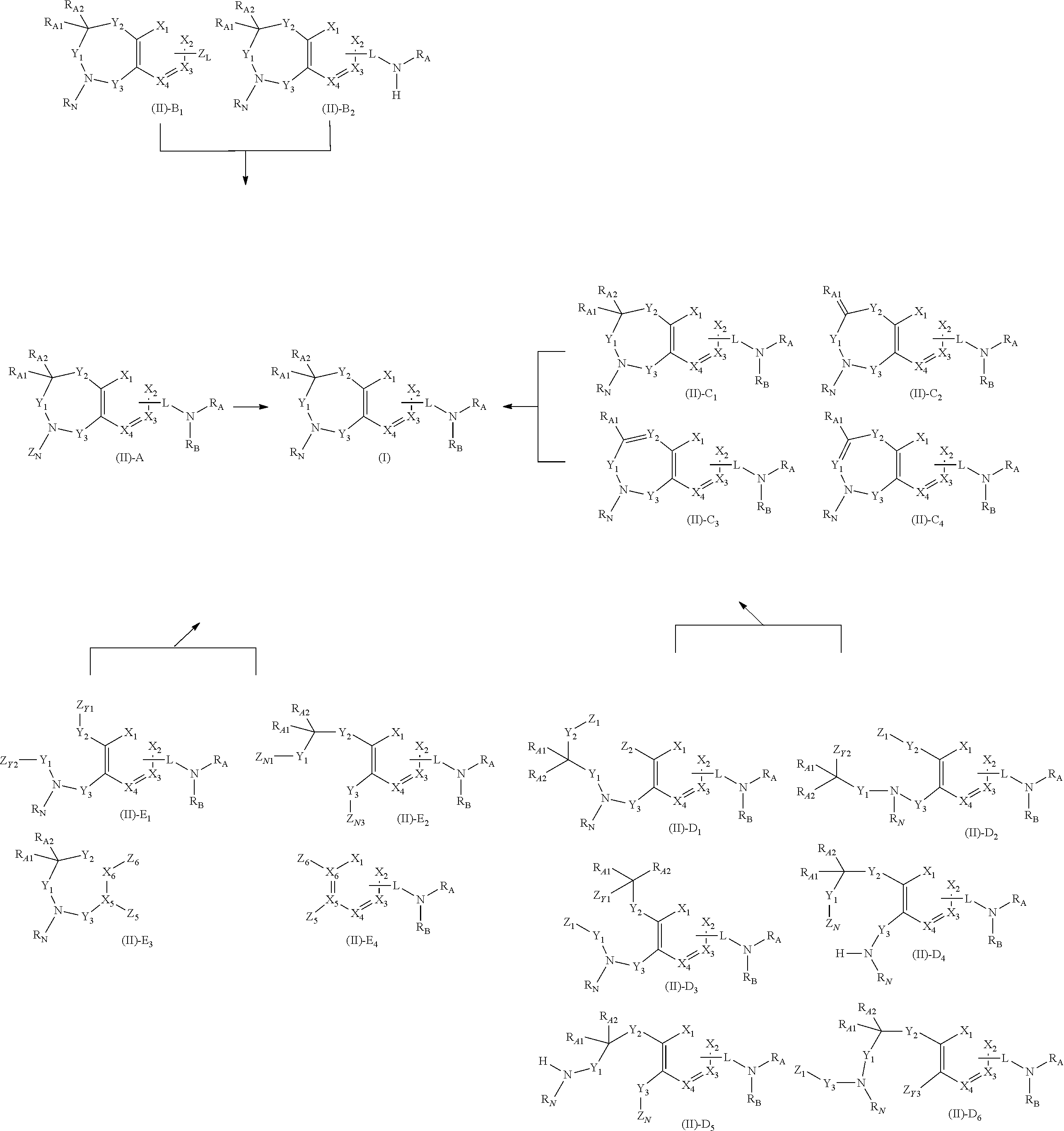

Compound of formula (I) can be obtained from a precursor (II)-A according to the general disconnection A:

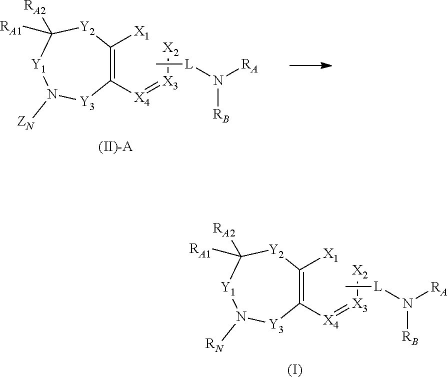

With Z_{N} being a hydrogen atom:
By a N-derivation known to the person skilled in the art, with the appropriate partner (based on the works described in Chem. Rev., 2016, 116, 12564-12649, Chem. Rev. 2019, 119, 11857-11911, Chem. Rev. 2019, 119, 12491-12523, Chem. Rev. 2019, 119, 11245-11290, Chem. Soc. Rev., 2014, 3525-3550, or Nature, 2020, 581, 415-420)
By aniline synthesis from the appropriate aldehyde or keto derivative (based on the works described in Org. Lett., 2012, 14, 5606-5609 or Nature, 2020, 584, 75-81).

With Z_{N} being a methyl group:
By N-arylation (based on the work described in Org. Lett., 2013, 15, 5452-5455) with the appropriate aryne precursor.

With Z_{N} being a benzoic group:
By metal catalyzed debenzoylative N-arylation with the appropriate organometallic partner (based on the work described in J. Org. Chem., 2006, 71, 219-224)

With Z_{N} being a substituted carbonyl group:
By rearrangement of the corresponding amide derivative (based on the work described in Synlett 2013, 24, 1448-1454), or by rearrangement of the corresponding carbamate derivative (based on the work described in J. Am. Chem. Soc., 2019, 141, 7262-7265)
By metal catalyzed deacylative N-arylation with the appropriate halogenated or pseudo-halogenated aryl (based on the works described in Org. Lett. 2021, 23, 3, 687-691 or J. Org. Chem., 2012, 77, 9236-9239)

Compounds of formula (I) can be obtained from a precursor (II)-B₁ according to the general disconnection B₁:

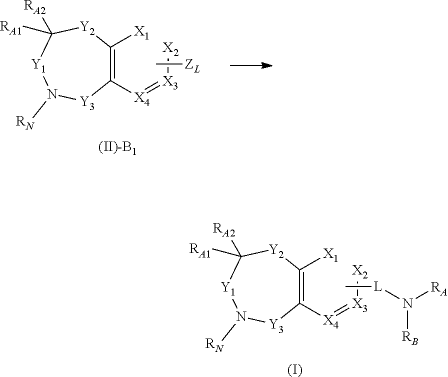

With Z_{L} being a hydrogen atom (based on the work described in ARKIVOC 2013, 1, 154-174, ARKIVOC 2001, 1, 242-268 or Synthesis 2011, 20, 3209-3219):
By N-oxidation followed by a sequence leading to the carboxylic, sulfonic or sulfinic acid and subsequent amide, sulfonamide or sulfinamide synthesis with the appropriate amine.
By N-oxidation followed by a sequence leading to the conversion of Z_{L} into a halogen or a pseudo-halogen, followed by a transformation or a sequence described below.
By carbon-hydrogen bond activation with a metal leading to the conversion of Z_{L} into a halogen, a pseudo-halogen, or an organometallic group, followed by a transformation or a sequence described below.

With Z_{L} being a hydroxy group (based on the work described in Org. Process Res. Dev. 2004, 8, 62-71 or Tet. Lett., 1992, 33, 1181-1184):
By a transformation or sequence known to the person skilled in the art of Z_{L} into a halogen or pseudo-halogen, followed by a transformation or a sequence described below.

With Z_{L} being a halogen or a pseudo-halogen, or an organometallic group:
By a direct, metal catalyzed amino-carbonylation reaction, sulfinamide or sulfonamide synthesis with the appropriate amine (based on the work described in RSC Adv., 2014, 4, 10367-10389 or Synthesis, 2008, 311-312)
By a transformation or a sequence known to the person skilled in the art leading to a carboxylic, sulfonic or sulfinic acid, followed by amide, sulfonamide or sulfinimide synthesis with the appropriate amine (based on the work described in J. Org. Chem., 2008, 73, 3967-3969)

Compounds of formula (I) can be obtained from a precursor (II)-B₂ according to the general disconnection B₂:

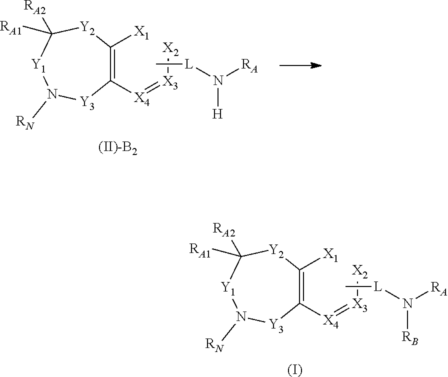

By a transformation or a sequence known to the person skilled in the art leading to a tertiary amide, sulfonamide or sulfinimide by for example a sequence including deprotonation and N-substitution with the appropriate partner, or a metal catalyzed N-derivation.

Compounds of formula (I) can be obtained from a precursor (II)-C₁ according to the general disconnection C₁:

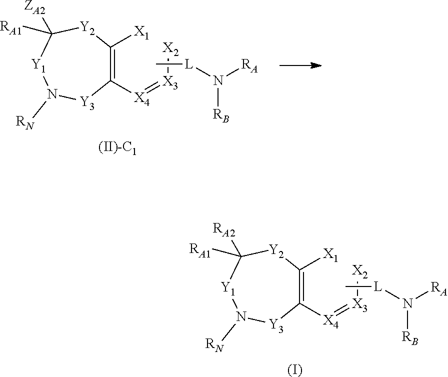

With Z_{A2} being a halogen or a pseudo-halogen and R_{A1}, Y₁ or Y₂ linked with a carbonyl (based on the works described in ChemistryOpen, 2020, 9, 100-17, or Acc. Chem. Res. 2008, 41, 11, 1545-1554):
By halogen metal exchange, followed by derivation by metal catalysed coupling with the appropriate partner.
By halogen metal exchange, followed by nucleophilic displacement or nucleophilic addition on the appropriate partner.
By direct nucleophilic displacement of the halogen or pseudo-halogen with the appropriate nucleophile.

With Z_{A2} being a hydrogen atom and R_{A1}, Y₁ or Y₂ linked with a carbonyl (based on the works described in ChemistryOpen, 2020, 9, 100-17, or Acc. Chem. Res. 2008, 41, 11, 1545-1554):
By metalation followed by derivation by metal catalysed coupling with the appropriate partner.
By metalation followed by nucleophilic displacement or nucleophilic addition on the appropriate partner.
By direct nucleophilic displacement of the halogen or pseudo-halogen with the appropriate nucleophile.
By oxidative coupling (based on the work described in Angew. Chem. Int. Ed. 2017, 56, 5921-5925)

With Z_{A2} being a hydrogen atom and Y₁ being a bond:
By photoredox catalysed alpha derivation of amine with the appropriate radical acceptor (based on the works described in J. Am. Chem. Soc. 2020, 15, 142, 11972-11977, or Chem. Rev., 2013, 113, 5322-5363).

With Z_{A2} being a halogen or a pseudo-halogen and Y₂ being a bond:
By metal catalysed coupling with the appropriate partner (based on the works described in Org. Lett., 2009, 11, 23, 5514-5517)

Compounds of formula (I) can be obtained from a precursor (II)—C₂, (II)-C₃ or (II)—C₄ according to general disconnection C_{2/3/4}:

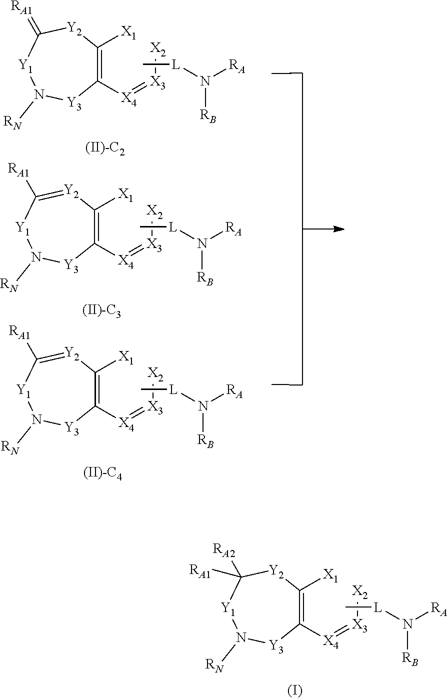

By [2+1]cycloaddition with the appropriate partner (based on the works described in Chem. Rev. 2017, 117, 18, 11651-11679).
By epoxidation followed by epoxide opening with the appropriate partner (based on the works described in Chem. Soc. Rev., 2011, 40, 1722-1760, J. Am. Chem. Soc. 1993, 115, 19, 8867-8868, or Synlett, 2005, 8, 1199-1222).
By [2+2]cycloaddition with the appropriate partner (based on the works described in Eur. J. Org. Chem., 2020, 10, 1310-1326, or Chem. Rev. 2016, 116, 9748-9815).
By [2+2]cycloaddition with the appropriate carbonyle, eventually followed with the corresponding oxetane opening with the appropriate partner (based on the works described in Molecules, 2013, 18, 11384-11428, or Chem. Rev., 2016, 116, 12150-12233).
By [2+3]cycloaddition with the appropriate partner (based on the works described in Beilstein J. Org. Chem. 2020, 16, 3015-3031, or Chem. Rev. 2005, 105, 2765-2809).
By [2+4] Diels-Alder cycloaddition with the appropriate diene or with the appropriate dienophile (based on the works described in Chem. Rev. 1942, 31, 2, 319-523, or Chem. Soc. Rev., 2018, 47, 7926).
By a nucleophilic addition known to the person skilled in the art with (II)-C₃ containing a conjugated double bond (Y₂ being —CZ—) or an imine (Y₂ being a N) or with (II)-C₄ containing an α,β-unsaturated carbonyl (Y₁ being —CZ₂—C(O)—) sulfononyl (Y₁ being —CZ₂—S(O)₂—) or an imine (Y₁ being a bond).
By an electrophilic addition known to the person skilled in the art on the exo cyclic double bond on (II)-C₄(Y₁ being —CZ—).

Additionally, specific intermediates (II)-C₂ (II)-C₃ and (II)-C₄ can be obtained, or can be precursors to obtain compounds of formula (I), using synthetic strategies residing on the dearomatization of respective aromatic compounds (based on the works described in Synlett, 2020, 31, 1775-1788, Org. Chem. Front., 2020, 7, 3967-3998, Tetrahedron, 2015, 71, 3549-359, Org. Lett. 2019, 21, 4459-4463, Synthesis, 2022, 54, 92-110, Chem Sci. 2017, 8, 7112-7118, Org. Biomol. Chem., 2014, 12, 4807-4815, or Tetrahedron, 2019, 75, 2063-2097).

Compounds of formula (I) can be obtained from a precursor (II)-D₁ (II)-D₂, (II)-D₃, or (II)-D₆ according to general disconnection D_{1/2/3/6}:

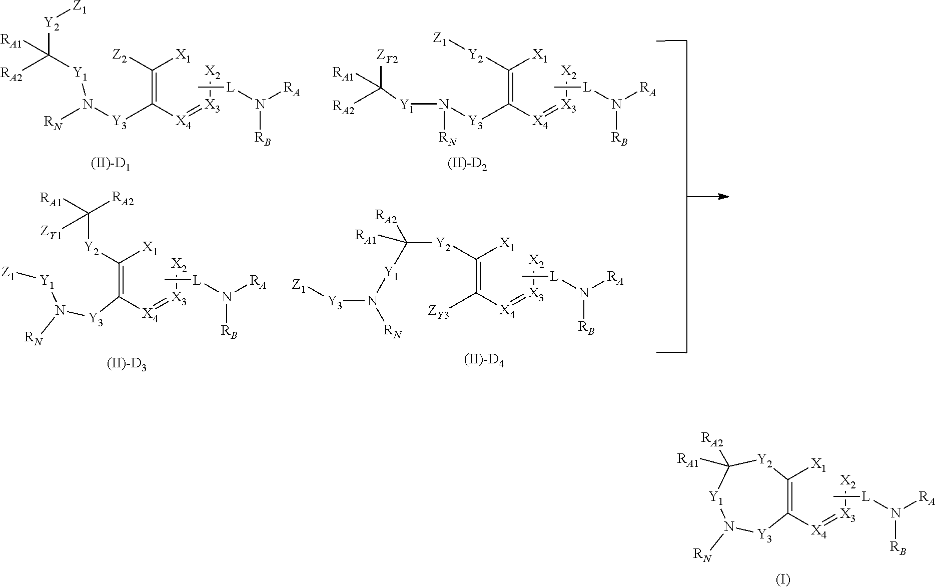

With Z₁ being a hydrogen or an organometallic group, or a halogen or pseudo halogen and Z₂, Z_{Y1}, Z_{Y2}, Z_{Y3} being respectively a halogen or a pseudo halogen, or a hydrogen or an organometallic group:
By a metal catalyzed coupling reaction known to the person skilled in the art.

With Z₁ being a hydrogen, or a halogen or pseudo halogen and Z₂ Z_{Y1}, Z_{Y2}, Z_{Y3}, being respectively a halogen or a pseudo halogen, or a hydrogen:
By a guided metalation followed by an intramolecular nucleophilic substitution or nucleophilic aromatic substitution known to the person skilled in the art.
By a nucleophilic substitution or nucleophilic aromatic substitution known to the person skilled in the art.

With Z₁ and Z₂, Z_{Y1}, Z_{Y2}, Z_{Y3} being both halogens or pseudo halogens:
By a metal catalyzed reductive coupling (based on the works described in Chem. Eur. J. 2014, 20, 15334-15338, or Chem. Eur. J. 2014, 20, 6828-6842).

With Z₁ and Z₂, Z_{Y1}, Z_{Y2}, Z_{Y3} being the appropriate partners:
By a suitable cycloaddition mentioned in general disconnection C_{2/3/4}

Compounds of formula (I) can be obtained from a precursor (II)-D₄ or (II)-D₅ according to general disconnection D_{4/5}:

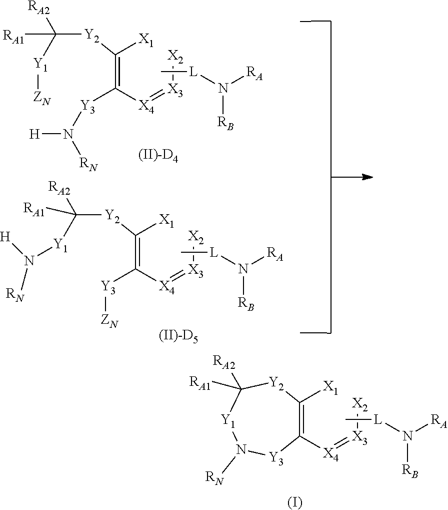

With Z_{N} being a carbonyl:
By a reductive amination reaction or amide synthesis known to the person skilled in the art.
By a Petasis reaction (based on the works described in Chem. Rev. 2019, 119, 11245-11290, or RSC Adv., 2015, 5, 76337-76341).

With Z_{N} being a halogen or pseudo-halogen, or an organometallic group:
By a nucleophilic substitution known to the person skilled in the art.
By a metal catalyzed coupling reaction (based on the works described in Chem. Rev. 2016, 116, 19, 12564-12649, Chem. Soc. Rev., 2014, 43, 3525-3550 or Chem. Rev. 2019, 119, 24, 12491-12523).

Compounds of formula (I) can be obtained from a precursor (II)-E₁ according to general disconnection E₁:

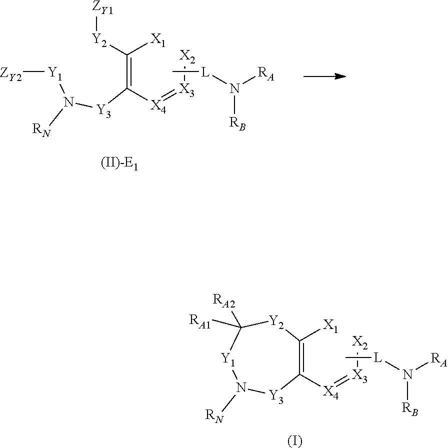

By a cross-coupling reaction, reductive coupling or cross metathesis known to the person skilled in the art.
By a Petasis or Mannich reaction (based respectively on the works described in Chem. Rev. 2019, 119, 11245-11290, RSC Adv., 2015, 5, 76337-76341, Org. Chem. Front., 2018, 5, 1049-1066, or Synthesis 2013, 45, 2769-2812).

Compounds of formula (I) can be obtained from a precursor (II)-E₂ according to general disconnection E₂:

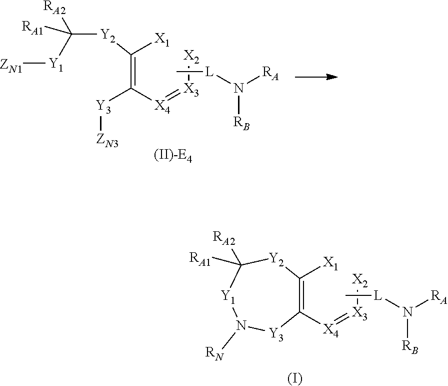

By double reductive amination with R_{N}—NH₂
By double nucleophilic (aromatic) substitution with R_{N}—NH₂
By one pot reductive amination and nucleophilic substitution with R_{N}—NH₂

Compounds of formula (I) can be obtained from precursor (II)-E3 and/or (II)-E₄ according to general disconnection E_{3/4}:

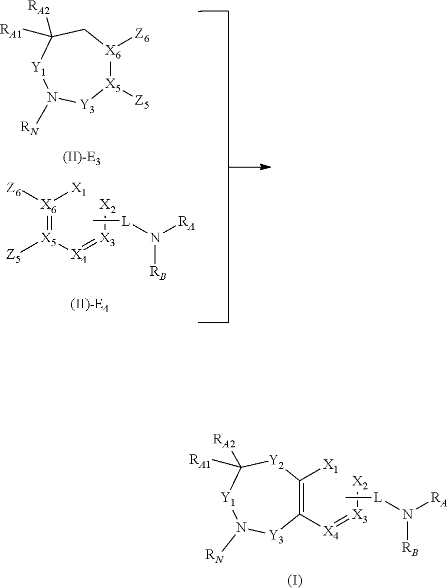

By a heterocycle synthesis from precursor (II)-E3 known to the person skilled in the art, and more specifically by specific methods described in Topics in Current Chemistry, 2019, 377, 21, Synthetic Communications, 2020, 50, 1251-1285, Org. Chem. Front., 2019, 6, 2120-2141, Chem. Rev. 2011, 111, 4, 2937-2980, or Synlett, 2011, 2387-2391.
By cycloaddition reactions leading to a heteroaromatic cycle synthesis known to the person skilled in the art, and more specifically sequences based on the works described in Org. Chem. Front., 2014, 1, 1010-1015, J. Am. Chem. Soc. 1999, 121, 54-62, Acc. Chem. Res. 2020, 53, 4, 773-781 or C. R. Chimie, 2017, 20, 643-647.
By cycloaddition leading to a non-aromatic heterocycle synthesis known to the person skilled in the art, and more specifically hetero-aryne transformations based on the works described in Angew. Chem. Int. Ed., 2015, 54, 11765-11769, or Chem. Rev. 2021, 121, 3892-4044.

Using and combining the above-mentioned general disconnections A, B₁, B₂, C₁, C_{2/3/4}, D_{1/2/3/6}, D_{4/5}, E₁, E₂, E_{3/4}, and available literature enables the synthesis of any compounds of formula (I) from commercially available compounds or from compounds whose synthesis is already described in the literature or compounds which are accessible with a method known in the art.

The following definitions apply throughout the present specification and the claims, unless specifically indicated otherwise.

The term “hydrocarbon group” refers to a group consisting of carbon atoms and hydrogen atoms.

The term “alicyclic” is used in connection with cyclic groups and denotes that the corresponding cyclic group is non-aromatic.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₅ alkyl” denotes an alkyl group having 1 to 5 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₄ alkyl, more preferably to methyl or ethyl, and even more preferably to methyl.

As used herein, the term “alkenyl” refers to a monovalent unsaturated acyclic hydrocarbon group which may be linear or branched and comprises one or more (e.g., one or two) carbon-to-carbon double bonds while it does not comprise any carbon-to-carbon triple bond. The term “C₂₋₅ alkenyl” denotes an alkenyl group having 2 to 5 carbon atoms. Preferred exemplary alkenyl groups are ethenyl, propenyl (e.g., prop-1-en-1-yl, prop-1-en-2-yl, or prop-2-en-1-yl), butenyl, butadienyl (e.g., buta-1,3-dien-1-yl or buta-1,3-dien-2-yl), pentenyl, or pentadienyl (e.g., isoprenyl). Unless defined otherwise, the term “alkenyl” preferably refers to C₂₋₄ alkenyl.

As used herein, the term “alkynyl” refers to a monovalent unsaturated acyclic hydrocarbon group which may be linear or branched and comprises one or more (e.g., one or two) carbon-to-carbon triple bonds and optionally one or more (e.g., one or two) carbon-to-carbon double bonds. The term “C₂₋₅ alkynyl” denotes an alkynyl group having 2 to 5 carbon atoms. Preferred exemplary alkynyl groups are ethynyl, propynyl (e.g., propargyl), or butynyl. Unless defined otherwise, the term “alkynyl” preferably refers to C₂₋₄ alkynyl.

As used herein, the term “alkylene” refers to an alkanediyl group, i.e. a divalent saturated acyclic hydrocarbon group which may be linear or branched. A “C₁₋₅ alkylene” denotes an alkylene group having 1 to 5 carbon atoms, and the term “C₀₋₃ alkylene” indicates that a covalent bond (corresponding to the option “Co alkylene”) or a C₁₋₃ alkylene is present. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₄ alkylene (including, in particular, linear C₁₋₄ alkylene), more preferably to methylene or ethylene, and even more preferably to methylene.

As used herein, the term “alkenylene” refers to an alkenediyl group, i.e. a divalent unsaturated acyclic hydrocarbon group which may be linear or branched and comprises one or more (e.g., one or two) carbon-to-carbon double bonds while it does not comprise any carbon-to-carbon triple bond. A “C₂₋₅ alkenylene” denotes an alkenylene group having 2 to 5 carbon atoms. Unless defined otherwise, the term “alkenylene” preferably refers to C₂₋₄ alkenylene (including, in particular, linear C₂₋₄ alkenylene).

As used herein, the term “alkynylene” refers to an alkynediyl group, i.e. a divalent unsaturated acyclic hydrocarbon group which may be linear or branched and comprises one or more (e.g., one or two) carbon-to-carbon triple bonds and optionally one or more (e.g., one or two) carbon-to-carbon double bonds. A “C₂₋₅ alkynylene” denotes an alkynylene group having 2 to 5 carbon atoms. Unless defined otherwise, the term “alkynylene” preferably refers to C₂₋₄ alkynylene (including, in particular, linear C₂₋₄ alkynylene).

As used herein, the term “carbocyclyl” (or “carbocyclic ring”) refers to a hydrocarbon ring group, including monocyclic rings as well as bridged ring, spiro ring and/or fused ring systems (which may be composed, e.g., of two or three rings), wherein said ring group may be saturated, partially unsaturated (i.e., unsaturated but not aromatic) or aromatic. Unless defined otherwise, “carbocyclyl” preferably refers to aryl, cycloalkyl or cycloalkenyl.

As used herein, the term “heterocyclyl” (or “heterocyclic ring”) refers to a ring group, including monocyclic rings as well as bridged ring, spiro ring and/or fused ring systems (which may be composed, e.g., of two or three rings), wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be saturated, partially unsaturated (i.e., unsaturated but not aromatic) or aromatic. For example, each heteroatom-containing ring comprised in said ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. Unless defined otherwise, “heterocyclyl” preferably refers to heteroaryl, heterocycloalkyl or heterocycloalkenyl.

As used herein, the term “aryl” refers to an aromatic hydrocarbon ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic). If the aryl is a bridged and/or fused ring system which contains, besides one or more aromatic rings, at least one non-aromatic ring (e.g., a saturated ring or an unsaturated alicyclic ring), then one or more carbon ring atoms in each non-aromatic ring may optionally be oxidized (i.e., to form an oxo group). “Aryl” may, e.g., refer to phenyl, naphthyl, dialinyl (i.e., 1,2-dihydronaphthyl), tetralinyl (i.e., 1,2,3,4-tetrahydronaphthyl), indanyl, indenyl (e.g., 1H-indenyl), anthracenyl, phenanthrenyl, 9H-fluorenyl, or azulenyl. Unless defined otherwise, an “aryl” preferably has 6 to 14 ring atoms, more preferably 6 to 10 ring atoms, even more preferably refers to phenyl or naphthyl, and most preferably refers to phenyl.

As used herein, the term “heteroaryl” (or “heteroaromatic ring”) refers to an aromatic ring group, including monocyclic aromatic rings as well as bridged ring and/or fused ring systems containing at least one aromatic ring (e.g., ring systems composed of two or three fused rings, wherein at least one of these fused rings is aromatic; or bridged ring systems composed of two or three rings, wherein at least one of these bridged rings is aromatic), wherein said aromatic ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said aromatic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heteroaryl” may, e.g., refer to thienyl (i.e., thiophenyl), benzo[b]thienyl, naphtho[2,3-b]thienyl, thianthrenyl, furyl (i.e., furanyl), benzofuranyl, isobenzofuranyl, chromanyl, chromenyl (e.g., 2H-1-benzopyranyl or 4H-1-benzopyranyl), isochromenyl (e.g., 1H-2-benzopyranyl), chromonyl, xanthenyl, phenoxathiinyl, pyrrolyl (e.g., 1H-pyrrolyl), imidazolyl, pyrazolyl, pyridyl (i.e., pyridinyl; e.g., 2-pyridyl, 3-pyridyl, or 4-pyridyl), pyrazinyl, pyrimidinyl, pyridazinyl, indolyl (e.g., 1H-indolyl), isoindolyl, indazolyl, indolizinyl, purinyl, quinolyl, isoquinolyl, phthalazinyl, naphthyridinyl, quinoxalinyl, cinnolinyl, pteridinyl, carbazolyl, β-carbolinyl, phenanthridinyl, acridinyl, perimidinyl, phenanthrolinyl (e.g., [1,10]phenanthrolinyl, [1,7]phenanthrolinyl, or [4,7]phenanthrolinyl), phenazinyl, thiazolyl, isothiazolyl, phenothiazinyl, oxazolyl, isoxazolyl, oxadiazolyl (e.g., 1,2,4-oxadiazolyl, 1,2,5-oxadiazolyl (i.e., furazanyl), or 1,3,4-oxadiazolyl), thiadiazolyl (e.g., 1,2,4-thiadiazolyl, 1,2,5-thiadiazolyl, or 1,3,4-thiadiazolyl), phenoxazinyl, pyrazolo[1,5-a]pyrimidinyl (e.g., pyrazolo[1,5-a]pyrimidin-3-yl), 1,2-benzoisoxazol-3-yl, benzothiazolyl, benzothiadiazolyl, benzoxazolyl, benzisoxazolyl, benzimidazolyl, benzo[b]thiophenyl (i.e., benzothienyl), triazolyl (e.g., 1H-1,2,3-triazolyl, 2H-1,2,3-triazolyl, 1H-1,2,4-triazolyl, or 4H-1,2,4-triazolyl), benzotriazolyl, 1H-tetrazolyl, 2H-tetrazolyl, triazinyl (e.g., 1,2,3-triazinyl, 1,2,4-triazinyl, or 1,3,5-triazinyl), furo[2,3-c]pyridinyl, dihydrofuropyridinyl (e.g., 2,3-dihydrofuro[2,3-c]pyridinyl or 1,3-dihydrofuro[3,4-c]pyridinyl), imidazopyridinyl (e.g., imidazo[1,2-a]pyridinyl or imidazo[3,2-a]pyridinyl), quinazolinyl, thienopyridinyl, tetrahydrothienopyridinyl (e.g., 4,5,6,7-tetrahydrothieno[3,2-c]pyridinyl), dibenzofuranyl, 1,3-benzodioxolyl, benzodioxanyl (e.g., 1,3-benzodioxanyl or 1,4-benzodioxanyl), or coumarinyl. Unless defined otherwise, the term “heteroaryl” preferably refers to a 5 to 14 membered (more preferably 5 to 10 membered) monocyclic ring or fused ring system comprising one or more (e.g., one, two, three or four) ring heteroatoms independently selected from O, S and N, wherein one or more S ring atoms (if present) and/or one or more N ring atoms (if present) are optionally oxidized, and wherein one or more carbon ring atoms are optionally oxidized; even more preferably, a “heteroaryl” refers to a 5 or 6 membered monocyclic ring comprising one or more (e.g., one, two or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heteroaryl” include pyridinyl (e.g., 2-pyridyl, 3-pyridyl, or 4-pyridyl), imidazolyl, thiazolyl, 1H-tetrazolyl, 2H-tetrazolyl, thienyl (i.e., thiophenyl), or pyrimidinyl.

As used herein, the term “cycloalkyl” refers to a saturated hydrocarbon ring group, including monocyclic rings as well as bridged ring, spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, decalinyl (i.e., decahydronaphthyl), or adamantyl. Unless defined otherwise, “cycloalkyl” preferably refers to a C₃₁₁ cycloalkyl, and more preferably refers to a C₃₋₇ cycloalkyl. A particularly preferred “cycloalkyl” is a monocyclic saturated hydrocarbon ring having 3 to 7 ring members. Moreover, unless defined otherwise, particularly preferred examples of a “cycloalkyl” include cyclohexyl or cyclopropyl, particularly cyclohexyl.

As used herein, the term “heterocycloalkyl” refers to a saturated ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, and further wherein one or more carbon ring atoms may optionally be oxidized (i.e., to form an oxo group). For example, each heteroatom-containing ring comprised in said saturated ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkyl” may, e.g., refer to aziridinyl, azetidinyl, pyrrolidinyl, imidazolidinyl, pyrazolidinyl, piperidinyl, piperazinyl, piperazinonyl (e.g., piperazin-2-on-1-yl or piperazin-3-on-1-yl), azepanyl, diazepanyl (e.g., 1,4-diazepanyl), oxazolidinyl, isoxazolidinyl, thiazolidinyl, isothiazolidinyl, morpholinyl (e.g., morpholin-4-yl), thiomorpholinyl (e.g., thiomorpholin-4-yl), oxazepanyl, oxiranyl, oxetanyl, tetrahydrofuranyl, 1,3-dioxolanyl, tetrahydropyranyl, 1,4-dioxanyl, oxepanyl, thiiranyl, thietanyl, tetrahydrothiophenyl (i.e., thiolanyl), 1,3-dithiolanyl, thianyl, thiepanyl, decahydroquinolinyl, decahydroisoquinolinyl, or 2-oxa-5-aza-bicyclo[2.2.1]hept-5-yl. Unless defined otherwise, “heterocycloalkyl” preferably refers to a 3 to 11 membered saturated ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, and wherein one or more carbon ring atoms are optionally oxidized; more preferably, “heterocycloalkyl” refers to a 5 to 7 membered saturated monocyclic ring group containing one or more (e.g., one, two, or three) ring heteroatoms independently selected from O, S and N, wherein one or more S ring atoms (if present) and/or one or more N ring atoms (if present) are optionally oxidized, and wherein one or more carbon ring atoms are optionally oxidized. Moreover, unless defined otherwise, particularly preferred examples of a “heterocycloalkyl” include tetrahydropyranyl, piperidinyl, piperazinyl, piperazinonyl, morpholinyl, pyrrolidinyl, or tetrahydrofuranyl.

As used herein, the term “cycloalkenyl” refers to an unsaturated alicyclic (non-aromatic) hydrocarbon ring group, including monocyclic rings as well as bridged ring, spiro ring and/or fused ring systems (which may be composed, e.g., of two or three rings; such as, e.g., a fused ring system composed of two or three fused rings), wherein said hydrocarbon ring group comprises one or more (e.g., one or two) carbon-to-carbon double bonds and does not comprise any carbon-to-carbon triple bond. “Cycloalkenyl” may, e.g., refer to cyclopropenyl, cyclobutenyl, cyclopentenyl, cyclohexenyl, cyclohexadienyl, cycloheptenyl, or cycloheptadienyl. Unless defined otherwise, “cycloalkenyl” preferably refers to a C₃₁₁ cycloalkenyl, and more preferably refers to a C₃₋₇ cycloalkenyl. A particularly preferred “cycloalkenyl” is a monocyclic unsaturated alicyclic hydrocarbon ring having 3 to 7 ring members and containing one or more (e.g., one or two; preferably one) carbon-to-carbon double bonds.

As used herein, the term “heterocycloalkenyl” refers to an unsaturated alicyclic (non-aromatic) ring group, including monocyclic rings as well as bridged ring, spiro ring and/or fused ring systems (which may be composed, e.g., of two or three rings; such as, e.g., a fused ring system composed of two or three fused rings), wherein said ring group contains one or more (such as, e.g., one, two, three, or four) ring heteroatoms independently selected from 0, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group comprises at least one double bond between adjacent ring atoms and does not comprise any triple bond between adjacent ring atoms. For example, each heteroatom-containing ring comprised in said unsaturated alicyclic ring group may contain one or two O atoms and/or one or two S atoms (which may optionally be oxidized) and/or one, two, three or four N atoms (which may optionally be oxidized), provided that the total number of heteroatoms in the corresponding heteroatom-containing ring is 1 to 4 and that there is at least one carbon ring atom (which may optionally be oxidized) in the corresponding heteroatom-containing ring. “Heterocycloalkenyl” may, e.g., refer to imidazolinyl (e.g., 2-imidazolinyl (i.e., 4,5-dihydro-1H-imidazolyl), 3-imidazolinyl, or 4-imidazolinyl), tetrahydropyridinyl (e.g., 1,2,3,6-tetrahydropyridinyl), dihydropyridinyl (e.g., 1,2-dihydropyridinyl or 2,3-dihydropyridinyl), pyranyl (e.g., 2H-pyranyl or 4H-pyranyl), thiopyranyl (e.g., 2H-thiopyranyl or 4H-thiopyranyl), dihydropyranyl, dihydrofuranyl, dihydropyrazolyl, dihydropyrazinyl, dihydroisoindolyl, octahydroquinolinyl (e.g., 1,2,3,4,4a,5,6,7-octahydroquinolinyl), or octahydroisoquinolinyl (e.g., 1,2,3,4,5,6,7,8-octahydroisoquinolinyl). Unless defined otherwise, “heterocycloalkenyl” preferably refers to a 3 to 11 membered unsaturated alicyclic ring group, which is a monocyclic ring or a fused ring system (e.g., a fused ring system composed of two fused rings), wherein said ring group contains one or more (e.g., one, two, three, or four) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms; more preferably, “heterocycloalkenyl” refers to a 5 to 7 membered monocyclic unsaturated non-aromatic ring group containing one or more (e.g., one, two, or three) ring heteroatoms independently selected from O, S and N, wherein one or more S ring atoms (if present) and/or one or more N ring atoms (if present) are optionally oxidized, wherein one or more carbon ring atoms are optionally oxidized, and wherein said ring group comprises at least one double bond between adjacent ring atoms and does not comprise any triple bond between adjacent ring atoms.

As used herein, the term “halogen” refers to fluoro (—F), chloro (—Cl), bromo (—Br), or iodo (—I).

As used herein, the term “haloalkyl” refers to an alkyl group substituted with one or more (preferably 1 to 6, more preferably 1 to 3) halogen atoms which are selected independently from fluoro, chloro, bromo and iodo, and are preferably all fluoro atoms. It will be understood that the maximum number of halogen atoms is limited by the number of available attachment sites and, thus, depends on the number of carbon atoms comprised in the alkyl moiety of the haloalkyl group. “Haloalkyl” may, e.g., refer to —CF₃, —CHF₂, —CH₂F, —CF₂—CH₃, —CH₂—CF₃, —CH₂—CHF₂, —CH₂—CF₂—CH₃, —CH₂—CF₂—CF₃, or —CH(CF₃)₂. A particularly preferred “haloalkyl” group is —CF₃.

The terms “bond” and “covalent bond” are used herein synonymously, unless explicitly indicated otherwise or contradicted by context.

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

Various groups are referred to as being “optionally substituted” in this specification. Generally, these groups may carry one or more substituents, such as, e.g., one, two, three or four substituents. It will be understood that the maximum number of substituents is limited by the number of attachment sites available on the substituted moiety. Unless defined otherwise, the “optionally substituted” groups referred to in this specification carry preferably not more than two substituents and may, in particular, carry only one substituent. Moreover, unless defined otherwise, it is preferred that the optional substituents are absent, i.e. that the corresponding groups are unsubstituted.

A skilled person will appreciate that the substituent groups comprised in the compounds of the present invention may be attached to the remainder of the respective compound via a number of different positions of the corresponding specific substituent group. Unless defined otherwise, the preferred attachment positions for the various specific substituent groups are as illustrated in the examples.

As used herein, unless explicitly indicated otherwise or contradicted by context, the terms “a”, “an” and “the” are used interchangeably with “one or more” and “at least one”. Thus, for example, a composition comprising “a” compound of formula (I) can be interpreted as referring to a composition comprising “one or more” compounds of formula (I).

It is to be understood that wherever numerical ranges are provided/disclosed herein, all values and subranges encompassed by the respective numerical range are meant to be encompassed within the scope of the invention. Accordingly, the present invention specifically and individually relates to each value that falls within a numerical range disclosed herein, as well as each subrange encompassed by a numerical range disclosed herein.

As used herein, the term “about” preferably refers to ±10% of the indicated numerical value, more preferably to ±5% of the indicated numerical value, and in particular to the exact numerical value indicated. If the term “about” is used in connection with the endpoints of a range, it preferably refers to the range from the lower endpoint −10% of its indicated numerical value to the upper endpoint +10% of its indicated numerical value, more preferably to the range from of the lower endpoint −5% to the upper endpoint +5%, and even more preferably to the range defined by the exact numerical values of the lower endpoint and the upper endpoint.

As used herein, the term “comprising” (or “comprise”, “comprises”, “contain”, “contains”, or “containing”), unless explicitly indicated otherwise or contradicted by context, has the meaning of “containing, inter alia”, i.e., “containing, among further optional elements, . . . ”. In addition thereto, this term also includes the narrower meanings of “consisting essentially of” and “consisting of”. For example, the term “A comprising B and C” has the meaning of “A containing, inter alia, B and C”, wherein A may contain further optional elements (e.g., “A containing B, C and D” would also be encompassed), but this term also includes the meaning of “A consisting essentially of B and C” and the meaning of “A consisting of B and C” (i.e., no other components than B and C are comprised in A).

The scope of the invention embraces all pharmaceutically acceptable salt forms of the compounds of formula (I) which may be formed, e.g., by protonation of an atom carrying an electron lone pair which is susceptible to protonation, such as an amino group, with an inorganic or organic acid, or as a salt of an acid group (such as a carboxylic acid group) with a physiologically acceptable cation. Exemplary base addition salts comprise, for example: alkali metal salts such as sodium or potassium salts; alkaline earth metal salts such as calcium or magnesium salts; zinc salts; ammonium salts; aliphatic amine salts such as trimethylamine, triethylamine, dicyclohexylamine, ethanolamine, diethanolamine, triethanolamine, procaine salts, meglumine salts, ethylenediamine salts, or choline salts; aralkyl amine salts such as N,N-dibenzylethylenediamine salts, benzathine salts, benethamine salts; heterocyclic aromatic amine salts such as pyridine salts, picoline salts, quinoline salts or isoquinoline salts; quaternary ammonium salts such as tetramethylammonium salts, tetraethylammonium salts, benzyltrimethylammonium salts, benzyltriethylammonium salts, benzyltributylammonium salts, methyltrioctylammonium salts or tetrabutylammonium salts; and basic amino acid salts such as arginine salts, lysine salts, or histidine salts. Exemplary acid addition salts comprise, for example: mineral acid salts such as hydrochloride, hydrobromide, hydroiodide, sulfate salts (such as, e.g., sulfate or hydrogensulfate salts), nitrate salts, phosphate salts (such as, e.g., phosphate, hydrogenphosphate, or dihydrogenphosphate salts), carbonate salts, hydrogencarbonate salts, perchlorate salts, borate salts, or thiocyanate salts; organic acid salts such as acetate, propionate, butyrate, pentanoate, hexanoate, heptanoate, octanoate, cyclopentanepropionate, decanoate, undecanoate, oleate, stearate, lactate, maleate, oxalate, fumarate, tartrate, malate, citrate, succinate, adipate, gluconate, glycolate, nicotinate, benzoate, salicylate, ascorbate, pamoate (embonate), camphorate, glucoheptanoate, or pivalate salts; sulfonate salts such as methanesulfonate (mesylate), ethanesulfonate (esylate), 2-hydroxyethanesulfonate (isethionate), benzenesulfonate (besylate), p-toluenesulfonate (tosylate), 2-naphthalenesulfonate (napsylate), 3-phenylsulfonate, or camphorsulfonate salts; glycerophosphate salts; and acidic amino acid salts such as aspartate or glutamate salts. Further pharmaceutically acceptable salts are described in the literature, e.g., in Stahl P H & Wermuth C G (eds.), “Handbook of Pharmaceutical Salts: Properties, Selection, and Use”, Wiley-VCH, 2002 and in the references cited therein. Preferred pharmaceutically acceptable salts of the compounds of formula (I) include a hydrochloride salt, a hydrobromide salt, a mesylate salt, a sulfate salt, a tartrate salt, a fumarate salt, an acetate salt, a citrate salt, and a phosphate salt. A particularly preferred pharmaceutically acceptable salt of the compound of formula (I) is a hydrochloride salt. Accordingly, it is preferred that the compound of formula (I), including any one of the specific compounds of formula (I) described herein, is in the form of a hydrochloride salt, a hydrobromide salt, a mesylate salt, a sulfate salt, a tartrate salt, a fumarate salt, an acetate salt, a citrate salt, or a phosphate salt, and it is particularly preferred that the compound of formula (I) is in the form of a hydrochloride salt.

The present invention also specifically relates to the compound of formula (I), including any one of the specific compounds of formula (I) described herein, in non-salt form.

Moreover, the scope of the invention embraces the compounds of formula (I) in any solvated form, including, e.g., solvates with water (i.e., as a hydrate) or solvates with organic solvents such as, e.g., methanol, ethanol, isopropanol, acetic acid, ethyl acetate, ethanolamine, DMSO, or acetonitrile. All physical forms, including any amorphous or crystalline forms (i.e., polymorphs), of the compounds of formula (I) are also encompassed within the scope of the invention. It is to be understood that such solvates and physical forms of pharmaceutically acceptable salts of the compounds of the formula (I) are likewise embraced by the invention.

Furthermore, the compounds of formula (I) may exist in the form of different isomers, in particular stereoisomers (including, e.g., geometric isomers (or cis/trans isomers), enantiomers and diastereomers) or tautomers (including, in particular, prototropic tautomers, such as keto/enol tautomers or thione/thiol tautomers). All such isomers of the compounds of formula (I) are contemplated as being part of the present invention, either in admixture or in pure or substantially pure form. As for stereoisomers, the invention embraces the isolated optical isomers of the compounds according to the invention as well as any mixtures thereof (including, in particular, racemic mixtures/racemates). The racemates can be resolved by physical methods, such as, e.g., fractional crystallization, separation or crystallization of diastereomeric derivatives, or separation by chiral column chromatography. The individual optical isomers can also be obtained from the racemates via salt formation with an optically active acid followed by crystallization. The present invention further encompasses any tautomers of the compounds of formula (I). It will be understood that some compounds may exhibit tautomerism. In such cases, the formulae provided herein expressly depict only one of the possible tautomeric forms. The formulae and chemical names as provided herein are intended to encompass any tautomeric form of the corresponding compound and not to be limited merely to the specific tautomeric form depicted by the drawing or identified by the name of the compound.

The scope of the invention also embraces compounds of formula (I), in which one or more atoms are replaced by a specific isotope of the corresponding atom. For example, the invention encompasses compounds of formula (I), in which one or more hydrogen atoms (or, e.g., all hydrogen atoms) are replaced by deuterium atoms (i.e., ²H; also referred to as “D”). Accordingly, the invention also embraces compounds of formula (I) which are enriched in deuterium. Naturally occurring hydrogen is an isotopic mixture comprising about 99.98 mol-% hydrogen-1 (¹H) and about 0.0156 mol-% deuterium (²H or D). The content of deuterium in one or more hydrogen positions in the compounds of formula (I) can be increased using deuteration techniques known in the art. For example, a compound of formula (I) or a reactant or precursor to be used in the synthesis of the compound of formula (I) can be subjected to an H/D exchange reaction using, e.g., heavy water (D₂O). Further suitable deuteration techniques are described in: Atzrodt J et al., Bioorg Med Chem, 20(18), 5658-5667, 2012; William J S et al., Journal of Labelled Compounds and Radiopharmaceuticals, 53(11-12), 635-644, 2010; Modvig A et al., J Org Chem, 79, 5861-5868, 2014. The content of deuterium can be determined, e.g., using mass spectrometry or NMR spectroscopy. Unless specifically indicated otherwise, it is preferred that the compound of formula (I) is not enriched in deuterium. Accordingly, the presence of naturally occurring hydrogen atoms or ¹H hydrogen atoms in the compounds of formula (I) is preferred.

The present invention also embraces compounds of formula (I), in which one or more atoms are replaced by a positron-emitting isotope of the corresponding atom, such as, e.g., ¹⁸F, ¹¹C, ¹³N, ¹⁵O, ⁷⁶Br, ⁷⁷Br, ¹²⁰I and/or ¹²⁴I. Such compounds can be used as tracers, trackers or imaging probes in positron emission tomography (PET). The invention thus includes (i) compounds of formula (I), in which one or more fluorine atoms (or, e.g., all fluorine atoms) are replaced by ¹⁸F atoms, (ii) compounds of formula (I), in which one or more carbon atoms (or, e.g., all carbon atoms) are replaced by ¹¹C atoms, (iii) compounds of formula (I), in which one or more nitrogen atoms (or, e.g., all nitrogen atoms) are replaced by ¹³N atoms, (iv) compounds of formula (I), in which one or more oxygen atoms (or, e.g., all oxygen atoms) are replaced by ¹⁵O atoms, (v) compounds of formula (I), in which one or more bromine atoms (or, e.g., all bromine atoms) are replaced by ⁷⁶Br atoms, (vi) compounds of formula (I), in which one or more bromine atoms (or, e.g., all bromine atoms) are replaced by ⁷⁷Br atoms, (vii) compounds of formula (I), in which one or more iodine atoms (or, e.g., all iodine atoms) are replaced by ¹²⁰I atoms, and (viii) compounds of formula (I), in which one or more iodine atoms (or, e.g., all iodine atoms) are replaced by ¹²⁴I atoms. In general, it is preferred that none of the atoms in the compounds of formula (I) are replaced by specific isotopes.

The compounds provided herein may be administered as compounds per se or may be formulated as medicaments. The medicaments/pharmaceutical compositions may optionally comprise one or more pharmaceutically acceptable excipients, such as carriers, diluents, fillers, disintegrants, lubricating agents, binders, colorants, pigments, stabilizers, preservatives, antioxidants, and/or solubility enhancers.

The pharmaceutical compositions may comprise one or more solubility enhancers, such as, e.g., poly(ethylene glycol), including poly(ethylene glycol) having a molecular weight in the range of about 200 to about 5,000 Da (e.g., PEG 200, PEG 300, PEG 400, or PEG 600), ethylene glycol, propylene glycol, glycerol, a non-ionic surfactant, tyloxapol, polysorbate 80, macrogol-15-hydroxystearate (e.g., Kolliphor® HS 15, CAS 70142-34-6), a phospholipid, lecithin, dimyristoyl phosphatidylcholine, dipalmitoyl phosphatidylcholine, distearoyl phosphatidylcholine, a cyclodextrin, α-cyclodextrin, β-cyclodextrin, γ-cyclodextrin, hydroxyethyl-β-cyclodextrin, hydroxypropyl-β-cyclodextrin, hydroxyethyl-γ-cyclodextrin, hydroxypropyl-γ-cyclodextrin, dihydroxypropyl-β-cyclodextrin, sulfobutylether-β-cyclodextrin, sulfobutylether-γ-cyclodextrin, glucosyl-α-cyclodextrin, glucosyl-β-cyclodextrin, diglucosyl-β-cyclodextrin, maltosyl-α-cyclodextrin, maltosyl-β-cyclodextrin, maltosyl-γ-cyclodextrin, maltotriosyl-β-cyclodextrin, maltotriosyl-γ-cyclodextrin, dimaltosyl-β-cyclodextrin, methyl-β-cyclodextrin, a carboxyalkyl thioether, hydroxypropyl methylcellulose, hydroxypropylcellulose, polyvinylpyrrolidone, a vinyl acetate copolymer, vinyl pyrrolidone, sodium lauryl sulfate, dioctyl sodium sulfosuccinate, or any combination thereof.

The pharmaceutical compositions may also comprise one or more preservatives, particularly one or more antimicrobial preservatives, such as, e.g., benzyl alcohol, chlorobutanol, 2-ethoxyethanol, m-cresol, chlorocresol (e.g., 2-chloro-3-methyl-phenol or 4-chloro-3-methyl-phenol), benzalkonium chloride, benzethonium chloride, benzoic acid (or a pharmaceutically acceptable salt thereof), sorbic acid (or a pharmaceutically acceptable salt thereof), chlorhexidine, thimerosal, or any combination thereof.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

The compounds of formula (I) or the pharmaceutically acceptable salts or solvates thereof, or the above described pharmaceutical compositions comprising any of the aforementioned entities, may be administered to a subject by any convenient route of administration, whether systemically/peripherally or at the site of desired action, including but not limited to one or more of: oral (e.g., as a tablet, capsule, or as an ingestible solution), topical (e.g., transdermal, intranasal, ocular, buccal, and sublingual), parenteral (e.g., using injection techniques or infusion techniques, and including, for example, by injection, e.g., subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, or intrasternal by, e.g., implant of a depot, for example, subcutaneously or intramuscularly), pulmonary (e.g., by inhalation or insufflation therapy using, e.g., an aerosol, e.g., through mouth or nose), gastrointestinal, intrauterine, intraocular, subcutaneous, ophthalmic (including intravitreal or intracameral), rectal, or vaginal administration.

If said compounds or pharmaceutical compositions are administered parenterally, then examples of such administration include one or more of: intravenously, intraarterially, intraperitoneally, intrathecally, intraventricularly, intraurethrally, intrasternally, intracardially, intracranially, intramuscularly or subcutaneously administering the compounds or pharmaceutical compositions, and/or by using infusion techniques. For parenteral administration, the compounds are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Said compounds or pharmaceutical compositions can also be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included. Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

For oral administration, the compounds or pharmaceutical compositions are preferably administered by oral ingestion, particularly by swallowing. The compounds or pharmaceutical compositions can thus be administered to pass through the mouth into the gastrointestinal tract, which can also be referred to as “oral-gastrointestinal” administration.

Alternatively, said compounds or pharmaceutical compositions can be administered in the form of a suppository or pessary, or may be applied topically in the form of a gel, hydrogel, lotion, solution, cream, ointment or dusting powder. The compounds of the present invention may also be dermally or transdermally administered, for example, by the use of a skin patch.

Said compounds or pharmaceutical compositions may also be administered by sustained release systems. Suitable examples of sustained-release compositions include semi-permeable polymer matrices in the form of shaped articles, e.g., films, or microcapsules. Sustained-release matrices include, e.g., polylactides, copolymers of L-glutamic acid and gamma-ethyl-L-glutamate, poly(2-hydroxyethyl methacrylate), ethylene vinyl acetate, or poly-D-(−)-3-hydroxybutyric acid. Sustained-release pharmaceutical compositions also include liposomally entrapped compounds. The present invention thus also relates to liposomes containing a compound of the invention.

Said compounds or pharmaceutical compositions may also be administered by the pulmonary route, rectal routes, or the ocular route. For ophthalmic use, they can be formulated as micronized suspensions in isotonic, pH adjusted, sterile saline, or, preferably, as solutions in isotonic, pH adjusted, sterile saline, optionally in combination with a preservative such as a benzalkonium chloride. Alternatively, they may be formulated in an ointment such as petrolatum.

It is also envisaged to prepare dry powder formulations of the compounds of formula (I) for pulmonary administration, particularly inhalation. Such dry powders may be prepared by spray drying under conditions which result in a substantially amorphous glassy or a substantially crystalline bioactive powder. Accordingly, dry powders of the compounds of the present invention can be made according to an emulsification/spray drying process.

For topical application to the skin, said compounds or pharmaceutical compositions can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, 2-octyldodecanol, benzyl alcohol and water.

The present invention thus relates to the compounds or the pharmaceutical compositions provided herein, wherein the corresponding compound or pharmaceutical composition is to be administered by any one of: an oral route; topical route, including by transdermal, intranasal, ocular, buccal, or sublingual route; parenteral route using injection techniques or infusion techniques, including by subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, intrasternal, intraventricular, intraurethral, or intracranial route; pulmonary route, including by inhalation or insufflation therapy; gastrointestinal route; intrauterine route; intraocular route; subcutaneous route; ophthalmic route, including by intravitreal, or intracameral route; rectal route; or vaginal route. Preferred routes of administration are oral administration or parenteral administration. For each of the compounds or pharmaceutical compositions provided herein, it is particularly preferred that the respective compound or pharmaceutical composition is to be administered orally (particularly by oral ingestion).

Typically, a physician will determine the actual dosage which will be most suitable for an individual subject. The specific dose level and frequency of dosage for any particular individual subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual subject undergoing therapy.

A proposed, yet non-limiting dose of the compounds according to the invention for oral administration to a human (of approximately 70 kg body weight) may be 0.05 to 2000 mg, preferably 0.1 mg to 1000 mg, of the active ingredient per unit dose. The unit dose may be administered, e.g., 1 to 3 times per day. The unit dose may also be administered 1 to 7 times per week, e.g., with not more than one administration per day. It will be appreciated that it may be necessary to make routine variations to the dosage depending on the age and weight of the patient/subject as well as the severity of the condition to be treated. The precise dose and also the route of administration will ultimately be at the discretion of the attendant physician or veterinarian.

The compound of formula (I) or the pharmaceutically acceptable salt or solvate thereof, or a pharmaceutical composition comprising any of the aforementioned entities, can be administered in monotherapy (e.g., without concomitantly administering any further therapeutic agents, or without concomitantly administering any further therapeutic agents against the same disease that is to be treated or prevented with the compound of formula (I)). However, the compound of formula (I) or the pharmaceutically acceptable salt or solvate thereof, or a pharmaceutical composition comprising any of the aforementioned entities, can also be administered in combination with one or more further therapeutic agents. If the compound of formula (I) is used in combination with a second therapeutic agent active against the same disease or condition, the dose of each compound may differ from that when the corresponding compound is used alone, in particular, a lower dose of each compound may be used. The combination of the compound of formula (I) with one or more further therapeutic agents may comprise the simultaneous/concomitant administration of the compound of formula (I) and the further therapeutic agent(s) (either in a single pharmaceutical formulation or in separate pharmaceutical formulations), or the sequential/separate administration of the compound of formula (I) and the further therapeutic agent(s). If administration is sequential, either the compound of formula (I) according to the invention or the one or more further therapeutic agents may be administered first. If administration is simultaneous, the one or more further therapeutic agents may be included in the same pharmaceutical formulation as the compound of formula (I), or they may be administered in two or more different (separate) pharmaceutical formulations.

For the treatment or prevention of cancer, the one or more further therapeutic agents to be administered in combination with a compound of the present invention are preferably anticancer drugs. The anticancer drug(s) to be administered in combination with a compound of formula (I) or a pharmaceutically acceptable salt or solvate thereof may, e.g., be selected from: a tumor angiogenesis inhibitor (e.g., a protease inhibitor, an epidermal growth factor receptor kinase inhibitor, or a vascular endothelial growth factor receptor kinase inhibitor); a cytotoxic drug (e.g., an antimetabolite, such as purine and pyrimidine analog antimetabolites); an antimitotic agent (e.g., a microtubule stabilizing drug or an antimitotic alkaloid); a platinum coordination complex; an anti-tumor antibiotic; an alkylating agent (e.g., a nitrogen mustard or a nitrosourea); an endocrine agent (e.g., an adrenocorticosteroid, an androgen, an anti-androgen, an estrogen, an anti-estrogen, an aromatase inhibitor, a gonadotropin-releasing hormone agonist, or a somatostatin analog); a compound that targets an enzyme or receptor that is overexpressed and/or otherwise involved in a specific metabolic pathway that is deregulated (or misregulated) in the tumor cell (e.g., ATP and GTP phosphodiesterase inhibitors, histone deacetylase inhibitors, protein kinase inhibitors (such as serine, threonine and tyrosine kinase inhibitors, e.g., Abelson protein tyrosine kinase inhibitors) and the various growth factors, their receptors and corresponding kinase inhibitors (such as epidermal growth factor receptor kinase inhibitors, vascular endothelial growth factor receptor kinase inhibitors, fibroblast growth factor inhibitors, insulin-like growth factor receptor inhibitors and platelet-derived growth factor receptor kinase inhibitors)); methionine; an aminopeptidase inhibitor; a proteasome inhibitor; a cyclooxygenase inhibitor (e.g., a cyclooxygenase-1 inhibitor or a cyclooxygenase-2 inhibitor); a topoisomerase inhibitor (e.g., a topoisomerase I inhibitor or a topoisomerase II inhibitor); a poly ADP ribose polymerase inhibitor (PARP inhibitor); an epidermal growth factor receptor (EGFR) inhibitor/antagonist; an adenosine A_{2A} receptor antagonist; an adenosine A_{2B} receptor antagonist; a dual adenosine A_{2A}/A_{2B} receptor antagonist; and a prostaglandin E2 receptor 4 (EP4) antagonist.

An alkylating agent which can be used as an anticancer drug in combination with a compound of the present invention may be, for example, a nitrogen mustard (such as cyclophosphamide, mechlorethamine (chlormethine), uramustine, melphalan, chlorambucil, ifosfamide, bendamustine, or trofosfamide), a nitrosourea (such as carmustine, streptozocin, fotemustine, lomustine, nimustine, prednimustine, ranimustine, or semustine), an alkyl sulfonate (such as busulfan, mannosulfan, or treosulfan), an aziridine (such as hexamethylmelamine (altretamine), triethylenemelamine, ThioTEPA (N,N′N′-triethylenethiophosphoramide), carboquone, or triaziquone), a hydrazine (such as procarbazine), a triazene (such as dacarbazine), or an imidazotetrazine (such as temozolomide).

A platinum coordination complex which can be used as an anticancer drug in combination with a compound of the present invention may be, for example, cisplatin, carboplatin, nedaplatin, oxaliplatin, satraplatin, or triplatin tetranitrate.

A cytotoxic drug which can be used as an anticancer drug in combination with a compound of the present invention may be, for example, an antimetabolite, including folic acid analogue antimetabolites (such as aminopterin, methotrexate, pemetrexed, or raltitrexed), purine analogue antimetabolites (such as cladribine, clofarabine, fludarabine, 6-mercaptopurine (including its prodrug form azathioprine), pentostatin, or 6-thioguanine), and pyrimidine analogue antimetabolites (such as cytarabine, decitabine, 5-fluorouracil (including its prodrug forms capecitabine and tegafur), floxuridine, gemcitabine, enocitabine, or sapacitabine).

An antimitotic agent which can be used as an anticancer drug in combination with a compound of the present invention may be, for example, a taxane (such as docetaxel, larotaxel, ortataxel, paclitaxel/taxol, tesetaxel, or nab-paclitaxel (e.g., Abraxane®)), a Vinca alkaloid (such as vinblastine, vincristine, vinflunine, vindesine, or vinorelbine), an epothilone (such as epothilone A, epothilone B, epothilone C, epothilone D, epothilone E, or epothilone F) or an epothilone B analogue (such as ixabepilone/azaepothilone B).

An anti-tumor antibiotic which can be used as an anticancer drug in combination with a compound of the present invention may be, for example, an anthracycline (such as aclarubicin, daunorubicin, doxorubicin, epirubicin, idarubicin, amrubicin, pirarubicin, valrubicin, or zorubicin), an anthracenedione (such as mitoxantrone, or pixantrone) or an anti-tumor antibiotic isolated from Streptomyces (such as actinomycin (including actinomycin D), bleomycin, mitomycin (including mitomycin C), or plicamycin).

A tyrosine kinase inhibitor which can be used as an anticancer drug in combination with a compound of the present invention may be, for example, axitinib, bosutinib, cediranib, dasatinib, erlotinib, gefitinib, imatinib, lapatinib, lestaurtinib, nilotinib, semaxanib, sorafenib, sunitinib, axitinib, nintedanib, ponatinib, vandetanib, or vemurafenib.

A topoisomerase inhibitor which can be used as an anticancer drug in combination with a compound of the present invention may be, for example, a topoisomerase I inhibitor (such as irinotecan, topotecan, camptothecin, belotecan, rubitecan, or lamellarin D) or a topoisomerase II inhibitor (such as amsacrine, etoposide, etoposide phosphate, teniposide, or doxorubicin).

A PARP inhibitor which can be used as an anticancer drug in combination with a compound of the present invention may be, for example, niraparib, olaparib, rucaparib, talazoparib, veliparib, pamiparib (BGB-290), BMN-673, CEP 9722, MK 4827, E7016, or 3-aminobenzamide.

An EGFR inhibitor/antagonist which can be used as an anticancer drug in combination with a compound of the present invention may be, for example, gefitinib, erlotinib, lapatinib, afatinib, neratinib, osimertinib, brigatinib, dacomitinib, vandetanib, pelitinib, canertinib, icotinib, poziotinib, ABT-414, AV-412, PD 153035, PKI-166, BMS-690514, CUDC-101, AP26113, XL647, cetuximab, panitumumab, zalutumumab, nimotuzumab, or matuzumab.

An adenosine A_{2A} receptor antagonist which can be used as an anticancer drug in combination with a compound of the present invention may be, for example, ciforadenant, imaradenant, inupadenant, istradefylline, preladenant, SCH-58261, SCH-442416, ST 1535, or ZM241385.

An adenosine A_{2B} receptor antagonist which can be used as an anticancer drug in combination with a compound of the present invention may be, for example, LAS38096 or LAS101057.

A dual adenosine A_{2A}/A_{2B} receptor antagonist which can be used as an anticancer drug in combination with a compound of the present invention may be, for example, M1069, etrumadenant, or INCB106385.

A prostaglandin E2 receptor 4 (EP4) antagonist which can be used as an anticancer drug in combination with a compound of the present invention may be, for example, DT-9081, grapiprant, palupiprant, BAY-1316957, CJ-42794, ER-819762, GW627368, L-161982, MF498, MF-766, MK-2894, or ONO-AE3-208.

Further anticancer drugs may also be used in combination with a compound of the present invention. The anticancer drugs may comprise biological or chemical molecules, like TNF-related apoptosis-inducing ligand (TRAIL), tamoxifen, amsacrine, bexarotene, estramustine, irofulven, trabectedin, cetuximab, panitumumab, tositumomab, alemtuzumab, bevacizumab, edrecolomab, gemtuzumab, alvocidib, seliciclib, aminolevulinic acid, methyl aminolevulinate, efaproxiral, porfimer sodium, talaporfin, temoporfin, verteporfin, alitretinoin, tretinoin, anagrelide, arsenic trioxide, atrasentan, bortezomib, carmofur, celecoxib, demecolcine, elesclomol, elsamitrucin, etoglucid, lonidamine, lucanthone, masoprocol, mitobronitol, mitoguazone, mitotane, oblimersen, omacetaxine, sitimagene, ceradenovec, tegafur, testolactone, tiazofurine, tipifarnib, vorinostat, iniparib, or copanlisib.

Also biological drugs, like antibodies, antibody fragments, antibody constructs (for example, single-chain constructs), and/or modified antibodies (like CDR-grafted antibodies, humanized antibodies, “fully human” antibodies, etc.) directed against cancer or tumor markers/factors/cytokines involved in proliferative diseases can be employed in cotherapy approaches with the compounds of the invention. Examples of such biological molecules are anti-HER2 antibodies (e.g. trastuzumab, Herceptin®), anti-CD20 antibodies (e.g. Rituximab, Rituxan®, MabThera®, Reditux®), anti-CD19/CD3 constructs, and anti-TNF antibodies (see, e.g., Taylor P C, Curr Opin Pharmacol, 2003, 3(3):323-328). An anticancer drug which can be used in combination with a compound of the present invention may be, in particular, an immunooncology therapeutic (such as an antibody (e.g., a monoclonal antibody or a polyclonal antibody), an antibody fragment, an antibody construct (e.g., a single-chain construct), or a modified antibody (e.g., a CDR-grafted antibody, a humanized antibody, or a “fully human” antibody) or a small molecule) targeting any one of CTLA-4, PD-1, PD-L1, TIGIT, TIM3, LAG3, OX40, CSF1R, IDO, CD40, adenosine A_{2A} receptor (A2A), adenosine A_{2B} receptor (A2B), A2A/A2B, prostaglandin E2 receptor 4 (EP4), or chemokine (C—C motif) receptor 8 (CCR8). Such immunooncology therapeutics include, e.g., an anti-CTLA-4 antibody (e.g., ipilimumab or tremelimumab), an anti-PD-1 antibody (e.g., nivolumab (BMS-936558), pembrolizumab (MK-3475), pidilizumab (CT-011), cemiplimab, dostarlimab, spartalizumab, camrelizumab, sintilimab, tislelizumab, toripalimab, zimberelimab, AMP-224, AMP-514 (or MEDI0680), JTX-4014, INCMGA00012 (or MGA012), or APE02058), an anti-PD-L1 antibody (e.g., atezolizumab, avelumab, durvalumab, KN035, CK-301, BMS-936559, MEDI4736, MPDL3280A (RG7446), MDX-1105, MEDI6469, or bintrafusp alfa), an anti-TIGIT antibody (e.g., tiragolumab, vibostolimab, domvanalimab, etigilimab, BMS-986207, EOS-448, COM902, ASP8374, SEA-TGT, BGB-A1217, IBI-939, or M6223), an anti-TIM3 antibody, an anti-LAG3 antibody (e.g., relatlimab (or BMS-986016), ieramilimab (or LAG525), encelimab (or TSR-033), tebotelimab (or MGD013), REGN3767 (or R3767), FS118, IMP701, or IMP731), an anti-OX40 antibody (e.g., MEDI0562), an anti-CSF1R antibody (e.g., IMC-CS4 or RG7155), an anti-IDO antibody, an anti-CD40 antibody (e.g., CP-870,893 or Chi Lob 7/4), or an anti-CCR8 antibody (e.g., DT-7012, BMS-986340, S-531011, BAY-3375968, GS-1811 (or JTX-1811), FPA157, SRF114, HBM1022, or LM-108). Further immunooncology therapeutics are known in the art and are described, e.g., in: Kyi C et al., FEBS Lett, 2014, 588(2):368-76; Intlekofer A M et al., J Leukoc Biol, 2013, 94(1):25-39; Callahan M K et al., J Leukoc Biol, 2013, 94(1):41-53; Ngiow S F et al., Cancer Res, 2011, 71(21):6567-71; and Blattman J N et al., Science, 2004, 305(5681):200-5.

In particular, a compound of formula (I) or a pharmaceutically acceptable salt or solvate thereof, or a pharmaceutical composition comprising any of the aforementioned entities, may be administered in combination with an immune checkpoint inhibitor, preferably an antibody (or an antigen-binding fragment thereof, or an antibody construct) directed against CTLA-4, PD-1, PD-L1, TIGIT, or LAG3. Corresponding preferred examples include, but are not limited to, any one of the anti-CTLA-4 antibodies ipilimumab or tremelimumab, any one of the anti-PD-1 antibodies nivolumab, pembrolizumab, pidilizumab, cemiplimab, dostarlimab, spartalizumab, camrelizumab, sintilimab, tislelizumab, toripalimab, zimberelimab, AMP-224, AMP-514, JTX-4014, INCMGA00012, or APE02058, any one of the anti-PD-L1 antibodies atezolizumab, avelumab, durvalumab, KN035, CK-301, BMS-936559, MEDI4736, MPDL3280A, MDX-1105, MEDI6469 or bintrafusp alfa, any one of the anti-TIGIT antibodies tiragolumab, vibostolimab, domvanalimab, etigilimab, BMS-986207, EOS-448, COM902, ASP8374, SEA-TGT, BGB-A1217, IBI-939 or M6223, and/or any one of the anti-LAG3 antibodies relatlimab, ieramilimab, encelimab, tebotelimab, REGN3767, FS118, IMP701, or IMP731. The present invention thus relates to a compound of formula (I) or a pharmaceutically acceptable salt or solvates thereof, or a pharmaceutical composition comprising any of the aforementioned entities optionally in combination with a pharmaceutically acceptable excipient, for use in the treatment or prevention of cancer, wherein the compound or the pharmaceutical composition is to be administered in combination with one or more immune checkpoint inhibitors, wherein said one or more immune checkpoint inhibitors are preferably selected from anti-CTLA-4 antibodies, anti-PD-1 antibodies, anti-PD-L1 antibodies, anti-TIGIT antibodies, and/or anti-LAG3 antibodies (for example, said one or more immune checkpoint inhibitors may be selected from anti-CTLA-4 antibodies, anti-PD-1 antibodies and/or anti-PD-L1 antibodies, such as, e.g., ipilimumab, tremelimumab, nivolumab, pembrolizumab, cemiplimab, spartalizumab, camrelizumab, sintilimab, tislelizumab, AMP-224, AMP-514, atezolizumab, avelumab, durvalumab, KN035, or CK-301); more preferably, said one or more immune checkpoint inhibitors are selected from ipilimumab, tremelimumab, nivolumab, pembrolizumab, pidilizumab, cemiplimab, dostarlimab, spartalizumab, camrelizumab, sintilimab, tislelizumab, toripalimab, zimberelimab, AMP-224, AMP-514, JTX-4014, INCMGA00012, APE02058, atezolizumab, avelumab, durvalumab, KN035, CK-301, BMS-936559, MEDI4736, MPDL3280A, MDX-1105, MEDI6469, bintrafusp alfa, tiragolumab, vibostolimab, domvanalimab, etigilimab, BMS-986207, EOS-448, COM902, ASP8374, SEA-TGT, BGB-A1217, IBI-939, M6223, relatlimab, ieramilimab, encelimab, tebotelimab, REGN3767, FS118, IMP701, and IMP731.

Moreover, a compound of formula (I) or a pharmaceutically acceptable salt or solvate thereof, or a pharmaceutical composition comprising any of the aforementioned entities, may also be administered in combination with an anti-CCR8 antibody (particularly an antagonistic anti-CCR8 antibody), such as, e.g., DT-7012, BMS-986340, S-531011, BAY-3375968, GS-1811 (or JTX-1811), FPA157, SRF114, HBM1022, or LM-108. Accordingly, the present invention relates to a compound of formula (I) or a pharmaceutically acceptable salt or solvates thereof, or a pharmaceutical composition comprising any of the aforementioned entities optionally in combination with a pharmaceutically acceptable excipient, for use in the treatment or prevention of cancer, wherein the compound or the pharmaceutical composition is to be administered in combination with one or more anti-CCR8 antibodies (which may be selected, e.g., from DT-7012, BMS-986340, S-531011, BAY-3375968, GS-1811 (or JTX-1811), FPA157, SRF114, HBM1022, and LM-108).

The present invention thus particularly relates to a compound of formula (I) or a pharmaceutically acceptable salt or solvate thereof, or a pharmaceutical composition comprising any of the aforementioned entities optionally in combination with a pharmaceutically acceptable excipient, for use in the treatment or prevention of cancer, wherein the compound or the pharmaceutical composition is to be administered in combination with one or more anticancer drugs (including any one or more of the specific anticancer drugs described herein above).

The combinations referred to above may conveniently be presented for use in the form of a pharmaceutical formulation. The individual components of such combinations may be administered either sequentially or simultaneously/concomitantly in separate or combined pharmaceutical formulations by any convenient route. When administration is sequential, either the compound of the present invention (i.e., the compound of formula (I) or a pharmaceutically acceptable salt or solvate thereof) or the further therapeutic agent(s) may be administered first. When administration is simultaneous, the combination may be administered either in the same pharmaceutical composition or in different pharmaceutical compositions. When combined in the same formulation, it will be appreciated that the two or more compounds must be stable and compatible with each other and the other components of the formulation. When formulated separately, they may be provided in any convenient formulation and may be administered by any convenient route. For the combinations described above, it is preferred that the individual components of such combinations are provided in separate pharmaceutical formulations.

The subject or patient to be treated in accordance with the present invention may be an animal (e.g., a non-human animal). Preferably, the subject/patient is a mammal. More preferably, the subject/patient is a human (e.g., a male human or a female human) or a non-human mammal (such as, e.g., a guinea pig, a hamster, a rat, a mouse, a rabbit, a dog, a cat, a horse, a monkey, an ape, a marmoset, a baboon, a gorilla, a chimpanzee, an orangutan, a gibbon, a sheep, cattle, or a pig). Most preferably, the subject/patient to be treated in accordance with the invention is a human.

The term “treatment” of a disorder or disease, as used herein, is well known in the art. “Treatment” of a disorder or disease implies that a disorder or disease is suspected or has been diagnosed in a patient/subject. A patient/subject suspected of suffering from a disorder or disease typically shows specific clinical and/or pathological symptoms which a skilled person can easily attribute to a specific pathological condition (i.e., diagnose a disorder or disease).

The “treatment” of a disorder or disease may, for example, lead to a halt in the progression of the disorder or disease (e.g., no deterioration of symptoms) or a delay in the progression of the disorder or disease (in case the halt in progression is of a transient nature only). The “treatment” of a disorder or disease may also lead to a partial response (e.g., amelioration of symptoms) or complete response (e.g., disappearance of symptoms) of the subject/patient suffering from the disorder or disease. Accordingly, the “treatment” of a disorder or disease may also refer to an amelioration of the disorder or disease, which may, e.g., lead to a halt in the progression of the disorder or disease or a delay in the progression of the disorder or disease. Such a partial or complete response may be followed by a relapse. It is to be understood that a subject/patient may experience a broad range of responses to a treatment (such as the exemplary responses as described herein above). The treatment of a disorder or disease may, inter alia, comprise curative treatment (preferably leading to a complete response and eventually to healing of the disorder or disease) and palliative treatment (including symptomatic relief).

The term “prevention” of a disorder or disease, as used herein, is also well known in the art. For example, a patient/subject suspected of being prone to suffer from a disorder or disease may particularly benefit from a prevention of the disorder or disease. The subject/patient may have a susceptibility or predisposition for a disorder or disease, including but not limited to hereditary predisposition. Such a predisposition can be determined by standard methods or assays, using, e.g., genetic markers or phenotypic indicators. It is to be understood that a disorder or disease to be prevented in accordance with the present invention has not been diagnosed or cannot be diagnosed in the patient/subject (for example, the patient/subject does not show any clinical or pathological symptoms). Thus, the term “prevention” comprises the use of a compound of the present invention before any clinical and/or pathological symptoms are diagnosed or determined or can be diagnosed or determined by the attending physician.

It is to be understood that the present invention specifically relates to each and every combination of features and embodiments described herein, including any combination of general and/or preferred features/embodiments. In particular, the invention specifically relates to each combination of meanings (including general and/or preferred meanings) for the various groups and variables comprised in formula (I).

In this specification, a number of documents including patent applications and scientific literature are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The reference in this specification to any prior publication (or information derived therefrom) is not and should not be taken as an acknowledgment or admission or any form of suggestion that the corresponding prior publication (or the information derived therefrom) forms part of the common general knowledge in the technical field to which the present specification relates.

The invention will now be described by reference to the following examples which are merely illustrative and are not to be construed as a limitation of the scope of the present invention.

The compounds of formula (I) described in the following examples section are defined by their chemical formulae and their corresponding chemical names. In case of conflict between any chemical formula and the corresponding chemical name indicated herein, the present invention relates to both the compound defined by the chemical formula and the compound defined by the chemical name, and particularly relates to the compound defined by the chemical formula.

### EXAMPLES

### General Experimental Procedures

### General Conditions

All reagents were commercial grade and used without further purification. Reactions were typically run using commercial anhydrous solvents under argon atmosphere.

Column chromatography was generally performed with a Biotage Isolera® Four apparatus using, unless stated otherwise, Biotage® KP-Sil cartridge pre-filled with 50 μm irregular silica gel. Alternatively, Interchim® PURIFLASH jumbo pack silica HP cartridges pre-filed with 50 μm silica gel (mentioned as Interchim® 50 μm), Interchim® PURIFLASH jumbo pack silica SDT cartridges pre-filed with 20 μm silica gel (mentioned as Interchim® 20 μm), or Biotage Sfar® KP-Amino D cartridges pre-filed with 50 μm silica gel (mentioned as Biotage® KPNH) could be used when necessary. Alternatively, column chromatography was performed with a Biotage Isolera Spektra One® using Silica gel 60® (40-63 μm, Merck) (mentioned as Merck 60®).

Thin layer chromatography (TLC) was carried out using pre-coated silica gel F-254 plates or Biotage KP—NH TLC plates.

Releasing of free bases from the corresponding salts was carried out using Biotage ISOLUTE® SCX-2 cation exchange cartridges.

¹H-NMR spectra were recorded on a Bruker AV-300 spectrometer or on a Bruker AMX-400 spectrometer. Proton chemical shifts are listed relative to residual CD₃OD (3.31 ppm), DMSO (2.50 ppm), D₂O (4.78 ppm) or CDCl₃ (7.26 ppm). Splitting patterns are designated as s (singlet), d (doublet), dd (doublet-doublet), t (triplet), tt (triplet-triplet), td (triplet-doublet), q (quartet), quint (quintuplet), sex (sextuplet), sept (septuplet), m (multiplet), bs (broad).

UPLC-MS analyses were recorded with an UPLC Waters Aquity platform with a photodiode array detector (210-400 nm) using an Acquity CSH C₁₈ 1.7 μm (2.1×30 mm) column. The mobile phase consisted in a gradient of water with 0.025% of trifluoroacetic acid (TFA) and acetonitrile with 0.025% of TFA. The flow rate was 0.8 mL per min. All analyses were performed at 55° C. The UPLC system was coupled with a Waters SQD2 platform. All mass spectra were full-scan experiments (mass range 100-800 amu) and were obtained using electrospray ionization.

HPLC-MS were recorded using an HPLC Waters platform with a 2767 sample manager, a 2525 pump, a photodiode array detector (200-400 nm). This HPLC system was coupled with a Waters Acquity QDa detector. Mass spectra were full-scan experiments (mass range 110-850 amu) and were obtained using electro spray ionization. For analytical samples, the selected column was a XSelect CSH C₁₈ 3.5 μm (2.1×30 mm) column. For preparative purifications, the selected column was, unless otherwise stated, an XSelect CSH prep C₁₈ 5 μm (19×100 mm) column. The mobile phase consisted in an appropriate gradient of water with 0.1% of formic acid and acetonitrile with 0.1% of formic acid. The flow rate was 1 mL/min in analytical mode, and in preparative mode 25 mL/min.

Alternatively, HPLC-MS were recorded using a Thermo LC/MS-Ultimate 3000-Ion Trap HCT Brucker. Mass spectra were performed on a Brucker Ion Trap and were obtained using electrospray ionization. For analytical samples, the selected column was a Nucleodur 3 μm 4.6×100 mm reverse-phase column. The mobile phase consisted in a linear gradient with a flow rate of 1.3 mL/min from 95% A and 5% B to 5% A and 95% B in 8.5 min (solvent A, H₂O with 0.1% formic acid; solvent B, acetonitrile with 0.1% formic acid). Preparative purifications were performed on a Gilson PLC 2020 apparatus using a column C8 Princeton SPHER.60-10 μm, mentioned as Column B. The mobile phase consisted in a gradient of acetonitrile (5 to 100%) in water+0.1% formic acid with a flow rate of 30 mL/min.

After preparative HPLC, the fractions were combined, eventually partially concentrated under reduced pressure, then freeze dried from a water acetonitrile mixture.

All HPLC-MS were performed at room temperature.

Melting points were measured on a Barnstead Electrothermal 9100 or an Electrothermal 1002 and are not corrected.

Unless mentioned otherwise all compounds isolated by filtration or centrifugation were dried overnight in high vacuum at 50-70° C.

### General Procedures

### General Procedure (I): Nucleophilic Aromatic Substitution

To a solution of haloheteroaryl (1.0 eq.) in toluene (0.15 M) at 0° C., was added alkylnitrile (1.0 eq.) and sodium hexamethyldisilazide (NaHMDS) 2M in tetrahydrofuran (THF) (1.0 eq.). The reaction mixture was stirred at 0° C. for 2 hours, then hydrolysed with ice and NH₄Cl (sat. aq.) and extracted with ethyl acetate (EtOAc). The organic layer was washed with brine, dried over magnesium sulfate then concentrated to dryness to obtain the corresponding heteroaryl acetonitrile.

### General Procedure (Ib): Nucleophilic Aromatic Substitution

To a solution of haloheteroaryl (1.0 eq.) in toluene (0.45 M, 1V) at 0° C., was added ester (1.0 eq.) then Lithium hexamethyldisilazide (LiHMDS) 1M in tetrahydrofuran (THF) (2.0 eq.) was added dropwise. The reaction mixture was stirred at 25° C. for 24 hours, then hydrolysed with NH₄Cl (sat. aq., 4V), diluted with water (2V) and extracted with ethyl acetate (EtOAc 2*4V). The organic layer was washed with brine, dried over magnesium sulfate then concentrated to dryness to obtain the corresponding heteroaryl ester.

### General Procedure (IIa): Nitrile Hydration

To a solution of nitrile (1.0 eq.) in dimethyl sulfoxide (DMSO) (0.2 M) was added potassium carbonate (2.0-4.0 eq.), and hydrogen peroxide aqueous 30% (3.0-10 eq.). The reaction was stirred at 25° C. for 1 to 16 hours. The reaction mixture was diluted with water then extracted with EtOAc. The organic layer was washed with brine, dried over magnesium sulfate, and concentrated to dryness. The crude was purified by flash chromatography to afford the corresponding amide.

### General Procedure (IIb): Nitrile Hydration

To a solution of nitrile (1.0 eq.) in DMSO (0.2 M, 1 V) was added potassium carbonate (2.0-4.0 eq.), and hydrogen peroxide aqueous 30% (3.0-10 eq.). The reaction was stirred at 25° C. for 1 to 16 hours. The reaction mixture was diluted with water (50 V), the obtained precipitate was collected by filtration, and washed with water (20 V) to afford the corresponding amide.

### General Procedure (III): Cyclisation

To a solution of amide (1.0 eq.) in t-BuOH (0.1 M) were added NaOtBu (3.0 eq.) and benzene-1,2-diamine (0.2 eq.). The mixture was sparged with Argon for 10 min before addition of CuO (0.1 equiv). The reaction mixture was heated at 100° C. for 18 hours. The reaction mixture was filtered through a pad of Celite, the pad was washed with EtOAc and dichloromethane (DCM) then the filtrate was concentrated to dryness. The crude residue was purified by flash chromatography to afford the corresponding dihydro-pyrroloheteroaryl.

### General Procedure (IV): Aryl Cyanation

Under inert atmosphere, to a solution of aryl halide (1.0 eq.) in DMA (0.3M) was added Zinc(II) cyanide (1.5 eq.). The mixture was sparged with argon for 10 min before addition of Bis(tri-tert-butylphosphine)palladium (0.1 eq.). The reaction mixture was heated at 130° C. for 18 hours. The reaction mixture was filtered through a pad of Celite and washed with EtOAc and DCM. then the filtrate was concentrated to dryness. The residue was dissolved in EtOAc, washed with NaHCO₃ sat. aq., brine, dried over magnesium sulfate and concentrated to dryness. The crude was purified by flash chromatography to afford the corresponding nitrile.

### General Procedure (V): Ullmann Coupling

Under inert atmosphere, to a solution of amide (1.0 eq.) in dioxane (0.1 M) were added potassium carbonate (3.0 eq.), (1R,2S)—N1,N2-dimethylcyclohexane-1,2-diamine (0.2 eq.), and aryl halide (1.1 eq.). The mixture was sparged with argon for 10 min before addition of CuI (0.1 eq.). The reaction mixture was heated at 100° C. for 18 hours. The reaction mixture was filtered through a pad of Celite and washed with EtOAc and DCM, then the filtrate was concentrated to dryness. The crude was purified by flash chromatography to afford the corresponding N-arylamide.

General Procedure (VI): Formation of Methyl Ester from Amide

Under inert atmosphere, to a solution of amide (1.0 eq.) in MeOH (0.1 M) was added 1,1-dimethoxy-N,N-dimethylmethanamine (6 eq.). The reaction mixture was heated at 60° C. for 16 to 24 hours. The reaction mixture was concentrated to dryness. The crude was purified by flash chromatography to afford the corresponding methyl ester.

### General Procedure (VII): Ester Hydrolysis

To a solution of methyl ester (1.0 equiv) in THE (0.1 M) was added lithium hydroxide 1M aq. (1.5 equiv). The reaction was stirred at 25° C. for 1 to 18 hours. The reaction mixture was acidified with HCl 1M aq. to pH=1 then extracted with EtOAc. The organic layer was dried over magnesium sulfate and concentrated to dryness to afford the corresponding acid.

### General Procedure (VIIIa): Peptidic Coupling

To a mixture of acid in DCM (0.1 M) or in DCM/THF (1:1, 0.1 M) or in Propylene carbonate (0.1 M) was added ((1H-benzo[d][1,2,3]triazol-1-yl)oxy)tris(dimethylamino)phosphonium hexafluorophosphate(V) (1.3 eq.), N,N-diisopropylethylamine (1.0 to 5.0 eq) and the amine (1.0 to 2.0 eq.). The reaction mixture was stirred at 25° C. for 3 to 18 hours. The reaction mixture was hydrolysed with NH₄Cl sat. aq. then extracted with EtOAc. The organic layer was washed with brine, dried over magnesium sulfate then concentrated to dryness to afford the corresponding crude amide.

### General Procedure (VIIIb): Peptidic Coupling

To a mixture of acid in THE (0.1 M) or in MeTHF (0.1 M) was added N-[1-(Cyano-2-ethoxy-2-oxoethylideneaminooxy)dimethylamino(morpholino)uronium Hexafluorophosphate (1.2 eq.), triethylamine (1.0 to 5.0 eq) and the amine (1.0 to 2.0 eq.). The reaction mixture was stirred at 25° C. for 3 to 18 hours. The reaction mixture was hydrolysed with NH₄Cl sat. aq. then extracted with EtOAc. The organic layer was washed with brine, dried over magnesium sulfate then concentrated to dryness to afford the corresponding crude amide.

### General Procedure (IX): Amide Reduction

To a solution of amide in THE (0.1 M) at 0° C. were added sodium borohydride (5.0 eq.) and dropwise boron trifluoride diethyl etherate (7.0 eq.). The reaction mixture was stirred at 25° C. for 5 hours. At 0° C., the reaction mixture was hydrolysed with cooled water, stirred at 25° C. for 18 hours then extracted with EtOAc. The organic layer was washed with brine, dried over magnesium sulfate, and concentrated to dryness. The crude was purified by flash chromatography to afford the corresponding amine.

### General Procedure (X): Buchwald Coupling

Under inert atmosphere, to a solution of amine (1.0-2.0 equiv) and halide or pseudo halide (1.0-2.0 equiv) in toluene (0.1-0.2 M) was added sodium tert-butoxide (1.5 equiv). The mixture was sparged with Argon for 10 min before addition of RuPhos PdG4 (0.05 equiv). The reaction mixture was heated at 80° C. for 16 hours. The reaction mixture was filtered through a pad of Celite, the pad was washed with EtOAc then the filtrate was concentrated to dryness.

The crude residue was purified by flash chromatography to afford the corresponding N-Aryl amine.

### General Procedure (Xb): Buchwald Coupling

Under inert atmosphere, to a solution of amine (1.0-2.0 equiv) and halide or pseudo halide (1.0-2.0 equiv) in Dioxane (0.1 M) was added Cesium Carbonate (3 equiv). The mixture was sparged with Argon for 10 min before addition of RuPhos PdG4 (0.10 equiv). The reaction mixture was heated at 100° C. for 16 hours. The reaction mixture was filtered through a pad of Celite, the pad was washed with EtOAc then the filtrate was concentrated to dryness. The crude residue was purified by flash chromatography to afford the corresponding N-Aryl amine.

### General Procedure (Xc): Buchwald Coupling

Under inert atmosphere, to a solution of amine (1.0 equiv) in Dioxane (0.1 M) was added Cesium Carbonate (3 equiv). The mixture was sparged with Argon for 10 min before addition of RuPhos PdG4 (0.10 equiv). The reaction mixture was heated at 100° C. for 4 hours, then halide or pseudo halide (2.0 equiv) was added and the reaction mixture was further heated for 20 hours. The reaction mixture was filtered through a pad of Celite, the pad was washed with EtOAc then the filtrate was concentrated to dryness. The crude residue was purified by flash chromatography to afford the corresponding N-Aryl aza-indoline.

### General Procedure (Xd): Buchwald Coupling

Under inert atmosphere, to a solution of precursor (1.0 equiv) in Dioxane (0.1 M) was added Cesium Carbonate (3 equiv). The mixture was sparged with Argon for 10 min before addition of SPhos PdG4 (0.10 equiv). The reaction mixture was heated at 100° C. for 20 hours. The reaction mixture was filtered through a pad of Celite, the pad was washed with EtOAc then the filtrate was concentrated to dryness. The crude residue was purified by flash chromatography to afford the corresponding cyclised product.

### General Procedure (XI): Methyl Ester Cleavage

To a solution of methyl ester in ^{t}BuOH (0.05 M) was added Sodium ^{t}Butoxide (10 eq). The reaction was stirred at 60° C. for 18 hours. The reaction mixture was diluted with water and acidified with HCl aq. 1M to pH=3 then extracted with EtOAc. The organic layer was dried over magnesium sulfate and concentrated to dryness to afford the corresponding acid.

### General Procedure (XII): Amine Boc Deprotection

A solution of boc protected amine in DCM (0.2 M, 1 V) or in dioxane (0.2 M, 1 V) was treated with TFA (1 V) or HCl (2M in Et₂O, 1 V) or HCl (4N in dioxane, 0.5 V). The reaction mixture was stirred at 25° C. for 1 hour then concentrated under reduced pressure to obtain the corresponding crude amine.

### General Procedure (XIII): Nucleophilic Displacement

To a solution of nucleophile (alcohol, amide) in THE or in DMF or in DMA or in NMP (0.2 M, 1 V) was added NaH (sodium hydride 60% in mineral oil) (1.0-5.0 eq) at 0° C. The mixture was stirred at 0° C. for 20 minutes then the electrophile was added (1.0-3.0 eq.). The reaction mixture was stirred at 25° C. for 18 hours then quenched in NH₄Cl (sat.aq. 10 V) extracted with EtOAc. The organic layer was dried over magnesium sulfate and concentrated to dryness to afford the corresponding substituted product.

### General Procedure (XIV): Aminocarbonylation

In the first chamber of a two-chamber reactor (2*20 mL) was prepared a solution of halide or pseudo-halide, amine (1.0-2.0 eq), and triethyl amine (3.0-5.0 eq) in dioxane (0.12 M, 1 V). In the second chamber a suspension of Mo(CO)₆ (1.0-2.5 eq) in dioxane (1V) was prepared. Both chambers were sparged with argon for 10 minutes then XantPhos Pd G4 (0.10 eq) was added in the first chamber, and DBU (2.0-5.0 eq) was added in the second chamber. The set up was crimped and heated at 110° C. for 16 hours. The mixture in the first chamber was filtered over a Celite pad, rinsed with DCM (3 V), the filtrate was washed with NH₄Cl (3 V, sat.aq.), brine (3 V) then dried over magnesium sulfate and concentrated under reduced pressure to obtain crude aminocarbonylated product.

### General Procedure (XVa): Nitrile Reduction

To a solution of nitrile in MeOH (0.1 M, 1 V) was added Cobalt^{(II)} chloride hexahydrate (1 equiv.) and di-tert-butyl dicarbonate (2 equiv.), the solution was cooled at −78° C. for 15 minutes, then Sodium borohydride (7 equiv.) was added. The reaction mixture was let warm up at −20° C. for 4 hours, then at 25° C. for 30 minutes. The reaction mixture was quenched with NH₄Cl (1 V, sat. aq.), diluted with water (1V) and extracted with EtOAc (2*1 V). Combined organic layers were washed with brine, dried over MgSO₄, and concentrated under reduced pressure. The crude was dissolved in DCM (0.25 M) and treated with trifluoroacetic acid (10 equiv.) at 25° C. for 16 hours. The reaction mixture was concentrated under reduced pressure, then purified by Ion Exchange chromatography (Isolute SCX-2, load in DCM, elution with NH₃ 1N in MeOH). The eluate was concentrated under reduced pressure to obtain the corresponding amine.

### General Procedure (XVb): Nitrile Reduction

To a solution of nitrile in EtOH (0.15 M, 1 V) was added ammonium hydroxide (20 equiv.). The reaction mixture was sparged with Argon for 10 min then Raney Nickel (type 2800, 1 equiv.) was added and the solution was sparged with Hydrogen for 5 minutes then the reaction mixture was heated at 50° C. under a hydrogen atmosphere (1 Bar) for 18 hours. The reaction mixture was sparged with Argon for 10 minutes then treated with Sodium nitrate (10% aq. 1 V) and the mixture was stirred at 25° C. for 1 hour. The mixture was filtered over a Celite pad which was rinsed with EtOH (2 V). The filtrate was partially concentrated under reduced pressure, treated with NaOH (1N, 0.5 V) and extracted with EtOAc (3*1 V). The combined organic layers were washed with brine (1 V), dried over MgSO₄, and concentrated under reduced pressure to obtain the corresponding amine.

### General Procedure (XVI): Aromatic Bromination

To a solution of aromatic in MeCN or EtOAc (0.1 M, 1 V) was added N-bromosuccinimide (1.0 equiv.) followed by acetic acid (1.0 equiv.). The reaction mixture was stirred at 25° C. for 1 hour, then washed with K₂CO₃ (sat. aq., 1 V), with Brine (0.3 V), dried over MgSO₄, and concentrated under reduced pressure to obtain the crude corresponding brominated aromatic compound.

### General Procedure (XVII): N-Aryl N-Allyl Amino-Pyrazine Synthesis

To a solution of 2,3-dichloropyrazine and aniline (1.0 equiv) in MeTHF (0.2 M, 1 V) was added Potassium tert-butoxide (3 equiv.). The reaction mixture was stirred at 25° C. for 2 hours then 3-bromo-2-methylprop-1-ene was added (1.2 equiv.) and the reaction mixture was heated at 40° C. for 18 hours. The reaction mixture was quenched with NH₄Cl (sat.aq. 0.3 V), diluted with water (0.2 V) and extracted with EtOAc (2*0.3 V). The combined organic layers were washed with brine (0.3 V) dried over MgSO₄ then concentrated under reduced pressure to afford the crude corresponding N-aryl N-allyl amino-pyrazine.

### General Procedure (XVIII): Pseudo-Heck Cyclisation

To a solution of N-aryl N-allyl amino-aryl in a DMSO/water mixture (95:5, 0.5 M, 1 V) was added Sodium formate (1.2 equiv.), the reaction mixture was sparged with Argon for 5 minutes then Palladium^{(II)} acetate (0.05 equiv.) was added and the mixture was heated at 100° C. for 30 minutes. The reaction mixture was filtered over a celite pad, which was rinsed with water (3 V) then with EtOAc (1 V) and the filtrate was extracted with EtOAc (2*1 V). The combined organic layers were washed with brine (1 V) dried over MgSO₄ then concentrated under reduced pressure to afford the crude corresponding cyclised product.

### General Procedure (XIX): Methoxycarbonylation

In the first chamber of a two-chamber reactor (2*20 mL) was prepared a solution of halide or pseudo-halide, and triethyl amine (2.0 eq) in a dioxane/methanol mixture (1:1 0.15 M, 1 V). In the second chamber a suspension of Mo(CO)₆ (0.5 eq) in dioxane (1V) was prepared. Both chambers were sparged with argon for 10 minutes then XantPhos Pd G4 (0.10 eq) was added in the first chamber, and DBU (1.5 eq) was added in the second chamber. The set up was crimped and heated at 80° C. for 16 hours. The mixture in the first chamber was filtered over a Celite pad and rinsed with DCM (3 V). The filtrate was concentrated under reduced pressure to obtain the crude corresponding methoxycarbonylated product.

### General Procedure (XX): Ester Reduction

To a solution of ester in THF (0.2 M, 1 V) at 0° C. was added dropwise Diisobutylaluminium hydride (1M in THF, 6 equiv.). The reaction mixture was stirred at 25° C. for 4 hours, then quenched with NaOH (4N aq. 2 V) at 0° C. The mixture was stirred for another 20 minutes then extracted with EtOAc (3*1 V). The combined organic layers were washed with brine, dried over MgSO₄, and concentrated under reduced pressure to obtain the crude corresponding alcohol.

### General Procedure (XXI): Alcohol Oxidation

To a solution of alcohol in DCM (0.1 M, 1 V) at 0° C. was added portionwise Dess-martin Periodinane (1.5 equiv.). The reaction mixture was stirred at 25° C. for 1 hour, then quenched with Na₂S₂O₃ (sat. Aq. 1 V) and extracted with DCM (2*1 V). The combined organic layers were washed with brine, dried over MgSO₄, and concentrated under reduced pressure to obtain the crude corresponding aldehyde.

### General Procedure (XXII): Reductive Amination

To a solution of aldehyde in DCM (0.1 M, 1 V) at 25° C. was added the amine (1.5 equiv.) and Sodium triacetoxyhydroborate (1.5 equiv.). The reaction mixture was stirred at 60° C. for 18 hour, then quenched with NaHCO₃ (sat. Aq. 2 V) and extracted with DCM (2*8 V). The combined organic layers were washed with brine, dried over MgSO₄, and concentrated under reduced pressure to obtain the crude corresponding aldehyde.

### General Procedure (XXIII): Intramolecular Nucleophilic Aromatic Substitution

To a solution of amino-haloheteroaryl (1.0 eq.) in Me-THF (0.1 M, 1V) at 0° C., was added Potassium ^{t}butoxide (1.5 equiv.). The reaction mixture was stirred at 25° C. for 1 hour, then hydrolysed with NH₄Cl (sat. aq., 4V), diluted with water (2V) and extracted with ethyl acetate (EtOAc 2*4V). The organic layer was washed with brine, dried over magnesium sulfate then concentrated to dryness to obtain the corresponding cyclised product.

The synthesis and characterization of various exemplary compounds of formula (I) as well as corresponding synthetic intermediates are described in the following (where they are referred to as “Examples” and “Compounds”, respectively).

### Compound 1: ethyl 2-(4-(tert-butoxycarbonyl)-3,3-dimethylpiperazin-1-yl)-4-methylpyrimidine-5-carboxylate

To a solution of ethyl 2-chloro-4-methylpyrimidine-5-carboxylate (300 mg) in MeCN (15 mL) was added Potassium carbonate (1.5 eq.) and tert-butyl 2,2-dimethylpiperazine-1-carboxylate (1.2 eq.). The reaction mixture was heated at 80° C. for 16 hours then diluted with EtOAc (100 mL), washed with water (100 mL), brine (100 mL), the organic layer was dried over MgSO₄ and concentrated to dryness. The crude was purified by flash chromatography (Interchim® 50 μm, CyHex 100% to CyHex/EtOAc 50:50) to obtain Compound 1 (560 mg, 99%) as a white solid.

M/Z (M+H)⁺: 379.3

### Compound 2: ethyl 2-(3,3-dimethylpiperazin-1-yl)-4-methylpyrimidine-5-carboxylate hydrochloride

Compound 2 was prepared according to general procedure (XII) starting from Compound 1 (560 mg) in DCM with TFA. The crude was dissolved in DCM (5 mL), treated with HCl (2N in Et₂O, 5 mL), concentrated under reduced pressure, then dissolved in HCl (1M in MeOH, 5 mL) and concentrated under reduced pressure to obtain Compound 2 (450 mg, 97%) as a white solid.

M/Z (M+H)⁺: 279.3

### Compound 3: ethyl 2-(4-(tert-butoxycarbonyl)-3,3-dimethylpiperazin-1-yl)pyrimidine-5-carboxylate

To a solution of ethyl 2-chloropyrimidine-5-carboxylate (250 mg) in MeCN (15 mL) was added Potassium carbonate (1.5 eq.) and tert-butyl 2,2-dimethylpiperazine-1-carboxylate (1.2 eq.). The reaction mixture was heated at 80° C. for 16 hours then diluted with EtOAc (100 mL), washed with water (100 mL), brine (100 mL), the organic layer was dried over MgSO₄ and concentrated to dryness. The crude was purified by flash chromatography (Interchim®50 μm, CyHex 100% to CyHex/EtOAc 80:20) to obtain Compound 3 (478 mg, 98%) as a white solid.

M/Z (M−^{t}Bu+H)⁺: 309.2

### Compound 4: ethyl 2-(3,3-dimethylpiperazin-1-yl)pyrimidine-5-carboxylate hydrochloride

Compound 4 was prepared according to general procedure (XII) starting from Compound 3 (475 mg) in DCM with TFA. The crude was dissolved in DCM (5 mL), treated with HCl (2N in Et₂O, 5 mL), concentrated under reduced pressure, then dissolved in HCl (1M in MeOH, 5 mL) and concentrated under reduced pressure to obtain Compound 4 (420 mg, quant.) as a white solid.

M/Z (M+H)⁺: 265.3

### Compound 5: ethyl 2-(4-(tert-butoxycarbonyl)-3,3-dimethylpiperazin-1-yl)thiazole-4-carboxylate

Compound 5 was prepared according to general procedure (X) starting from ethyl 2-bromothiazole-4-carboxylate (300 mg) and tert-butyl 2,2-dimethylpiperazine-1-carboxylate (1.2 eq.). The crude was purified by flash chromatography (Interchim® 20 μm, CyHex 100% to CyHex/EtOAc 70:30) to obtain Compound 5 (344 mg, 73%) as a yellow solid.

M/Z (M[+H)⁺: 370.3

### Compound 6: ethyl 2-(3,3-dimethylpiperazin-1-yl)thiazole-4-carboxylate

Compound 6 was prepared according to general procedure (XII) starting from Compound 5 (344 mg) in dioxane with HCl in dioxane at 70° C. for 16 hours. The crude was treated with K₂CO₃ (sat.aq., 50 mL) and extracted with EtOAc (2*50 mL). Combined organic layers were washed with brine (50 mL), dried over MgSO₄ and concentrated under reduced pressure to obtain Compound 6 (217 mg, 87%) as an orange oil.

M/Z (M+H)⁺: 270.2

### Compound 7: methyl 6-((2-((tert-butoxycarbonyl)(methyl)amino)ethyl)(methyl)amino)-2,4-dimethylnicotinate

Compound 7 was prepared according to general procedure (X) starting from methyl 6-chloro-2,4-dimethylnicotinate (250 mg) and tert-butyl methyl(2-(methylamino)ethyl)carbamate (1.1 eq.). The crude was purified by flash chromatography (Merck 60®, CyHex 100% to CyHex/EtOAc 50:50) to obtain Compound 7 (328 mg, 75%) as a white solid.

M/Z (M[+H)⁺: 352.2

### Compound 8: methyl 2,4-dimethyl-6-(methyl(2-(methylamino)ethyl)amino)nicotinate hydrochloride

Compound 8 was prepared according to general procedure (XII) starting from Compound 7 (328 mg) in dioxane with HCl in dioxane for 4 hours. The crude was triturated in Et₂O (20 mL) and further washed with Et₂O (3*15 mL) to obtain Compound 8 (254 mg, 95%) as a beige solid.

M/Z (M+H)⁺: 252.1

### Compound 9: tert-butyl 4-(5-(methoxycarbonyl)-4,6-dimethylpyridin-2-yl)-2,2-dimethylpiperazine-1-carboxylate

Compound 9 was prepared according to general procedure (X) starting from methyl 6-chloro-2,4-dimethylnicotinate (1.80 g) and tert-butyl 2,2-dimethylpiperazine-1-carboxylate (1.1 eq.). The crude was purified by flash chromatography (Merck 60®, CyHex 100% to CyHex/EtOAc 80:20) to obtain Compound 9 (3.27 g, 96%) as a white solid.

M/Z (M[+H)⁺: 378.3

### Compound 10: methyl 6-(3,3-dimethylpiperazin-1-yl)-2,4-dimethylnicotinate hydrochloride

Compound 10 was prepared according to general procedure (XII) starting from Compound 9 (3.40 g) in dioxane with HCl in dioxane for 4 hours. The crude was triturated in a mixture of Et₂O (100 mL) and EtOH (3 mL) and further washed with Et₂O (3*15 mL) to obtain Compound 10 (2.80 g, 99%) as a white solid.

M/Z (M+H)⁺: 278.2

### Compound 11: 6-(3,3-dimethylpiperazin-1-yl)-2,4-dimethylnicotinic acid

Compound 11 was prepared according to general procedure (XI) starting from Compound 10 (100 mg). The reaction mixture was concentrated under reduced pressure, the residue was treated with HCl (4N in dioxane, 20 mL), and the mixture was concentrated under reduced pressure. The crude was dissolved in MeOH and the methanolic solution was charged on an Isolute SCX-2 cartridge (2 g), the cartridge was rinsed with MeOH then eluted with triethylamine (1M in methanol) to obtain Compound 11 (53 mg, 64%) as a white solid.

M/Z (M+H)⁺: 264.2

### Compound 12: tert-butyl (1-carbamoylcyclopropyl)(methyl)carbamate

To a solution of 1-((tert-butoxycarbonyl)(methyl)amino)cyclopropane-1-carboxylic acid (250 mg) in THE (15 mL) was added N,N-diisopropylethylamine (3.0 eq.), then at 0° C. Methyl chlorocarbonate (1.5 eq.) was added dropwise. The reaction mixture was stirred at 0° C. for 30 minutes, ammonia was added (0.4 M in dioxane, 3.0 eq.) and the mixture was let warm up at 25° C. over 18 hours, then concentrated under reduced pressure. The residue was treated with NaHCO₃ (sat. aq., 50 mL) and extracted with EtOAc (3*100 mL). Combined organic layers were dried over MgSO₄ and concentrated under reduced pressure to obtain Compound 12 (238 mg, 96%) as a white solid.

M/Z (M+Na)⁺: 237.1

### Compound 13: 1-(methylamino)cyclopropane-1-carboxamide hydrochloride

Compound 13 was prepared according to general procedure (XII) starting from Compound 12 (258 mg) in dioxane with HCl in dioxane for 18 hours at 60° C. The crude was triturated in Et₂O (20 mL) to obtain Compound 13 (150 mg, 90%) as a beige solid.

M/Z (M+H)⁺: 115.0

### Compound 14: 4-(3-bromo-6-chloropyridin-2-yl)tetrahydro-2H-pyran-4-carbonitrile

Compound 14 was prepared according to general procedure (I) starting from 3-bromo-6-chloro-2-fluoropyridine (1.50 g) and tetrahydro-2H-pyran-4-carbonitrile (759 μL). The crude was purified by flash chromatography (Merck 60®, CyHex 100% to CyHex/EtOAc 60:40) to obtain Compound 14 (1.67 g, 83%) as a white solid.

M/Z (M[³⁵Cl][⁸¹Br]+H)⁺: 303.0

### Compound 15: 4-(3-bromo-6-chloropyridin-2-yl)tetrahydro-2H-pyran-4-carboxamide

Compound 15 was prepared according to general procedure (IIa) starting from Compound 14 (500 mg). The crude was purified by flash chromatography (Merck 60®, CyHex 100% to CyHex/EtOAc 20:80) to obtain Compound 15 (477 mg, 90%) as a white solid.

M/Z (M[³⁵Cl⁸¹Br]+H)⁺:321.0

### Compound 16: 5′-chloro-2,3,5,6-tetrahydrospiro[pyran-4,3′-pyrrolo[3,2-b]pyridin]-2′(1′H)-one

Compound 16 was prepared according to general procedure (III) starting from Compound 15 (477 mg). The crude was purified by flash chromatography (Merck 60®, CyHex 100% to CyHex/EtOAc 50:50) to obtain Compound 16 (330 mg, 93%) as a brown solid.

M/Z (M[³⁵Cl]+H)⁺:239.0

### Compound 17: 2′-oxo-1′,2,2′,3,5,6-hexahydrospiro[pyran-4,3′-pyrrolo[3,2-b]pyridine]-5′-carbonitrile

Compound 17 was prepared according to general procedure (IV) starting from Compound 16 (330 mg). The crude was purified by flash chromatography (Merck 60®, CyHex 100% to CyHex/EtOAc 50:50) to obtain Compound 17 (263 mg, 83%) as a brown solid.

M/Z (M+H)⁺: 230.1.

### Compound 18: 1′-(4-chloro-3-fluorophenyl)-2′-oxo-1′,2,2′,3,5,6-hexahydrospiro[pyran-4,3′-pyrrolo[3,2-b]pyridine]-5′-carbonitrile

Compound 18 was prepared according to general procedure (V) starting from Compound 17 (260 mg) and 4-bromo-1-chloro-2-fluorobenzene (261 mg, 1.1 eq.). The crude was purified by flash chromatography (Merck 60®, CyHex 100% to CyHex/EtOAc 50:50) to obtain Compound 17 (239 mg, 59%) as a purple solid.

M/Z (M[³⁵Cl]+H)⁺:358.1.

### Compound 19: 1′-(4-chloro-3-fluorophenyl)-2′-oxo-1′,2,2′,3,5,6-hexahydrospiro[pyran-4,3′-pyrrolo[3,2-b]pyridine]-5′-carboxamide

Compound 19 was prepared according to general procedure (IIa) starting from Compound 18 (239 mg). The crude was purified by flash chromatography (Merck 60®, CyHex 100% to CyHex/EtOAc 0:100) to obtain Compound 19 (203 mg, 81%) as a purple solid.

M/Z (M[³⁵Cl]+H)⁺: 376.2

### Compound 20: methyl 1′-(4-chloro-3-fluorophenyl)-2′-oxo-1′,2,2′,3,5,6-hexahydrospiro[pyran-4,3′-pyrrolo[3,2-b]pyridine]-5′-carboxylate

Compound 20 was prepared according to general procedure (VI) starting from Compound 19 (203 mg). The crude was purified by flash chromatography (Merck 60®, CyHex 100% to CyHex/EtOAc 50:50) to obtain Compound 20 (205 mg, 97%) as a white solid.

M/Z (M [³⁵Cl]+H)⁺: 391.2.

### Compound 21: 1′-(4-chloro-3-fluorophenyl)-2′-oxo-1′,2,2′,3,5,6-hexahydrospiro[pyran-4,3′-pyrrolo[3,2-b]pyridine]-5′-carboxylic acid and 6-(4-carboxytetrahydro-2H-pyran-4-yl)-5-((4-chloro-3-fluorophenyl)amino)picolinic acid mixture

Compound 20 (205 mg) was hydrolysed according to general procedure (VII) to obtain Compound 21 (204 mg) as a white solid.

M/Z (M [³⁵Cl]+H)⁺: 377.2

M/Z (M [³⁵Cl]+H)⁺: 395.1

### Example 1: 1′-(4-chloro-3-fluorophenyl)-5′-(2,2-dimethyl-3-oxopiperazine-1-carbonyl)-2,3,5,6-tetrahydrospiro[pyran-4,3′-pyrrolo[3,2-b]pyridin]-2′(1′H)-one

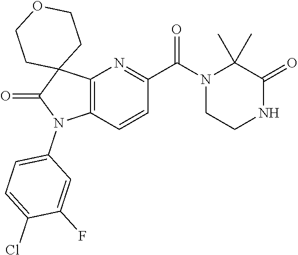 [SMILES: CC1(C)C(=O)NCCN1C(=O)c1ccc2c(n1)C1(CCOCC1)C(=O)N2c1ccc(Cl)c(F)c1]

To a solution of Compound 21 (204 mg) in DCM/THF (1:1, 30 mL) was added ((1H-benzo[d][1,2,3]triazol-1-yl)oxy)tris(dimethylamino)phosphonium hexafluorophosphate(V) (2.3 eq.), N,N-diisopropylethylamine (3.8 eq) and 3,3-dimethylpiperazin-2-one (1.5 eq.). The reaction mixture was stirred at 25° C. for 18 hours. The reaction mixture was hydrolysed then extracted with EtOAc. The organic layer was dried over MgSO₄. The crude was purified by flash chromatography (Merck 60®, CyHex 100% to EtOAc 100%) then further purified by preparative HPLC (Column B, H₂O+0.1% HCOOH/MeCN+0.1% HCOOH 80:20 to 0:100) and freeze dried with water to obtain Example 1 (137 mg, 54% over 2 steps) as a white solid.

¹H-NMR (DMSO-d₆, 300 MHz) δ: 1.69 (s, 6H, 2CH₃); 1.83-2.00 (m, 4H, 2CH₂, CH₂CH₂O); 3.52-3.58 (m, 2H, CH₂); 3.93-4.09 (m, 4H, 2CH₂, CH₂CH₂O); 7.38 (d, J 8.4 Hz, 1H, Ar); 7.43 (ddd, J 8.4, 2.1, 0.9 Hz, 1H, Ar); 7.57 (d, J 8.4 Hz, 1H, Ar); 7.69 (dd, J 8.4, 2.1 Hz, 1H, Ar); 7.83 (t, J 8.4 Hz, 1H, Ar); 8.13-8.16 (m, 1H, NH). One CH₂ signal not observed.

M/Z (M[³⁵Cl]+H)⁺: 487.0,

Mp: 162-164° C.

### Compound 22: 1-(3-bromo-6-chloropyridin-2-yl)cyclopentane-1-carbonitrile

Compound 22 was prepared according to general procedure (I) starting from 3-bromo-6-chloro-2-fluoropyridine (5.00 g) and cyclopentanecarbonitrile. The crude was purified by flash chromatography (Merck 60®, CyHex 100% to CyHex/Et₂O 80:20) to obtain Compound 22 (5.57 g, 82%) as a white solid.

M/Z (M[³⁵Cl][⁸¹Br]+H)⁺: 287.0

### Compound 23: 1-(3-bromo-6-chloropyridin-2-yl)cyclopentane-1-carboxamide

Compound 23 was prepared according to general procedure (IIa) starting from Compound 22 (5.57 g). The crude was purified by flash chromatography (Merck 60®, CyHex 100% to CyHex/EtOAc 0:100) to obtain Compound 23 (5.96 g) as a white solid.

M/Z (M[³⁵Cl⁸¹Br]+H)⁺:305.1

### Compound 24: 5′-chlorospiro[cyclopentane-1,3′-pyrrolo[3,2-b]pyridin]-2′(1′H)-one

Compound 24 was prepared according to general procedure (III) starting from Compound 23 (5.96 g). The crude was purified by flash chromatography (Merck 60®, CyHex 100% to CyHex/EtOAc 50:50) to obtain Compound 24 (3.37 g, 77% over 2 steps) as a beige solid.

M/Z (M[³⁵Cl]+H)⁺:223.0

### Compound 25: 2′-oxo-1′,2′-dihydrospiro[cyclopentane-1,3′-pyrrolo[3,2-b]pyridine]-5′-carbonitrile

Compound 25 was prepared according to general procedure (IV) starting from Compound 24 (200 mg). The crude was purified by flash chromatography (Merck 60®, CyHex 100% to CyHex/EtOAc 0:100) to obtain Compound 25 (184 mg, 96%) as a white solid.

M/Z (M+H)⁺: 214.1.

### Compound 26: 1′-(4-chloro-3-fluorophenyl)-2′-oxo-1′,2′-dihydrospiro[cyclopentane-1,3′-pyrrolo[3,2-b]pyridine]-5′-carbonitrile

Compound 26 was prepared according to general procedure (V) starting from Compound 25 (184 mg) and 4-bromo-1-chloro-2-fluorobenzene (199 mg, 1.1 eq.). The crude was purified by flash chromatography (Merck 60®, CyHex 100% to CyHex/EtOAc 50:50) to obtain Compound 26 (250 mg, 89%) as a beige solid.

M/Z (M[³⁵Cl]+H)⁺:342.1.

### Compound 27: 1′-(4-chloro-3-fluorophenyl)-2′-oxo-1′,2′-dihydrospiro[cyclopentane-1,3′-pyrrolo[3,2-b]pyridine]-5′-carboxamide

Compound 27 was prepared according to general procedure (IIa) starting from Compound 26 (220 mg). The crude was purified by flash chromatography (Merck 60®, CyHex 100% to CyHex/EtOAc 0:100) to obtain Compound 27 (240 mg) as a white solid.

M/Z (M[³⁵Cl]+H)⁺: 360.2

### Compound 28: methyl 1′-(4-chloro-3-fluorophenyl)-2′-oxospiro[cyclopentane-1,3′-indoline]-5′-carboxylate

Compound 28 was prepared according to general procedure (VI) starting from Compound 27 (240 mg). The crude was purified by flash chromatography (Merck 60®, CyHex 100% to CyHex/EtOAc 50:50) to obtain Compound 28 (212 mg, 88% over 2 steps) as a white solid.

M/Z (M [³⁵Cl]+H)⁺: 375.2.

### Compound 29: 1′-(4-chloro-3-fluorophenyl)-2′-oxo-1′,2′-dihydrospiro[cyclopentane-1,3′-pyrrolo[3,2-b]pyridine]-5′-carboxylic acid

Compound 29 was prepared according to general procedure (VII) starting from Compound 28 (212 mg). The crude was purified by flash chromatography (Merck 60®, DCM 100% to DCM/MeOH 80:20), then further purified by preparative HPLC (Column B, H₂O+0.1% HCOOH/MeCN+0.1% HCOOH 80:20 to 0:100) and freeze-dried to obtain Compound 29 (55 mg, 27%) as a white solid.

M/Z (M ³⁵Cl+H)⁺361.2.

### Example 2: 1′-(4-chloro-3-fluorophenyl)-5′-(2,2-dimethyl-3-oxopiperazine-1-carbonyl)spiro[cyclopentane-1,3′-pyrrolo[3,2-b]pyridin]-2′(1′H)-one

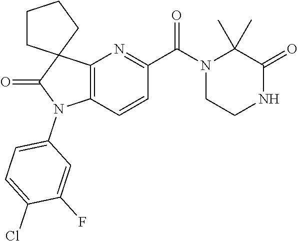 [SMILES: CC1(C)C(=O)NCCN1C(=O)c1ccc2c(n1)C1(CCCC1)C(=O)N2c1ccc(Cl)c(F)c1]

Example 2 was prepared according to general procedure (VIIIa) starting from Compound 29 (55 mg), 3,3-dimethylpiperazin-2-one (1.3 eq.) and N,N-diisopropylethylamine (3.0 eq.) in a mixture DCM/THF. The crude was purified by flash chromatography (Merck 60®, CyHex 100% to EtOAc 100%) then freeze dried with MeOH/water to obtain Example 2 (30 mg, 42%) as a white solid.

¹H-NMR (DMSO-d₆, 300 MHz) δ: 1.67 (s, 6H, 2CH₃); 1.64-2.15 (m, 8H, 4CH₂); 3.47-3.53 (m, 2H, CH₂); 7.33 (d, J 8.1 Hz, 1H, Ar); 7.39-7.73 (m, 1H, Ar); 7.50 (d, J 8.1 Hz, 1H, Ar); 7.69 (dd, J 10.2, J 2.4 Hz, 1H, Ar); 7.80 (t, J 8.4 Hz, 1H, Ar); 8.06-8.09 (m, 1H, NHCO), one CH₂ signal not observed.

M/Z (M[³⁵Cl]+H)⁺: 471.3.

Mp: 143 145° C.

### Compound 30: 5′-chloro-1′,2′-dihydrospiro[cyclopentane-1,3′-pyrrolo[3,2-b]pyridine]

Compound 30 was prepared according to general procedure (IX) starting from Compound 24 (1.60 g). The crude was purified by flash chromatography (Merck 60®, CyHex 100% to CyHex/EtOAc 60:40) to obtain Compound 30 (850 mg, 56%) as a beige solid.

M/Z (M[³⁵Cl]+H)⁺: 209.2

### Compound 31: 1′,2′-dihydrospiro[cyclopentane-1,3′-pyrrolo[3,2-b]pyridine]-5′-carbonitrile

Compound 31 was prepared according to general procedure (IV) starting from Compound 30 (850 mg). The crude was purified by flash chromatography (Merck 60®, CyHex 100% to CyHex/EtOAc 50:50) to obtain Compound 31 (602 mg, 74%) as a brown solid

M/Z (M+H)⁺: 200.2

### Compound 32: 1′-(4-chloro-3-fluorophenyl)-1′,2′-dihydrospiro[cyclopentane-1,3′-pyrrolo[3,2-b]pyridine]-5′-carbonitrile

Compound 32 was prepared according to general procedure (X) starting from Compound 31 (195 mg) and 4-bromo-1-chloro-2-fluorobenzene (1.1 eq.). The crude was purified by flash chromatography (Merck 60®, CyHex 100% to CyHex/EtOAc 80:20) to obtain Compound 32 (220 mg) as a yellow solid.

M/Z (M[³⁵Cl]+H)⁺: 328.2

### Compound 33: 1′-(4-chloro-3-fluorophenyl)-1′,2′-dihydrospiro[cyclopentane-1,3′-pyrrolo[3,2-b]pyridine]-5′-carboxamide

Compound 33 was prepared according to general procedure (IIa) starting from Compound 32 (220 mg). The crude was purified by flash chromatography (Merck 60®, CyHex 100% to CyHex/EtOAc 0:100) to obtain Compound 33 (200 mg, 60% over 2 steps) as a beige solid.

M/Z (M[³⁵Cl]+H)⁺: 346.2

### Compound 34: methyl 1′-(4-chloro-3-fluorophenyl)-1′,2′-dihydrospiro[cyclopentane-1,3′-pyrrolo[3,2-b]pyridine]-5′-carboxylate

Compound 34 was prepared according to general procedure (VI) starting from Compound 33 (200 mg). The crude was purified by flash chromatography (Merck 60®, CyHex 100% to CyHex/EtOAc 0:100) to obtain Compound 34 (197 mg, 94%) as a beige solid.

M/Z (M[³⁵Cl]+H)⁺: 361.3

### Compound 35: 1′-(4-chloro-3-fluorophenyl)-1′,2′-dihydrospiro[cyclopentane-1,3′-pyrrolo[3,2-b]pyridine]-5′-carboxylic acid

Compound 35 was obtained (255 mg) as a yellow solid according to general procedure (VII) starting from Compound 34 (251 mg).

M/Z (M[³⁵Cl]+H)⁺: 347.3

### Example 3: 4-(1′-(4-chloro-3-fluorophenyl)-1′,2′-dihydrospiro[cyclopentane-1,3′-pyrrolo[3,2-b]pyridine]-5′-carbonyl)-3,3-dimethylpiperazin-2-one

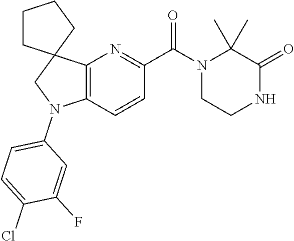 [SMILES: CC1(C)C(=O)NCCN1C(=O)c1ccc2c(n1)C1(CCCC1)CN2c1ccc(Cl)c(F)c1]

Example 3 was prepared according to general procedure (VIIIa) starting from Compound 35 (99 mg), 3,3-dimethylpiperazin-2-one (2.0 eq.) and N,N-diisopropylethylamine (3.0 eq.) in a THF/DCM mixture (5:2). The crude was purified by flash chromatography (Merck 60®, CyHex 100% to EtOAc 100%) then triturated in 12 mL of a DCM/Pentane mixture (1:5) to obtain Example 3 (48 mg, 32% over 2 steps) as a white solid.

¹H-NMR (DMSO-d₆, 300 MHz) δ: 1.66 (s, 6H, 2CH₃); 1.81-1.93 (m, 8H, 4CH₂); 3.56-3.51 (m, 2H, CH₂); 3.93 (s, 2H, CH₂); 7.19 (ddd, J 8.7, 2.7, 1.0 Hz, 1H, Ar), 7.33 (dd, J 11.8, 2.7 Hz, 1H, Ar); 7.40 (d, J 8.4 Hz, 1H, Ar); 7.51-7.57 (m, 2H, 2Ar), 8.02-8.06 (m, 1H, NH).

M/Z (M[³⁵Cl]+H)⁺: 457.3.

Mp: 198:200° C.

### Example 4: 8-(1′-(4-chloro-3-fluorophenyl)-1′,2′-dihydrospiro[cyclopentane-1,3′-pyrrolo[3,2-b]pyridine]-5′-carbonyl)-1,3,8-triazaspiro[4.5]decane-2,4-dione

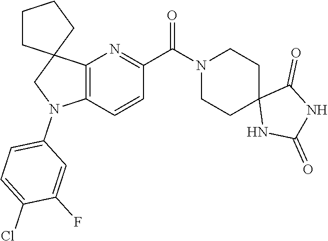 [SMILES: O=C1NC(=O)C2(CCN(C(=O)c3ccc4c(n3)C3(CCCC3)CN4c3ccc(Cl)c(F)c3)CC2)N1]

Example 4 was prepared according to general procedure (VIIIa) starting from Compound 35 (50 mg), 1,3,8-triazaspiro[4.5]decane-2,4-dione (2.0 eq.) and N,N-diisopropylethylamine (3.0 eq.) in DCM. The crude was purified by preparative HPLC (Column B, H₂O+0.1% HCOOH/MeCN+0.1% HCOOH 95:05 to 0:100) then freeze dried to obtain Example 4 (16 mg, 22% over 2 steps) as a white solid.

¹H-NMR (DMSO-d₆, 300 MHz) δ: 1.54-2.01 (m, 12H, 4CH₂ cyclopentyl+2 (N—CH₂—CH₂); 3.17-3.27 (m, 1H, N—CH₂—CH₂); 3.39-3.51 (m, 1H, N—CH₂—CH₂); 3.93 (s, 2H, C—CH₂—N); 3.97-4.09 (m, 1H, N—CH₂—CH₂); 4.18-4.32 (m, 1H, N—CH₂—CH₂); 7.17-7.21 (dd, J 8.9, 2.0 Hz, 1H, Ar); 7.30-7.35 (dd, J 11.9, 2.5 Hz, 1H, Ar); 7.40 (d, J 8.4 Hz, 1H, Ar); 7.49-7.59 (m, 2H, 2Ar); 8.58 (s, 1H, NH); 10.74 (bs, 1H, NH).

M/Z (M[³⁵Cl]+H)⁺: 498.2.

Mp: 280-285° C.

### Example 5: methyl 1-(1′-(4-chloro-3-fluorophenyl)-1′,2′-dihydrospiro[cyclopentane-1,3′-pyrrolo[3,2-b]pyridine]-5′-carbonyl)-3,3-dimethylpiperidine-4-carboxylate

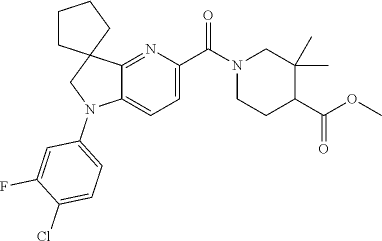 [SMILES: COC(=O)C1CCN(C(=O)c2ccc3c(n2)C2(CCCC2)CN3c2ccc(Cl)c(F)c2)CC1(C)C]

Example 5 was prepared according to general procedure (VIIIa) starting from Compound 35 (80 mg), methyl 3,3-dimethylpiperidine-4-carboxylate (2.0 eq.) and N,N-diisopropylethylamine (3.0 eq.) in DCM. The crude was purified by flash chromatography (Merck 60®, CyHex 100% to CyHex/EtOAc 50:50) to obtain Example 5 (100 mg) as a yellow oil.

¹H-NMR (CDCl₃, 300 MHz) δ: 0.81-1.12 (m, 6H, 2CH₃); 1.40-1.45 (m, 4H, 2CH₂ cyclopentyl); 1.69-2.22 (m, 6H, 2CH₂ cyclopentyl+N—CH₂—CH₂), 2.36-2.48 (1H, m, CH), 2.81-3.23 (m, 2H, NCH₂), 3.70 (s, 3H, OCH₃), 3.83 (s, 2H, N—CH₂), 4.26-4.61 (m, 2H, N—CH₂), 6.92-7.02 (m, 2H, 2Ar), 7.28-7.37 (m, 2H, 2Ar), 7.51 (d, J 8.4 Hz, 1H, Ar).

M/Z (M[³⁵Cl]+H)⁺: 500.3.

### Example 6: 1-(1′-(4-chloro-3-fluorophenyl)-1′,2′-dihydrospiro[cyclopentane-1,3′-pyrrolo[3,2-b]pyridine]-5′-carbonyl)-3,3-dimethylpiperidine-4-carboxylic acid

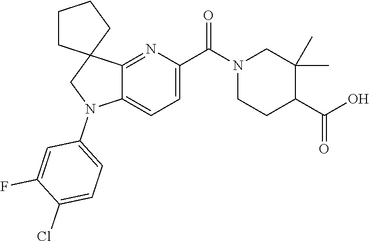 [SMILES: CC1(C)CN(C(=O)c2ccc3c(n2)C2(CCCC2)CN3c2ccc(Cl)c(F)c2)CCC1C(=O)O]

Example 6 was prepared according to general procedure (VII) starting from Example 5 (100 mg). The crude was purified by flash chromatography (Merck 60®, DCM 100% to DCM/MeOH 80:20) then further purified by preparative HPLC (Column B, (H₂O+0.1% HCOOH/MeCN+0.1% HCOOH 95:05 to 0:100) and freeze dried to obtain Example 6 (20 mg, 18% over 3 steps) as a white solid.

¹H-NMR (DMSO-d₆, 300 MHz) δ: 0.77-1.10 (m, 6H, 2CH3); 1.53-2.03 (m, 10H, 4CH2 cyclopentyl+N—CH2-CH2); 2.28-2.41 (m, 1H, CO—CH—C(CH3)2); 2.77-3.14 (m, 2H, N—CH2), 3.86-4.12 (m, 3.5H, N—CH2+N—CH2-cyclopentyl); 4.33-4.45 (m, 0.5H, N—CH2); 7.19 (dd, J 8.4, 2.0 Hz, 1H, Ar); 7.28-7.41 (m, 2H, 2Ar); 7.49-7.58 (m, 2H, 2Ar); One signal not observed (COOH).

M/Z (M[³⁵Cl]+H)⁺: 486.3.

Mp: 280-285° C.

### Example 7: methyl 6-(4-(1′-(4-chloro-3-fluorophenyl)-1′,2′-dihydrospiro[cyclopentane-1,3′-pyrrolo[3,2-b]pyridine]-5′-carbonyl)-3,3-dimethylpiperazin-1-yl)-2,4-dimethylnicotinate

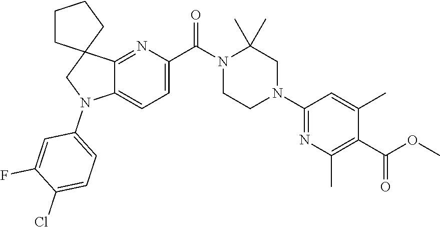 [SMILES: COC(=O)c1c(C)cc(N2CCN(C(=O)c3ccc4c(n3)C3(CCCC3)CN4c3ccc(Cl)c(F)c3)C(C)(C)C2)nc1C]

Example 7 was prepared according to general procedure (VIIIa) starting from Compound 35 (105 mg), Compound 10 (1.2 eq.) and N,N-diisopropylethylamine (4.0 eq.) in THF/DCM mixture (1:1). The crude was purified by flash chromatography (Merck 60®, CyHex 100% to CyHex/EtOAc 50:50) to obtain Example 7 (155 mg, 84%) as a white solid.

M/Z (M[³⁵Cl]+H)⁺: 606.5

### Example 8: 6-(4-(1′-(4-chloro-3-fluorophenyl)-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid

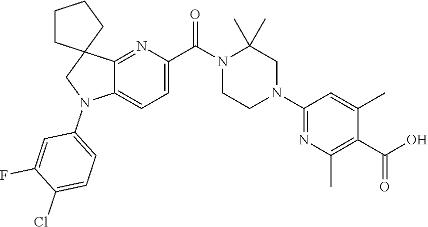 [SMILES: Cc1cc(N2CCN(C(=O)c3ccc4c(n3)C3(CCCC3)CN4c3ccc(Cl)c(F)c3)C(C)(C)C2)nc(C)c1C(=O)O]

Example 8 was prepared according to general procedure (XI) starting from Example 7 (155 mg). The crude was purified by flash chromatography (Merck 60®, DCM 100% to DCM/MeOH 80:20) and freeze dried to obtain Example 8 (10 mg, 6%) as a white solid.

¹H-NMR (DMSO-d₆ 300 MHz) δ: 1.47 (s, 6H, 2CH₃); 1.74-2.01 (m, 8H, 4CH₂); 2.25 (s, 3H, CH₃); 2.36 (s, 3H, CH₃); 3.52-3.59 (m, 2H, CH₂); 3.77-3.95 (m, 6H, 3CH₂); 6.30 (s, 1H, Ar), 7.18 (dd, J 2.1, 8.7 Hz, 1H, Ar); 7.29-7.35 (m, 2H, 2Ar); 7.50-7.57 (m, 2H, 2Ar). COOH was not observed.

M/Z (M[³⁵Cl]+H)⁺: 592.4.

Mp: 265-267° C.

### Compound 36: 1′-(3-chlorophenyl)-1′,2′-dihydrospiro[cyclopentane-1,3′-pyrrolo[3,2-b]pyridine]-5′-carbonitrile

Compound 36 was prepared according to general procedure (X) starting from Compound 31 (100 mg) and 1-bromo-3-chlorobenzene (1.1 eq.). The crude was purified by flash chromatography (Merck 60®, CyHex 100% to CyHex/EtOAc 80:20) to obtain Compound 36 (110 mg) as a yellow oil.

M/Z (M[³⁵Cl]+H)⁺: 310.7

### Compound 37: 1′-(3-chlorophenyl)-1′,2′-dihydrospiro[cyclopentane-1,3′-pyrrolo[3,2-b]pyridine]-5′-carboxamide

Compound 37 was prepared according to general procedure (IIa) starting from Compound 36 (110 mg). The crude was purified by flash chromatography (Merck 60®, CyHex 100% to CyHex/EtOAc 20:80) to obtain Compound 37 (70 mg) as a white solid.

M/Z (M[³⁵Cl]+H)⁺: 328.3

### Compound 38: methyl 1′-(3-chlorophenyl)-1′,2′-dihydrospiro[cyclopentane-1,3′-pyrrolo[3,2-b]pyridine]-5′-carboxylate

Compound 38 was prepared according to general procedure (VI) starting from Compound 36 (70 mg). The crude was purified by flash chromatography (Merck 60®, CyHex 100% to CyHex/EtOAc 40:60) to obtain Compound 38 (40 mg) as a white solid.

M/Z (M[³⁵Cl]+H)⁺: 343.1

### Compound 39: 1′-(3-chlorophenyl)-1′,2′-dihydrospiro[cyclopentane-1,3′-pyrrolo[3,2-b]pyridine]-5′-carboxylic acid

Compound 39 was obtained (40 mg) as a yellow solid according to general procedure (VII) starting from Compound 38 (40 mg).

M/Z (M[³⁵Cl]+H)⁺: 329.0

### Example 9: 4-(1′-(3-chlorophenyl)-1′,2′-dihydrospiro[cyclopentane-1,3′-pyrrolo[3,2-b]pyridine]-5′-carbonyl)-3,3-dimethylpiperazin-2-one

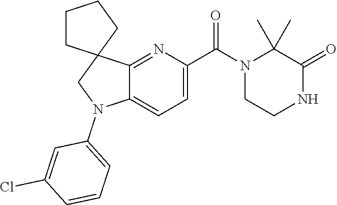 [SMILES: CC1(C)C(=O)NCCN1C(=O)c1ccc2c(n1)C1(CCCC1)CN2c1cccc(Cl)c1]

Example 9 was prepared according to general procedure (VIIIa) starting from Compound 39 (40 mg), 3,3-dimethylpiperazin-2-one (2.0 eq.) and N,N-diisopropylethylamine (3.0 eq.) in DCM. The crude was purified by flash chromatography (Merck 60®, CyHex 100% to Cyhex/EtOAc 70:30) to obtain Example 9 (50 mg, 22% over 5 steps) as a white solid.

¹H-NMR (DMSO-d₆, 300 MHz) δ: 1.66 (s, 6H, 2CH₃); 1.73-2.00 (m, 8H, 4CH₂); 3.51-3.59 (m, 2H, CH₂); 3.94 (s, 2H, CH₂); 7.07 (d, J 7.5 Hz, 1H, Ar); 7.28-7.33 (m, 2H, 2Ar); 7.38-7.42 (m, 2H, 2Ar); 7.48 (d, J 7.5 Hz, 1H, Ar); 8.04 (bs, 1H, CONHR), one CH₂ signal not observed.

M/Z (M[³⁵Cl]+H)⁺: 439.4.

Mp: 188-192° C.

### Compound 40: 1′-(2-chlorophenyl)-1′,2′-dihydrospiro[cyclopentane-1,3′-pyrrolo[3,2-b]pyridine]-5′-carbonitrile

Compound 40 was prepared according to general procedure (X) starting from Compound 31 (100 mg) and 1-bromo-2-chlorobenzene (1.1 eq.). The crude was purified by flash chromatography (Merck 60®, CyHex 100% to CyHex/EtOAc 80:20) to obtain Compound 40 (90 mg) as a yellow oil.

M/Z (M[³⁵Cl]+H)⁺: 310.2

### Compound 41: 1′-(2-chlorophenyl)-1′,2′-dihydrospiro[cyclopentane-1,3′-pyrrolo[3,2-b]pyridine]-5′-carboxamide

Compound 41 was prepared according to general procedure (IIa) starting from Compound 40 (90 mg). The crude was purified by flash chromatography (Merck 60®, CyHex 100% to CyHex/EtOAc 20:80) to obtain Compound 41 (70 mg) as a white solid.

M/Z (M[³⁵Cl]+H)⁺: 328.3

### Compound 42: methyl 1′-(2-chlorophenyl)-1′,2′-dihydrospiro[cyclopentane-1,3′-pyrrolo[3,2-b]pyridine]-5′-carboxylate

Compound 42 was prepared according to general procedure (VI) starting from Compound 41 (90 mg). The crude was purified by flash chromatography (Merck 60®, CyHex 100% to CyHex/EtOAc 0:100) to obtain Compound 42 (50 mg) as a white solid.

M/Z (M[³⁵Cl]+H)⁺: 343.1

### Compound 43: 1′-(2-chlorophenyl)-1′,2′-dihydrospiro[cyclopentane-1,3′-pyrrolo[3,2-b]pyridine]-5′-carboxylic acid

Compound 43 was prepared according to general procedure (VII) starting from Compound 42 (50 mg). The crude was purified by flash chromatography (Merck 60®, CyHex 100% to CyHex/EtOAc 30:70) to obtain Compound 43 (44 mg) as a white solid.

M/Z (M[³⁵Cl]+H)⁺: 329.1

### Example 10: 4-(1′-(2-chlorophenyl)-1′,2′-dihydrospiro[cyclopentane-1,3′-pyrrolo[3,2-b]pyridine]-5′-carbonyl)-3,3-dimethylpiperazin-2-one

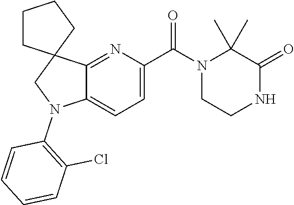 [SMILES: CC1(C)C(=O)NCCN1C(=O)c1ccc2c(n1)C1(CCCC1)CN2c1ccccc1Cl]

Example 10 was prepared according to general procedure (VIIIa) starting from Compound 43 (44 mg), 3,3-dimethylpiperazin-2-one (2.0 eq.) and N,N-diisopropylethylamine (5.0 eq.) in DCM. The crude was purified by flash chromatography (Merck 60®, CyHex 100% to Cyhex/EtOAc 70:30) to obtain Example 10 (14 mg, 6% over 5 steps) as a white solid.

¹H-NMR (DMSO-d₆, 300 MHz) δ: 1.64 (s, 6H, 2CH₃); 1.63-2.03 (m, 8H, 4CH₂); 3.53-3.62 (m, 2H, CH₂); 3.88 (s, 2H, CH₂); 6.55 (d, J 8.5 Hz, 1H, Ar); 7.30-7.36 (m, 2H, 2Ar); 7.44 (td, J 8.0, 1.2 Hz, 1H, Ar); 7.55 (dd, J 8.0, 1.2 Hz, 1H, Ar); 7.61 (dd, J 8.0, 1.2 Hz, 1H, Ar); 8.04 (bs, 1H, CONHR); one CH₂ signal not observed.

M/Z (M[³⁵Cl]+H)⁺: 439.3.

Mp: 206-210° C.

### Compound 44: 2-(3-bromo-6-chloropyridin-2-yl)-2-methylpropanenitrile

Compound 44 was prepared according to general procedure (I) starting from 3-bromo-6-chloro-2-fluoropyridine (5.00 g) and isobutyronitrile (2.13 mL). The crude was purified by flash chromatography (Merck 60®, CyHex 100% to CyHex/EtOAc 80:20) to obtain Compound 44 (4.14 g) as a clear oil.

M/Z (M[³⁵Cl][⁸¹Br]+H)⁺: 261.0

### Compound 45: 2-(3-bromo-6-chloropyridin-2-yl)-2-methylpropanamide

Compound 45 was prepared according to general procedure (IIa) starting from Compound 44 (4.14 g). The crude was triturated in a mixture DCM/n-pentane (20/80). The precipitate was recovered by filtration to obtain Compound 45 (2.77 g, 42% over 2 steps) as a white solid.

M/Z (M[³⁵Cl⁸¹Br]+H)⁺: 279.0

### Compound 46: 5-chloro-3,3-dimethyl-1,3-dihydro-2H-pyrrolo[3,2-b]pyridin-2-one

Compound 46 was prepared according to general procedure (III) starting from Compound 45 (2.77 g). The crude was purified by flash chromatography (Merck 60®, CyHex 100% to CyHex/EtOAc 0:100) to obtain Compound 46 (1.57 g, 80%) as a beige solid.

M/Z (M[³⁵Cl]+H)⁺: 197.0

### Compound 47: 5-chloro-3,3-dimethyl-2,3-dihydro-1H-pyrrolo[3,2-b]pyridine

Compound 47 was prepared according to general procedure (IX) starting from Compound 46 (1.57 g). The crude was purified by flash chromatography (Merck 60®, CyHex 100% to CyHex/EtOAc 0:100) to obtain Compound 47 (1.09 g, 75%) as a beige solid.

M/Z (M[³⁵Cl]+H)⁺: 182.9

### Compound 48: 3,3-dimethyl-2,3-dihydro-1H-pyrrolo[3,2-b]pyridine-5-carbonitrile

Compound 48 was prepared according to general procedure (IV) starting from Compound 47 (1.09 g). The crude was purified by flash chromatography (Merck 60®, CyHex 100% to CyHex/EtOAc 20:80) to obtain Compound 48 (460 mg, 45%) as a beige solid

M/Z (M+H)⁺: 174.2

### Compound 49: 3,3-dimethyl-1-(thiophen-3-yl)-2,3-dihydro-1H-pyrrolo[3,2-b]pyridine-5-carbonitrile

Compound 49 was prepared according to general procedure (X) starting from Compound 48 (100 mg) and 3-bromothiophene (1.6 eq.). The crude was purified by flash chromatography (Merck 60®, CyHex 100% to CyHex/EtOAc 80:20) to obtain Compound 49 (100 mg, 68%) as a yellow solid.

M/Z (M+H)⁺: 256.2

### Compound 50: 3,3-dimethyl-1-(thiophen-3-yl)-2,3-dihydro-1H-pyrrolo[3,2-b]pyridine-5-carboxamide

Compound 50 was prepared according to general procedure (IIa) starting from Compound 49 (100 mg). The crude was purified by flash chromatography (Merck 60®, CyHex 100% to CyHex/EtOAc 0:100) to obtain Compound 50 (120 mg) as a beige solid.

M/Z (M+H)⁺: 274.2

### Compound 51: methyl 3,3-dimethyl-1-(thiophen-3-yl)-2,3-dihydro-1H-pyrrolo[3,2-b]pyridine-5-carboxylate

Compound 51 was prepared according to general procedure (VI) starting from Compound 50 (120 mg). The crude was purified by flash chromatography (Merck 60®, CyHex 100% to CyHex/EtOAc 0:100) to obtain Compound 51 (60 mg) as a yellow solid.

M/Z (M+H)⁺: 289.2

### Compound 52: 3,3-dimethyl-1-(thiophen-3-yl)-2,3-dihydro-1H-pyrrolo[3,2-b]pyridine-5-carboxylic acid

Compound 52 was obtained (60 mg) as a yellow solid according to general procedure (VII) starting from Compound 51 (60 mg).

M/Z (M+H)⁺: 275.1

### Example 11: 4-(3,3-dimethyl-1-(thiophen-3-yl)-2,3-dihydro-1H-pyrrolo[3,2-b]pyridine-5-carbonyl)-3,3-dimethylpiperazin-2-one

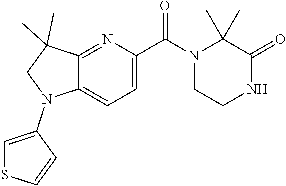 [SMILES: CC1(C)CN(c2ccsc2)c2ccc(C(=O)N3CCNC(=O)C3(C)C)nc21]

Example 11 was prepared according to general procedure (VIIIa) starting from Compound 52 (60 mg), 3,3-dimethylpiperazin-2-one (2.0 eq.) and N,N-diisopropylethylamine (3.0 eq.) in DCM. The crude was purified by flash chromatography (Merck 60®, CyHex 100% to CyHex/EtOAc 0:100) then further purified by preparative HPLC (Column B, H₂O+0.1% HCOOH/MeCN+0.1% HCOOH 80:20 to 0:100) and freeze dried to obtain Example 11 (11 mg, 8% over 4 steps) as a beige solid.

¹H-NMR (DMSO-d₆, 300 MHz) δ: 1.33 (s, 6H, 2CH₃); 1.66 (s, 6H, 2CH₃); 3.34-3.38 (m, 2H, CH₂); 3.50-3.57 (m, 2H, CH₂); 3.84 (s, 2H, CH₂); 7.07 (dd, J 3.1, 1.6 Hz, 1H, Ar); 7.30 (dd, J 5.1, 1.6 Hz, 1H, Ar); 7.39 (s, 2H, 2Ar); 7.64 (dd, J 5.1, 3.1 Hz, 1H, Ar); 8.04 (bs, 1H, CONHR).

M/Z (M+H)⁺: 385.2.

Mp: 208-212° C.

### Compound 53: 1-(3,4-difluorophenyl)-3,3-dimethyl-2,3-dihydro-1H-pyrrolo[3,2-b]pyridine-5-carbonitrile

Compound 53 was prepared according to general procedure (X) starting from Compound 48 (85 mg) and 4-bromo-1,2-difluorobenzene (1.0 eq). The crude was purified by flash chromatography (Merck 60®, CyHex 100% to CyHex/EtOAc 0:100) to obtain Compound 53 (110 mg, 79%) as a yellow oil.

M/Z (M+H)⁺: 286.2

### Compound 54: 1-(3,4-difluorophenyl)-3,3-dimethyl-2,3-dihydro-1H-pyrrolo[3,2-b]pyridine-5-carboxamide

Compound 54 was prepared according to general procedure (IIa) starting from Compound 53 (110 mg). The crude was purified by flash chromatography (Merck 60®, CyHex 100% to CyHex/EtOAc 0:100) to obtain Compound 54 (100 mg) as a white solid.

M/Z (M+H)⁺: 304.2

### Compound 55: methyl 1-(3,4-difluorophenyl)-3,3-dimethyl-2,3-dihydro-1H-pyrrolo[3,2-b]pyridine-5-carboxylate

Compound 55 was prepared according to general procedure (VI) starting from Compound 54 (120 mg). The crude was purified by flash chromatography (Merck 60®, CyHex 100% to CyHex/EtOAc 20:80) to obtain Compound 55 (85 mg, 81%) as a yellow solid.

M/Z (M+H)⁺: 319.1

### Compound 56: 1-(3,4-difluorophenyl)-3,3-dimethyl-2,3-dihydro-1H-pyrrolo[3,2-b]pyridine-5-carboxylic acid

Compound 56 was obtained (80 mg, 98%) as a yellow solid according to general procedure (VII) starting from Compound 55 (85 mg).

M/Z (M+H)⁺: 305.1

### Example 12: 4-(1-(3,4-difluorophenyl)-3,3-dimethyl-2,3-dihydro-1H-pyrrolo[3,2-b]pyridine-5-carbonyl)-3,3-dimethylpiperazin-2-one

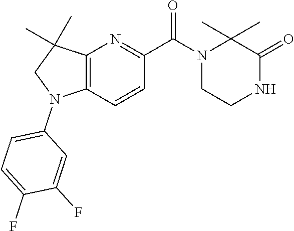 [SMILES: CC1(C)CN(c2ccc(F)c(F)c2)c2ccc(C(=O)N3CCNC(=O)C3(C)C)nc21]

Example 12 was prepared according to general procedure (VIIIa) starting from Compound 56 (40 mg), 3,3-dimethylpiperazin-2-one (1.2 eq.) and N,N-diisopropylethylamine (3.0 eq.) in DCM. The crude was purified by flash chromatography (Merck 60®, CyHex 100% to CyHex/EtOAc 0:100) then triturated in Et₂O to obtain Example 12 (30 mg, 55%) as a white solid.

¹H-NMR (DMSO-d₆, 300 MHz) δ: 1.33 (s, 6H, 2CH₃); 1.66 (s, 6H, 2CH₃); 3.31-3.35 (m, 2H, CH₂); 3.51-3.54 (m, 2H, CH₂); 3.84 (s, 2H, CH₂); 7.12-7.17 (m, 1H, Ar); 7.33-7.50 (m, 4H, 4Ar); 8.05 (bs, 1H, NH).

M/Z (M+H)⁺: 415.2.

Mp: 217-220° C.

### Example 13: methyl 6-(4-(1-(3,4-difluorophenyl)-3,3-dimethyl-2,3-dihydro-1H-pyrrolo[3,2-b]pyridine-5-carbonyl)-3,3-dimethylpiperazin-1-yl)-2,4-dimethylnicotinate

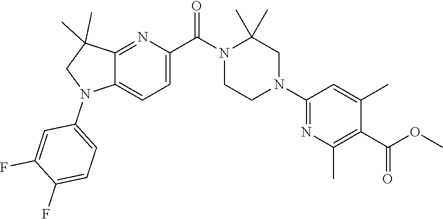 [SMILES: COC(=O)c1c(C)cc(N2CCN(C(=O)c3ccc4c(n3)C(C)(C)CN4c3ccc(F)c(F)c3)C(C)(C)C2)nc1C]

Example 13 was prepared according to general procedure (VIIIa) starting from Compound 56 (47 mg), Compound 10 (1.2 eq.) and N,N-diisopropylethylamine (3.0 eq.) in DCM. The crude was purified by flash chromatography (Merck 60®, CyHex 100% to CyHex/EtOAc 0:100) to obtain Example 13 (77 mg, 88%) as a white solid.

¹H-NMR (CDCl₃, 300 MHz) δ: 1.43 (s, 6H, 2CH₃); 1.63 (s, 6H, 2CH₃); 2.34 (s, 3H, CH₃); 2.49 (s, 3H, CH₃); 3.61-3.67 (m, 2H, CH₂); 3.77 (s, 2H, N—CH₂); 3.89 (s, 3H, O—CH₃); 3.96-4.02 (m, 4H, 2N—CH₂); 6.11 (s, 1H, Ar); 6.90-6.96 (m, 1H, Ar); 7.00-7.07 (m, 1H, Ar); 7.14-7.24 (m, 1H, Ar); 7.28 (d, J 8.4 Hz, 1H, Ar); 7.50 (d, J 8.4 Hz, 1H, Ar).

M/Z (M+H)⁺: 564.4

### Example 14: 6-(4-(1-(3,4-difluorophenyl)-3,3-dimethyl-2,3-dihydro-1H-pyrrolo[3,2-b]pyridine-5-carbonyl)-3,3-dimethylpiperazin-1-yl)-2,4-dimethylnicotinic acid

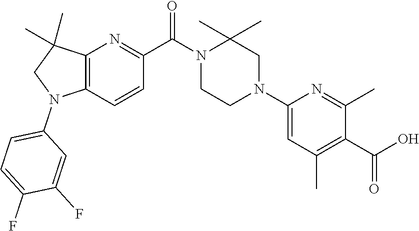 [SMILES: Cc1cc(N2CCN(C(=O)c3ccc4c(n3)C(C)(C)CN4c3ccc(F)c(F)c3)C(C)(C)C2)nc(C)c1C(=O)O]

Example 14 was prepared according to general procedure (XI) starting from Example 13 (77 mg). The crude was purified by flash chromatography (Merck 60®, DCM 100% to DCM/MeOH 80:20) and freeze dried to obtain Example 14 (40 mg, 53%) as a white solid.

¹H-NMR (DMSO-d₆ 300 MHz) δ: 1.34 (s, 6H, 2CH₃); 1.48 (s, 6H, 2CH₃); 2.25 (s, 3H, CH₃); 2.36 (s, 3H, CH₃); 3.52- 3.55 (m, 2H, CH₂); 3.80-3.86 (m, 6H, 3CH₂); 6.32 (s, 1H, Ar); 7.11-7.16 (m, 1H, Ar); 7.31-7.40 (m, 2H, 2Ar); 7.43-7.48 (m, 2H, 2Ar), 12.68 (bs, 1H, CO₂H).

M/Z (M+H)⁺: 550.3

### Compound 57: 1-(4-fluorophenyl)-3,3-dimethyl-2,3-dihydro-1H-pyrrolo[3,2-b]pyridine-5-carbonitrile

Compound 57 was prepared according to general procedure (X) starting from Compound 48 (85 mg) and 1-bromo-4-fluorobenzene (1.0 eq). The crude was purified by flash chromatography (Merck 60®, CyHex 100% to CyHex/EtOAc 0:100) to obtain Compound 57 (86 mg, 66%) as a yellow solid.

M/Z (M+H)⁺: 268.2

### Compound 58: 1-(4-fluorophenyl)-3,3-dimethyl-2,3-dihydro-1H-pyrrolo[3,2-b]pyridine-5-carboxamide

Compound 58 (90 mg, 99%) was obtained as a white solid from Compound 57 (85 mg) according to general procedure (IIb).

M/Z (M+H)⁺: 286.2

### Compound 59: methyl 1-(4-fluorophenyl)-3,3-dimethyl-2,3-dihydro-1H-pyrrolo[3,2-b]pyridine-5-carboxylate

Compound 59 was prepared according to general procedure (VI) starting from Compound 58 (120 mg). The crude was purified by flash chromatography (Merck 60®, CyHex 100% to CyHex/EtOAc 60:40) to obtain Compound 59 (85 mg, 81%) as a white solid.

M/Z (M+H)⁺: 301.2

### Compound 60: 1-(4-fluorophenyl)-3,3-dimethyl-2,3-dihydro-1H-pyrrolo[3,2-b]pyridine-5-carboxylic acid

Compound 60 was obtained (80 mg) as a yellow solid according to general procedure (VII) starting from Compound 59 (85 mg).

M/Z (M+H)⁺: 287.2

### Example 15: 4-(1-(4-fluorophenyl)-3,3-dimethyl-2,3-dihydro-1H-pyrrolo[3,2-b]pyridine-5-carbonyl)-3,3-dimethylpiperazin-2-one

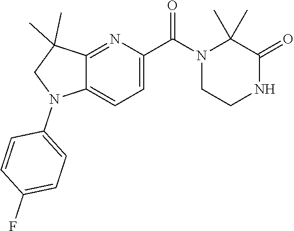 [SMILES: CC1(C)CN(c2ccc(F)cc2)c2ccc(C(=O)N3CCNC(=O)C3(C)C)nc21]

Example 15 was prepared according to general procedure (VIIIa) starting from Compound 60 (33 mg), 3,3-dimethylpiperazin-2-one (1.2 eq.) and N,N-diisopropylethylamine (3.0 eq.) in DCM. The crude was purified by flash chromatography (Merck 60®, CyHex 100% to CyHex/EtOAc 0:100) then triturated in Et₂O to obtain Example 15 (20 mg, 44%) as a white solid.

¹H-NMR (DMSO-d₆, 300 MHz) δ: 1.32 (s, 6H, 2CH₃); 1.65 (s, 6H, 2CH₃); 3.32-3.35 (m, 2H, CH₂); 3.51-3.54 (m, 2H, CH₂); 3.82 (s, 2H, CH₂); 7.20-7.26 (m, 2H, 2Ar); 7.30-7.38 (m, 4H, 4Ar); 8.04 (bs, 1H, NH).

M/Z (M+H)⁺: 397.2.

Mp: 234-236° C.

### Compound 61: 1-(3-fluorophenyl)-3,3-dimethyl-2,3-dihydro-1H-pyrrolo[3,2-b]pyridine-5-carbonitrile

Compound 61 was prepared according to general procedure (X) starting from Compound 48 (150 mg) and 1-bromo-3-fluorobenzene (1.0 eq). The crude was purified by flash chromatography (Merck 60®, CyHex 100% to CyHex/EtOAc 50:50) to obtain Compound 61 (205 mg, 89%) as a white solid.

M/Z (M+H)⁺: 268.2

### Compound 62: 1-(3-fluorophenyl)-3,3-dimethyl-2,3-dihydro-1H-pyrrolo[3,2-b]pyridine-5-carboxamide

Compound 62 (210 mg, 98%) was obtained as a white solid from Compound 61 (200 mg) according to general procedure (IIb).

M/Z (M+H)⁺: 286.2

### Compound 63: methyl 1-(3-fluorophenyl)-3,3-dimethyl-2,3-dihydro-1H-pyrrolo[3,2-b]pyridine-5-carboxylate

Compound 63 was prepared according to general procedure (VI) starting from Compound 62 (210 mg). The crude was purified by flash chromatography (Merck 60®, CyHex 100% to CyHex/EtOAc 60:40) to obtain Compound 63 (160 mg, 72%) as a white solid.

M/Z (M+H)⁺: 301.2

### Compound 64: 1-(3-fluorophenyl)-3,3-dimethyl-2,3-dihydro-1H-pyrrolo[3,2-b]pyridine-5-carboxylic acid

Compound 64 was obtained (140 mg, 92%) as a yellow solid according to general procedure (VII) starting from Compound 63 (160 mg).

M/Z (M+H)⁺: 287.2

### Example 16: methyl 6-(4-(1-(3-fluorophenyl)-3,3-dimethyl-2,3-dihydro-1H-pyrrolo[3,2-b]pyridine-5-carbonyl)-3,3-dimethylpiperazin-1-yl)-2,4-dimethylnicotinate

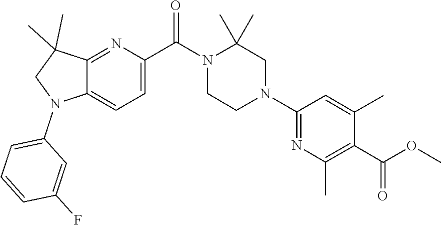 [SMILES: COC(=O)c1c(C)cc(N2CCN(C(=O)c3ccc4c(n3)C(C)(C)CN4c3cccc(F)c3)C(C)(C)C2)nc1C]

Example 16 was prepared according to general procedure (VIIIb) starting from Compound 64 (50 mg), Compound 10 (1.3 eq.) and Triethylamine (4.7 eq.) in THF. The crude was purified by flash chromatography (Merck 60®, CyHex 100% to Et₂O 0:100). The product was further purified by dissolution in Et₂O (10 mL), extraction with HCl (aq. 1N, 3*10 mL), aqueous layers were basified with Na₂CO₃ (sat. aq.) and extracted with Et₂O (3*10 mL) to obtain Example 16 (60 mg, 63%) as a white solid.

¹H-NMR (CDCl₃, 300 MHz) δ: 1.43 (s, 6H, 2CH₃); 1.63 (s, 6H, 2CH₃); 2.34 (s, 3H, CH₃); 2.49 (s, 3H, CH₃); 3.61-3.67 (m, 2H, CH₂); 3.81 (s, 2H, N—CH₂); 3.89 (s, 3H, O—CH₃); 3.97 (s, 2H, N—CH₂); 3.99-4.03 (m, 2H, N—CH₂); 6.11 (s, 1H, Ar); 6.71-6.78 (m, 1H, Ar); 6.89-6.95 (m, 1H, Ar); 6.98-7.01 (m, 1H, Ar); 7.27-7.38 (m, 1H, Ar); 7.40 (d, J 8.4 Hz, 1H, Ar); 7.51 (d, J 8.4 Hz, 1H, Ar).

M/Z (M+H)⁺: 546.4

### Example 17: 6-(4-(1-(3-fluorophenyl)-3,3-dimethyl-2,3-dihydro-1H-pyrrolo[3,2-b]pyridine-5-carbonyl)-3,3-dimethylpiperazin-1-yl)-2,4-dimethylnicotinic acid

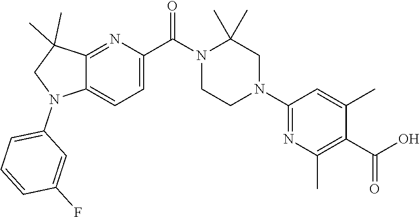 [SMILES: Cc1cc(N2CCN(C(=O)c3ccc4c(n3)C(C)(C)CN4c3cccc(F)c3)C(C)(C)C2)nc(C)c1C(=O)O]

Example 17 was prepared according to general procedure (XI) starting from Example 16 (60 mg). The crude was purified by flash chromatography (Merck 60®, DCM 100% to DCM/MeOH 80:20) and freeze dried to obtain Example 17 (41 mg, 70%) as a white solid.

¹H-NMR (DMSO-d₆ 300 MHz) δ: 1.35 (s, 6H, 2CH₃); 1.48 (s, 6H, 2CH₃); 2.24 (s, 3H, CH₃); 2.35 (s, 3H, CH₃); 3.51- 3.54 (m, 2H, CH₂); 3.79-3.85 (m, 6H, 3CH₂); 6.28 (bs, 1H, Ar); 6.84 (dt, J 6.3, 2.1 Hz, 1H, Ar); 7.06-7.17 (m, 2H, 2 Ar); 7.33 (d, J 8.2 Hz, 1H, Ar); 7.37-7.45 (m, 1H, Ar); 7.53 (d, J 8.2 Hz, 1H, Ar). COOH was not observed.

M/Z (M+H)⁺: 532.4

### Compound 65: 1-(4-chloro-3-fluorophenyl)-3,3-dimethyl-2,3-dihydro-1H-pyrrolo[3,2-b]pyridine-5-carbonitrile

Compound 65 was prepared according to general procedure (X) starting from Compound 48 (100 mg) and 4-bromo-1-chloro-2-fluorobenzene (1.1 eq.). The crude was purified by flash chromatography (Merck 60®, CyHex 100% to CyHex/EtOAc 80:20) to obtain Compound 65 (230 mg, 88%) as a yellow solid.

M/Z (M[³⁵Cl]+H)⁺: 302.2.

### Compound 66: 1-(4-chloro-3-fluorophenyl)-3,3-dimethyl-2,3-dihydro-1H-pyrrolo[3,2-b]pyridine-5-carboxamide

Compound 66 was prepared according to general procedure (IIa) starting from Compound 65 (230 mg). The crude was purified by flash chromatography (Merck 60®, CyHex 100% to CyHex/EtOAc 0:100) to obtain Compound 66 (300 mg) as a yellow solid.

M/Z (M[³⁵Cl]+H)⁺: 320.2.

### Compound 67: methyl 1-(4-chloro-3-fluorophenyl)-3,3-dimethyl-2,3-dihydro-1H-pyrrolo[3,2-b]pyridine-5-carboxylate

Compound 67 was prepared according to general procedure (VI) starting from Compound 66 (300 mg). The crude was purified by flash chromatography (Merck 60®, CyHex 100% to CyHex/EtOAc 40:60) to obtain Compound 67 (140 mg, 55% over 2 steps) as a white solid.

M/Z (M[³⁵Cl]+H)⁺: 335.2.

### Compound 68: 1-(4-chloro-3-fluorophenyl)-3,3-dimethyl-2,3-dihydro-1H-pyrrolo[3,2-b]pyridine-5-carboxylic acid

Compound 68 was obtained (140 mg) as a yellow solid according to general procedure (VII) starting from Compound 67 (140 mg).

M/Z (M[³⁵Cl]+H)⁺: 321.2.

### Compound 69: 6-chloro-2-iodo-N-(2-methylallyl)pyridin-3-amine

To a solution of 6-chloro-2-iodopyridin-3-amine (5.00 g) in THF, ^{t}BuOK (1.2 eq) was added at 0° C. The reaction mixture was stirred at 0° C. for 10 minutes then 3-Bromo-2-methylpropene (1.1 eq) was added and the reaction was stirred at 25° C. for 16 hours. The reaction mixture was quenched with NH4Cl (150 mL, sat. aq.), extracted with EtOAc (2*100 mL). Combined organic layers were washed with brine (150 mL), dried over MgSO₄ and concentrated under reduced pressure. The crude was purified by flash chromatography (Interchim®50 μm, CyHex 100% to CyHex/EtOAc 80:20) to obtain Compound 69 (4.54 g, 75%) as an orange oil.

M/Z (M[³⁵Cl]+H)⁺: 309.1.

### Compound 70: 5-chloro-3,3-dimethyl-2,3-dihydro-1H-pyrrolo[3,2-b]pyridine

To a solution of Compound 69 (1.51 g) in a DMSO/water mixture (16.5 mL, 9:1) was added tetrabutylammonium chloride (1.2 eq), sodium formate (1.2 eq) and triethylamine (3.0 eq). The reaction mixture was sparged with argon for 10 minutes then Palladium acetate (0.15 eq) was added and the reaction vial was crimped and heated at 120° C. for 1 hour. The reaction mixture was filtered over a Celite pad that was further rinsed with EtOAc (50 mL). The filtrate was washed with NH4Cl (50 mL, sat.aq.) then with brine (50 mL), dried over MgSO₄, and concentrated under reduced pressure. The crude was purified by flash chromatography (Interchim®50 μm, CyHex 100% to CyHex/EtOAc 70/30) to obtain Compound 70 (0.70 g, 78%) as a yellow solid.

M/Z (M[³⁵Cl]+H)⁺: 183.1.

### Compound 71: 5-chloro-1-(4-chloro-3-fluorophenyl)-3,3-dimethyl-2,3-dihydro-1H-pyrrolo[3,2-b]pyridine

Compound 71 was prepared according to general procedure (X) starting from Compound 70 (600 mg) and 4-bromo-1-chloro-2-fluorobenzene (2.0 eq) with ^{t}BuONa (3.0 eq) at 100° C. for 16 hours. The crude was purified by flash chromatography (Interchim®50 μm, CyHex 100% to CyHex/DCM 70:30) to obtain Compound 71 (925 mg, 91%) as a beige solid.

M/Z (M[³⁵Cl]₂+H)⁺: 311.17.

### Example 18: 4-(1-(4-chloro-3-fluorophenyl)-3,3-dimethyl-2,3-dihydro-1H-pyrrolo[3,2-b]pyridine-5-carbonyl)-3,3-dimethylpiperazin-2-one

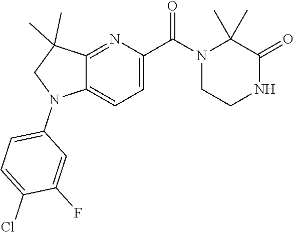 [SMILES: CC1(C)CN(c2ccc(Cl)c(F)c2)c2ccc(C(=O)N3CCNC(=O)C3(C)C)nc21]

Example 18 was prepared according to general procedure (VIIIa) starting from Compound 68 (70 mg), 3,3-dimethylpiperazin-2-one (2.0 eq.) and N,N-diisopropylethylamine (3.0 eq.) in DCM. The crude was purified by flash chromatography (Merck 60®, CyHex 100% to CyHex/EtOAc 0:100) then further purified by preparative HPLC (Column B, H₂O+0.1% HCOOH/MeCN+0.1% HCOOH 80:20 to 0:100) and freeze dried to obtain Example 18 (39 mg, 43% over 2 steps) as a white solid.

¹H-NMR (DMSO-d₆, 300 MHz) δ: 1.32 (s, 6H, 2CH₃); 1.66 (s, 6H, 2CH₃); 3.48-3.55 (m, 2H, CH₂); 3.86 (s, 2H, CH₂); 7.18 (dd, J 8.5, 2.5 Hz, 1H, Ar); 7.37 (dd, J 1.7, 2.5 Hz, 1H, Ar); 7.40 (d, J 8.2 Hz, 1H, Ar); 7.52-7.58 (m, 2H, 2Ar); 8.06 (bs, 1H, CONHR); one CH₂ signal not observed.

M/Z (M[³⁵Cl]+H)⁺: 431.2.

Mp: 249-253° C.

### Example 19: 4-(1-(4-chloro-3-fluorophenyl)-3,3-dimethyl-2,3-dihydro-1H-pyrrolo[3,2-b]pyridine-5-carbonyl)-1-ethyl-3,3-dimethylpiperazin-2-one

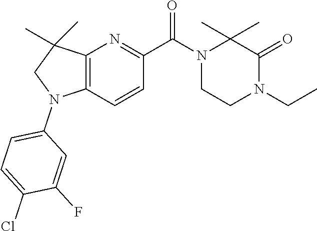 [SMILES: CCN1CCN(C(=O)c2ccc3c(n2)C(C)(C)CN3c2ccc(Cl)c(F)c2)C(C)(C)C1=O]

Example 19 was prepared according to general procedure (XIII) starting from Example 18 (70 mg), and bromoethane (1.8 eq.) in THF. The crude was purified by flash chromatography (Merck 60®, CyHex 100% to CyHex/EtOAc 0:100) to obtain Example 19 (45 mg, 60%) as a white solid.

¹H-NMR (DMSO-d₆, 300 MHz) δ: 1.05 (t, J 6.9 Hz, 3H, CH₃); 1.33 (s, 6H, (CH₃)₂); 1.66 (s, 6H, (CH₃)₂); 3.35 (d, J 6.9 Hz, 2H, CH₂); 3.47-3.51 (m, 2H, CH₂); 3.56-3.59 (m, 2H, CH₂); 3.86 (s, 2H, CH₂); 7.18 (m, 1H, Ar); 7.33 (dd, J 11.7, 2.4 Hz, 1H, Ar); 7.41 (d, J 8.4 Hz, 1H); 7.52-7.58 (m, 2H, 2Ar).

M/Z (M[³⁵Cl]+H)⁺: 459.5.

Mp: 224-226° C.

### Example 20: (1-(4-chloro-3-fluorophenyl)-3,3-dimethyl-2,3-dihydro-1H-pyrrolo[3,2-b]pyridin-5-yl)(3,3-dimethylmorpholino)methanone

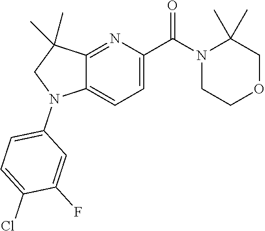 [SMILES: CC1(C)CN(c2ccc(Cl)c(F)c2)c2ccc(C(=O)N3CCOCC3(C)C)nc21]

Example 20 was prepared according to general procedure (VIIIa) starting from Compound 68 (70 mg), 3,3-dimethylmorpholine (2.0 eq.) and N,N-diisopropylethylamine (3.0 eq.) in DCM. The crude was purified by flash chromatography (Merck 60®, CyHex 100% to CyHex/EtOAc 0:100) then freeze dried to obtain Example 20 (39 mg, 45% over 2 steps) as a white solid.

¹H-NMR (DMSO-d₆, 300 MHz) δ: 1.32 (s, 6H, 2CH₃); 1.39 (s, 6H, 2CH₃); 3.41 (s, 2H, CH₂); 3.46-3.50 (m, 2H, CH₂); 3.69-3.73 (m, 2H, CH₂); 3.85 (s, 2H, CH₂); 7.18 (dd, J 8.7, 2.4 Hz, 1H, Ar); 7.29-7.36 (m, 2H, 2Ar); 7.51-7.58 (m, 2H, 2Ar).

M/Z (M[³⁵Cl]+H)⁺: 418.3.

Mp: 173-177° C.

### Example 21: tert-butyl 4-(1-(4-chloro-3-fluorophenyl)-3,3-dimethyl-2,3-dihydro-1H-pyrrolo[3,2-b]pyridine-5-carbonyl)-3,3-dimethylpiperazine-1-carboxylate

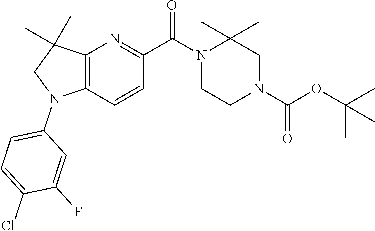 [SMILES: CC(C)(C)OC(=O)N1CCN(C(=O)c2ccc3c(n2)C(C)(C)CN3c2ccc(Cl)c(F)c2)C(C)(C)C1]

Example 21 was prepared according to general procedure (VIIIa) starting from Compound 68 (500 mg), tert-butyl 3,3-dimethylpiperazine-1-carboxylate (1.0 eq.) and N,N-diisopropylethylamine (4.0 eq.) in DCM. The crude was purified by flash chromatography (Merck 60®, CyHex 100% to CyHex/EtOAc 50:50) to obtain Example 21 (740 mg, 92%) as a clear oil.

¹H-NMR (DMSO-d₆, 300 MHz) δ: 1.32 (s, 6H, (CH₃)₂); 1.41 (s, 9H, (CH₃)₃); 1.45 (s, 6H, (CH₃)₂); 3.35-3.45 (m, 4H, (CH₂)₂); 3.46-3.55 (m, 2H, CH₂); 3.84 (s, 2H, CH₂); 7.16 (dd, J 9.0, 2.1 Hz, 1H, Ar); 7.27-7.35 (m, 2H, 2Ar); 7.51-7.57 (m, 2H, 2Ar).

M/Z (M[³⁵Cl]+H)⁺: 517.3.

### Example 22: (1-(4-chloro-3-fluorophenyl)-3,3-dimethyl-2,3-dihydro-1H-pyrrolo[3,2-b]pyridin-5-yl)(2,2-dimethylpiperazin-1-yl)methanone hydrochloride

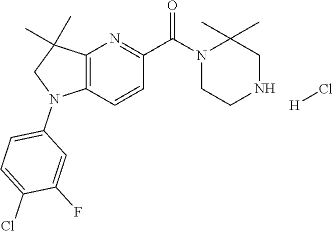

Example 22 was prepared according to general procedure (XII) starting from Example 21 (690 mg) in dioxane with HCl in dioxane. The crude was purified by trituration in Et₂O (30 mL) to obtain Example 22 (600 mg, 99%) as a yellow solid.

¹H-NMR (DMSO-d₆, 300 MHz) δ: 1.32 (s, 6H, (CH₃)₂); 1.54 (s, 6H, (CH₃)₂); 3.13-3.17 (m, 2H, N—CH₂); 3.25-3.30 (m, 2H, N—CH₂); 3.64-3.68 (m, 2H, N—CH₂); 7.16-7.20 (m, 1H, Ar); 7.33 (dd, J 12.0, 2.7 Hz, 1H, Ar); 7.43 (d, J 8.4 Hz, 1H, Ar); 7.52-7.58 (m, 2H, 2Ar); 9.24 (bs, 2H, NH₂+). One N—CH₂ was not observed.

M/Z (M[³⁵Cl]+H)⁺: 417.2.

### Example 23: (1-(4-chloro-3-fluorophenyl)-3,3-dimethyl-2,3-dihydro-1H-pyrrolo[3,2-b]pyridin-5-yl)(4-ethyl-2,2-dimethylpiperazin-1-yl)methanone hydrochloride

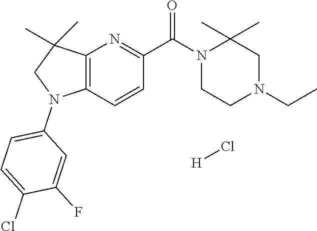

To a suspension of Example 22 (70 mg) in MeTHF (2.0 mL) was added triethylamine (2.0 eq), acetaldehyde (1.0 eq), sodium triacetoxyborohydride (2.0 eq). The reaction mixture was stirred at 25° C. for 18 hours, then quenched with NaHCO₃ (sat. aq. 10 mL) and extracted with EtOAc (10 mL). The crude was purified by preparative HPLC (H₂O+0.1% HCOOH/MeCN+0.1% HCOOH 80:20 to 40:60) and freeze dried with aqueous HCl to obtain Example 23 (22 mg, 30%) as a yellow solid.

¹H-NMR (DMSO-d₆, 300 MHz) δ: 1.29 (t, J 7.2 Hz, 3H, CH₃); 1.32 (s, 3H, CH₃); 1.36 (s, 3H, CH₃); 1.55 (s, 3H, CH₃); 1.60 (s, 3H, CH3); 3.05-3.22 (m, 4H, (CH₂)₂); 3.36-3.46 (m, 2H, CH₂); 3.52-3.56 (m, 1H, CH); 3.83-3.90 (m, 2H, CH₂); 4.06-4.11 (m, 1H, CH); 7.17-7.21 (m, 1H, Ar); 7.33 (dd, J 11.7; 2.7 Hz, 1H, Ar); 7.46 (d, J 8.4 Hz, 1H, Ar); 7.53-7.58 (m, 2H, 2Ar); 10.21 (bs, 1H, NH⁺).

M/Z (M[³⁵Cl]+H)⁺: 445.4

### Example 24: 1-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)ethan-1-one

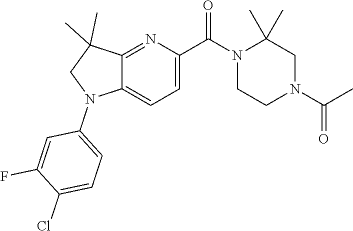 [SMILES: CC(=O)N1CCN(C(=O)c2ccc3c(n2)C(C)(C)CN3c2ccc(Cl)c(F)c2)C(C)(C)C1]

Example 24 was prepared according to general procedure (VIIIa) starting from Example 22 (75 mg), acetic acid (1.0 eq.) and N,N-diisopropylethylamine (4.0 eq.) in DCM. The crude was purified by flash chromatography (Interchim® 50 μm, CyHex 100% to CyHex/EtOAc 0:100) then further purified by preparative HPLC (H₂O+0.1% HCOOH/MeCN+0.1% HCOOH 80:20 to 40:60) and freeze dried with aqueous HCl to obtain Example 24 (30 mg, 42%) as a yellow solid.

¹H-NMR (DMSO-d₆, 300 MHz) δ: 1.32-1.34 (m, 6H, (CH₃)₂); 1.45 (s, 3H, CH₃); 1.49 (s, 3H, CH₃); 1.97-2.03 (m, 3H, CH₃); 3.43-3.47 (m, 1H, CH); 3.56 (d, J 6.3 Hz, 2H, CH₂); 3.60-3.67 (m, 2H, CH₂); 3.69-3.72 (m, 1H, CH); 3.84 (s, 2H, CH₂); 7.17 (dd, J 8.7, 2.4 Hz, 1H, Ar); 7.28-7.36 (m, 2H, 2Ar); 7.51-7.58 (m, 2H, 2Ar).

M/Z (M[³⁵Cl]+H)⁺: 459.4

### Example 25: ethyl 4-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-oxobutanoate

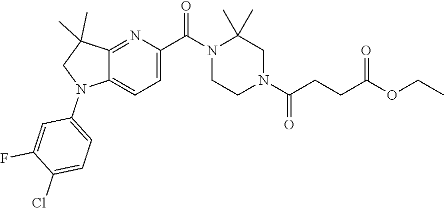 [SMILES: CCOC(=O)CCC(=O)N1CCN(C(=O)c2ccc3c(n2)C(C)(C)CN3c2ccc(Cl)c(F)c2)C(C)(C)C1]

Example 25 was prepared according to general procedure (VIIIa) starting from Example 22 (75 mg), 4-ethoxy-4-oxobutanoic acid (1.0 eq.) and N,N-diisopropylethylamine (4.0 eq.) in DCM. The crude was purified by flash chromatography (Interchim®50 μm, CyHex 100% to CyHex/EtOAc 0:100) and freeze dried to obtain Example 25 (65 mg, 77%) as a white solid.

¹H-NMR (DMSO-d₆, 300 MHz) δ: 1-13-1.18 (m, 3H, OCH₂—CH₃); 1.32-1.33 (m, 6H, (CH₃)₂); 1.44 (s, 3H, CH₃); 1.50 (s, 3H, CH₃); 2.53-2.61 (m, 4H, 2CH₂); 3.45-3.99 (m, 6H, 3N—CH₂); 3.84 (s, 2H, N—CH₂); 3.98-4.06 (m, 2H, O—CH₂); 7.15-7.19 (m, 1H, Ar). 7.29-7.36 (m, 2H, 2Ar) 7.51-7.57 (m, 2H, 2Ar).

M/Z (M[³⁵Cl]+H)⁺: 545.5.

### Example 26: 4-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-oxobutanoic acid

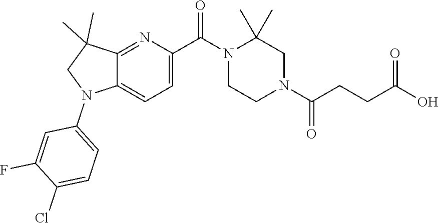 [SMILES: CC1(C)CN(c2ccc(Cl)c(F)c2)c2ccc(C(=O)N3CCN(C(=O)CCC(=O)O)CC3(C)C)nc21]

Example 26 was prepared according to general procedure (VII) starting from Example 25 (65 mg). The crude was purified by preparative HPLC (Column B, H₂O+0.1% HCOOH/MeCN+0.1% HCOOH 80:20 to 0:100) and freeze dried with aqueous HCl to obtain Example 26 (35 mg, 57%) as a yellow solid.

¹H-NMR (DMSO-d₆, 300 MHz) δ: 1.32-1.33 (m, 6H, (CH₃)₂); 1.45 (s, 3H, CH₃); 1.50 (s, 3H, CH₃); 2.42-2.48 (m, 2H, CH₂); 2.54-2.63 (m, 2H, CH₂); 3.56-3.59 (m, 2H, CH₂); 3.64-3.72 (m, 4H, (CH₂)₂); 3.64 (s, 2H, CH₂); 7.15-7.19 (m, 1H, Ar). 7.29-7.36 (m, 2H, 2Ar) 7.51-7.57 (m, 2H, 2Ar). COOH was not observed.

M/Z (M[³⁵Cl]+H)⁺: 545.5

### Example 27: ethyl 5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-5-oxopentanoate

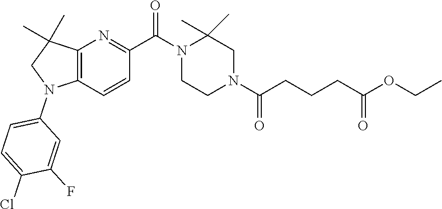 [SMILES: CCOC(=O)CCCC(=O)N1CCN(C(=O)c2ccc3c(n2)C(C)(C)CN3c2ccc(Cl)c(F)c2)C(C)(C)C1]

Example 27 was prepared according to general procedure (VIIIa) starting from Example 22 (75 mg), 5-ethoxy-5-oxopentanoic acid (1.0 eq.) and N,N-diisopropylethylamine (4.0 eq.) in DCM. The crude was purified by flash chromatography (Interchim®50 μm, CyHex 100% to CyHex/EtOAc 0:100) and freeze dried to obtain Example 27 (66 mg, 77%) as a white solid.

¹H-NMR (DMSO-d₆, 300 MHz) δ: 1.13-1.19 (m, 3H, CH₃); 1.32 (s, 3H, CH₃); 1.32 (s, 3H, CH₃); 1.44 (s, 3H, CH₃); 1.48 (s, 3H, CH₃); 1.73-1.79 (m, 2H, CH₂); 2.31-2.34 (m, 4H, (CH₂)₂; 3.43-3.47 (m, 1H, CH); 3.55-3.70 (m, 5H, (CH₂)₂, CH); 3.84 (s, 2H, CH₂); 3.99-4.08 (m, 2H, CH₂); 7.17 (dd, J 9.0, 2.1 Hz, 1H, Ar); 7.29-7.36 (m, 2H, 2Ar); 7.51-7.59 (m, 2H, 2Ar).

M/Z (M[³⁵Cl]+H)⁺: 559.5

### Example 28: 5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-5-oxopentanoic acid

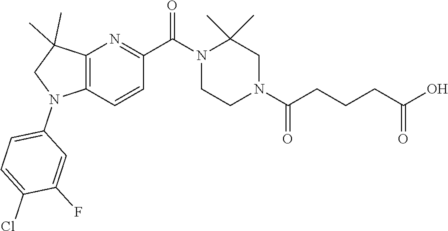 [SMILES: CC1(C)CN(c2ccc(Cl)c(F)c2)c2ccc(C(=O)N3CCN(C(=O)CCCC(=O)O)CC3(C)C)nc21]

Example 28 was prepared according to general procedure (VII) starting from Example 27 (59 mg). The crude was purified by preparative HPLC (Column B, H₂O+0.1% HCOOH/MeCN+0.1% HCOOH 80:20 to 0:100) and freeze dried with aqueous HCl to obtain Example 28 (30 mg, 54%) as a yellow solid.

¹H-NMR (DMSO-d₆, 300 MHz) δ: 1.32 (s, 3H, CH₃); 1.33 (s, 3H, CH₃); 1.45 (s, 3H, CH₃); 1.46 (s, 3H, CH₃); 1.70-1.76 (m, 2H, CH₂); 2.23-2.37 (m, 4H, (CH₂)₂); 3.44-3.47 (m, 1H, CH); 3.56-3.70 (m, 5H, (CH₂)₂+CH); 3.04 (s, 2H, CH₂); 7.17 (dd, J 9.0, 2.4 Hz, 1H, Ar); 7.29-7.37 (m, 2H, 2Ar); 7.51-7.57 (m, 2H, 2Ar). COOH was not observed.

M/Z (M[³⁵Cl]+H)⁺: 531.5.

### Example 29: 8-(1-(4-chloro-3-fluorophenyl)-3,3-dimethyl-2,3-dihydro-1H-pyrrolo[3,2-b]pyridine-5-carbonyl)-1-methyl-1,3,8-triazaspiro[4.5]decan-4-one

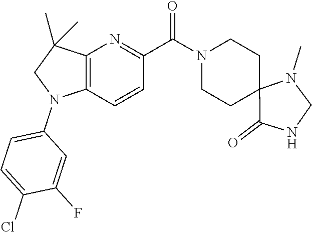 [SMILES: CN1CNC(=O)C12CCN(C(=O)c1ccc3c(n1)C(C)(C)CN3c1ccc(Cl)c(F)c1)CC2]

Example 29 was prepared according to general procedure (VIIIa) starting from Compound 68 (75 mg), 1-methyl-1,3,8-triazaspiro[4.5]decan-4-one hydrochloride (1.5 eq.) and N,N-diisopropylethylamine (4.0 eq.) in DCM. The crude was purified by preparative HPLC (H₂O+0.1% HCOOH/MeCN+0.1% HCOOH 80:20 to 40:60) and freeze dried with water to obtain Example 29 (40 mg, 36%) as a white solid.

¹H-NMR (DMSO-d₆, 300 MHz) δ: 1.33 (s, 6H, 2*CH₃); 1.51-1.88 (m, 4H, 2*CH₂); 2.20-2.38 (m, 3H, N—CH₃); 3.44-3.58 (m, 1H, N—CHₐH_{b}); 3.64-3.77 (m, 1H, N—CHₐH_{b}); 3.80-3.92 (m, 3H, 3*N—CHₐH_{b}); 3.94-4.08 (m, 2H, 2*N—CHₐH_{b}); 4.16-4.29 (m, 1H, N—CHₐH_{b}); 7.15-7.22 (m, 1H, Ar); 7.33 (dd, J 11.9 2.6 Hz, 1H, Ar); 7.40 (d, J 8.4 Hz, 1H, Ar); 7.51-7.59 (m, 2H, 2Ar); 8.28 (bs, 1H, NH).

M/Z (M[³⁵Cl]+H)⁺: 472.3.

Example 30: methyl 2-((1R,5S,6S)-3-(1-(4-chloro-3-fluorophenyl)-3,3-dimethyl-2,3-dihydro-1H-pyrrolo[3,2-b]pyridine-5-carbonyl)-3-azabicyclo[3.1.0]hexan-6-yl)acetate

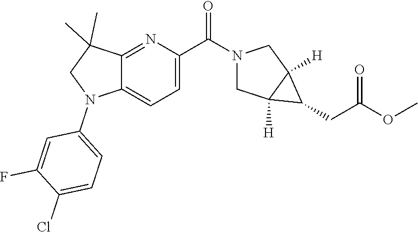 [SMILES: COC(=O)C[C@@H]1[C@H]2CN(C(=O)c3ccc4c(n3)C(C)(C)CN4c3ccc(Cl)c(F)c3)C[C@@H]12]

Example 30 was prepared according to general procedure (VIIIa) starting from Compound 68 (62 mg), methyl 2-((1R,5S,6S)-3-azabicyclo[3.1.0]hexan-6-yl)acetate hydrochloride (44 mg, 1.2 eq) and N,N-diisopropylethylamine (3.0 eq.) in DCM. The crude was purified by flash chromatography (Interchim® 50 μm, CyHex 100% to CyHex/EtOAc 20:80) to obtain Example 30 (75 mg, 85%) as a white solid.

¹H-NMR (DMSO-d₆, 300 MHz) δ: 0.63-0.70 (m, 1H, CH); 1.33 (s, 3H, CH₃); 1.36 (s, 3H, CH₃); 1.43-1.53 (m, 2H, 2*CH); 2.28-2.33 (m, 2H, CH₂); 3.4-3.48 (m, 1H, CHₐH_{b}); 3.59 (s, 3H, O—CH₃); 3.87 (s, 2H, N—CH₂); 3.88-3.98 (m, 3H, N—CH₂+N—CHₐH_{b}); 7.16-7.21 (m, 1H, Ar); 7.31-7.35 (m, 1H, Ar); 7.50-7.59 (m, 3H, 3Ar).

M/Z (M[³⁵Cl]+H)⁺: 458.3

### Example 31: 2-((1R,5S,6S)-3-(1-(4-chloro-3-fluorophenyl)-3,3-dimethyl-2,3-dihydro-1H-pyrrolo[3,2-b]pyridine-5-carbonyl)-3-azabicyclo[3.1.0]hexan-6-yl)acetic acid

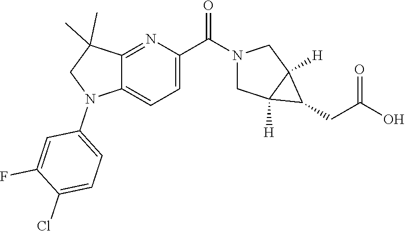 [SMILES: CC1(C)CN(c2ccc(Cl)c(F)c2)c2ccc(C(=O)N3C[C@@H]4[C@@H](CC(=O)O)[C@@H]4C3)nc21]

Example 31 was prepared according to general procedure (VII) starting from Example 30 (72 mg). The crude was purified by preparative HPLC (Column B, H₂O+0.1% HCOOH/MeCN+0.1% HCOOH 20:80 to 0:100) and freeze dried to obtain Example 31 (48 mg, 69%) as a white solid.

¹H-NMR (DMSO-d₆, 300 MHz) δ: 0.60-0.70 (m, 1H, CH); 1.33 (s, 3H, CH₃); 1.36 (s, 3H, CH₃); 1.42-1.54 (m, 2H, 2*CH); 2.18-2.24 (m, 2H, CH₂); 3.40-3.48 (m, 1H, CHaHb); 3.87 (s, 2H, N—CH₂); 3.88-3.98 (m, 3H, N—CH₂+N—CHaHb); 7.16-7.21 (m, 1H, Ar); 7.33 (dd, J 11.9, 2.6 Hz, 1H, Ar); 7.50-7.59 (m, 3H, 3Ar); 12.09 (bs, 1H, COOH).

M/Z (M[³⁵Cl]+H)⁺: 444.3

### Example 32: ethyl 1-(1-(4-chloro-3-fluorophenyl)-3,3-dimethyl-2,3-dihydro-1H-pyrrolo[3,2-b]pyridine-5-carbonyl)piperidine-4-carboxylate

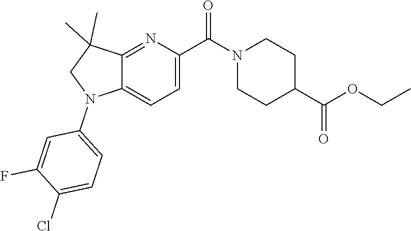 [SMILES: CCOC(=O)C1CCN(C(=O)c2ccc3c(n2)C(C)(C)CN3c2ccc(Cl)c(F)c2)CC1]

Example 32 was prepared according to general procedure (VIIIa) starting from Compound 68 (62 mg), ethyl piperidine-4-carboxylate (37 mg, 1.2 eq) and N,N-diisopropylethylamine (3.0 eq.) in DCM. The crude was purified by flash chromatography (Interchim®50 μm, CyHex 100% to CyHex/EtOAc 20:80) to obtain Example 32 (38 mg, 43%) as a white solid.

¹H-NMR (DMSO-d₆, 300 MHz) δ: 1.19 (t, J 6.9 Hz, 3H, O—CH₂—CH₃); 1.33 (s, 6H, 2*CH₃); 1.42-1.66 (m, 2H, CH₂); 1.78-2.00 (m, 2H, CH₂); 2.58-2.66 (m, 1H, CH); 2.88-3.04 (m, 1H, N—CHₐH_{b}); 3.05-3.20 (m, 1H, N—CHₐH_{b}); 3.86 (s, 2H, N—CH₂); 3.90-4.03 (m, 1H, N—CHₐH_{b}); 4.06 (q, J 6.9 Hz, 2H, O—CH₂); 4.24-4.38 (m, 1H, N—CHₐH_{b}); 7.15-7.22 (m, 1H, Ar); 7.29-7.39 (m, 2H, 2Ar); 7.50-7.60 (m, 2H, 2Ar).

M/Z (M[³⁵Cl]+H)⁺: 460.3

### Example 33: 1-(1-(4-chloro-3-fluorophenyl)-3,3-dimethyl-2,3-dihydro-1H-pyrrolo[3,2-b]pyridine-5-carbonyl)piperidine-4-carboxylic acid

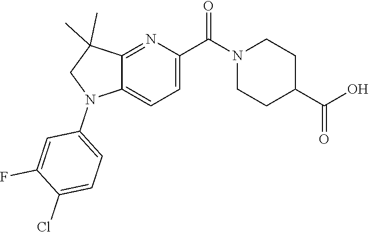 [SMILES: CC1(C)CN(c2ccc(Cl)c(F)c2)c2ccc(C(=O)N3CCC(C(=O)O)CC3)nc21]

Example 33 was prepared according to general procedure (VII) starting from Example 32 (36 mg). The crude was purified by preparative HPLC (H₂O+0.1% HCOOH/MeCN+0.1% HCOOH 60:40 to 20:80) and freeze dried to obtain Example 33 (25 mg, 74%) as a white solid.

¹H-NMR (DMSO-d₆, 300 MHz) δ: 1.33 (s, 6H, 2*CH₃); 1.42-1.66 (m, 2H, CH₂); 1.78-2.00 (m, 2H, CH₂); 2.50-2.60 (m, 1H, CH); 2.88-3.04 (m, 1H, N—CHₐH_{b}); 3.05-3.20 (m, 1H, N—CHₐH_{b}); 3.86 (s, 2H, N—CH₂); 3.90-4.05 (m, 1H, N—CHₐH_{b}); 4.24-4.38 (m, 1H, N—CHₐH_{b}); 7.15-7.22 (m, 1H, Ar); 7.29-7.39 (m, 2H, 2Ar); 7.50-7.60 (m, 2H, 2Ar); 12.32 (bs, 1H, COOH).

M/Z (M[³⁵Cl]+H)⁺: 432.3

### Example 34: methyl 2-(1-(1-(4-chloro-3-fluorophenyl)-3,3-dimethyl-2,3-dihydro-1H-pyrrolo[3,2-b]pyridine-5-carbonyl)piperidin-4-yl)acetate

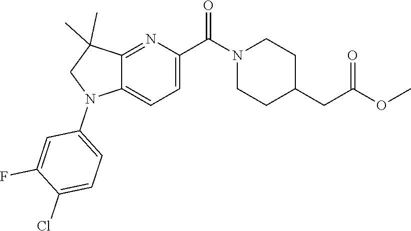 [SMILES: COC(=O)CC1CCN(C(=O)c2ccc3c(n2)C(C)(C)CN3c2ccc(Cl)c(F)c2)CC1]

Example 34 was prepared according to general procedure (VIIIa) starting from Compound 68 (70 mg), methyl 2-(piperidin-4-yl)acetate (1.0 eq) and N,N-diisopropylethylamine (4.0 eq.) in DCM. The crude was purified by flash chromatography (Interchim®50 μm, CyHex 100% to CyHex/EtOAc 20:80) to obtain Example 34 (68 mg) as a white solid.

¹H-NMR (CDCl₃, 300 MHz) δ: 1.29-1.45 (m, 10H, 2CH₃+2N—CH₂—CHₐH_{b}); 1.68-1.87 (m, 2H, 2N—CH₂—CHₐH_{b}); 2.04-2.13 (m, 1H, CH); 2.26-2.34 (m, 2H, CH₂CO); 2.76-2.88 (m, 1H, N—CHₐH_{b}); 3.02-3.12 (m, 1H, N—CHₐH_{b}); 3.68 (s, 3H, OCH₃); 3.76 (s, 2H, N—CH₂); 4.20-4.28 (m, 1H, N—CHₐH_{b}); 4.65-4.73 (m, 1H, N—CHₐH_{b}); 6.91-7.01 (m, 2H, 2Ar); 7.32-7.38 (m, 2H, 2Ar); 7.47 (d, J 8.5 Hz, 1H, Ar).

M/Z (M[³⁵Cl]+H)⁺: 460.3

### Example 35: 2-(1-(1-(4-chloro-3-fluorophenyl)-3,3-dimethyl-2,3-dihydro-1H-pyrrolo[3,2-b]pyridine-5-carbonyl)piperidin-4-yl)acetic acid

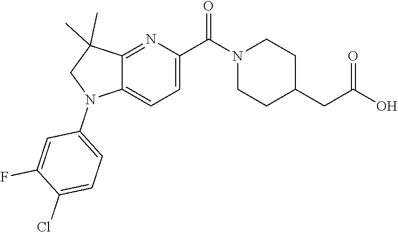 [SMILES: CC1(C)CN(c2ccc(Cl)c(F)c2)c2ccc(C(=O)N3CCC(CC(=O)O)CC3)nc21]

Example 35 was prepared according to general procedure (VII) starting from Example 34 (72 mg). The crude was purified by preparative HPLC (Column B, H₂O+0.1% HCOOH/MeCN+0.1% HCOOH 20:80 to 0:100) and freeze dried to obtain Example 35 (48 mg, 69%) as a white solid.

¹H-NMR (DMSO-d₆, 300 MHz) δ: 1.13-1.23 (m, 2H, CH₂); 1.31 (s, 6H, (CH₃)₂); 1.62-1.76 (m, 2H, CH₂); 1.89-1.99 (m, 1H, CH₂); 2.15-2.20 (m, 2H, CH₂); 2.77-2.83 (m, 1H, CH); 2.92-3.20 (m, 1H, CH); 3.85 (s, 2H, (CH₂); 3.97-4.02 (m, 1H, CH); 4.40-4.45 (m, 1H, CH); 7.15-7.18 (m, 1H, Ar); 7.29-7.34 (m, 2H, 2Ar); 7.50-7.56 (m, 2H, 2Ar); 12.13 (bs, 1H, CO₂H).

M/Z (M[³⁵Cl]+H)⁺: 446.3

### Example 36: methyl 2-((3R,4S)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetate

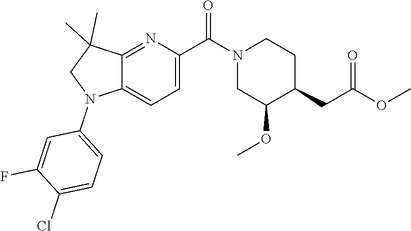 [SMILES: COC(=O)C[C@@H]1CCN(C(=O)c2ccc3c(n2)C(C)(C)CN3c2ccc(Cl)c(F)c2)C[C@@H]1OC]

Example 36 was prepared according to general procedure (VIIIa) starting from Compound 68 (70 mg), methyl 2-((3R,4S)-3-methoxypiperidin-4-yl)acetate (1.0 eq) and N,N-diisopropylethylamine (4.0 eq.) in DCM. The crude was purified by flash chromatography (Interchim®50 μm, CyHex 100% to CyHex/EtOAc 20:80) to obtain Example 36 (80 mg) as a white solid.

¹H-NMR (CDCl₃, 300 MHz) δ: 1.40-1.58 (m, 7H, (CH₃)₂+N—CH₂CHₐH_{b}); 1.75-1.78 (m, 1H, N—CH₂CHₐH_{b}); 2.20-2.31 (m, 2H, CH₂—CO); 2.51-2.56 (m, 1H, CH); 2.89-2.97 (m, 2H, 2N—CHₐH_{b}); 3.20-3.25 (m, 1H, CH—OCH₃); 3.38-3.42 (m, 2H, N—CH₂); 3.62-3.65 (m, 3H, O—CH₃); 3.67-3.72 (m, 3H, COOCH₃); 4.14-4.22 (m, 0.5H, one rotamer of N—CHₐH_{b}); 4.49-4.60 (m, 1H, N—CHₐH_{b}); 4.82-4.94 (m, 0.5H, one rotamer of N—CHₐH_{b}); 6.91-7.00 (m, 2H, 2Ar); 7.33-7.39 (m, 2H, 2Ar); 7.50-7.58 (m, 1H, Ar).

M/Z (M[³⁵Cl]+H)⁺: 490.3

### Example 37: 2-((3R,4S)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetic acid

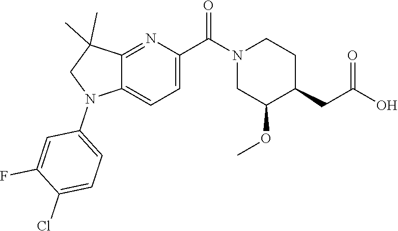 [SMILES: CO[C@H]1CN(C(=O)c2ccc3c(n2)C(C)(C)CN3c2ccc(Cl)c(F)c2)CC[C@H]1CC(=O)O]

Example 37 was prepared according to general procedure (VII) starting from Example 36 (80 mg). The crude was purified by preparative HPLC (Column B, H₂O+0.1% HCOOH/MeCN+0.1% HCOOH 20:80 to 0:100) and freeze dried to obtain Example 37 (55 mg, 53% over 2 steps) as a yellow solid.

¹H-NMR (DMSO-d₆, 300 MHz) δ: 1.31-1.36 (m, 6H, (CH₃)₂); 1.43-1.60 (m, 2H, CH₂); 2.03-2.36 (m, 2H, CH₂); 2.80-2.89 (m, 3H, CH₃); 3.13-3.28 (m, 3H, CH₃); 3.38-3.42 (m, 1H, CH); 3.80-3.86 (m, 2H, CH₂); 3.88-4.05 (m, 0.5H, one rotamer of CHₐH_{b}); 4.25-4.34 (m, 0.5H, other rotamer of CHₐH_{b}); 4.37-4.45 (m, 0.5H, one rotamer of CHₐH_{b}); 4.63-4.70 (m, 0.5H, one rotamer of CHₐH_{b}); 7.16 (dd, J 9.0, 2.1 Hz, 1H, Ar); 7.28-7.38 (m, 2H, 2Ar); 7.51-7.59 (m, 2H, 2 Ar). COOH was not observed.

M/Z (M[³⁵Cl]+H)⁺: 476.3.

### Example 38: ethyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylpyrimidine-5-carboxylate

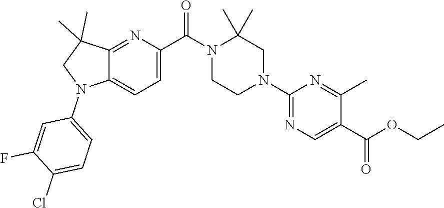 [SMILES: CCOC(=O)c1cnc(N2CCN(C(=O)c3ccc4c(n3)C(C)(C)CN4c3ccc(Cl)c(F)c3)C(C)(C)C2)nc1C]

Example 38 was prepared according to general procedure (VIIIb) starting from Compound 68 (80 mg), Compound 2 (1.2 eq.) and triethylamine (3 eq.) in THF. The crude was purified by flash chromatography (Merck 60®, CyHex 100% to CyHex/EtOAc 70:30) to obtain Example 38 (103 mg, 71%) as a clear oil.

¹H-NMR (CDCl₃, 300 MHz) δ: 1.36 (t, J 7.2 Hz, 3H, OCH₂—CH₃); 1.42 (s, 6H, 2*CH₃); 1.63 (s, 6H, 2*CH₃); 2.68 (s, 3H, CH₃), 3.77 (s, 2H, N—CH₂); 3.90-4.08 (m, 6H, 3N—CH₂); 4.31 (q, J 7.2 Hz, 2H, O—CH₂); 6.84-7.00 (m, 2H, 2Ar); 7.31-7.38 (m, 2H, 2Ar); 7.49 (d, J 8.4 Hz, Ar); 8.86 (s, 1H, Ar).

M/Z (M[³⁵Cl]+H)⁺: 581.4

### Example 39: 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylpyrimidine-5-carboxylic acid hydrochloride

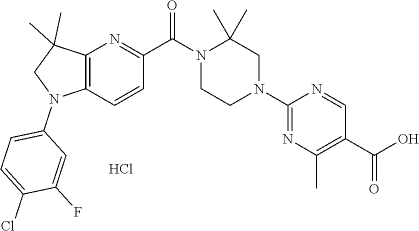 [SMILES: Cc1nc(N2CCN(C(=O)c3ccc4c(n3)C(C)(C)CN4c3ccc(Cl)c(F)c3)C(C)(C)C2)ncc1C(=O)O ; Cl]

Example 39 was prepared according to general procedure (VII) starting from Example 38 (103 mg). The crude was purified by preparative HPLC (Column B, H₂O+0.1% HCOOH/MeCN+0.1% HCOOH 80:20 to 0:100) then freeze-dried with aqueous HCl to obtain Example 39 (45 mg, 43%) as a yellow solid.

¹H-NMR (DMSO-d₆, 300 MHz) δ: 1.34 (s, 6H, 2*CH₃); 1.49 (s, 6H, 2*CH₃); 2.58 (s, 3H, CH₃), 3.75-3.83 (m, 2H, N—CH₂—CH₂—N); 3.84 (s, 2H, N—CH₂—C—N); 4.05 (s, 2H, N—CH₂); 7.17 (dd, J 8.7, 2.1 Hz, 1H, Ar); 7.27-7.37 (m, 2H, 2 Ar), 7.48-7.58 (m, 2H, 2Ar); 8.74 (s, 1H, Ar). One CH₂ signal, HCl and COOH were not observed.

M/Z (M[³⁵Cl]+H)⁺: 553.4.

### Example 40: ethyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyrimidine-5-carboxylate

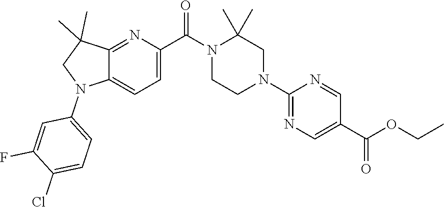 [SMILES: CCOC(=O)c1cnc(N2CCN(C(=O)c3ccc4c(n3)C(C)(C)CN4c3ccc(Cl)c(F)c3)C(C)(C)C2)nc1]

Example 40 was prepared according to general procedure (VIIIb) starting from Compound 68 (80 mg), Compound 4 (1.2 eq.) and triethylamine (3 eq.) in THF. The crude was purified by flash chromatography (Merck 60®, CyHex 100% to CyHex/EtOAc 70:30) to obtain Example 40 (98 mg, 69%) as a white solid.

¹H-NMR (CDCl₃, 300 MHz) δ: 1.37 (t, J 7.2 Hz, 3H, OCH₂—CH₃); 1.42 (s, 6H, 2*CH₃); 1.64 (s, 6H, 2*CH₃); 3.77 (s, 2H, N—CH₂); 3.90-3.99 (m, 4H, 2N—CH₂); 4.07 (s, 2H, N—CH₂); 4.35 (q, J 7.2 Hz, 2H, O—CH₂); 6.86-7.02 (m, 2H, 2Ar); 7.33-7.39 (m, 2H, 2Ar); 7.51 (d, J 8.4 Hz, Ar); 8.90 (s, 2H, 2Ar).

M/Z (M[³⁵Cl]+H)⁺: 567.4

### Example 41: 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-pyrimidine-5-carboxylic acid hydrochloride

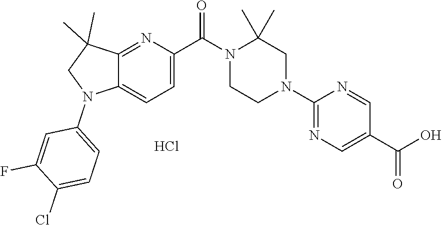 [SMILES: CC1(C)CN(c2ccc(Cl)c(F)c2)c2ccc(C(=O)N3CCN(c4ncc(C(=O)O)cn4)CC3(C)C)nc21 ; Cl]

Example 41 was prepared according to general procedure (VII) starting from Example 40 (103 mg). The crude was purified by preparative HPLC (Column B, H₂O+0.1% HCOOH/MeCN+0.1% HCOOH 80:20 to 0:100) then freeze-dried with aqueous HCl to obtain Example 41 (39 mg, 40%) as a yellow solid.

¹H-NMR (DMSO-d₆, 300 MHz) δ: 1.35 (s, 6H, 2*CH₃); 1.51 (s, 6H, 2*CH₃); 3.75-3.80 (m, 2H, N—CH₂—CH₂—N); 3.82- 3.88 (m, 4H, N—CH₂—CH₂—N+N—CH₂—C—N); 4.05 (s, 2H, N—CH₂) 7.18 (dd, J 9.0, 2.1 Hz, 1H, Ar); 7.27-7.37 (m, 2H, 2 Ar), 7.50-7.60 (m, 2H, 2Ar); 8.77-8.84 (m, 2H, 2Ar). HCl and COOH were not observed.

M/Z (M[³⁵Cl]+H)⁺: 539.4.

### Example 42: ethyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)thiazole-4-carboxylate

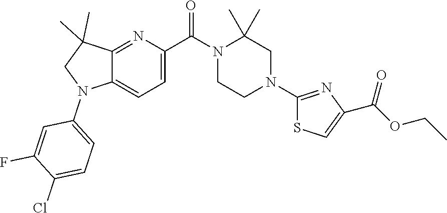 [SMILES: CCOC(=O)c1csc(N2CCN(C(=O)c3ccc4c(n3)C(C)(C)CN4c3ccc(Cl)c(F)c3)C(C)(C)C2)n1]

Example 42 was prepared according to general procedure (VIIIa) starting from Compound 68 (62 mg), Compound 6 (1.2 eq.) and N,N-diisopropylethylamine (3 eq.) in DCM. The crude was purified by flash chromatography (Interchim® 50 μm, CyHex 100% to CyHex/EtOAc 15:85) to obtain Example 42 (86 mg, 78%) as a white solid.

¹H-NMR (DMSO-d₆, 300 MHz) δ: 1.27 (t, J 7.2 Hz, 3H, OCH₂—CH₃); 1.35 (s, 6H, 2*CH₃); 1.54 (s, 6H, 2*CH₃); 3.53-3.60 (m, 2H, N—CH₂—CH₂—N); 3.72 (s, 2H, N—CH₂); 3.78-3.86 (m, 2H, N—CH₂—CH₂—N); 3.87 (s, 2H, N—CH₂); 4.23 (q, J 7.2 Hz, 2H, O—CH₂); 7.19 (dd, J 8.7, 1.9 Hz, 1H, Ar); 7.30 (d, J 8.7 Hz, 1H, Ar); 7.33 (dd, J 11.9, 2.6 Hz, 1H, Ar); 7.51-7.60 (m, 2H, 2Ar); 7.65 (s, 1H, Ar).

M/Z (M[³⁵Cl]+H)⁺: 572.4

### Example 43: 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)thiazole-4-carboxylic acid

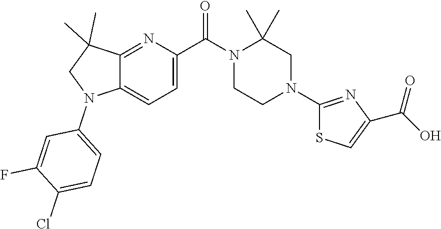 [SMILES: CC1(C)CN(c2ccc(Cl)c(F)c2)c2ccc(C(=O)N3CCN(c4nc(C(=O)O)cs4)CC3(C)C)nc21]

Example 43 was prepared according to general procedure (VII) starting from Example 42 (83 mg). The crude was purified by preparative HPLC (H₂O+0.1% HCOOH/MeCN+0.1% HCOOH 50:50 to 10:90) then freeze-dried to obtain Example 43 (50 mg, 63%) as a white solid.

¹H-NMR (DMSO-d₆, 300 MHz) δ: 1.35 (s, 6H, 2*CH₃); 1.53 (s, 6H, 2*CH₃); 3.52-3.60 (m, 2H, N—CH₂—CH₂—N); 3.72 (s, 2H, N—CH₂); 3.78-3.86 (m, 2H, N—CH₂—CH₂—N); 3.87 (s, 2H, N—CH₂); 7.19 (dd, J 8.7, 1.9 Hz, 1H, Ar); 7.30 (d, J 8.7 Hz, 1H, Ar); 7.33 (dd, J 11.9, 2.6 Hz, 1H, Ar); 7.51-7.60 (m, 3H, 3Ar); 12.62 (bs, 1H, COOH).

M/Z (M[³⁵Cl]+H)⁺: 544.5.

### Example 44: methyl 6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-2,4-dimethylnicotinate

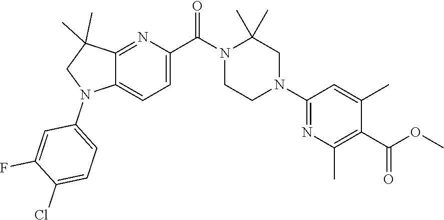 [SMILES: COC(=O)c1c(C)cc(N2CCN(C(=O)c3ccc4c(n3)C(C)(C)CN4c3ccc(Cl)c(F)c3)C(C)(C)C2)nc1C]

Example 44 was prepared according to general procedure (XIV) starting from Compound 71 (600 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 44 (755 mg, 68%) as a yellow solid.

¹H-NMR (CDCl₃, 300 MHz) δ: 1.42 (s, 6H, C(CH₃)₂); 1.60 (s, 6H, C(CH₃)₂); 2.32 (s, 3H, ArCH₃); 2.47 (s, 3H, ArCH₃); 3.58-3.64 (m, 2H, N—CH₂—CH₂); 3.76 (s, 2H, N—CH₂); 3.85 (s, 3H, OCH₃); 3.93 (s, 2H, N—CH₂); 3.95-4.02 (m, 2H, N—CH₂—CH₂); 6.10 (s, 1H, Ar); 6.83-7.02 (m, 2H, 2Ar); 7.31-7.36 (m, 2H, 2Ar); 7.48 (d, J 8.4 Hz, 1H, Ar). M/Z (M[³⁵Cl]+H⁺): 580.4.

### Example 45: 6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-2,4-dimethylnicotinic acid

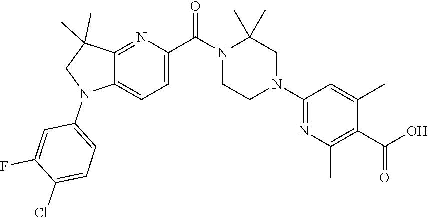 [SMILES: Cc1cc(N2CCN(C(=O)c3ccc4c(n3)C(C)(C)CN4c3ccc(Cl)c(F)c3)C(C)(C)C2)nc(C)c1C(=O)O]

Example 45 was prepared according to general procedure (XI) starting from Example 44 (275 mg). The crude was purified by flash chromatography (Interchim® 50 μm, DCM 100% to DCM/MeOH 90:10), then freeze-dried with water/Ethanol (9:1) to obtain Example 45 (160 mg, 60%) as a white solid.

¹H-NMR (DMSO-d₆, 300 MHz) δ: 1.36 (s, 6H, C(CH₃)₂); 1.49 (s, 6H, C(CH₃)₂); 2.27 (s, 3H, ArCH₃); 2.38 (s, 3H, ArCH₃); 3.53-3.56 (m, 2H, CH₂); 3.80-3.88 (m, 6H, 3*CH₂); 6.34 (s, 1H, Ar); 7.18 (dd, J 1.7, 7.5 Hz, 1H, Ar); 7.30-7.36 (m, 2H, 2*Ar); 7.52-7.58 (m, 2H, 2*Ar); 12.65 (bs, 1H, COOH).

M/Z (M[³⁵Cl]+H)⁺: 566.4

### Example 46: methyl (R)-3-(1-(4-chloro-3-fluorophenyl)-N,3,3-trimethyl-2,3-dihydro-1H-pyrrolo[3,2-b]pyridine-5-carboxamido)butanoate

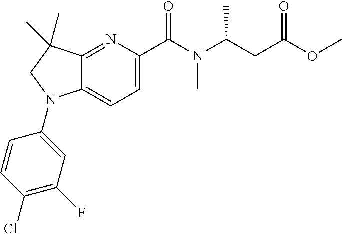 [SMILES: COC(=O)C[C@@H](C)N(C)C(=O)c1ccc2c(n1)C(C)(C)CN2c1ccc(Cl)c(F)c1]

Example 46 was prepared according to general procedure (VIIIa) starting from Compound 68 (50 mg), methyl (R)-3-(methylamino)butanoate hydrochloride (32 mg, 1.2 eq) and N,N-diisopropylethylamine (4.0 eq.) in DCM. The crude was purified by flash chromatography (Interchim®50 μm, CyHex 100% to CyHex/EtOAc 40:60) to obtain Example 46 (56 mg, 83%) as a white solid.

¹H-NMR (DMSO-d₆, 300 MHz) δ: 1.16-1.41 (m, 9H, 3*CH₃); 2.55-2.78 (m, 2H, CH₂—COOH); 2.80 (s, 2.3H, one rotamer of N—CH₃); 2.89 (s, 0.7H, one rotamer of N—CH₃); 3.52-3.61 (m, 3H, OCH₃); 3.80-3.90 (m, 2H, N—CH₂); 4.31-4.49 (m, 0.8H, one rotamer of N—CH); 4.82-4.95 (m, 0.2H, one rotamer of N—CH); 7.15-7.21 (m, 1H, Ar); 7.28-7.36 (m, 2H, 2Ar); 7.50-7.60 (m, 2H, 2Ar).

M/Z (M[³⁵Cl]+H)⁺: 434.2

### Example 47: (R)-3-(1-(4-chloro-3-fluorophenyl)-N,3,3-trimethyl-2,3-dihydro-1H-pyrrolo[3,2-b]pyridine-5-carboxamido)butanoic acid

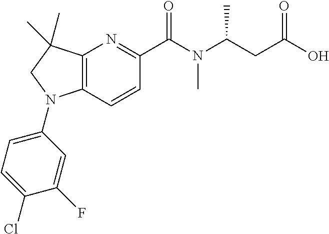 [SMILES: C[C@H](CC(=O)O)N(C)C(=O)c1ccc2c(n1)C(C)(C)CN2c1ccc(Cl)c(F)c1]

Example 47 was prepared according to general procedure (VII) starting from Example 46 (160 mg). The crude was purified by trituration in MeCN (2 mL) then freeze dried to obtain Example 47 (33 mg, 57%) as a white solid.

¹H-NMR (DMSO-d₆, 300 MHz) δ: 1.16-1.38 (m, 9H, 3*CH₃); 2.55-2.75 (m, 2H, CH₂—COOH); 2.81 (s, 2.3H, one rotamer of N—CH₃); 2.90 (s, 0.7H, one rotamer of N—CH₃); 3.80-3.90 (m, 2H, N—CH₂); 4.32-4.45 (m, 0.8H, one rotamer of N—CH); 4.82-4.95 (m, 0.2H, one rotamer of N—CH); 7.15-7.21 (m, 1H, Ar); 7.28-7.32 (m, 1H, Ar); 7.32-7.36 (m, 1H, Ar); 7.50-7.60 (m, 2H, 2Ar); 12.20 (m, 1H, COOH).

M/Z (M[³⁵Cl]+H)⁺: 420.2.

### Example 48: N-(1-carbamoylcyclopropyl)-1-(4-chloro-3-fluorophenyl)-N,3,3-trimethyl-2,3-dihydro-1H-pyrrolo[3,2-b]pyridine-5-carboxamide

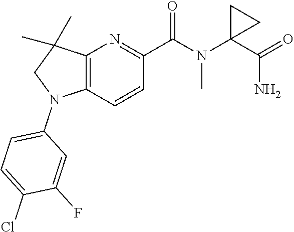 [SMILES: CN(C(=O)c1ccc2c(n1)C(C)(C)CN2c1ccc(Cl)c(F)c1)C1(C(N)=O)CC1]

Example 48 was prepared according to general procedure (VIIIa) starting from Compound 68 (70 mg), Compound 13 (33 mg) and N,N-diisopropylethylamine (5.0 eq.) in DCM. The crude was purified by flash chromatography (Merck 60®, CyHex 100% to CyHex/EtOAc 0:100 to EtOAc/MeOH 80:20) then further purified by preparative HPLC (Column B, H₂O+0.1% HCOOH/MeCN+0.1% HCOOH 80:20 to 0:100) and freeze dried to obtain Example 48 (42 mg, 46%) as a white solid.

¹H-NMR (DMSO-d₆, 300 MHz) δ: 0.43-0.60 (m, 1H, CH₂); 0.96-1.05 (m, 1H, CH₂); 1.11-1.20 (m, 2H, CH₂); 1.31-1.39 (m, 6H, 2CH₃); 2.95-3.16 (m, 3H, CH₃); 3.77-3.90 (m, 2H, CH₂); 7.15-7.23 (m, 2H, CO—NH₂); 7.31 (dd, J 12.1, 2.3 Hz, 1H, Ar); 7.47-7.60 (m, 4H, 4Ar).

M/Z (M[³⁵Cl]+H)⁺: 417.2

### Example 49: methyl 6-((2-(1-(4-chloro-3-fluorophenyl)-N,3,3-trimethyl-2,3-dihydro-1H-pyrrolo[3,2-b]pyridine-5-carboxamido)ethyl)(methyl)amino)-2,4-dimethylnicotinate

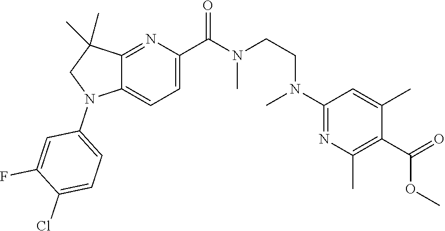 [SMILES: COC(=O)c1c(C)cc(N(C)CCN(C)C(=O)c2ccc3c(n2)C(C)(C)CN3c2ccc(Cl)c(F)c2)nc1C]

Example 49 was prepared according to general procedure (VIIIa) starting from Compound 68 (62 mg), Compound 8 (1.2 eq.) and N,N-diisopropylethylamine (3 eq.) in DCM. The crude was purified by flash chromatography (Interchim® 50 μm, CyHex 100% to CyHex/EtOAc 20:80) to obtain Example 49 (87 mg) as a white solid.

¹H-NMR (DMSO-d₆, 300 MHz) δ: 1.24-1.32 (m, 6H, 2*CH₃); 2.05 (s, 3H, CH₃); 2.18 (s, 3H, CH₃); 2.80 (s, 3H, N—CH₃); 3.02 (s, 3H, N—CH₃); 3.04-3.08 (m, 2H, N—CH₂); 3.63-3.67 (m, 2H, N—CH₂); 3.76 (s, 2H, N—CH₃); 3.83 (s, 3H, O—CH₃); 6.02 (s, 1H, Ar); 7.02-7.40 (m, 4H, 4Ar); 7.50-7.60 (m, 1H, Ar).

M/Z (M[³⁵Cl]+H)⁺: 554.4

### Example 50: 6-((2-(1-(4-chloro-3-fluorophenyl)-N,3,3-trimethyl-2,3-dihydro-1H-pyrrolo[3,2-b]pyridine-5-carboxamido)ethyl)(methyl)amino)-2,4-dimethylnicotinic acid

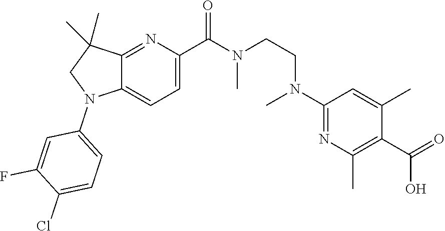 [SMILES: Cc1cc(N(C)CCN(C)C(=O)c2ccc3c(n2)C(C)(C)CN3c2ccc(Cl)c(F)c2)nc(C)c1C(=O)O]

Example 50 was prepared according to general procedure (XI) starting from Example 49 (193 μmol). The crude was purified by preparative HPLC (H₂O+0.1% HCOOH/MeCN+0.1% HCOOH 70:30 to 30:70). The white solid was dissolved in 2 mL of HCl 1.25M in MeOH then concentrated to dryness. The yellow residue was dissolved in 1 mL of DCM then poured into 15 mL of Et₂O. The precipitate was filtered, dissolved in MeCN then freeze-dried with water to obtain Example 50 (23 mg, 21% over 2 steps) as a white solid.

¹H-NMR (DMSO-d₆, 300 MHz) δ: 1.24-1.32 (m, 6H, 2*CH₃); 2.14 (s, 2H, one rotamer of CH₃); 2.33 (s, 3H, CH₃); 2.40-2.50 (m, 1H, one rotamer of CH₃); 2.80-2.95 (m, 2H, N—CH₂); 3.04 (s, 2H, N—CH₂); 3.12-3.17 (m, 1H, N—CHaHb); 3.17-3.24 (m, 1H, N—CHaHb); 3.77 (m, 3H, N—CH₃); 3.82-3.88 (m, 1H, one rotamer of N—CH₃); 3.88-3.98 (m, 2H, one rotamer of N—CH₃); 6.10-6.50 (m, 0.6H, one rotamer of Ar); 6.70-6.95 (m, 0.4H, one rotamer of Ar); 7.05-7.40 (m, 4H, 4Ar); 7.50-7.60 (m, 1H, Ar). COOH was not observed.

M/Z (M[³⁵Cl]+H)⁺: 540.5.

### Example 51: 1-(4-chloro-3-fluorophenyl)-N-(1-hydroxy-2-methylpropan-2-yl)-3,3-dimethyl-2,3-dihydro-1H-pyrrolo[3,2-b]pyridine-5-carboxamide

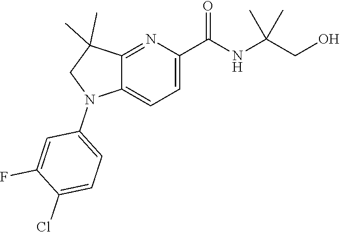 [SMILES: CC(C)(CO)NC(=O)c1ccc2c(n1)C(C)(C)CN2c1ccc(Cl)c(F)c1]

To a solution of Compound 65 (50 mg) in Chlorobenzene (2 mL) was added 2-amino-2-methylpropan-1-ol (1.5 eq) and zinc chloride (0.1 eq). The mixture was heated at 140° C. for 18 hours then concentrated under reduced pressure. The crude was purified by flash chromatography (Merck 60®, CyHex 100% to CyHex/EtOAc 0:100) to obtain Example 51 (37 mg, 57%) as a white solid.

¹H-NMR (DMSO-d₆, 300 MHz) δ: 1.34 and 1.35 (2s, 12H, 4×CH₃); 3.45 (d, J 5.4 Hz, 2H, CH₂OH); 3.88 (s, 2H, NCH₂); 5.15 (t, J 5.4 Hz, 1H, CH₂OH); 7.18-7.22 (m, 1H, Ar); 7.34 (dd, J 11.7, 2.7 Hz, 1H, Ar); 7.53-7.60 (m, 2H, 2 Ar); 7.75 (d, J 8.4 Hz, 1H, Ar); 8.06 (s, 1H, NH)

M/Z (M[³⁵Cl]+H)⁺: 392.3

### Example 52: 1-(4-chloro-3-fluorophenyl)-N-(2-hydroxy-2-methylpropyl)-3,3-dimethyl-2,3-dihydro-1H-pyrrolo[3,2-b]pyridine-5-carboxamide

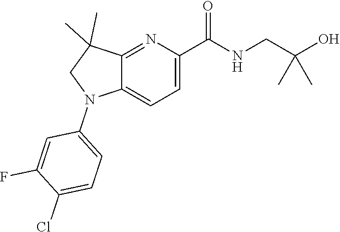 [SMILES: CC(C)(O)CNC(=O)c1ccc2c(n1)C(C)(C)CN2c1ccc(Cl)c(F)c1]

To a solution of Compound 65 (50 mg) in Chlorobenzene (2 mL) was added 1-amino-2-methylpropan-2-ol (24 μL, 1.5 eq) and zinc chloride (2.3 mg, 0.1 eq). The mixture was heated at 140° C. for 18 hours then concentrated under reduced pressure. The crude was purified by flash chromatography (Merck 60®, CyHex 100% to CyHex/EtOAc 0:100) to obtain Example 52 (45 mg, 69%) as a white solid.

¹H-NMR (DMSO-d₆, 300 MHz) δ: 1.11 (s, 6H, NHCH₂CMe₂); 1.37 (s, 6H, ArNCH₂CMe₂); 3.27 (d, J 6.3 Hz, 2H, CH₂NH); 3.89 (s, 2H, NCH₂); 4.70 (s, 1H, OH); 7.21 (dd, J 8.7, 2.1 Hz, 1H, Ar); 7.35 (dd, J 11.7, 2.7 Hz, 1H, Ar); 7.54-7.60 (m, 2H, 2Ar); 7.78 (d, J 8.1 Hz, 1H, Ar); 8.23 (t, J 6.3 Hz, 1H, NH).

M/Z (M[³⁵Cl]+H)⁺: 392.3

### Compound 72: 2-(3-bromo-6-chloropyridin-2-yl)-2-methylbutanenitrile

Compound 72 was prepared according to general procedure (I) starting from 3-bromo-6-chloro-2-fluoropyridine (5.00 g) and 2-methylbutanenitrile (2.40 mL). The crude was purified by flash chromatography (Merck 60®, CyHex 100% to CyHex/Et₂O 80:20) to obtain Compound 72 (2.44 g, 38%) as a clear oil.

M/Z (M[⁸¹Br³⁵Cl]+H)⁺: 274.9

### Compound 73: 2-(3-bromo-6-chloropyridin-2-yl)-2-methylbutanamide

Compound 73 (2.54 g, 98%) was obtained as a white solid from Compound 72 (2.44 g) according to general procedure (IIb).

M/Z (M[³⁵Cl][⁸¹Br]+H)⁺: 292.9.

### Compound 74: 5-chloro-3-ethyl-3-methyl-1,3-dihydro-2H-pyrrolo[3,2-b]pyridin-2-one

Compound 74 was prepared according to general procedure (III) starting from Compound 73 (2.50 g). The crude was purified by flash chromatography (Merck 60®, CyHex 100% to CyHex/EtOAc 0:100) to obtain Compound 74 (1.35 g, 75%) as a brown solid.

M/Z (M[³⁵Cl]+H)⁺: 210.9

### Compound 75: 5-chloro-3-ethyl-3-methyl-2,3-dihydro-1H-pyrrolo[3,2-b]pyridine

Compound 75 was prepared according to general procedure (IX) starting from Compound 74 (1.35 g). The crude was purified by flash chromatography (Merck 60®, CyHex 100% to CyHex/EtOAc 0:100 then DCM 100% to DCM/MeOH: 90/10) to obtain Compound 75 (864 mg, 68%) as a beige solid.

M/Z (M[³⁵Cl]+H)⁺197.1

### Compound 76: 3-ethyl-3-methyl-2,3-dihydro-1H-pyrrolo[3,2-b]pyridine-5-carbonitrile

Compound 76 was prepared according to general procedure (IV) starting from Compound 75 (629 mg). The crude was purified by flash chromatography (Merck 60®, CyHex 100% to CyHex/EtOAc 20:80) to obtain Compound 76 (210 mg, 35%) as a yellow oil.

M/Z (M+H)⁺: 188.1.

### Compound 77: 1-(4-chloro-3-fluorophenyl)-3-ethyl-3-methyl-2,3-dihydro-1H-pyrrolo[3,2-b]pyridine-5-carbonitrile

Compound 77 was prepared according to general procedure (X) starting from Compound 76 (210 mg) and 4-bromo-1-chloro-2-fluorobenzene (1.1 eq.). The crude was purified by flash chromatography (Merck 60®, CyHex 100% to CyHex/EtOAc 80:20) to obtain Compound 77 (250 mg) as a yellow oil.

M/Z (M[³⁵Cl]+H)+=316.2.

### Compound 78: 1-(4-chloro-3-fluorophenyl)-3-ethyl-3-methyl-2,3-dihydro-1H-pyrrolo[3,2-b]pyridine-5-carboxamide

Compound 78 was prepared according to general procedure (IIa) starting from Compound 77 (250 mg). The crude was purified by flash chromatography (Merck 60®, CyHex 100% to CyHex/EtOAc 50:50) to obtain Compound 78 (190 mg, 51% over 2 steps) as a white solid.

M/Z (M[³⁵Cl]+H)⁺: 334.2

### Compound 79: methyl 1-(4-chloro-3-fluorophenyl)-3-ethyl-3-methyl-2,3-dihydro-1H-pyrrolo[3,2-b]pyridine-5-carboxylate

Compound 79 was prepared according to general procedure (VI) starting from Compound 77 (190 mg). The crude was purified by flash chromatography (Merck 60®, CyHex 100% to CyHex/EtOAc 40:60) to obtain Compound 79 (140 mg, 70%) as a white solid.

M/Z (M[³⁵Cl]+H)⁺: 349.2

### Compound 80: 1-(4-chloro-3-fluorophenyl)-3-ethyl-3-methyl-2,3-dihydro-1H-pyrrolo[3,2-b]pyridine-5-carboxylic acid

Compound 80 was obtained (70 mg) as a yellow solid according to general procedure (VII) starting from Compound 79 (80 mg).

M/Z (M[³⁵Cl]+H)⁺: 335.0

### Example 53: 4-(1-(4-chloro-3-fluorophenyl)-3-ethyl-3-methyl-2,3-dihydro-1H-pyrrolo[3,2-b]pyridine-5-carbonyl)-3,3-dimethylpiperazin-2-one

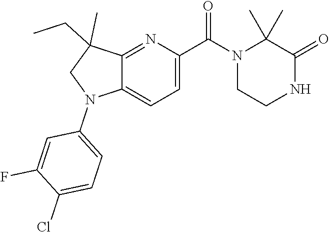 [SMILES: CCC1(C)CN(c2ccc(Cl)c(F)c2)c2ccc(C(=O)N3CCNC(=O)C3(C)C)nc21]

Example 53 was prepared according to general procedure (VIIIa) starting from Compound 80 (70 mg), 3,3-dimethylpiperazin-2-one (2.0 eq.) and N,N-diisopropylethylamine (2.0 eq.) in DCM. The crude was purified by flash chromatography (Merck 60®, CyHex 100% to CyHex/EtOAc 0:100) then further purified by preparative HPLC (Column B, H₂O+0.1% HCOOH/MeCN+0.1% HCOOH 80:20 to 0:100) and freeze dried to obtain Example 53 (50 mg, 49% over 2 steps) as a white solid.

¹H-NMR (DMSO-d₆, 300 MHz) δ: 0.80 (t, J 7.5 Hz, 3H, CH3); 1.30 (s, 3H, CH3); 1.64-1.71 (m, 8H, CH2+2CH3); 3.49-3.54 (m, 2H, CH2); 3.79 (d, J 10.0 Hz, 1H, CH2); 3.94 (d, J 10.0 Hz, 1H, CH2); 7.20 (dd, J 8.8, 2.6 Hz, 1H, Ar); 7.34 (dd, J 11.9, 2.6 Hz, 1H, Ar); 7.41 (d, J 7.9 Hz, 1H, Ar); 7.51-7.58 (m, 2H, 2Ar); 8.05 (bs, 1H, CONHR); one CH₂ signal not observed.

M/Z (M[³⁵Cl]+H)⁺: 445.4.

Mp: 213-217° C.

### Example 54: N-(1-carbamoylcyclopropyl)-1-(4-chloro-3-fluorophenyl)-3-ethyl-3-methyl-2,3-dihydro-1H-pyrrolo[3,2-b]pyridine-5-carboxamide

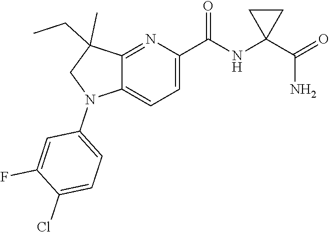 [SMILES: CCC1(C)CN(c2ccc(Cl)c(F)c2)c2ccc(C(=O)NC3(C(N)=O)CC3)nc21]

Example 54 was prepared according to general procedure (VIIIa) starting from Compound 80 (58 mg), 1-aminocyclopropane-1-carboxamide (1.5 eq.) and N,N-diisopropylethylamine (3.0 eq.) in DCM. The crude was purified by flash chromatography (Merck 60®, CyHex 100% to CyHex/EtOAc 0:100) then further purified by preparative HPLC (Column B, H₂O+0.1% HCOOH/MeCN+0.1% HCOOH 80:20 to 0:100) and freeze dried to obtain Example 54 (65 mg, 91% over 2 steps) as a yellow solid.

¹H-NMR (DMSO-d₆ 300 MHz) δ: 0.82 (t, J 7.6 Hz, 3H, CH₃); 1.00-1.04 (m, 2H, CH₂); 1.33-1.39 (m, 5H, CH₂+CH₃); 1.68-1.81 (m, 2H, CH₂); 3.81 (d, J 9.8 Hz, 1H, CH₂); 3.95 (d, J 9.8 Hz, 1H, CH₂); 7.00 (bs, 1H, CO—NH₂); 7.09 (bs, 1H, CO—NH₂); 7.21 (dd, J 8.8, 2.6 Hz, 1H, Ar); 7.35 (dd, J 11.8, 2.6 Hz, 1H, Ar); 7.52-7.58 (m, 2H, 2Ar); 7.76 (d, J 8.5 Hz, 1H, Ar); 8.74 (s, 1H, CO—NH). M/Z (M[³⁵Cl]+H)⁺: 417.3.

### Compound 81: 5′-chloro-1′,2,2′,3,5,6-hexahydrospiro[pyran-4,3′-pyrrolo[3,2-b]pyridine]

Compound 81 was prepared according to general procedure (IX) starting from Compound 16 (2.00 g). The crude was purified by flash chromatography (Merck 60®, CyHex 100% to CyHex/EtOAc 0:100) to obtain Compound 81 (1.32 g, 70%) as a brown solid.

M/Z (M[³⁵Cl]+H)⁺: 225.0

### Compound 82: 1′,2,2′,3,5,6-hexahydrospiro[pyran-4,3′-pyrrolo[3,2-b]pyridine]-5′-carbonitrile

Compound 82 was prepared according to general procedure (IV) starting from Compound 81 (500 mg). The reaction mixture was submitted to microwave irradiation at 150° C. for 1 hour. The crude was purified by flash chromatography (Merck 60®, CyHex 100% to CyHex/EtOAc 0:100) to obtain Compound 82 (300 mg, 63%) as a white solid.

M/Z (M+H)⁺: 216.2

### Compound 83: 1′-(4-chloro-3-fluorophenyl)-1′,2,2′,3,5,6-hexahydrospiro[pyran-4,3′-pyrrolo[3,2-b]pyridine]-5′-carbonitrile

Compound 83 was prepared according to general procedure (X) starting from Compound 82 (400 mg) and 4-bromo-1-chloro-2-fluorobenzene (1.0 eq.). The crude was purified by flash chromatography (Merck 60®, CyHex 100% to CyHex/EtOAc 50:50) to obtain Compound 83 (420 mg, 66%) as a white solid.

M/Z (M[³⁵Cl]+H)⁺: 344.2

### Compound 84: 1′-(4-chloro-3-fluorophenyl)-1′,2,2′,3,5,6-hexahydrospiro[pyran-4,3′-pyrrolo[3,2-b]pyridine]-5′-carboxamide

Compound 84 (414 mg, 96%) was obtained as a white solid from Compound 83 (410 mg) according to general procedure (IIb).

M/Z (M[³⁵Cl]+H)⁺: 362.2.

### Compound 85: methyl 1′-(4-chloro-3-fluorophenyl)-1′,2,2′,3,5,6-hexahydrospiro[pyran-4,3′-pyrrolo[3,2-b]pyridine]-5′-carboxylate

Compound 85 was prepared according to general procedure (VI) starting from Compound 84 (414 mg). The crude was purified by flash chromatography (Merck 60®, CyHex 100% to CyHex/EtOAc 40:60) to obtain Compound 85 (354 mg, 82%) as a white solid.

M/Z (M[³⁵Cl]+H)⁺: 377.2

### Compound 86: 1′-(4-chloro-3-fluorophenyl)-1′,2,2′,3,5,6-hexahydrospiro[pyran-4,3′-pyrrolo[3,2-b]pyridine]-5′-carboxylic acid

Compound 86 was obtained (314 mg, 93%) as a white solid according to general procedure (VII) starting from Compound 85 (350 mg).

M/Z (M[³⁵Cl]+H)⁺: 363.2

### Example 55: 4-(1′-(4-chloro-3-fluorophenyl)-1′,2,2′,3,5,6-hexahydrospiro[pyran-4,3′-pyrrolo[3,2-b]pyridine]-5′-carbonyl)-3,3-dimethylpiperazin-2-one

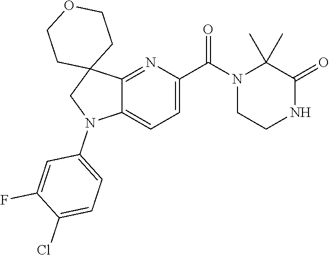 [SMILES: CC1(C)C(=O)NCCN1C(=O)c1ccc2c(n1)C1(CCOCC1)CN2c1ccc(Cl)c(F)c1]

Example 55 was prepared according to general procedure (VIIIa) starting from Compound 86 (80 mg), 3,3-dimethylpiperazin-2-one (2.0 eq.) and N,N-diisopropylethylamine (3.0 eq.) in DCM. The crude was purified by flash chromatography (Merck 60®, DCM 100% to DCM/MeOH 80:20) then further purified by preparative HPLC (Column B, H₂O+0.1% HCOOH/MeCN+0.1% HCOOH 95:05 to 0:100) and freeze dried to obtain Example 55 (43 mg, 41%) as a white solid.

¹H-NMR (CDCl₃, 300 MHz) δ: 1.60-1.64 (m, 2H, CH₂); 1.89 (s, 6H, (CH₃)₂); 2.13-2.23 (m, 2H, CH₂); 3.59-3.64 (m, 4H, 2CH₂); 3.76-3.80 (m, 2H, CH₂); 3.98 (s, 2H, CH₂); 4.07-4.13 (m, 2H, CH₂); 6.10 (bs, 1H, NH); 6.98-7.07 (m, 2H, 2 Ar); 7.37-7.44 (m, 2H, 2Ar); 7.63 (d, J 8.3 Hz, 1H, Ar).

M/Z (M[³⁵Cl]+H)⁺473.3.

Mp: 245-248° C.

### Example 56: 6-(4-(1′-(4-chloro-3-fluorophenyl)-1′,2,2′,3,5,6-hexahydrospiro[pyran-4,3′-pyrrolo[3,2-b]pyridine]-5′-carbonyl)-3,3-dimethylpiperazin-1-yl)-2,4-dimethylnicotinic acid

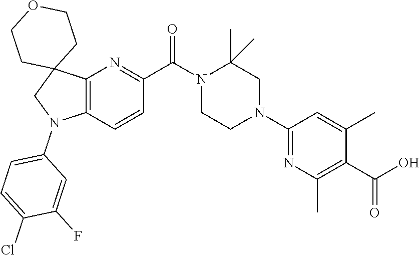 [SMILES: Cc1cc(N2CCN(C(=O)c3ccc4c(n3)C3(CCOCC3)CN4c3ccc(Cl)c(F)c3)C(C)(C)C2)nc(C)c1C(=O)O]

To a solution of Compound 86 (50 mg) in DMF (5 mL) was added 1,8-Diazabicyclo[5.4.0]undec-7-ene (62 μL) and benzotriazol-1-yloxytris(dimethylamino)phosphonium hexafluorophosphate (61 mg). The mixture was stirred at 25° C. for 5 minutes then a suspension of Compound 11 (1.0 eq) in DMF (5 mL) was added and the mixture was stirred at 25° C. for 72 hours, then at 80° C. for 8 hours. The reaction mixture was filtered and the filtrate was hydrolysed (100 mL) and extracted with Et₂O (3*50 mL). The organic layer was washed with brine, dried over MgSO₄ and concentrated under reduced pressure. The crude was purified by flash chromatography (Merck 60®, DCM 100% to DCM/MeOH 80:20) then further purified by preparative HPLC (Column B, H₂O+0.1% HCOOH/MeCN+0.1% HCOOH 80:20 to 0:100) and freeze dried to obtain Example 56 (30 mg, 36%) as a white solid.

¹H-NMR (DMSO-d₆, 300 MHz) δ: 1.48 (s, 6H, (CH₃)₂); 1.60-1.64 (m, 2H, CH₂); 1.94-2.04 (m, 2H, CH₂); 2.24 (s, 3H, CH₃); 2.35 (s, 3H, CH₃); 3.54-3.61 (m, 4H (CH₂)₂); 3.81-3.94 (m, 6H, (CH₂)₃); 4.08 (s, 2H, CH₂); 6.30 (s, 1H, Ar); 7.24 (dd, J 8.7, 2.1 Hz, 1H, Ar); 7.35-7.40 (m, 2H, 2Ar); 7.52-7.60 (m, 2H, 2Ar).

M/Z (M[³⁵Cl]+H)⁺: 608.5.

### Compound 87: 1-(3-bromo-6-chloropyridin-2-yl)cyclopropane-1-carbonitrile

Compound 87 was prepared according to general procedure (I) starting from 3-bromo-6-chloro-2-fluoropyridine (1.00 g) and cyclopropanecarbonitrile (350 μL). The crude was purified by flash chromatography (Merck 60®, CyHex 100% to CyHex/EtOAc 80:20) to obtain Compound 87 (370 mg, 30%) as a white solid.

M/Z (M[³⁵Cl][⁸¹Br]+H)⁺: 259.0

### Compound 88: 1-(3-bromo-6-chloropyridin-2-yl)cyclopropane-1-carboxamide

Compound 88 was prepared according to general procedure (IIa) starting from Compound 87 (680 mg). The crude was purified by flash chromatography (Merck 60®, CyHex 100% to CyHex/EtOAc 0:100) to obtain Compound 88 (740 mg) as a white solid.

M/Z (M[³⁵Cl⁸¹Br]+H)⁺: 277.1

### Compound 89: 5′-chlorospiro[cyclopropane-1,3′-pyrrolo[3,2-b]pyridin]-2′(1′H)-one

Compound 89 was prepared according to general procedure (III) starting from Compound 88 (740 mg). The crude was purified by flash chromatography (Merck 60®, CyHex 100% to CyHex/EtOAc 0:100) to obtain Compound 89 (400 mg) as a brown solid.

M/Z (M[³⁵Cl]+H)⁺: 195.0

### Compound 90: 5′-chloro-1′,2′-dihydrospiro[cyclopropane-1,3′-pyrrolo[3,2-b]pyridine]

Compound 90 was prepared according to general procedure (IX) starting from Compound 89 (1.60 g). The crude was purified by flash chromatography (Merck 60®, CyHex 100% to EtOAc 100% to EtOAc/MeOH 80:20) to obtain Compound 90 (210 mg 44% over 3 steps) as a brown solid.

M/Z (M[³⁵Cl]+H)⁺: 181.0

### Compound 91: 1′,2′-dihydrospiro[cyclopropane-1,3′-pyrrolo[3,2-b]pyridine]-5′-carbonitrile

Compound 91 was prepared according to general procedure (IV) starting from Compound 90 (210 mg). The crude was purified by flash chromatography (Merck 60®, CyHex 100% to CyHex/EtOAc 0:100) to obtain Compound 91 (80 mg, 40%) as a yellow solid

M/Z (M+H)⁺: 172.0

### Compound 92: 1′-(4-chloro-3-fluorophenyl)-1′,2′-dihydrospiro[cyclopropane-1,3′-pyrrolo[3,2-b]pyridine]-5′-carbonitrile

Compound 92 was prepared according to general procedure (X) starting from Compound 91 (80 mg) and 4-bromo-1-chloro-2-fluorobenzene (1.1 eq.). The crude was purified by flash chromatography (Merck 60®, CyHex 100% to CyHex/EtOAc 20:80) to obtain Compound 92 (110 mg) as a yellow solid.

M/Z (M[³⁵Cl]+H)⁺: 300.1

### Compound 93: 1′-(4-chloro-3-fluorophenyl)-1′,2′-dihydrospiro[cyclopropane-1,3′-pyrrolo[3,2-b]pyridine]-5′-carboxamide

Compound 93 was prepared according to general procedure (IIa) starting from Compound 92 (110 mg). The crude was purified by flash chromatography (Merck 60®, CyHex 100% to CyHex/EtOAc 0:100) to obtain Compound 93 (60 mg, 40% over 2 steps) as a white solid.

M/Z (M[³⁵Cl]+H)⁺: 318.2

### Compound 94: methyl 1′-(4-chloro-3-fluorophenyl)-1′,2′-dihydrospiro[cyclopropane-1,3′-pyrrolo[3,2-b]pyridine]-5′-carboxylate

Compound 94 was prepared according to general procedure (VI) starting from Compound 93 (60 mg). The crude was purified by flash chromatography (Merck 60®, CyHex 100% to CyHex/EtOAc 40:60) to obtain Compound 94 (30 mg) as a white solid.

M/Z (M[³⁵Cl]+H)⁺: 333.2

### Compound 95: 1′-(4-chloro-3-fluorophenyl)-1′,2′-dihydrospiro[cyclopropane-1,3′-pyrrolo[3,2-b]pyridine]-5′-carboxylic acid

Compound 95 was obtained (30 mg) as a yellow solid according to general procedure (VII) starting from Compound 94 (29 mg).

M/Z (M[³⁵Cl]+H)⁺: 319.2

### Example 57: 4-(1′-(4-chloro-3-fluorophenyl)-1′,2′-dihydrospiro[cyclopropane-1,3′-pyrrolo[3,2-b]pyridine]-5′-carbonyl)-3,3-dimethylpiperazin-2-one

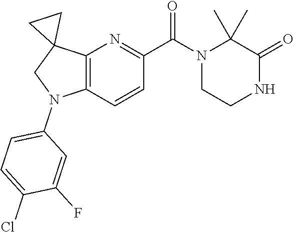 [SMILES: CC1(C)C(=O)NCCN1C(=O)c1ccc2c(n1)C1(CC1)CN2c1ccc(Cl)c(F)c1]

Example 57 was prepared according to general procedure (VIIIa) starting from Compound 95 (29 mg), 3,3-dimethylpiperazin-2-one (2.0 eq.) and N,N-diisopropylethylamine (2.0 eq.) in DCM. The crude was purified by flash chromatography (Merck 60®, CyHex 100% to CyHex/EtOAc 0:100) to obtain Example 57 (13 mg, 32% over 3 steps) as a white solid.

¹H-NMR (CDCl₃, 300 MHz) δ: 1.07-1.02 (m, 2H, CH₂); 1.30-1.35 (m, 2H, CH₂); 1.83 (s, 6H, 2CH₃); 3.51-3.55 (m, 2H, CH₂); 3.65-3.70 (m, 2H, CH₂); 4.10 (s, 2H, CH₂); 6.10 (bs, 1H, CO—NH₂); 6.93-7.03 (m, 2H, 2Ar); 7.28 (d, J 8.4 Hz, 1H, Ar); 7.37 (t, J 8.5 Hz, 1H, Ar); 7.51 (d, J 8.4 Hz, 1H, Ar).

M/Z (M[³⁵Cl]+H)⁺: 429.3.

Mp: 165-170° C.

### Compound 96: 3-(3-bromo-6-chloropyridin-2-yl)tetrahydrofuran-3-carbonitrile

Compound 96 was prepared according to general procedure (I) starting from 3-bromo-6-chloro-2-fluoropyridine (2.17 g) and tetrahydrofuran-3-carbonitrile (958 μL). The crude was purified by flash chromatography (Merck 60®, CyHex 100% to CyHex/Et₂O 40:60) to obtain Compound 96 (1.11 g, 38%) as a white solid.

M/Z (M[³⁵Cl][⁸¹Br]+H)⁺: 289.0

### Compound 97: 3-(3-bromo-6-chloropyridin-2-yl)tetrahydrofuran-3-carboxamide

Compound 97 was prepared according to general procedure (IIa) starting from Compound 96 (2.00 g). The crude was purified by flash chromatography (Merck 60®, CyHex 100% to CyHex/EtOAc 0:100) to obtain Compound 97 (1.53 g 72%) as a white solid.

M/Z (M[³⁵Cl⁸¹Br]+H)⁺: 307.0

### Compound 98: 5′-chloro-4,5-dihydro-2H-spiro[furan-3,3′-pyrrolo[3,2-b]pyridin]-2′(1′H)-one

Compound 98 was prepared according to general procedure (III) starting from Compound 97 (1.53 g). The crude was purified by flash chromatography (Merck 60®, CyHex 100% to CyHex/EtOAc 0:100) to obtain Compound 98 (0.78 g, 69%) as a beige solid.

M/Z (M[³⁵Cl]+H)⁺: 225.1

### Compound 99: 5′-chloro-1′,2′,4,5-tetrahydro-2H-spiro[furan-3,3′-pyrrolo[3,2-b]pyridine]

Compound 99 was prepared according to general procedure (IX) starting from Compound 98 (780 mg). The crude was purified by flash chromatography (Merck 60®, CyHex 100% to EtOAc 100%) to obtain Compound 99 (725 mg, 99%) as a brown solid.

M/Z (M[³⁵Cl]+H)⁺: 211.0

### Compound 100: 1′,2′,4,5-tetrahydro-2H-spiro[furan-3,3′-pyrrolo[3,2-b]pyridine]-5′-carbonitrile

Compound 100 was prepared according to general procedure (IV) starting from Compound 99 (725 mg). The crude was purified by flash chromatography (Merck 60®, CyHex 100% to CyHex/EtOAc 0:100) to obtain Compound 100 (496 mg, 72%) as a yellow solid

M/Z (M+H)⁺: 202.1

### Compound 101: 1′-(4-chloro-3-fluorophenyl)-1′,2′,4,5-tetrahydro-2H-spiro[furan-3,3′-pyrrolo[3,2-b]pyridine]-5′-carbonitrile

Compound 101 was prepared according to general procedure (X) starting from Compound 100 (490 mg) and 4-bromo-1-chloro-2-fluorobenzene (1.0 eq.). The crude was purified by flash chromatography (Merck 60®, CyHex 100% to CyHex/EtOAc 60:40) to obtain Compound 101 (476 mg) as a white solid.

M/Z (M[³⁵Cl]+H)⁺: 330.2

### Compound 102: 1′-(4-chloro-3-fluorophenyl)-1′,2′,4,5-tetrahydro-2H-spiro[furan-3,3′-pyrrolo[3,2-b]pyridine]-5′-carboxamide

Compound 102 (388 mg, 75%) was obtained as a white solid from Compound 101 (490 mg) according to general procedure (IIb).

M/Z (M[³⁵Cl]+H)⁺: 348.2

### Compound 103: methyl 1′-(4-chloro-3-fluorophenyl)-1′,2′,4,5-tetrahydro-2H-spiro[furan-3,3′-pyrrolo[3,2-b]pyridine]-5′-carboxylate

Compound 103 was prepared according to general procedure (VI) starting from Compound 102 (680 mg). The crude was purified by flash chromatography (Merck 60®, CyHex 100% to CyHex/EtOAc 20:80) to obtain Compound 103 (436 mg, 62%) as a white solid.

M/Z (M[³⁵Cl]+H)⁺: 363.2

### Compound 104: 1′-(4-chloro-3-fluorophenyl)-1′,2′,4,5-tetrahydro-2H-spiro[furan-3,3′-pyrrolo[3,2-b]pyridine]-5′-carboxylic acid

Compound 104 was obtained (380 mg, 92%) as a white solid according to general procedure (VII) starting from Compound 103 (430 mg).

M/Z (M[³⁵Cl]+H)⁺: 349.2

### Example 58: 4-(1′-(4-chloro-3-fluorophenyl)-1′,2′,4,5-tetrahydro-2H-spiro[furan-3,3′-pyrrolo[3,2-b]pyridine]-5′-carbonyl)-3,3-dimethylpiperazin-2-one

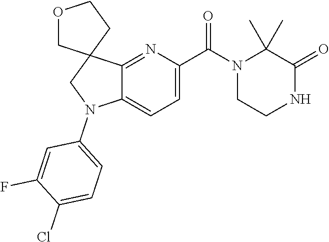 [SMILES: CC1(C)C(=O)NCCN1C(=O)c1ccc2c(n1)C1(CCOC1)CN2c1ccc(Cl)c(F)c1]

Example 58 was prepared according to general procedure (VIIIa) starting from Compound 104 (80 mg), 3,3-dimethylpiperazin-2-one (2.0 eq.) and N,N-diisopropylethylamine (3.0 eq.) in DCM. The crude was purified by flash chromatography (Merck 60®, CyHex 100% to CyHex/EtOAc 0:100 to EtOAc/MeOH 90:10) then further purified by preparative HPLC (Column B, H₂O+0.1% HCOOH/MeCN+0.1% HCOOH 80:20 to 0:100) and freeze dried to obtain Example 58 (42 mg, 40%) as a white solid.

¹H-NMR (CDCl₃, 300 MHz) δ: 1.87 (d, J 7.2 Hz, 6H); 2.11-2.19 (m, 1H, CH); 2.51-2.60 (m, 1H, CH); 3.54-3.84 (m, 4H, 2CH₂); 3.90-4.19 (m, 6H, 3CH₂); 6.17 (bs, 1H, NH); 6.95-7.05 (m, 2H, 2Ar); 7.37-7.44 (m, 2H, 2Ar); 7.63 (d, J 8.4 Hz, 1H, Ar).

M/Z (M[³⁵Cl]+H)⁺: 459.2.

Mp: 202-205° C.

### Example 59: methyl 6-(4-(1′-(4-chloro-3-fluorophenyl)-1′,2′,4,5-tetrahydro-2H-spiro[furan-3,3′-pyrrolo[3,2-b]pyridine]-5′-carbonyl)-3,3-dimethylpiperazin-1-yl)-2,4-dimethylnicotinate

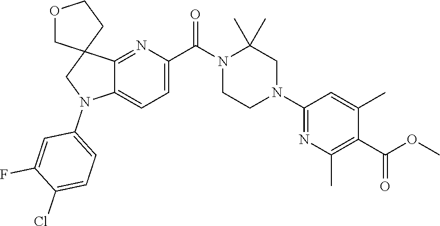 [SMILES: COC(=O)c1c(C)cc(N2CCN(C(=O)c3ccc4c(n3)C3(CCOC3)CN4c3ccc(Cl)c(F)c3)C(C)(C)C2)nc1C]

Example 59 was prepared according to general procedure (VIIIa) starting from Compound 104 (40 mg), Compound 10 (38 mg, 1.2 eq) and N,N-diisopropylethylamine (3.0 eq.) in DCM. The crude was purified by flash chromatography (Merck 60®, CyHex 100% to CyHex/EtOAc 0:100) to obtain Example 59 (65 mg) as a white solid.

¹H-NMR (CDCl₃, 300 MHz) δ: 1.63 (s, 6H, 2CH₃); 2.11-2.22 (m, 1H, OCH₂—CHₐH_{b}); 2.35 (s, 3H, CH₃); 2.50 (s, 3H, CH₃); 2.59-2.64 (m, 1H, OCH₂—CHₐH_{b}); 3.64-3.67 (m, 2H, OCH₂); 3.86-4.20 (m, 13H, 4 NCH₂, O—CH₂, COOCH₃); 6.14 (s, 1H, Ar); 6.94-7.04 (m, 2H, 2Ar); 7.36-7.43 (m, 2H, 2Ar); 7.55 (d, J 8.4 Hz, 1H, Ar).

M/Z (M[³⁵Cl]+H)⁺: 608.5.

### Example 60: 6-(4-(1′-(4-chloro-3-fluorophenyl)-1′,2′,4,5-tetrahydro-2H-spiro[furan-3,3′-pyrrolo[3,2-b]pyridine]-5′-carbonyl)-3,3-dimethylpiperazin-1-yl)-2,4-dimethylnicotinic acid hydrochloride

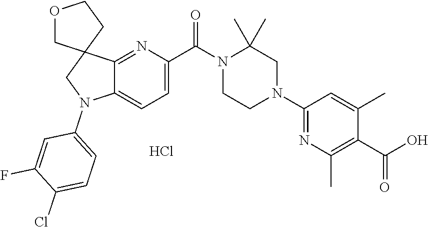 [SMILES: Cc1cc(N2CCN(C(=O)c3ccc4c(n3)C3(CCOC3)CN4c3ccc(Cl)c(F)c3)C(C)(C)C2)nc(C)c1C(=O)O ; Cl]

Example 60 was prepared according to general procedure (XI) starting from Example 59 (80 mg). The crude was purified by flash chromatography (Merck 60®, DCM 100% to DCM/MeOH 80:20) then further purified by preparative HPLC (Column B, H₂O+0.1% HCOOH/MeCN+0.1% HCOOH 80:20 to 0:100) and freeze dried with aqueous HCl to obtain Example 60 (15 mg, 22% over 3 steps) as a yellow solid.

¹H-NMR (DMSO-d₆, 300 MHz) δ: 1.49 (s, 6H, 2CH₃); 2.15-2.22 (m, 1H, CH); 2.26 (s, 3H, CH₃); 2.31-2.36 (m, 1H, CH); 2.40 (s, 3H, CH₃); 3.51-3.54 (m, 2H, CH₂); 3.81-4.10 (m, 12H, 6CH₂); 6.44 (bs, 1H, Ar); 7.20 (dd, J 8.7, 2.1 Hz, 1H, Ar); 7.33-7.40 (m, 2H, 2Ar); 7.56-7.60 (m, 2H, 2Ar).

M/Z (M[³⁵Cl]+H)⁺: 594.4.

### Compound 105: cis-5-chloro-3a-methyl-3,3a,8,8a-tetrahydro-2H-furo[3′,2′:4,5]pyrrolo[3,2-b]pyridine

In a screw cap vial, to a solution of 3-methyltetrahydrofuran-2-ol (1.10 g) in 35 mL H₂SO₄ (4% aqueous) was added 2-chloro-5-hydrazineylpyridine hydrochloride (1.5 eq). The reaction mixture was heated at 120° C. for 1 hour, then neutralized in 200 mL NaHCO₃ (sat. aq.). The mixture was extracted with EtOAc (4*50 mL), dried over MgSO₄ and concentrated to dryness. The crude was purified by column chromatography (Merck 60®, CyHex 100% to EtOAc 100%) to obtain Compound 105 (1.31 g, 54%) as a brown solid.

M/Z (M[³⁵Cl]+H)⁺: 211.1.

### Compound 106: cis-3a-methyl-3,3a,8,8a-tetrahydro-2H-furo[3′,2′:4,5]pyrrolo[3,2-b]pyridine-5-carbonitrile

Compound 106 was prepared according to general procedure (IV) starting from Compound 105 (1.01 g). The crude was purified by flash chromatography (Merck 60®, CyHex 100% to CyHex/EtOAc 30:70) to obtain Compound 106 (886 mg, 92%) as a brown solid

M/Z (M+H)⁺: 202.0

### Compound 107: cis-8-(4-chloro-3-fluorophenyl)-3a-methyl-3,3a,8,8a-tetrahydro-2H-furo[3′,2′:4,5]pyrrolo[3,2-b]pyridine-5-carboxamide

Compound 107 was prepared according to general procedure (X) starting from Compound 106 (300 mg) and 4-bromo-1-chloro-2-fluorobenzene (1.1 eq.). The crude was purified by flash chromatography (Merck 60®, CyHex 100% to EtOAc 100%) to obtain Compound 107 (304 mg, 59%) as a yellow solid.

M/Z (M[³⁵Cl]+H)⁺: 348.1

### Compound 108: methyl cis-8-(4-chloro-3-fluorophenyl)-3a-methyl-3,3a,8,8a-tetrahydro-2H-furo[3′,2′:4,5]pyrrolo[3,2-b]pyridine-5-carboxylate

Compound 108 was prepared according to general procedure (VI) starting from Compound 107 (304 mg). The crude was purified by flash chromatography (Merck 60®, CyHex 100% to CyHex/EtOAc 25:75) to obtain Compound 108 (292 mg, 92%) as a white solid.

M/Z (M[³⁵Cl]+H)⁺: 363.2

### Compound 109: cis-8-(4-chloro-3-fluorophenyl)-3a-methyl-3,3a,8,8a-tetrahydro-2H-furo[3′,2′:4,5]pyrrolo[3,2-b]pyridine-5-carboxylic acid

Compound 109 was obtained (264 mg, 94%) as a white solid according to general procedure (VII) starting from Compound 108 (292 mg).

M/Z (M[³⁵Cl]+H)⁺: 349.0

### Example 61: cis-4-(8-(4-chloro-3-fluorophenyl)-3a-methyl-3,3a,8,8a-tetrahydro-2H-furo[3′,2′:4,5]pyrrolo[3,2-b]pyridine-5-carbonyl)-3,3-dimethylpiperazin-2-one

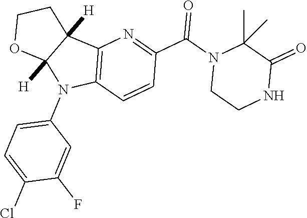 [SMILES: CC1(C)C(=O)NCCN1C(=O)c1ccc2c(n1)[C@H]1CCO[C@H]1N2c1ccc(Cl)c(F)c1]

Example 61 was prepared according to general procedure (VIIIa) starting from Compound 109 (258 mg), 3,3-dimethylpiperazin-2-one (1.3 eq.) and N,N-diisopropylethylamine (3.0 eq.) in DCM/THF (1:1). The crude was purified by flash chromatography (Merck 60®, CyHex 100% to EtOAc 100%) then further purified by preparative HPLC (Column B, H₂O+0.1% HCOOH/MeCN+0.1% HCOOH 80:20 to 0:100) and freeze dried with MeOH/water to obtain Example 61 (210 mg, 62%) as a white solid.

¹H-NMR (CDCl₃, 300 MHz) δ: 1.48 (s, 3H, CH₃); 1.67 (s, 6H, 2CH₃); 2.04-2.15 (m, 1H, CH₂—CH₂-0); 2.29-2.30 (m, 1H, CH₂—CH₂-0); 3.37-3.52 (m, 3H, CH₂—CH₂—O, CH₂); 3.96-4.03 (m, 1H, CH₂—CH₂-0); 5.72 (s, 1H, CH); 7.32 (dd, 1H, J 8.7, J 2.1 Hz, 1H, Ar); 7.42-7.51 (m, 3H, 3Ar); 7.61 (t, J 8.7 Hz, 1H, Ar); 8.04-8.06 (m, 1H, NHCO). One CH₂ signal not observed.

M/Z (M[³⁵Cl]+H)⁺:459.2

MP: 243-245° C.

### Example 62 and Example 63: 4-((3aR,8aR)-8-(4-chloro-3-fluorophenyl)-3a-methyl-3,3a,8,8a-tetrahydro-2H-furo[3′,2′:4,5]pyrrolo[3,2-b]pyridine-5-carbonyl)-3,3-dimethylpiperazin-2-one and 4-((3aS,8aS)-8-(4-chloro-3-fluorophenyl)-3a-methyl-3,3a,8,8a-tetrahydro-2H-furo[3′,2′:4,5]pyrrolo[3,2-b]pyridine-5-carbonyl)-3,3-dimethylpiperazin-2-one

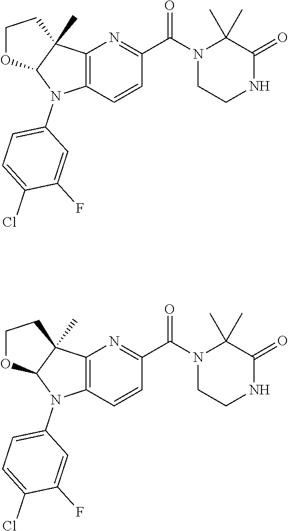 [SMILES: CC1(C)C(=O)NCCN1C(=O)c1ccc2c(n1)[C@@]1(C)CCO[C@H]1N2c1ccc(Cl)c(F)c1 ; CC1(C)C(=O)NCCN1C(=O)c1ccc2c(n1)[C@]1(C)CCO[C@@H]1N2c1ccc(Cl)c(F)c1]

Example 61 was purified by SFC, using ChiralPak IG 5 μm (250*20 mm) as the stationary phase and CO₂/Ethanol (70:30, 120 mL/min) as the mobile phase, to obtain:

Example 62 (105 mg, 50%, ee>99.5%), first eluting enantiomer (T_{R}=6.25 min)

¹H-NMR (DMSO-d₆, 300 MHz) δ: 1.47 (s, 3H, CH₃); 1.66 (s, 6H, 2CH₃); 2.04-2.14 (m, 1H, CH₂—CH₂-0); 2.25-2.31 (m, 1H, CH₂—CH₂-0); 3.38-3.44 (m, 3H, CH₂—CH₂-0, CH₂); 3.48-3.50 (m, 2H, CH₂); 3.96-4.01 (m, 1H, CH₂—CH₂-0); 5.70 (s, 1H, CH); 7.31 (dd, J 9.0, J 2.1 Hz, 1H, Ar); 7.42-7.45 (m, 2H, 2Ar); 7.48 (d, J 8.4 Hz, 1H, Ar); 7.58 (t, J 8.7 Hz, 1H, Ar); 8.03-8.06 (m, 1H, NHCO).

M/Z (M[³⁵Cl]+H)⁺:459.3

MP: 245° C.

Example 63 (105 mg, 50%, ee>99.5%), second eluting enantiomer (T_{R}=7.08 min)

¹H-NMR (DMSO-d₆, 300 MHz) δ: 1.48 (s, 3H, CH₃); 1.67 (s, 6H, 2CH₃); 2.04-2.15 (m, 1H, CH₂—CH₂-0); 2.26-2.31 (m, 1H, CH₂—CH₂-0); 3.36-3.43 (m, 3H, CH₂—CH₂-0, CH₂); 3.49-3.52 (m, 2H, CH₂); 3.97-4.02 (m, 1H, CH₂—CH₂-0); 5.71 (s, 1H, CH); 7.34-7.30 (m, 1H, Ar); 7.42-7.41 (m, 2H, 2Ar); 7.49 (d, J 8.4 Hz, 1H, Ar); 7.59 (t, J 9.0 Hz, 1H, Ar); 8.03-8.06 (m, 1H, NHCO).

M/Z (M[³⁵Cl]+H)⁺:459.3

MP: 245° C.

### Compound 110: tert-butyl 4-(5-(2-methoxy-2-oxoethyl)pyridin-2-yl)-2,2-dimethylpiperazine-1-carboxylate

Compound 110 was prepared according to general procedure (Xb), starting from methyl 2-(6-chloropyridin-3-yl)acetate (200 mg) and tert-butyl 2,2-dimethylpiperazine-1-carboxylate (1.2 eq.). The residue was purified by flash chromatography (Interchim®50 μm, CyHex 100% to CyHex/EtOAc 50:50) to obtain Compound 110 (236 mg, 60%) as a clear oil.

M/Z (M+H)⁺:364.3

### Compound 111: methyl 2-(6-(3,3-dimethylpiperazin-1-yl)pyridin-3-yl)acetate hydrochloride

Compound 111 was obtained as a white solid (188 mg, 98%) according to general procedure (XII) starting from Compound 110 (233 mg) in DCM using HCl in Et₂O.

M/Z (M+H)⁺:264.1

### Example 64: methyl 2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)acetate

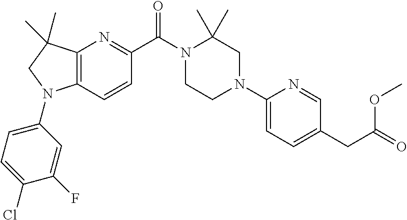 [SMILES: COC(=O)Cc1ccc(N2CCN(C(=O)c3ccc4c(n3)C(C)(C)CN4c3ccc(Cl)c(F)c3)C(C)(C)C2)nc1]

Example 64 was prepared according to general procedure (VIIIa) starting from Compound 68 (55 mg), Compound 111 (1.2 eq.), and N,N-diisopropylethylamine (4.0 eq.) in DCM. The crude was purified by flash chromatography (Merck 60®, CyHex 100% to CyHex/EtOAc 50:50) to obtain Example 64 (66 mg, 68%) as a yellow oil.

¹H-NMR (DMSO-d₆, 300 MHz) δ: 1.36 (s, 6H, 2CH₃); 1.50 (s, 6H, 2CH₃); 3.48-3.54 (m, 4H, 2N—CH₂); 3.61 (s, 3H, OCH₃); 3.83-3.86 (m, 6H, 2N—CH₂+CH₂C(O)); 6.60 (d, 1H, J 2.7 Hz, Ar); 7.17-7.21 (m, 1H, Ar); 7.30-7.37 (m; 2H, 2 Ar); 7.43-7.47 (m, 1H, Ar); 7.52-7.58 (m, 2H, 2Ar); 7.96-7.97 (m, 1H, Ar).

M/Z (M[³⁵Cl]+H)⁺:566.5

### Example 65: 2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)acetic acid

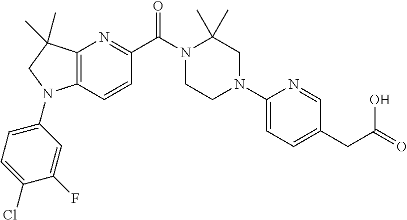 [SMILES: CC1(C)CN(c2ccc(Cl)c(F)c2)c2ccc(C(=O)N3CCN(c4ccc(CC(=O)O)cn4)CC3(C)C)nc21]

Example 65 was prepared according to general procedure (VII) starting from Example 64 (66 mg). The crude was purified by preparative HPLC (Column B, H₂O+0.1% HCOOH/MeCN+0.1% HCOOH 95:5 to 0:100) and freeze dried with water to obtain Example 65 (37 mg, 58%) as a white solid.

¹H-NMR (DMSO-d₆, 300 MHz) δ: 1.36 (s, 6H, 2CH₃); 1.50 (s, 6H, 2CH₃); 3.42 (s, 2H, CH₂C(O)); 3.49-3.53 (m, 2H, N—CH₂); 3.81-3.86 (m, 6H, 3N—CH₂); 6.59 (d, J 8.7 Hz, 1H Ar); 7.17-7.21 (m, 1H, Ar); 7.30-7.37 (m, 2H, 2Ar); 7.44 (dd, J 8.7, 2.3 Hz, 1H, Ar); 7.52-7.58 (m, 2H, 2Ar); 7.95 (d, J 2.3 Hz, 1H, Ar). COOH was not observed.

M/Z (M[³⁵Cl]+H)⁺: 552.5.

### Example 66: ethyl 5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-1,3,4-thiadiazole-2-carboxylate

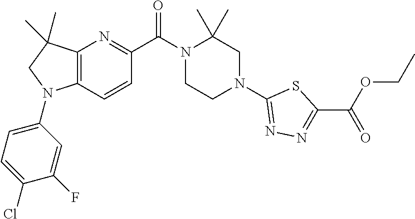 [SMILES: CCOC(=O)c1nnc(N2CCN(C(=O)c3ccc4c(n3)C(C)(C)CN4c3ccc(Cl)c(F)c3)C(C)(C)C2)s1]

A suspension of Example 22 (75 mg), Ethyl 5-chloro-1,3,4-thiadiazole-2-carboxylate (1.5 eq.) and Potassium Carbonate (3 eq.) in DMF (0.1 M) was heated at 80° C. for 16 hours. The reaction mixture was quenched in NH₄Cl (sat. aq., 50 mL), extracted with EtOAc (50 mL). The organic layer was washed with brine (50 mL), dried over MgSO₄ and concentrated under reduced pressure. The residue was purified by flash chromatography (Interchim® 50 μm, CyHex 100% to CyHex/EtOAc 40:60) to obtain Example 66 (70 mg, 74%) as a clear oil.

¹H-NMR (DMSO-d₆, 300 MHz) δ: 1.29-1.35 (m, 9H, 2CH₃+CH₂—CH₃); 1.54 (s, 6H, 2CH₃); 3.67-3.70 (m, 2H, N—CH₂); 3.84-3.89 (m, 6H, 3N—CH₂); 4.35 (q, 2H, J 4.2 Hz, CH₂—CH₃); 7.17-7.21 (m, 1H, Ar); 7.30-7.35 (m, 1H, Ar); 7.37-7.40 (m, 1H, Ar); 7.52-7.58 (m, 2H, 2Ar).

M/Z (M[³⁵Cl]+H)⁺: 573.4

### Example 67: (1-(4-chloro-3-fluorophenyl)-3,3-dimethyl-2,3-dihydro-1H-pyrrolo[3,2-b]pyridin-5-yl)(2,2-dimethyl-4-(1,3,4-thiadiazol-2-yl)piperazin-1-yl)methanone

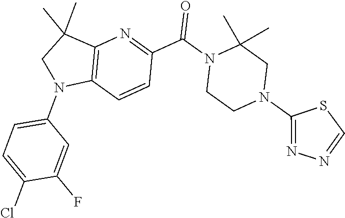 [SMILES: CC1(C)CN(c2ccc(Cl)c(F)c2)c2ccc(C(=O)N3CCN(c4nncs4)CC3(C)C)nc21]

Example 67 was prepared according to general procedure (VII) starting from Example 66 (67 mg). The crude was purified by preparative HPLC (Column B, H₂O+0.1% HCOOH/MeCN+0.1% HCOOH 80:20 to 0:100) and freeze dried with water to obtain Example 67 (36 mg, 62%) as a white solid.

¹H-NMR (DMSO-d₆, 300 MHz) δ: 1.35 (s, 6H, 2*CH₃); 1.53 (s, 6H, 2*CH₃); 3.58-3.66 (m, 2H, N—CH₂); 3.75 (s, 2H, N—CH₂); 3.78-3.86 (m, 2H, N—CH₂); 3.85 (s, 2H, N—CH₂); 7.19 (dd, J 8.9, 1.9 Hz, 1H, Ar); 7.32 (dd, J 12.0, 2.4 Hz, 1H, Ar); 7.38 (d, J 8.4 Hz, 1H, Ar); 7.50-7.60 (m, 2H, 2Ar); 8.75 (s, 1H, Ar).

M/Z (M[³⁵Cl]+H)⁺: 501.3.

### Compound 112: ethyl 2-(4-(tert-butoxycarbonyl)-3,3-dimethylpiperazin-1-yl)-4-methylthiazole-5-carboxylate

Compound 112 was prepared according to general procedure (Xb), starting from ethyl 2-bromo-4-methylthiazole-5-carboxylate (200 mg) and tert-butyl 2,2-dimethylpiperazine-1-carboxylate (1.2 eq.). The residue was purified by flash chromatography (Interchim®50 μm, CyHex 100% to CyHex/EtOAc 80:20) to obtain Compound 112 (307 mg, 80%) as an orange oil.

M/Z (M+H)⁺:384.3

### Compound 113: ethyl 2-(3,3-dimethylpiperazin-1-yl)-4-methylthiazole-5-carboxylate

Compound 113 was obtained as a yellow oil (300 mg) according to general procedure (XII) starting from Compound 112 (233 mg) in DCM using TFA.

M/Z (M+H)⁺:284.2

### Example 68: ethyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylthiazole-5-carboxylate

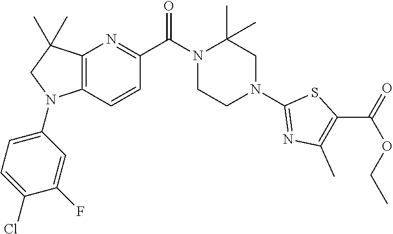 [SMILES: CCOC(=O)c1sc(N2CCN(C(=O)c3ccc4c(n3)C(C)(C)CN4c3ccc(Cl)c(F)c3)C(C)(C)C2)nc1C]

Example 68 was prepared according to general procedure (VIIIa) starting from Compound 68 (55 mg), Compound 113 (1.1 eq.), and N,N-diisopropylethylamine (3.0 eq.) in DCM. The crude was purified by flash chromatography (Merck 60®, CyHex 100% to CyHex/EtOAc 50:50) to obtain Example 68 (42 mg, 42%) as a yellow oil.

¹H-NMR (DMSO-d₆, 300 MHz) δ: 1.24 (t, 3H, J 7.2 Hz, CH₂CH₃); 1.35 (s, 6H, 2CH₃); 1.52 (s, 6H, 2CH₃); 3.54-3.59 (m, 2H, N—CH₂); 3.76-3.86 (m, 6H, 3N—CH₂); 4.18 (q, 2H, J 7.2 Hz, CH₂CH₃); 7.17-7.21 (m, 1H, Ar); 7.30-7.35 (m; 2H, 2Ar); 7.35-7.40 (m, 1H, Ar); 7.52-7.58 (m, 2H, 2Ar). One CH₃ was not observed

M/Z (M[³⁵Cl]+H)⁺:586.4

### Example 69: 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylthiazole-5-carboxylic acid

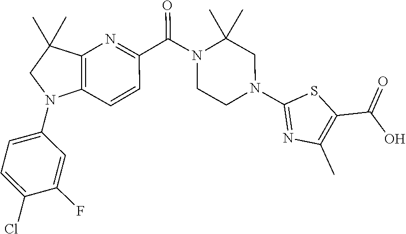 [SMILES: Cc1nc(N2CCN(C(=O)c3ccc4c(n3)C(C)(C)CN4c3ccc(Cl)c(F)c3)C(C)(C)C2)sc1C(=O)O]

Example 69 was prepared according to general procedure (VII) starting from Example 68 (42 mg). The crude was purified by preparative HPLC (Column B, H₂O+0.1% HCOOH/MeCN+0.1% HCOOH 80:20 to 0:100) and freeze dried with water to obtain Example 69 (10 mg, 25%) as a yellow solid.

¹H-NMR (DMSO-d₆, 300 MHz) δ: 1.35 (s, 6H, 2CH₃); 1.52 (s, 6H, 2CH₃); 2.44 (s, 3H, CH₃); 3.53-3.57 (m, 2H, NCH₂); 3.76 (s, 2H, NCH₂); 3.81-3.86 (m, 4H, 2 NCH₂); 7.17-7.21 (m, 1H, Ar); 7.33 (dd, J 12.1, 2.5 Hz, 1H, Ar); 7.38 (d, J 8.4 Hz, 1H, Ar); 7.52-7.58 (m, 2H, 2Ar). COOH was not observed.

M/Z (M[³⁵Cl]+H)⁺: 558.4.

### Compound 114: ethyl 2-(2-bromo-4-methylthiazol-5-yl)acetate

To a solution of ethyl 2-(2-amino-4-methylthiazol-5-yl)acetate (225 mg) and Copper^{(II)} bromide (1.2 eq.) in MeCN (0.1 M) was added tert-butyl nitrite (1.5 eq.). The reaction mixture was sealed and heated at 60° C. for 2 hours. The reaction mixture was concentrated under reduced pressure and the residue was purified by flash chromatography (Interchim®50 μm, CyHex 100% to CyHex/EtOAc 70:30) to obtain Compound 114 (191 mg, 64%) as an orange oil.

M/Z ([⁸¹Br]M+H)⁺:266.0

### Compound 115: tert-butyl 4-(5-(2-ethoxy-2-oxoethyl)-4-methylthiazol-2-yl)-2,2-dimethylpiperazine-1-carboxylate

Compound 115 was prepared according to general procedure (Xb), starting from Compound 114 (191 mg) and tert-butyl 2,2-dimethylpiperazine-1-carboxylate (1.2 eq.). The residue was purified by flash chromatography (Interchim® 50 μm, CyHex 100% to CyHex/EtOAc 70:30) to obtain Compound 115 (106 mg, 37%) as a yellow oil.

M/Z (M+H)⁺: 398.3

### Compound 116: ethyl 2-(2-(3,3-dimethylpiperazin-1-yl)-4-methylthiazol-5-yl)acetate

Compound 116 was obtained as an orange oil (70 mg) according to general procedure (XII) starting from Compound 115 (106 mg) in DCM using TFA.

M/Z (M+H)⁺:298.2

### Example 70: ethyl 2-(2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylthiazol-5-yl)acetate

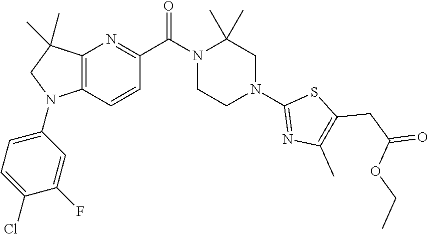 [SMILES: CCOC(=O)Cc1sc(N2CCN(C(=O)c3ccc4c(n3)C(C)(C)CN4c3ccc(Cl)c(F)c3)C(C)(C)C2)nc1C]

Example 70 was prepared according to general procedure (VIIIa) starting from Compound 68 (55 mg), Compound 115 (1.4 eq.), and N,N-diisopropylethylamine (4.0 eq.) in DCM. The crude was purified by flash chromatography (Merck 60®, CyHex 100% to CyHex/EtOAc 40:60) to obtain Example 70 (78 mg, 76%) as a yellow oil.

¹H-NMR (DMSO-d₆, 300 MHz) δ: 1.18 (t, 3H, J 7.2 Hz, CH₂CH₃), 1.34 (s, 6H, 2CH₃); 1.50 (s, 6H, 2CH₃); 2.05 (s, 3H, CH₃); 3.49-3.64 (m, 6H, 3N—CH₂); 3.77-3.81 (m, 2H, N—CH₂); 3.84 (s, 2H, CH₂C(O)); 4.07 (q, 2H, J 7.2 Hz, CH₂CH₃); 7.16-7.20 (m, 1H, Ar); 7.27-7.32 (m, 1H, Ar); 7.35-7.37 (m, 1H, Ar); 7.51-7.57 (m, 2H, 2Ar).

M/Z (M[³⁵Cl]+H)⁺:600.4

### Example 71: 2-(2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylthiazol-5-yl)acetic acid

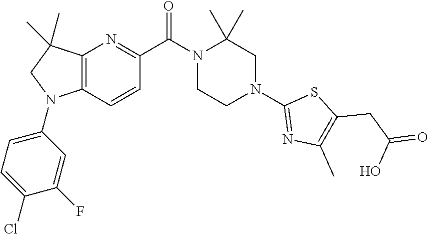 [SMILES: Cc1nc(N2CCN(C(=O)c3ccc4c(n3)C(C)(C)CN4c3ccc(Cl)c(F)c3)C(C)(C)C2)sc1CC(=O)O]

Example 71 was prepared according to general procedure (VII) starting from Example 70 (78 mg). The crude was purified by preparative HPLC (Column B, H₂O+0.1% HCOOH/MeCN+0.1% HCOOH 80:20 to 0:100) and freeze dried with water to obtain Example 71 (30 mg, 40%) as a beige solid.

¹H-NMR (DMSO-d₆, 300 MHz) δ: 1.35 (s, 6H, 2CH₃); 1.51 (s, 6H, 2CH₃); 2.06 (s, 3H, CH₃); 3.46-3.50 (m, 2H, CH₂); 3.56 (s, 2H, CH₂); 3.63 (s, 2H, CH₂); 3.77-3.81 (m, 2H, CH₂); 3.86 (s, 2H, CH₂); 7.16-7.21 (m, 1H, Ar); 7.32 (dd, J 12.5, 2.6 Hz, 1H, Ar); 7.37 (d, J 8.4 Hz, 1H, Ar); 7.52-7.58 (m, 2H, 2Ar). COOH was not observed.

M/Z (M[³⁵Cl]+H)⁺: 572.4.

### Example 72: methyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-5-methylthiazole-4-carboxylate

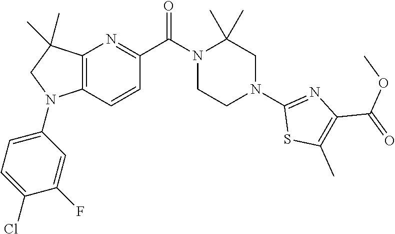 [SMILES: COC(=O)c1nc(N2CCN(C(=O)c3ccc4c(n3)C(C)(C)CN4c3ccc(Cl)c(F)c3)C(C)(C)C2)sc1C]

Example 72 was prepared according to general procedure (Xb), starting from Example 22 (75 mg) methyl 2-bromo-5-methylthiazole-4-carboxylate (1.5 eq.). The residue was purified by flash chromatography (Interchim®50 μm, CyHex 100% to CyHex/EtOAc 50:50) to obtain Example 72 (57 mg) as a brown solid.

¹H-NMR (CDCl₃, 300 MHz) δ: 1.41 (s, 6H, (CH₃)₂); 1.65 (s, 6H, (CH₃)₂); 2.63 (s, 3H, CH₃); 3.64-3.70 (m, 4H, 2N—CH₂); 3.77 (s, 2H, N—CH₂); 3.88 (s, 3H, OCH₃); 3.89-3.96 (m, 2H, CH₂); 6.92-6.98 (m, 2H, 2Ar); 7.32-7.38 (m, 2H, 2 Ar); 7.50-7.52 (m, 1H, Ar).

M/Z (M[³⁵Cl]+H)⁺: 572.4.

### Example 73: 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-5-methylthiazole-4-carboxylic acid hydrochloride

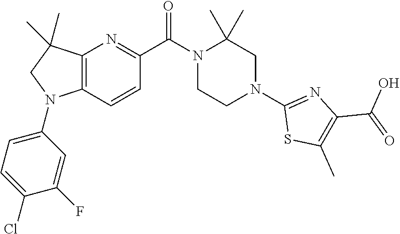 [SMILES: Cc1sc(N2CCN(C(=O)c3ccc4c(n3)C(C)(C)CN4c3ccc(Cl)c(F)c3)C(C)(C)C2)nc1C(=O)O]

Example 73 was prepared according to general procedure (VII) starting from Example 72 (57 mg). The crude was purified by preparative HPLC (Column B, H₂O+0.1% HCOOH/MeCN+0.1% HCOOH 80:20 to 0:100) and freeze dried with HCl (aq. 0.1N) to obtain Example 73 (30 mg, 30%) as a yellow solid.

¹H-NMR (DMSO-d₆, 300 MHz) δ: 1.34 (s, 6H, (CH₃)₂); 1.51 (s, 6H, (CH₃)₂); 2.53 (s, 3H, CH₃); 3.49-3.54 (m, 2H, CH₂); 3.63-3.68 (m, 2H, CH₂); 3.77-3.88 (m, 4H, 2*CH₂); 7.18 (dd, J 9.0, 2.1 Hz, 1H, Ar); 7.30-7.38 (m, 2H, 2Ar); 7.52-7.59 (m, 2H, 2Ar). COOH and Hydrochloric salt are not observed.

M/Z (M[³⁵Cl]+H)⁺: 558.5.

### Example 74: 1-(1-(4-chloro-3-fluorophenyl)-3,3-dimethyl-2,3-dihydro-1H-pyrrolo[3,2-b]pyridine-5-carbonyl)-7,7-dimethyl-1,4-diazepan-5-one

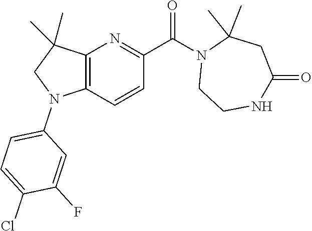 [SMILES: CC1(C)CN(c2ccc(Cl)c(F)c2)c2ccc(C(=O)N3CCNC(=O)CC3(C)C)nc21]

Example 74 was prepared according to general procedure (VIIIa) starting from Compound 68 (80 mg), 7,7-dimethyl-1,4-diazepan-5-one (1.0 eq.), and N,N-diisopropylethylamine (3.0 eq.) in DMF. The crude was purified by flash chromatography (Merck 60®, DCM 100% to DCM/MeOH 90:10), then precipitated from DMSO (5 mL) with water (30 mL) and washed with water to obtain Example 74 (45 mg, 41%) as a beige solid.

¹H-NMR (DMSO-d₆, 300 MHz) δ: 1.33 (s, 6H, (CH₃)₂); 1.59 (s, 6H, (CH₃)₂); 2.60-2.65 (m, 2H, CH₂); 3.40-3.47 (m, 2H, CH₂); 3.66-3.73 (m, 2H, CH₂); 3.84 (s, 2H, CH₂); 7.16-7.19 (m, 1H, Ar); 7.29-7.40 (m, 3H, NH+Ar); 7.52-7.57 (m, 2H, 2Ar).

M/Z (M[³⁵Cl]+H)⁺: 445.9

### Example 75: 8-(1-(4-chloro-3-fluorophenyl)-3,3-dimethyl-2,3-dihydro-1H-pyrrolo[3,2-b]pyridine-5-carbonyl)-2,8-diazaspiro[4.5]decan-1-one

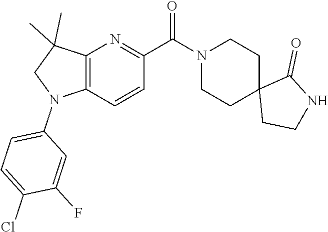 [SMILES: CC1(C)CN(c2ccc(Cl)c(F)c2)c2ccc(C(=O)N3CCC4(CCNC4=O)CC3)nc21]

Example 75 was prepared according to general procedure (VIIIa) starting from Compound 68 (80 mg), 2,8-diazaspiro[4.5]decan-1-one hydrochloride (1.1 eq.), and N,N-diisopropylethylamine (5.0 eq.) in DMF. The crude was purified by flash chromatography (Merck 60®, DCM 100% to DCM/MeOH 90:10), then precipitated from DMSO (5 mL) with water (30 mL) and washed with water to obtain Example 75 (70 mg, 61%) as a beige solid.

¹H-NMR (DMSO-d₆, 300 MHz) δ: 1.33-1.50 (m, 8H, CH₂+(CH₃)₂); 1.62-1.79 (m, 2H, CH₂); 1.97-2.12 (m, 2H, CH₂); 2.97-3.10 (m, 1H, CHₐH_{b}); 3.14-3.24 (m, 3H, CHₐH_{b}+CH₂); 3.86 (s, 2H, CH₂); 3.94-4.08 (m, 1H, CH); 4.26-4.37 (m, 1H, CH); 7.18 (dd, 1H, J 2.4, 8.7 Hz, Ar); 7.30-7.39 (m, 2H, 2Ar); 7.51-7.61 (m, 3H, NH+Ar).

M/Z (M[³⁵Cl]+H)⁺: 457.9.

### Example 76: 8-(1-(4-chloro-3-fluorophenyl)-3,3-dimethyl-2,3-dihydro-1H-pyrrolo[3,2-b]pyridine-5-carbonyl)-2,8-diazaspiro[4.5]decan-3-one

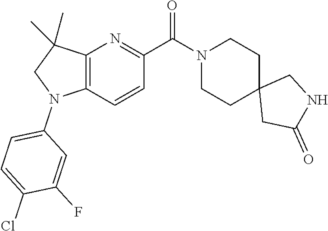 [SMILES: CC1(C)CN(c2ccc(Cl)c(F)c2)c2ccc(C(=O)N3CCC4(CC3)CNC(=O)C4)nc21]

Example 76 was prepared according to general procedure (VIIIa) starting from Compound 68 (80 mg), 2,8-diazaspiro[4.5]decan-3-one hydrochloride (1.1 eq.), and N,N-diisopropylethylamine (5.0 eq.) in DMF. The crude was purified by flash chromatography (Merck 60®, DCM 100% to DCM/MeOH 90:10), then precipitated from DMSO (5 mL) with water (30 mL) and washed with water to obtain Example 76 (59 mg, 48%) as a beige solid.

¹H-NMR (DMSO-d₆, 300 MHz) δ: 1.33 (s, 6H, (CH₃)₂); 1.54-1.63 (m, 4H, 2*CH₂); 2.12 (s, 2H, CH₂); 3.10 (s, 2H, CH₂); 3.43-3.77 (m, 4H, 2*N—CH₂); 3.85 (s, 2H, N—CH₂); 7.17 (dd, 1H, J 1.8, 8.7 Hz, Ar); 7.29-7.37 (m, 2H, 2Ar); 7.51-7.57 (m, 3H, Ar+NH).

M/Z (M[³⁵Cl]+H)⁺: 457.9.

### Example 77: 1-(1-(1-(4-chloro-3-fluorophenyl)-3,3-dimethyl-2,3-dihydro-1H-pyrrolo[3,2-b]pyridine-5-carbonyl)piperidin-4-yl)imidazolidin-2-one

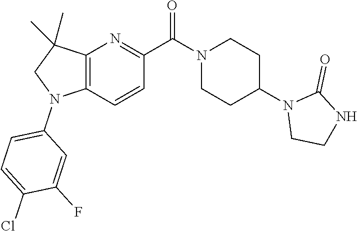 [SMILES: CC1(C)CN(c2ccc(Cl)c(F)c2)c2ccc(C(=O)N3CCC(N4CCNC4=O)CC3)nc21]

Example 77 was prepared according to general procedure (VIIIa) starting from Compound 68 (80 mg), 1-(piperidin-4-yl)imidazolidin-2-one (1.1 eq.), and N,N-diisopropylethylamine (3.0 eq.) in DMF. The crude was purified by flash chromatography (Merck 60®, DCM 100% to DCM/MeOH 90:10), then precipitated from DMSO (5 mL) with water (30 mL) and washed with water to obtain Example 77 (28 mg, 24%) as a beige solid.

¹H-NMR (DMSO-d₆, 300 MHz) δ: 1.34 (s, 6H, (CH₃)₂); 1.49-1.75 (m, 4H, 2*CH₂); 2.77-2.88 (m, 1H, CHₐH_{b}); 3.00-3.15 (m, 1H, CHₐH_{b}); 3.17-3.30 (m, 4H, 2*CH₂); 3.73-3.86 (m, 3H, CH₂, CHₐH_{b}); 4.07-4.18 (m, 1H, CHₐH_{b}); 4.47-4.62 (m, 1H, N—CH); 6.27 (s, 1H, NH), 7.15-7.21 (m, 1H, Ar); 7.30-7.40 (m, 2H, 2Ar); 7.51-7.58 (m, 2H, 2Ar).

M/Z (M[³⁵Cl]+H)⁺: 472.8.

### Example 78: ethyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-(trifluoromethyl)thiazole-5-carboxylate

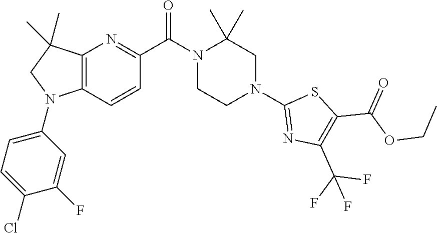 [SMILES: CCOC(=O)c1sc(N2CCN(C(=O)c3ccc4c(n3)C(C)(C)CN4c3ccc(Cl)c(F)c3)C(C)(C)C2)nc1C(F)(F)F]

Example 78 was prepared according to general procedure (Xb), starting from Example 22 (100 mg) ethyl 2-bromo-4-(trifluoromethyl)thiazole-5-carboxylate (1.5 eq.). The residue was purified by flash chromatography (Interchim®50 μm, CyHex 100% to EtOAc 100%) to obtain Example 78 (120 mg, 85%) as a clear oil.

¹H-NMR (CDCl₃, 300 MHz) δ: 1.35 (t, 3H, J 7.2 Hz, CH₂CH₃); 1.43 (s, 6H, (CH₃)₂); 1.65 (s, 6H, (CH₃)₂); 3.65-3.68 (m, 2H, N—CH₂); 3.73-3.83 (m, 4H, 2*N—CH₂); 4.00-4.03 (m, 2H, N—CH₂); 4.32 (q, 2H, J 7.2 Hz, CH₂CH₃); 6.92-7.01 (m, 2H, 2Ar); 7.34-7.40 (m, 2H, 2Ar); 7.54 (d, 1H, J 8.4 Hz, Ar).

M/Z (M[³⁵Cl]+H)⁺: 640.9.

### Example 79: 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-(trifluoromethyl)thiazole-5-carboxylic acid

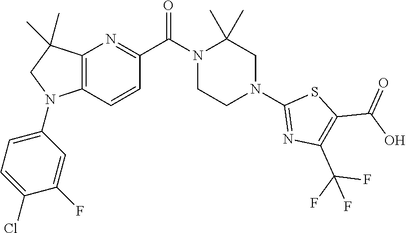 [SMILES: CC1(C)CN(c2ccc(Cl)c(F)c2)c2ccc(C(=O)N3CCN(c4nc(C(F)(F)F)c(C(=O)O)s4)CC3(C)C)nc21]

Example 79 was prepared according to general procedure (VII) starting from Example 78 (57 mg). The crude was purified by preparative HPLC (Column B, H₂O+0.1% HCOOH/MeCN+0.1% HCOOH 80:20 to 0:100) and freeze dried with HCl (aq. 0.1N) to obtain Example 78 (30 mg, 30%) as a yellow solid.

¹H-NMR (DMSO-d₆, 300 MHz) δ: 1.35 (s, 6H, (CH₃)₂); 1.53 (s, 6H, (CH₃)₂); 3.77-3.82 (m, 2H, N—CH₂); 3.84-3.89 (m, 4H, 2*N—CH₂); 7.18 (dd, 1H, dd, J 8.7 2.1 Hz, Ar); 7.27 (dd, 1H, J 2.4, 1.8 Hz, Ar); 7.38 (d, 1H, J 8.4, Ar); 7.52-7.58 (m, 2H, 2Ar). COOH and one CH₂ were not observed.

M/Z (M[³⁵Cl]+H)⁺: 612.3.

### Example 80: 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-N,N-dimethylthiazole-4-carboxamide

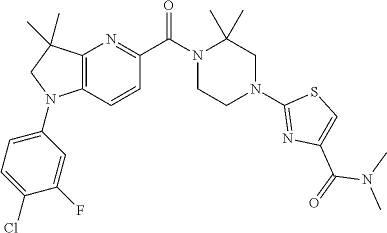 [SMILES: CN(C)C(=O)c1csc(N2CCN(C(=O)c3ccc4c(n3)C(C)(C)CN4c3ccc(Cl)c(F)c3)C(C)(C)C2)n1]

Example 80 was prepared according to general procedure (VIIIb) starting from Example 43 (50 mg), dimethylamine hydrochloride (1.1 eq.), and N,N-diisopropylethylamine (4.0 eq.) in PC. The reaction mixture was directly purified by preparative HPLC (Column A, H₂O+0.1% HCOOH/MeCN+0.1% HCOOH 50:50 to 10:90) and freeze dried with water to obtain Example 80 (37 mg, 71%) as a beige solid.

¹H-NMR (DMSO-d₆, 300 MHz) δ: 1.35 (s, 6H, 2CH₃); 1.52 (s, 6H, 2CH₃); 2.93 (bs, 3H, N—CH₃); 3.11 (bs, 3H, N—CH₃); 3.55-3.58 (m, 2H, CH₂); 3.73 (s, 2H, CH₂); 3.81-3.84 (m, 2H, CH₂); 3.86 (s, 2H, CH₂); 7.11 (s, 1H, Ar); 7.19 (dd, J 8.9, 2.2 Hz, 1H, Ar); 7.33 (dd, J 11.8, 2.6 Hz, 1H, Ar); 7.38 (d, J 8.4 Hz, 1H, Ar); 7.52-7.58 (m, 2H, 2 Ar).

M/Z (M[³⁵Cl]+H)⁺: 572.0.

### Example 81: (1-(4-chloro-3-fluorophenyl)-3,3-dimethyl-2,3-dihydro-1H-pyrrolo[3,2-b]pyridin-5-yl)(4-(4-(4-hydroxypiperidine-1-carbonyl)thiazol-2-yl)-2,2-dimethylpiperazin-1-yl)methanone

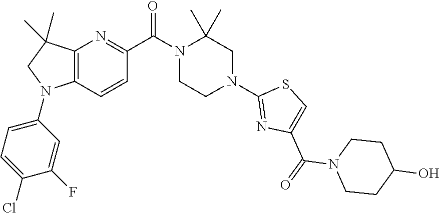 [SMILES: CC1(C)CN(c2ccc(Cl)c(F)c2)c2ccc(C(=O)N3CCN(c4nc(C(=O)N5CCC(O)CC5)cs4)CC3(C)C)nc21]

Example 81 was prepared according to general procedure (VIIIb) starting from Example 43 (50 mg), piperidin-4-ol (1.1 eq.), and N,N-diisopropylethylamine (3.0 eq.) in PC. The reaction mixture was directly purified by preparative HPLC (Column A, H₂O+0.1% HCOOH/MeCN+0.1% HCOOH 60:40 to 20:80) and freeze dried with water to obtain Example 81 (35 mg, 61%) as a beige solid.

¹H-NMR (DMSO-d₆, 300 MHz) δ: 1.35 (s, 8H, 2CH₃+CH₂); 1.52 (s, 6H, 2CH₃); 1.72-1.80 (m, 2H, CH₂); 3.09-3.27 (m, 2H, CH₂); 3.55-3.60 (m, 2H, CH₂); 3.72 (bs, 3H, CH+CH₂); 3.80-3.84 (m, 2H, CH₂); 3.87 (s, 2H, CH₂); 3.92-3.98 (m, 2H, CH₂); 4.76 (d, J 4.2 Hz, 1H, OH); 7.09 (s, 1H, Ar); 7.17-7.21 (m, 1H, Ar); 7.33 (dd, J 11.8, 2.5 Hz, 1H, Ar); 7.39 (d, J 8.4 Hz, 1H, Ar); 7.52-7.58 (m, 2H, 2Ar).

M/Z (M[³⁵Cl]+H)⁺: 628.0.

### Example 82: (1-(4-chloro-3-fluorophenyl)-3,3-dimethyl-2,3-dihydro-1H-pyrrolo[3,2-b]pyridin-5-yl)(2,2-dimethyl-4-(4-(morpholine-4-carbonyl)thiazol-2-yl)piperazin-1-yl)methanone

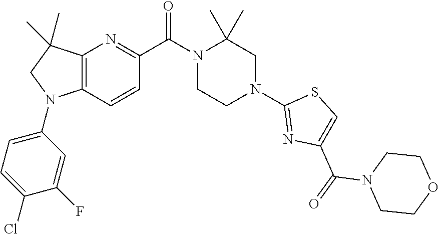 [SMILES: CC1(C)CN(c2ccc(Cl)c(F)c2)c2ccc(C(=O)N3CCN(c4nc(C(=O)N5CCOCC5)cs4)CC3(C)C)nc21]

Example 82 was prepared according to general procedure (VIIIa) starting from Example 43 (50 mg), morpholine (1.1 eq.), and N,N-diisopropylethylamine (3.0 eq.) in PC. The reaction mixture was directly purified by preparative HPLC (Column A, H₂O+0.1% HCOOH/MeCN+0.1% HCOOH 50:50 to 10:90) and freeze dried with water to obtain Example 82 (31 mg, 55%) as a beige solid.

¹H-NMR (DMSO-d₆, 300 MHz) δ: 1.35 (s, 6H, 2CH₃); 1.52 (s, 6H, 2CH₃); 3.55-3.59 (m, 8H, 4CH₂); 3.71 (m, 4H, 2CH₂); 3.80-3.84 (m, 2H, CH₂); 3.86 (s, 2H, CH₂); 7.17-7.21 (m, 2H, 2Ar); 7.33 (dd, J 11.9, 2.6 Hz, 1H, Ar); 7.38 (d, J 8.4 Hz, 1H, Ar); 7.52-7.58 (m, 2H, 2Ar).

M/Z (M[³⁵Cl]+H)⁺: 614.0.

### Example 83: 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)thiazole-4-carboxamide

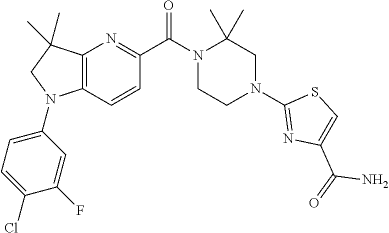 [SMILES: CC1(C)CN(c2ccc(Cl)c(F)c2)c2ccc(C(=O)N3CCN(c4nc(C(N)=O)cs4)CC3(C)C)nc21]

To a solution of Example 43 and ammonia (5.5 eq.), in dioxane (0.1M) was added ((1H-benzo[d][1,2,3]triazol-1-yl)oxy)tris(dimethylamino)phosphonium hexafluorophosphate (1.5 eq.) and the mixture was heated at 80° C. for 16 hours. The reaction mixture was quenched with water (50 mL) and extracted with EtOAc (2*40 mL). The organics were washed with brine (40 mL), dried over MgSO₄ and concentrated under reduced pressure. The residue was purified by flash chromatography (Interchim® 50 μm, DCM 100% to DCM/MeOH 98:02) then further purified by preparative HPLC (Column A, H₂O+0.1% HCOOH/MeCN+0.1% HCOOH 50:50 to 10:90) and freeze dried with water to obtain Example 83 (20 mg, 40%) as a beige solid.

¹H-NMR (DMSO-d₆, 300 MHz) δ: 1.35 (s, 6H, 2CH₃); 1.53 (s, 6H, 2CH₃); 3.52-3.57 (m, 2H, CH₂); 3.80-3.87 (m, 6H, 3CH₂); 7.17-7.21 (m, 1H, Ar); 7.31-7.34 (m, 2H, 2Ar); 7.38 (d, J 8.5 Hz, 1H, Ar); 7.41-7.47 (m, 2H, NH₂); 7.53-7.58 (m, 2H, 2Ar).

M/Z (M[³⁵Cl]+H)⁺: 543.8.

### Compound 117: 5-chloro-1-(3-fluoro-4-methylphenyl)-3,3-dimethyl-2,3-dihydro-1H-pyrrolo[3,2-b]pyridine

Compound 117 was prepared according to general procedure (X) starting from Compound 70 (350 mg) and 4-bromo-2-fluoro-1-methylbenzene (2.0 eq) with ^{t}BuONa (3.0 eq) at 100° C. for 18 hours. The crude was purified by flash chromatography (Interchim®50 μm, CyHex 100% to CyHex/EtOAc 50:50) to obtain Compound 117 (340 mg, 61%) as a beige solid.

M/Z (M[³⁵Cl]+H)⁺: 291.3.

### Example 84: methyl 6-(4-(1-(3-fluoro-4-methylphenyl)-3,3-dimethyl-2,3-dihydro-1H-pyrrolo[3,2-b]pyridine-5-carbonyl)-3,3-dimethylpiperazin-1-yl)-2,4-dimethylnicotinate

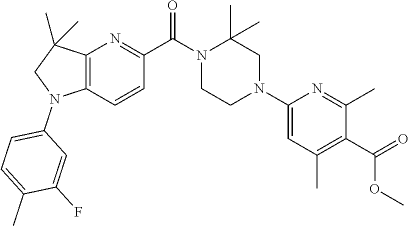 [SMILES: COC(=O)c1c(C)cc(N2CCN(C(=O)c3ccc4c(n3)C(C)(C)CN4c3ccc(C)c(F)c3)C(C)(C)C2)nc1C]

Example 84 was prepared according to general procedure (XIV) starting from Compound 117 (100 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim®50 μm, CyHex 100% to CyHex/EtOAc 20:80) to obtain Example 84 (128 mg, 67%) as a beige solid.

¹H-NMR (CDCl₃, 300 MHz) δ: 1.42 (m, 6H, C(CH₃)₂); 1.62 (s, 6H, C(CH₃)₂); 2.25 (s, 3H, ArCH₃); 2.32 (s, 3H, ArCH₃); 2.55 (bs, 3H, ArCH₃); 3.59-3.67 (m, 2H, N—CH₂); 3.76 (s, 2H, N—CH₂); 3.87 (s, 3H, OCH₃); 3.93-4.06 (m, 4H, 2N—CH₂); 6.10 (s, 1H, Ar); 6.85-6.92 (m, 2H, 2Ar); 7.14-7.20 (m, 1H, Ar); 7.29 (d, J 8.4 Hz, 1H, Ar); 7.47 (d, J 8.4 Hz, 1H, Ar).

M/Z (M+H⁺): 561.0.

### Example 85: 6-(4-(1-(3-fluoro-4-methylphenyl)-3,3-dimethyl-2,3-dihydro-1H-pyrrolo[3,2-b]pyridine-5-carbonyl)-3,3-dimethylpiperazin-1-yl)-2,4-dimethylnicotinic acid

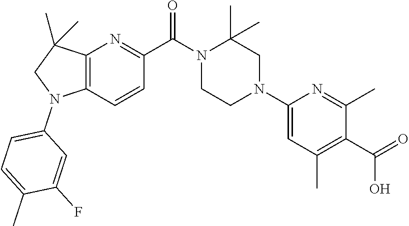 [SMILES: Cc1ccc(N2CC(C)(C)c3nc(C(=O)N4CCN(c5cc(C)c(C(=O)O)c(C)n5)CC4(C)C)ccc32)cc1F]

Example 85 was prepared according to general procedure (XI) starting from Example 84 (125 mg). The crude was purified by preparative HPLC (Column A, H₂O+0.1% HCOOH/MeCN+0.1% HCOOH 70:30 to 30:70) and freeze dried with HCl (0.1N in water) to obtain Example 85 (88 mg, 68%) as a beige solid.

¹H-NMR (DMSO-d₆, 300 MHz) δ: 1.35 (s, 6H, (CH₃)₂); 1.52 (s, 6H, (CH₃)₂); 2.21 (s, 3H, CH₃); 2.34 (s, 3H, CH₃); 3.59-3.72 (m, 2H, NCH₂); 3.83 (s, 2H, NCH₂); 3.92-3.97 (m, 4H, 2*CH₂); 6.61-6.69 (m, 1H, Ar); 7.05-7.09 (m, 2H, 2Ar); 7.26-7.32 (m, 1H, Ar;) 7.35 (d, J 8.4, 1H, Ar); 7.47 (d, J 8.4, 1H, Ar). One CH3 and COOH are not observed.

M/Z (M+H⁺): 547.0.

### Compound 118: 1-((4-chloro-3-fluorophenyl)amino)-2-methylpropan-2-ol

Under inert atmosphere, to a solution of 4-bromo-1-chloro-2-fluorobenzene (1.0 g) and 1-amino-2-methylpropan-2-ol (1.1 eq.) in Dioxane (0.1 M) was added Cesium Carbonate (2 eq.). The mixture was sparged with Argon for 10 min before addition of BrettPhos Pd G4 (0.05 equiv). The reaction mixture was heated at 100° C. for 1 hour. The reaction mixture was filtered through a pad of Celite, the pad was washed with EtOAc (20 mL) then the filtrate was concentrated to dryness. The residue was purified by flash chromatography (Interchim® 50 μm, DCM 100% to DCM/MeOH 99:01) to obtain Compound 118 (710 mg, 68%) as a brown oil.

M/Z (M[³⁵Cl]−OH)⁺: 200.6.

### Compound 119: 2,6-dichloro-N-(4-chloro-3-fluorophenyl)-N-(2-hydroxy-2-methylpropyl)nicotinamide

To a solution of 2,6-dichloronicotinic acid (626 mg) and Compound 118 (1.0 eq.) in DCM (0.1 M) was added pyridine (10 eq) and POCl₃ (2 eq.) at 0° C. The reaction mixture was stirred at 0° C. for 1 hour. The reaction mixture was quenched with HCl (1N aq., 5 mL), diluted with water (15 mL) and extracted with DCM (3*20 mL). The combined organic layers were washed with brine (10 mL), dried over MgSO₄ and concentrated under reduced pressure. The residue was purified by flash chromatography (Interchim® 50 μm, CyHex 100% to CyHex/EtOAc 50:50) to obtain Compound 119 (910 mg, 71%) as a yellow oil.

M/Z (M[³⁵Cl₃]−OH)⁺: 373.6

### Compound 120: 8-chloro-4-(4-chloro-3-fluorophenyl)-2,2-dimethyl-3,4-dihydropyrido[3,2-f][1,4]oxazepin-5(2H)-one

To a solution of Compound 119 (300 mg) in THE (0.1 M) at 0° C. was added Sodium Hydride (60% in mineral oil, 1.1 eq.). The reaction mixture was heated at 50° C. for 21 hours. The reaction mixture was quenched with water (10 mL) and extracted with EtOAc (2*20 mL). The combined organic layers were washed with brine (10 mL) dried over MgSO₄ and concentrated under reduced pressure. The residue was purified by flash chromatography (Interchim® 50 μm, CyHex 100% to CyHex/EtOAc 60:40) to obtain Compound 120 (143 mg, 53%) as a white solid.

M/Z (M[³⁵Cl₂]+H)⁺: 355.6

### Compound 121: 8-chloro-4-(4-chloro-3-fluorophenyl)-2,2-dimethyl-2,3,4,5-tetrahydropyrido[3,2-f][1,4]oxazepine

To a solution of Compound 120 (253 mg) in THE (0.1 M) at 0° C. was added Borane (dimethyl sulfide complexe, 5.0 eq.). The reaction mixture was heated at 50° C. for 1.5 hour. The reaction mixture was quenched with MeOH (3 mL), heated at 40° C. for 20 min and concentrated under reduced pressure. The residue was purified by flash chromatography (Interchim®50 μm, CyHex 100% to CyHex/EtOAc 70:30) to obtain Compound 121 (195 mg, 80%) as a white solid.

M/Z (M[³⁵Cl₂]+H)⁺: 341.1

### Example 86: methyl 6-(4-(4-(4-chloro-3-fluorophenyl)-2,2-dimethyl-2,3,4,5-tetrahydropyrido[3,2-f][1,4]oxazepine-8-carbonyl)-3,3-dimethylpiperazin-1-yl)-2,4-dimethylnicotinate

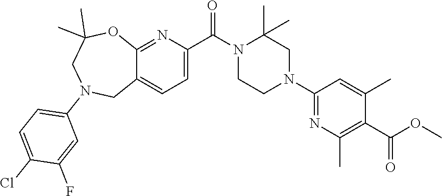 [SMILES: COC(=O)c1c(C)cc(N2CCN(C(=O)c3ccc4c(n3)OC(C)(C)CN(c3ccc(Cl)c(F)c3)C4)C(C)(C)C2)nc1C]

Example 86 was prepared according to general procedure (XIV) starting from Compound 121 (90 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 60:40) to obtain Example 86 (91 mg, 57%) as a yellow oil.

¹H-NMR (DMSO-d₆, 300 MHz) δ: 1.15 (s, 6H, 2CH₃); 1.47 (s, 6H, 2CH₃); 2.22 (s, 3H, CH₃); 2.37 (s, 3H, CH₃); 3.32- 3.39 (m, 2H, N—CH₂); 3.50-3.59 (m, 2H, N—CH₂); 3.78 (s, 3H, O—CH₃); 3.86-3.89 (m, 4H, 2N—CH₂); 4.55 (s, 2H, N—CH₂); 6.32 (s, 1H, Ar); 6.81-6.85 (m, 1H, Ar); 7.00-7.06 (m, 1H, Ar); 7.20-7.28 (m, 2H, 2Ar); 8.17 (d, J 7.7 Hz, 1H, Ar).

M/Z (M[³⁵Cl]+H⁺): 610.9.

### Example 87: 6-(4-(4-(4-chloro-3-fluorophenyl)-2,2-dimethyl-2,3,4,5-tetrahydropyrido[3,2-f][1,4]oxazepine-8-carbonyl)-3,3-dimethylpiperazin-1-yl)-2,4-dimethylnicotinic acid

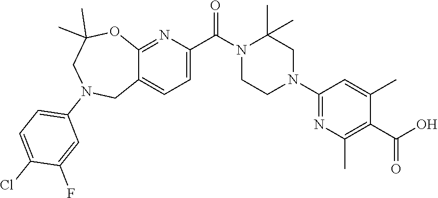 [SMILES: Cc1cc(N2CCN(C(=O)c3ccc4c(n3)OC(C)(C)CN(c3ccc(Cl)c(F)c3)C4)C(C)(C)C2)nc(C)c1C(=O)O]

Example 87 was prepared according to general procedure (XI) starting from Example 86 (91 mg). The crude was purified by preparative HPLC (Column A, H₂O+0.1% HCOOH/MeCN+0.1% HCOOH 70:30 to 30:70) and freeze dried with water to obtain Example 87 (12 mg, 14%) as a white solid.

¹H-NMR (DMSO-d₆, 300 MHz) δ: 1.16 (s, 6H, 2CH₃); 1.47 (s, 6H, 2CH₃); 2.25 (s, 3H, CH₃); 2.36 (s, 3H, CH₃); 3.39- 3.44 (m, 2H, N—CH₂); 3.51-3.57 (m, 2H, N—CH₂); 3.86-3.89 (m, 4H, 2N—CH₂); 4.55 (s, 2H, N—CH₂); 6.30 (s, 1H, Ar); 6.81-6.85 (m, 1H, Ar); 7.01-7.06 (m, 1H, Ar); 7.20-7.28 (m, 2H, 2Ar); 8.17 (d, J 7.7 Hz, 1H, Ar); 12.68 (bs, 1H, COOH).

M/Z (M[³⁵Cl]+H⁺): 596.9.

### Compound 122: 1-(3-chloropyridin-2-yl)-3,3-difluorocyclobutane-1-carbonitrile

Compound 122 was prepared according to general procedure (I) from 3-chloro-2-fluoropyridine (1.0 g) and 3,3-difluorocyclobutane-1-carbonitrile. The crude was purified by flash chromatography (Merck® 60 μm, CyHex 100% to CyHex/EtOAc 80:20) to obtain Compound 122 (1.29 g, 74%) as a clear oil.

M/Z (M[³⁵Cl]+H⁺): 229.6.

### Compound 123: (1-(3-chloropyridin-2-yl)-3,3-difluorocyclobutyl)methanamine

Compound 123 (850 mg, 65%) was obtained as a clear oil following general procedure (XVa), starting from Compound 122 (1.29 g).

M/Z (M[³⁵Cl]+H⁺): 233.4.

### Compound 124: 1′-(4-chloro-3-fluorophenyl)-3,3-difluoro-1′,2′-dihydrospiro[cyclobutane-1,3′-pyrrolo[3,2-b]pyridine]

Compound 124 was prepared according to general procedure (Xc) from Compound 123 (900 mg) and 4-bromo-1-chloro-2-fluorobenzene. The crude was purified by flash chromatography (Interchim® 50 μm, CyHex 100% to CyHex/EtOAc 75:25) to obtain Compound 124 (355 mg, 28%) as a white solid.

M/Z (M[³⁵Cl]+H⁺): 325.6.

### Compound 125: 5′-bromo-1′-(4-chloro-3-fluorophenyl)-3,3-difluoro-1′,2′-dihydrospiro[cyclobutane-1,3′-pyrrolo[3,2-b]pyridine]

Compound 125 (431 mg, 99%) was obtained as an off-white solid following general procedure (XVI) in MeCN without further purification, starting from Compound 124 (350 mg).

M/Z (M[³⁵Cl][⁸¹Br]+H)⁺: 405.7.

### Example 88: methyl 6-(4-(1′-(4-chloro-3-fluorophenyl)-3,3-difluoro-1′,2′-dihydrospiro[cyclobutane-1,3′-pyrrolo[3,2-b]pyridine]-5′-carbonyl)-3,3-dimethylpiperazin-1-yl)-2,4-dimethylnicotinate

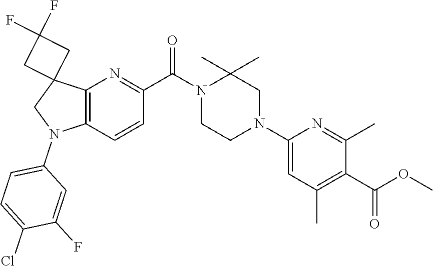 [SMILES: COC(=O)c1c(C)cc(N2CCN(C(=O)c3ccc4c(n3)C3(CN4c4ccc(Cl)c(F)c4)CC(F)(F)C3)C(C)(C)C2)nc1C]

Example 88 was prepared according to general procedure (XIV) starting from Compound 125 (100 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 60:40) to obtain Example 88 (85 mg) as a white solid.

¹H-NMR (CDCl₃, 300 MHz) δ: 1.62 (s, 6H, 2CH₃); 2.32 (s, 3H, CH₃); 2.47 (s, 3H, CH₃); 2.75-2.85 (m, 2H, (CHₐH_{b})₂—CF₂); 3.15-3.30 (m, 2H, (CHₐH_{b})₂-CF₂); 3.65-3.70 (m, 2H, N—CH₂); 3.87 (s, 3H, O—CH₃); 3.98-4.00 (m, 4H, 2N—CH₂); 4.20 (s, 2H, N—CH₂); 6.11 (s, 1H, Ar); 6.91-7.03 (m, 2H, 2Ar); 7.34-7.40 (m, 2H, 2Ar); 7.58 (d, J 8.4 Hz, 1H, Ar).

M/Z (M[³⁵Cl]+H⁺): 628.3.

### Example 89: 6-(4-(1′-(4-chloro-3-fluorophenyl)-3,3-difluoro-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid

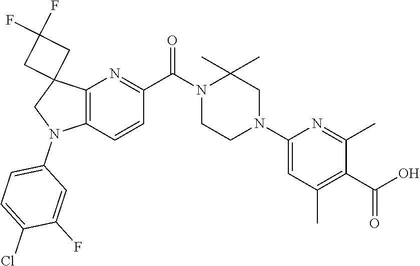 [SMILES: Cc1cc(N2CCN(C(=O)c3ccc4c(n3)C3(CN4c4ccc(Cl)c(F)c4)CC(F)(F)C3)C(C)(C)C2)nc(C)c1C(=O)O]

### Example 90: 6-(4-(1′-(4-chloro-3-fluorophenyl)-3-fluoro-1′,2′-dihydrospiro[cyclobutane-1,3′-pyrrolo[3,2-b]pyridin]-2-en-5′-carbonyl)-3,3-dimethylpiperazin-1-yl)-2,4-dimethylnicotinic acid

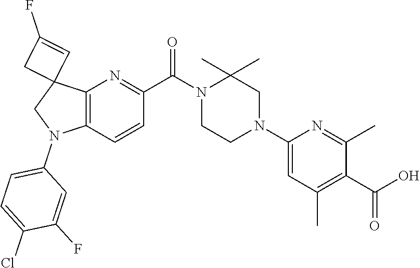 [SMILES: Cc1cc(N2CCN(C(=O)c3ccc4c(n3)C3(C=C(F)C3)CN4c3ccc(Cl)c(F)c3)C(C)(C)C2)nc(C)c1C(=O)O]

Example 89 and Example 90 were prepared according to general procedure (XI) starting from Example 88 (80 mg). The crude was purified by preparative HPLC (Column A, H₂O+0.1% HCOOH/MeCN+0.1% HCOOH 65:35 to 0:100) and the corresponding fractions were freeze dried with HCl (0.1N in water) to obtain Example 89 (35 mg, 22% over 2 steps) as a yellow solid and Example 90 (10 mg, 14% over 2 steps) as a white solid.

### Example 89

¹H-NMR (DMSO-d₆, 300 MHz) δ: 1.51 (s, 6H, (CH₃)₂); 2.30 (s, 3H, CH₃); 2.45 (s, 3H, CH₃); 2.93-3.12 (m, 4H, (CH₂)₂); 3.65-3.69 (m, 2H, CH₂); 3.88-3.94 (m, 4H, (CH₂)₂); 4.33 (s, 2H, CH₂); 6.56 (bs, 1H, Ar); 7.16-7.22 (dd, J 8.7 Hz, 2.7 Hz, 1H, Ar); 7.36 (dd, J 11.7 Hz, 2.7 Hz, 1H, Ar); 7.44 (d, J 8.7 Hz, 1H, Ar); 7.54-7.60 (m, 2H, 2Ar). COOH was not observed.

M/Z (M[³⁵Cl]+H)⁺: 614.5

### Example 90

¹H-NMR (DMSO-d₆, 300 MHz) δ: 1.48 (s, 6H, (CH₃)₂); 2.25 (s, 3H, CH₃); 2.37 (s, 3H, CH₃); 3.01-3.07 (m, 1H, CH); 3.15-3.22 (m, 1H, CH); 3.52-3.58 (m, 2H, CH₂); 3.68 (bs, 2H, CH₂); 3.74-3.79 (m, 2H, CH₂); 4.19-4.33 (m, 2H, CH₂); 5.30 (d, J 8.1 HZ, 1H, CH); 6.27 (bs, 1H, Ar); 7.18-7.22 (m, 1H, Ar); 7.32-7.40 (m, 2H, 2Ar); 7.53-7.61 (m, 2H, 2Ar); 12.65 (bs, 1H, CO₂H).

M/Z (M[³⁵Cl]+H)⁺: 595.0.

### Example 91: methyl 2-((3S,4S)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetate

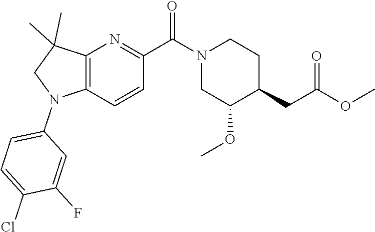 [SMILES: COC(=O)C[C@@H]1CCN(C(=O)c2ccc3c(n2)C(C)(C)CN3c2ccc(Cl)c(F)c2)C[C@H]1OC]

Example 91 was prepared according to general procedure (VIIIb) starting from Compound 68 (100 mg), methyl 2-((3S,4S)-3-methoxypiperidin-4-yl)acetate hydrochloride (1.1 eq.) and triethylamine (3 eq.) in THF. The crude was purified by flash chromatography (Interchim® 50 μm, CyHex 100% to CyHex/EtOAc 0:100) to obtain Example 91 (139 mg, 91%) as a beige solid.

¹H-NMR (CDCl₃, 300 MHz) δ: 1.34 (broad s, 7H, (CH₃)₂, CH); 1.68-2.03 (m, 2H, CH₂); 2.56-2.67 (m, 2H, CH₂); 2.76-3.09 (m, 3H, CH₂, CH); 3.12-3.24 (m, 2H, N—CH₂); 3.58 (s, 3H, O—CH₃); 3.81-3.88 (m, 3H, CH-0+N—CH₂); 3.89-4.01 (m, 0.5H, one rotamer of N—CHₐH_{b}); 4.23-4.40 (m, 0.5H, other rotamer of N—CHₐH_{b}); 4.45-4.60 (m, 0.5H, one rotamer of N—CHₐH_{b}); 4.63-4.76 (m, 0.5H, one rotamer of N—CHₐH_{b}); 7.18 (dd, 8.7 1.8 Hz, 1H, Ar); 7.32 (dd, 2.4, 1.8 Hz, 1H, Ar); 7.33-7.44 (m, 1H, Ar), 7.49-7.58 (m, 2H, 2Ar).

M/Z (M[³⁵Cl]+H)⁺: 490.9

### Example 92: 2-((3S,4S)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetic acid

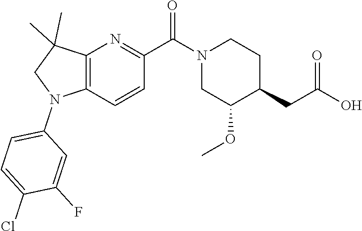 [SMILES: CO[C@@H]1CN(C(=O)c2ccc3c(n2)C(C)(C)CN3c2ccc(Cl)c(F)c2)CC[C@H]1CC(=O)O]

Example 92 was prepared according to general procedure (VII) starting from Example 91 (139 mg). The crude was purified by preparative HPLC (Column A, H₂O+0.1% HCOOH/MeCN+0.1% HCOOH 60:40 to 20:80) then freeze dried with HCl (0.1N in water) to obtain Example 92 (83 mg, 57%) as a yellow solid.

¹H-NMR (DMSO-d₆, 300 MHz) δ: 1.30 (broad s, 7H, (CH₃)₂, CH); 1.68-1.97 (m, 2H, CH₂); 2.07-2.18 (m, 1H, CH); 2.60-2.68 (m, 1H, CH); 2.77-3.01 (m, 3H, CH₂, CH); 3.14-3.33 (s, 3H, rotamer OCH₃); 3.82-3.87 (m, 2H, CH₂); 3.87-3.95 (m, 0.5H, one rotamer of CHₐH_{b}); 4.19-4.29 (m, 0.5H, other rotamer of CHₐH_{b}); 4.36-4.47 (m, 0.5H, one rotamer of CHₐH_{b}; 4.57-4.67 (0.5H, other rotamer of CHₐH_{b}); 7.16 (dd, 8.7 1.8 Hz, 1H, Ar); 7.27 (dd, 2.4, 1.8 Hz, 1H, Ar); 7.33-7.41 (m, 1H, Ar), 7.49-7.56 (m, 2H, 2Ar). COOH was not observed

M/Z (M[³⁵Cl]+H)⁺: 476.3

### Example 93: methyl 2-((3R,4R)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetate

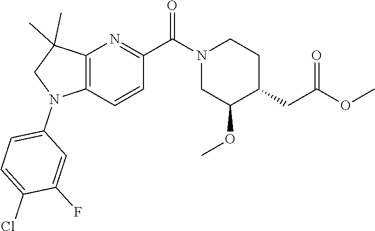 [SMILES: COC(=O)C[C@H]1CCN(C(=O)c2ccc3c(n2)C(C)(C)CN3c2ccc(Cl)c(F)c2)C[C@@H]1OC]

Example 93 was prepared according to general procedure (VIIIb) starting from Compound 68 (100 mg), methyl 2-((3R,4R)-3-methoxypiperidin-4-yl)acetate hydrochloride (1.1 eq.) and triethylamine (3 eq.) in THF. The crude was purified by flash chromatography (Interchim® 50 μm, CyHex 100% to CyHex/EtOAc 0:100) to obtain Example 91 (121 mg, 79%) as a beige solid.

¹H-NMR (CDCl₃, 300 MHz) δ: 1.34 (broad s, 7H, (CH₃)₂, CH); 1.68-2.03 (m, 2H, CH₂); 2.56-2.67 (m, 2H, CH₂); 2.76-3.09 (m, 3H, CH₂, CH); 3.12-3.24 (m, 2H, N—CH₂); 3.58 (s, 3H, O—CH₃); 3.81-3.88 (m, 3H, CH-0+N—CH₂); 3.89-4.01 (m, 0.5H, one rotamer of N—CHₐH_{b}); 4.23-4.40 (m, 0.5H, other rotamer of N—CHₐH_{b}); 4.45-4.60 (m, 0.5H, one rotamer of N—CHₐH_{b}); 4.63-4.76 (m, 0.5H, one rotamer of N—CHₐH_{b}); 7.18 (dd, 8.7 1.8 Hz, 1H, Ar); 7.32 (dd, 2.4, 1.8 Hz, 1H, Ar); 7.33-7.44 (m, 1H, Ar), 7.49-7.58 (m, 2H, 2Ar).

M/Z (M[³⁵Cl]+H)⁺: 490.9

### Example 94: 2-((3R,4R)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetic acid

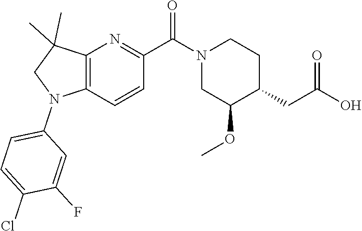 [SMILES: CO[C@H]1CN(C(=O)c2ccc3c(n2)C(C)(C)CN3c2ccc(Cl)c(F)c2)CC[C@@H]1CC(=O)O]

Example 94 was prepared according to general procedure (VII) starting from Example 93 (121 mg). The crude was purified by preparative HPLC (Column A, H₂O+0.1% HCOOH/MeCN+0.1% HCOOH 60:40 to 20:80) then freeze dried with HCl (0.1N in water) to obtain Example 94 (77 mg, 61%) as a yellow solid.

¹H-NMR (DMSO-d₆, 300 MHz) δ: 1.32 (broad s, 7H, (CH₃)₂, CH); 1.70-1.96 (m, 2H, CH₂); 2.05-2.18 (m, 1H, CH); 2.57-2.67 (m, 1H, CH); 2.78-3.10 (m, 3H, CH, CH₂); 3.16-3.34 (2 d, 3H, rotamer OCH₃); 3.84 (s, 2H, CH₂); 3.89-3.97 (m, 0.5H, one rotamer of CHₐH_{b}); 4.21-4.31 (m, 0.5H, other rotamer of CHₐH_{b}); 4.39-4.51 (m, 0.5H, one rotamer of CHₐH_{b}); 4.59-4.72 (0.5H, other rotamer of CHₐH_{b}); 7.17 (dd, J 8.7 2.1 Hz, 1H, Ar); 7.27 (dd, J 2.4, 1.8 Hz, 1H, Ar); 7.28-7.43 (m, 2H, 2Ar), 7.50-7.58 (m, 2H, 2Ar). COOH was not observed

M/Z (M[³⁵Cl]+H)⁺: 476.3

### Example 95: ethyl (2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)thiazole-4-carbonyl)glycinate

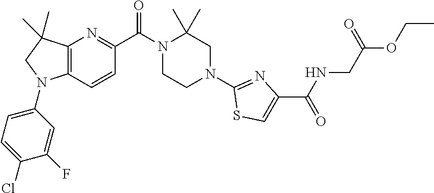 [SMILES: CCOC(=O)CNC(=O)c1csc(N2CCN(C(=O)c3ccc4c(n3)C(C)(C)CN4c3ccc(Cl)c(F)c3)C(C)(C)C2)n1]

Example 95 was prepared according to general procedure (VIIIa) starting from Example 43 (75 mg), ethyl glycinate hydrochloride (1.1 eq.), and N,N-diisopropylethylamine (3.0 eq.) in PC. The crude was purified by flash chromatography (Interchim® 50 μm, DCM 100% to DCM/MeOH 98:02) to obtain Example 95 (80 mg, 92%) as an orange solid.

¹H-NMR (DMSO-d₆, 300 MHz) δ: 1.20 (t, 3H, J 7.8 Hz, CH₂CH₃); 1.36 (s, 6H, 2CH₃); 1.54 (s, 6H, 2CH₃); 3.55-3.59 (m, 2H, N—CH₂); 3.83-3.87 (m, 6H, 3N—CH₂); 3.96-3.98 (m, 2H, N—CH₂); 4.12 (q, 2H, J 7.8 Hz, CH₂CH₃); 7.17-7.21 (m, 1H, Ar); 7.30-7.40 (m, 3H, 3Ar); 7.52-7.58 (m, 2H, 2Ar), 8.37-8.42 (m, 1H, NH).

M/Z (M[³⁵Cl]+H)⁺: 630.0

### Example 96: (2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)thiazole-4-carbonyl)glycine

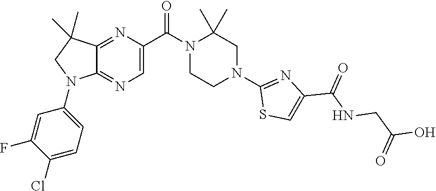 [SMILES: CC1(C)CN(c2ccc(Cl)c(F)c2)c2ncc(C(=O)N3CCN(c4nc(C(=O)NCC(=O)O)cs4)CC3(C)C)nc21]

Example 96 was prepared according to general procedure (VII) starting from Example 95 (80 mg). The crude was suspended in DMSO (2 mL) and precipitated in water (40 mL). The precipitate was recovered by filtration and freeze dried in HCl (0.1N in water) to obtain Example 96 (43 mg, 53%) as a yellow solid.

¹H-NMR (DMSO-d₆, 300 MHz) δ: 1.35 (s, 6H, 2CH₃); 1.54 (s, 6H, 2CH₃); 3.59-3.66 (m, 2H, CH₂); 3.82-3.91 (m, 8H, 4CH₂); 7.17-7.21 (m, 1H, Ar); 7.33 (dd, J 11.9, 2.6 Hz, 1H, Ar); 7.37-7.40 (m, 2H, 2Ar); 7.53-7.59 (m, 2H, 2Ar); 8.28-8.32 (m, 1H, NH). COOH was not observed.

M/Z (M[³⁵Cl]+H)⁺: 602.0.

### Example 97: methyl 2-((3S,4R)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetate

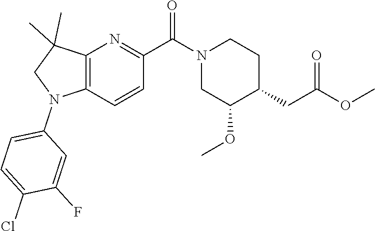 [SMILES: COC(=O)C[C@H]1CCN(C(=O)c2ccc3c(n2)C(C)(C)CN3c2ccc(Cl)c(F)c2)C[C@H]1OC]

Example 97 was prepared according to general procedure (VIIIb) starting from Compound 68 (100 mg), methyl 2-((3S,4R)-3-methoxypiperidin-4-yl)acetate hydrochloride (1.1 eq.) and triethylamine (3 eq.) in THF. The crude was purified by flash chromatography (Interchim® 50 μm, CyHex 100% to CyHex/EtOAc 0:100) to obtain Example 91 (135 mg) as a beige solid.

¹H-NMR (CDCl₃, 300 MHz) δ: 1.38-1.57 (m, 7H, (CH₃)₂+N—CH₂CHₐH_{b}); 1.74-1.78 (m, 1H, N—CH₂CHₐH_{b}); 2.19-2.38 (m, 2H, CH₂—CO); 2.51-2.61 (m, 1H, CH); 2.89-2.97 (m, 2H, 2N—CHₐH_{b}); 3.20-3.25 (m, 1H, CH—OCH₃); 3.38-3.42 (m, 2H, N—CH₂); 3.62-3.65 (m, 3H, O—CH₃); 3.67-3.72 (m, 3H, COOCH₃); 4.14-4.22 (m, 0.5H, one rotamer of N—CHₐH_{b}); 4.49-4.60 (m, 1H, N—CHₐH_{b}); 4.82-4.94 (m, 0.5H, one rotamer of N—CHₐH_{b}); 6.91-7.05 (m, 2H, 2Ar); 7.31-7.41 (m, 2H, 2Ar); 7.47-7.61 (m, 1H, Ar).

M/Z (M[³⁵Cl]+H)⁺: 490.3

### Example 98: 2-((3S,4R)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetic acid

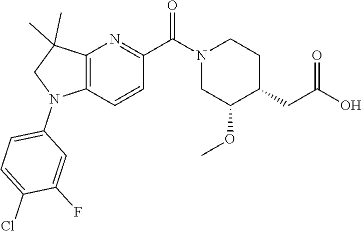 [SMILES: CO[C@@H]1CN(C(=O)c2ccc3c(n2)C(C)(C)CN3c2ccc(Cl)c(F)c2)CC[C@@H]1CC(=O)O]

Example 98 was prepared according to general procedure (VII) starting from Example 97 (121 mg). The crude was purified by preparative HPLC (Column A, H₂O+0.1% HCOOH/MeCN+0.1% HCOOH 60:40 to 20:80) then freeze dried with HCl (0.1N in water) to obtain Example 98 (66 mg, 47% over 2 steps) as a yellow solid.

¹H-NMR (DMSO-d₆, 300 MHz) δ: 1.31-1.58 (m, 8H, (CH₃)₂, CH₂); 2.04-2.23 (m, 2H, CH₂); 2.30-2.41 (m, 1H, CH); 2.78-2.94 (m, 2.5H, CH₂ and one rotamer of CHₐH_{b}); 3.06-3.46 (m, 3.5H, CH₃ other rotamer of CHₐH_{b}); 3.79-3.91 (m, 2H, CH₂); 3.96-4.10 (m, 0.5H, one rotamer of CHₐH_{b}); 4.26-4.47 (m, 2*0.5H, other rotamer of CHₐH_{b} and one rotamer of CHₐH_{b}); 4.62-4.74 (0.5H, other rotamer of CH)ₐH_{b}; 7.17 (dd, J 9.0, 2.1 Hz, 1H, Ar); 7.2-7.39 (m, 2H, 2Ar); 7.51-7.59 (m, 2H, 2Ar). COOH was not observed

M/Z (M[³⁵Cl]+H)⁺: 476.3

### Compound 126: 3-chloro-N-(4-chloro-3-fluorophenyl)-N-(2-methylallyl)pyrazin-2-amine

Crude Compound 126 (10.0 g) was obtained as a brown oil according to general procedure (XVII) on a 5.0 g scale, using 4-chloro-3-fluoroaniline.

M/Z (M[³⁵Cl]₂+H)⁺: 312.2.

### Compound 127: 5-(4-chloro-3-fluorophenyl)-7,7-dimethyl-6,7-dihydro-5H-pyrrolo[2,3-b]pyrazine

Compound 127 was prepared according to general procedure (XVIII) starting from crude Compound 126 (10.0 g).

The crude was precipitated from a water/ethanol mixture (1:2, about 4 V) to obtain Compound 127 (3.32 g, 36% over 2 steps) as a beige solid.

M/Z (M[³⁵Cl]+H)⁺: 278.0.

### Compound 128: 2-bromo-5-(4-chloro-3-fluorophenyl)-7,7-dimethyl-6,7-dihydro-5H-pyrrolo[2,3-b]pyrazine

Compound 128 (4.20 g, 99%) was obtained as an off-white solid following general procedure (XVI) in MeCN without further purification, starting from Compound 127 (3.32 g).

M/Z (M[⁸¹Br³⁵Cl]+H)⁺: 358.1.

### Example 99: methyl 6-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinate

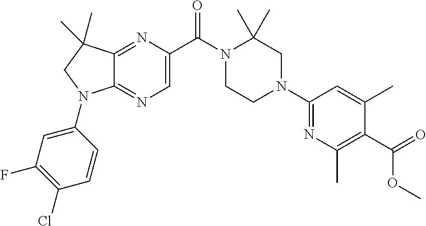 [SMILES: COC(=O)c1c(C)cc(N2CCN(C(=O)c3cnc4c(n3)C(C)(C)CN4c3ccc(Cl)c(F)c3)C(C)(C)C2)nc1C]

Example 99 was prepared according to general procedure (XIV) starting from Compound 128 (1.0 g) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 50:50) to obtain Example 99 (0.82 g, 51%) as a yellow solid.

¹H-NMR (DMSO-d₆, 300 MHz) δ: 1.40 (s, 6H, 2CH₃); 1.50 (s, 6H, 2CH₃); 2.24 (s, 3H, CH₃); 2.35 (s, 3H, CH₃); 3.53- 3.57 (m, 2H, N—CH₂—CH₂—N); 3.77-3.84 (m, 5H, N—CH₂—CH₂—N+O—CH₃); 3.88 (s, 2H, N—CH₂); 3.99 (s, 2H, N—CH₂); 6.37 (s, 1H, Ar); 7.62 (t, J 8.7 Hz, 1H, Ar); 7.73 (dd, J 8.7, 2.3 Hz, 1H, Ar); 8.09 (dd, J 12.4, 2.3 Hz, 1H, Ar); 8.22 (s, 1H, Ar).

M/Z (M[³⁵Cl]+H)⁺: 581.4.

### Example 100: 6-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid

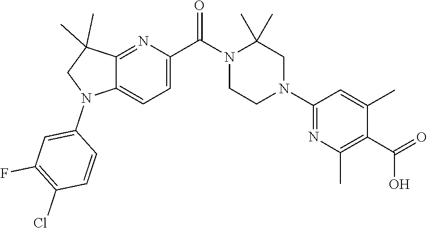 [SMILES: Cc1cc(N2CCN(C(=O)c3ccc4c(n3)C(C)(C)CN4c3ccc(Cl)c(F)c3)C(C)(C)C2)nc(C)c1C(=O)O]

Example 100 was prepared according to general procedure (XI) starting from Example 99 (824 mg). The reaction mixture was quenched in water (50 mL), washed with Et₂O (2*40 mL), acidified with HCl (pH=5) and extracted with DCM (2*50 mL). The combined DCM layers were washed with brine (50 mL), dried over MgSO₄, and concentrated under reduced pressure. To the residue was added DCM (20 mL) and the solid was recovered by filtration to obtain Example 100 (480 mg, 60%) as a white solid.

¹H-NMR (DMSO-d₆, 300 MHz) δ: 1.40 (s, 6H, CH₃×2); 1.50 (s, 6H, CH₃×2); 2.27 (s, 3H, CH₃); 2.37 (s, 3H, CH₃); 3.52-3.55 (m, 2H, N—CH₂—CH₂—N); 3.79-3.82 (m, 2H, N—CH₂—CH₂—N); 3.88 (s, 2H, CH₂); 3.99 (s, 2H, CH₂); 6.35 (s, 1H, Ar); 7.62 (t, J 8.7 Hz, 1H, Ar); 7.73 (dd, J 8.7, 2.3 Hz, 1H, Ar); 8.09 (dd, J 12.4, 2.3 Hz, 1H, Ar); 8.22 (s, 1H, Ar), 12.70 (bs, 1H, COOH).

M/Z (M[³⁵Cl]+H)⁺: 567.4.

### Example 101: (1-(4-chloro-3-fluorophenyl)-3,3-dimethyl-2,3-dihydro-1H-pyrrolo[3,2-b]pyridin-5-yl)(2,2-dimethyl-4-(4-(4-methylpiperazine-1-carbonyl)thiazol-2-yl)piperazin-1-yl)methanone hydrochloride

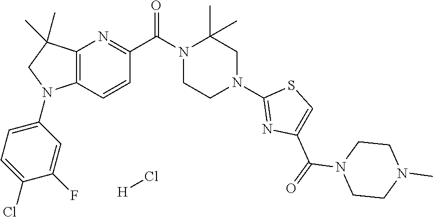 [SMILES: CN1CCN(C(=O)c2csc(N3CCN(C(=O)c4ccc5c(n4)C(C)(C)CN5c4ccc(Cl)c(F)c4)C(C)(C)C3)n2)CC1 ; Cl]

Example 101 was prepared according to general procedure (VIIIa) starting from Example 43 (50 mg), 1-methylpiperazine (1.1 eq.), and N,N-diisopropylethylamine (3.0 eq.) in PC. The crude was purified by preparative HPLC (Column A, H₂O+0.1% HCOOH/MeCN+0.1% HCOOH 70:30 to 30:70) then freeze dried with HCl (0.1N in water) to obtain Example 101 (7 mg, 12%) as a yellow solid.

¹H-NMR (DMSO-d₆, 300 MHz) δ: 1.35 (s, 6H, 2CH₃); 1.53 (s, 6H, 2CH₃); 2.79 (s, 1.5H, one rotamer of CH₃); 2.80 (s, 1.5H, other rotamer of CH₃); 3.00-3.12 (m, 3H, CH₂+ one rotamer of CH₂); 3.43-3.48 (m, 5H, 2CH₂+other rotamer of CH₂); 3.73 (s, 2H, CH₂); 3.82-3.85 (m, 2H, CH₂); 3.87 (s, 2H, CH₂); 4.49-4.77 (m, 2H, CH₂) 7.17-7.21 (m, 1H, Ar); 7.31-7.36 (m, 2H, 2Ar); 7.39 (d, J 8.4 Hz, 1H, Ar); 7.53-7.59 (m, 2H, 2Ar).

M/Z (M[³⁵Cl]+H)⁺: 626.9.

### Compound 129: 2-(3-chloropyridin-2-yl)-2-ethylbutanenitrile

Compound 129 was prepared according to general procedure (I) from 3-chloro-2-fluoropyridine (1.0 g) and 2-ethylbutanenitrile. The crude was purified by flash chromatography (Merck® 60 μm, CyHex 100% to CyHex/EtOAc 80:20) to obtain Compound 129 (1.31 g, 83%) as a clear oil.

M/Z (M[³⁵Cl]+H⁺): 209.6.

### Compound 130: 2-(3-chloropyridin-2-yl)-2-ethylbutan-1-amine

Compound 130 (0.92 g, 69%) was obtained as a clear oil following general procedure (XVa), starting from Compound 129 (1.30 g).

M/Z (M[³⁵Cl]+H⁺): 213.6.

### Compound 131: 1-(4-chloro-3-fluorophenyl)-3,3-diethyl-2,3-dihydro-1H-pyrrolo[3,2-b]pyridine

Compound 131 was prepared according to general procedure (Xc) from Compound 130 (900 mg) and 4-bromo-1-chloro-2-fluorobenzene. The crude was purified by flash chromatography (Interchim® 50 μm, CyHex 100% to CyHex/EtOAc 75:25) to obtain Compound 131 (644 mg, 50%) as a yellow oil.

M/Z (M[³⁵Cl]+H⁺): 305.7.

### Compound 132: 5-bromo-1-(4-chloro-3-fluorophenyl)-3,3-diethyl-2,3-dihydro-1H-pyrrolo[3,2-b]pyridine

Compound 132 was prepared according to general procedure (XVI) in MeCN from Compound 131 (630 mg). The crude was purified by flash chromatography (Interchim® 50 μm, CyHex 100% to CyHex/EtOAc 90:10) to obtain Compound 132 (623 mg, 79%) as a brown oil.

M/Z (M[³⁵Cl][⁸¹Br]+H)⁺: 385.7.

### Example 102: methyl 6-(4-(1-(4-chloro-3-fluorophenyl)-3,3-diethyl-2,3-dihydro-1H-pyrrolo[3,2-b]pyridine-5-carbonyl)-3,3-dimethylpiperazin-1-yl)-2,4-dimethylnicotinate

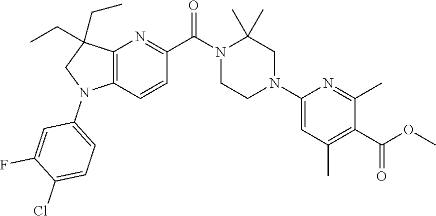 [SMILES: CCC1(CC)CN(c2ccc(Cl)c(F)c2)c2ccc(C(=O)N3CCN(c4cc(C)c(C(=O)OC)c(C)n4)CC3(C)C)nc21]

Example 102 was prepared according to general procedure (XIV) starting from Compound 132 (100 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 50:50) to obtain Example 102 (82 mg) as a yellow solid.

M/Z (M[³⁵Cl]+H⁺): 608.4.

### Example 103: 6-(4-(1-(4-chloro-3-fluorophenyl)-3,3-diethyl-2,3-dihydro-1H-pyrrolo[3,2-b]pyridine-5-carbonyl)-3,3-dimethylpiperazin-1-yl)-2,4-dimethylnicotinic acid

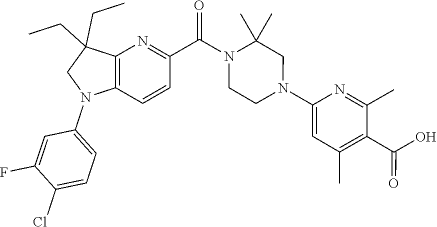 [SMILES: CCC1(CC)CN(c2ccc(Cl)c(F)c2)c2ccc(C(=O)N3CCN(c4cc(C)c(C(=O)O)c(C)n4)CC3(C)C)nc21]

Example 103 was prepared according to general procedure (XI) starting from Example 102 (80 mg). The crude was purified by preparative HPLC (Column A, H₂O+0.1% HCOOH/MeCN+0.1% HCOOH 65:35 to 0:100) and freeze dried with HCl (0.1N in water) to obtain Example 103 (17 mg, 11% over 2 steps) as a yellow solid.

¹H-NMR (DMSO-d₆, 300 MHz) δ: 0.76 (t, J 7.2 Hz, 6H, (CH₂CH₃)₂); 1.52 (s, 6H, (CH₃)₂); 1.73 (q, 4H, J 7.2 Hz (CH₂CH₃)₂); 2.33 (s, 3H, CH₃); 3.62-3.69 (m, 2H, CH₂); 3.84-3.94 (m, 6H, (CH₂)₃); 6.77 (bs, 1H, Ar); 7.22 (dd, J 9.0, 2.1 Hz, 1H, Ar); 7.33-7.39 (m, 2H, 2Ar); 7.51-7.57 (m, 2H, 2Ar). COOH and one CH₃ were not observed.

M/Z (M[³⁵Cl]+H)⁺: 594.9.

### Compound 133: tert-butyl 4-(5-(2-methoxy-2-oxoethyl)pyridin-2-yl)-2,2-dimethylpiperazine-1-carboxylate

Compound 133 was prepared according to general procedure (Xb), starting from methyl 2-chloroisonicotinate (200 mg) and tert-butyl 2,2-dimethylpiperazine-1-carboxylate (1.2 eq.). The crude was directly engaged in the following step.

M/Z (M+H)⁺:350.8

### Compound 134: methyl 2-(3,3-dimethylpiperazin-1-yl)isonicotinate

Compound 134 was obtained as a brown solid (261 mg, 78% over 2 steps) according to general procedure (XII) starting from Compound 133 in DCM using TFA.

M/Z (M+H)⁺:250.7

### Example 104: methyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)isonicotinate

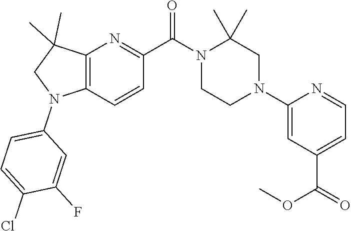 [SMILES: COC(=O)c1ccnc(N2CCN(C(=O)c3ccc4c(n3)C(C)(C)CN4c3ccc(Cl)c(F)c3)C(C)(C)C2)c1]

Example 104 was prepared according to general procedure (VIIIb) starting from Compound 68 (75 mg), Compound 134 (1.1 eq.), and N,N-diisopropylethylamine (3.0 eq.) in THF. The crude was purified by flash chromatography (Interchim® 50 μm, CyHex 100% to CyHex/EtOAc 50:50) to obtain Example 104 (95 mg, 74%) as a beige solid.

¹H-NMR (DMSO-d₆+D₂O, 300 MHz) δ: 1.36 (s, 6H, C(CH₃)₂); 1.51 (s, 6H, C(CH₃)₂); 3.57-3.60 (m, 2H, N—CH₂); 3.81-3.94 (m, 9H, 3N—CH₂+O—CH₃); 7.00-7.04 (m, 2H, 2Ar); 7.18 (dd, J 8.9, 2.0 Hz, 1H, Ar); 7.30-7.38 (m, 2H, 2Ar); 7.52-7.58 (m, 2H, 2Ar); 8.26 (d, J 5.7 Hz, 1H).

M/Z (M[³⁵Cl]+H)⁺: 553.0.

### Example 105: 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)isonicotinic acid

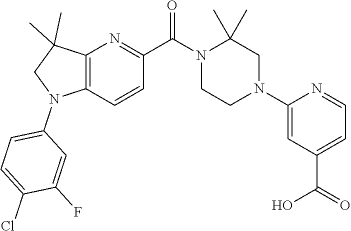 [SMILES: CC1(C)CN(c2ccc(Cl)c(F)c2)c2ccc(C(=O)N3CCN(c4cc(C(=O)O)ccn4)CC3(C)C)nc21]

Example 105 was prepared according to general procedure (VII) starting from Example 104 (95 mg). The crude was purified by preparative HPLC (Column A, H₂O+0.1% HCOOH/MeCN+0.1% HCOOH 60:40 to 20:80) freeze dried with HCl (0.1N in water) to obtain Example 105 (80 mg, 81%) as a yellow solid.

¹H-NMR (DMSO-d₆+D₂O, 300 MHz) δ: 1.35 (s, 6H, C(CH₃)₂); 1.53 (s, 6H, C(CH₃)₂); 3.65-3.67 (m, 2H, N—CH₂); 3.85 (s, 2H, N—CH₂); 3.88-3.91 (m, 2H, N—CH₂); 3.95 (s, 2H, CH₂), 7.11 (dd, J 5.7, 0.7 Hz, 1H, Ar); 7.18 (dd, J 8.9, 2.0 Hz, 1H, Ar); 7.29-7.39 (m, 3H, 3Ar); 7.52-7.58 (m, 2H, 2Ar); 8.19 (d, J 5.7 Hz, 1H). COOH was not observed.

M/Z (M[³⁵Cl]+H)⁺: 538.9.

### Compound 135: tert-butyl 4-(4-(ethoxycarbonyl)pyridin-3-yl)-2,2-dimethylpiperazine-1-carboxylate

Compound 135 was prepared according to general procedure (Xb), starting from ethyl 3-chloroisonicotinate (217 mg) and tert-butyl 2,2-dimethylpiperazine-1-carboxylate (1.2 eq.). The crude was directly engaged in the following step.

M/Z (M+H)⁺: 364.8

### Compound 136: ethyl 3-(3,3-dimethylpiperazin-1-yl)isonicotinate

Compound 136 was obtained as a brown solid (209 mg, 60% over 2 steps) according to general procedure (XII) starting from Compound 135 (261 mg) in DCM using TFA.

M/Z (M+H)⁺: 264.7

### Example 106: ethyl 3-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)isonicotinate

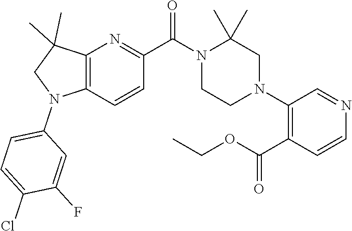 [SMILES: CCOC(=O)c1ccncc1N1CCN(C(=O)c2ccc3c(n2)C(C)(C)CN3c2ccc(Cl)c(F)c2)C(C)(C)C1]

Example 106 was prepared according to general procedure (VIIIb) starting from Compound 68 (75 mg), Compound 136 (1.1 eq.), and N,N-diisopropylethylamine (3.0 eq.) in THF. The crude was purified by flash chromatography (Interchim® 50 μm, CyHex 100% to CyHex/EtOAc 0:100) to obtain Example 106 (65 mg, 50%) as a beige solid.

¹H-NMR (DMSO-d₆, 300 MHz) δ: 1.28-1.33 (m, 9H, CH₂CH₃+C(CH₃)₂); 1.55 (s, 6H, C(CH₃)₂); 3.23-3.31 (m, 2H, N—CH₂); 3.33-3.38 (m, 2H, N—CH₂); 3.71-3.74 (m, 2H, N—CH₂); 3.85 (s, 2H, N—CH₂); 4.31 (q, 2H, 7.2 Hz, CH₂CH₃); 7.18 (dd, J 8.9, 2.1 Hz, 1H, Ar); 7.35-7.40 (m, 2H, 2Ar); 7.52-7.58 (m, 2H, 2Ar); 8.09 (d, J 5.2 Hz, 1H, Ar); 8.43 (s, 1H, Ar).

M/Z (M[³⁵Cl]+H)⁺: 567.0.

### Example 107: 3-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)isonicotinic acid

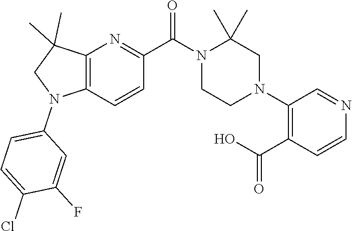 [SMILES: CC1(C)CN(c2ccc(Cl)c(F)c2)c2ccc(C(=O)N3CCN(c4cnccc4C(=O)O)CC3(C)C)nc21]

Example 107 was prepared according to general procedure (VII) starting from Example 106 (65 mg). The crude was purified by preparative HPLC (Column A, H₂O+0.1% HCOOH/MeCN+0.1% HCOOH 70:30 to 30:70) and freeze dried with HCl (0.1N in water) to obtain Example 107 (45 mg, 68%) as a yellow solid.

¹H-NMR (DMSO-d₆, 300 MHz) δ: 1.34 (s, 6H, C(CH₃)₂); 1.55 (s, 6H, C(CH₃)₂); 3.38-3.46 (m, 4H, 2CH₂); 3.75-3.81 (m, 2H, CH₂); 3.85 (s, 2H, CH₂), 7.18 (dd, J 8.9, 2.1 Hz, 1H, Ar); 7.32 (dd, J 11.9, 2.5 Hz, 1H, Ar); 7.38 (d, J 8.4 Hz, 1H, Ar); 7.55 (m, 2H, 2Ar); 7.73 (d, J 5.3 Hz, 1H, Ar), 8.19 (d, J 5.2 Hz, 1H, Ar); 8.51 (s, 1H, Ar).

M/Z (M[³⁵Cl]+H)⁺: 538.9.

### Compound 137: ethyl 6-((1-((benzyloxy)carbonyl)piperidin-3-yl)amino)nicotinate

Compound 137 was prepared according to general procedure (Xb), starting from ethyl 6-chloronicotinate (300 mg) and benzyl 3-aminopiperidine-1-carboxylate (1.2 eq.). The crude was purified by flash chromatography (Interchim® 50 μm, CyHex 100% to CyHex/EtOAc 60:40) to obtain Compound 137 (330 mg, 53%) as a yellow oil.

M/Z (M+H)⁺: 384.9

### Compound 138: ethyl 6-(piperidin-3-ylamino)nicotinate

A solution of of Compound 137 (330 mg) in EtOH (8.6 mL) was sparged with Argon for 5 minutes then Palladium on Charcoal (10% w/w, 0.1 eq) was added. The reaction mixture was stirred under a hydrogen atmosphere (1 Bar) for 16 hours, then filtered over a Celite pad which was rinsed with EtOAc (50 mL). The filtrate was concentrated under reduced pressure to obtain Compound 138 (207 mg, 97%) as a yellow oil.

M/Z (M+H)⁺: 250.7.

### Example 108: ethyl 6-((1-(1-(4-chloro-3-fluorophenyl)-3,3-dimethyl-2,3-dihydro-1H-pyrrolo[3,2-b]pyridine-5-carbonyl)piperidin-3-yl)amino)nicotinate

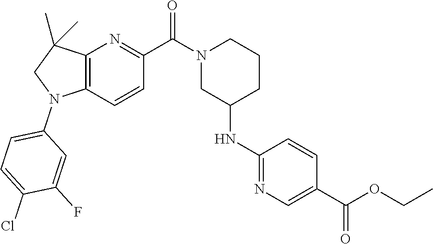 [SMILES: CCOC(=O)c1ccc(NC2CCCN(C(=O)c3ccc4c(n3)C(C)(C)CN4c3ccc(Cl)c(F)c3)C2)nc1]

Example 108 was prepared according to general procedure (VIIIb) starting from Compound 68 (75 mg), Compound 138 (1.1 eq.), and N,N-diisopropylethylamine (3.0 eq.) in MeTHF to obtain crude Example 108 (133 mg) as a beige solid.

M/Z (M[³⁵Cl]+H)⁺: 553.0.

### Example 109: 6-((1-(1-(4-chloro-3-fluorophenyl)-3,3-dimethyl-2,3-dihydro-1H-pyrrolo[3,2-b]pyridine-5-carbonyl)piperidin-3-yl)amino)nicotinic acid

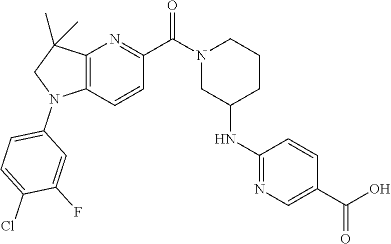 [SMILES: CC1(C)CN(c2ccc(Cl)c(F)c2)c2ccc(C(=O)N3CCCC(Nc4ccc(C(=O)O)cn4)C3)nc21]

Example 109 was prepared according to general procedure (VII) starting from Example 108 (187 μmol). The crude was purified by preparative HPLC (Column A, H₂O+0.1% HCOOH/MeCN+0.1% HCOOH 70:30 to 30:70) and freeze dried with water to obtain Example 109 (73 mg, 75% over 2 steps) as a white solid.

¹H-NMR (DMSO-d₆, 300 MHz) δ: 1.23-1.27 (m, 6H, 2CH₃); 1.56-1.70 (m, 2H, CH₂); 1.78-1.86 (m, 1H, CHₐH_{b}); 1.98-2.04 (m, 1H, CHₐH_{b}); 3.37-3.46 (m, 2H, CH₂); 3.71-3.78 (m, 3H, CH₂+CHₐH_{b}); 3.88-3.95 (m, 1H, CHₐH_{b}); 3.97-4.09 (m, 1H, CH); 6.53-6.55 (d, J 8.1 Hz, 1H, Ar); 7.07-7.09 (m, 1H, Ar); 7.13-7.16 (m, 1H, Ar); 7.27-7.29 (m, 1H, Ar); 7.33-7.35 (m, 1H, Ar); 7.44-7.49 (m, 1H, Ar); 7.79 (d, J 8.3 Hz, 1H, Ar); 8.37 (bs, 1H, Ar). COOH and NH were not observed.

M/Z (M[³⁵Cl]+H)⁺: 524.9.

### Compound 139: ethyl 6-((1-(tert-butoxycarbonyl)piperidin-3-yl)(methyl)amino)nicotinate

Compound 139 was prepared according to general procedure (Xb), starting from ethyl 6-chloronicotinate (300 mg) and tert-butyl 3-(methylamino)piperidine-1-carboxylate (1.2 eq.) to obtain crude Compound 139 (937 mg) as a brown oil.

M/Z (M+H)⁺: 364.8

### Compound 140: ethyl 6-(methyl(piperidin-3-yl)amino)nicotinate

Compound 140 was prepared according to general procedure (XII) starting from Compound 139 (1.62 mmol) in DCM using HCl in Et₂O. The crude was purified by flash chromatography (Biotage*KPNH, CyHex 100% to EtOAc 100%) to obtain Compound 140 (44 mg, 10% over 2 steps) as a yellow oil.

M/Z (M+H)⁺: 264.7

### Example 110: ethyl 6-((1-(1-(4-chloro-3-fluorophenyl)-3,3-dimethyl-2,3-dihydro-1H-pyrrolo[3,2-b]pyridine-5-carbonyl)piperidin-3-yl)(methyl)amino)nicotinate

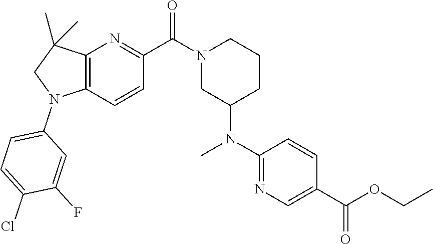 [SMILES: CCOC(=O)c1ccc(N(C)C2CCCN(C(=O)c3ccc4c(n3)C(C)(C)CN4c3ccc(Cl)c(F)c3)C2)nc1]

Example 110 was prepared according to general procedure (VIIIb) starting from Compound 68 (49 mg), Compound 140 (1.1 eq.), and N,N-diisopropylethylamine (3.0 eq.) in MeTHF. The crude was purified by flash chromatography (Interchim® 50 μm, CyHex 100% to CyHex/EtOAc 50/50) to obtain Example 110 (73 mg, 84%) as a yellow solid.

M/Z (M[³⁵Cl]+H)⁺: 567.0.

### Example 111: 6-((1-(1-(4-chloro-3-fluorophenyl)-3,3-dimethyl-2,3-dihydro-1H-pyrrolo[3,2-b]pyridine-5-carbonyl)piperidin-3-yl)(methyl)amino)nicotinic acid hydrochloride

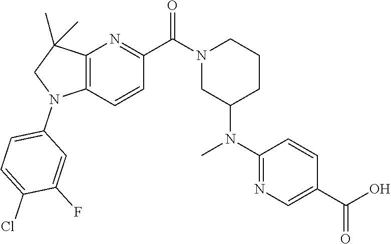 [SMILES: CN(c1ccc(C(=O)O)cn1)C1CCCN(C(=O)c2ccc3c(n2)C(C)(C)CN3c2ccc(Cl)c(F)c2)C1]

Example 111 was prepared according to general procedure (VIII) starting from Example 110 (73 mg). The crude was purified by preparative HPLC (Column A, H₂O+0.1% HCOOH/MeCN+0.1% HCOOH 60:40 to 40:60) and freeze dried with HCl (0.1N in water) to obtain Example 111 (30 mg, 40%) as a yellow solid.

¹H-NMR (DMSO-d₆, 300 MHz) presence of rotamers δ: 0.98 (s, 1.5H, one rotamer of CH₃); 1.11 (s, 1.5H, other rotamer of CH₃); 1.34-1.36 (m, 3H, CH₃); 1.65-1.95 (m, 4H, 2CH₂); 2.54-2.59 (m, 0.5H, one rotamer of CHₐH_{b}); 2.70-2.78 (m, 0.5H, other rotamer of CHₐH_{b}); 2.95-3.06 (m, 3.5H, CH₃+one rotamer of CHₐH_{b}); 3.24-3.32 (m, 0.5H, other rotamer of CHₐH_{b}); 3.74 (s, 1H, CHₐH_{b}); 3.87 (s, 1H, CHₐH_{b}); 4.05-4.09 (m, 0.5H, one rotamer of CHₐH_{b}); 4.19-4.23 (m, 0.5H, other rotamer of CHₐH_{b}); 4.40-4.52 (m, 1H, CₐH_{b}); 4.57-4.73 (m, 1H, CH); 6.73-6.77 (m, 0.5H, one rotamer of Ar); 6.90-6.93 (m, 0.5H, other rotamer of Ar); 7.12-7.21 (m, 1H, Ar); 7.24-7.26 (m, 1H, Ar); 7.41-7.44 (m, 1H, Ar); 7.50-7.59 (m, 2H, 2Ar); 7.90-8.05 (m, 1H, Ar); 8.50-8.59 (m, 1H, Ar). COOH and HCl were not observed.

M/Z (M[35Cl]+H)⁺: 538.9.

### Compound 141: 2-(3-chloropyridin-2-yl)-2-ethylbutanenitrile

Compound 141 was prepared according to general procedure (I) from 3-chloro-2-fluoropyridine (1.0 g) and cyclopentanecarbonitrile. The crude was purified by flash chromatography (Interchim® 50 μm, CyHex 100% to CyHex/EtOAc 80:20) to obtain Compound 141 (1.28 g, 82%) as a clear oil.

M/Z (M[35Cl]+H⁺): 207.6.

### Compound 142: (1-(3-chloropyridin-2-yl)cyclopentyl)methanamine

Compound 142 (1.10 g, 85%) was obtained as a clear oil following general procedure (XVa), starting from Compound 141 (1.28 g).

M/Z (M[35Cl]+H⁺): 211.6.

### Compound 143: 1′-(3,4-difluorophenyl)-1′,2′-dihydrospiro[cyclopentane-1,3′-pyrrolo[3,2-b]pyridine]

Compound 143 was prepared according to general procedure (Xc) from Compound 142 (400 mg) and 4-bromo-1,2-difluorobenzene. The crude was purified by flash chromatography (Interchim® 50 μm, CyHex 100% to CyHex/EtOAc 85:15) to obtain Compound 143 (181 mg, 33%) as a clear oil.

M/Z (M+H⁺): 287.7.

### Compound 144: 5′-bromo-1′-(3,4-difluorophenyl)-1′,2′-dihydrospiro[cyclopentane-1,3′-pyrrolo[3,2-b]pyridine]

Compound 144 was prepared according to general procedure (XVI) in MeCN from Compound 143 (175 mg). The crude was purified by flash chromatography (Interchim® 50 μm, CyHex 100% to CyHex/EtOAc 80:20) to obtain Compound 144 (150 mg, 67%) as a clear oil.

M/Z (M[⁸¹Br]+H)⁺: 367.6.

### Example 112: methyl 6-(4-(1′-(3,4-difluorophenyl)-1′,2′-dihydrospiro[cyclopentane-1,3′-pyrrolo[3,2-b]pyridine]-5′-carbonyl)-3,3-dimethylpiperazin-1-yl)-2,4-dimethylnicotinate

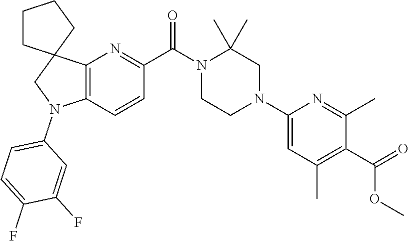 [SMILES: COC(=O)c1c(C)cc(N2CCN(C(=O)c3ccc4c(n3)C3(CCCC3)CN4c3ccc(F)c(F)c3)C(C)(C)C2)nc1C]

Example 112 was prepared according to general procedure (XIV) starting from Compound 144 (100 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to EtOAc 100%) to obtain Example 112 (145 mg) as a white solid.

¹H-NMR (CDCl₃, 300 MHz) δ: 1.80 (s, 6H, (CH₃)₂); 1.68-1.87 (m, 4H, 2CH₂); 1.90-2.23 (m, 4H, 2CH₂); 2.32 (s, 3H, CH₃); 2.48 (s, 3H, CH₃); 3.58-3.66 (m, 2H, N—CH₂); 3.80 (s, 2H, N—CH₂); 4.87 (s, 3H, O—CH₃); 3.93 (s, 2H, N—CH₂); 3.94-3.3.98 (m, 2H, N—CH₂); 6.08 (s, 1H, Ar); 6.88-6.94 (m, 1H, Ar); 6.97-7.04 (m, 1H, Ar); 7.08-7.23 (m, 1H, Ar); 7.26 (d, 1H, J 8.4 Hz, Ar); 7.48 (d, 1H, J 8.4 Hz, Ar).

M/Z (M+H⁺): 591.0.

### Example 113: 6-(4-(1′-(3,4-difluorophenyl)-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid

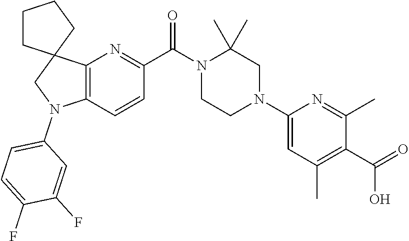 [SMILES: Cc1cc(N2CCN(C(=O)c3ccc4c(n3)C3(CCCC3)CN4c3ccc(F)c(F)c3)C(C)(C)C2)nc(C)c1C(=O)O]

Example 113 was prepared according to general procedure (XI) starting from Example 112 (140 mg). The crude was purified by preparative HPLC (Column B, H₂O+0.1% HCOOH/MeCN+0.1% HCOOH 65:35 to 0:100) and freeze dried with HCl (0.1N in water) to obtain Example 113 (45 mg, 19% over 2 steps) as a white solid.

¹H-NMR (DMSO-d₆, 300 MHz) δ: 1.52 (s, 6H, (CH₃)₂); 1.74-1.91 (m, 8H, (CH₂)₄); 2.34 (s, 3H, CH₃); 3.63-3.72 (m, 2H, CH₂); 3.85-3.91 (m, 4H, (CH₂)₂); 3.94 (s, 2H, CH₂); 6.76-6.90 (m, 1H, Ar); 7.13-7.17 (m, 1H, Ar); 7.34-7.41 (m, 2H, 2Ar); 7.43-7.49 (m, 2H, 2Ar). One CH₃ and COOH were not observed.

M/Z (M+H)⁺: 577.0.

### Compound 145: tert-butyl 4-(4-(2-methoxy-2-oxoethyl)pyridin-2-yl)-2,2-dimethylpiperazine-1-carboxylate

Compound 145 was prepared according to general procedure (Xb), starting from methyl 2-(2-bromopyridin-4-yl)acetate (300 mg) and tert-butyl 2,2-dimethylpiperazine-1-carboxylate (1.2 eq.). The crude was directly engaged in the following step.

M/Z (M+H)⁺: 364.6

### Compound 146: methyl 2-(2-(3,3-dimethylpiperazin-1-yl)pyridin-4-yl)acetate

Compound 146 was obtained as a brown solid (159 mg, 46% over 2 steps) according to general procedure (XII) starting from Compound 145 (261 mg) in DCM using TFA.

M/Z (M+H)⁺: 264.6

### Example 114: methyl 2-(2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-4-yl)acetate

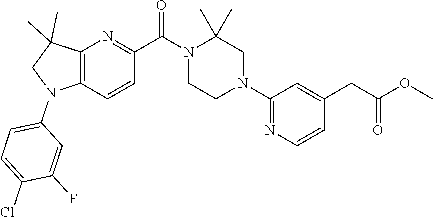 [SMILES: COC(=O)Cc1ccnc(N2CCN(C(=O)c3ccc4c(n3)C(C)(C)CN4c3ccc(Cl)c(F)c3)C(C)(C)C2)c1]

Example 114 was prepared according to general procedure (VIIIb) starting from Compound 68 (60 mg), Compound 146 (1.1 eq.), and N,N-diisopropylethylamine (3.0 eq.) in THF. The crude was purified by flash chromatography (Interchim® 50 μm, DCM 100% to DCM/MeOH 98:02) to obtain Example 114 (70 mg) as a yellow oil.

¹H-NMR (DMSO-d₆, 300 MHz) δ: 1.36 (s, 6H, 2CH₃); 1.50 (s, 6H, 2CH₃); 3.50-3.53 (m, 2H, N—CH₂); 3.60-3.63 (m, 5H, O—CH₃+N—CH₂); 3.81-3.91 (m, 6H, CH₂C(O)⁺2N—CH₂); 6.49-6.54 (m, 2H, 2Ar); 7.17-7.21 (m, 1H, Ar); 7.30-7.37 (m, 2H, 2Ar); 7.52-7.59 (m, 2H, 2Ar); 8.01 (d, J 6.3 Hz, 1H, Ar).

M/Z (M[³⁵Cl]+H)⁺: 567.0.

### Example 115: 2-(2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-4-yl)acetic acid

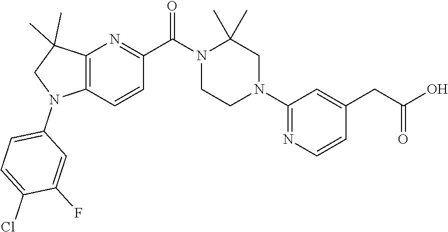 [SMILES: CC1(C)CN(c2ccc(Cl)c(F)c2)c2ccc(C(=O)N3CCN(c4cc(CC(=O)O)ccn4)CC3(C)C)nc21]

Example 115 was prepared according to general procedure (VII) starting from Example 114 (65 mg). The crude was purified by preparative HPLC (Column A, H₂O+0.1% HCOOH/MeCN+0.1% HCOOH 70:30 to 30:70) and freeze dried with HCl (0.1N in water) to obtain Example 115 (45 mg, 68%) as a yellow solid.

¹H-NMR (DMSO-d₆, 300 MHz) δ: 1.37 (s, 6H, 2CH₃); 1.56 (s, 6H, 2CH₃); 3.69-3.73 (m, 2H, CH₂); 3.76 (s, 2H, CH₂); 3.87 (s, 2H, CH₂); 3.94-3.98 (m, 4H, 2CH₂); 6.91 (d, J 6.3 Hz, 1H, Ar); 7.17-7.21 (m, 1H, Ar); 7.33 (dd, J 11.7, 2.6 Hz, 2H, 2Ar); 7.40 (d, J 8.4 Hz, 1H, Ar); 7.53-7.59 (m, 2H, 2Ar); 7.99 (d, J 6.3 Hz, 1H, Ar). COOH was not observed.

M/Z (M[³⁵Cl]+H)⁺: 553.0.

### Compound 147: tert-butyl 4-(6-(2-ethoxy-2-oxoethyl)pyridin-3-yl)-2,2-dimethylpiperazine-1-carboxylate

Compound 147 was prepared according to general procedure (Xb), starting ethyl 2-(5-bromopyridin-2-yl)acetate (300 mg) and tert-butyl 2,2-dimethylpiperazine-1-carboxylate (1.2 eq.). The crude was purified by flash chromatography (Interchim® 50 μm, CyHex 100% to CyHex/EtOAc 65:35) to obtain Compound 147 (273 mg) as a brown solid which was directly engaged in the following step.

M/Z (M+H)⁺: 378.9

### Compound 148: ethyl 2-(5-(3,3-dimethylpiperazin-1-yl)pyridin-2-yl)acetate hydrochloride

Compound 148 was prepared according to general procedure (XII) starting from Compound 147 (273 mg) in DCM using TFA. The crude product was dissolved in DCM (5 mL) and treated with HCl (2M in Et₂O, 3 mL) and concentrated under reduced pressure to obtain Compound 148 as a brown solid (149 mg, 41% over 2 steps)

M/Z (M+H)⁺: 278.5

### Example 116: ethyl 2-(5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-2-yl)acetate

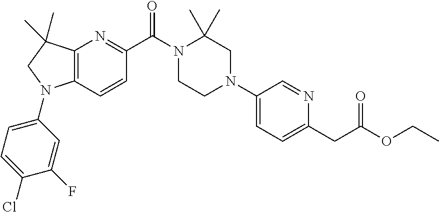 [SMILES: CCOC(=O)Cc1ccc(N2CCN(C(=O)c3ccc4c(n3)C(C)(C)CN4c3ccc(Cl)c(F)c3)C(C)(C)C2)cn1]

Example 116 was prepared according to general procedure (VIIIb) starting from Compound 68 (60 mg), Compound 148 (1.1 eq.), and N,N-diisopropylethylamine (3.0 eq.) in THF. The crude was purified by flash chromatography (Interchim® 50 μm, DCM 100% to DCM/MeOH 98:02) to obtain Example 116 (60 mg, 55%) as a yellow oil.

¹H-NMR (DMSO-d₆, 300 MHz) δ: 1.17 (t, 3H, J 7.2 Hz, CH₂CH₃); 1.35 (s, 6H, 2CH₃); 1.52 (s, 6H, 2CH₃); 3.33-3.44 (m, 2H, N—CH₂); 3.54 (s, 2H, CH₂C(O)); 3.66 (s, 2H, N—CH₂); 3.79-3.86 (m, 4H, 2N—CH₂); 4.05 (q, 2H, J 7.2 Hz, CH₂CH₃); 7.14-7.21 (m, 3H, 3Ar); 7.30-7.37 (m, 2H, 2Ar); 7.53-7.59 (m, 2H, 2Ar); 8.05 (s, 1H, Ar). COOH was not observed.

M/Z (M[³⁵Cl]+H)⁺: 581.0.

### Example 117: 2-(5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-2-yl)acetic acid

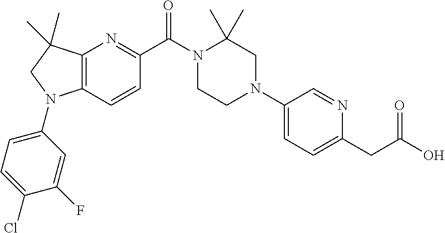 [SMILES: CC1(C)CN(c2ccc(Cl)c(F)c2)c2ccc(C(=O)N3CCN(c4ccc(CC(=O)O)nc4)CC3(C)C)nc21]

Example 117 was prepared according to general procedure (VII) starting from Example 116 (60 mg). The crude was purified by preparative HPLC (Column A, H₂O+0.1% HCOOH/MeCN+0.1% HCOOH 70:30 to 30:70) and freeze dried with HCl (0.1N in water) to obtain Example 117 (42 mg, 69%) as a yellow solid.

¹H-NMR (DMSO-d₆, 300 MHz) δ: 1.36 (s, 6H, 2CH₃); 1.54 (s, 6H, 2CH₃); 3.51-3.54 (m, 2H, CH₂); 3.74 (s, 2H, CH₂); 3.87-3.93 (m, 4H, 2CH₂); 4.00 (s, 2H, CH₂); 7.17-7.21 (m, 1H, Ar); 7.33 (dd, J 11.9, 2.5 Hz, 1H, Ar); 7.38 (d, J 8.4 Hz, 1H, Ar); 7.53-7.59 (m, 2H, 2Ar); 7.74 (d, J 9.0 Hz, 1H, Ar); 7.88-7.92 (m, 1H, Ar); 8.23-8.24 (m, 1H, Ar). COOH was not observed.

M/Z (M[³⁵Cl]+H)⁺: 552.9.

### Compound 149: 1-(3-chloropyridin-2-yl)cyclobutane-1-carbonitrile

Compound 149 was prepared according to general procedure (I) from 3-chloro-2-fluoropyridine (1.0 g) and cyclobutanecarbonitrile. The crude was purified by flash chromatography (Interchim® 50 μm, CyHex 100% to CyHex/EtOAc 80:20) to obtain Compound 149 (0.76 g, 52%) as a clear oil.

M/Z (M[³⁵Cl]+H⁺): 193.6

### Compound 150: (1-(3-chloropyridin-2-yl)cyclobutyl)methanamine

Compound 150 (293 mg, 38%) was obtained as a yellow oil following general procedure (XVa), starting from Compound 149 (756 mg).

M/Z (M[³⁵Cl]+H⁺): 197.6.

### Compound 151: 1′-(4-chloro-3-fluorophenyl)-1′,2′-dihydrospiro[cyclobutane-1,3′-pyrrolo[3,2-b]pyridine]

Compound 150 was prepared according to general procedure (Xc) from Compound 149 (293 mg) and 4-bromo-1-chloro-2-fluorobenzene. The crude was purified by flash chromatography (Interchim® 50 μm, CyHex 100% to CyHex/EtOAc 80:20) to obtain Compound 151 (187 mg, 43%) as a yellow oil.

M/Z (M[³⁵Cl]+H⁺): 287.7.

### Compound 152: 5′-bromo-1′-(4-chloro-3-fluorophenyl)-1′,2′-dihydrospiro[cyclobutane-1,3′-pyrrolo[3,2-b]pyridine]

Compound 152 was prepared according to general procedure (XVI) in MeCN from Compound 151 (187 mg). The crude was purified by flash chromatography (Interchim® 50 μm, CyHex 100% to CyHex/EtOAc 90:10) to obtain Compound 152 (151 mg, 63%) as a yellow oil.

M/Z (M[³⁵Cl][⁸¹Br]+H)⁺: 369.6.

### Example 118: methyl 2-(6-(4-(1′-(4-chloro-3-fluorophenyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetate

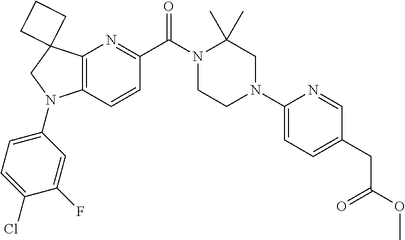 [SMILES: COC(=O)Cc1ccc(N2CCN(C(=O)c3ccc4c(n3)C3(CCC3)CN4c3ccc(Cl)c(F)c3)C(C)(C)C2)nc1]

Example 118 was prepared according to general procedure (XIV) starting from Compound 152 (70 mg) and Compound 111 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 60:40) to obtain Example 118 (51 mg) as a white solid.

M/Z (M[³⁵Cl]+H)⁺: 579.0

### Example 119: 2-(6-(4-(1′-(4-chloro-3-fluorophenyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid

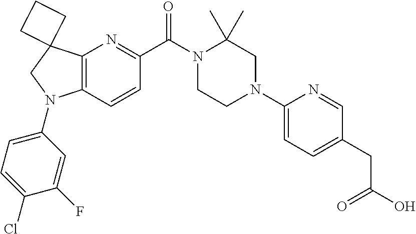 [SMILES: CC1(C)CN(c2ccc(CC(=O)O)cn2)CCN1C(=O)c1ccc2c(n1)C1(CCC1)CN2c1ccc(Cl)c(F)c1]

Example 119 was prepared according to general procedure (VII) starting from Example 118 (50 mg). The crude was purified by preparative HPLC (Column B, H₂O+0.1% HCOOH/MeCN+0.1% HCOOH 95:05 to 0:100) and freeze dried with HCl (0.1N in water) to obtain Example 119 (28 mg, 24% over 2 steps) as a yellow solid.

¹H-NMR (DMSO-d₆+D₂O, 300 MHz) δ: 1.53 (s, 6H, (CH₃)₂); 1.98-2.08 (m, 3H, CH+CH₂); 2.19-2.23 (m, 3H, CH+CH₂); 2.41-2.44 (m, 2H, CH₂C(O)); 3.56 (s, 2H, N—CH₂); 3.63-3.66 (m, 2H, N—CH₂); 3.93-3.99 (m, 2H, N—CH₂); 4.14 (bs, 2H, N—CH₂); 7.14-7.18 (m, 2H, 2Ar); 7.24-7.27 (m, 1H, Ar); 7.34-7.37 (m, 1H, Ar); 7.48-7.53 (m, 2H, 2Ar); 7.92-7.95 (m, 2H, 2Ar). COOH was not observed.

M/Z (M[³⁵Cl]+H)⁺: 564.9

### Compound 153: Spiro[indoline-3,3′-pyrrolidin]-2-one hydrochloride

Compound 153 was prepared according to general procedure (XII) starting from tert-butyl 2-oxospiro[indoline-3,3′-pyrrolidine]-1′-carboxylate (100 mg) in DCM using TFA. The crude product was dissolved in DCM (5 mL) and treated with HCl (2M in Et₂O, 3 mL) and concentrated under reduced pressure to obtain Compound 153 as a yellow solid (77 mg, 99%)

M/Z (M+H)+: not observed

### Example 120: 1′-(1-(4-chloro-3-fluorophenyl)-3,3-dimethyl-2,3-dihydro-1H-pyrrolo[3,2-b]pyridine-5-carbonyl)spiro[indoline-3,3′-pyrrolidin]-2-one

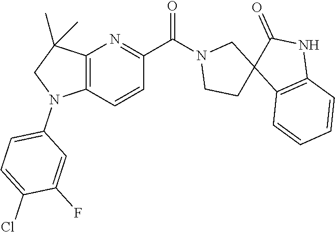 [SMILES: CC1(C)CN(c2ccc(Cl)c(F)c2)c2ccc(C(=O)N3CCC4(C3)C(=O)Nc3ccccc34)nc21]

Example 120 was prepared according to general procedure (VIIIb) starting from Compound 68 (60 mg), Compound 153 (1.1 eq.), and N,N-diisopropylethylamine (3.0 eq.) in MeTHF. The crude was purified by flash chromatography (Interchim® 50 μm, DCM 100% to DCM/MeOH 98:02) then further purified by preparative HPLC (Column A, H₂O+0.1% HCOOH/MeCN+0.1% HCOOH 50:50 to 10:90) and freeze dried with HCl (0.1N in water) to obtain Example 120 (63 mg, 64%) as a yellow solid.

¹H-NMR (DMSO-d₆, 300 MHz) δ: 1.24-1.25 (m, 3H, CH₃); 1.38-1.39 (m, 3H, CH₃); 2.11-2.34 (m, 2H, CH₂); 3.74-3.84 (m, 2H, N—CH₂); 3.90-3.95 (m, 2H, N—CH₂); 3.99-4.07 (m, 1H, N—CHₐH_{b}); 4.21-4.36 (m, 1H, N—CHₐH_{b}); 6.84-6.90 (m, 1H, Ar); 6.95-7.03 (m, 1H, Ar); 7.15-7.39 (m, 4H, 4Ar); 7.51-7.59 (m, 3H, 3Ar); 10.51-10.56 (m, 1H, NH).

M/Z (M[³⁵Cl]+H)⁺: 491.8.

### Compound 154: 1-oxa-3,8-diazaspiro[4.5]decan-2-one hydrochloride

Compound 154 was prepared according to general procedure (XII) starting tert-butyl 2-oxo-1-oxa-3,8-diazaspiro[4.5]decane-8-carboxylate (100 mg) in DCM using TFA. The crude product was dissolved in DCM (5 mL) and treated with HCl (2M in Et₂O, 3 mL) and concentrated under reduced pressure to obtain Compound 154 as a yellow oil (74 mg, 99%)

M/Z (M+H)+: not observed

### Example 121: 8-(1-(4-chloro-3-fluorophenyl)-3,3-dimethyl-2,3-dihydro-1H-pyrrolo[3,2-b]pyridine-5-carbonyl)-1-oxa-3,8-diazaspiro[4.5]decan-2-one

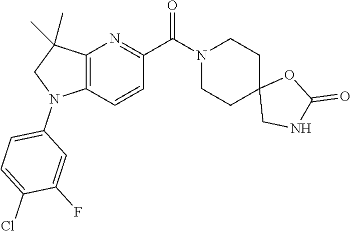 [SMILES: CC1(C)CN(c2ccc(Cl)c(F)c2)c2ccc(C(=O)N3CCC4(CC3)CNC(=O)O4)nc21]

Example 121 was prepared according to general procedure (VIIIb) starting from Compound 68 (60 mg), Compound 154 (1.1 eq.), and N,N-diisopropylethylamine (3.0 eq.) in MeTHF. The crude was purified by preparative HPLC (Column A, H₂O+0.1% HCOOH/MeCN+0.1% HCOOH 60:40 to 20:80) and freeze dried with HCl (0.1N in water) to obtain Example 121 (20 mg, 22%) as a yellow solid.

¹H-NMR (DMSO-d₆, 300 MHz) δ: 1.33 (s, 6H, 2CH₃); 1.77-1.90 (m, 4H, 2CH₂); 3.31 (s, 2H, CH₂); 3.45-3.56 (m, 2H, CH₂); 3.75-4.00 (m, 4H, 2CH₂); 7.17-7.20 (m, 1H, Ar); 7.31-7.36 (m, 1H, Ar); 7.39-7.42 (m, 1H, Ar); 7.52-7.59 (m, 3H, Ar+NH).

M/Z (M[³⁵Cl]+H)⁺: 459.7.

### Compound 155: tert-butyl 6-(5-(ethoxycarbonyl)pyridin-2-yl)-2,6-diazaspiro[3.3]heptane-2-carboxylate

Compound 155 was prepared according to general procedure (Xb), starting from ethyl 6-chloronicotinate (200 mg) and tert-butyl 2,6-diazaspiro[3.3]heptane-2-carboxylate (1.2 eq.). The crude was purified by flash chromatography (Interchim® 50 μm, CyHex 100% to CyHex/EtOAc 60:40) to obtain Compound 155 (242 mg, 65%) as a yellow oil.

M/Z (M+H)⁺: 348.8

### Compound 156: ethyl 6-(2,6-diazaspiro[3.3]heptan-2-yl)nicotinate

Compound 156 was prepared according to general procedure (XII) starting from Compound 155 (242 mg) in DCM using TFA. The reaction mixture was concentrated under reduced pressure to obtain the crude Compound 156 (135 mg) as a brown solid.

M/Z (M+H)⁺: 248.7

### Example 122: ethyl 6-(6-(1-(4-chloro-3-fluorophenyl)-3,3-dimethyl-2,3-dihydro-1H-pyrrolo[3,2-b]pyridine-5-carbonyl)-2,6-diazaspiro[3.3]heptan-2-yl)nicotinate

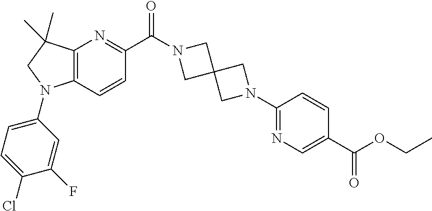 [SMILES: CCOC(=O)c1ccc(N2CC3(CN(C(=O)c4ccc5c(n4)C(C)(C)CN5c4ccc(Cl)c(F)c4)C3)C2)nc1]

Example 122 was prepared according to general procedure (VIIIb) starting from Compound 68 (60 mg), Compound 156 (1.1 eq.), and N,N-diisopropylethylamine (3.0 eq.) in THF. The crude was purified by flash chromatography (Interchim® 50 μm, CyHex 100% to CyHex/EtOAc 50:50) to obtain Example 122 (85 mg, 83%) as a yellow solid.

¹H-NMR (DMSO-d₆, 300 MHz) δ: 1.28 (t, J 7.2 Hz, 3H, CH₂CH₃); 1.36 (s, 6H, 2CH₃); 3.88 (s, 2H, N—CH₂); 4.21-4.30 (m, 8H, CH₂CH₃+3N—CH₂); 4.86 (s, 2H, N—CH₂); 6.37 (d, J 8.8 Hz, 1H, Ar); 7.18-7.22 (m, 1H, Ar); 7.32-7.37 (m, 1H, Ar); 7.53-7.59 (m, 2H, 2Ar); 7.72 (d, J 8.4 Hz, 1H, Ar); 7.92-7.96 (m, 1H, Ar); 8.61 (m, 1H, Ar).

M/Z (M[³⁵Cl]+H)⁺: 551.0.

### Example 123: 6-(6-(1-(4-chloro-3-fluorophenyl)-3,3-dimethyl-2,3-dihydro-1H-pyrrolo[3,2-b]pyridine-5-carbonyl)-2,6-diazaspiro[3.3]heptan-2-yl)nicotinic acid

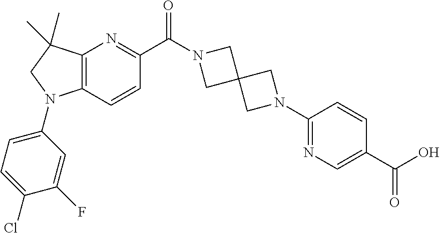 [SMILES: CC1(C)CN(c2ccc(Cl)c(F)c2)c2ccc(C(=O)N3CC4(C3)CN(c3ccc(C(=O)O)cn3)C4)nc21]

Example 123 was prepared according to general procedure (VII) starting from Example 122 (60 mg). The crude was purified by preparative HPLC (Column A, H₂O+0.1% HCOOH/MeCN+0.1% HCOOH 70:30 to 30:70) and freeze dried with HCl (0.1N in water) to obtain Example 123 (42 mg, 69%) as a yellow solid.

¹H-NMR (DMSO-d₆, 300 MHz) δ: 1.36 (s, 6H, 2CH₃); 3.88 (s, 2H, CH₂); 4.20-4.28 (m, 6H, 3CH₂); 4.86 (s, 2H, CH₂); 6.37 (d, J 8.8 Hz, 1H, Ar); 7.18-7.22 (m, 1H, Ar); 7.32-7.37 (m, 1H, Ar); 7.53-7.59 (m, 2H, 2 Ar); 7.72 (d, J 8.4 Hz, 1H, Ar); 7.90-7.94 (m, 1H, Ar); 8.59 (m, 1H, Ar). COOH was not observed.

M/Z (M[³⁵Cl]+H)⁺: 522.8.

### Compound 157: methyl 1′-(4-chloro-3-fluorophenyl)-1′,2′-dihydrospiro[cyclobutane-1,3′-pyrrolo[3,2-b]pyridine]-5′-carboxylate

Compound 157 was prepared according to general procedure (XIX) starting from Compound 152 (230 mg). The crude Compound 157 (217 mg) was obtained as a red solid and was directly engaged in the following step.

M/Z (M[³⁵Cl]+H)⁺: 347.7

### Compound 158: 1′-(4-chloro-3-fluorophenyl)-1′,2′-dihydrospiro[cyclobutane-1,3′-pyrrolo[3,2-b]pyridine]-5′-carboxylic acid

Compound 158 was prepared according to general procedure (VII) starting from Compound 157 (217 mg). The crude Compound 158 (160 mg, 72%) was obtained as an orange solid.

M/Z (M[³⁵Cl]+H)⁺: 333.6

### Example 124: methyl 2-(1-(1′-(4-chloro-3-fluorophenyl)-1′,2′-dihydrospiro[cyclobutane-1,3′-pyrrolo[3,2-b]pyridine]-5′-carbonyl)piperidin-4-yl)acetate

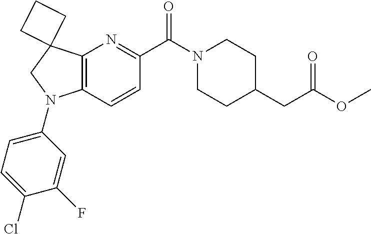 [SMILES: COC(=O)CC1CCN(C(=O)c2ccc3c(n2)C2(CCC2)CN3c2ccc(Cl)c(F)c2)CC1]

Example 124 was prepared according to general procedure (VIIIb) starting from Compound 158 (100 mg), methyl 2-(piperidin-4-yl)acetate (1.1 eq.), and N,N-diisopropylethylamine (3.0 eq.) in MeTHF. The crude was purified by flash chromatography (Interchim® 50 μm, CyHex 100% to EtOAc 100%) to obtain Example 124 (69 mg, 49%) as a clear oil.

M/Z (M[³⁵Cl]+H)⁺: 472.7

### Example 125: 2-(1-(1′-(4-chloro-3-fluorophenyl)-1′,2′-dihydrospiro[cyclobutane-1,3′-pyrrolo[3,2-b]pyridine]-5′-carbonyl)piperidin-4-yl)acetic acid

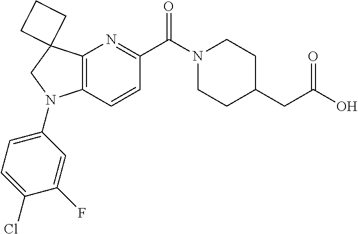 [SMILES: O=C(O)CC1CCN(C(=O)c2ccc3c(n2)C2(CCC2)CN3c2ccc(Cl)c(F)c2)CC1]

Example 125 was prepared according to general procedure (VII) starting from Example 124 (65 mg). The crude was suspended in Et₂O (5 mL), the solid was isolated by filtration then freeze dried in HCl (0.1N in water) to obtain Example 125 (40 mg, 59%) as a yellow solid.

¹H-NMR (DMSO-d₆, 300 MHz) δ: 1.15-1.33 (m, 2H, CH₂); 1.66-1.82 (m, 2H, CH₂); 1.90-2.02 (m, 2H, CH₂); 2.04-2.15 (m, 1H, CH); 2.19-2.21 (m, 2H, CH₂); 2.24-2.31 (m, 2H, CH₂); 2.36-2.43 (m, 2H, CH₂); 2.74-2.84 (m, 1H, CH); 3.03-3.12 (m, 1H, CH); 4.11-4.17 (m, 3H, CH₂, CH); 4.42-4.47 (m, 1H, CH); 7.17 (dd, J 8.7, 2.1 Hz, 1H, Ar); 7.30 (dd, J 11.7, 2.1 Hz, 1H, Ar); 7.35 (d, J 8.7 Hz, 1H, Ar); 7.50-7.56 (m, 2H, 2Ar); 12.10 (bs, 1H, CO₂H).

M/Z (M[³⁵Cl]+H)⁺: 458.8.

### Example 126: 4-(1′-(4-chloro-3-fluorophenyl)-1′,2′-dihydrospiro[cyclobutane-1,3′-pyrrolo[3,2-b]pyridine]-5′-carbonyl)-3,3-dimethylpiperazin-2-one

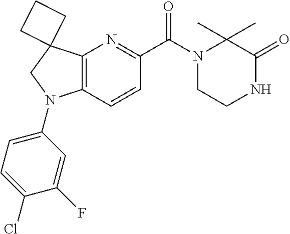 [SMILES: CC1(C)C(=O)NCCN1C(=O)c1ccc2c(n1)C1(CCC1)CN2c1ccc(Cl)c(F)c1]

Example 126 was prepared according to general procedure (VIIIb) starting from Compound 158 (50 mg), 3,3-dimethylpiperazin-2-one (1.1 eq.), and N,N-diisopropylethylamine (3.0 eq.) in MeTHF. The crude was purified by preparative HPLC (Column B, H₂O+0.1% HCOOH/MeCN+0.1% HCOOH 65:35 to 0:100) and freeze dried with water to obtain Example 126 (15 mg, 22%) as a white solid.

¹H-NMR (DMSO-d₆, 300 MHz) δ: 1.67 (s, 6H, (CH₃)₂); 1.97-2.10 (m, 2H, CH₂); 2.25-2.31 (m, 2H, CH₂); 2.30-2.35 (m, 2H, CH₂); 3.34-3.39 (m, 2H, CH₂); 3.56-3.60 (m, 2H, CH₂); 4.18 (s, 2H, CH₂); 7.17 (dd, J 8.7, 2.4 Hz, 1H, Ar); 7.31 (dd, J 11.7, 2.4 Hz, 1H, Ar); 7.41 (d, J 8.7 Hz, 1H, Ar); 7.51-7.57 (m, 2H, 2Ar), 8.08 (bs, 1H, NH).

M/Z (M[³⁵Cl]+H)⁺: 443.7

### Example 127: methyl 2-(6-(4-(1′-(4-chloro-3-fluorophenyl)-3,3-difluoro-1′,2′-dihydrospiro[cyclobutane-1,3′-pyrrolo[3,2-b]pyridine]-5′-carbonyl)-3,3-dimethylpiperazin-1-yl)pyridin-3-yl)acetate

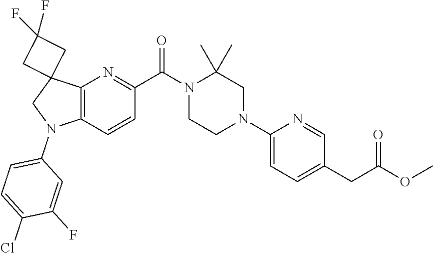 [SMILES: COC(=O)Cc1ccc(N2CCN(C(=O)c3ccc4c(n3)C3(CN4c4ccc(Cl)c(F)c4)CC(F)(F)C3)C(C)(C)C2)nc1]

Example 127 was prepared according to general procedure (XIV) starting from Compound 125 (70 mg) and Compound 111 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 60:40) to obtain Example 127 (106 mg, 51%) as a white solid.

¹H-NMR (DMSO-d₆, 300 MHz) δ: 1.52 (s, 6H, (CH₃)₂); 2.92-3.14 (m, 4H, (CH₂)₂); 3.45-3.58 (m, 4H, 2N—CH₂); 3.61 (s, 2H, CH₂C(O)); 3.80-3.87 (m, 5H, N—CH₂+OCH₃); 4.32 (s, 2H, N—CH₂); 6.54 (d, J 8.7 Hz, 1H, Ar); 7.19-7.21 (m, 1H, Ar); 7.33-7.45 (m, 3H, 3Ar); 7.53-7.60 (m, 2H, 2Ar); 7.90-7.94 (m, 1H, Ar).

M/Z (M[³⁵Cl]+H)⁺: 614.9

### Example 128: 2-(6-(4-(1′-(4-chloro-3-fluorophenyl)-3,3-difluoro-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid

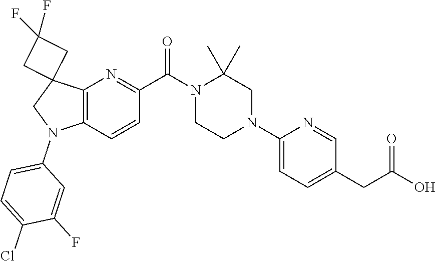 [SMILES: CC1(C)CN(c2ccc(CC(=O)O)cn2)CCN1C(=O)c1ccc2c(n1)C1(CN2c2ccc(Cl)c(F)c2)CC(F)(F)C1]

Example 128 was prepared according to general procedure (VII) starting from Example 127 (50 mg). The crude was purified by preparative HPLC (Column B, H₂O+0.1% HCOOH/MeCN+0.1% HCOOH 95:05 to 0:100) and freeze dried with HCl (0.1N in water) to obtain Example 128 (40 mg, 39%) as a yellow solid.

¹H-NMR (DMSO-d₆, 300 MHz) δ: 1.56 (s, 6H, (CH₃)₂); 2.92-3.17 (m, 4H, (CH₂)₂); 3.60-3.65 (m, 2H, CH₂C(O)); 3.65-3.69 (m, 2H, N—CH₂); 3.75-3.91 (m, 4H, 2N—CH₂); 4.33 (s, 2H, N—CH₂); 7.19-7.29 (m, 2H, 2Ar); 7.34-7.37 (m, 1H, Ar); 7.43-7.46 (m, 1H, Ar); 7.54-7.61 (m, 2H, 2Ar); 7.90-7.94 (m, 2H, 2Ar). COOH was not observed

M/Z (M[³⁵Cl]+H)⁺: 600.4

### Compound 159: benzyl (1R,5S)-3-(2-ethoxy-2-oxoethylidene)-8-azabicyclo[3.2.1]octane-8-carboxylate

To a solution of Sodium Hydride (60% in mineral oil, 154 mg) in THE (2 mL) at 0° C. was added dropwise a solution of ethyl 2-(diethoxyphosphoryl)acetate (865 mg) in THE (5 mL) the reaction mixture was stirred at 0° C. for 10 minutes then a solution of Benzyl (1S,5R)-3-oxo-8-azabicyclo[3.2.1]octane-8-carboxylate (500 mg) in THE (5 mL) was added and the reaction mixture was stirred at 25° C. for 5 hours. The reaction mixture was quenched with water (50 mL) then extracted with EtOAc (50 mL). The organic layer was washed with brine (50 mL), dried over MgSO₄ then concentrated under reduced pressure. The crude was purified by flash chromatography (Interchim® 50 μm, CyHex 100% to CyHex/EtOAc 80:20) to obtain Compound 159 (488 mg, 77%) as a clear oil.

M/Z (M+H)⁺: 330.8.

### Compound 160: ethyl 2-((1R,5S)-8-azabicyclo[3.2.1]octan-3-yl)acetate

A solution of of Compound 159 (488 mg) in EtOH (4 mL) was sparged with Argon for 5 minutes then Palladium on Charcoal (10% w/w, 0.1 eq) was added. The reaction mixture was stirred under a hydrogen atmosphere (1 Bar) for 16 hours, then filtered over a Celite pad which was rinsed with EtOAc (50 mL). The filtrate was concentrated under reduced pressure. The crude was purified by flash chromatography (Interchim® 50 μm, DCM 100% to DCM/MeOH 80:20) to obtain Compound 160 (190 mg, 65%) as a yellow oil.

M/Z (M+H)⁺: 198.7.

### Example 129: ethyl 2-((1R,5S)-8-(1-(4-chloro-3-fluorophenyl)-3,3-dimethyl-2,3-dihydro-1H-pyrrolo[3,2-b]pyridine-5-carbonyl)-8-azabicyclo[3.2.1]octan-3-yl)acetate

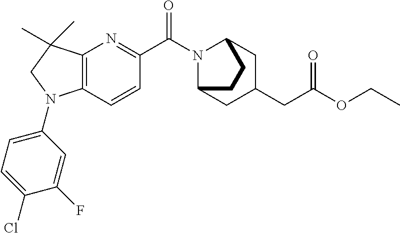

Example 129 was prepared according to general procedure (VIIIb) starting from Compound 68 (90 mg), and Compound 160 (1.1 eq.), and N,N-diisopropylethylamine (3.0 eq.) in MeTHF. The crude Example 129 (180 mg) was obtained as yellow oil and directly engaged in the following step.

M/Z (M[³⁵Cl]+H)⁺: 500.9

### Example 130: 2-((1R,5S)-8-(1-(4-chloro-3-fluorophenyl)-3,3-dimethyl-2,3-dihydro-1H-pyrrolo[3,2-b]pyridine-5-carbonyl)-8-azabicyclo[3.2.1]octan-3-yl)acetic acid

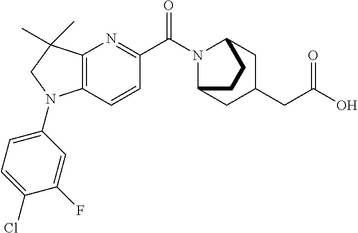

Example 130 was prepared according to general procedure (VII) starting from Example 129 (141 mg). The crude was purified by preparative HPLC (Column B, H₂O+0.1% HCOOH/MeCN+0.1% HCOOH 65:35 to 0:100) and freeze dried with HCl (0.1N in water) to obtain the diasteroisomere mixture (85:15) Example 130 (74 mg, 56% over 2 steps) as a white solid.

¹H-NMR (MeOD-d₄, 300 MHz) δ: 1.42 (s, 6H, CH₃×2); 1.48-1.57 (m, 1.5H); 1.72-1.82 (m, 0.5H); 1.83-2.12 (m, 4H); 2.13-2.52 (m, 4H); 2.52-2.67 (m, 1H); 3.86 (s, 2H, CH₂); 4.69-4.86 (m, 2H); 7.11-7.19 (m, 1H, Ar); 7.20 (dd, J 11.7, 2.5 Hz, 1H, Ar); 7.46 (t, J 8.7, 1H, Ar); 7.48-7.62 (m, 2H, 2Ar); COOH was not observed.

M/Z (M[³⁵Cl]+H)⁺: 472.3

### Compound 161: 5-chloro-1-(5-chloropyridin-2-yl)-3,3-dimethyl-2,3-dihydro-1H-pyrrolo[3,2-b]pyridine

Compound 161 was prepared according to general procedure (X) starting from Compound 70 (350 mg) and 2-bromo-5-chloropyridine (2.0 eq) with ^{t}BuONa (3.0 eq) at 100° C. for 18 hours. The crude was purified by flash chromatography (Interchim® 50 μm, CyHex 100% to CyHex/DCM 0:100) to obtain Compound 161 (150 mg, 62%) as a beige solid.

M/Z (M[³⁵Cl]+H)⁺: 294.6.

### Example 131: methyl 6-(4-(6-(5-(4-(5-(methoxycarbonyl)-4,6-dimethylpyridin-2-yl)-2,2-dimethylpiperazine-1-carbonyl)-3,3-dimethyl-2,3-dihydro-1H-pyrrolo[3,2-b]pyridin-1-yl)nicotinoyl)-3,3-dimethylpiperazin-1-yl)-2,4-dimethylnicotinate

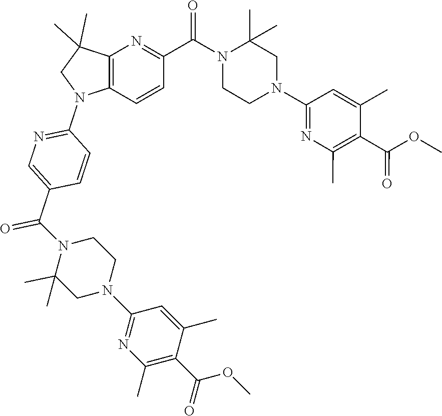 [SMILES: COC(=O)c1c(C)cc(N2CCN(C(=O)c3ccc(N4CC(C)(C)c5nc(C(=O)N6CCN(c7cc(C)c(C(=O)OC)c(C)n7)CC6(C)C)ccc54)nc3)C(C)(C)C2)nc1C]

### And Example 132: methyl 6-(4-(1-(5-chloropyridin-2-yl)-3,3-dimethyl-2,3-dihydro-1H-pyrrolo[3,2-b]pyridine-5-carbonyl)-3,3-dimethylpiperazin-1-yl)-2,4-dimethylnicotinate

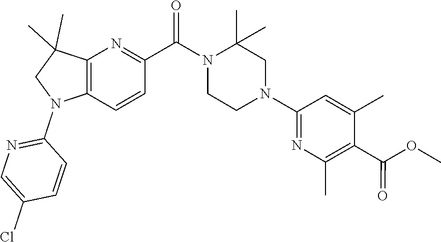 [SMILES: COC(=O)c1c(C)cc(N2CCN(C(=O)c3ccc4c(n3)C(C)(C)CN4c3ccc(Cl)cn3)C(C)(C)C2)nc1C]

Example 131 and Example 132 were prepared according to general procedure (XIV) starting from Compound 161 (75 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 20:80) to obtain Example 131 (76 mg, 36%) as a white solid and Example 132 (40 mg, 28%) as a white solid.

### Example 131

¹H-NMR (DMSO-d₆, 300 MHz) δ: 1.39 (s, 6H, 2CH₃); 1.49 (s, 12H, 4CH₃); 2.23 (s, 6H, 2CH₃); 2.33 (s, 6H, 2CH₃); 3.47-3.59 (m, 6H, 3N—CH₂); 3.76-3.83 (m, 4H, 2N—CH₂); 3.87-3.92 (m, 4H, 2N—CH₂); 3.98 (s, 2H, N—CH₂); 6.36 (s, 2H, 2Ar); 6.97 (d, J 8.9 Hz, 1H, Ar); 7.43 (d, J 8.4 Hz, 1H, Ar); 7.85 (dd, J 8.9, 2.2 Hz, 1H, Ar); 8.43 (d, J 2.2 Hz, 1H, Ar); 8.59 (d, J 8.4 Hz, 1H, Ar);

M/Z (M+H)⁺: 833.7

### Example 132

¹H-NMR (DMSO-d₆, 300 MHz) δ: 1.39 (s, 6H, 2CH₃); 1.50 (s, 6H, 2CH₃); 2.24 (s, 3H, CH₃); 2.35 (s, 3H, CH₃); 3.53- 3.60 (m, 2H, N—CH₂); 3.76-3.84 (m, 5H, O—CH₃+N—CH₂); 3.89 (s, 2H, N—CH₂); 3.93 (s, 2H, N—CH₂); 6.37 (s, 1H, Ar); 7.00 (d, J 9.1 Hz, 1H, Ar); 7.42 (d, J 8.5 Hz, 1H, Ar); 7.88 (dd, J 9.1, 2.5 Hz, 1H, Ar); 8.39 (d, J 2.5 Hz, 1H, Ar); 8.47 (d, J 8.5 Hz, 1H, Ar);

M/Z (M[³⁵Cl]+H)⁺: 549.3.

### Example 133: 6-(4-(6-(5-(4-(5-carboxy-4,6-dimethylpyridin-2-yl)-2,2-dimethylpiperazine-1-carbonyl)-3,3-dimethyl-2,3-dihydro-1H-pyrrolo[3,2-b]pyridin-1-yl)nicotinoyl)-3,3-dimethylpiperazin-1-yl)-2,4-dimethylnicotinic acid

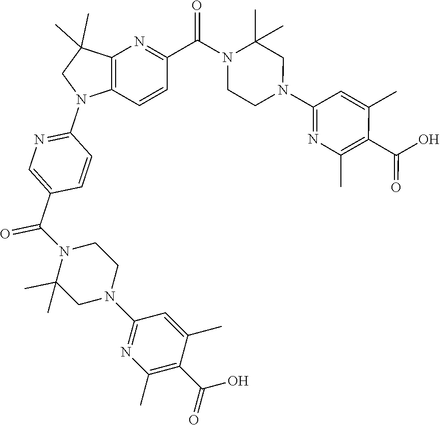 [SMILES: Cc1cc(N2CCN(C(=O)c3ccc(N4CC(C)(C)c5nc(C(=O)N6CCN(c7cc(C)c(C(=O)O)c(C)n7)CC6(C)C)ccc54)nc3)C(C)(C)C2)nc(C)c1C(=O)O]

Example 133 was prepared according to general procedure (XI) starting from Example 131 (76 mg). The crude was purified by preparative HPLC (Column A, H₂O+0.1% HCOOH/MeCN+0.1% HCOOH 90:10 to 50:50) and freeze dried with HCl (0.1N in water) to obtain Example 133 (3 mg, 4%) as a white solid.

¹H-NMR (DMSO-d₆, 300 MHz) δ: 1.40 (s, 6H, 2CH₃); 1.50 (s, 12H, 4CH₃); 2.26 (s, 6H, 2CH₃); 2.37 (s, 6H, 2CH₃); 3.49-3.58 (m, 4H, 2N—CH₂); 3.76-3.83 (m, 4H, 2N—CH₂); 3.87-3.89 (m, 4H, 2N—CH₂); 3.97 (s, 2H, N—CH₂); 6.34 (s, 2H, 2Ar); 6.99 (d, J 8.9 Hz, 1H, Ar); 7.44 (d, J 8.4 Hz, 1H, Ar); 7.86 (dd, J 8.9, 2.2 Hz, 1H, Ar); 8.45 (d, J 2.2 Hz, 1H, Ar); 8.6 (d, J 8.4 Hz, 1H, Ar). 2COOH signals were not observed.

M/Z (M+2H)/2⁺: 403.0.

### Example 134: 6-(4-(1-(5-chloropyridin-2-yl)-3,3-dimethyl-2,3-dihydro-1H-pyrrolo[3,2-b]pyridine-5-carbonyl)-3,3-dimethylpiperazin-1-yl)-2,4-dimethylnicotinic acid

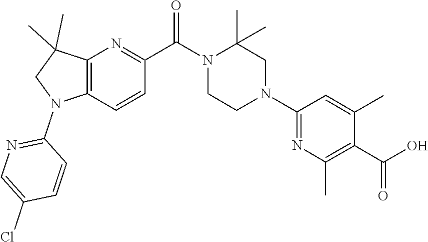 [SMILES: Cc1cc(N2CCN(C(=O)c3ccc4c(n3)C(C)(C)CN4c3ccc(Cl)cn3)C(C)(C)C2)nc(C)c1C(=O)O]

Example 134 was prepared according to general procedure (XI) starting from Example 132 (70 mg). The crude was purified by preparative HPLC (Column A, H₂O+0.1% HCOOH/MeCN+0.1% HCOOH 70:30 to 30:70) and freeze dried with HCl (0.1N in water) to obtain Example 133 (25 mg, 36%) as a white solid.

¹H-NMR (DMSO-d₆, 300 MHz) δ: 1.38 (s, 6H, 2CH₃); 1.50 (s, 6H, 2CH₃); 2.26 (s, 3H, CH₃); 2.37 (s, 3H, CH₃); 3.53- 3.56 (m, 2H, N—CH₂); 3.79-3.82 (m, 2H, N—CH₂); 3.89 (s, 2H, N—CH₂); 3.92 (s, 2H, N—CH₂); 6.34 (s, 1H, Ar); 7.00 (d, J 9.1 Hz, 1H, Ar); 7.42 (d, J 8.5 Hz, 1H, Ar); 7.88 (dd, J 9.1, 2.5 Hz, 1H, Ar); 8.39 (d, J 2.5 Hz, 1H, Ar); 8.47 (d, J 8.5 Hz, 1H, Ar). COOH was not observed.

M/Z (M[³⁵Cl]+H)⁺: 549.3.

### Example 135: methyl 6-(4-(3,3-dimethyl-1-(pyridin-2-yl)-2,3-dihydro-1H-pyrrolo[3,2-b]pyridine-5-carbonyl)-3,3-dimethylpiperazin-1-yl)-2,4-dimethylnicotinate

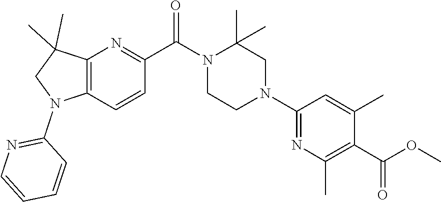 [SMILES: COC(=O)c1c(C)cc(N2CCN(C(=O)c3ccc4c(n3)C(C)(C)CN4c3ccccn3)C(C)(C)C2)nc1C]

To a solution of Example 132 (20 mg) in dioxane (1 mL), was added potassium trifluoro(vinyl)borate (6 mg) and potassium carbonate (1.2 M in water, 89 μL). The reaction mixture was sparged with Argon for 10 minutes then Bis(tri-tert-butylphosphine)palladium (2 mg) was added and the reaction mixture was heated at 130° C. for 96 hours, then filtered over celite, and concentrated under reduced pressure. The residue was purified by preparative HPLC (Column A, H₂O+0.1% HCOOH/MeCN+0.1% HCOOH 70:30 to 30:70) and freeze dried with water to obtain Example 135 (3 mg, 17%) as a white solid.

¹H-NMR (DMSO-d₆, 300 MHz) δ: 1.39 (s, 6H, 2CH₃); 1.50 (s, 6H, 2CH₃); 2.24 (s, 3H, CH₃); 2.35 (s, 3H, CH₃); 3.54- 3.59 (m, 2H, N—CH₂); 3.78 (s, 3H, CH₃); 3.81-3.85 (m, 2H, N—CH₂); 3.90-3.92 (m, 4H, 2N—CH₂); 6.37 (s, 1H, Ar); 694-6.98 (m, 2H, 2Ar); 7.42 (d, J 8.4 Hz, 1H, Ar); 7.75-7.81 (m, 1H, Ar); 8.35-8.38 (m, 1H, Ar); 8.55 (d, J 8.4 Hz, 1H, Ar).

M/Z (M+H)⁺: 529.5.

### Compound 162: 1′-(tert-butyl) 3-ethyl 5′,6′-dihydro-[2,4′-bipyridine]-1′,3′(2′H)-dicarboxylate

To a solution of 1-(tert-butyl) 3-ethyl 4-(4,4,5,5-tetramethyl-1,3,2-dioxaborolan-2-yl)-5,6-dihydropyridine-1,3(2H)-dicarboxylate (579 mg) in dioxane (12 mL) was added 2-bromopyridine (200 mg) and potassium carbonate (1.2 M in water, 3.16 mL). The reaction was sparged with argon for 10 min then Pd(dppf)Cl₂ (93 mg) was added and the reaction mixture was heated at 100° C. for 16 hour. The reaction mixture was quenched with NH4Cl sat. (50 mL) then extracted with EtOAc (2*50 mL). The combined organic layers were washed with brine (50 mL), dried over MgSO₄ and concentrated under reduced pressure. The crude was purified by flash chromatography (Interchim® 50 μm, DCM 100% to DCM/MeOH 98:2) to obtain Compound 162 (250 mg, 60%) as a yellow oil.

M/Z (M+H)⁺: 333.7.

### Compound 163: ethyl 1′,2′,5′,6′-tetrahydro-[2,4′-bipyridine]-3′-carboxylate hydrochloride

Compound 163 was prepared according to general procedure (XII) starting from Compound 162 (250 mg) using HCl in Et₂O. The crude Compound 163 was obtained as a yellow solid (250 mg) and directly engaged in the following step.

M/Z (M+H)⁺: 233.7.

### Compound 164: ethyl (cis)-4-(pyridin-2-yl)piperidine-3-carboxylate

A solution of of Compound 163 (250 mg) in EtOH (7.5 mL) was sparged with Argon for 5 minutes then Palladium on Charcoal (10% w/w, 0.1 eq) was added. The reaction mixture was stirred under a hydrogen atmosphere (1 Bar) for 48 hours, then filtered over a Celite pad which was rinsed with MeOH (50 mL). The filtrate was concentrated under reduced pressure to obtain Compound 164 (280 mg) as a yellow oil.

M/Z (M+H)⁺: 235.1.

### Example 136: ethyl 1-(1-(4-chloro-3-fluorophenyl)-3,3-dimethyl-2,3-dihydro-1H-pyrrolo[3,2-b]pyridine-5-carbonyl)-4-(pyridin-2-yl)piperidine-3-carboxylate

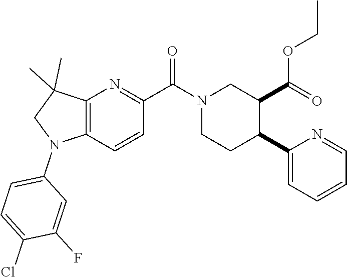

Example 136 was prepared according to general procedure (VIIIb) starting from Compound 68 (93 mg), and Compound 164 (1.1 eq.), and N,N-diisopropylethylamine (5.0 eq.) in MeTHF. The crude Example 136 (30 mg) was obtained as yellow solid and directly engaged in the following step.

M/Z (M[³⁵Cl]+H)⁺: 537.9

### Example 137 (cis)-1-(1-(4-chloro-3-fluorophenyl)-3,3-dimethyl-2,3-dihydro-1H-pyrrolo[3,2-b]pyridine-5-carbonyl)-4-(pyridin-2-yl)piperidine-3-carboxylic acid

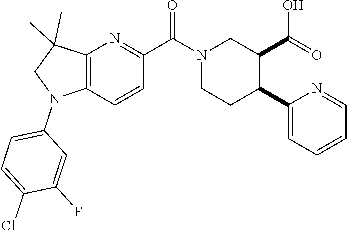

### And Example 138: (trans)-1-(1-(4-chloro-3-fluorophenyl)-3,3-dimethyl-2,3-dihydro-1H-pyrrolo[3,2-b]pyridine-5-carbonyl)-4-(pyridin-2-yl)piperidine-3-carboxylic acid

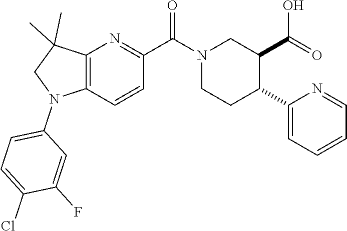

Example 137 and Example 138 were prepared according to general procedure (VII) starting from Example 136 (30 mg). The crude was purified by preparative HPLC (Column A, H₂O+0.1% HCOOH/MeCN+0.1% HCOOH 80:20 to 40:60) and freeze dried with water to obtain Example 137 (3 mg, 2% over 4 steps) as a white solid and Example 138 (5 mg, 4% over 4 steps) as a white solid.

### Example 137

¹H-NMR (DMSO-d₆, 300 MHz) δ: 1.34 (s, 6H, CH₃×2); 1.94-2.10 (m, 1H); 2.24-2.39 (m, 1H); 3.05-3.22 (m, 2H); 3.57-3.66 (m, 1H); 3.68-3.93 (m, 4H); 4.24-4.55 (m, 1H); 7.15-7.17 (m, 1H, Ar); 7.18-7.36 (m, 3H, 3Ar); 7.36-7.51 (m, 1H, Ar); 7.52-7.58 (m, 2H, 2Ar); 7.71-7.86 (bs, 1H, Ar); 8.41-8.54 (bs, 1H, Ar); COOH was not observed.

M/Z (M[³⁵Cl]+H)⁺: 509.4

### Example 138

¹H-NMR (DMSO-d₆, 300 MHz) δ: 1.18-1.42 (m, 6H, CH₃×2); 1.57-1.95 (m, 2H, pyr-CH—CH₂—CH₂); 2.80-3.10 (m, 2H); 3.10-3.22 (m, 2H); 3.79-3.93 (m, 2H, Me₂C—CH₂—N—Ar); 4.13-4.26 (m, 0.5H); 4.53-4.72 (m, 1H); 4.72-4.85 (m, 0.5H); 6.41-6.66 (bs, 0.3H); 7.14-7.27 (m, 2H, Ar×2); 7.27-7.39 (m, 2H, Ar×2); 7.39-7.50 (m, 1H, Ar); 7.50-7.62 (m, 2H, Ar×2); 7.73 (t, J 7.4 Hz, 1H, Ar); 8.48-8.50 (m, 2H, 2Ar); 11.86-12.66 (bs, 1H, COOH).

M/Z (M[³⁵Cl]+H)⁺: 509.3

### Compound 165: tert-butyl 4-(6-(methoxycarbonyl)-5-methylpyridin-2-yl)-2,2-dimethylpiperazine-1-carboxylate

Compound 165 was prepared according to general procedure (Xb), starting from methyl 6-chloro-3-methylpicolinate (217 mg) and tert-butyl 2,2-dimethylpiperazine-1-carboxylate (1.2 eq.). The crude Compound 165 (585 mg) was obtained as a yellow oil and engaged directly in the following step.

M/Z (M+H)⁺: 364.2

### Compound 166: methyl 6-(3,3-dimethylpiperazin-1-yl)-3-methylpicolinate

Compound 166 was prepared according to general procedure (XII) starting from Compound 165 (585 mg) in DCM using TFA. The reaction mixture was concentrated under reduced pressure to obtain the crude Compound 166 (522 mg) as a brown oil.

M/Z (M+H)⁺: 264.7

### Example 139: methyl 6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-3-methylpicolinate

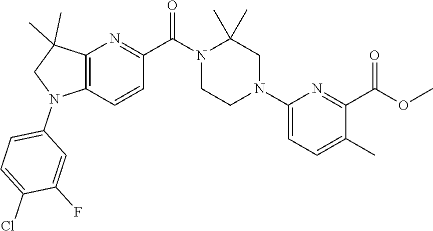 [SMILES: COC(=O)c1nc(N2CCN(C(=O)c3ccc4c(n3)C(C)(C)CN4c3ccc(Cl)c(F)c3)C(C)(C)C2)ccc1C]

Example 139 was prepared according to general procedure (XIV) starting from Compound 71 (100 mg) and Compound 166 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 50:50) to obtain Example 139 (106 mg) as an orange solid.

¹H-NMR (DMSO-d₆, 300 MHz) δ: 1.35 (s, 6H, 2CH₃); 1.49 (s, 6H, 2CH₃); 2.25 (s, 3H, CH₃); 3.51-3.57 (m, 2H N—CH₂); 3.81-3.86 (m, 9H, OCH₃+3N—CH₂); 6.79 (d, J 8.7 Hz, 1H, Ar); 7.18 (dd, J 8.7, 2.9 Hz, 1H, Ar); 7.30-7.37 (m, 2H, 2Ar); 7.37-7.58 (m, 3H, 3Ar);

M/Z (M[³⁵Cl]+H)⁺: 566.4.

### Example 140: 6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-3-methylpicolinic acid

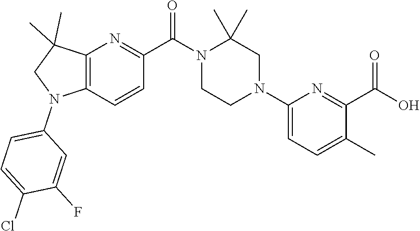 [SMILES: Cc1ccc(N2CCN(C(=O)c3ccc4c(n3)C(C)(C)CN4c3ccc(Cl)c(F)c3)C(C)(C)C2)nc1C(=O)O]

Example 140 was prepared according to general procedure (XI) starting from Example 139 (106 mg). The crude was purified by preparative HPLC (Column B, H₂O+0.1% HCOOH/MeCN+0.1% HCOOH 65:35 to 0:100) and freeze dried with HCl (0.1N in water) to obtain Example 140 (39 mg, 21%) as a yellow solid.

¹H-NMR (DMSO-d₆+D₂O, 300 MHz) δ: 1.33 (s, 6H, 2CH₃); 1.48 (s, 6H, 2CH₃); 2.31 (s, 3H, CH₃); 3.52-3.59 (m, 2H N—CH₂); 3.78-3.81 (m, 6H, 3N—CH₂); 6.84 (d, J 8.8 Hz, 1H, Ar); 7.16 (dd, J 8.8, 2.9 Hz, 1H, Ar); 7.25-7.35 (m, 2H, 2 Ar); 7.52-7.56 (m, 3H, 3Ar); COOH was not observed.

M/Z (M[³⁵Cl]+H)⁺: 552.4.

### Compound 167: tert-butyl 4-(6-methoxy-5-(methoxycarbonyl)pyridin-3-yl)-2,2-dimethylpiperazine-1-carboxylate

Compound 167 was prepared according to general procedure (Xb), starting from methyl 5-chloro-2-methoxynicotinate (287 mg) and tert-butyl 2,2-dimethylpiperazine-1-carboxylate (1.2 eq.). The crude Compound 167 (630 mg) was obtained as a brown oil and engaged directly in the following step.

M/Z (M+H)⁺: 364.2

### Compound 168: methyl 5-(3,3-dimethylpiperazin-1-yl)-2-methoxynicotinate

Compound 168 was prepared according to general procedure (XII) starting from Compound 167 (585 mg) in DCM using TFA. The reaction mixture was concentrated under reduced pressure to obtain the crude Compound 168 (198 mg) as a brown oil.

M/Z (M+H)⁺: 264.7

### Example 141: methyl 5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-2-methoxynicotinate

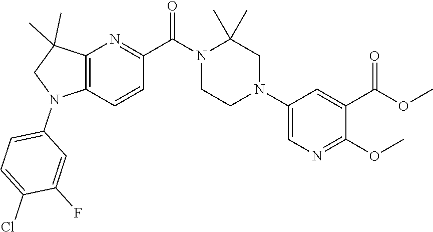 [SMILES: COC(=O)c1cc(N2CCN(C(=O)c3ccc4c(n3)C(C)(C)CN4c3ccc(Cl)c(F)c3)C(C)(C)C2)cnc1OC]

Example 141 was prepared according to general procedure (XIV) starting from Compound 71 (100 mg) and Compound 168 (1.9 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 45:55) to obtain Example 141 (80 mg) as a yellow oil.

¹H-NMR (DMSO-d₆, 300 MHz) δ: 1.34 (s, 6H, 2CH₃); 1.51 (s, 6H, 2CH₃); 3.32-3.40 (m, 4H, 2N—CH₂); 3.73-3.78 (m, 2H, N—CH₂); 3.77 (s, 3H, O—CH₃); 3.79 (s, 3H, O—CH₃); 3.82-3.85 (s, 2H, N—CH₂); 7.16-7.20 (m, 1H, Ar); 7.30-7.36 (m, 2H, 2Ar); 7.51-7.59 (m, 3H, 3Ar); 7.93-7.94 (m, 1H, Ar).

M/Z (M[³⁵Cl]+H)⁺: 582.5.

### Example 142: 5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-2-methoxynicotinic acid

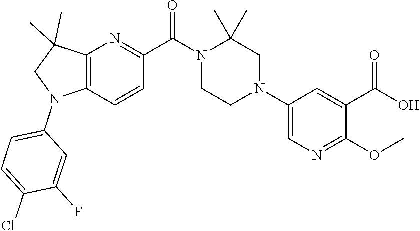 [SMILES: COc1ncc(N2CCN(C(=O)c3ccc4c(n3)C(C)(C)CN4c3ccc(Cl)c(F)c3)C(C)(C)C2)cc1C(=O)O]

Example 142 was prepared according to general procedure (XI) starting from Example 141 (80 mg). The crude was purified by preparative HPLC (Column B, H₂O+0.1% HCOOH/MeCN+0.1% HCOOH 65:35 to 0:100) and freeze dried with HCl (0.1N in water) to obtain Example 142 (29 mg, 15%) as a yellow solid.

¹H-NMR (DMSO-d₆+D₂O, 300 MHz) δ: 1.35 (s, 6H, 2CH₃); 1.52 (s, 6H, 2CH₃); 3.20-3.25 (m, 1H, N—CHₐH_{b}); 3.34-3.42 (m, 3H, N—CHₐH_{b}+O-CH₃); 3.72-3.78 (m, 2H, N—CH₂); 3.82 (s, 2H, N—CH₂); 3.86 (s, 2H, N—CH₂); 7.16-7.22 (m, 1H, Ar); 7.30-7.38 (m, 2H, 2Ar); 7.52-7.59 (m, 3H, 3Ar); 7.91-8.20 (m, 1H, Ar); COOH was not observed.

M/Z (M[³⁵Cl]+H)⁺: 568.4.

### Compound 169: (3R)1-(3-chloropyridin-2-yl)-3-methoxycyclopentane-1-carbonitrile

To a solution of 2-(3-chloropyridin-2-yl)acetonitrile (935 mg) and (R)-2-methoxybutane-1,4-diyl dimethanesulfonate (1.0 eq) in THE (30 mL) at 0° C. was added Potassium tert-butoxide (2.0 eq). The reaction mixture was stirred at 25° C. for 18 hours, quenched with NH₄Cl (sat. aq. 10 mL), diluted with water (5 mL) and extracted with EtOAc (3*10 mL).

The combined organic layers were washed with brine (10 mL), dried over MgSO₄, and concentrated under reduced pressure. The crude was purified by flash chromatography (Interchim® 50 μm, CyHex 100% to CyHex/EtOAc 60:40) to obtain Compound 169 (510 mg) as a clear oil.

M/Z (M[³⁵Cl]+H)⁺: 237.1

### Compound 170: ((3R)-1-(3-chloropyridin-2-yl)-3-methoxycyclopentyl)methanamine

Compound 170 (263 mg, 51%) was obtained as a clear oil following general procedure (XVa), starting from Compound 169 (510 mg).

M/Z (M[³⁵Cl]+H⁺): 241.1

### Compound 171: (3R)-1′-(4-chloro-3-fluorophenyl)-3-methoxy-1′,2′-dihydrospiro[cyclopentane-1,3′-pyrrolo[3,2-b]pyridine]

Compound 171 was prepared according to general procedure (Xc) from Compound 170 (450 mg) and 4-bromo-1-chloro-2-fluorobenzene. The crude was purified by flash chromatography (Interchim® 50 μm, CyHex 100% to CyHex/EtOAc 60:40) to obtain Compound 171 (440 mg, 71%) as a yellow oil.

M/Z (M[³⁵Cl]+H⁺): 333.2

### Compound 172: (major diastereoisomere) (3R)-5′-bromo-1′-(4-chloro-3-fluorophenyl)-3-methoxy-1′,2′-dihydrospiro[cyclopentane-1,3′-pyrrolo[3,2-b]pyridine]

### And Compound 173: (minor diastereoisomere) (3R)-5′-bromo-1′-(4-chloro-3-fluorophenyl)-3-methoxy-1′,2′-dihydrospiro[cyclopentane-1,3′-pyrrolo[3,2-b]pyridine]

Compound 172 and Compound 173 were prepared according to general procedure (XVI) in MeCN from Compound 171 (440 mg). The crude was purified by flash chromatography (Interchim® 50 μm, CyHex 100% to CyHex/EtOAc 85:15) to obtain Compound 172 (330 mg, 61%) as a white solid and Compound 173 (140 mg, 26%) as a yellow oil.

Compound 172: M/Z (M[³⁵Cl][⁸¹Br]+H)⁺: 413.1.

Compound 173: M/Z (M[³⁵Cl][⁸¹Br]+H)⁺: 413.1.

### Example 143: (major diastereoisomere) methyl 6-(4-((3R)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinate

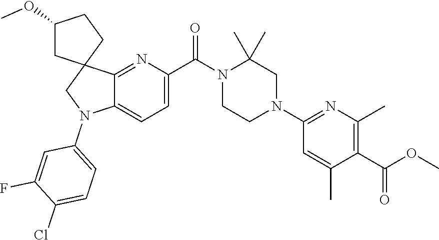

Example 143 was prepared according to general procedure (XIV) starting from Compound 172 (100 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 20:80) to obtain Example 143 (78 mg, 51%) as a white solid.

¹H-NMR (CDCl₃, 300 MHz) δ: 1.58 (s, 6H, (CH₃)₂); 1.80-2.31 (m, 6H, 3CH₂); 2.36 (s, 3H, CH₃); 2.51 (s, 3H, CH₃); 3.35 (s, 3H, C(O)OCH₃); 3.61-3.65 (m, 2H, N—CH₂); 3.88 (s, 3H, OCH₃); 3.89-4.06 (m, 6H, 3N—CH₂); 4.10-4.13 (m, 1H, CH—OMe); 6.10 (s, 1H, Ar); 6.92-7.03 (m, 2H, 2Ar); 7.27-7.41 (m, 2H, 2Ar); 7.50 (d, J 7.2 Hz, 1H).

M/Z (M[³⁵Cl]+H)⁺: 636.3.

### Example 144: (major diastereoisomere) 6-(4-((3R)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid

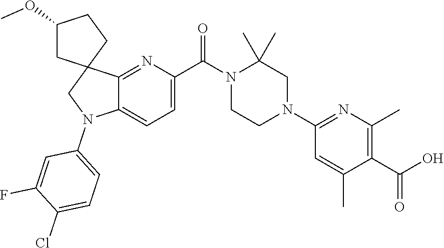 [SMILES: CO[C@@H]1CCC2(C1)CN(c1ccc(Cl)c(F)c1)c1ccc(C(=O)N3CCN(c4cc(C)c(C(=O)O)c(C)n4)CC3(C)C)nc12]

Example 144 was prepared according to general procedure (XI) starting from Example 143 (75 mg). The crude was purified by preparative HPLC (Column B, H₂O+0.1% HCOOH/MeCN+0.1% HCOOH 65:35 to 0:100) and freeze dried with HCl (0.1N in water) to obtain Example 144 (58 mg, 75%) as a yellow solid.

¹H-NMR (DMSO-d₆, 300 MHz) δ: 1.51 (s, 6H, (CH₃)₂); 1.80-2.02 (m, 4H, (CH₂)₂); 2.08-2.22 (m, 2H, CH₂); 2.32 (s, 3H, CH₃); 3.23 (s, 3H, CH₃); 3.60-3.66 (m, 2H, CH₂); 3.83-3.91 (m, 4H, (CH₂)₂); 3.95-4.09 (m, 4H, 2N—CH₂); 6.65 (bs, 1H, Ar); 7.16 (dd, J 9.0, 2.1 Hz, 1H, Ar); 7.30-7.38 (m, 2H, 2Ar); 7.52-7.58 (m, 2H, 2Ar). One CH₃ and COOH were not observed.

M/Z (M[³⁵Cl]+H)⁺: 622.4

### Compound 174: (3S)1-(3-chloropyridin-2-yl)-3-methoxycyclopentane-1-carbonitrile

To a solution of 2-(3-chloropyridin-2-yl)acetonitrile (1.74 g) and (S)-2-methoxybutane-1,4-diyl dimethanesulfonate (1.0 eq) in THE (30 mL) at 0° C. was added Potassium tert-butoxide (2.0 eq). The reaction mixture was stirred at 25° C. for 18 hours, quenched with NH₄Cl (sat. aq. 10 mL), diluted with water (5 mL) and extracted with EtOAc (3*10 mL). The combined organic layers were washed with brine (10 mL), dried over MgSO₄, and concentrated under reduced pressure. The crude was purified by flash chromatography (Interchim® 50 μm, CyHex 100% to CyHex/EtOAc 60:40) to obtain Compound 174 (760 mg, 28%) as a clear oil.

M/Z (M[³⁵Cl]+H)⁺: 237.6

### Compound 175: ((3S)-1-(3-chloropyridin-2-yl)-3-methoxycyclopentyl)methanamine

Compound 175 (540 mg, 70%) was obtained as a clear oil following general procedure (XVa), starting from Compound 174 (760 mg).

M/Z (M[³⁵Cl]+H⁺): 241.1

### Compound 176: (3S)-1′-(4-chloro-3-fluorophenyl)-3-methoxy-1′,2′-dihydrospiro[cyclopentane-1,3′-pyrrolo[3,2-b]pyridine]

Compound 176 was prepared according to general procedure (Xc) from Compound 175 (520 mg) and 4-bromo-1-chloro-2-fluorobenzene. The crude was purified by flash chromatography (Interchim® 50 μm, CyHex 100% to CyHex/EtOAc 60:40) to obtain Compound 176 (350 mg, 48%) as a yellow oil.

M/Z (M[³⁵Cl]+H⁺): 333.2

### Compound 177: (major diastereoisomere) (3S)-5′-bromo-1′-(4-chloro-3-fluorophenyl)-3-methoxy-1′,2′-dihydrospiro[cyclopentane-1,3′-pyrrolo[3,2-b]pyridine]

### And Compound 178: (minor diastereoisomere) (3S)-5′-bromo-1′-(4-chloro-3-fluorophenyl)-3-methoxy-1′,2′-dihydrospiro[cyclopentane-1,3′-pyrrolo[3,2-b]pyridine]

Compound 177 and Compound 178 were prepared according to general procedure (XVI) in MeCN from Compound 176 (335 mg). The crude was purified by flash chromatography (Interchim® 50 μm, CyHex 100% to CyHex/EtOAc 85:15) to obtain Compound 177 (250 mg, 60%) as a yellow oil and Compound 178 (90 mg, 22%) as a yellow oil.

Compound 177: M/Z (M[³⁵Cl][⁸¹Br]+H)⁺: 413.1.

Compound 178: M/Z (M[³⁵Cl][⁸¹Br]+H)⁺: 413.1.

### Example 145: (major diastereoisomere) methyl 6-(4-((3S)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinate

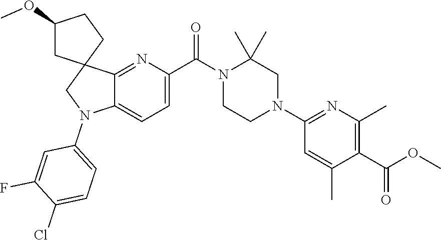

Example 145 was prepared according to general procedure (XIV) starting from Compound 177 (100 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 20 μm, CyHex 100% to CyHex/EtOAc 20:80) to obtain Example 145 (104 mg, 67%) as a white solid.

¹H-NMR (CDCl₃, 300 MHz) δ: 1.58 (s, 6H, (CH₃)₂); 1.80-2.31 (m, 6H, 3CH₂); 2.36 (s, 3H, CH₃); 2.50 (s, 3H, CH₃); 3.35 (s, 3H, C(O)OCH₃); 3.61-3.65 (m, 2H, N—CH₂); 3.88 (s, 3H, OCH₃); 3.89-4.06 (m, 6H, 3N—CH₂); 4.10-4.13 (m, 1H, CH—OMe); 6.10 (s, 1H, Ar); 6.92-7.03 (m, 2H, 2Ar); 7.27-7.41 (m, 2H, 2Ar); 7.50 (d, J 7.2 Hz, 1H).

M/Z (M[³⁵Cl]+H)⁺: 636.3.

### Example 146: (major diastereoisomere) 6-(4-((3S)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid

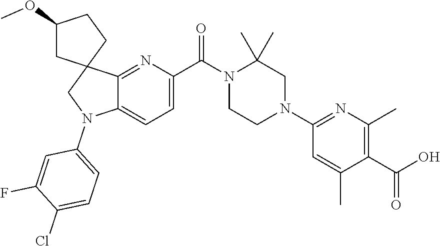 [SMILES: CO[C@H]1CCC2(C1)CN(c1ccc(Cl)c(F)c1)c1ccc(C(=O)N3CCN(c4cc(C)c(C(=O)O)c(C)n4)CC3(C)C)nc12]

Example 146 was prepared according to general procedure (XI) starting from Example 145 (100 mg). The crude was purified by preparative HPLC (Column B, H₂O+0.1% HCOOH/MeCN+0.1% HCOOH 65:35 to 0:100) and freeze dried with HCl (0.1N in water) to obtain Example 146 (65 mg, 63%) as a yellow solid.

¹H-NMR (DMSO-d₆+D₂O, 300 MHz) δ: 1.50 (s, 6H, (CH₃)₂); 1.80-2.02 (m, 4H, (CH₂)₂); 2.08-2.22 (m, 2H, CH₂); 2.32 (s, 3H, CH₃); 2.49 (s, 3H, CH₃); 3.23 (s, 3H, CH₃); 3.60-3.66 (m, 2H, CH₂); 3.83-3.91 (m, 4H, 2N—CH₂); 3.95-4.09 (m, 4H, 2N—CH₂); 6.65 (bs, 1H, Ar); 7.16 (dd, J 9.0, 2.1 Hz, 1H, Ar); 7.30-7.38 (m, 2H, 2Ar); 7.52-7.58 (m, 2H, 2Ar). COOH was not observed.

M/Z (M[³⁵Cl]+H)⁺: 622.4

### Example 147: methyl 2-(6-(4-(1-(4-chloro-3-fluorophenyl)-3,3-diethyl-2,3-dihydro-1H-pyrrolo[3,2-b]pyridine-5-carbonyl)-3,3-dimethylpiperazin-1-yl)pyridin-3-yl)acetate

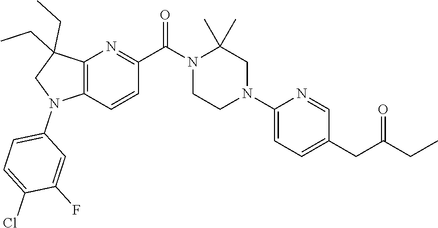 [SMILES: CCC(=O)Cc1ccc(N2CCN(C(=O)c3ccc4c(n3)C(CC)(CC)CN4c3ccc(Cl)c(F)c3)C(C)(C)C2)nc1]

Example 147 was prepared according to general procedure (XIV) starting from Compound 132 (100 mg) and Compound 111 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 147 (93 mg) as a yellow oil.

M/Z (M[³⁵Cl]+H)⁺: 594.6.

### Example 148: 2-(6-(4-(1-(4-chloro-3-fluorophenyl)-3,3-diethyl-2,3-dihydro-1H-pyrrolo[3,2-b]pyridine-5-carbonyl)-3,3-dimethylpiperazin-1-yl)pyridin-3-yl)acetic acid

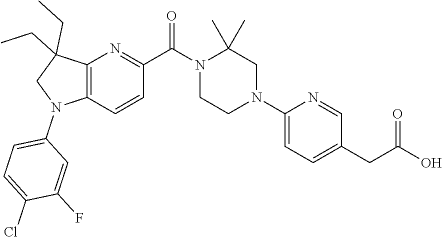 [SMILES: CCC1(CC)CN(c2ccc(Cl)c(F)c2)c2ccc(C(=O)N3CCN(c4ccc(CC(=O)O)cn4)CC3(C)C)nc21]

Example 148 was prepared according to general procedure (VII) starting from Example 147 (90 mg). The crude was purified by preparative HPLC (Column B, H₂O+0.1% HCOOH/MeCN+0.1% HCOOH 65:35 to 0:100) and freeze dried with HCl (0.1N in water) to obtain Example 148 (38 mg, 26% over 2 steps) as a yellow solid.

¹H-NMR (DMSO-d₆, 300 MHz) δ: 0.76 (t, J 7.2 Hz, 6H, 2CH₂—CH₃); 1.53 (s, 6H, 2CH₃); 1.68-1.76 (m, 4H, 2CH₂CH₃); 3.55-3.65 (m, 4H, 2-N—CH₂); 3.82-3.93 (m, 6H, CH₂—CO, 2N—CH₂); 6.90-7.23 (m, 2H, 2Ar); 7.33-7.40 (m, 2H, 2Ar); 7.51-7.56 (m, 2H, 2Ar); 7.81 (bs, 1H, Ar); 7.94 (s, 1H, Ar); 12.53 (bs, 1H, CO₂H).

M/Z (M[³⁵Cl]+H)⁺: 580.4

### Compound 179: tert-butyl 4-(5-(methoxycarbonyl)pyridin-3-yl)-2,2-dimethylpiperazine-1-carboxylate

Compound 179 was prepared according to general procedure (Xb), starting from methyl 5-chloronicotinate (296 mg) and tert-butyl 2,2-dimethylpiperazine-1-carboxylate (1.2 eq.). The crude Compound 179 (550 mg) was obtained as a yellow oil and engaged directly in the following step.

M/Z (M+H)⁺: 350.2

### Compound 180: methyl 5-(3,3-dimethylpiperazin-1-yl)nicotinate

Compound 180 was prepared according to general procedure (XII) starting from Compound 179 (550 mg) in DCM using TFA. The reaction mixture was concentrated under reduced pressure to obtain the crude Compound 180 (477 mg) as a brown oil.

M/Z (M+H)⁺: 250.2

### Example 149: methyl 5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)nicotinate

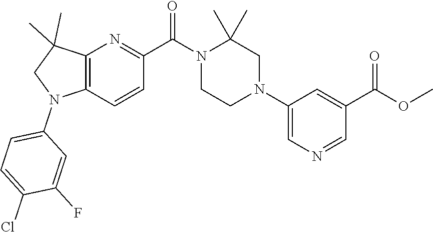 [SMILES: COC(=O)c1cncc(N2CCN(C(=O)c3ccc4c(n3)C(C)(C)CN4c3ccc(Cl)c(F)c3)C(C)(C)C2)c1]

### And Example 150: methyl 5-(4-(1-(3-fluoro-4-(4-(5-(methoxycarbonyl)pyridin-3-yl)-2,2-dimethylpiperazine-1-carbonyl)phenyl)-3,3-dimethyl-2,3-dihydro-1H-pyrrolo[3,2-b]pyridine-5-carbonyl)-3,3-dimethylpiperazin-1-yl)nicotinate

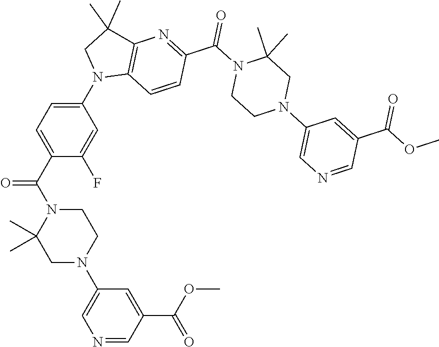 [SMILES: COC(=O)c1cncc(N2CCN(C(=O)c3ccc4c(n3)C(C)(C)CN4c3ccc(C(=O)N4CCN(c5cncc(C(=O)OC)c5)CC4(C)C)c(F)c3)C(C)(C)C2)c1]

Example 149 and Example 150 were prepared according to general procedure (XIV) starting from Compound 71 (100 mg) and Compound 180 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, DCM 100% to DCM/MeOH 98:2) to obtain Example 149 (30 mg, 17%) as a yellow oil and Example 150 as a yellow solid (89 mg, 35%).

### Example 149

¹H-NMR (DMSO-d₆, 300 MHz) δ: 1.34 (s, 6H, 2CH₃); 1.53 (s, 6H, 2CH₃); 3.34-3.52 (m, 4H, N—CH₂); 3.62-3.66 (m, 2H, N—CH₂); 3.80-3.88 (m, 5H, O—CH₃+N—CH₂); 7.16-7.21 (m, 1H, Ar); 7.33-7.38 (m, 2H, 2Ar); 7.38-7.42 (m, 2H, 2 Ar); 7.44-7.52 (m, 1H, Ar); 7.57 (d, J 8.4 Hz, 1H, Ar); 8.37-8.42 (m, 1H, Ar).

M/Z (M[³⁵Cl]+H)⁺: 552.4.

### Example 150

¹H-NMR (DMSO-d₆, 300 MHz) δ: 1.37 (s, 6H, 2CH₃); 1.53 (s, 12H, 2*2CH₃); 3.33-3.52 (m, 4H, 2N—CH₂); 3.62-3.72 (m, 6H, 3N—CH₂); 3.83-3.93 (m, 10H, 2O—CH₃+2N—CH₂); 7.14-7.23 (m, 2H, 2Ar); 7.37-7.43 (m, 2H, 2Ar); 7.48-7.52 (m, 2H, 2Ar); 7.63 (d, J 8.4 Hz, 1H, Ar); 8.38-8.42 (m, 4H, 4Ar).

M/Z (M+H)⁺: 765.6.

### Example 151: 5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)nicotinic acid

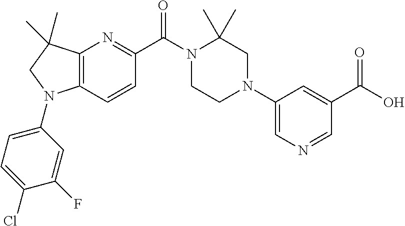 [SMILES: CC1(C)CN(c2ccc(Cl)c(F)c2)c2ccc(C(=O)N3CCN(c4cncc(C(=O)O)c4)CC3(C)C)nc21]

Example 151 was prepared according to general procedure (XI) starting from Example 149 (90 mg). The crude was purified by preparative HPLC (Column B, H₂O+0.1% HCOOH/MeCN+0.1% HCOOH 95:05 to 60:40) and freeze dried with HCl (0.1N in water) to obtain Example 151 (21 mg, 23%) as a yellow solid.

¹H-NMR (CD₃OD, 300 MHz) δ: 1.33 (s, 6H, 2CH₃); 1.56 (s, 6H, 2CH₃); 3.55 (t, J 5.5 Hz, 2H, N—CH₂); 3.72 (s, 2H, N—CH₂); 3.76 (s, 2H, N—CH₂); 3.92 (t, J 5.5 Hz, 2H, N—CH₂); 7.02-7.06 (m, 1H, Ar); 7.08-7.13 (m, 1H, Ar); 7.30-7.39 (m, 2H, 2Ar); 7.44 (d, J 8.5 Hz, 1H, Ar); 8.21-8.22 (m, 1H, Ar); 8.31-8.32 (m, 1H, Ar); 8.38 (s, 1H, Ar). COOH was not observed.

M/Z (M[³⁵Cl]+H)⁺: 538.3.

### Example 152: 5-(4-(4-(5-(4-(5-carboxypyridin-3-yl)-2,2-dimethylpiperazine-1-carbonyl)-3,3-dimethyl-2,3-dihydro-1H-pyrrolo[3,2-b]pyridin-1-yl)-2-fluorobenzoyl)-3,3-dimethylpiperazin-1-yl)nicotinic acid

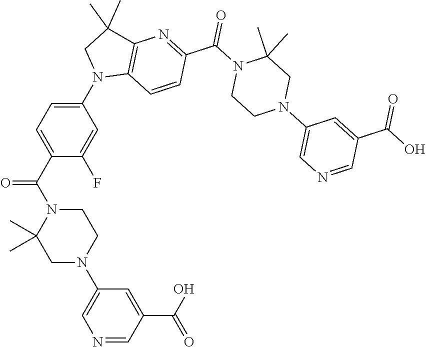 [SMILES: CC1(C)CN(c2ccc(C(=O)N3CCN(c4cncc(C(=O)O)c4)CC3(C)C)c(F)c2)c2ccc(C(=O)N3CCN(c4cncc(C(=O)O)c4)CC3(C)C)nc21]

Example 152 was prepared according to general procedure (XI) starting from Example 150 (89 mg). The crude was purified by preparative HPLC (Column B, H₂O+0.1% HCOOH/MeCN+0.1% HCOOH 65:35 to 0:100) and freeze dried with HCl (0.1N in water) to obtain Example 152 (40 mg, 43%) as a yellow solid.

¹H-NMR (CD₃OD, 300 MHz) δ: 1.37 (s, 6H, 2CH₃); 1.54-1.55 (m, 12H, 4CH₃); 3.49 (t, J 5.5 Hz, 2H, N—CH₂); 3.55 (t, J 5.5 Hz, 2H, N—CH₂); 3.70-3.85 (m, 8H, 4N—CH₂); 3.93 (t, J 5.5 Hz, 2H, N—CH₂); 7.05 (dd, J 12.2 2.2 Hz, 1H, Ar); 7.16 (dd, J 8.4 2.2 Hz, 1H, Ar); 7.32-7.37 (m, 1H, Ar); 7.41 (d, J 8.4 Hz, 1H, Ar); 7.6 (d, J 8.4 Hz, 1H, Ar); 8.18-8.20 (m, 2H, 2Ar); 8.30-8.33 (m, 2H, 2Ar); 8.38 (bs, 2H, 2Ar). 2COOH were not observed.

M/Z (M+H)⁺: 765.6.

### Compound 181: 1-(3-chloropyridin-2-yl)-3-methylenecyclobutane-1-carbonitrile

Compound 181 was prepared according to general procedure (I) from 3-chloro-2-fluoropyridine (1.0 g) and 3-methylenecyclobutane-1-carbonitrile. The crude was purified by flash chromatography (Interchim® 50 μm, CyHex 100% to CyHex/EtOAc 80:20) to obtain Compound 181 (0.98 g, 63%) as a clear oil.

M/Z (M[³⁵Cl]+H⁺): 205.6

### Compound 182: (1-(3-chloropyridin-2-yl)-3-methylcyclobutyl)methanamine

Compound 182 (587 mg, 59%) was obtained as a yellow oil following general procedure (XVa), starting from Compound 181 (978 mg).

M/Z (M[³⁵Cl]+H⁺): 211.6.

### Compound 183: 1′-(4-chloro-3-fluorophenyl)-3-methyl-1′,2′-dihydrospiro[cyclobutane-1,3′-pyrrolo[3,2-b]pyridine]

Compound 183 was prepared according to general procedure (Xc) from Compound 182 (585 mg) and 4-bromo-1-chloro-2-fluorobenzene. The crude was purified by flash chromatography (Interchim® 50 μm, CyHex 100% to EtOAc 100%) to obtain Compound 183 (301 mg, 36%) as a white solid.

M/Z (M[³⁵Cl]+H⁺): 303.3

### Compound 184: (major diastereoisomere) 5′-bromo-1′-(4-chloro-3-fluorophenyl)-3-methyl-1′,2′-dihydrospiro[cyclobutane-1,3′-pyrrolo[3,2-b]pyridine]

### And Compound 185: (minor diastereoisomere) 5′-bromo-1′-(4-chloro-3-fluorophenyl)-3-methyl-1′,2′-dihydrospiro[cyclobutane-1,3′-pyrrolo[3,2-b]pyridine]

Compound 184 and Compound 185 were prepared according to general procedure (XVI) in MeCN from Compound 183 (301 mg). The crude was purified by flash chromatography (Interchim® 20 μm, CyHex 100% to CyHex/EtOAc 85:15) to obtain Compound 184 (231 mg, 61%) as a white solid and Compound 185 (71 mg, 19%) as a white solid.

Compound 184: M/Z (M[³⁵Cl][⁸¹Br]+H)⁺: 383.1

Compound 185: M/Z (M[³⁵Cl][⁸¹Br]+H)⁺: 383.1

### Example 153: (major diastereoisomere) methyl 2-(6-(4-(1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)pyridin-3-yl)acetate

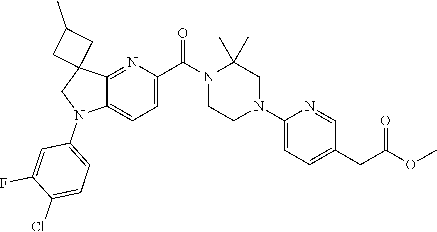 [SMILES: COC(=O)Cc1ccc(N2CCN(C(=O)c3ccc4c(n3)C3(CC(C)C3)CN4c3ccc(Cl)c(F)c3)C(C)(C)C2)nc1]

Example 153 was prepared according to general procedure (XIV) starting from Compound 184 (115 mg) and Compound 111 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 20:80) to obtain Example 153 (49 mg) as a white solid.

¹H-NMR (DMSO-d₆, 300 MHz) δ: 1.15-1.23 (m, 3H, CH₃); 1.51 (s, 6H, 2CH₃); 1.90-1.98 (m, 2H, 2CHₐH_{b}); 2.53-2.72 (m, 3H, CHMe+2CHₐH_{b}); 3.49-3.66 (m, 8H, CH₂C(O)⁺3N—CH₂); 3.86 (s, 3H, O—CH₃); 4.12 (m, 2H, N—CH₂); 6.52-6.61 (m, 1H, Ar); 7.15-7.19 (m, 1H, Ar); 7.22-7.60 (m, 5H, Ar); 7.93-7.97 (m, 1H, Ar).

M/Z (M[³⁵Cl]+H)⁺: 592.4

### Example 154: (major diastereoisomere) 2-(6-(4-(1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid

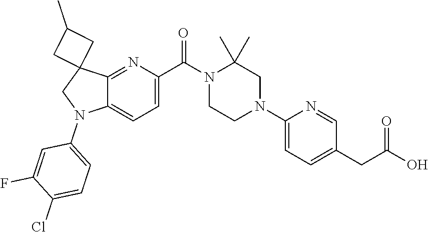 [SMILES: CC1CC2(C1)CN(c1ccc(Cl)c(F)c1)c1ccc(C(=O)N3CCN(c4ccc(CC(=O)O)cn4)CC3(C)C)nc12]

Example 154 was prepared according to general procedure (VII) starting from Example 153 (49 mg). The crude was purified by preparative HPLC (Column B, H₂O+0.1% HCOOH/MeCN+0.1% HCOOH 65:35 to 0:100) and freeze dried with HCl (0.1N in water) to obtain Example 154 (7 mg, 4% over 2 steps) as a white solid.

¹H-NMR (DMSO-d₆, 300 MHz) δ: 1.17 (d, J 6.7 Hz, 3H, CH₃); 1.54 (s, 6H, 2CH₃); 1.87-2.00 (m, 2H, CHaHb-CHMe-CHa′Hb′); 2.55-2.64 (m, 2H, CHaHb-CHMe-CHa′Hb′); 2.65-2.80 (m, 1H, CHMe); 3.50-3.60 (m, 2H, N—CH₂); 3.60-3.71 (m, 2H, N—CH₂); 3.82-4.02 (m, 2H, N—CH₂); 3.91 (s, 2H, CH₂—COOH); 4.13 (s, 2H, N—CH₂); 6.74-7.11 (m, 1H, Ar); 7.14-7.20 (m, 1H, Ar); 7.30-7.35 (m, 1H, Ar); 7.39 (d, J 8.9 Hz, 1H, Ar); 7.51-7.68 (m, 2H, 2Ar); 7.61-7.90 (m, 1H, Ar); 7.93-7.97 (m, 1H, Ar); 12.10-12.70 (m, 1H, COOH).

M/Z (M[³⁵Cl]+H)⁺: 578.5.

### Example 155: (major diastereoisomere) methyl 6-(4-(1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinate

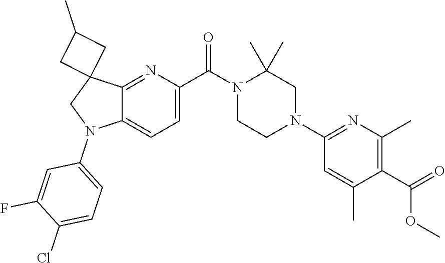 [SMILES: COC(=O)c1c(C)cc(N2CCN(C(=O)c3ccc4c(n3)C3(CC(C)C3)CN4c3ccc(Cl)c(F)c3)C(C)(C)C2)nc1C]

Example 155 was prepared according to general procedure (XIV) starting from Compound 184 (115 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 20 μm, CyHex 100% to CyHex/EtOAc 40:60) to obtain Example 155 (79 mg) as a white solid.

¹H-NMR (DMSO-d₆, 300 MHz) δ: 1.15-1.23 (m, 3H, CH₃); 1.51 (s, 6H, 2CH₃); 1.90-1.98 (m, 2H, 2CHₐH_{b}); 2.22 (s, 3H, CH₃); 2.33 (s, 3H, CH₃); 2.53-2.72 (m, 3H, CHMe+2CHₐH_{b}); 3.59-3.68 (m, 2H, N—CH₂); 3.77 (s, 3H, O—CH₃); 3.87-3.89 (m, 4H, 2N—CH₂); 4.11 (s, 2H, N—CH₂); 6.33 (s, 1H, Ar); 7.15-7.19 (m, 1H, Ar); 7.24-7.45 (m, 3H, 3Ar); 7.48-7.57 (m, 1H, Ar).

M/Z (M[³⁵Cl]+H)⁺: 606.5.

### Example 156: (major diastereoisomere) 6-(4-(1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid

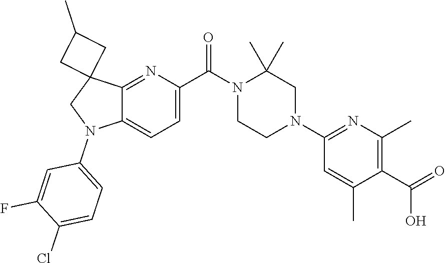 [SMILES: Cc1cc(N2CCN(C(=O)c3ccc4c(n3)C3(CC(C)C3)CN4c3ccc(Cl)c(F)c3)C(C)(C)C2)nc(C)c1C(=O)O]

Example 156 was prepared according to general procedure (XI) starting from Example 155 (79 mg). The crude was purified by preparative HPLC (Column B, H₂O+0.1% HCOOH/MeCN+0.1% HCOOH 65:35 to 0:100) and freeze dried with HCl (0.1N in water) to obtain Example 156 (37 mg, 20%) as a white solid.

¹H-NMR (DMSO-d₆, 300 MHz) δ: 1.17 (d, J 7.0 Hz, 3H, CH—CH₃); 1.52 (s, 6H, 2CH₃); 1.88-2.00 (m, 2H, CHₐH_{b}); 2.28 (s, 3H, Ar—CH₃); 2.42 (s, 3H, Ar—CH₃); 2.54-2.62 (m, 2H, CHₐH_{b}); 2.68-2.84 (m, 1H, CH-Me); 3.59-3.72 (m, 2H, N—CH₂); 3.91-4.00 (m, 4H, 2N—CH₂); 4.12 (s, 2H, N—CH₂); 6.27-6.62 (m, 1H, Ar); 7.18 (dd, J 9.2, 2.3 Hz, 1H, Ar); 7.32 (dd, J 11.8, 2.3 Hz, 1H, Ar); 7.39 (d, J 8.67 Hz, 1H, Ar); 7.50-7.59 (m, 2H, 2Ar); COOH was not observed.

M/Z (M[³⁵Cl]+H)⁺: 592.4

### Example 157: (minor diastereoisomere) methyl 6-(4-(1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinate

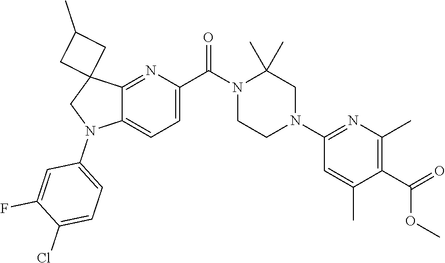 [SMILES: COC(=O)c1c(C)cc(N2CCN(C(=O)c3ccc4c(n3)C3(CC(C)C3)CN4c3ccc(Cl)c(F)c3)C(C)(C)C2)nc1C]

Example 157 was prepared according to general procedure (XIV) starting from Compound 185 (71 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 20 μm, CyHex 100% to CyHex/EtOAc 40:60) to obtain Example 157 (48 mg) as a white solid.

¹H-NMR (DMSO-d₆, 300 MHz) δ: 1.20 (d, J 6.4 Hz, 3H, CH—CH₃); 1.38 (s, 6H, 2CH₃); 2.02-2.16 (m, 2H, 2CHₐH_{b}); 2.22 (s, 3H, CH₃); 2.31-2.38 (m, 5H, CH₃+2CHₐH_{b}); 3.42-3.49 (m, 1H, CHMe); 3.59-3.70 (m, 2H, N—CH₂); 3.77 (s, 3H, O—CH₃); 3.83-3.89 (m, 4H, 2N—CH₂); 4.23 (s, 2H, N—CH₂); 6.34 (s, 1H, Ar); 7.15-7.19 (m, 1H, Ar); 7.24-7.45 (m, 3H, 3Ar); 7.48-7.57 (m, 1H, Ar).

M/Z (M[³⁵Cl]+H)⁺: 606.5.

### Example 158: (minor diastereoisomere) 6-(4-(1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid

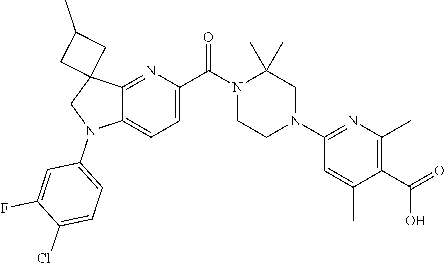 [SMILES: Cc1cc(N2CCN(C(=O)c3ccc4c(n3)C3(CC(C)C3)CN4c3ccc(Cl)c(F)c3)C(C)(C)C2)nc(C)c1C(=O)O]

Example 158 was prepared according to general procedure (XI) starting from Example 157 (79 mg). The crude was purified by preparative HPLC (Column B, H₂O+0.1% HCOOH/MeCN+0.1% HCOOH 65:35 to 0:100) and freeze dried with HCl (0.1N in water) to obtain Example 158 (37 mg, 20%) as a white solid.

¹H-NMR (DMSO-d₆, 300 MHz) δ: 1.20 (d, J 6.4 Hz, 3H, CH—CH₃); 1.53 (s, 6H, 2CH₃); 2.08-2.16 (m, 2H, CHₐH_{b}); 2.30 (s, 3H, Ar—CH₃); 2.34-2.40 (m, 2H, CHₐH_{b}); 2.46 (s, 3H, Ar—CH₃); 2.53-2.61 (m, 1H, CH-Me); 3.67-3.79 (m, 2H, N—CH₂); 3.88-3.99 (m, 4H, 2N—CH₂); 4.25 (s, 2H, N—CH₂); 6.45-6.74 (m, 1H, Ar); 7.20 (dd, J 9.1, 2.5 Hz, 1H, Ar); 7.34 (dd, J 12.0, 2.5 Hz, 1H, Ar); 7.39 (d, J 8.5 Hz, 1H, Ar); 7.51-7.59 (m, 2H, 2Ar); COOH was not observed.

M/Z (M[³⁵Cl]+H)⁺: 592.4.

### Compound 186: methyl 2-(6-chloropyridin-3-yl)propanoate

Compound 186 (410 mg, 76%) was obtained without further purification as a yellow oil, from methyl 2-(6-chloropyridin-3-yl)acetate (500 mg), according to general procedure (XIII) in THE with NaH (1.0 eq.) and Mel (1.0 eq).

M/Z (M[³⁵Cl]+H)⁺: 200.1

### Compound 187: tert-butyl 4-(5-(1-methoxy-1-oxopropan-2-yl)pyridin-2-yl)-2,2-dimethylpiperazine-1-carboxylate

Compound 187 was prepared according to general procedure (Xb), starting from Compound 186 (600 mg) and tert-butyl 2,2-dimethylpiperazine-1-carboxylate (1.2 eq.). The crude Compound 187 (780 mg) was obtained as a yellow solid and engaged directly in the following step.

M/Z (M+H)⁺: 378.1

### Compound 188: methyl 2-(6-(3,3-dimethylpiperazin-1-yl)pyridin-3-yl)propanoate hydrochloride

Compound 188 was prepared according to general procedure (XII) starting from Compound 187 (780 mg) in DCM using TFA. The reaction mixture was concentrated under reduced pressure then purified by Ion Exchange chromatography (Isolute SCX-2, load in DCM, elution with NH₃ 1N in MeOH), and freeze dried with HCl (0.1N, aq.) to obtain Compound 188 (265 mg, 28%) as an orange solid.

M/Z (M+H)⁺: 278.1

### Example 159: methyl 2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)propanoate

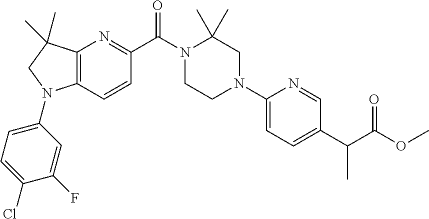 [SMILES: COC(=O)C(C)c1ccc(N2CCN(C(=O)c3ccc4c(n3)C(C)(C)CN4c3ccc(Cl)c(F)c3)C(C)(C)C2)nc1]

Example 159 was prepared according to general procedure (XIV) starting from Compound 71 (130 mg) and Compound 188 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 50:50) to obtain Example 159 (154 mg) as a yellow oil.

M/Z (M[³⁵Cl]+H)⁺: 580.4

### Example 160: 2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)propanoic acid

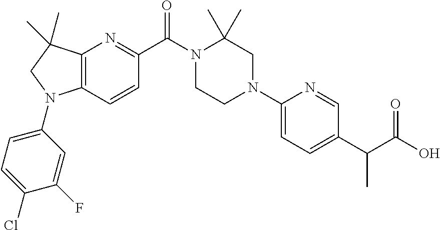 [SMILES: CC(C(=O)O)c1ccc(N2CCN(C(=O)c3ccc4c(n3)C(C)(C)CN4c3ccc(Cl)c(F)c3)C(C)(C)C2)nc1]

Example 160 was prepared according to general procedure (XI) starting from Example 158 (154 mg). The crude was purified by preparative HPLC (Column B, H₂O+0.1% HCOOH/MeCN+0.1% HCOOH 60:40 to 0:100) and freeze dried with HCl (0.1N in water) to obtain Example 160 (98 mg, 39%) as a yellow solid.

¹H-NMR (CD₃OD, 300 MHz) δ: 1.45 (s, 6H, 2CH₃); 1.53 (d, J 7.3 Hz, 3H, CH—CH₃); 1.69 (s, 6H, 2CH₃); 3.77-3.84 (m, 3H, N—CH₂+Ar-CH); 3.88 (s, 2H, N—CH₂); 3.94 (s, 2H, N—CH₂); 4.09 (t, J 5.5 Hz, 2H, N—CH₂); 7.14-7.18 (m, 1H, Ar); 7.22 (dd, J 11.4 2.5 Hz, 1H, Ar); 7.41-7.51 (m, 3H, 3Ar); 7.56 (d, J 8.4 Hz, 1H, Ar); 7.88 (d, J 2.0 Hz, 1H, Ar); 8.12 (dd, J 9.9 1.9 Hz, 1H, Ar).

M/Z (M[³⁵Cl]+H)⁺: 566.4.

### Compound 189: 2-(3-chloropyridin-2-yl)-2-methylpropanenitrile

Compound 189 (4.13 g, 90%) was obtained without further purification as a yellow oil, from 3-chloro-2-fluoropyridine (3.3 g) and isobutyronitrile, according to general procedure (I).

M/Z (M[³⁵Cl]+H⁺): 181.4

### Compound 190: 2-(3-chloropyridin-2-yl)-2-methylpropan-1-amine

Compound 190 (2.07 g, 49%) was obtained as a yellow oil following general procedure (XVa), starting from Compound 189 (4.10 g).

M/Z (M[³⁵Cl]+H⁺): 185.2

### Compound 191: 4-((2-(3-chloropyridin-2-yl)-2-methylpropyl)amino)cyclohexane-1-carbonitrile

To a solution of Compound 190 (100 mg) in THE (5 mL), was added a solution of 4-Oxo-cyclohexanecarbonitrile (100 mg) in THE (5 mL), Acetic acid (31 μL) and Sodium triacetoxyborohydride (230 mg). The reaction mixture was stirred at 25° C. for 18 hours, then quenched with NaHCO₃ (sat. aq. 50 mL), and extracted with EtOAc (3*80 mL). The combined organic layers were washed with brine, dried over MgSO₄ and concentrated under reduced pressure. The crude was purified by flash chromatography (KPNH, CyHex 100% to CyHex/EtOAc 75:25) to obtain Compound 191 (89 mg, 56%) as a clear oil.

M/Z (M[³⁵Cl]+H⁺): 292.2

### Compound 192: 4-(3,3-dimethyl-2,3-dihydro-1H-pyrrolo[3,2-b]pyridin-1-yl)cyclohexane-1-carbonitrile

Compound 192 was prepared according to general procedure (Xd) starting from Compound 191 (158 mg). The crude was purified by flash chromatography (Interchim® 50 μm, CyHex 100% to CyHex/EtOAc 40:60) to obtain Compound 192 (75 mg, 54%) as a yellow oil.

M/Z (M+H)⁺: 256.2.

### Compound 193: 4-(5-bromo-3,3-dimethyl-2,3-dihydro-1H-pyrrolo[3,2-b]pyridin-1-yl)cyclohexane-1-carbonitrile

Compound 193 (395 mg, 96%) was obtained without further purification as a beige solid following general procedure (XVI) in MeCN, starting from Compound 192 (316 mg).

M/Z (M[³⁵Cl][⁸¹Br]+H)⁺: 334.2

### Example 161: methyl 6-(4-(1-(4-cyanocyclohexyl)-3,3-dimethyl-2,3-dihydro-1H-pyrrolo[3,2-b]pyridine-5-carbonyl)-3,3-dimethylpiperazin-1-yl)-2,4-dimethylnicotinate

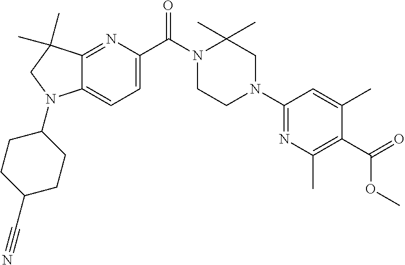 [SMILES: COC(=O)c1c(C)cc(N2CCN(C(=O)c3ccc4c(n3)C(C)(C)CN4C3CCC(C#N)CC3)C(C)(C)C2)nc1C]

Example 161 was prepared according to general procedure (XIV) starting from Compound 193 (200 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 50:50) to obtain Example 161 (125 mg) as a clear oil.

M/Z (M+H)⁺: 559.5

### Example 162: 6-(4-(1-(4-carbamoylcyclohexyl)-3,3-dimethyl-2,3-dihydro-1H-pyrrolo[3,2-b]pyridine-5-carbonyl)-3,3-dimethylpiperazin-1-yl)-2,4-dimethylnicotinic acid

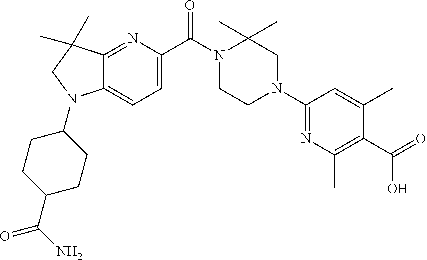 [SMILES: Cc1cc(N2CCN(C(=O)c3ccc4c(n3)C(C)(C)CN4C3CCC(C(N)=O)CC3)C(C)(C)C2)nc(C)c1C(=O)O]

Example 162 was prepared according to general procedure (XI) starting from Example 161 (58 mg). The crude was purified by preparative HPLC (Column B, H₂O+0.1% HCOOH/MeCN+0.1% HCOOH 95:05 to 0:100) and freeze dried with HCl (0.1N in water) to obtain Example 162 (8 mg, 2% over 2 steps) as a yellow solid.

¹H-NMR (DMSO-d₆, 300 MHz) δ: 1.66 (s, 6H, 2CH₃); 1.36-1.57 (m, 10H, 2CH₂+2CH₃); 1.73-1.85 (m, 4H, 2CH₂); 2.02-2.11 (m, 1H, CH); 2.34 (s, 3H, CH₃); 3.26-3.29 (m, 2H, N—CH₂); 3.42-3.51 (m, 1H, N—CH); 3.67 (bs, 2H, N—CH₂); 3.92-3.94 (m, 4H, 2N—CH₂); 6.72-6.78 (m, 2H, NH₂); 7.84 (d, J 8.4 Hz, 1H, Ar); 7.25 (bs, 1H, Ar); 7.31 (d, J 8.4 Hz, 1H, Ar); COOH was not observed.

M/Z (M+H)⁺: 563.5

### Compound 194: N-(2-(3-chloropyridin-2-yl)-2-methylpropyl)-4,4-difluorocyclohexan-1-amine

To a solution of Compound 190 (200 mg) in THE (8 mL), was added a solution of 4,4-difluorocyclohexan-1-one (218 mg), Acetic acid (62 μL) and Sodium triacetoxyborohydride (460 mg). The reaction mixture was stirred at 25° C. for 18 hours, then quenched with NaHCO₃ (sat. aq. 50 mL), and extracted with EtOAc (3*80 mL). The combined organic layers were washed with brine, dried over MgSO₄ and concentrated under reduced pressure to obtain crude Compound 194 (344 mg) as a clear oil.

M/Z (M[³⁵Cl]+H⁺): 303.3

### Compound 195: 1-(4,4-difluorocyclohexyl)-3,3-dimethyl-2,3-dihydro-1H-pyrrolo[3,2-b]pyridine

Compound 195 was prepared according to general procedure (Xd) starting from Compound 194 (328 mg). The crude was purified by flash chromatography (Interchim® 50 μm, CyHex 100% to CyHex/EtOAc 60:40) to obtain Compound 195 (198 mg, 69% over 2 steps) as a beige solid.

M/Z (M+H)⁺: 267.2.

### Compound 196: 5-bromo-1-(4,4-difluorocyclohexyl)-3,3-dimethyl-2,3-dihydro-1H-pyrrolo[3,2-b]pyridine

Compound 196 (230 mg, 90%) was obtained without further purification as a beige solid following general procedure (XVI) in MeCN, starting from Compound 195 (198 mg).

M/Z (M[⁸¹Br]+H)⁺: 345.4.

### Compound 197: methyl 1-(4,4-difluorocyclohexyl)-3,3-dimethyl-2,3-dihydro-1H-pyrrolo[3,2-b]pyridine-5-carboxylate

Compound 197 was prepared according to general procedure (XIX) starting from Compound 196 (230 mg). The crude was purified by flash chromatography (Interchim® 50 μm, CyHex 100% to CyHex/EtOAc 50:50) to obtain Compound 197 (144 mg, 67%) as an orange solid.

M/Z (M+H)⁺: 325.3.

### Compound 198: 1-(4,4-difluorocyclohexyl)-3,3-dimethyl-2,3-dihydro-1H-pyrrolo[3,2-b]pyridine-5-carboxylic acid

Compound 198 was prepared according to general procedure (VII) starting from Compound 197 (144 mg). The crude Compound 198 (118 mg, 85%) was obtained as a white solid.

M/Z (M+H)⁺: 311.3

### Example 163: methyl 2-(6-(4-(1-(4,4-difluorocyclohexyl)-3,3-dimethyl-2,3-dihydro-1H-pyrrolo[3,2-b]pyridine-5-carbonyl)-3,3-dimethylpiperazin-1-yl)pyridin-3-yl)acetate

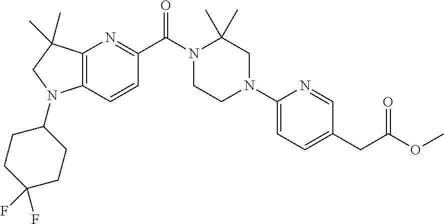 [SMILES: COC(=O)Cc1ccc(N2CCN(C(=O)c3ccc4c(n3)C(C)(C)CN4C3CCC(F)(F)CC3)C(C)(C)C2)nc1]

Example 163 was prepared according to general procedure (VIIIb) starting from Compound 198 (118 mg), Compound 10 (1.1 eq.) and N,N-diisopropylethylamine (3.0 eq.) in MeTHF. The crude was purified by flash chromatography (Interchim® 50 μm, CyHex 100% to CyHex/EtOAc 50:50) to obtain Example 163 (159 mg, 75%) as a yellow solid.

¹H-NMR (DMSO-d₆, 300 MHz) δ: 1.25 (s, 6H, 2CH₃); 1.47 (s, 6H, 2CH₃); 1.61-1.80 (m, 4H, 2CH₂); 1.91-2.11 (m, 4H, 2CH₂); 3.27 (s, 2H, Ar—CH₂); 3.48-3.53 (m, 4H, 2N—CH₂); 3.60 (s, 3H, O—CH₃); 3.67-3.75 (m, 1H, N—CH); 3.78-3.86 (m, 4H, 2N—CH₂); 6.58 (d, J 8.7 Hz, 1H, Ar); 6.83 (d, J 8.4 Hz, 1H, Ar); 7.28 (d, J 8.4 Hz, 1H, Ar); 7.43 (dd, J 2.4, 8.7 Hz, 1H, Ar); 7.95 (d, J 2.4 Hz, 1H, Ar);

M/Z (M+H)⁺: 556.5

### Example 164: 2-(6-(4-(1-(4,4-difluorocyclohexyl)-3,3-dimethyl-2,3-dihydro-1H-pyrrolo[3,2-b]pyridine-5-carbonyl)-3,3-dimethylpiperazin-1-yl)pyridin-3-yl)acetic acid

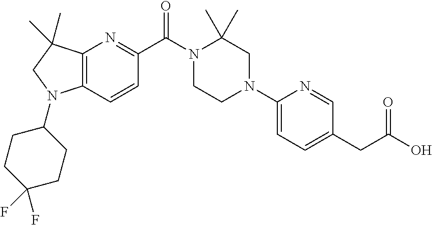 [SMILES: CC1(C)CN(C2CCC(F)(F)CC2)c2ccc(C(=O)N3CCN(c4ccc(CC(=O)O)cn4)CC3(C)C)nc21]

Example 164 was prepared according to general procedure (VII) starting from Example 163 (58 mg). The crude was purified by preparative HPLC (Column B, H₂O+0.1% HCOOH/MeCN+0.1% HCOOH 95:05 to 0:100) and freeze dried with HCl (0.1N in water) to obtain Example 164 (8 mg, 2% over 2 steps) as a yellow solid.

¹H-NMR (DMSO-d₆, 300 MHz) δ: 1.25 (s, 6H, 2CH₃); 1.48 (s, 6H, 2CH₃); 1.61-1.71 (m, 2H, CH₂); 1.76-1.80 (m, 2H, CH₂); 1.91-2.10 (m, 4H, 2CH₂); 2.28 (s, 2H, Ar—CH₂); 3.48-3.56 (m, 4H, 2N—CH₂); 3.67-3.75 (m, 1H, N—CH); 3.84-3.89 (m, 4H, 2N—CH₂); 6.75-6.85 (m, 2H, 2Ar); 7.30 (d, J 8.2 Hz, 1H, Ar); 7.57 (bs, 1H, Ar); 7.93-7.94 (m, 1H, Ar); 12.39 (bs, 1H, COOH);

M/Z (M+H)⁺: 542.4.

### Example 165: 4-(5-(4-(4,6-dimethylpyridin-2-yl)-2,2-dimethylpiperazine-1-carbonyl)-3,3-dimethyl-2,3-dihydro-1H-pyrrolo[3,2-b]pyridin-1-yl)cyclohexane-1-carbonitrile

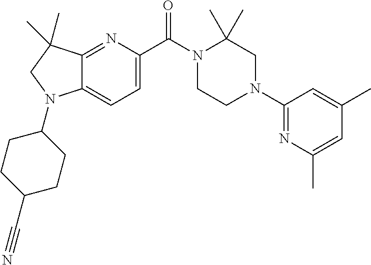 [SMILES: Cc1cc(C)nc(N2CCN(C(=O)c3ccc4c(n3)C(C)(C)CN4C3CCC(C#N)CC3)C(C)(C)C2)c1]

To a solution of Example 161 (58 mg), in Pyridine (1 mL) was added Lithium Iodide (42 mg). The reaction mixture was sealed and heated at 120° C. for 48 hours then concentrated under reduced pressure. The crude was purified by preparative HPLC (Column A, H₂O+0.1% HCOOH/MeCN+0.1% HCOOH 80:20 to 40:60) and freeze dried with HCl water to obtain Example 165 (5 mg, 2% over 2 steps) as a white solid.

¹H-NMR (DMSO-d₆, 300 MHz) δ: 1.24-1.26 (m, 6H, 2CH₃); 1.49 (s, 6H, 2CH₃); 1.58-1.80 (m, 6H, 3CH₂); 1.94-2.34 (m, 8H, CH₂+2CH₃); 3.1-3.24 (m, 1H, CH); 3.31 (s, 2H, CH₂); 3.54-3.56 (m, 3H, N—CH+N—CH₂); 3.83-3.84 (m, 4H, 2N—CH₂); 6.27-6.57 (m, 2H, 2Ar); 6.81 (d, J 8.1 Hz, 1H, Ar); 7.29 (d, J 8.1 Hz, 1H, Ar).

M/Z (M+H)⁺: 501.4.

### Compound 199: N-(2-(3-chloropyridin-2-yl)-2-methylpropyl)cyclopentanamine

To a solution of Compound 190 (150 mg) in THE (8 mL), was added a solution of cyclopentanone (102 mg), Acetic acid (47 μL) and Sodium triacetoxyborohydride (344 mg). The reaction mixture was stirred at 25° C. for 18 hours, then quenched with NaHCO₃ (sat. aq. 50 mL), and extracted with EtOAc (3*80 mL). The combined organic layers were washed with brine, dried over MgSO₄ and concentrated under reduced pressure to obtain crude Compound 199 (167 mg) as a clear oil.

M/Z (M[³⁵Cl]+H⁺): 253.1

### Compound 200: 1-cyclopentyl-3,3-dimethyl-2,3-dihydro-1H-pyrrolo[3,2-b]pyridine

Compound 200 was prepared according to general procedure (Xd) starting from Compound 199 (220 mg). The crude was purified by flash chromatography (Interchim® 50 μm, CyHex 100% to CyHex/EtOAc 55:45) to obtain Compound 200 (114 mg, 39% over 2 steps) as a beige solid.

M/Z (M+H)⁺: 217.2.

### Compound 201: 5-bromo-1-cyclopentyl-3,3-dimethyl-2,3-dihydro-1H-pyrrolo[3,2-b]pyridine

Compound 201 (138 mg, 89%) was obtained without further purification as a brown solid following general procedure (XVI) in MeCN, starting from Compound 200 (114 mg).

M/Z (M[⁸¹Br]+H)⁺: 295.2.

### Compound 202: methyl 1-cyclopentyl-3,3-dimethyl-2,3-dihydro-1H-pyrrolo[3,2-b]pyridine-5-carboxylate

Compound 202 was prepared according to general procedure (XIX) starting from Compound 201 (138 mg). The crude was purified by flash chromatography (Interchim® 50 μm, CyHex 100% to CyHex/EtOAc 60:40) to obtain Compound 202 (88 mg, 69%) as a yellow oil.

M/Z (M+H)⁺: 275.1.

### Compound 203: 1-cyclopentyl-3,3-dimethyl-2,3-dihydro-1H-pyrrolo[3,2-b]pyridine-5-carboxylic acid

Compound 203 was prepared according to general procedure (VII) starting from Compound 202 (88 mg). The crude Compound 203 (68 mg, 81%) was obtained as a beige solid.

M/Z (M[³⁵Cl]+H)⁺: 261.1

### Example 166: methyl 2-(6-(4-(1-cyclopentyl-3,3-dimethyl-2,3-dihydro-1H-pyrrolo[3,2-b]pyridine-5-carbonyl)-3,3-dimethylpiperazin-1-yl)pyridin-3-yl)acetate

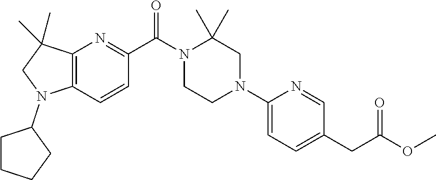 [SMILES: COC(=O)Cc1ccc(N2CCN(C(=O)c3ccc4c(n3)C(C)(C)CN4C3CCCC3)C(C)(C)C2)nc1]

Example 166 was prepared according to general procedure (VIIIb) starting from Compound 203 (68 mg), Compound 111 (1.1 eq.) and N,N-diisopropylethylamine (3.0 eq.) in MeTHF. The crude was purified by flash chromatography (Interchim® 50 μm, CyHex 100% to CyHex/EtOAc 40:60) to obtain Example 166 (92 mg, 70%) as a yellow solid.

¹H-NMR (DMSO-d₆, 300 MHz) δ: 1.25 (s, 6H, 2CH₃); 1.47 (s, 6H, 2CH₃); 1.58-1.68 (m, 6H, 3CH₂); 1.78-1.87 (m, 2H, CH₂); 3.27 (s, 2H, Ar—CH₂); 3.42-3.55 (m, 4H, 2N—CH₂); 3.53 (s, 3H, O—CH₃); 3.78-3.85 (m, 5H, N—CH+2N—CH₂); 6.58 (d, J 8.7 Hz, 1H, Ar); 6.79 (d, J 8.4 Hz, 1H, Ar); 7.25 (d, J 8.4 Hz, 1H, Ar); 7.43 (dd, J 2.4, 8.7 Hz, 1H, Ar); 7.95 (d, J 2.4 Hz, 1H, Ar);

M/Z (M+H)⁺: 506.5

### Example 167: 2-(6-(4-(1-cyclopentyl-3,3-dimethyl-2,3-dihydro-1H-pyrrolo[3,2-b]pyridine-5-carbonyl)-3,3-dimethylpiperazin-1-yl)pyridin-3-yl)acetic acid

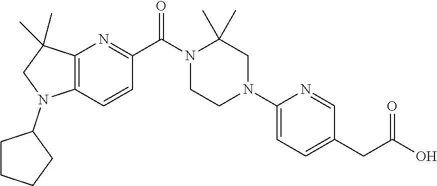 [SMILES: CC1(C)CN(C2CCCC2)c2ccc(C(=O)N3CCN(c4ccc(CC(=O)O)cn4)CC3(C)C)nc21]

Example 167 was prepared according to general procedure (VII) starting from Example 166 (97 mg). The crude was purified by preparative HPLC (Column A, H₂O+0.1% HCOOH/MeCN+0.1% HCOOH 80:20 to 40:60) and freeze dried with HCl (0.1N in water) to obtain Example 167 (44 mg, 43%) as a yellow solid.

¹H-NMR (DMSO-d₆, 300 MHz) δ: 1.25 (s, 6H, 2CH₃); 1.48 (s, 6H, 2CH₃); 1.61-1.71 (m, 2H, CH₂); 1.76-1.80 (m, 2H, CH₂); 1.91-2.10 (m, 4H, 2CH₂); 2.28 (s, 2H, Ar—CH₂); 3.48-3.56 (m, 4H, 2N—CH₂); 3.67-3.75 (m, 1H, N—CH); 3.84-3.89 (m, 4H, 2N—CH₂); 6.75-6.85 (m, 2H, 2Ar); 7.30 (d, J 8.2 Hz, 1H, Ar); 7.57 (bs, 1H, Ar); 7.93-7.94 (m, 1H, Ar); 12.39 (bs, 1H, COOH);

M/Z (M+H)⁺: 542.4.

### Compound 204: methyl 1-(4-cyanocyclohexyl)-3,3-dimethyl-2,3-dihydro-1H-pyrrolo[3,2-b]pyridine-5-carboxylate

Compound 204 was prepared according to general procedure (XIX) starting from Compound 193 (100 mg). The crude was purified by flash chromatography (Interchim® 50 μm, CyHex 100% to CyHex/EtOAc 50:50) to obtain Compound 204 (68 mg, 73%) as a beige solid.

M/Z (M+H)⁺: 314.3.

### Compound 205: methyl 1-(4-cyanocyclohexyl)-3,3-dimethyl-2,3-dihydro-1H-pyrrolo[3,2-b]pyridine-5-carboxylate

Compound 205 was prepared according to general procedure (VII) starting from Compound 204 (117 mg). The crude Compound 205 (92 mg, 82%) was obtained as a white solid.

M/Z (M+H)⁺: 300.2

### Example 168: methyl 2-(6-(4-(1-(4-cyanocyclohexyl)-3,3-dimethyl-2,3-dihydro-1H-pyrrolo[3,2-b]pyridine-5-carbonyl)-3,3-dimethylpiperazin-1-yl)pyridin-3-yl)acetate

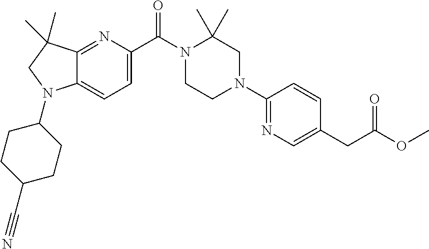 [SMILES: COC(=O)Cc1ccc(N2CCN(C(=O)c3ccc4c(n3)C(C)(C)CN4C3CCC(C#N)CC3)C(C)(C)C2)nc1]

Example 168 was prepared according to general procedure (VIIIb) starting from Compound 205 (92 mg), Compound 111 (1.1 eq.) and N,N-diisopropylethylamine (4.0 eq.) in MeTHF. The crude was purified by flash chromatography (Interchim® 50 μm, CyHex 100% to CyHex/EtOAc 25:75) to obtain Example 168 (143 mg, 85%) as a yellow solid.

¹H-NMR (DMSO-d₆, 300 MHz) δ: 1.24-1.26 (m, 6H, 2CH₃); 1.47-1.53 (m, 6H, 2CH₃); 1.53-1.77 (m, 6H, 3CH₂); 1.95-2.13 (m, 2H, CH₂); 3.18-3.31 (m, 3H, 3CH); 3.49-3.55 (m, 5H, N—CH₂+CH₂C(O)+N—CH); 3.60 (s, 3H, O—CH₃); 3.82-3.87 (m, 4H, 2N—CH₂); 6.59 (d, J 8.7 Hz, 1H, Ar); 6.80 (d, J 8.4 Hz, 1H, Ar); 7.27 (d, J 8.4 Hz, 1H, Ar); 7.44 (dd, J 2.1, 8.7 Hz, 1H, Ar); 7.95 (d, J 2.1 Hz, 1H, Ar);

M/Z (M+H)⁺: 545.5

### Example 169: 2-(6-(4-(1-(4-cyanocyclohexyl)-3,3-dimethyl-2,3-dihydro-1H-pyrrolo[3,2-b]pyridine-5-carbonyl)-3,3-dimethylpiperazin-1-yl)pyridin-3-yl)acetic acid

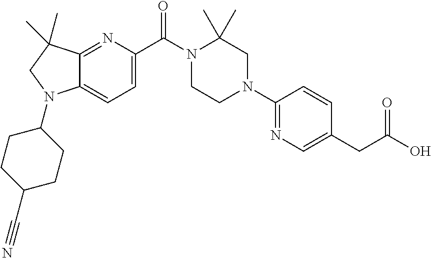 [SMILES: CC1(C)CN(C2CCC(C#N)CC2)c2ccc(C(=O)N3CCN(c4ccc(CC(=O)O)cn4)CC3(C)C)nc21]

Example 169 was prepared according to general procedure (VII) starting from Example 168 (143 mg). The crude was purified by preparative HPLC (Column A, H₂O+0.1% HCOOH/MeCN+0.1% HCOOH 90:10 to 50:50) and freeze dried with HCl (0.1N in water) to obtain Example 169 (17 mg, 12%) as a yellow solid.

¹H-NMR (DMSO-d₆, 300 MHz) δ: 1.24-1.26 (m, 6H, 2CH₃); 1.47-1.53 (m, 7H, CHaHb+2CH₃); 1.58-1.77 (m, 6H, 3 CH₂); 1.95-2.13 (m, 2H, CH₂); 3.20-3.24 (m, 1H, CHaHb); 3.30 (m, 1H, CH); 3.45 (s, 2H, N—CH₂); 3.53-3.54 (m, 3H, CH-N+N—CH₂); 3.82-3.87 (m, 4H, 2N—CH₂); 6.68-6.72 (m, 1H, Ar); 6.79-6.82 (m, 1H, Ar); 7.25-7.29 (m, 1H, Ar); 7.50 (bs, 1H, Ar); 7.93-7.95 (m, 1H, Ar); 12.33 (m, 1H, COOH).

M/Z (M+H)⁺: 531.4.

### Compound 206: 3-chloro-N-(3,4-difluorophenyl)-N-(2-methylallyl)pyrazin-2-amine

Crude Compound 206 (970 mg) was obtained as a brown oil according to general procedure (XVII) on a 500 mg scale, using 4-chloro-3-fluoroaniline.

M/Z (M[³⁵Cl]+H)⁺: 296.2.

### Compound 207: 5-(3,4-difluorophenyl)-7,7-dimethyl-6,7-dihydro-5H-pyrrolo[2,3-b]pyrazine

Compound 207 was prepared according to general procedure (XVIII) starting from crude Compound 206 (970 mg). The crude was purified by flash chromatography (Interchim® 50 μm, CyHex 100% to CyHex/EtOAc 60:40) to obtain Compound 207 (482 mg, 55% over 2 steps) as a yellow solid.

M/Z (M+H)⁺: 262.2

### Compound 208: 2-bromo-5-(3,4-difluorophenyl)-7,7-dimethyl-6,7-dihydro-5H-pyrrolo[2,3-b]pyrazine

Compound 208 (476 mg, 76%) was obtained as a brown solid following general procedure (XVI) in MeCN without further purification, starting from Compound 207 (482 mg).

M/Z (M[⁸¹Br]+H)⁺: 340.1.

### Example 170: methyl 2-(6-(4-(5-(3,4-difluorophenyl)-7,7-dimethyl-6,7-dihydro-5H-pyrrolo[2,3-b]pyrazine-2-carbonyl)-3,3-dimethylpiperazin-1-yl)pyridin-3-yl)acetate

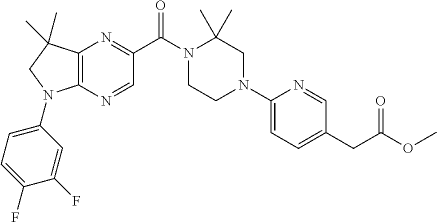 [SMILES: COC(=O)Cc1ccc(N2CCN(C(=O)c3cnc4c(n3)C(C)(C)CN4c3ccc(F)c(F)c3)C(C)(C)C2)nc1]

Example 170 was prepared according to general procedure (XIV) starting from Compound 208 (75 mg) and Compound 111 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 170 (60 mg) as a yellow solid.

¹H-NMR (DMSO-d₆, 300 MHz) δ: 1.39 (s, 6H, 2CH₃); 1.50 (s, 6H, 2CH₃); 3.46-3.53 (m, 4H, N—CH₂+CH₂C(O)); 3.60 (s, 3H, O—CH₃); 3.76-3.90 (m, 4H, 2N—CH₂); 3.98 (s, 2H, N—CH₂); 6.58-6.61 (m, 1H, Ar); 7.39-7.55 (m, 3H, Ar); 7.63- 7.70 (m, 1H, Ar); 7.96 (s, 1H, Ar); 8.06-8.16 (m, 1H, Ar); 8.19 (s, 1H, Ar).

M/Z (M+H)⁺: 551.4.

### Example 171: 2-(6-(4-(5-(3,4-difluorophenyl)-7,7-dimethyl-6,7-dihydro-5H-pyrrolo[2,3-b]pyrazine-2-carbonyl)-3,3-dimethylpiperazin-1-yl)pyridin-3-yl)acetic acid

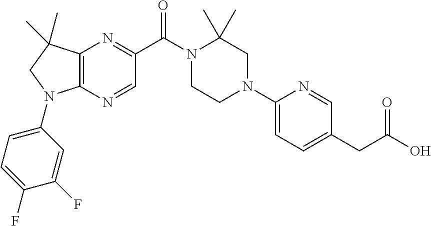 [SMILES: CC1(C)CN(c2ccc(F)c(F)c2)c2ncc(C(=O)N3CCN(c4ccc(CC(=O)O)cn4)CC3(C)C)nc21]

Example 171 was prepared according to general procedure (VII) starting from Example 170 (60 mg). The crude was purified by preparative HPLC (Column B, H₂O+0.1% HCOOH/MeCN+0.1% HCOOH 65:35 to 0:100) and freeze dried with HCl (0.1N in water) to obtain Example 171 (28 mg, 22% over 2 steps) as a white solid.

¹H-NMR (DMSO-d₆, 300 MHz) δ: 1.40 (s, 6H, 2CH₃); 1.56 (s, 6H, 2CH₃); 3.66 (s, 2H, CH₂C(O)); 3.69-3.77 (m, 2H, N—CH₂); 3.87-3.96 (m, 2H, N—CH₂); 3.98 (bs, 4H, 2N—CH₂); 7.26-7.44 (m, 1H, Ar); 7.48-7.55 (m, 1H, Ar); 7.61-7.70 (m, 1H, Ar); 7.91-8.03 (m, 2H, 2Ar); 8.08-8.13 (m, 1H, Ar); 8.23 (s, 1H, Ar); 12.25-14.14 (s, 1H, COOH).

M/Z (M+H)⁺: 537.3.

### Example 172: methyl 2-(6-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)pyridin-3-yl)acetate

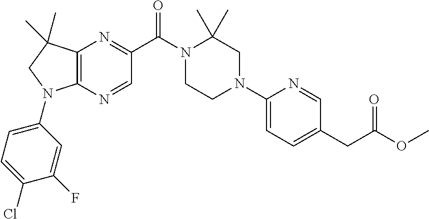 [SMILES: COC(=O)Cc1ccc(N2CCN(C(=O)c3cnc4c(n3)C(C)(C)CN4c3ccc(Cl)c(F)c3)C(C)(C)C2)nc1]

Example 172 was prepared according to general procedure (XIV) starting from Compound 128 (1.0 g) and Compound 111 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 50:50) to obtain Example 172 (0.82 g, 51%) as a yellow solid.

M/Z (M[³⁵Cl]+H)⁺: 567.3

### Example 173: 2-(6-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)pyridin-3-yl)acetic acid

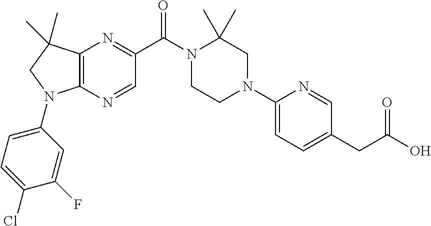 [SMILES: CC1(C)CN(c2ccc(Cl)c(F)c2)c2ncc(C(=O)N3CCN(c4ccc(CC(=O)O)cn4)CC3(C)C)nc21]

Example 173 was prepared according to general procedure (VII) starting from Example 172 (84 mg). The crude was purified by preparative HPLC (Column B, H₂O+0.1% HCOOH/MeCN+0.1% HCOOH 65:35 to 0:100) and freeze dried with HCl (0.1N in water) to obtain Example 173 (50 mg, 54% over 2 steps) as a yellow solid.

¹H-NMR (DMSO-d₆, 300 MHz) δ: 1.40 (s, 6H, 2CH₃); 1.56 (s, 6H, 2CH₃); 3.64 (s, 2H, CH₂C(O)); 3.66-3.75 (m, 2H, N—CH₂); 3.86-3.95 (m, 2H, N—CH₂); 3.98 (s, 2H, N—CH₂); 3.99 (s, 2H); 7.15-7.43 (m, 1H, Ar); 7.63 (t, J 8.8 Hz, 1H, Ar); 7.73 (dd, J 9.0, 2.6 Hz, 1H, Ar); 7.85-8.00 (m, 2H, 2Ar); 8.10 (dd, J 12.5, 2.6 Hz, 1H, Ar); 8.25 (s, 1H, Ar); 12.08-14.53 (s, 1H, COOH).

M/Z (M[³⁵Cl]+H)⁺: 553.3.

### Compound 209: methyl 2-(6-chloropyridin-3-yl)-2-methylpropanoate

Compound 209 (210 mg, 37%) was obtained without further purification as a yellow oil, from methyl 2-(6-chloropyridin-3-yl)acetate (500 mg), according to general procedure (XIII) in THE with NaH (3.0 eq.) and Mel (3.0 eq).

M/Z (M[³⁵Cl]+H)⁺: 214.1

### Compound 210: tert-butyl 4-(5-(1-methoxy-2-methyl-1-oxopropan-2-yl)pyridin-2-yl)-2,2-dimethylpiperazine-1-carboxylate

Compound 210 was prepared according to general procedure (Xb), starting from Compound 209 (330 mg) and tert-butyl 2,2-dimethylpiperazine-1-carboxylate (1.2 eq.). The crude Compound 210 (580 mg) was obtained as a yellow solid and engaged directly in the following step.

M/Z (M+H)⁺: 392.3

### Compound 211: methyl 2-(6-(3,3-dimethylpiperazin-1-yl)pyridin-3-yl)-2-methylpropanoate hydrochloride

Compound 211 was prepared according to general procedure (XII) starting from Compound 210 (580 mg) in DCM using TFA. The reaction mixture was concentrated under reduced pressure then purified by Ion Exchange chromatography (Isolute SCX-2, load in DCM, elution with NH₃ 1N in MeOH), and freeze dried with HCl (0.1N, aq.) to obtain Compound 211 (210 mg, 42% over 2 steps) as an orange solid.

M/Z (M+H)⁺: 292.3

### Example 174: methyl 2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)-2-methylpropanoate

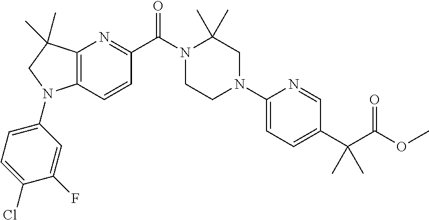 [SMILES: COC(=O)C(C)(C)c1ccc(N2CCN(C(=O)c3ccc4c(n3)C(C)(C)CN4c3ccc(Cl)c(F)c3)C(C)(C)C2)nc1]

Example 174 was prepared according to general procedure (XIV) starting from Compound 71 (100 mg) and Compound 211 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 50:50) to obtain Example 174 (120 mg, 63%) as a yellow oil.

¹H-NMR (DMSO-d₆, 300 MHz) δ: 1.35 (s, 6H, 2CH₃); 1.48 (s, 6H, 2CH₃); 1.50 (s, 6H, 2CH₃); 3.48-3.54 (m, 2H, N—CH₂); 3.58 (s, 3H, O—CH₃); 3.78-3.86 (m, 6H, 3N—CH₂); 6.58-6.61 (m, 1H, Ar); 7.17-7.21 (m, 1H, Ar); 7.30-7.37 (m, 2H, Ar); 7.47-7.58 (m, 3H, 3Ar); 8.05 (d, J 8.6 Hz, 1H, Ar).

M/Z (M[³⁵Cl]+H)⁺: 594.4

### Example 175: 2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)-2-methylpropanoic acid

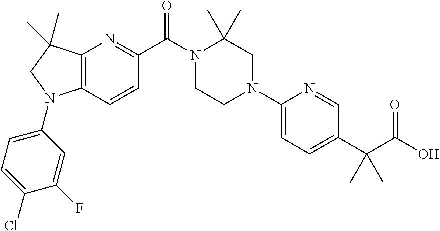 [SMILES: CC1(C)CN(c2ccc(Cl)c(F)c2)c2ccc(C(=O)N3CCN(c4ccc(C(C)(C)C(=O)O)cn4)CC3(C)C)nc21]

Example 175 was prepared according to general procedure (XI) starting from Example 174 (154 mg). The crude was purified by preparative HPLC (Column B, H₂O+0.1% HCOOH/MeCN+0.1% HCOOH 60:40 to 0:100) and freeze dried with HCl (0.1N in water) to obtain Example 175 (98 mg, 39%) as a yellow solid.

¹H-NMR (DMSO-d₆, 300 MHz) δ: 1.36 (s, 6H, 2CH₃); 1.51 (s, 6H, 2CH₃); 1.56 (s, 6H, 2CH₃); 3.70-3.76 (m, 2H, N—CH₂); 3.87 (bs, 2H, N—CH₂); 3.93-3.99 (m, 4H, 2N—CH₂); 7.19 (dd, J 8.6 2.9 Hz, 1H, Ar); 7.31-7.36 (m, 2H, 2Ar); 7.40 (d, J 8.6 Hz, 1H, Ar); 7.53-7.59 (m, 2H, 2Ar); 7.75-7.79 (m, 1H, Ar); 8.01-8.08 (m, 1H, Ar); COOH was not observed.

M/Z (M[³⁵Cl]+H)⁺: 580.4

### Compound 212: 4-methyl-N-(pyridin-3-yl)benzenesulfonamide

To a solution of 3-aminopyridine (10.0 g) in 60 ml of pyridine, was added p-tosyl chloride (24.3 g) and the solution was heated at 120° C. After 2 hours the solution was poured in cold water (100 mL, 0° C.), the white precipitate was collected by filtration and washed with water. The solid was dissolved in THF, washed with brine, and dried over MgSO₄ to afford Compound 212 (20.0 g, 75%) as a white solid.

M/Z (M+H)⁺: 249.2

### Compound 213: 4-methyl-N-(3-methylbut-3-en-1-yl)-N-(pyridin-3-yl)benzenesulfonamide

To a solution of Compound 212 (5.00 g) in THE (150 mL) was added 3-methylbut-3-en-1-ol (1.73 g) and Triphenylphosphine (5.28 g). Then, Diisopropyl azodicarboxylate (407 mg) was added dropwise at 0° C. The reaction was stirred at 25° C. for 4 hours. The reaction was quenched with water (150 mL), and then extracted with EtOAc (3*200 mL). The combined organic layers were washed with brine (300 mL) and dried over Na₂SO₄. The crude was purified by flash chromatography (Petroleum Ether/EtOAc, 80:20) to obtain Compound 213 (5.00 g, 78%) as a white solid.

M/Z (M+H)⁺: 317.2

### Compound 214: 4,4-dimethyl-1-tosyl-1,2,3,4-tetrahydro-1,5-naphthyridine

To a solution of Compound 213 (5.00 g) in EtOH (100 mL) was added Iron tri(4-methoxypent-3-en-2-one) (5.58 g), 2-(tert-butylperoxy)-2-methylpropane (2.31 g) and silylbenzene (4.28 g). The reaction was heated at 60° C. for 16 hours then concentrated under reduced pressure. The residue was quenched with water (100 mL), and then extracted with EtOAc (3*200 mL). The combined organic layers were washed with brine (200 mL) and dried over Na₂SO₄. The crude was purified by flash chromatography (Petroleum Ether/EtOAc, 75:25) to obtain Compound 214 (1.00 g, 20%) as a yellow solid.

¹H NMR (CDCl₃, 400 MHz,) δ: 1.08-1.10 (m, 6H, C(CH₃)₂); 1.40-1.46 (m, 2H, CH₂); 2.38 (s, 3H, ArCH₃); 3.78-3.84 (m, 2H, N—CH₂); 7.10-7.15 (m, 1H); 7.22 (d, J 7.4 Hz, 2H); 7.45 (d, J 7.4 Hz, 2H); 8.16 (d, J 8.4 Hz, 1H); 8.35-8.40 (m, 1H).

M/Z (M+H)⁺: 317.2

### Compound 215: 4,4-dimethyl-1,2,3,4-tetrahydro-1,5-naphthyridine

A solution of Compound 214 (1.50 g) in HBr (aq. 40% wt, 50 mL) was heated at 70° C. for 12 hours. The solution was treated with NaHCO₃ (1M aq., to pH=9) and extracted with EtOAc (3*100 mL). The combined organic layers were washed with brine (200 mL) and dried over Na₂SO₄. The crude was purified by flash chromatography (Petroleum Ether/EtOAc, 75:25) to obtain Compound 215 (600 mg, 78%) as a yellow solid.

M/Z (M+H)⁺: 163.2

### Compound 216: 1-(4-chloro-3-fluorophenyl)-4,4-dimethyl-1,2,3,4-tetrahydro-1,5-naphthyridine

To a solution of Compound 215 (160 mg) in Toluene (15 mL) was added 1,1′-Bis(diphenylphosphino)ferrocene-palladium(II) dichloride (72 mg), 1-chloro-2-fluoro-4-iodobenzene (379 mg), 2-(Dicyclohexylphosphanyl)-2′,4′,6′-tris(isopropyl)biphenyl (47 mg) and sodium 2-methylpropan-2-olate (190 mg). The reaction was heated at 100° C. for 5 hours. The reaction was quenched with water (30 mL), and then extracted with EtOAc (3*50 mL). The combined organic layers were washed with brine (200 mL) and dried over Na₂SO₄. The crude was purified by flash chromatography (Petroleum Ether/EtOAc, 75:25) to obtain Compound 216 (115 mg, 40%) as a yellow solid.

M/Z (M[³⁵Cl]+H)⁺: 291.2

### Compound 217: 6-bromo-1-(4-chloro-3-fluorophenyl)-4,4-dimethyl-1,2,3,4-tetrahydro-1,5-naphthyridine

To a solution of Compound 216 (115 mg) in MeCN (15 mL) was added acetic acid (25 μL) and N-Bromosuccinimide (70 mg). The reaction was stirred at 25° C. for 2 hours. The solution was treated with NaHCO₃ (1M aq., to pH=9) and extracted with EtOAc (3*50 mL). The combined organic layers were washed with brine (50 mL) and dried over Na₂SO₄. The crude was purified by flash chromatography (Petroleum Ether/EtOAc, 90:10) to obtain Compound 217 (120 mg, 82%) as a yellow solid.

M/Z (M[⁸¹Br][³⁵Cl]+H)⁺: 369.0

¹H NMR (DMSO-d₆, 400 MHz) δ 1.26-1.30 (m, 6H, C(CH₃)₂); 1.84-1.89 (m, 2H, CH₂); 3.59-3.63 (m, 2H, N—CH₂); 7.05 (d, J 8.6 Hz, 1H, Ar); 7.13 (ddd, J 4.2, 3.5, 2.9 Hz, 2H, 2Ar); 7.37 (dd, J=11.3, 2.5 Hz, 1H, Ar); 7.56 (t, J=8.7 Hz, 1H, Ar).

### Example 176: methyl 2-(6-(4-(5-(4-chloro-3-fluorophenyl)-8,8-dimethyl-5,6,7,8-tetrahydro-1,5-naphthyridine-2-carbonyl)-3,3-dimethylpiperazin-1-yl)pyridin-3-yl)acetate

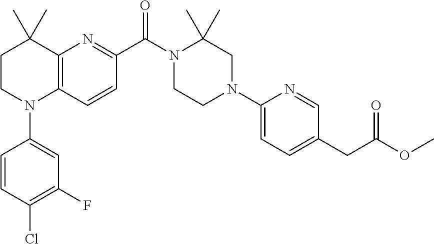 [SMILES: COC(=O)Cc1ccc(N2CCN(C(=O)c3ccc4c(n3)C(C)(C)CCN4c3ccc(Cl)c(F)c3)C(C)(C)C2)nc1]

To a solution of Compound 217 (100 mg) in Dioxane (10 mL) was added Xantphos Pd G4 (26 mg), Triethylamine (60 μL) and Compound 111 (71 mg). The reaction was heated at 100° C. for 5 hours in CO atmosphere. The reaction diluted with water (100 mL), and then extracted with EtOAc (3*50 mL). The organic phase was washed with brine (100 mL) and dried with anhydrous sodium sulfate. The combined organic layers were washed with brine (50 mL) and dried over Na₂SO₄. The crude was purified by flash chromatography to obtain Example 176 (140 mg, 89%) as a yellow solid.

¹H NMR (DMSO-d₆, 400 MHz) δ 1.30-1.36 (m, 6H, C(CH₃)₂); 1.49 (s, 6H, C(CH₃)₂); 1.86-1.89 (m, 2H, CH₂); 3.50-3.56 (m, 4H, CH₂C(O)+N—CH₂); 3.61 (s, 3H, O—CH₃); 3.63-3.69 (m, 2H, N—CH₂); 3.80-3.87 (m, 2H, N—CH₂); 3.92-3.96 (m, 2H, N—CH₂); 6.58-6.61 (m, 1H, Ar); 7.10 (d, J 8.6 Hz, 1H, Ar); 7.16-7.20 (m, 1H, Ar); 7.25 (d, J 8.6 Hz, 1H, Ar); 7.39-7.47 (m, 2H, 2Ar); 7.60 (t, J=8.7 Hz, 1H, Ar); 7.97 (d, J=2.2 Hz, 1H, Ar).

M/Z (M[³⁵Cl]+H)⁺: 580.2

### Example 177: 2-(6-(4-(5-(4-chloro-3-fluorophenyl)-8,8-dimethyl-5,6,7,8-tetrahydro-1,5-naphthyridine-2-carbonyl)-3,3-dimethylpiperazin-1-yl)pyridin-3-yl)acetic acid

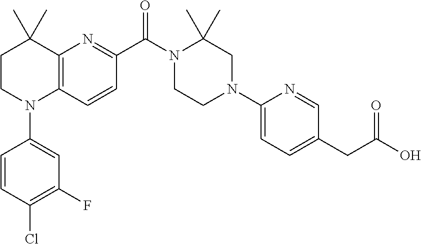 [SMILES: CC1(C)CCN(c2ccc(Cl)c(F)c2)c2ccc(C(=O)N3CCN(c4ccc(CC(=O)O)cn4)CC3(C)C)nc21]

To a solution of Example 176 (150 mg) in THE (2 mL) was added LiOH (124 mg) and H₂O (3 mL). The reaction mixture was stirred at 25° C. for 1 hour, concentrated under reduced pressure and the residue was purified by preparative HPLC (MeCN/H₂O+0.05% NH₃) to obtain Example 177 (33 mg, 22%) as a white solid.

¹HNMR (DMSO-d₆, 400 MHz) δ: 1.34 (s, 6H, C(CH₃)₂); 1.49 (s, 6H, C(CH₃)₂); 1.89-1.97 (m, 2H, CH₂); 3.41 (s, 2H, CH₂C(O)); 3.50-3.56 (m, 2H, N—CH₂); 3.64-3.69 (m, 2H, N—CH₂); 3.84 (s, 2H, N—CH₂); 3.91-3.95 (m, 2H, N—CH₂); 6.58-6.61 (m, 1H, Ar); 7.10 (d, J 8.6 Hz, 1H, Ar); 7.16-7.21 (m, 1H, Ar); 7.25 (d, J 8.6 Hz, 1H, Ar); 7.40-7.46 (m, 2H, 2Ar); 7.60 (t, J=8.7 Hz, 1H, Ar); 7.95 (d, J=2.2 Hz, 1H, Ar). COOH was not observed.

M/Z (M[³⁵Cl]+H)⁺: 566.2

### Compound 218: 2-bromo-7,7-dimethyl-6,7-dihydropyrazolo[1,5-a]pyrimidin-5(4H)-one

To a solution of 3-bromo-1H-pyrazol-5-amine (1.00 g) in 1,4-dioxane (15 mL) was added cesium carbonate (4.02 g) and 3-methylbut-2-enoyl chloride (805 mg). The reaction was subjected to microwave irradiation at 80° C. for 2 hours and 120° C. for 2 hours. The reaction mixture was filtered and the filtrate was concentrated under reduced pressure. The residue was purified by flash chromatography (petroleum ether 100% to petroleum ether/EtOAc 70:30) to obtain Compound 218 (1.1 g) as a yellow solid.

M/Z (M[⁷⁹Br]+H)⁺: 244.0.

### Compound 219: 2-bromo-7,7-dimethyl-4,5,6,7-tetrahydropyrazolo[1,5-a]pyrimidine

Compound 218 (3.40 g) was dissolved in BH₃-THF (1M, 139 mL) at 0° C. The reaction mixture was stirred at 25° C. for 18 hours then MeOH (100 mL) was added and the mixture was refluxed for 1 hour, then concentrated under reduced pressure. The residue was treated with H₂O (100 mL), extracted with EtOAc (3*70 mL), the combined organic layers were washed with brine (50 mL), dried over Na₂SO₄ and concentrated under reduced pressure to obtain Compound 219 (3.26 g) as a white solid.

M/Z (M[⁷⁹Br]+H)⁺: 230.0.

### Compound 220: 2-bromo-4-(4-chloro-3-fluorophenyl)-7,7-dimethyl-4,5,6,7-tetrahydropyrazolo[1,5-a]pyrimidine

To a solution of Compound 219 (1.50 g) in Toluene (100 mL) was added 1-chloro-2-fluoro-4-iodobenzene (2.51 g), Pd(dppf)Cl₂ (477 mg), 2-(Dicyclohexylphosphanyl)-2′,4′,6′-tris(isopropyl)biphenyl (311 mg) and sodium 2-methylpropan-2-olate (1.25 g). The reaction mixture was sparged with Nitrogen and heated at 100° C. for 2 hours. The reaction mixture was filtered and the filtrate concentrated under reduced pressure. The residue was purified by flash chromatography (petroleum ether 100% to petroleum ether/EtOAc 70:30) to obtain Compound 220 (1.24 g, 53%) as a brown solid.

¹H NMR (DMSO-d₆, 400 MHz) δ: 1.48 (s, 6H, C(CH₃)₂); 2.07-2.18 (m, 2H, CH₂); 3.65-3.78 (m, 2H, N—CH₂); 5.75 (s, 1H, Ar); 7.14-7.21 (m, 1H, Ar); 7.29-7.39 (m, 1H, Ar); 7.50-7.54 (m, 1H, Ar).

M/Z (M[⁷⁹Br][³⁵Cl]+H)⁺: 358.0.

### Example 178: methyl 2-(6-(4-(4-(4-chloro-3-fluorophenyl)-7,7-dimethyl-4,5,6,7-tetrahydropyrazolo[1,5-a]pyrimidine-2-carbonyl)-3,3-dimethylpiperazin-1-yl)pyridin-3-yl)acetate

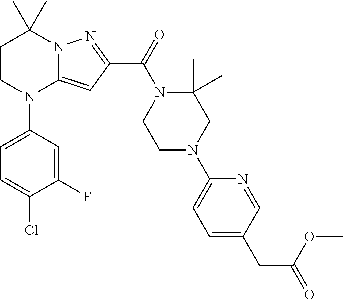 [SMILES: COC(=O)Cc1ccc(N2CCN(C(=O)c3cc4n(n3)C(C)(C)CCN4c3ccc(Cl)c(F)c3)C(C)(C)C2)nc1]

To a solution of Compound 220 (500 mg) in dioxane (50 mL) was added Compound 111 (734 mg), XantPhos Pd G4 (134 mg) and NEt₃ (282 mg). The reaction was heated at 110° C. for 32 hours under CO atmosphere. The reaction mixture was filtered and the filtrate was concentrated under reduced pressure. The residue was purified by flash chromatography (Petroleum ether 100% to petroleum ether/EtOAc 5:95) to obtain Example 178 (189 mg, 23%) as a brown solid.

¹H NMR (DMSO-d₆, 400 MHz) δ: 1.45 (s, 6H, C(CH₃)₂); 1.53 (s, 6H, C(CH₃)₂); 2.12-2.20 (m, 2H, CH₂); 3.47 (s, 2H, CH₂C(O)); 3.50-3.54 (m, 2H, N—CH₂); 3.60 (s, 3H, O—CH₃); 3.70-3.79 (m, 2H, N—CH₂); 3.83 (s, 2H, N—CH₂); 4.10-4.20 (m, 2H, N—CH₂); 5.89 (s, 1H, Ar); 6.55-6.61 (m, 1H, Ar); 7.16-7.25 (m, 1H, Ar); 7.30-7.47 (m, 2H, 2Ar); 7.47-7.59 (m, 1H, Ar); 7.92-7.97 (m, 1H).

M/Z (M[³⁵Cl]+H)⁺: 569.2

### Example 179: 2-(6-(4-(4-(4-chloro-3-fluorophenyl)-7,7-dimethyl-4,5,6,7-tetrahydropyrazolo[1,5-a]pyrimidine-2-carbonyl)-3,3-dimethylpiperazin-1-yl)pyridin-3-yl)acetic acid

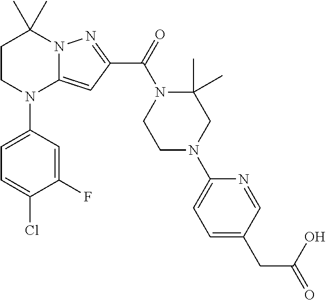 [SMILES: CC1(C)CN(c2ccc(CC(=O)O)cn2)CCN1C(=O)c1cc2n(n1)C(C)(C)CCN2c1ccc(Cl)c(F)c1]

To a solution of Example 178 (189 mg) in THE (10 mL) was added LiOH (39.8 mg) dissolved in water (2.00 mL) at 0° C. The reaction was stirred at 25° C. for 3 hours. The mixture was adjusted pH to 5 with HCl (aq. 1M) and extracted with EtOAc (3*20 mL). The combined organic layer was washed with brine (20 mL), dried over Na₂SO₄ and concentrated under reduced pressure. The crude was purified by Prep-HPLC (MeCN/H₂O with 0.05% HCOOH) to obtain Example 178 (73 mg, 39%) as a white solid.

¹H NMR (DMSO-d₆, 400 MHz) δ: 1.45 (s, 6H, C(CH₃)₂); 1.53 (s, 6H, C(CH₃)₂); 2.13-2.22 (m, 2H, CH₂); 3.41 (s, 2H, CH₂C(O)); 3.45-3.50 (m, 2H, N—CH₂); 3.70-3.77 (m, 2H, N—CH₂); 3.83 (s, 2H, N—CH₂); 4.10-4.19 (m, 2H, N—CH₂); 5.89 (s, 1H, Ar); 6.55-6.61 (m, 1H, Ar); 7.16-7.25 (m, 1H, Ar); 7.30-7.47 (m, 2H, 2Ar); 7.47-7.59 (m, 1H, Ar); 7.92-7.97 (m, 1H), 12.15 (bs, 1H, COOH).

M/Z (M[³⁵Cl]+H)⁺: 555.2.

### Example 180: methyl 6-(4-(5-(3,4-difluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinate

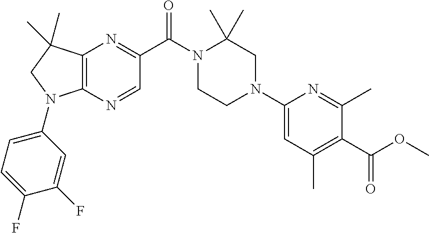 [SMILES: COC(=O)c1c(C)cc(N2CCN(C(=O)c3cnc4c(n3)C(C)(C)CN4c3ccc(F)c(F)c3)C(C)(C)C2)nc1C]

Example 180 was prepared according to general procedure (XIV) starting from Compound 208 (80 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 180 (116 mg) as a yellow solid.

¹H-NMR (DMSO-d₆, 300 MHz) δ: 1.39 (s, 6H, 2CH₃); 1.50 (s, 6H, 2CH₃); 2.23 (s, 3H, ArCH₃); 2.34 (s, 3H, ArCH₃); 3.49-3.53 (m, 2H, 2N—CH₂); 3.68-3.85 (m, 5H, O—CH₃+N—CH₂); 3.90-3.95 (m, 2H, N—CH₂); 3.98 (s, 2H, N—CH₂); 6.37 (s, 1H, Ar); 7.36-7.56 (m, 2H, 2Ar); 7.61-7.69 (m, 1H, Ar); 8.11 (ddd, J 13.8, 7.4, 2.2 Hz, 1H, Ar); 8.19 (s, 1H, Ar);

M/Z (M+H)⁺: 565.1.

### Example 181: 6-(4-(5-(3,4-difluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid

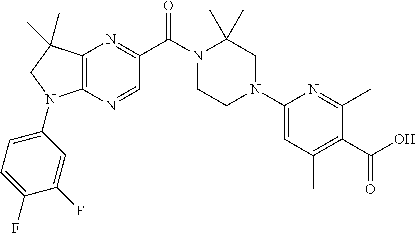 [SMILES: Cc1cc(N2CCN(C(=O)c3cnc4c(n3)C(C)(C)CN4c3ccc(F)c(F)c3)C(C)(C)C2)nc(C)c1C(=O)O]

Example 181 was prepared according to general procedure (XI) starting from Example 180 (116 mg). The crude was purified by preparative HPLC (Column B, H₂O+0.1% HCOOH/MeCN+0.1% HCOOH 65:35 to 0:100) and freeze dried with HCl (0.1N in water) to obtain Example 181 (33 mg, 24% over 2 steps) as a white solid.

¹H-NMR (DMSO-d₆, 300 MHz) δ: 1.38 (s, 6H, 2×CH₃); 1.51 (s, 6H, 2×CH₃); 2.30 (s, 3H, CH₃); 2.45 (s, 3H, CH₃); 3.58-3.65 (m, 2H, N—CH₂); 3.79-3.88 (m, 2H, N—CH₂); 3.92 (s, 2H, N—CH₂); 3.97 (s, 2H, N—CH₂); 6.46-6.73 (bs, 1H, Ar); 7.50 (dd, J 19.2, 9.4 Hz, 1H, Ar); 7.59-7.69 (m, 1H, Ar); 8.11 (ddd, J 13.8, 7.4, 2.2 Hz, 1H, Ar); 8.19 (s, 1H, Ar); COOH was not observed.

M/Z (M+H)⁺: 551.4.

### Compound 221: 3-chloro-N-cyclopentylpyrazin-2-amine

A solution of 2,3-dichloropyrazine (500 mg) and cyclopentanamine (1.63 mL) in MeTHF (5 mL) was heated at 80° C. for 16 hours. The reaction mixture was washed with HCl (aq. 0.1N, 10 mL), with Brine (10 mL), dried over MgSO₄, and concentrated under reduced pressure to obtain crude Compound 221 (560 mg).

M/Z (M[³⁵Cl]+H)⁺: 198.2

### Compound 222: 3-chloro-N-cyclopentyl-N-(2-methylallyl)pyrazin-2-amine

To a solution of Compound 221 (560 mg) in THE (28 mL) was added potassium tert-butoxide (445 mg) at 0° C. After 10 minutes, 3-bromo-2-methylprop-1-ene (590 μL) was added and the reaction mixture was stirred at 25° C. for 17 hours, then heated at 50° C. for 4 hours. Another portion of base (445 mg) and 3-bromo-2-methylprop-1-ene (590 μL) were added and the mixture was heated at 50° C. for another 17 hours. The reaction mixture was quenched with NH₄Cl (sat. aq. 150 mL), diluted with water (50 mL), and extracted with EtOAc (3*150 mL). The combined organic layers were washed with brine (50 mL), dried over MgSO₄, and concentrated under reduced pressure. The residue was purified by flash chromatography (Interchim® 50 μm, CyHex 100% to CyHex/EtOAc 90:10) to obtain Compound 222 (382 mg, 45% over 2 steps) as a yellow oil.

M/Z (M[³⁵Cl]+H)⁺: 252.1

### Compound 223: 5-cyclopentyl-7,7-dimethyl-6,7-dihydro-5H-pyrrolo[2,3-b]pyrazine

Compound 223 was prepared according to general procedure (XVIII) starting from Compound 222 (381 mg). The crude was purified by flash chromatography (Interchim® 50 μm, CyHex 100% to CyHex/EtOAc 50:50) to obtain Compound 223 (239 mg, 73%) as a yellow oil.

M/Z (M[³⁵Cl]+H)⁺: 218.2

### Compound 224: 2-bromo-5-cyclopentyl-7,7-dimethyl-6,7-dihydro-5H-pyrrolo[2,3-b]pyrazine

Compound 224 (320 mg, 98%) was obtained without further purification as a yellow solid following general procedure (XVI) in MeCN, starting from Compound 223 (239 mg).

M/Z (M[³⁵Cl][⁸¹Br]+H)⁺: 298.2

### Example 182: methyl 6-(4-(5-cyclopentyl-7,7-dimethyl-6,7-dihydro-5H-pyrrolo[2,3-b]pyrazine-2-carbonyl)-3,3-dimethylpiperazin-1-yl)-2,4-dimethylnicotinate

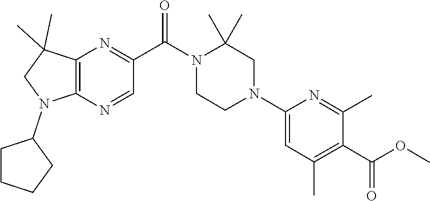 [SMILES: COC(=O)c1c(C)cc(N2CCN(C(=O)c3cnc4c(n3)C(C)(C)CN4C3CCCC3)C(C)(C)C2)nc1C]

Example 182 was prepared according to general procedure (XIV) starting from Compound 224 (100 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 60:40) to obtain Example 182 (90 mg, 51%) as a clear oil.

¹H-NMR (DMSO-d₆, 300 MHz) δ: 1.28 (s, 6H, 2CH₃); 1.46 (s, 6H, 2CH₃); 1.57-1.72 (m, 6H, 3CH₂); 1.79-1.86 (m, 2H, CH₂); 2.23 (s, 3H, CH₃); 2.40 (s, 3H, CH₃); 3.43 (s, 2H, N—CH₂); 3.52-3.55 (m, 2H, N—CH₂); 3.78 (s, 3H, O—CH₃); 3.78-3.90 (m, 4H, 2N—CH₂); 4.39 (q, J 7.3 Hz, 1H, N—CH); 6.36 (s, 1H, Ar); 7.95 (s, 1H, Ar).

M/Z (M+H)⁺: 521.5

### Example 183: 6-(4-(5-cyclopentyl-7,7-dimethyl-6,7-dihydro-5H-pyrrolo[2,3-b]pyrazine-2-carbonyl)-3,3-dimethylpiperazin-1-yl)-2,4-dimethylnicotinic acid

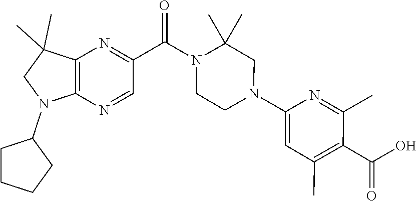 [SMILES: Cc1cc(N2CCN(C(=O)c3cnc4c(n3)C(C)(C)CN4C3CCCC3)C(C)(C)C2)nc(C)c1C(=O)O]

Example 183 was prepared according to general procedure (XI) starting from Example 182 (90 mg). The crude was purified by preparative HPLC (Column B, H₂O+0.1% HCOOH/MeCN+0.1% HCOOH 95:05 to 0:100) and freeze dried with HCl (0.1N in water) to obtain Example 183 (57 mg, 60%) as a yellow solid.

¹H-NMR (DMSO-d₆, 300 MHz) δ: 1.29 (s, 6H, 2CH₃); 1.51 (s, 6H, 2CH₃); 1.56-1.72 (m, 6H, 3CH₂); 1.81-1.88 (m, 2H, CH₂); 2.36 (s, 3H, CH₃); 2.55 (s, 3H, CH₃); 3.46 (s, 2H, N—CH₂); 3.69-3.72 (m, 2H, N—CH₂); 3.89 (t, J 5.2 Hz, 2H, N—CH₂); 3.95 (s, 2H, N—CH₂); 4.41 (m, J 7.3 Hz, 1H, N—CH); 6.90 (bs, 1H, Ar); 7.95 (s, 1H, Ar); COOH was not observed.

M/Z (M+H)⁺: 507.4.

### Compound 225: 3-chloro-N-(3-fluoro-4-methylphenyl)-N-(2-methylallyl)pyrazin-2-amine

Crude Compound 225 (1070 mg) was obtained as a brown oil according to general procedure (XVII) on a 500 mg scale, using 3-fluoro-4-methylaniline.

M/Z (M[³⁵Cl]+H)⁺: 292.2.

### Compound 226: 5-(3-fluoro-4-methylphenyl)-7,7-dimethyl-6,7-dihydro-5H-pyrrolo[2,3-b]pyrazine

Compound 226 was prepared according to general procedure (XVIII) starting from crude Compound 225 (970 mg). The crude was purified by flash chromatography (Interchim® 50 μm, CyHex 100% to CyHex/EtOAc 50:50) to obtain Compound 226 (385 mg, 45% over 2 steps) as an orange oil.

M/Z (M+H)⁺: 258.2

### Compound 227: 2-bromo-5-(3-fluoro-4-methylphenyl)-7,7-dimethyl-6,7-dihydro-5H-pyrrolo[2,3-b]pyrazine

Compound 227 (394 mg, 86%) was obtained as a yellow solid following general procedure (XVI) in MeCN, after purification by flash chromatography (Interchim® 50 μm, CyHex 100% to CyHex/EtOAc 70:30), starting from Compound 226 (350 mg).

M/Z (M[⁷⁹Br]+H)⁺: 336.2.

### Example 184: methyl 6-(4-(5-(3-fluoro-4-methylphenyl)-7,7-dimethyl-6,7-dihydro-5H-pyrrolo[2,3-b]pyrazine-2-carbonyl)-3,3-dimethylpiperazin-1-yl)-2,4-dimethylnicotinate

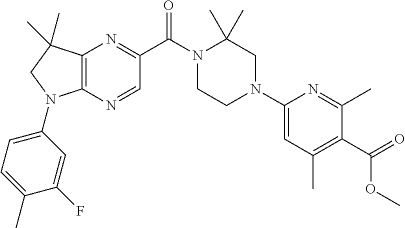 [SMILES: COC(=O)c1c(C)cc(N2CCN(C(=O)c3cnc4c(n3)C(C)(C)CN4c3ccc(C)c(F)c3)C(C)(C)C2)nc1C]

Example 184 was prepared according to general procedure (XIV) starting from Compound 227 (150 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 60:40) to obtain Example 184 (150 mg) as a yellow solid.

¹H-NMR (DMSO-d₆, 300 MHz) δ: 1.39 (s, 6H, 2CH₃); 1.50 (s, 6H, 2CH₃); 2.22 (s, 3H, CH₃); 2.24 (s, 3H, CH₃); 2.35 (s, 3H, CH₃); 3.53-3.56 (m, 2H, N—CH₂); 3.78 (s, 3H, O—CH₃); 3.78-3.83 (m, 2H, N—CH₂); 3.86-3.89 (m, 2H, N—CH₂); 3.96 (s, 2H, N—CH₂); 6.35 (s, 1H, Ar); 7.28-7.34 (m, 1H, Ar); 7.45-7.54 (m, 1H, Ar); 7.85-7.90 (m, 1H, Ar); 8.18 (s, 1H, Ar).

M/Z (M+H)⁺: 561.5

### Example 185: 6-(4-(5-(3-fluoro-4-methylphenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid

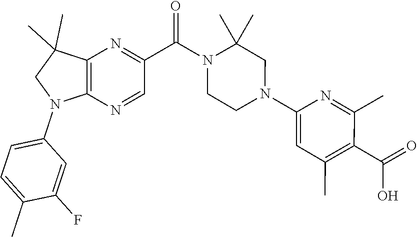 [SMILES: Cc1ccc(N2CC(C)(C)c3nc(C(=O)N4CCN(c5cc(C)c(C(=O)O)c(C)n5)CC4(C)C)cnc32)cc1F]

Example 185 was prepared according to general procedure (XI) starting from Example 184 (150 mg). The crude was purified by preparative HPLC (Column A, H₂O+0.1% HCOOH/MeCN+0.1% HCOOH 90:10 to 50:50) and freeze dried with HCl (0.1N in water) to obtain Example 185 (87 mg, 56% over 2 steps) as a yellow solid.

¹H-NMR (DMSO-d₆, 300 MHz) δ: 1.36 (s, 6H, (CH₃)₂); 1.52 (s, 6H, (CH₃)₂); 2.21 (s, 3H, CH₃); 2.33 (s, 3H, CH₃); 3.62-3.70 (m, 2H, N—CH₂); 3.83-3.90 (m, 2H, N—CH₂); 3.92-3.97 (m, 4H, N—CH₂); 6.75 (bs, 1H, Ar); 7.28-7.34 (m, 1H, Ar); 7.51-7.54 (m, 1H, Ar); 7.65-7.70 (m, 1H, Ar); 8.18 (s, 1H, Ar). One CH₃ and COOH were not observed.

M/Z (M+H)⁺: 547.4.

### Compound 228: 3-chloro-N-(4-chloro-3,5-difluorophenyl)-N-(2-methylallyl)pyrazin-2-amine

Crude Compound 228 (856 mg) was obtained as a brown oil according to general procedure (XVII) on a 683 mg scale, using 4-chloro-3,5-difluoroaniline.

M/Z (M[³⁵Cl]₂+H)⁺: 330.2

### Compound 229: 5-(4-chloro-3,5-difluorophenyl)-7,7-dimethyl-6,7-dihydro-5H-pyrrolo[2,3-b]pyrazine

Compound 229 was prepared according to general procedure (XVIII) starting from crude Compound 228 (856 mg). The crude was purified by flash chromatography (Interchim® 50 μm, CyHex 100% to CyHex/EtOAc 60:40) to obtain Compound 229 (586 mg, 65% over 2 steps) as a white solid.

M/Z (M[³⁵Cl]+H)⁺: 296.2

### Compound 230: 2-bromo-5-(4-chloro-3,5-difluorophenyl)-7,7-dimethyl-6,7-dihydro-5H-pyrrolo[2,3-b]pyrazine

Compound 230 (461 mg, 62%) was obtained as a white solid following general procedure (XVI) in MeCN, after purification by flash chromatography (Interchim® 50 μm, CyHex 100% to CyHex/EtOAc 70:30), starting from Compound 229 (586 mg).

M/Z (M[⁸¹Br][³⁵Cl]+H)⁺: 376.2.

### Example 186: methyl 6-(4-(5-(4-chloro-3,5-difluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinate

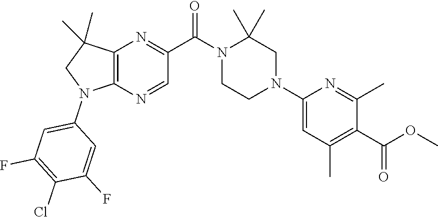 [SMILES: COC(=O)c1c(C)cc(N2CCN(C(=O)c3cnc4c(n3)C(C)(C)CN4c3cc(F)c(Cl)c(F)c3)C(C)(C)C2)nc1C]

Example 186 was prepared according to general procedure (XIV) starting from Compound 230 (150 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 186 (166 mg) as a yellow solid.

¹H-NMR (DMSO-d₆, 300 MHz) δ: 1.40 (s, 6H, 2CH₃); 1.50 (s, 6H, 2CH₃); 2.23 (s, 3H, CH₃); 2.35 (s, 3H, CH₃); 3.51- 3.58 (m, 2H, N—CH₂); 3.75-3.82 (m, 5H, O—CH₃N—CH₂); 3.87-3.92 (m, 2H, N—CH₂); 4.00 (s, 2H, N—CH₂); 6.37 (s, 1H, Ar); 7.88-7.98 (m, 2H, 2Ar); 8.26 (s, 1H, Ar).

M/Z (M[³⁵Cl]+H)⁺: 599.4

### Example 187: 6-(4-(5-(4-chloro-3,5-difluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid

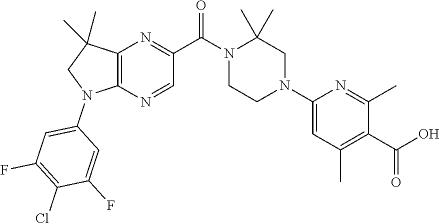 [SMILES: Cc1cc(N2CCN(C(=O)c3cnc4c(n3)C(C)(C)CN4c3cc(F)c(Cl)c(F)c3)C(C)(C)C2)nc(C)c1C(=O)O]

Example 187 was prepared according to general procedure (XI) starting from Example 186 (166 mg). The crude was purified by preparative HPLC (Column A, H₂O+0.1% HCOOH/MeCN+0.1% HCOOH 70:30 to 0:100) and freeze dried with HCl (0.1N in water) to obtain Example 187 (45 mg, 18% over 2 steps) as a white solid.

¹H-NMR (DMSO-d₆, 300 MHz) δ: 1.40 (s, 6H, 2CH₃); 1.53 (s, 6H, 2CH₃); 2.32 (s, 3H, CH₃); 2.47 (s, 3H, CH₃); 3.59- 3.69 (m, 2H, N—CH₂); 3.80-3.88 (m, 2H, N—CH₂); 3.87 (s, 2H, N—CH₂); 4.00 (s, 2H, N—CH₂); 6.50-6.82 (bs, 1H, Ar); 7.91-7.95 (m, 2H, 2Ar); 8.27 (s, 1H, Ar); COOH was not observed.

M/Z (M[³⁵Cl]+H)⁺: 585.4

### Compound 231: 3-chloro-N-(3-chloro-4-methylphenyl)-N-(2-methylallyl)pyrazin-2-amine

Crude Compound 231 (1010 mg) was obtained as a yellow oil according to general procedure (XVII) on a 500 mg scale, using 3-chloro-4-methylaniline.

M/Z (M[³⁵Cl]₂+H)⁺: 308.1

### Compound 232: 5-(3-chloro-4-methylphenyl)-7,7-dimethyl-6,7-dihydro-5H-pyrrolo[2,3-b]pyrazine

Compound 232 was prepared according to general procedure (XVIII) starting from crude Compound 231 (1010 mg). The crude was purified by flash chromatography (Interchim® 50 μm, CyHex 100% to CyHex/EtOAc 60:40) to obtain Compound 232 (419 mg, 46% over 2 steps) as an off-white solid.

M/Z (M[³⁵Cl]+H)⁺: 274.2

### Compound 233: 2-bromo-5-(3-chloro-4-methylphenyl)-7,7-dimethyl-6,7-dihydro-5H-pyrrolo[2,3-b]pyrazine

Compound 233 (354 mg, 73%) was obtained as a yellow solid following general procedure (XVI) in MeCN, after purification by flash chromatography (Interchim® 50 μm, CyHex 100% to CyHex/EtOAc 70:30), starting from Compound 232 (377 mg).

M/Z (M[⁸¹Br][³⁵Cl]+H)⁺: 354.2

### Example 188: methyl 6-(4-(5-(3-chloro-4-methylphenyl)-7,7-dimethyl-6,7-dihydro-5H-pyrrolo[2,3-b]pyrazine-2-carbonyl)-3,3-dimethylpiperazin-1-yl)-2,4-dimethylnicotinate

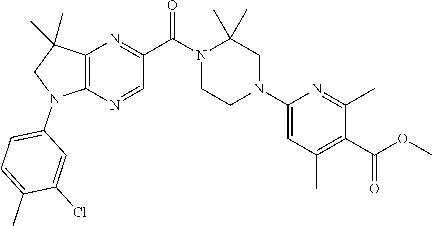 [SMILES: COC(=O)c1c(C)cc(N2CCN(C(=O)c3cnc4c(n3)C(C)(C)CN4c3ccc(C)c(Cl)c3)C(C)(C)C2)nc1C]

Example 188 was prepared according to general procedure (XIV) starting from Compound 233 (150 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 188 (189 mg) as a yellow solid.

¹H-NMR (DMSO-d₆, 300 MHz) δ: 1.39 (s, 6H, 2CH₃); 1.50 (s, 6H, 2CH₃); 2.23 (s, 3H, CH₃); 2.34 (s, 3H, CH₃); 2.35 (s, 3H, CH₃); 3.51-3.58 (m, 2H, N—CH₂); 3.78-3.85 (m, 5H, O—CH₃N—CH₂); 3.85-3.89 (m, 2H, N—CH₂); 3.99 (s, 2H, N—CH₂); 6.37 (s, 1H, Ar); 7.36-7.53 (m, 2H, 2Ar); 7.81-7.89 (m, 1H, Ar); 8.19-8.22 (m, 2H, 2Ar).

M/Z (M[³⁵Cl]+H)⁺: 577.5

### Example 189: 6-(4-(5-(3-chloro-4-methylphenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid

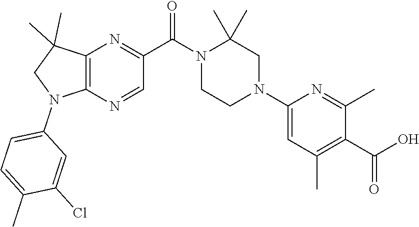 [SMILES: Cc1ccc(N2CC(C)(C)c3nc(C(=O)N4CCN(c5cc(C)c(C(=O)O)c(C)n5)CC4(C)C)cnc32)cc1Cl]

Example 189 was prepared according to general procedure (XI) starting from Example 188 (189 mg). The crude was purified by preparative HPLC (Column A, H₂O+0.1% HCOOH/MeCN+0.1% HCOOH 70:30 to 0:100) and freeze dried with HCl (0.1N in water) to obtain Example 189 (83 mg, 35% over 2 steps) as a yellow solid.

¹H-NMR (DMSO-d₆, 300 MHz) δ: 1.38 (s, 6H, 2×CH₃); 1.52 (s, 6H, 2×CH₃); 2.30 (s, 3H, CH₃); 2.33 (s, 3H, CH₃); 3.61-3.75 (m, 2H, N—CH₂); 3.82-3.88 (m, 2H, N—CH₂); 3.96 (s, 4H, 2N—CH₂); 6.62-6.95 (bs, 1H, Ar); 7.38 (d, J 8.6 Hz; 1H, Ar); 7.68 (d, J 8.6, 2.5 Hz; 1H, Ar); 8.09 (d, J 2.5 Hz; 1H, Ar); 8.20 (s, 1H, Ar); one CH₃ and COOH were not observed.

M/Z (M[³⁵Cl]+H)⁺: 563.4.

### Compound 234: 3-chloro-N-(3-chloro-4-fluorophenyl)-N-(2-methylallyl)pyrazin-2-amine

Crude Compound 234 (960 mg) was obtained as a yellow oil according to general procedure (XVII) on a 500 mg scale, using 3-chloro-4-fluoroaniline.

M/Z (M[³⁵Cl]₂+H)⁺: 312.2

### Compound 235: 5-(3-chloro-4-fluorophenyl)-7,7-dimethyl-6,7-dihydro-5H-pyrrolo[2,3-b]pyrazine

Compound 235 was prepared according to general procedure (XVIII) starting from crude Compound 234 (960 mg). The crude was purified by flash chromatography (Interchim® 50 μm, CyHex 100% to CyHex/EtOAc 60:40) to obtain Compound 235 (511 mg, 55% over 2 steps) as an off-white solid.

M/Z (M[³⁵Cl]+H)⁺: 278.1

### Compound 236: 2-bromo-5-(3-chloro-4-fluorophenyl)-7,7-dimethyl-6,7-dihydro-5H-pyrrolo[2,3-b]pyrazine

Compound 236 (504 mg, 77%) was obtained as a yellow solid following general procedure (XVI) in MeCN, after purification by flash chromatography (Interchim® 50 μm, CyHex 100% to CyHex/EtOAc 70:30), starting from Compound 235 (511 mg).

M/Z (M[⁸¹Br][³⁵Cl]+H)⁺: 358.1

### Example 190: methyl 6-(4-(5-(3-chloro-4-fluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinate

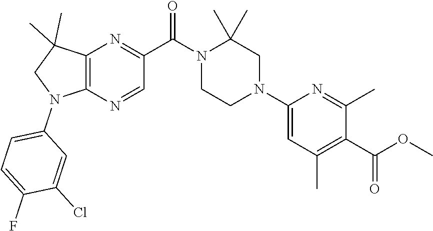 [SMILES: COC(=O)c1c(C)cc(N2CCN(C(=O)c3cnc4c(n3)C(C)(C)CN4c3ccc(F)c(Cl)c3)C(C)(C)C2)nc1C]

Example 190 was prepared according to general procedure (XIV) starting from Compound 236 (150 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 190 (122 mg) as a brown oil.

¹H-NMR (DMSO-d₆, 300 MHz) δ: 1.40 (s, 6H, 2CH₃); 1.50 (s, 6H, 2CH₃); 2.24 (s, 3H, CH₃); 2.35 (s, 3H, CH₃); 3.51- 3.58 (m, 2H, N—CH₂); 3.76-3.82 (m, 5H, O—CH₃N—CH₂); 3.85-3.89 (m, 2H, N—CH₂); 3.99 (s, 2H, N—CH₂); 6.34-6.40 (bs, 1H, Ar); 7.42 (d, J 8.6 Hz, 1H, Ar); 7.68 (d, J 8.6, 2.5 Hz, 1H, Ar); 8.10 (d, J 2.5 Hz, 1H, Ar); 8.19 (s, 1H, Ar).

M/Z (M[³⁵Cl]+H)⁺: 581.4

### Example 191: 6-(4-(5-(3-chloro-4-fluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid

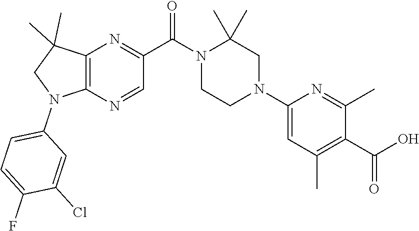 [SMILES: Cc1cc(N2CCN(C(=O)c3cnc4c(n3)C(C)(C)CN4c3ccc(F)c(Cl)c3)C(C)(C)C2)nc(C)c1C(=O)O]

Example 191 was prepared according to general procedure (XI) starting from Example 190 (122 mg). The crude was purified by preparative HPLC (Column A, H₂O+0.1% HCOOH/MeCN+0.1% HCOOH 70:30 to 0:100) and freeze dried with HCl (0.1N in water) to obtain Example 191 (43 mg, 18% over 2 steps) as a yellow solid.

¹H-NMR (DMSO-d₆, 300 MHz) δ: 1.40 (s, 6H, 2CH₃); 1.53 (s, 6H, 2CH₃); 2.34 (s, 3H, CH₃); 3.61-3.73 (m, 2H, N—CH₂); 3.82-3.91 (m, 2H, N—CH₂); 3.95 (s, 2H, N—CH₂); 4.00 (s, 2H, N—CH₂); 6.59-6.89 (bs, 1H, Ar); 7.44-7.56 (m, 1H, Ar); 7.80-7.88 (m; 1H, Ar); 8.18-8.24 (m; 1H, Ar); 8.22 (s, 1H, Ar); One CH₃ and COOH were not observed.

M/Z (M[³⁵Cl]+H)⁺: 567.4.

### Compound 237: 3-chloro-N-(3-chloro-4-(trifluoromethyl)phenyl)-N-(2-methylallyl)pyrazin-2-amine

Crude Compound 237 (780 mg) was obtained as a yellow oil according to general procedure (XVII) on a 500 mg scale, using 3-chloro-4-(trifluoromethyl)aniline.

M/Z (M[³⁵Cl]₂+H)⁺: 363.2

### Compound 238: 5-(3-chloro-4-(trifluoromethyl)phenyl)-7,7-dimethyl-6,7-dihydro-5H-pyrrolo[2,3-b]pyrazine

Compound 238 was prepared according to general procedure (XVIII) starting from crude Compound 237 (780 mg). The crude was purified by flash chromatography (Interchim® 50 μm, CyHex 100% to CyHex/EtOAc 50:50) to obtain Compound 238 (528 mg, 48% over 2 steps) as a white solid.

M/Z (M[³⁵Cl]+H)⁺: 328.3

### Compound 239: 2-bromo-5-(3-chloro-4-(trifluoromethyl)phenyl)-7,7-dimethyl-6,7-dihydro-5H-pyrrolo[2,3-b]pyrazine

Compound 239 (503 mg, 78%) was obtained as a white solid following general procedure (XVI) in MeCN without further purification starting from Compound 238 (520 mg).

M/Z (M[⁸¹Br][³⁵Cl]+H)⁺: 408.0

### Example 192: methyl 6-(4-(5-(3-chloro-4-(trifluoromethyl)phenyl)-7,7-dimethyl-6,7-dihydro-5H-pyrrolo[2,3-b]pyrazine-2-carbonyl)-3,3-dimethylpiperazin-1-yl)-2,4-dimethylnicotinate

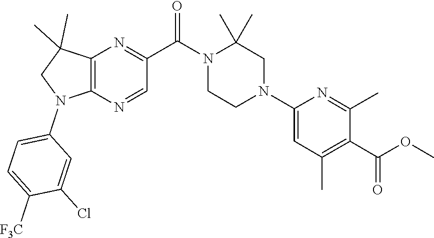 [SMILES: COC(=O)c1c(C)cc(N2CCN(C(=O)c3cnc4c(n3)C(C)(C)CN4c3ccc(C(F)(F)F)c(Cl)c3)C(C)(C)C2)nc1C]

Example 192 was prepared according to general procedure (XIV) starting from Compound 239 (150 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 20 μm, CyHex 100% to CyHex/EtOAc 30:70) to obtain Example 192 (112 mg, 48%) as a white solid.

¹H-NMR (DMSO-d₆, 300 MHz) δ: 1.40 (s, 6H, 2CH₃); 1.50 (s, 6H, 2CH₃); 2.24 (s, 3H, CH₃); 2.34 (s, 3H, CH₃); 3.52- 3.58 (m, 2H, N—CH₂); 3.75-3.82 (m, 5H, O—CH₃N—CH₂); 3.85-3.88 (m, 2H, N—CH₂); 3.99 (s, 2H, N—CH₂); 6.37 (s, 1H, Ar); 7.85-7.90 (m, 1H, Ar); 7.96-8.00 (m, 1H, Ar); 8.25 (s, 1H, Ar); 8.29-8.32 (m, 1H, Ar).

M/Z (M[35Cl]+H)⁺: 631.3

### Example 193: 6-(4-(5-(3-chloro-4-(trifluoromethyl)phenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid

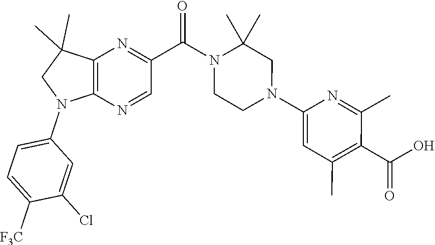 [SMILES: Cc1cc(N2CCN(C(=O)c3cnc4c(n3)C(C)(C)CN4c3ccc(C(F)(F)F)c(Cl)c3)C(C)(C)C2)nc(C)c1C(=O)O]

Example 193 was prepared according to general procedure (XI) starting from Example 192 (122 mg). The crude was purified by preparative HPLC (Column A, H₂O+0.1% HCOOH/MeCN+0.1% HCOOH 70:30 to 0:100) and freeze dried with HCl (0.1N in water) to obtain Example 193 (43 mg, 18% over 2 steps) as a yellow solid.

¹H-NMR (DMSO-d₆, 300 MHz) δ: 1.40 (s, 6H, (CH₃)₂); 1.53 (s, 6H, (CH₃)₂); 2.33 (s, 3H, CH₃); 3.60-3.71 (m, 2H, CH₂); 3.81-3.88 (m, 2H, CH₂); 3.92-3.97 (m, 2H, CH₂); 4.05 (s, 2H, CH₂); 6.77 (bs, 1H, Ar); 7.86-7.89 (m, 1H, Ar); 7.96-7.99 (m, 1H, Ar); 8.29 (s, 1H, Ar); 8.30-8.32 (m, 1H, Ar), One CH₃ and COOH were not observed

M/Z (M[³⁵Cl]+H)⁺: 617.3

### Compound 240: diethyl 1-(1-((tert-butoxycarbonyl)amino)-2-methylpropan-2-yl)-1H-pyrazole-3,5-dicarboxylrate

To a solution of diethyl 1H-pyrazole-3,5-dicarboxylate (3.0 g) in TH (30 mL) was added tert-butyl (2-hydroxy-2-methylpropyl) carbamate (5.35 a). Ph₃P (6.67 a) and DIAD (5.15 a). The reaction was heated at 50° C. for 1 hour under a nitrogen atmosphere. The mixture was quenched with H₂O (60 mL) and extracted with EtOAc (3*60 mL). The combined organic layer was washed with brine (50 mL), dried over Na₂SO₄ then concentrated. The crude was purified by flash chromatography (Petroleum ether 100% to Petroleum ether/EtOAc 80:20) to obtain Compound 240 (3.56 g, 65%) as a yellow oil.

M/Z (M+H)⁺: 284.2.

### Compound 241: ethyl 7,7-dimethyl-4-oxo-4,5,6,7-tetrahydropyrazolo[1,5-a]pyrazine-2-carboxylate

A solution of Compound 240 (3.56 g) and HCl (4M in dioxane, 46 mL) was stirred at 25° C. for 4 hours. The reaction mixture was treated with Na₂CO₃ (sat. aq.) to pH=10 and extracted with EtOAc (3*80 mL). The combined organic layers were washed with brine, dried over Na₂SO₄ and concentrated under reduced pressure to obtain Compound 241 (2.12 g, 96%) as a yellow solid.

¹H NMR (DMSO-d₆, 400 MHz) δ 1.30 (t, J 7.1 Hz, 3H, CH₂—CH₃); 1.50 (s, 6H, C(CH₃)₂); 3.49-3.53 (m, 2H, N—CH₂); 4.26-4.33 (m, 2H, N—CH₂), 7.09 (s, 1H, Ar), 8.44 (s, 1H, C(O)NH).

M/Z (M+H)⁺: 238.1.

### Compound 242: ethyl 7,7-dimethyl-4,5,6,7-tetrahydropyrazolo[1,5-a]pyrazine-2-carboxylate

A solution of Compound 241 (1.0 g) and BH₃·THF (1M in THF, 63 mL) was stirred at 25° C. for 16 hours, then quenched with 50 mL MeOH and refluxed for 1 hour. The mixture was then concentrated under reduced pressure, taken up in H₂O (50 mL), and extracted with EtOAc (3*50 mL). The combined organic layers were washed with brine, dried over Na₂SO₄ and concentrated under reduced pressure. The crude was purified by flash chromatography (Petroleum ether 100% to Petroleum ether/EtOAc 0:100) to obtain Compound 242 (0.45 g, 47%) as a white solid.

¹H NMR (DMSO-d₆, 400 MHz) δ 1.27 (t, J 7.1 Hz, 3H, CH₂—CH₃), 1.44 (s, 6H, C(CH₃)₂), 2.94 (s, 2H, N—CH₂); 3.88 (s, 2H, N—CH₂); 4.24 (q, J 7.1 Hz, 2H, CH₂—CH₃); 6.40 (s, 1H, Ar).

M/Z (M+H)⁺: 224.1

### Compound 243: ethyl 5-(4-chloro-3-fluorophenyl)-7,7-dimethyl-4,5,6,7-tetrahydropyrazolo[1,5-a]pyrazine-2-carboxylate

To a solution of Compound 242 (451 mg), in Dioxane (50 mL), was added 1-chloro-2-fluoro-4-iodobenzene (777 mg), RuPhos Pd G4 (172 mg) and cesium carbonate (1.32 g). The reaction was sparged with nitrogen then heated at 100° C. for 2 hours, filtered, and the filtrate was concentrated under reduced pressure. The residue was purified by flash chromatography (Petroleum ether 100% to Petroleum ether/EtOAc 60:40) to obtain Compound 243 (548 mg, 77%) as an off-white solid.

¹H NMR (DMSO-d₆, 400 MHz) δ 1.29 (t, J 7.1 Hz, 3H, CH₂—CH₃); 1.52 (s, 6H, C(CH₃)₂); 3.65-3.71 (m, 2H, N—CH₂); 4.27 (q, J 7.1 Hz, 2H, CH₂—CH₃); 4.45-4.51 (m, 2H, N—CH₂); 6.56 (s, 1H, Ar); 6.82-6.98 (m, 1H, Ar); 7.06-7.18 (m, 1H, Ar); 7.35-7.47 (m, 1H, Ar).

M/Z (M[³⁵Cl]+H)⁺: 352.1

### Compound 244: 5-(4-chloro-3-fluorophenyl)-7,7-dimethyl-4,5,6,7-tetrahydropyrazolo[1,5-a]pyrazine-2-carboxylic acid

To a solution of Compound 243 (528 mg) in THE (25 mL) was added LiOH (359 mg) and H₂O (5 mL). The reaction mixture was stirred at 25° C. for 48 hours, acidified with HCl (1M, aq.) to pH=5, then extracted with EtOAc (3*30 mL). The combined organic layers were washed with brine, dried over Na₂SO₄ and concentrated under reduced pressure to obtain Compound 244 (488 mg) as a white solid.

M/Z (M[³⁵Cl]+H)⁺: 324.1

### Example 194: methyl 2-(6-(4-(5-(4-chloro-3-fluorophenyl)-7,7-dimethyl-4,5,6,7-tetrahydropyrazolo[1,5-a]pyrazine-2-carbonyl)-3,3-dimethylpiperazin-1-yl)pyridin-3-yl)acetate

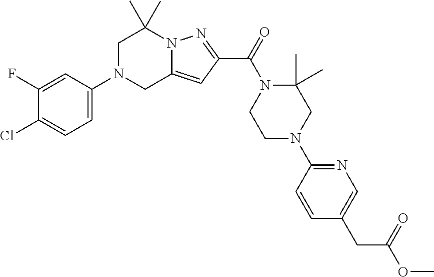 [SMILES: COC(=O)Cc1ccc(N2CCN(C(=O)c3cc4n(n3)C(C)(C)CN(c3ccc(Cl)c(F)c3)C4)C(C)(C)C2)nc1]

To a solution of Compound 244 (200 mg) in DMF (15 mL) was added HATU (352 mg) and DIPEA (240 mg) at 0° C.

The mixture was stirred for 20 minutes before adding Compound 111 (195 mg). The reaction mixture was stirred at 25° C. for 2 hours then diluted with H₂O (10 mL) and extracted with EtOAc (3*20 mL). The combined organic layers were washed with brine, dried over Na₂SO₄ and concentrated under reduced pressure. The residue was purified by flash chromatography (Petroleum ether 100% to Petroleum ether/EtOAc 30:70) to obtain Example 194 (381 mg) as a yellow solid.

¹H NMR (DMSO-d₆, 400 MHz) δ 1.47 (s, 6H, C(CH₃)₂); 1.52 (s, 6H), C(CH₃)₂); 3.48 (t, J 5.4 Hz, 2H, N—CH₂); 3.54 (s, 2H, CH₂C(O)); 3.61 (s, 3H, O—CH₃); 3.68 (s, 2H, N—CH₂); 3.84 (s, 2H, N—CH₂); 4.11 (t, J 5.4 Hz, 2H, N—CH₂); 4.50 (s, 2H, N—CH₂); 6.31 (s, 1H, Ar); 6.59 (d, J 8.8 Hz, 1H, Ar); 6.87-6.94 (m, 1H, Ar); 7.06-7.15 (m, 1H, Ar); 7.36-7.47 (m, 2H); 7.96 (d, J=2.1 Hz, 1H);

M/Z (M[³⁵Cl]+H)⁺: 569.2.

### Example 195: 2-(6-(4-(5-(4-chloro-3-fluorophenyl)-7,7-dimethyl-4,5,6,7-tetrahydropyrazolo[1,5-a]pyrazine-2-carbonyl)-3,3-dimethylpiperazin-1-yl)pyridin-3-yl)acetic acid

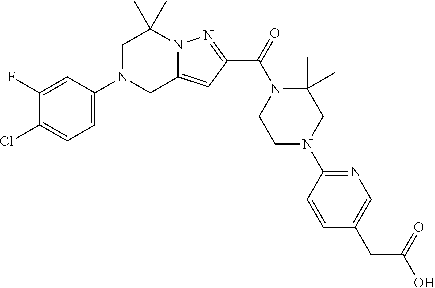 [SMILES: CC1(C)CN(c2ccc(CC(=O)O)cn2)CCN1C(=O)c1cc2n(n1)C(C)(C)CN(c1ccc(Cl)c(F)c1)C2]

To a solution of Example 194 (189 mg) in THE (30 mL) was added LiOH (152 mg) dissolved in water (6 mL) at 0° C. The reaction was stirred at 25° C. for 3 hours. The mixture was adjusted pH to 5 with HCl (aq. 1M) and extracted with EtOAc (3*30 mL). The combined organic layer was washed with brine (40 mL), dried over Na₂SO₄ and concentrated under reduced pressure. The crude was purified by Prep-HPLC (MeCN/H₂O with 0.05% HCOOH) to obtain Example 195 (163 mg, 46%) as a white solid.

¹H NMR (DMSO-d₆, 400 MHz) δ 1.48 (s, 6H, C(CH₃)₂); 1.53 (s, 6H), C(CH₃)₂); 3.43 (s, 2H, CH₂C(O)); 3.49 (t, J 5.4 Hz, 2H, N—CH₂); 3.69 (s, 2H, N—CH₂); 3.85 (s, 2H, N—CH₂); 4.11 (t, J 5.4 Hz, 2H, N—CH₂); 4.51 (s, 2H, N—CH₂); 6.32 (s, 1H, Ar); 6.59 (d, J 8.6 Hz, 1H, Ar); 6.89-6.96 (m, 1H, Ar); 7.08-7.16 (m, 1H, Ar); 7.36-7.50 (m, 2H, 2Ar); 7.95 (d, J=2.0 Hz, 1H, Ar); 12.26 (bs, 1H, COOH);

M/Z (M[³⁵Cl]+H)⁺: 555.2.

### Compound 245: methyl 5-bromo-6-((4-methylphenyl)sulfonamido)picolinate

To a solution of methyl 6-amino-5-bromopicolinate (5.00 g) in Pyridine (100 mL) was added 4-methylbenzenesulfonyl chloride (4.95 g), and the reaction was stirred at 120° C. After 6 hours, H₂O (300 mL) was added. The mixture was extracted with EtOAc (2*300 mL), the combined organic layers were washed with brine (300 mL), dried over Na₂SO₄ and concentrated under reduced pressure. The residue was purified by flash chromatography (Petroleum ether/EtOAc 50:50) to obtain Compound 245 (3.00 g, 36%) as a yellow solid.

M/Z (M[⁷⁹Br]+H)⁺: 385.0.

### Compound 246: methyl 5-bromo-6-((4-methyl-N-(2-methylallyl)phenyl)sulfonamido)picolinate

To a solution of Compound 245 (3.40 g) in THE (80 mL) was added triphenylphosphine (3.47 g) and 2-methylprop-2-en-1-ol (764 mg), followed by Diisopropyl azodicarboxylate (3.57 g), slowly mixture at 0° C. The reaction was stirred at 25° C. for 3 hours, then, H₂O (200 mL) was added. The mixture was extracted with EtOAc (2*200 mL), the combined organic layers were washed with brine (200 mL), dried over anhydrous Na₂SO₄ and concentrated under reduced pressure The residue was purified by flash chromatography (Petroleum ether/EtOAc 75:25) to obtain Compound 246 (2.20 g, 56%) as a yellow solid.

M/Z (M[⁷⁹Br]+H)⁺: 439.0.

### Compound 247: methyl 3-(4-chloro-3-fluorobenzyl)-3-methyl-1-tosyl-2,3-dihydro-1H-pyrrolo[2,3-b]pyridine-6-carboxylate

To a solution of Compound 246 (2.00 g) in DMF (30 mL) was added K₂CO₃ (2.52 g) and (4-chloro-3-fluorophenyl)boronic acid (1.59 g) and Pd₂(dba)₃ (2.08 g). The reaction was stirred at 100° C. for 3 hours, then H₂O (100 mL) was added, and the mixture was extracted with EtOAc (2*100 mL). The combined organic layers were washed with brine (100 mL), dried over Na₂SO₄ and concentrated under reduced pressure. The residue was purified by flash chromatography (Petroleum ether/EtOAc 75:25) to obtain Compound 247 (2.00 g, 89%) as a white solid.

¹H NMR (DMSO-d₆, 400 MHz,) δ 1.33 (s, 3H, C(CH₃)); 2.33 (s, 3H, Ar—CH₃); 2.90-3.01 (m, 2H, Ar—CH₂); 3.67 (d, J=10.1 Hz, 1H, N—CHₐH_{b}); 3.91 (s, 3H, O—CH₃); 4.09 (d, J=10.1 Hz, 1H, N—CHₐH_{b}); 6.73 (dd, J=8.2, 1.8 Hz, 1H, Ar); 6.98 (dd, J=10.6, 1.8 Hz, 1H, Ar); 7.26-7.38 (m, 2H); 7.63-7.75 (m, 3H); 7.90 (d, J=8.4 Hz, 2H);

M/Z (M[³⁵Cl]+H)⁺: 489.1.

### Compound 248: methyl 3-(4-chloro-3-fluorobenzyl)-3-methyl-2,3-dihydro-1H-pyrrolo[2,3-b]pyridine-6-carboxylate

To a solution of Compound 247 (1.60 g) in H₂SO₄ (20 mL) was heated at 100° C. for 3 hours, then the mixture was added slowly in ice water (200 mL) and extracted with EtOAc (2*200 mL), the combined organic layers were washed with brine (200 mL), dried over Na₂SO₄ and concentrated under reduced pressure. The residue was purified by flash chromatography (Petroleum ether/EtOAc 50:50) to obtain Compound 248 (650 mg, 59%) as a white solid.

¹H NMR (DMSO-d₆, 400 MHz,) δ 1.27 (s, 3H, C(CH₃)); 2.83-2.95 (m, 2H, Ar—CH₂); 3.14 (d, J=9.1 Hz, 1H, N—CHₐH_{b}); 3.51 (d, J=9.5 Hz, 1H, N—CHₐH_{b}); 3.78 (s, 3H, O—CH₃); 6.74 (s, 1H, Ar); 6.84-6.88 (m, 1H, Ar); 7.05-7.11 (m, 1H, Ar); 7.19-7.30 (m, 2H, 2Ar); 7.40-7.46 (m, 1H, N—H);

M/Z (M[³⁵Cl]+H)⁺: 335.0.

### Compound 249: 3-(4-chloro-3-fluorobenzyl)-1-isobutyl-3-methyl-2,3-dihydro-1H-pyrrolo[2,3-b]pyridine-6-carboxylic acid

To a solution of Compound 248 (500 mg) in Toluene (10 mL) was added Potassium 2-methylpropan-2-olate (1.68 g) and 1-bromo-2-methylpropane (1.02 g). The mixture was purged with argon and heated at 60° C. for 3 hours then treated with H₂O (100 mL) and the mixture was extracted with EtOAc (2*100 mL), the combined organic layers were washed with brine (100 mL), dried over Na₂SO₄ and concentrated under reduced pressure. The residue was purified by flash chromatography (Petroleum ether/EtOAc 33:67) to obtain Compound 249 (300 mg, 54%) as a white solid.

M/Z (M[³⁵Cl]+H)⁺: 377.1.

### Example 196: methyl 2-(6-(4-(3-(4-chloro-3-fluorobenzyl)-1-isobutyl-3-methyl-2,3-dihydro-1H-pyrrolo[2,3-b]pyridine-6-carbonyl)-3,3-dimethylpiperazin-1-yl)pyridin-3-yl)acetate

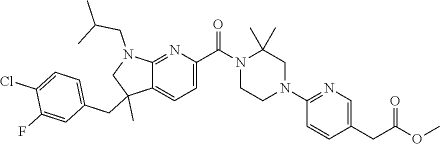 [SMILES: COC(=O)Cc1ccc(N2CCN(C(=O)c3ccc4c(n3)N(CC(C)C)CC4(C)Cc3ccc(Cl)c(F)c3)C(C)(C)C2)nc1]

To a solution of Compound 249 (300 mg) in DMF (10 mL) was added HATU (454 mg) and DIPEA (309 mg) at 0° C.

The mixture was stirred for 20 minutes before adding Compound 111 (252 mg). The reaction mixture was stirred at 25° C. for 2 hours then diluted with H₂O (50 mL) and extracted with EtOAc (2*50 mL). The combined organic layers were washed with brine, dried over Na₂SO₄ and concentrated under reduced pressure. The residue was purified by flash chromatography (Petroleum ether/EtOAc 75:25) to obtain Example 196 (300 mg, 60%) as a yellow solid.

¹H NMR (CDCl₃, 400 MHz) δ 0.79-0.90 (m, 6H, CH—(CH₃)₂); 1.32-1.36 (m, 3H, C—CH₃); 1.59-1.62 (m, 6H, C—(CH₃)₂); 1.81-1.89 (m, 1H, CH—(CH₃)₂); 2.74-2.91 (m, 3H, Ar—CH₂N—CHₐH_{b}); 3.08-3.14 (m, 1H, N—CHₐH_{b}); 3.19-3.27 (m, 1H, N—CHₐH_{b}); 3.42-3.48 (m, 1H, N—CHₐH_{b}); 3.49 (s, 2H, CH₂C(O)); 3.52-3.56 (m, 2H, N—CH₂); 3.69 (s, 3H, O—CH₃); 3.80-3.87 (m, 4H, 2N—CH₂); 6.42 (d, J=8.6 Hz, 1H, Ar); 6.70-6.79 (m, 3H, 3Ar); 6.99 (d, J=7.2 Hz, 1H, Ar); 7.21-7.25 (m, 1H, Ar); 7.42-7.49 (m, 1H, Ar); 8.04 (d, J=2.1 Hz, 1H, Ar);

M/Z (M[³⁵Cl]+H)⁺: 622.3.

### Example 197: 2-(6-(4-(3-(4-chloro-3-fluorobenzyl)-1-isobutyl-3-methyl-2,3-dihydro-1H-pyrrolo[2,3-b]pyridine-6-carbonyl)-3,3-dimethylpiperazin-1-yl)pyridin-3-yl)acetic acid

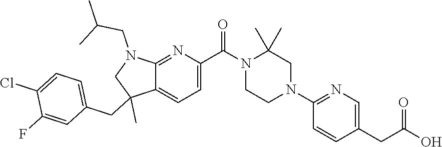 [SMILES: CC(C)CN1CC(C)(Cc2ccc(Cl)c(F)c2)c2ccc(C(=O)N3CCN(c4ccc(CC(=O)O)cn4)CC3(C)C)nc21]

To a solution of Example 196 (300 mg) in THE (10 mL) was added LiOH (35 mg) dissolved in water (2 mL) at 0° C. The reaction was stirred at 25° C. for 3 hours. The mixture was adjusted pH to 6 with HCl (aq. 1M) and extracted with EtOAc (2*10 mL). The combined organic layer were dried over Na₂SO₄ and concentrated under reduced pressure. The crude was purified by Prep-HPLC (MeCN/H₂O with 0.05% HCOOH) to obtain Example 197 (200 mg, 68%) as a white solid.

¹H NMR (CDCl₃, 400 MHz) δ 0.65-0.80 (m, 6H, CH—(CH₃)₂); 1.34 (s, 3H, C—CH₃); 1.42-1.50 (m, 6H, C—(CH₃)₂); 1.74-1.79 (m, 1H, CH—(CH₃)₂); 2.76-2.90 (m, 3H, Ar—CH₂N—CHₐH_{b}); 3.03-3.14 (m, 2H, N—CHₐH_{b}); 3.40-3.44 (m, 4H, CH₂C(O)N—CH₂); 3.54-3.67 (m, 3H, N—CHₐH_{b}, N—CH₂); 3.79-3.85 (m, 2H, N—CH₂); 6.51 (d, J 8.7 Hz, 1H, Ar); 6.60 (d, J 7.1 Hz, 1H, Ar); 6.82-6.88 (m, 1H, Ar); 6.99-7.04 (m, 1H, Ar); 7.32 (d, J 7.2 Hz, 1H, Ar); 7.37-7.47 (m, 2H, 2Ar); 7.94 (d, J 2.2 Hz, 1H, Ar); 12.26 (bs, 1H, COOH);

M/Z (M[³⁵Cl]+H)⁺: 608.3

### Compound 250: 1-(3-chloropyridin-2-yl)-3-oxocyclobutane-1-carbonitrile

To a solution of Compound 181 (800 mg) and RuCl₃—H₂O (194 mg) in a MeCN/DCM/H₂O mixture (2:2:3, 14 mL) was added NalO₄ at 0° C. portionwise. The mixture was stirred at 25° C. for 15 hours then diluted in H₂O (50 mL) and extracted with DCM (3*50 mL). The combined organic layers were dried over MgSO₄ and concentrated under reduced pressure to obtain Compound 250 (786 mg, 97%) as a black solid.

M/Z (M[³⁵Cl]+H)⁺: 207.1

### Compound 251: 1-(3-chloropyridin-2-yl)-3-hydroxycyclobutane-1-carbonitrile

To a solution of Compound 250 (786 mg) in MeOH (10 mL) at 0° C. was added NaBH₄ (216 mg) portionwise. The reaction mixture was stirred at 0° C. for 30 minutes then quenched in NH₄Cl (sat. aq, 50 mL) and extracted with DCM (3*50 mL). The combined organic layers were dried over MgSO₄ and concentrated under reduced pressure to obtain Compound 251 (719 mg, 91%, 6:4 diastereoisomeric mixture) as a brown oil.

M/Z (M[³⁵Cl]+H)⁺: 209.1

### Compound 252: 1-(3-chloropyridin-2-yl)-3-methoxycyclobutane-1-carbonitrile

Compound 252 was prepared according to general procedure (XIII) starting from Compound 251 (719 mg) and Iodomethane (2.0 eq), and using NaH (60% in mineral oil, 2.0 eq) in THF. The crude was purified by flash chromatography (Interchim® 50 μm, CyHex 100% to CyHex/EtOAc 20:80) to obtain Compound 252 (654 mg, 85%, 6:4 diastereoisomeric mixture) as a clear oil.

M/Z (M[³⁵Cl]+H)⁺: 223.1

### Compound 253: (1-(3-chloropyridin-2-yl)-3-methoxycyclobutyl)methanamine

Compound 253 (369 mg) was obtained as a clear oil following general procedure (XVa), starting from Compound 252 (719 mg).

M/Z (M[³⁵Cl]+H⁺): 227.1

### Compound 254: (minor diastereoisomere) 1′-(4-chloro-3-fluorophenyl)-3-methoxy-1′,2′-dihydrospiro[cyclobutane-1,3′-pyrrolo[3,2-b]pyridine]

### And Compound 255: (major diastereoisomere) 1′-(4-chloro-3-fluorophenyl)-3-methoxy-1′,2′-dihydrospiro[cyclobutane-1,3′-pyrrolo[3,2-b]pyridine]

Compound 254 and Compound 255 were prepared according to general procedure (Xc) from Compound 253 (361 mg) and 4-bromo-1-chloro-2-fluorobenzene. The crude was purified by flash chromatography (Interchim® 50 μm, CyHex 100% to CyHex/EtOAc 70:30) to obtain Compound 254 (88 mg, 8% over 2 steps) as a white solid and Compound 255 (125 mg, 12% over 2 steps) as a white solid.

Compound 254: M/Z (M[³⁵Cl]+H⁺): 319.3.

Compound 255: M/Z (M[³⁵Cl]+H⁺): 319.3.

### Compound 256: (major diastereoisomere) 5′-bromo-1′-(4-chloro-3-fluorophenyl)-3-methoxy-1′,2′-dihydrospiro[cyclobutane-1,3′-pyrrolo[3,2-b]pyridine]

Compound 256 was prepared according to general procedure (XVI) in MeCN from Compound 255 (125 mg). The crude was purified by flash chromatography (Interchim® 50 μm, CyHex 100% to CyHex/EtOAc 70:30) to obtain Compound 256 (123 mg, 79%) as a clear oil.

M/Z (M[³⁵Cl][⁸¹Br]+H)⁺: 399.1.

### Example 198: (major diastereoisomere) methyl 6-(4-(1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinate

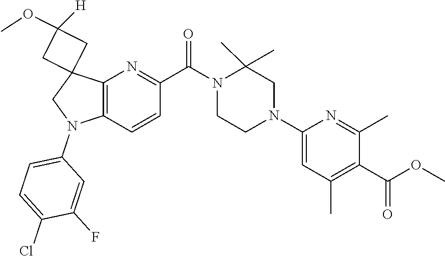

Example 198 was prepared according to general procedure (XIV) starting from Compound 256 (123 mg) and Compound 10 (1.5 eq), and using triethylamine (4.0 eq), Mo(CO)₆ (2.0 eq), XantPhos Pd G4 (0.13 eq), and DBU (5.8 eq). The crude was purified by flash chromatography (Interchim® 50 μm, CyHex 100% to CyHex/EtOAc 40:60) to obtain Example 198 (93 mg) as a brown solid.

¹H-NMR (DMSO-d₆, 300 MHz) δ: 1.51 (s, 6H, C(CH₃)₂); 2.25 (s, 3H, ArCH₃); 2.36 (s, 3H, ArCH₃); 2.37-2.45 (m, 2H, 2CHₐH_{b}); 3.23 (s, 3H, O—CH₃); 3.59-3.65 (m, 2H, N—CH₂—CH₂); 3.79 (s, 3H, C(O)OCH₃); 3.85-3.89 (m, 2H, N—CH₂—CH₂); 3.93 (s, 2H, N—CH₂); 3.98-4.13 (m, 1H, OCH); 4.21 (s, 2H, N—CH₂); 6.35 (s, 1H, Ar); 7.19 (dd, J 8.9, 2.0 Hz, 1H, Ar); 7.32 (dd, J 11.8, 2.5 Hz, 1H, Ar); 7.38 (d, J 8.5 Hz, 1H, Ar); 7.53-7.60 (m, 2H, 2Ar); 2CHₐH_{b} were not observed.

M/Z (M[³⁵Cl]+H⁺): 622.4.

### Example 199: (major diastereoisomere) 6-(4-(1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid

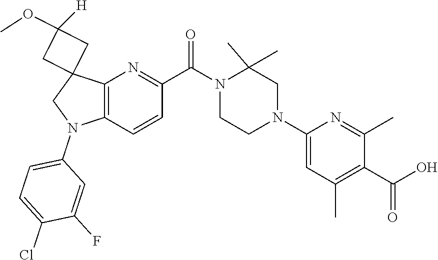 [SMILES: COC1CC2(C1)CN(c1ccc(Cl)c(F)c1)c1ccc(C(=O)N3CCN(c4cc(C)c(C(=O)O)c(C)n4)CC3(C)C)nc12]

Example 199 was prepared according to general procedure (XI) starting from Example 198 (93 mg). The crude was purified by preparative HPLC (Column A, H₂O+0.1% HCOOH/MeCN+0.1% HCOOH 65:35 to 0:100) and freeze dried with HCl (0.1N in water) to obtain Example 199 (44 mg, 22% over 2 steps) as a yellow solid.

¹H-NMR (DMSO-d₆, 300 MHz) δ: 1.54 (s, 6H, C(CH₃)₂); 2.33 (s, 3H, ArCH₃); 2.36-2.45 (m, 2H, 2CHₐH_{b}); 2.52-2.58 (m, 2H, 2CHₐH_{b}); 3.22 (s, 3H, O—CH₃); 3.66-3.75 (m, 2H, N—CH₂—CH₂); 3.85-3.94 (m, 2H, N—CH₂—CH₂); 3.98 (s, 2H, N—CH₂); 3.95-4.11 (m, 1H, OCH); 4.20 (s, 2H, N—CH₂); 6.66 (s, 1H, Ar); 7.19 (dd, J 8.9, 2.0 Hz, 1H, Ar); 7.33 (dd, J 11.8, 2.5 Hz, 1H, Ar); 7.38 (d, J 8.5 Hz, 1H, Ar); 7.53-7.60 (m, 2H, 2Ar); One ArCH₃ and COOH were not observed.

M/Z (M[³⁵Cl]+H⁺): 608.4.

### Compound 257: (minor diastereoisomere) 5′-bromo-1′-(4-chloro-3-fluorophenyl)-3-methoxy-1′,2′-dihydrospiro[cyclobutane-1,3′-pyrrolo[3,2-b]pyridine]

Compound 257 was prepared according to general procedure (XVI) in MeCN from Compound 254 (88 mg). The crude was purified by flash chromatography (Interchim® 50 μm, CyHex 100% to CyHex/EtOAc 70:30) to obtain Compound 257 (64 mg, 58%) as a clear oil.

M/Z (M[³⁵Cl][⁸¹Br]+H)⁺: 399.1.

### Example 200: (minor diastereoisomere) methyl 6-(4-(1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinate

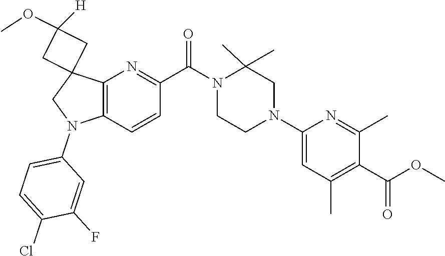

Example 200 was prepared according to general procedure (XIV) starting from Compound 257 (123 mg) and Compound 10 (1.5 eq), and using triethylamine (4.0 eq), Mo(CO)₆ (2.0 eq), XantPhos Pd G4 (0.13 eq), and DBU (5.8 eq). The crude was purified by flash chromatography (Interchim® 50 μm, CyHex 100% to CyHex/EtOAc 40:60) to obtain Example 200 (46 mg) as a brown solid.

¹H-NMR (DMSO-d₆, 300 MHz) δ: 1.51 (s, 6H, C(CH₃)₂); 2.25 (s, 3H, ArCH₃); 2.26-2.29 (m, 2H, 2CHₐH_{b}); 2.34 (s, 3H, ArCH₃); 2.61-2.70 (m, 2H, 2CHₐH_{b}); 3.24 (s, 3H, O—CH₃); 3.59-3.65 (m, 2H, N—CH₂—CH₂); 3.79 (s, 3H, C(O)OCH₃); 3.83-3.96 (m, 4H, N—CH₂—CH₂, N—CH₂); 4.17 (s, 2H, N—CH₂); 4.22-4.38 (m, 1H, OCH); 6.36 (s, 1H, Ar); 7.17 (dd, J 8.6, 2.0 Hz, 1H, Ar); 7.32 (dd, J 11.8, 2.6 Hz, 1H, Ar); 7.39 (d, J 8.5 Hz, 1H, Ar); 7.54-7.61 (m, 2H, 2Ar);

M/Z (M[³⁵Cl]+H⁺): 622.4.

### Example 201: (minor diastereoisomere) 6-(4-(1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid

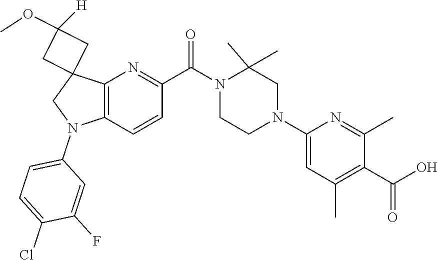 [SMILES: COC1CC2(C1)CN(c1ccc(Cl)c(F)c1)c1ccc(C(=O)N3CCN(c4cc(C)c(C(=O)O)c(C)n4)CC3(C)C)nc12]

Example 201 was prepared according to general procedure (XI) starting from Example 200 (46 mg). The crude was purified by preparative HPLC (Column A, H₂O+0.1% HCOOH/MeCN+0.1% HCOOH 70:30 to 0:100) and freeze dried with HCl (0.1N in water) to obtain Example 200 (20 mg, 19% over 2 steps) as a yellow solid.

¹H-NMR (DMSO-d₆, 300 MHz) δ: 1.55 (s, 6H, C(CH₃)₂); 2.20-2.30 (m, 2H, 2CHₐH_{b}); 2.33 (s, 3H, ArCH₃); 2.61-2.71 (m, 2H, 2CHₐH_{b}); 3.23 (s, 3H, O—CH₃); 3.68-3.78 (m, 2H, N—CH₂—CH₂); 3.91-3.97 (m, 4H, N—CH₂N—CH₂—CH₂); 4.15 (s, 2H, N—CH₂); 4.23-4.36 (m, 1H, OCH); 6.71 (s, 1H, Ar); 7.23-7.33 (m, 1H, Ar); 7.36-7.43 (m, 1H, Ar); 7.48-7.58 (m, 2H, 2Ar); One ArCH₃ and COOH were not observed.

M/Z (M[³⁵Cl]+H⁺): 608.3.

### Example 202: methyl 2-(6-(4-(5-(3-fluoro-4-methylphenyl)-7,7-dimethyl-6,7-dihydro-5H-pyrrolo[2,3-b]pyrazine-2-carbonyl)-3,3-dimethylpiperazin-1-yl)pyridin-3-yl)acetate

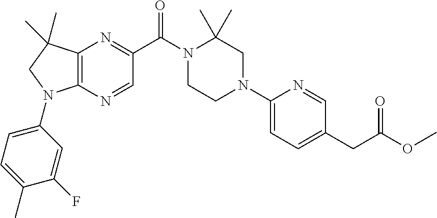 [SMILES: COC(=O)Cc1ccc(N2CCN(C(=O)c3cnc4c(n3)C(C)(C)CN4c3ccc(C)c(F)c3)C(C)(C)C2)nc1]

Example 202 was prepared according to general procedure (XIV) starting from Compound 227 (150 mg) and Compound 111 (1.5 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 60:40) to obtain Example 202 (164 mg) as a yellow oil.

¹H-NMR (DMSO-d₆, 300 MHz) δ: 1.38 (s, 6H, (CH₃)₂); 1.50 (s, 6H, (CH₃)₂); 2.21 (s, 3H, ArCH₃); 3.48-3.53 (m, 4H, CH₂C(O)N—CH₂); 3.59-3.66 (m, 4H, 2N—CH₂); 3.78-3.88 (m, 3H, O—CH₃); 3.96 (s, 2H, N—CH₂); 7.29-7.32 (m, 1H, Ar); 7.35-7.43 (m, 1H, Ar); 7.49-7.53 (m, 1H, Ar); 7.52-7.55 (m, 1H, Ar); 7.96-8.01 (m, 2H, 2Ar); 8.18 (s, 1H, Ar);

M/Z (M+H)⁺: 547.4

### Example 203: 2-(6-(4-(5-(3-fluoro-4-methylphenyl)-7,7-dimethyl-6,7-dihydro-5H-pyrrolo[2,3-b]pyrazine-2-carbonyl)-3,3-dimethylpiperazin-1-yl)pyridin-3-yl)acetic acid

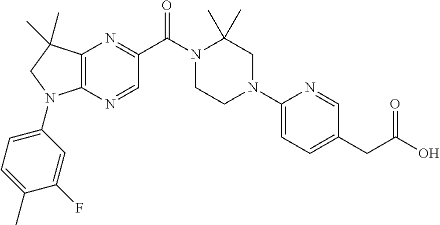 [SMILES: Cc1ccc(N2CC(C)(C)c3nc(C(=O)N4CCN(c5ccc(CC(=O)O)cn5)CC4(C)C)cnc32)cc1F]

Example 203 was prepared according to general procedure (VII) starting from Example 202 (142 mg). The crude was purified by preparative HPLC (Column B, H₂O+0.1% HCOOH/MeCN+0.1% HCOOH 65:35 to 0:100) and freeze dried with HCl (0.1N in water) to obtain Example 203 (90 mg, 35% over 2 steps) as a yellow solid.

¹H-NMR (DMSO-d₆, 300 MHz) δ: 1.36 (s, 6H, (CH₃)₂); 1.55 (s, 6H, (CH₃)₂); 2.20 (s, 3H, ArCH₃); 3.66 (s, 2H, C(O)CH₂); 3.71-3.77 (m, 2H, N—CH₂); 3.88-3.91 (m, 2H, N—CH₂); 3.92 (s, 2H, N—CH₂); 4.04 (s, 2H, N—CH₂); 7.27-7.33 (m, 1H, Ar); 7.38-7.42 (m, 1H, Ar); 7.49-7.53 (m, 1H, Ar); 7.64-7.69 (m, 1H, Ar); 7.96-8.01 (m, 2H, 2Ar); 8.20 (s, 1H, Ar). One COOH was not observed.

M/Z (M+H)⁺: 533.4

### Example 204: methyl 2-(6-(4-(5-(3-chloro-4-fluorophenyl)-7,7-dimethyl-6,7-dihydro-5H-pyrrolo[2,3-b]pyrazine-2-carbonyl)-3,3-dimethylpiperazin-1-yl)pyridin-3-yl)acetate

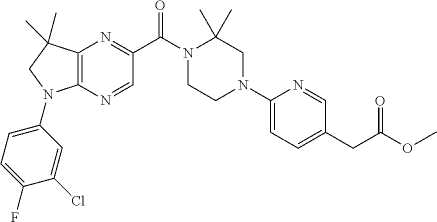 [SMILES: COC(=O)Cc1ccc(N2CCN(C(=O)c3cnc4c(n3)C(C)(C)CN4c3ccc(F)c(Cl)c3)C(C)(C)C2)nc1]

Example 204 was prepared according to general procedure (XIV) starting from Compound 236 (150 mg) and Compound 111 (1.5 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 204 (144 mg) as a yellow oil.

¹H-NMR (DMSO-d₆, 300 MHz) δ: 1.39 (s, 6H, 2CH₃); 1.50 (s, 6H, 2CH₃); 3.50-3.60 (m, 4H, N—CH₂CH₂C(O)); 3.64 (s, 3H, O—CH₃); 3.77-3.85 (m, 4H, 2N—CH₂); 3.98 (s, 2H, N—CH₂); 7.41-7.51 (m, 3H, 3Ar); 7.82-7.85 (m, 1H, Ar); 7.94-7.96 (m, 1H, Ar); 8.17-8.20 (m, 2H, 2Ar).

M/Z (M[³⁵Cl]+H)⁺: 567.3

### Example 205: 2-(6-(4-(5-(3-chloro-4-fluorophenyl)-7,7-dimethyl-6,7-dihydro-5H-pyrrolo[2,3-b]pyrazine-2-carbonyl)-3,3-dimethylpiperazin-1-yl)pyridin-3-yl)acetic acid

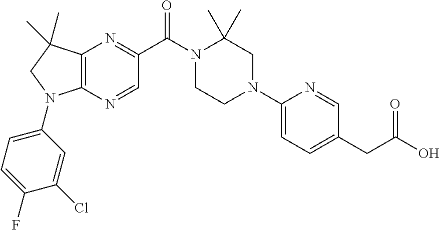 [SMILES: CC1(C)CN(c2ccc(F)c(Cl)c2)c2ncc(C(=O)N3CCN(c4ccc(CC(=O)O)cn4)CC3(C)C)nc21]

Example 205 was prepared according to general procedure (VII) starting from Example 204 (148 mg). The crude was purified by preparative HPLC (Column B, H₂O+0.1% HCOOH/MeCN+0.1% HCOOH 65:35 to 0:100) and freeze dried with HCl (0.1N in water) to obtain Example 205 (96 mg, 39% over 2 steps) as a yellow solid.

¹H-NMR (DMSO-d₆, 300 MHz) δ: 1.40 (s, 6H, 2CH₃); 1.56 (s, 6H, 2CH₃); 3.67 (s, 2H, CH₂C(O)); 3.70-3.79 (m, 2H, N—CH₂); 3.87-3.96 (m, 2H, N—CH₂); 3.99 (s, 2H, N—CH₂); 4.01 (s, 2H, N—CH₂); 7.34-7.45 (m, 1H, Ar); 7.47-7.53 (m, 1H, Ar); 7.82-7.87 (m, 1H, Ar); 7.95-8.02 (m, 1H, Ar); 8.17-8.26 (m, 2H, 2Ar); One COOH was not observed.

M/Z (M[³⁵Cl]+H)⁺: 553.3

### Compound 258: methyl 1-(3-chloropyrazin-2-yl)cyclopentane-1-carboxylate

Compound 258 was prepared according to general procedure (Ib) from dichloropyrazine (1.85 g) and methyl cyclopentanecarboxylate. The crude Compound 258 (3.40 g) obtained as an orange oil was directly engaged in the following step.

M/Z (M[³⁵Cl]+H)⁺: 241.0.

### Compound 259: (1-(3-chloropyrazin-2-yl)cyclopentyl)methanol

Compound 259 was prepared according to general procedure (XX) from Compound 258 (1.00 g). The crude was purified by flash chromatography (Interchim® 50 μm, CyHex 100% to CyHex/EtOAc 60:40) to obtain Compound 259 (0.24 g, 32% over 2 steps) as a clear oil.

M/Z (M[³⁵Cl]+H)⁺: 213.1.

### Compound 260: 1-(3-chloropyrazin-2-yl)cyclopentane-1-carbaldehyde

Compound 260 was prepared according to general procedure (XXI) from Compound 259 (260 mg). The crude was purified by flash chromatography (Interchim® 20 μm, CyHex 100% to CyHex/EtOAc 60:40) to obtain Compound 260 (169 mg, 66%) as a clear oil.

M/Z (M[³⁵Cl]+H)⁺: 211.1.

### Compound 261: 4-chloro-N-((1-(3-chloropyrazin-2-yl)cyclopentyl)methyl)-3-fluoroaniline

Compound 261 was prepared according to general procedure (XXII) from Compound 260 (165 mg) and 4-chloro-3-fluoroaniline. The crude was purified by flash chromatography (Interchim® 20 μm, CyHex 100% to CyHex/EtOAc 60:40) to obtain Compound 261 (147 mg, 55%) as a clear oil.

M/Z (M[³⁵Cl]₂+H)⁺: 340.2.

### Compound 262: 5′-(4-chloro-3-fluorophenyl)-5′,6′-dihydrospiro[cyclopentane-1,7′-pyrrolo[2,3-b]pyrazine]

Compound 262 was prepared according to general procedure (XXIII) from Compound 261 (145 mg). The crude was purified by flash chromatography (Interchim® 20 μm, CyHex 100% to CyHex/EtOAc 70:30) to obtain Compound 262 (100 mg, 77%) as a white solid.

M/Z (M[³⁵Cl]+H)⁺: 304.2.

### Compound 263: 2′-bromo-5′-(4-chloro-3-fluorophenyl)-5′,6′-dihydrospiro[cyclopentane-1,7′-pyrrolo[2,3-b]pyrazine]

Compound 263 (137 mg, 64%) was obtained as a beige solid following general procedure (XVI) in EtOAc, after purification by flash chromatography (Interchim® 50 μm, CyHex 100% to CyHex/EtOAc 70:30), starting from Compound 262 (170 mg).

M/Z (M[⁸¹Br][³⁵Cl]+H)⁺: 384.1

### Example 206: methyl 6-(4-(5′-(4-chloro-3-fluorophenyl)-5′,6′-dihydrospiro[cyclopentane-1,7′-pyrrolo[2,3-b]pyrazine]-2′-carbonyl)-3,3-dimethylpiperazin-1-yl)-2,4-dimethylnicotinate

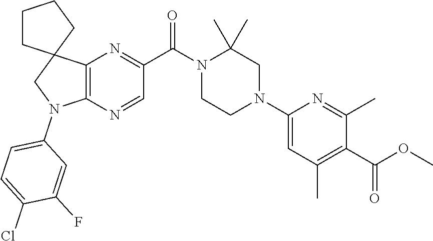 [SMILES: COC(=O)c1c(C)cc(N2CCN(C(=O)c3cnc4c(n3)C3(CCCC3)CN4c3ccc(Cl)c(F)c3)C(C)(C)C2)nc1C]

Example 206 was prepared according to general procedure (XIV) starting from Compound 263 (137 mg) and Compound 10 (1.5 eq), and using triethyl amine (5.0 eq), Mo(CO)₆ (1.95 eq), XantPhos Pd G4 (0.13 eq), and DBU (6 eq). The crude was purified by flash chromatography (Interchim® 50 μm, CyHex 100% to CyHex/EtOAc 40:60) to obtain Example 206 (110 mg) as a yellow solid.

¹H-NMR (DMSO-d₆, 300 MHz) δ: 1.50 (s, 6H, 2CH₃); 1.79-2.03 (m, 8H, 4CH₂); 2.23 (s, 3H, ArCH₃); 2.34 (s, 3H, ArCH₃); 3.52-3.60 (m, 2H, N—CH₂); 3.76-3.83 (m, 5H, N—CH₂O—CH₃); 3.89 (s, 2H, N—CH₂); 4.08 (s, 2H, N—CH₂); 6.36 (m, 1H, Ar); 7.58-7.66 (m, 1H, Ar); 7.76-7.78 (m, 1H, Ar); 8.05-8.07 (m, 1H, Ar); 8.21 (s, 1H, Ar);

M/Z (M[³⁵Cl]+H)⁺: 607.4.

### Example 207: 6-(4-(5′-(4-chloro-3-fluorophenyl)-5′,6′-dihydrospiro[cyclopentane-1,7′-pyrrolo[2,3-b]pyrazine]-2′-carbonyl)-3,3-dimethylpiperazin-1-yl)-2,4-dimethylnicotinic acid

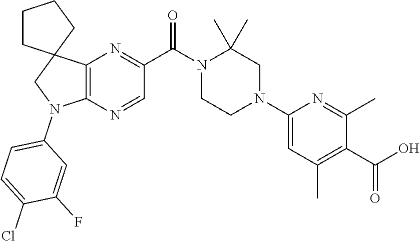 [SMILES: Cc1cc(N2CCN(C(=O)c3cnc4c(n3)C3(CCCC3)CN4c3ccc(Cl)c(F)c3)C(C)(C)C2)nc(C)c1C(=O)O]

Example 207 was prepared according to general procedure (XI) starting from Example 206 (110 mg). The crude was purified by preparative HPLC (Column B, H₂O+0.1% HCOOH/MeCN+0.1% HCOOH 65:35 to 0:100) and freeze dried with HCl (0.1N in water) to obtain Example 207 (35 mg, 16% over 2 steps) as a yellow solid.

¹H-NMR (DMSO-d₆, 300 MHz) δ: 1.49 (s, 6H, 2CH₃); 1.77-2.00 (m, 8H, 4CH₂); 2.27 (s, 3H, ArCH₃); 2.38 (s, 3H, ArCH₃); 3.53-3.60 (m, 2H, N—CH₂); 3.78-3.84 (m, 2H, N—CH₂); 3.88 (s, 2H, N—CH₂); 4.08 (s, 2H, N—CH₂); 6.36 (s, 1H, Ar); 7.62 (t, J 9.0 Hz, 1H, Ar); 7.76 (dd, J 9.0, 2.5 Hz, 1H, Ar); 8.09 (dd, J 12.7, 2.5 Hz, 1H, Ar); 8.22 (s, 1H, Ar); 12.73 (bs, 1H, COOH).

M/Z (M[³⁵Cl]+H)⁺: 593.5.

### Compound 264: 1-(5-chloro-3,3-dimethyl-2,3-dihydro-1H-pyrrolo[3,2-b]pyridin-1-yl)ethan-1-one

To a solution of Compound 70 (500 mg) in DCM (13 mL) was added triethylamine (575 μL) and acetyl chloride (292 μL). The reaction mixture was stirred at 25° C. for 1 hour, then diluted in DCM (20 mL) and washed with K₂CO₃ (sat. aq. 2*10 mL), brine (10 mL) and dried over MgSO₄ to obtain Compound 264 (608 mg, 99%) as a brown solid.

M/Z (M[³⁵Cl]+H)⁺: 225.1.

### Example 208: methyl 6-(4-(1-acetyl-3,3-dimethyl-2,3-dihydro-1H-pyrrolo[3,2-b]pyridine-5-carbonyl)-3,3-dimethylpiperazin-1-yl)-2,4-dimethylnicotinate

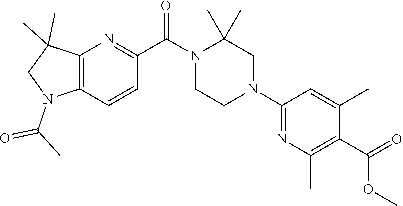 [SMILES: COC(=O)c1c(C)cc(N2CCN(C(=O)c3ccc4c(n3)C(C)(C)CN4C(C)=O)C(C)(C)C2)nc1C]

Example 208 was prepared according to general procedure (XIV) starting from Compound 264 (200 mg) and Compound 10 (1.5 eq), and using triethyl amine (5.0 eq), Mo(CO)₆ (1.5 eq), XantPhos Pd G4 (0.10 eq), and DBU (4.5 eq). The crude was purified by flash chromatography (Interchim® 50 μm, DCM 100% to DCM/MeOH 97:03) to obtain Example 208 (453 mg) as a yellow oil.

¹H-NMR (DMSO-d₆, 300 MHz) δ: 1.34 (s, 6H, 2CH₃); 1.49 (s, 6H, 2CH₃); 2.20 (s, 3H, CH₃—C(O)); 2.23 (s, 3H, ArCH₃); 2.34 (s, 3H, ArCH₃); 3.50-3.55 (m, 2H, N—CH₂); 3.65-3.70 (m, 2H, N—CH₂); 3.75 (s, 3H, O—CH₃); 3.89 (s, 2H, N—CH₂); 3.98 (s, 2H, N—CH₂); 6.36 (m, 1H, Ar); 7.39 (d, J 8.4 Hz, 1H, Ar); 8.27 (d, J 8.4 Hz, 1H, Ar);

M/Z (M+H)⁺: 494.4.

### Compound 265: methyl 6-(4-(3,3-dimethyl-2,3-dihydro-1H-pyrrolo[3,2-b]pyridine-5-carbonyl)-3,3-dimethylpiperazin-1-yl)-2,4-dimethylnicotinate

To a solution of Example 208 (439 mg) in MeOH (20 mL) was added HCl (6N, aq. 15 mL). The reaction mixture was heated at 100° C. for 30 minutes then quenched with NaOH (6N, aq. to pH>7) and extracted with EtOAc (2*50 mL).

The combined organic layers were washed with brine, dried over MgSO4 and concentrated under reduced pressure to obtain Compound 265 (249 mg, 62% over 2 steps) as an orange oil.

M/Z (M+H)⁺: 452.4.

### Example 209: 6-(4-(1-(1,3-dihydroisobenzofuran-5-yl)-3,3-dimethyl-2,3-dihydro-1H-pyrrolo[3,2-b]pyridine-5-carbonyl)-3,3-dimethylpiperazin-1-yl)-2,4-dimethylnicotinic acid

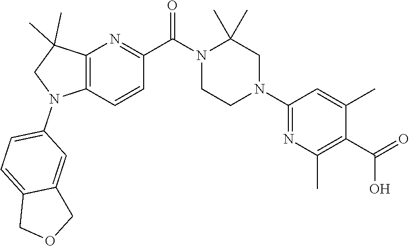 [SMILES: Cc1cc(N2CCN(C(=O)c3ccc4c(n3)C(C)(C)CN4c3ccc4c(c3)COC4)C(C)(C)C2)nc(C)c1C(=O)O]

To a solution of Compound 265 (83 mg) in ^{t}BuOH (2.0 mL) was added Sodium ^{t}Butoxide (177 mg) and the reaction mixture was heated at 60° C. for 24 hours. 5-Bromo-1,3-dihydro-isobenzofuran (73 mg) was added at 25° C., the reaction mixture was sparged with Argon for 10 minutes, RuPhos Pd G4 (8 mg) was added and the reaction mixture was heated at 80° C. for 18 hours. The reaction mixture was cooled at 40° C. and MeOH (1.0 mL) was added, the mixture was filtered, formic acid (104 μL) and DMSO (0.5 mL) were added and the mixture was filtered. The filtrate was purified by preparative HPLC (Column B, H₂O+0.1% HCOOH/MeCN+0.1% HCOOH 75:25 to 35:65) and freeze dried with HCl (0.1N in water) to obtain Example 209 (10 mg, 9%) as a yellow solid.

¹H-NMR (DMSO-d₆, 300 MHz) δ: 1.36 (s, 6H, 2CH₃); 1.52 (s, 6H, 2CH₃); 2.33 (s, 3H, ArCH₃); 3.63-3.70 (m, 2H, N—CH₂); 3.84 (s, 2H, N—CH₂); 3.88-3.94 (m, 4H, 2N—CH₂); 4.98-5.00 (m, 4H, 2O—CH₂); 6.52-6.93 (m, 1H, Ar); 7.19 (dd, J 8.4, 2.0 Hz, 1H, Ar); 7.27 (s, 1H, Ar); 7.31-7.35 (m, 2H, 2Ar); 7.42 (d, J 8.4 Hz, 1H, Ar); One CH₃ and COOH were not observed.

M/Z (M+H)⁺: 556.4

Compound 266 3-chloro-N-(4-fluorophenethyl)pyrazin-2-amine

A solution of 2,3-dichloropyrazine (500 mg) and 4-Fluorophenethylamine (1.05 g) in MeTHF (5 mL) was heated at 110° C. for 16 hours. The reaction mixture was washed with HCl (aq. 0.1N, 50 mL), and extracted with EtOAc (2*50 mL). The combined organic layers were washed with Brine (10 mL), dried over MgSO₄, and concentrated under reduced pressure to obtain crude Compound 266 (857 mg).

M/Z (M[³⁵Cl]+H)⁺: 252.1.

### Compound 267: 3-chloro-N-(4-fluorophenethyl)-N-(2-methylallyl)pyrazin-2-amine

To a solution of Compound 266 (857 mg) in THE (34 mL) was added potassium tert-butoxide (535 mg) at 0° C. After 10 minutes, 3-bromo-2-methylprop-1-ene (708 μL) was added and the reaction mixture was stirred at 25° C. for 48 hours, then heated at 50° C. for 4 hours. Another portion of base (535 mg) and 3-bromo-2-methylprop-1-ene (708 μL) were added and the mixture was heated at 50° C. for another 17 hours. The reaction mixture was quenched with NH₄Cl (sat. aq. 150 mL), diluted with water (50 mL), and extracted with EtOAc (3*150 mL). The combined organic layers were washed with brine (50 mL), dried over MgSO₄, and concentrated under reduced pressure. The residue was purified by flash chromatography (Interchim® 50 μm, CyHex 100% to CyHex/EtOAc 90:10) to obtain Compound 222 (660 mg, 54% over 2 steps) as an orange oil.

M/Z (M[³⁵Cl]+H)⁺: 306.2.

### Compound 268: 5-(4-fluorophenethyl)-7,7-dimethyl-6,7-dihydro-5H-pyrrolo[2,3-b]pyrazine

Compound 268 was prepared according to general procedure (XVIII) starting from Compound 267 (660 mg). The crude was purified by flash chromatography (Interchim® 50 μm, CyHex 100% to CyHex/EtOAc 50:50) to obtain Compound 268 (417 mg, 71%) as a yellow oil.

M/Z (M[³⁵Cl]+H)⁺: 272.2.

### Compound 269: 2-bromo-5-(4-fluorophenethyl)-7,7-dimethyl-6,7-dihydro-5H-pyrrolo[2,3-b]pyrazine

Compound 269 (531 mg, 99%) was obtained without further purification as a yellow oil following general procedure (XVI) in EtOAc, starting from Compound 268 (417 mg).

M/Z (M[³⁵Cl][⁸¹Br]+H)⁺: 350.2.

### Example 210: methyl 2-(6-(4-(5-(4-fluorophenethyl)-7,7-dimethyl-6,7-dihydro-5H-pyrrolo[2,3-b]pyrazine-2-carbonyl)-3,3-dimethylpiperazin-1-yl)pyridin-3-yl)acetate

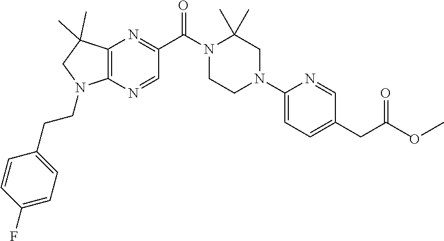

Example 210 was prepared according to general procedure (XIV) starting from Compound 269 (150 mg) and Compound 111 (1.5 eq), and using triethyl amine (4.0 eq), Mo(CO)₆ (1.5 eq), XantPhos Pd G4 (0.1 eq), and DBU (4.5 eq). The crude was purified by flash chromatography (Interchim® 50 μm, CyHex 100% to CyHex/EtOAc 00:100) to obtain Example 210 (54 mg, 23%) as a yellow solid.

¹H-NMR (DMSO-d₆, 300 MHz) δ: 1.20 (s, 6H, 2CH₃); 1.46 (s, 6H, 2CH₃); 2.91 (t, J 7.2 Hz, 2H, Ar—CH₂); 3.39 (s, 2H, N—CH₂); 3.46-3.55 (m, 4H, N—CH₂N—CH₂); 3.60-3.64 (m, 5H, N—CH₂O—CH₃); 3.76-3.81 (m, 4H, 2N—CH₂); 6.58 (d, J 8.7 Hz, 1H, Ar); 7.08-7.14 (m, 2H, 2Ar); 7.25-7.31 (m, 2H, 2Ar); 7.41-7.45 (m, 1H, Ar); 7.93-8.00 (m, 2H, 2Ar);

M/Z (M+H)⁺: 561.5

### Example 211: 2-(6-(4-(5-(4-fluorophenethyl)-7,7-dimethyl-6,7-dihydro-5H-pyrrolo[2,3-b]pyrazine-2-carbonyl)-3,3-dimethylpiperazin-1-yl)pyridin-3-yl)acetic acid

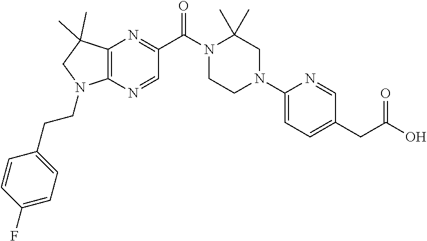 [SMILES: CC1(C)CN(CCc2ccc(F)cc2)c2ncc(C(=O)N3CCN(c4ccc(CC(=O)O)cn4)CC3(C)C)nc21]

Example 211 was prepared according to general procedure (VII) starting from Example 210 (54 mg). The crude was purified by preparative HPLC (Column B, H₂O+0.1% HCOOH/MeCN+0.1% HCOOH 80:20 to 40:60) and freeze dried with HCl (0.1N in water) to obtain Example 211 (35 mg, 62%) as a yellow solid.

¹H-NMR (DMSO-d₆, 300 MHz) δ: 1.23 (s, 6H, 2CH₃); 1.52 (s, 6H, 2CH₃); 2.91 (t, J 7.2 Hz, 2H, Ar—CH₂); 3.39 (s, 2H, CH₂C(O)); 3.62-3.70 (m, 4H, N—CH₂N—CH₂); 3.72 (t, J 5.3 Hz, 2H, N—CH₂); 3.90 (t, J 5.3 Hz, 2H, N—CH₂); 3.98 (s, 2H, N—CH₂); 7.08-7.14 (m, 2H, 2Ar); 7.28-7.34 (m, 2H, 2Ar); 7.37-7.41 (m, 1H, Ar); 7.95-8.00 (m, 3H, 3Ar); COOH was not observed.

M/Z (M+H)⁺: 547.4.

### Example 212: methyl 6-(4-(5-(4-fluorophenethyl)-7,7-dimethyl-6,7-dihydro-5H-pyrrolo[2,3-b]pyrazine-2-carbonyl)-3,3-dimethylpiperazin-1-yl)-2,4-dimethylnicotinate

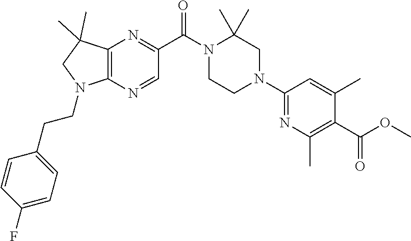 [SMILES: COC(=O)c1c(C)cc(N2CCN(C(=O)c3cnc4c(n3)C(C)(C)CN4CCc3ccc(F)cc3)C(C)(C)C2)nc1C]

Example 212 was prepared according to general procedure (XIV) starting from Compound 269 (150 mg) and Compound 10 (1.5 eq), and using triethyl amine (4.0 eq), Mo(CO)₆ (1.5 eq), XantPhos Pd G4 (0.1 eq), and DBU (4.5 eq). The crude was purified by flash chromatography (Interchim® 50 μm, CyHex 100% to CyHex/EtOAc 50:50) to obtain Example 212 (144 mg, 59%) as a yellow solid.

¹H-NMR (DMSO-d₆, 300 MHz) δ: 1.20 (s, 6H, 2CH₃); 1.46 (s, 6H, 2CH₃); 2.23 (s, 3H, ArCH₃); 2.34 (s, 3H, ArCH₃); 2.90 (t, J 7.2 Hz, 2H, Ar—CH₂); 3.37 (s, 2H, N—CH₂); 3.52-3.55 (m, 2H, N—CH₂); 3.62 (t, J 7.2 Hz, 2H, N—CH₂); 3.76-3.86 (m, 5H, N—CH₂O—CH₃); 3.99 (s, 2H, N—CH₂); 6.35 (s, 1H, Ar); 7.07-7.14 (m, 2H, 2Ar); 7.27-7.31 (m, 2H, 2 Ar); 7.93 (s, 1H, Ar);

M/Z (M+H)⁺: 575.5

### Example 213: 2-(6-(4-(5-(4-fluorophenethyl)-7,7-dimethyl-6,7-dihydro-5H-pyrrolo[2,3-b]pyrazine-2-carbonyl)-3,3-dimethylpiperazin-1-yl)pyridin-3-yl)acetic acid

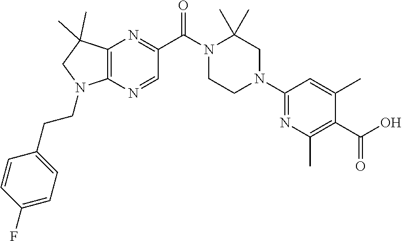 [SMILES: Cc1cc(N2CCN(C(=O)c3cnc4c(n3)C(C)(C)CN4CCc3ccc(F)cc3)C(C)(C)C2)nc(C)c1C(=O)O]

Example 213 was prepared according to general procedure (XI) starting from Example 212 (144 mg). The crude was purified by preparative HPLC (Column B, H₂O+0.1% HCOOH/MeCN+0.1% HCOOH 95:05 to 0:100) and freeze dried with HCl (0.1N in water) to obtain Example 213 (97 mg, 65%) as a beige solid.

¹H-NMR (DMSO-d₆, 300 MHz) δ: 1.23 (s, 6H, 2CH₃); 1.51 (s, 6H, 2CH₃); 2.36 (s, 3H, ArCH₃); 2.55 (s, 3H, ArCH₃); 2.91 (t, J 6.8 Hz, 2H, Ar—CH₂); 3.39 (s, 2H, N—CH₂); 3.64 (t, J 6.8 Hz, 2H, N—CH₂); 3.71-3.74 (m, 2H, N—CH₂); 3.86-3.89 (m, 2H, N—CH₂); 3.96 (s, 2H, N—CH₂); 6.91 (s, 1H, Ar); 7.07-7.13 (m, 2H, 2Ar); 7.28-7.33 (m, 2H, 2 Ar); 7.95 (s, 1H, Ar); COOH was not observed.

M/Z (M+H)⁺: 561.5.

### Compound 270: 1-(3-chloropyridin-2-yl)-3,3-dimethylcyclobutane-1-carbonitrile

Compound 270 was prepared according to general procedure (I) from 3-chloro-2-fluoropyridine (820 mg) and 3,3-dimethylcyclobutane-1-carbonitrile. The crude was purified by flash chromatography (Interchim® 50 μm, CyHex 100% to CyHex/EtOAc 85:15) to obtain Compound 270 (886 mg, 64%) as a yellow oil.

M/Z (M[³⁵Cl]+H⁺): 221.6

### Compound 271: (1-(3-chloropyridin-2-yl)-3,3-dimethylcyclobutyl)methanamine

Compound 271 (440 mg, 49%) was obtained as a white solid following general procedure (XVa), starting from Compound 270 (886 mg).

M/Z (M[³⁵Cl]+H⁺): 225.2.

### Compound 272: 1′-(4-chloro-3-fluorophenyl)-3,3-dimethyl-1′,2′-dihydrospiro[cyclobutane-1,3′-pyrrolo[32-b]pyridine]

Compound 272 was prepared according to general procedure (Xc) from Compound 271 (440 mg) and 4-bromo-1-chloro-2-fluorobenzene. The crude was purified by flash chromatography (Interchim® 50 μm, CyHex 100% to CyHex/EtOAc 90:10) to obtain Compound 272 (197 mg, 32%) as a white solid.

M/Z (M[³⁵Cl]+H⁺): 317.3.

### Compound 273: 5′-bromo-1′-(4-chloro-3-fluorophenyl)-3,3-dimethyl-1′,2′-dihydrospiro[cyclobutane-1,3′-pyrrolo[3,2-b]pyridine]

Compound 273 (230 mg, 97%) was obtained as a beige solid according to general procedure (XVI) in EtOAc from Compound 272 (197 mg) without further purification.

M/Z (M[³⁵Cl][⁸¹Br]+H)⁺: 397.1.

### Example 214: methyl 6-(4-(1′-(4-chloro-3-fluorophenyl)-3,3-dimethyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinate

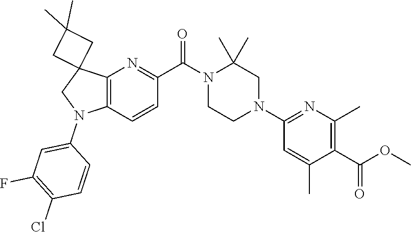 [SMILES: COC(=O)c1c(C)cc(N2CCN(C(=O)c3ccc4c(n3)C3(CN4c4ccc(Cl)c(F)c4)CC(C)(C)C3)C(C)(C)C2)nc1C]

Example 214 was prepared according to general procedure (XIV) starting from Compound 273 (230 mg) and Compound 10 (2.5 eq), and using triethyl amine (6.1 eq), Mo(CO)₆ (2.6 eq), XantPhos Pd G4 (0.18 eq), and DBU (6.9 eq). The crude was purified by flash chromatography (Interchim® 50 μm, CyHex 100% to CyHex/EtOAc 85:15) to obtain Example 214 (140 mg, 39%) as a white solid.

¹H-NMR (DMSO-d₆, 300 MHz) δ: 1.28-1.30 (m, 6H, 2CH₃); 1.51 (s, 6H, 2CH₃); 2.05-2.10 (m, 2H, cyclobutyl); 2.23 (s, 3H, ArCH₃); 2.25-2.29 (m, 1H, cyclobutyl); 2.34 (s, 3H, ArCH₃); 2.37-2.39 (m, 1H, cyclobutyl); 3.67-3.70 (m, 2H, N—CH₂); 3.78 (s, 3H, O—CH₃); 3.87-3.94 (m, 4H, 2N—CH₂); 4.29 (s, 2H, N—CH₂); 6.35 (s, 1H, Ar); 7.22 (dd, J 9.0, 2.1 Hz, 1H, Ar); 7.34-7.41 (m, 2H, 2Ar); 7.51-7.58 (m, 2H, 2Ar).

M/Z (M[³⁵Cl]+H)⁺: 620.4

### Example 215: 6-(4-(1′-(4-chloro-3-fluorophenyl)-3,3-dimethyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid

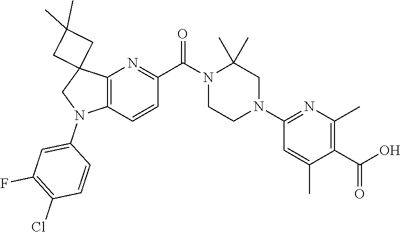 [SMILES: Cc1cc(N2CCN(C(=O)c3ccc4c(n3)C3(CN4c4ccc(Cl)c(F)c4)CC(C)(C)C3)C(C)(C)C2)nc(C)c1C(=O)O]

Example 215 was prepared according to general procedure (XI) starting from Example 214 (140 mg). The crude was purified by preparative HPLC (Column B, H₂O+0.1% HCOOH/MeCN+0.1% HCOOH 65:35 to 0:100) and freeze dried with HCl (0.1N in water) to obtain Example 215 (49 mg, 34% over 2 steps) as a yellow solid.

¹H-NMR (DMSO-d₆, 300 MHz) δ: 1.28-1.29 (m, 6H, 2CH₃); 1.55 (s, 6H, 2CH₃); 2.07 (d, J 12.1 Hz, 2H, cyclobutyl); 2.34-2.38 (m, 5H, cyclobutyl ArCH₃); 2.54 (bs, 3H, ArCH₃); 3.76-3.80 (m, 2H, N—CH₂); 3.98-4.02 (m, 4H, 2N—CH₂); 4.28 (s, 2H, N—CH₂); 6.73 (s, 1H, Ar); 7.22 (dd, J 9.0, 2.1 Hz, 1H, Ar); 7.34-7.42 (m, 2H, 2Ar); 7.51-7.57 (m, 2H, 2Ar); COOH was not observed.

M/Z (M[³⁵Cl]+H)⁺: 606.3.

### Compound 274: 3-chloro-N-(2-methylallyl)-N-(3,4,5-trifluorophenyl)pyrazin-2-amine

Crude Compound 274 (1220 mg) was obtained as a brown oil according to general procedure (XVII) on a 500 mg scale, using 3,4,5-trifluoroaniline.

M/Z (M[³⁵Cl]+H)⁺: 314.2.

### Compound 275: 7,7-dimethyl-5-(3,4,5-trifluorophenyl)-6,7-dihydro-5H-pyrrolo[2,3-b]pyrazine

Compound 275 was prepared according to general procedure (XVIII) starting from crude Compound 274 (970 mg). The crude was purified by flash chromatography (Interchim® 50 μm, CyHex 100% to CyHex/EtOAc 50:50) to obtain Compound 275 (461 mg, 49% over 2 steps) as a yellow oil.

M/Z (M+H)⁺: 280.1

### Compound 276: 2-bromo-7,7-dimethyl-5-(3,4,5-trifluorophenyl)-6,7-dihydro-5H-pyrrolo[2,3-b]pyrazine

Compound 276 (566 mg, 96%) was obtained as a brown solid following general procedure (XVI) in EtOAc without further purification, starting from Compound 275 (461 mg).

M/Z (M[⁷⁹Br]+H)⁺: 358.1.

### Example 216: methyl 2-(6-(4-(7,7-dimethyl-5-(3,4,5-trifluorophenyl)-6,7-dihydro-5H-pyrrolo[2,3-b]pyrazine-2-carbonyl)-3,3-dimethylpiperazin-1-yl)pyridin-3-yl)acetate

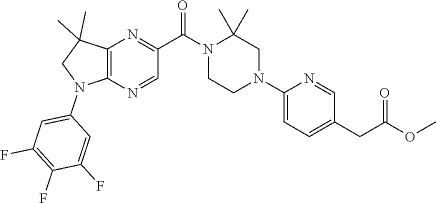 [SMILES: COC(=O)Cc1ccc(N2CCN(C(=O)c3cnc4c(n3)C(C)(C)CN4c3cc(F)c(F)c(F)c3)C(C)(C)C2)nc1]

Example 216 was prepared according to general procedure (XIV) starting from Compound 276 (150 mg) and Compound 111 (1.5 eq), and using triethyl amine (3.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 50:50) to obtain Example 216 (160 mg, 67%) as a green oil.

¹H-NMR (DMSO-d₆, 300 MHz) δ: 1.39 (s, 6H, 2CH₃); 1.50 (s, 6H, 2CH₃); 3.33-3.54 (m, 4H, N—CH₂+CH₂C(O)); 3.60 (s, 3H, O—CH₃); 3.77-3.86 (m, 4H, 2N—CH₂); 3.97 (s, 2H, N—CH₂); 6.58-6.61 (m, 1H, Ar); 7.42-7.46 (m, 1H, Ar); 7.87- 7.93 (m, 2H, 2Ar); 7.95-7.97 (m, 1H, Ar); 8.23 (s, 1H, Ar).

M/Z (M+H)⁺: 569.4

### Example 217: 2-(6-(4-(7,7-dimethyl-5-(3,4,5-trifluorophenyl)-6,7-dihydro-5H-pyrrolo[2,3-b]pyrazine-2-carbonyl)-3,3-dimethylpiperazin-1-yl)pyridin-3-yl)acetic acid

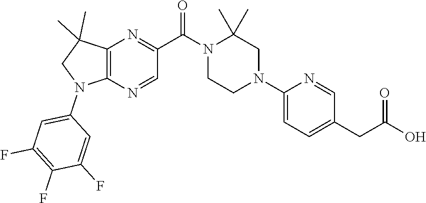 [SMILES: CC1(C)CN(c2cc(F)c(F)c(F)c2)c2ncc(C(=O)N3CCN(c4ccc(CC(=O)O)cn4)CC3(C)C)nc21]

Example 217 was prepared according to general procedure (VII) starting from Example 216 (60 mg). The crude was purified by preparative HPLC (Column B, H₂O+0.1% HCOOH/MeCN+0.1% HCOOH 65:35 to 0:100) and freeze dried with HCl (0.1N in water) to obtain Example 217 (28 mg, 22% over 2 steps) as a white solid.

¹H-NMR (DMSO-d₆, 300 MHz) δ: 1.40 (s, 6H, 2CH₃); 1.56 (s, 6H, 2CH₃); 3.65 (s, 2H, N—CH₂); 3.72 (t, J 5.2 Hz, 2H, N—CH₂); 3.91 (t, J 5.2 Hz, 2H, N—CH₂); 3.97-3.98 (m, 4H, Ar—CH₂+N—CH₂); 7.33-7.36 (m, 1H, Ar); 7.88-7.97 (m, 4H, 4Ar); 8.26 (s, 1H, Ar); COOH

M/Z (M+H)⁺: 555.4.

### Example 218: methyl 2-(6-(4-(5-(3-chloro-4-(trifluoromethyl)phenyl)-7,7-dimethyl-6,7-dihydro-5H-pyrrolo[2,3-b]pyrazine-2-carbonyl)-3,3-dimethylpiperazin-1-yl)pyridin-3-yl)acetate

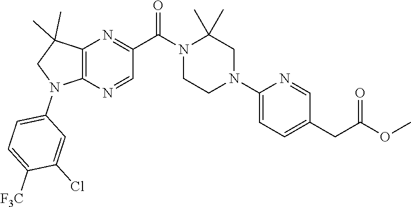 [SMILES: COC(=O)Cc1ccc(N2CCN(C(=O)c3cnc4c(n3)C(C)(C)CN4c3ccc(C(F)(F)F)c(Cl)c3)C(C)(C)C2)nc1]

Example 218 was prepared according to general procedure (XIV) starting from Compound 239 (150 mg) and Compound 111 (1.5 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 50:50) to obtain Example 218 (157 mg, 67%) as a yellow solid.

¹H-NMR (DMSO-d₆, 300 MHz) δ: 1.40 (s, 6H, 2CH₃); 1.50 (s, 6H, 2CH₃); 3.46-3.54 (m, 4H, N—CH₂+CH₂C(O)); 3.60 (s, 3H, O—CH₃); 3.77-3.85 (m, 4H, 2N—CH₂); 3.99 (s, 2H, N—CH₂); 6.59 (d, J 9 Hz, 1H, Ar); 7.42-7.46 (m, 1H, Ar); 7.86-7.89 (m, 1H, Ar); 7.95-7.97 (m, 2H, 2Ar); 8.27 (s, 1H, Ar); 8.30-8.31 (m, 1H, Ar).

M/Z (M[³⁵Cl]+H)⁺: 617.4

### Example 219: 2-(6-(4-(5-(3-chloro-4-(trifluoromethyl)phenyl)-7,7-dimethyl-6,7-dihydro-5H-pyrrolo[2,3-b]pyrazine-2-carbonyl)-3,3-dimethylpiperazin-1-yl)pyridin-3-yl)acetic acid

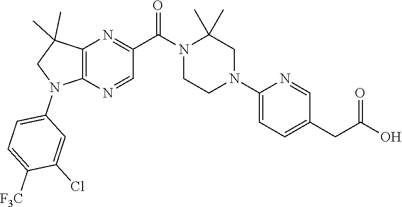 [SMILES: CC1(C)CN(c2ccc(C(F)(F)F)c(Cl)c2)c2ncc(C(=O)N3CCN(c4ccc(CC(=O)O)cn4)CC3(C)C)nc21]

Example 219 was prepared according to general procedure (VII) starting from Example 216 (140 mg). The crude was purified by preparative HPLC (Column A, H₂O+0.1% HCOOH/MeCN+0.1% HCOOH 80:20 to 60:40) and freeze dried with HCl (0.1N in water) to obtain Example 217 (20 mg, 8% over 2 steps) as a yellow solid.

¹H-NMR (DMSO-d₆, 300 MHz) δ: 1.41 (s, 6H, (CH₃)₂); 1.55 (s, 6H, (CH₃)₂); 3.57-3.61 (m, 2H, N—CH₂); 3.64-3.68 (m, 2H, N—CH₂); 3.85-3.89 (m, 2H, N—CH₂); 3.92 (s, 2H, CH₂C(O)); 4.05 (s, 2H, N—CH₂); 7.22 (bs, 1H, Ar); 7.79-7.89 (m, 2H, 2Ar); 7.94-7.99 (m, 2H, 2Ar); 8.30-8.32 (m, 2H, 2Ar); 12.53 (bs, 1H, COOH).

M/Z (M[³⁵Cl]+H)⁺: 617.4

### Compound 277: 3-chloro-N-(3,4-dichlorophenyl)-N-(2-methylallyl)pyrazin-2-amine

Crude Compound 277 (1110 mg) was obtained as a yellow oil according to general procedure (XVII) on a 500 mg scale, using 3,4-dichloroaniline.

M/Z (M[³⁵Cl₃]+H)⁺: 330.2

### Compound 278: 5-(3,4-dichlorophenyl)-7,7-dimethyl-6,7-dihydro-5H-pyrrolo[2,3-b]pyrazine

Compound 278 was prepared according to general procedure (XVIII) starting from crude Compound 277 (1110 mg). The crude was purified by flash chromatography (Interchim® 50 μm, CyHex 100% to CyHex/EtOAc 60:40) to obtain Compound 278 (505 mg, 52% over 2 steps) as a white solid.

M/Z (M[³⁵Cl₂]+H)⁺: 294.1

### Compound 279: 2-bromo-5-(3,4-dichlorophenyl)-7,7-dimethyl-6,7-dihydro-5H-pyrrolo[2,3-b]pyrazine

Compound 279 (457 mg, 71%) was obtained as a yellow solid following general procedure (XVI) in EtOAc after purification by flash chromatography (Interchim® 50 μm, CyHex 100% to CyHex/EtOAc 70:30), starting from Compound 278 (505 mg).

M/Z (M[³⁵Cl₂][⁷⁹Br]+H)⁺: 372.1

### Example 220: methyl 2-(6-(4-(5-(3,4-dichlorophenyl)-7,7-dimethyl-6,7-dihydro-5H-pyrrolo[2,3-b]pyrazine-2-carbonyl)-3,3-dimethylpiperazin-1-yl)pyridin-3-yl)acetate

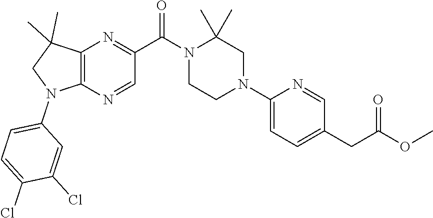 [SMILES: COC(=O)Cc1ccc(N2CCN(C(=O)c3cnc4c(n3)C(C)(C)CN4c3ccc(Cl)c(Cl)c3)C(C)(C)C2)nc1]

Example 220 was prepared according to general procedure (XIV) starting from Compound 279 (150 mg) and Compound 111 (1.5 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 220 (100 mg) as a yellow solid

M/Z (M[³⁵Cl]₂+H)⁺: 583.3

### Example 221: 2-(6-(4-(5-(3,4-dichlorophenyl)-7,7-dimethyl-6,7-dihydro-5H-pyrrolo[2,3-b]pyrazine-2-carbonyl)-3,3-dimethylpiperazin-1-yl)pyridin-3-yl)acetic acid

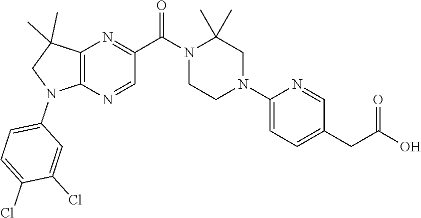 [SMILES: CC1(C)CN(c2ccc(Cl)c(Cl)c2)c2ncc(C(=O)N3CCN(c4ccc(CC(=O)O)cn4)CC3(C)C)nc21]

Example 221 was prepared according to general procedure (VII) starting from Example 220 (100 mg). The crude was purified by preparative HPLC (Column A, H₂O+0.1% HCOOH/MeCN+0.1% HCOOH 80:20 to 60:40) and freeze dried with HCl (0.1N in water) to obtain Example 221 (8 mg, 4% over 2 steps) as a yellow solid.

¹H-NMR (DMSO-d₆, 300 MHz) δ: 1.39 (s, 6H, (CH₃)₂); 1.54 (s, 6H, (CH₃)₂); 3.58-3.62 (m, 2H, N—CH₂); 3.64-3.68 (m, 2H, N—CH₂); 3.87-3.91 (m, 4H, CH₂C(O)N—CH₂); 4.00 (s, 2H, N—CH₂); 7.25 (bs, 1H, Ar); 7.67 (d, J 9.0 Hz, 1H, Ar); 7.85-7.89 (m, 2H, 2Ar); 7.94 (s, 1H, Ar); 8.24 (s, 2H, 2Ar); 12.58 (bs, 1H, COOH)

M/Z (M[³⁵Cl]₂+H)⁺: 569.3

### Compound 280: N-((1-(3-chloropyrazin-2-yl)cyclopentyl)methyl)-3,4-difluoroaniline

Compound 280 was prepared according to general procedure (XXII) from Compound 260 (380 mg) and 3,4-difluoroaniline. The crude was purified by flash chromatography (Interchim® 20 μm, CyHex 100% to CyHex/EtOAc 60:40) to obtain Compound 280 (469 mg) as a clear oil.

M/Z (M[³⁵Cl]+H)⁺: 324.2

### Compound 281: 5′-(3,4-difluorophenyl)-5′,6′-dihydrospiro[cyclopentane-1,7′-pyrrolo[2,3-b]pyrazine]

Compound 281 was prepared according to general procedure (XXIII) from Compound 280 (460 mg). The crude was purified by flash chromatography (Interchim® 20 μm, CyHex 100% to CyHex/EtOAc 80:20) to obtain Compound 281 (301 mg, 58% over 2 steps) as a white solid.

M/Z (M+H)⁺: 288.3

### Compound 282: 2′-bromo-5′-(3,4-difluorophenyl)-5′,6′-dihydrospiro[cyclopentane-1,7′-pyrrolo[2,3-b]pyrazine]

Compound 282 (264 mg, 69%) was obtained as a beige solid following general procedure (XVI) in EtOAc, after purification by flash chromatography (Interchim® 50 μm, CyHex 100% to CyHex/EtOAc 70:30), starting from Compound 281 (302 mg).

M/Z (M[⁸¹Br]+H)⁺: 366.1

### Example 222: methyl 6-(4-(5′-(3,4-difluorophenyl)-5′,6′-dihydrospiro[cyclopentane-1,7′-pyrrolo[2,3-b]pyrazine]-2′-carbonyl)-3,3-dimethylpiperazin-1-yl)-2,4-dimethylnicotinate

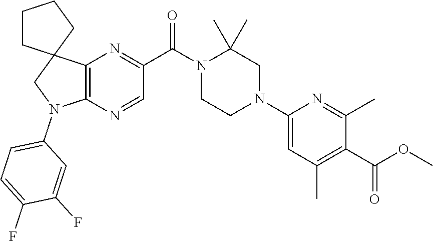 [SMILES: COC(=O)c1c(C)cc(N2CCN(C(=O)c3cnc4c(n3)C3(CCCC3)CN4c3ccc(F)c(F)c3)C(C)(C)C2)nc1C]

Example 222 was prepared according to general procedure (XIV) starting from Compound 282 (150 mg) and Compound 10 (1.5 eq), and using triethyl amine (5.2 eq), Mo(CO)₆ (1.95 eq), XantPhos Pd G4 (0.13 eq), and DBU (6 eq). The crude was purified by flash chromatography (Interchim® 50 μm, CyHex 100% to CyHex/EtOAc 40:60) to obtain Example 222 (178 mg) as a yellow solid.

¹H-NMR (DMSO-d₆, 300 MHz) δ: 1.50 (s, 6H, 2CH₃); 1.79-2.08 (m, 8H, 4CH₂); 2.24 (s, 3H, ArCH₃); 2.35 (s, 3H, ArCH₃); 3.52-3.59 (m, 2H, N—CH₂); 3.76-3.87 (m, 5H, N—CH₂O—CH₃); 3.89 (s, 2H, N—CH₂); 4.08 (s, 2H, N—CH₂); 6.37 (m, 1H, Ar); 7.37-7.59 (m, 1H, Ar); 7.65-7.78 (m, 1H, Ar); 8.05-8.17 (m, 1H, Ar); 8.25 (s, 1H, Ar);

M/Z (M+H)⁺: 591.5.

### Example 223: 6-(4-(5′-(3,4-difluorophenyl)-5′,6′-dihydrospiro[cyclopentane-1,7′-pyrrolo[2,3-b]pyrazine]-2′-carbonyl)-3,3-dimethylpiperazin-1-yl)-2,4-dimethylnicotinic acid

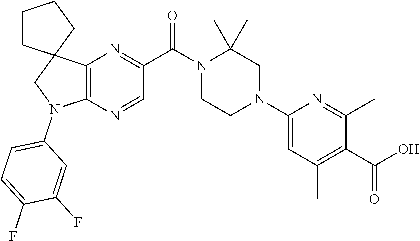 [SMILES: Cc1cc(N2CCN(C(=O)c3cnc4c(n3)C3(CCCC3)CN4c3ccc(F)c(F)c3)C(C)(C)C2)nc(C)c1C(=O)O]

Example 223 was prepared according to general procedure (XI) starting from Example 222 (178 mg). The crude was purified by preparative HPLC (Column B, H₂O+0.1% HCOOH/MeCN+0.1% HCOOH 70:30 to 0:100) and freeze dried with HCl (0.1N in water) to obtain Example 223 (49 mg, 25% over 2 steps) as a white solid.

¹H-NMR (DMSO-d₆, 300 MHz) δ: 1.54 (s, 6H, 2CH₃); 1.77-2.04 (m, 8H, 4CH₂); 2.36 (s, 3H, ArCH₃); 2.54 (s, 3H, ArCH₃); 3.64-3.77 (m, 2H, N—CH₂); 3.85-3.93 (m, 2H, N—CH₂); 3.98 (s, 2H, N—CH₂); 4.07 (s, 2H, N—CH₂); 6.68-7.00 (m, 1H, Ar); 7.45-7.57 (m, 1H, Ar); 7.64-7.74 (m, 1H, Ar); 8.07-8.15 (m, 1H, Ar); 8.20 (s, 1H, Ar); COOH was not observed.

M/Z (M+H)⁺: 577.5.

### Compound 282: (1-(3-chloropyridin-2-yl)-3-methylenecyclobutyl)methanamine

To a solution of Compound 181 (1.35 g) in THE (40 mL) at 0° C. was added a dropwise DIBAL (1N in THF, 29 mL). The reaction mixture was stirred at 0° C. for 2 hours then at 25° C. for 48 hours, and quenched by slow addition in Potassium tartrate (sat. aq. 200 mL). The mixture was extracted with EtOAc (2*100 mL), the combined organic layers were washed with brine (50 mL), dried over MgSO₄ and concentrated under reduced pressure. The residue was purified by Ion Exchange chromatography (Isolute SCX-2, load in DCM, elution with NH₃ 1N in MeOH), to obtain Compound 282 (1.19 g) as an orange oil.

M/Z (M[³⁵Cl]+H⁺): 209.5.

### Compound 283: 1′-(4-chloro-3-fluorophenyl)-3-methylene-1′,2′-dihydrospiro[cyclobutane-1,3′-pyrrolo[3,2-b]pyridine]

Compound 283 was prepared according to general procedure (Xc) from Compound 282 (480 mg) and 4-bromo-1-chloro-2-fluorobenzene. The crude was purified by flash chromatography (Interchim® 50 μm, CyHex 100% to CyHex/EtOAc 70:30) to obtain Compound 283 (314 mg, 459%) as a white solid.

M/Z (M[³⁵Cl]+H⁺): 301.1.

### Compound 284: (1′-(4-chloro-3-fluorophenyl)-1′,2′-dihydrospiro[cyclobutane-1,3′-pyrrolo[3,2-b]pyridin]-3-yl)methanol

To a solution of Compound 283 (403 mg) in THE (16 mL) at 0° C. was added BH₃-DMS (0.51 mL). The reaction mixture was stirred at 0° C. for 1 hour, NaOH (2N aq., 16 mL) was added dropwise at 0° C. followed by H₂O₂ (30% in H₂O, 11 mL) and the mixture was stirred for 18 hours at 25° C. The reaction mixture was diluted in water (50 mL) and extracted with EtOAc (3*100 mL), the combined organic layers were dried over Na₂SO₄ and concentrated under reduced pressure. The residue was dissolved in MeOH (50 mL) treated with HCl (2M in Et₂O, 10 mL) and distilled at 60° C., the distillation was repeated 3 times to recover Compound 284 (472 mg, as a diastereoisomers mixture 80:20), a yellow solid, as the residue,

M/Z (M[³⁵Cl]+H⁺): 319.3.

### Compound 285: 1′-(4-chloro-3-fluorophenyl)-3-(methoxymethyl)-1′,2′-dihydrospiro[cyclobutane-1,3′-pyrrolo[3,2-b]pyridine] major isomer Compound 286: 1′-(4-chloro-3-fluorophenyl)-3-(methoxymethyl)-1′,2′-dihydrospiro[cyclobutane-1,3′-pyrrolo[3,2-b]pyridine] minor isomer

Compound 285 and Compound 286 was prepared according to general procedure (XIII) starting from Compound 284 (472 mg) and Iodomethane (4.0 eq), and using NaH (60% in mineral oil, 4.0 eq) in THF. The crude was purified by flash chromatography (Interchim® 50 μm, CyHex 100% to CyHex/EtOAc 60:40) to obtain Compound 285 (207 mg, 46% over 2 steps) as a white solid and Compound 286 (51 mg, 11% over 2 steps) as a yellow oil.

Compound 285: M/Z (M[³⁵Cl]+H)⁺: 333.3.

Compound 286: M/Z (M[³⁵Cl]+H)⁺: 333.3.

### Compound 287: 5′-bromo-1′-(4-chloro-3-fluorophenyl)-3-(methoxymethyl)-1′,2′-dihydrospiro[cyclobutane-1,3′-pyrrolo[3,2-b]pyridine]major isomer

Compound 287 (247 mg, 63%) was obtained as a clear oil following general procedure (XVI) in EtOAc, after purification by flash chromatography (Interchim® 50 μm, CyHex 100% to CyHex/EtOAc 70:30), starting from Compound 285 (317 mg).

M/Z (M[³⁵Cl][⁸¹Br]+H)⁺: 413.1.

### Example 224: methyl 6-(4-(1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinate major isomer

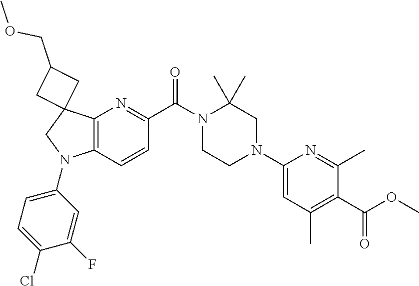

Example 224 was prepared according to general procedure (XIV) starting from Compound 287 (150 mg) and Compound 10 (1.5 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 224 (134 mg) as a yellow solid.

¹H-NMR (DMSO-d₆, 300 MHz) δ: 1.51 (s, 6H, 2×CH₃); 2.17-2.33 (m, 7H, 2CH₂ArCH₃); 2.36 (s, 3H, ArCH₃); 2.60-2.69 (m, 1H, CH); 3.26 (s, 3H, OCH₃), 3.52 (d, J 6.8 Hz, 2H, CH₂—OCH₃); 3.60-3.71 (m, 2H, N—CH₂); 3.79 (s, 3H, C(O)OCH₃); 3.83-3.91 (m, 4H, 2N—CH₂); 4.26 (s, 2H, N—CH₂); 6.37 (s, 1H, Ar); 7.19 (dd, J 8.6, 2.6 Hz, 1H, Ar); 7.34 (dd, J 12.0, 2.6 Hz, 1H, Ar); 7.39 (d, J 8.6 Hz, 1H, Ar); 7.52-7.61 (m, 2H, 2Ar); (M[³⁵Cl]+H)⁺: 636.3.

### Example 225: 6-(4-(1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid major isomer

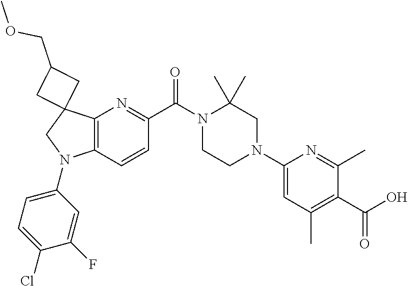 [SMILES: COCC1CC2(C1)CN(c1ccc(Cl)c(F)c1)c1ccc(C(=O)N3CCN(c4cc(C)c(C(=O)O)c(C)n4)CC3(C)C)nc12]

Example 225 was prepared according to general procedure (XI) starting from Example 224 (134 mg). The crude was purified by preparative HPLC (Column B, H₂O+0.1% HCOOH/MeCN+0.1% HCOOH 70:30 to 0:100) and freeze dried with HCl (0.1N in water) to obtain Example 225 (76 mg, 32% over 2 steps) as a yellow solid.

¹H-NMR (DMSO-d₆, 300 MHz) δ: 1.55 (s, 6H, 2CH₃); 2.17-2.33 (m, 4H, 2CH₂); 2.34 (s, 3H, ArCH₃); 2.58-2.69 (m, 1H, CH); 3.26 (s, 3H, OCH₃), 3.51 (d, J 6.8 Hz, 2H, CH₂—OCH₃); 3.69-3.80 (m, 2H, N—CH₂); 3.90-4.04 (m, 2H, N—CH₂); 3.97 (s, 2H, N—CH₂); 4.26 (s, 2H, N—CH₂); 6.61 (s, 1H, Ar); 7.19 (dd, J 8.6, 2.6 Hz, 1H, Ar); 7.34 (dd, J 12.0, 2.6 Hz, 1H, Ar); 7.39 (d, J 8.6 Hz, 1H, Ar); 7.48-7.61 (m, 2H, 2Ar); One CH₃ and COOH were not observed

M/Z (M[³⁵Cl]+H)⁺: 622.4

### Compound 288: 5′-bromo-1′-(4-chloro-3-fluorophenyl)-3-(methoxymethyl)-1′,2′-dihydrospiro[cyclobutane-1,3′-pyrrolo[3,2-b]pyridine]minor isomer

Compound 288 (89 mg, 80%) was obtained as a beige oil following general procedure (XVI) in EtOAc, after purification by flash chromatography (Interchim® 50 μm, CyHex 100% to CyHex/EtOAc 70:30), starting from Compound 286 (90 mg).

M/Z (M[³⁵Cl][⁸¹Br]+H)⁺: 413.1.

### Example 226: methyl 6-(4-(1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinate minor isomer

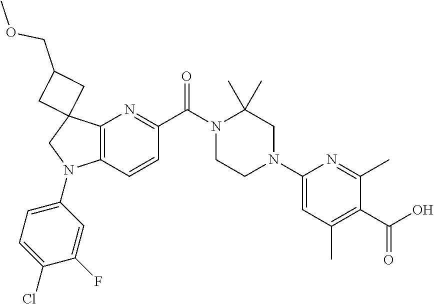 [SMILES: COCC1CC2(C1)CN(c1ccc(Cl)c(F)c1)c1ccc(C(=O)N3CCN(c4cc(C)c(C(=O)O)c(C)n4)CC3(C)C)nc12]

Example 226 was prepared according to general procedure (XIV) starting from Compound 288 (68 mg) and Compound 10 (1.5 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 226 (68 mg) as a yellow solid.

¹H-NMR (DMSO-d₆, 300 MHz) δ: 1.50 (s, 6H, 2CH₃); 2.06-2.17 (m, 2H, 2CHₐH_{b}), 2.19-2.27 (m, 4H, CHₐH_{b} ArCH₃); 2.35 (s, 3H, ArCH₃); 2.37-2.45 (m, 1H, CHₐH_{b}); 2.80-2.92 (m, 1H, CH); 3.29 (s, 3H, OCH₃), 3.42 (d, J 6.3 Hz, 2H, CH₂—OCH₃); 3.60-3.68 (m, 2H, N—CH₂); 3.78 (s, 3H, C(O)OCH₃); 3.84-3.91 (m, 4H, 2N—CH₂); 4.11 (s, 2H, N—CH₂); 6.34 (s, 1H, Ar); 7.18 (dd, J 8.7, 1.5 Hz, 1H, Ar); 7.34 (dd, J 11.7, 2.7 Hz, 1H, Ar); 7.39-7.44 (m, 2H, 2Ar); 7.51-7.58 (m, 2H, 2Ar); (M[³⁵Cl]+H)⁺: 636.3.

### Example 227: 6-(4-(1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid minor isomer

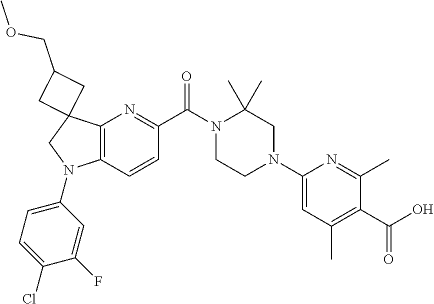 [SMILES: COCC1CC2(C1)CN(c1ccc(Cl)c(F)c1)c1ccc(C(=O)N3CCN(c4cc(C)c(C(=O)O)c(C)n4)CC3(C)C)nc12]

Example 227 was prepared according to general procedure (XI) starting from Example 226 (68 mg). The crude was purified by preparative HPLC (Column B, H₂O+0.1% HCOOH/MeCN+0.1% HCOOH 70:30 to 0:100) and freeze dried with HCl (0.1N in water) to obtain Example 227 (22 mg, 15% over 2 steps) as a white solid.

¹H-NMR (DMSO-d₆, 300 MHz) δ: 1.52 (s, 6H, 2CH₃); 2.05-2.16 (m, 2H, 2CHₐH_{b}); 2.29 (s, 3H, ArCH₃); 2.42 (s, 3H, ArCH₃); 2.80-2.91 (m, 1H, CH); 3.29 (s, 3H, OCH₃), 3.43 (d, J 6.5 Hz, 2H, CH₂O); 3.61-3.70 (m, 2H, N—CH₂); 3.86-3.97 (m, 4H, 2N—CH₂); 4.12 (s, 2H, N—CH₂); 6.27-6.60 (m, 1H, Ar); 7.18 (dd, J 9.0, 2.5 Hz, 1H, Ar); 7.32 (dd, J 11.8, 2.5 Hz, 1H, Ar); 7.40 (d, J 8.4 Hz, 1H, Ar); 7.49-7.60 (m, 2H, 2Ar)

M/Z (M[³⁵Cl]+H)⁺: 622.3

### Example 228: methyl 2-(6-(4-(5′-(3,4-difluorophenyl)-5′,6′-dihydrospiro[cyclopentane-1,7′-pyrrolo[2,3-b]pyrazine]-2′-carbonyl)-3,3-dimethylpiperazin-1-yl)pyridin-3-yl)acetate

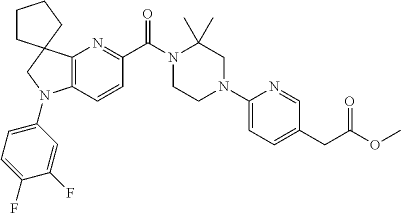 [SMILES: COC(=O)Cc1ccc(N2CCN(C(=O)c3ccc4c(n3)C3(CCCC3)CN4c3ccc(F)c(F)c3)C(C)(C)C2)nc1]

Example 228 was prepared according to general procedure (XIV) starting from Compound 282 (100 mg) and Compound 111 (1.3 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 20:80) to obtain Example 228 (112 mg) as a yellow solid

M/Z (M+H)⁺: 577.4

### Example 229: 2-(6-(4-(5′-(3,4-difluorophenyl)-5′,6′-dihydrospiro[cyclopentane-1,7′-pyrrolo[2,3-b]pyrazine]-2′-carbonyl)-3,3-dimethylpiperazin-1-yl)pyridin-3-yl)acetic acid

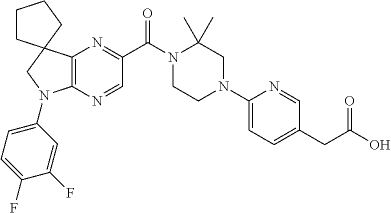 [SMILES: CC1(C)CN(c2ccc(CC(=O)O)cn2)CCN1C(=O)c1cnc2c(n1)C1(CCCC1)CN2c1ccc(F)c(F)c1]

Example 229 was prepared according to general procedure (VII) starting from Example 228 (110 mg). The crude was purified by preparative HPLC (Column A, H₂O+0.1% HCOOH/MeCN+0.1% HCOOH 95:05 to 0:100) and freeze dried with HCl (0.1N in water) to obtain Example 229 (70 mg, 44% over 2 steps) as a white solid.

¹H-NMR (DMSO-d₆, 300 MHz) δ: 1.54 (s, 6H, (CH₃)₂); 1.79-1.91 (m, 8H(CH₂)₄); 3.66 (s, 2H, CH₂C(O)); 3.70-3.73 (m, 2H, N—CH₂); 3.89-3.93 (m, 2H, N—CH₂); 4.00 (s, 2H, N—CH₂); 4.05 (s, 2H, N—CH₂); 7.34-3.37 (m, 1H, Ar); 7.45-7.54 (m, 1H, Ar); 7.66-7.70 (m, 1H, Ar); 7.96-8.00 (m, 2H, 2Ar); 8.06-8.14 (m, 1H, Ar); 8.20 (s, 1H, Ar); 13.97 (bs, 1H, CO₂H).

M/Z (M+H)⁺: 563.4

### Compound 289: methyl 1-(3-chloropyrazin-2-yl)cyclobutane-1-carboxylate

Compound 289 was prepared according to general procedure (Ib) from dichloropyrazine (1.80 g) and methyl cyclobutanecarboxylate. The crude was purified by flash chromatography (Interchim® 20 μm, CyHex 100% to CyHex/EtOAc 70:30) to obtain Compound 289 (1.40 g) as a yellow solid.

M/Z (M[³⁵Cl]+H)⁺: 227.1

### Compound 290: (1-(3-chloropyrazin-2-yl)cyclobutyl)methanol

Compound 290 was prepared according to general procedure (XX) from Compound 289 (1.36 g). The crude was purified by flash chromatography (Interchim® 50 μm, CyHex 100% to CyHex/EtOAc 50:50) to obtain Compound 290 (0.51 g, 22% over 2 steps) as a yellow solid.

M/Z (M[³⁵Cl]+H)⁺: 199.2

### Compound 291: 1-(3-chloropyrazin-2-yl)cyclobutane-1-carbaldehyde

Compound 291 was prepared according to general procedure (XXI) from Compound 290 (505 mg). The crude was purified by flash chromatography (Interchim® 20 μm, CyHex 100% to CyHex/EtOAc 60:40) to obtain Compound 291 (473 mg) as a clear oil.

M/Z (M[³⁵Cl]+H)⁺: 197.1.

### Compound 292: 4-chloro-N-((1-(3-chloropyrazin-2-yl)cyclobutyl)methyl)-3-fluoroaniline

Compound 292 was prepared according to general procedure (XXII) from Compound 291 (450 mg) and 4-chloro-3-fluoroaniline. The crude was purified by flash chromatography (Interchim® 20 μm, CyHex 100% to CyHex/EtOAc 70:30) to obtain Compound 292 (428 mg, 52% over 2 steps) as a white solid.

M/Z (M[³⁵Cl]₂+H)⁺: 326.2.

### Compound 293: 5′-(4-chloro-3-fluorophenyl)-5′,6′-dihydrospiro[cyclobutane-1,7′-pyrrolo[2,3-b]pyrazine]

Compound 293 was prepared according to general procedure (XXIII) from Compound 292 (425 mg). The crude was purified by flash chromatography (Interchim® 20 μm, CyHex 100% to CyHex/EtOAc 70:30) to obtain Compound 293 (344 mg, 91%) as a white solid.

M/Z (M[³⁵Cl]+H)⁺: 290.2.

### Compound 294: 2′-bromo-5′-(4-chloro-3-fluorophenyl)-5′,6′-dihydrospiro[cyclobutane-1,7′-pyrrolo[2,3-b]pyrazine]

Compound 294 (303 mg, 69%) was obtained as a beige solid following general procedure (XVI) in EtOAc, after purification by flash chromatography (Interchim® 50 μm, CyHex 100% to CyHex/EtOAc 70:30), starting from Compound 293 (344 mg).

M/Z (M[⁸¹Br][³⁵Cl]+H)⁺: 380.2.

### Example 230: methyl 2-(6-(4-(5′-(4-chloro-3-fluorophenyl)-5′,6′-dihydrospiro[cyclobutane-1,7′-pyrrolo[2,3-b]pyrazine]-2′-carbonyl)-3,3-dimethylpiperazin-1-yl)pyridin-3-yl)acetate

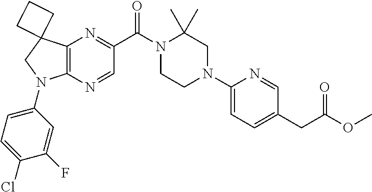 [SMILES: COC(=O)Cc1ccc(N2CCN(C(=O)c3cnc4c(n3)C3(CCC3)CN4c3ccc(Cl)c(F)c3)C(C)(C)C2)nc1]

Example 230 was prepared according to general procedure (XIV) starting from Compound 294 (150 mg) and Compound 111 (1.5 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 230 (180 mg) as a yellow solid.

¹H-NMR (DMSO-d₆, 300 MHz) δ: 1.51 (s, 6H, 2CH₃); 2.00-2.23 (m, 2H, CH₂); 2.24-2.47 (m, 4H, 2C(CH₂); 3.50-3.57 (m, 4H, CH₂C(O)N—CH₂); 3.57-3.66 (m, 5H, O—CH₃N—CH₂); 3.80-3.83 (m, 4H, 2N—CH₂); 4.30 (s, 2H, N—CH₂); 6.53-6.61 (m, 1H, Ar); 7.36-7.48 (m, 1H, 1Ar); 7.58-7.64 (m, 1H, Ar); 7.69-7.73 (m, 1H, Ar); 7.92-7.98 (m, 1H, Ar); 8.04-8.09 (m, 1H, Ar); 8.22 (s, 1H, Ar);

M/Z (M[³⁵Cl]+H)⁺: 579.4.

### Example 231: 2-(6-(4-(5′-(4-chloro-3-fluorophenyl)-5′,6′-dihydrospiro[cyclobutane-1,7′-pyrrolo[2,3-b]pyrazine]-2′-carbonyl)-3,3-dimethylpiperazin-1-yl)pyridin-3-yl)acetic acid

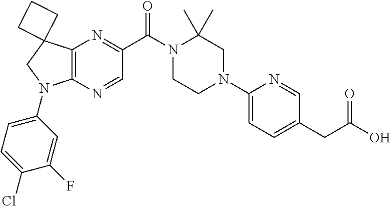 [SMILES: CC1(C)CN(c2ccc(CC(=O)O)cn2)CCN1C(=O)c1cnc2c(n1)C1(CCC1)CN2c1ccc(Cl)c(F)c1]

Example 231 was prepared according to general procedure (VII) starting from Example 230 (180 mg). The crude was purified by preparative HPLC (Column A, H₂O+0.1% HCOOH/MeCN+0.1% HCOOH 65:35 to 0:100) and freeze dried with HCl (0.1N in water) to obtain Example 230 (78 mg, 32% over 2 steps) as a white solid.

¹H-NMR (DMSO-d₆, 300 MHz) δ: 1.58 (s, 6H, 2CH₃); 1.97-2.25 (m, 2H, CH₂); 2.25-2.40 (m, 2H, C(CH₂)); 3.63 (s, 2H, CH₂C(O)); 3.66-3.77 (m, 2H, N—CH₂); 3.93-4.04 (m, 4H, 2N—CH₂); 4.32 (s, 2H, N—CH₂); 7.24 (bs, 1H, Ar); 7.63 (m, 1H, Ar); 7.73 (dd, J 9.0, 2.5 Hz, 1H, Ar); 7.82-8.00 (m, 2H, 2Ar); 8.08 (dd, 12.5, 2.5 Hz, 1H, Ar); 8.25 (s, 1H, Ar); one C(CH₂) and COOH were not observed.

M/Z (M[³⁵Cl]+H)⁺: 565.4

### Compound 295: methyl 1-(3-chloropyrazin-2-yl)-3,3-dimethylcyclobutane-1-carboxylate

Compound 295 was prepared according to general procedure (Ib) from dichloropyrazine (1.85 g) and methyl 3,3-dimethylcyclobutane-1-carboxylate. The crude was purified by flash chromatography (Interchim® 20 μm, CyHex 100% to CyHex/EtOAc 85:15) to obtain Compound 295 (1.40 g, 44%) as a yellow oil.

M/Z (M[³⁵Cl]+H)⁺: 255.1

### Compound 296: (1-(3-chloropyrazin-2-yl)-3,3-dimethylcyclobutyl)methanol

Compound 296 was prepared according to general procedure (XX) from Compound 295 (1.73 g). The crude was purified by flash chromatography (Interchim® 50 μm, CyHex 100% to CyHex/EtOAc 50:50) to obtain Compound 296 (1.00 g, 65%) as a yellow oil.

M/Z (M[³⁵Cl]+H)⁺: 227.1

### Compound 297: 1-(3-chloropyrazin-2-yl)-3,3-dimethylcyclobutane-1-carbaldehyde

Compound 297 was prepared according to general procedure (XXI) from Compound 296 (1000 mg). The crude was purified by flash chromatography (Interchim® 20 μm, CyHex 100% to CyHex/EtOAc 60:40) to obtain Compound 297 (873 mg, 88%) as a yellow oil.

M/Z (M[³⁵Cl]+H)⁺: 225.1

### Compound 298: N-((1-(3-chloropyrazin-2-yl)-3,3-dimethylcyclobutyl)methyl)-3,4-difluoroaniline

Compound 298 was prepared according to general procedure (XXII) from Compound 297 (290 mg) and 3,4-difluoroaniline. The crude was purified by flash chromatography (Interchim® 20 μm, CyHex 100% to CyHex/EtOAc 70:30) to obtain Compound 298 (217 mg, 50%) as a white solid.

M/Z (M[³⁵Cl]+H)⁺: 338.2.

### Compound 299: 5′-(3,4-difluorophenyl)-3,3-dimethyl-5′,6′-dihydrospiro[cyclobutane-1,7′-pyrrolo[2,3-b]pyrazine]

Compound 299 was prepared according to general procedure (XXIII) from Compound 298 (215 mg). The crude was purified by flash chromatography (Interchim® 20 μm, CyHex 100% to CyHex/EtOAc 80:20) to obtain Compound 299 (157 mg, 82%) as a white solid.

M/Z (M+H)⁺: 302.3

### Compound 300: 2′-bromo-5′-(3,4-difluorophenyl)-3,3-dimethyl-5′,6′-dihydrospiro[cyclobutane-1,7′-pyrrolo[2,3-b]pyrazine]

Compound 300 (163 mg, 82%) was obtained as a beige solid following general procedure (XVI) in EtOAc, after purification by flash chromatography (Interchim® 50 μm, CyHex 100% to CyHex/EtOAc 70:30), starting from Compound 299 (157 mg).

M/Z (M[⁸¹Br]+H)⁺: 382.2.

### Example 232: methyl 6-(4-(5′-(3,4-difluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-2,4-dimethylnicotinate

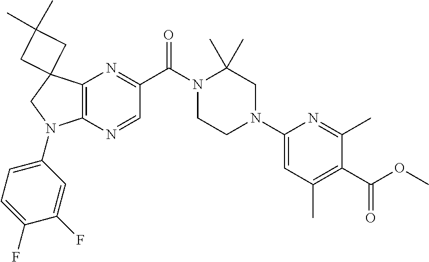 [SMILES: COC(=O)c1c(C)cc(N2CCN(C(=O)c3cnc4c(n3)C3(CN4c4ccc(F)c(F)c4)CC(C)(C)C3)C(C)(C)C2)nc1C]

Example 232 was prepared according to general procedure (XIV) starting from Compound 300 (157 mg) and Compound 10 (1.5 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 232 (131 mg) as a yellow solid.

¹H-NMR (DMSO-d₆, 300 MHz) δ: 1.29 (s, 6H, 2C(CH₃)); 1.51 (s, 6H, C(CH₃)₂); 2.11-2.15 (m, 2H, 2CHₐH_{b}); 2.23 (s, 3H, ArCH₃); 2.34-2.38 (m, 5H, 2CHₐH_{b}ArCH₃); 3.66-3.69 (m, 2H, N—CH₂); 3.78 (s, 3H, OCH₃); 3.86-3.90 (m, 4H, 2N—CH₂); 4.40 (s, 2H, N—CH₂); 6.36 (s, 1H, Ar); 7.45-7.55 (m, 1H, Ar); 7.73-7.75 (m, 1H, Ar); 8.08-8.20 (m, 1H, Ar); 8.53 (s, 1H, Ar).

M/Z (M+H)⁺: 605.4

### Example 233: 6-(4-(5′-(3,4-difluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-2,4-dimethylnicotinic acid

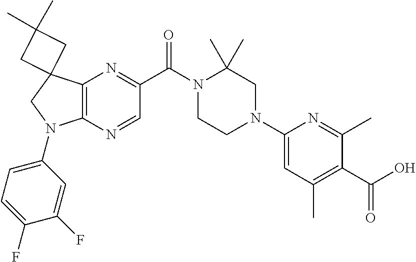 [SMILES: Cc1cc(N2CCN(C(=O)c3cnc4c(n3)C3(CN4c4ccc(F)c(F)c4)CC(C)(C)C3)C(C)(C)C2)nc(C)c1C(=O)O]

Example 233 was prepared according to general procedure (XI) starting from Example 232 (138 mg). The crude was purified by preparative HPLC (Column A, H₂O+0.1% HCOOH/MeCN+0.1% HCOOH 60:40 to 20:80) and freeze dried with HCl (0.1N in water) to obtain Example 233 (54 mg, 21% over 2 steps) as a white solid.

¹H-NMR (DMSO-d₆, 300 MHz) δ: 1.31 (s, 6H, 2C(CH₃)); 1.51 (s, 6H, C(CH₃)₂); 2.11-2.16 (m, 2H, 2CHₐH_{b}); 2.26 (s, 3H, ArCH₃); 2.34-2.38 (m, 5H, 2CHₐH_{b}ArCH₃); 3.65-3.68 (m, 2H, N—CH₂); 3.86-3.89 (m, 4H, 2N—CH₂); 4.41 (s, 2H, N—CH₂); 6.33 (s, 1H, Ar); 7.45-7.55 (m, 1H, Ar); 7.71-7.74 (m, 1H, Ar); 8.08-8.16 (m, 1H, Ar); 8.20 (s, 1H, Ar); 12.69 (bs, 1H, COOH).

M/Z (M+H)⁺: 591.5

### Compound 301: N-((1-(3-chloropyrazin-2-yl)-3,3-dimethylcyclobutyl)methyl)-3,4,5-trifluoroaniline

Compound 301 was prepared according to general procedure (XXII) from Compound 297 (290 mg) and 3,4,5-trifluoroaniline. The crude was purified by flash chromatography (Interchim® 20 μm, CyHex 100% to CyHex/EtOAc 70:30) to obtain Compound 301 (154 mg, 34%) as a clear oil.

M/Z (M[³⁵Cl]+H)⁺: 356.2.

### Compound 302: 3,3-dimethyl-5′-(3,4,5-trifluorophenyl)-5′,6′-dihydrospiro[cyclobutane-1,7′-pyrrolo[2,3-b]pyrazine]

Compound 302 was prepared according to general procedure (XXIII) from Compound 301 (150 mg). The crude was purified by flash chromatography (Interchim® 20 μm, CyHex 100% to CyHex/EtOAc 80:20) to obtain Compound 302 (128 mg, 95%) as a white solid.

M/Z (M+H)⁺: 320.3

### Compound 303: 2′-bromo-3,3-dimethyl-5′-(3,4,5-trifluorophenyl)-5′,6′-dihydrospiro[cyclobutane-1,7′-pyrrolo[2,3-b]pyrazine]

Compound 303 (138 mg, 87%) was obtained as a white solid following general procedure (XVI) in EtOAc, after purification by flash chromatography (Interchim® 50 μm, CyHex 100% to CyHex/EtOAc 70:30), starting from Compound 302 (128 mg).

M/Z (M[⁸¹Br]+H)⁺: 400.2.

### Example 234: methyl 6-(4-(3,3-dimethyl-5′-(3,4,5-trifluorophenyl)-5′,6′-dihydrospiro[cyclobutane-1,7′-pyrrolo[2,3-b]pyrazine]-2′-carbonyl)-3,3-dimethylpiperazin-1-yl)-2,4-dimethylnicotinate

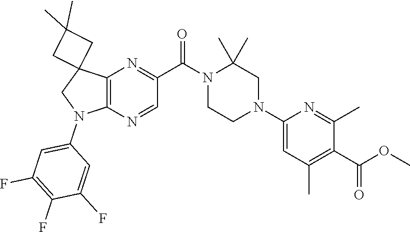 [SMILES: COC(=O)c1c(C)cc(N2CCN(C(=O)c3cnc4c(n3)C3(CN4c4cc(F)c(F)c(F)c4)CC(C)(C)C3)C(C)(C)C2)nc1C]

Example 234 was prepared according to general procedure (XIV) starting from Compound 303 (138 mg) and Compound 10 (1.5 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 234 (111 mg) as a yellow solid.

¹H-NMR (DMSO-d₆, 300 MHz) δ: 1.29-1.31 (m, 6H, 2C(CH₃)); 1.51 (s, 6H, C(CH₃)₂); 2.11-2.15 (m, 2H, 2 CHₐH_{b}); 2.23 (s, 3H, ArCH₃); 2.34-2.38 (m, 5H, 2CHₐH_{b}ArCH₃); 3.65-3.69 (m, 2H, N—CH₂); 3.78 (s, 3H, OCH₃); 3.86-3.90 (m, 4H, 2N—CH₂); 4.40 (s, 2H, N—CH₂); 6.36 (s, 1H, Ar); 7.92-8.01 (m, 2H, 2Ar); 8.53 (s, 1H, Ar).

M/Z (M+H)⁺: 623.4.

### Example 235: 6-(4-(3,3-dimethyl-5′-(3,4,5-trifluorophenyl)-5′,6′-dihydrospiro[cyclobutane-1,7′-pyrrolo[2,3-b]pyrazine]-2′-carbonyl)-3,3-dimethylpiperazin-1-yl)-2,4-dimethylnicotinic acid

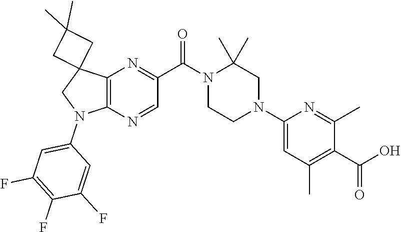 [SMILES: Cc1cc(N2CCN(C(=O)c3cnc4c(n3)C3(CN4c4cc(F)c(F)c(F)c4)CC(C)(C)C3)C(C)(C)C2)nc(C)c1C(=O)O]

Example 235 was prepared according to general procedure (XI) starting from Example 234 (111 mg). The crude was purified by preparative HPLC (Column B, H₂O+0.1% HCOOH/MeCN+0.1% HCOOH 50:50 to 10:90) and freeze dried with HCl (0.1N in water) to obtain Example 235 (48 mg, 21% over 2 steps) as a white solid.

¹H-NMR (DMSO-d₆, 300 MHz) δ: 1.29-1.31 (m, 6H, 2C(CH₃)); 1.52 (s, 6H, C(CH₃)₂); 2.11-2.17 (m, 2H, 2 CHₐH_{b}); 2.28 (s, 3H, ArCH₃); 2.34-2.38 (m, 3H, 2CHₐH_{b}); 2.42 (s, 3H, ArCH₃); 3.65-3.75 (m, 2H, N—CH₂); 3.92-3.97 (m, 4H, 2N—CH₂); 4.40 (s, 2H, N—CH₂); 6.51 (s, 1H, Ar); 7.92-7.98 (m, 2H, 2Ar); 8.08-8.16 (m, 1H, Ar); 8.24 (s, 1H, Ar); COOH was not observed.

M/Z (M+H)⁺: 609.4

### Example 236: methyl 6-(4-(5′-(4-chloro-3-fluorophenyl)-5′,6′-dihydrospiro[cyclobutane-1,7′-pyrrolo[2,3-b]pyrazine]-2′-carbonyl)-3,3-dimethylpiperazin-1-yl)-2,4-dimethylnicotinate

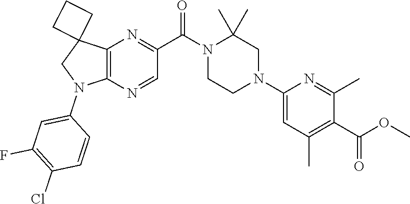 [SMILES: COC(=O)c1c(C)cc(N2CCN(C(=O)c3cnc4c(n3)C3(CCC3)CN4c3ccc(Cl)c(F)c3)C(C)(C)C2)nc1C]

Example 236 was prepared according to general procedure (XIV) starting from Compound 294 (150 mg) and Compound 10 (1.5 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 236 (178 mg) as a yellow solid.

¹H-NMR (DMSO-d₆, 300 MHz) δ: 1.51 (s, 6H, C(CH₃)₂); 1.99-2.21 (m, 2H, 2CHₐH_{b}); 2.23 (s, 3H, ArCH₃); 2.26-2.36 (m, 5H, 2CHₐH_{b}ArCH₃); 3.58-3.68 (m, 2H, CH₂N—CH₂); 3.78 (s, 3H, O—CH₃); 3.82-3.91 (m, 4H, 2N—CH₂); 4.31 (s, 2H, N—CH₂); 6.38 (s, 1H, Ar); 7.59-7.65 (m, 1H, Ar); 7.71-7.74 (m, 1H, Ar); 8.05-8.10 (m, 1H, Ar); 8.23 (s, 1H, Ar);

M/Z (M[³⁵Cl]+H)⁺: 593.5

### Example 237: 6-(4-(5′-(4-chloro-3-fluorophenyl)-5′,6′-dihydrospiro[cyclobutane-1,7′-pyrrolo[2,3-b]pyrazine]-2′-carbonyl)-3,3-dimethylpiperazin-1-yl)-2,4-dimethylnicotinic acid

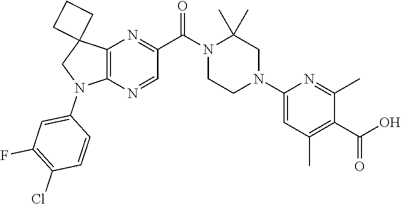 [SMILES: Cc1cc(N2CCN(C(=O)c3cnc4c(n3)C3(CCC3)CN4c3ccc(Cl)c(F)c3)C(C)(C)C2)nc(C)c1C(=O)O]

Example 237 was prepared according to general procedure (XI) starting from Example 236 (178 mg). The crude was purified by preparative HPLC (Column 1, H₂O+0.1% HCOOH/MeCN+0.1% HCOOH 70:30 to 0:100) and freeze dried with HCl (0.1N in water) to obtain Example 237 (15 mg, 6% over 2 steps) as a white solid.

¹H-NMR (DMSO-d₆, 300 MHz) δ: 1.52 (s, 6H, C(CH₃)₂); 1.96-2.20 (m, 2H, 2CHₐH_{b}); 2.29 (s, 3H, ArCH₃); 2.32-2.49 (m, 5H, 2CHₐH_{b}ArCH₃); 3.58-3.64 (m, 4H, CH₂N—CH₂); 3.83-3.99 (m, 4H, 2N—CH₂); 4.30 (s, 2H, N—CH₂); 6.52 (s, 1H, Ar); 7.57-7.72 (m, 2H, 2Ar); 8.02-8.13 (m, 1H, Ar); 8.22 (s, 1H, Ar); COOH was not observed.

M/Z (M[³⁵Cl]+H)⁺: 579.4.

### Example 238: methyl 2-(6-(4-(1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetate major isomer

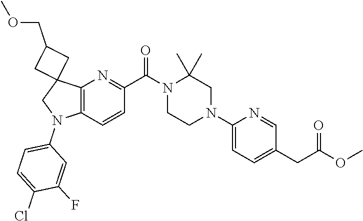

Example 238 was prepared according to general procedure (XIV) starting from Compound 287 (92 mg) and Compound 111 (1.5 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 238 (101 mg) as a yellow solid.

¹H-NMR (DMSO-d₆, 300 MHz) δ: 1.51 (s, 6H, C(CH₃)₂); 2.18-2.38 (m, 4H, 4CHₐH_{b}); 2.56-2.68 (m, 1H, CH); 3.26 (s, 3H, OCH₃); 3.48-3.57 (m, 4H, N—CH₂CH₂OMe); 3.58-3.64 (m, 5H, C(O)OCH₃N—CH₂); 3.80-3.93 (m, 4H, CH₂C(O)N—CH₂); 4.25 (s, 2H, N—CH₂); 6.60 (d, J 8.7 Hz, 1H, Ar); 7.19 (dd, J 9.0, 1.8 Hz, 1H, Ar); 7.30-7.46 (m, 3H, 3Ar); 7.50-7.60 (m, 2H, 2Ar); 7.95-7.97 (m, 1H, Ar).

M/Z (M[³⁵Cl]+H)⁺: 622.4

### Example 239: 2-(6-(4-(1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid major isomer

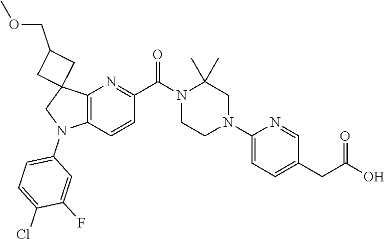 [SMILES: COCC1CC2(C1)CN(c1ccc(Cl)c(F)c1)c1ccc(C(=O)N3CCN(c4ccc(CC(=O)O)cn4)CC3(C)C)nc12]

Example 239 was prepared according to general procedure (VII) starting from Example 238 (101 mg). The crude was purified by preparative HPLC (Column A, H₂O+0.1% HCOOH/MeCN+0.1% HCOOH 65:35 to 35:65) and freeze dried with HCl (0.1N in water) to obtain Example 239 (70 mg, 49% over 2 steps) as a white solid.

¹H-NMR (DMSO-d₆, 300 MHz) δ: 1.57 (s, 6H, C(CH₃)₂); 2.16-2.37 (m, 4H, 4CHₐH_{b}); 2.56-2.68 (m, 1H, CH); 3.22 (s, 3H, OCH₃); 3.49 (d, J 7.5 Hz, 2H, CH₂OMe); 3.65 (s, 2H, CH₂C(O)); 3.71-3.79 (m, 2H, N—CH₂); 3.93-4.04 (m, 4H, 2N—CH₂); 4.25 (s, 2H, N—CH₂); 7.19 (dd, J 8.9, 2.6 Hz, 1H, Ar); 7.25-7.40 (m, 3H, 3Ar); 7.52-7.59 (m, 2H, 2Ar); 7.91-8.01 (m, 2H, 2Ar); COOH was not observed.

M/Z (M[³⁵Cl]+H)⁺: 608.4

### Example 240: methyl 2-(6-(4-(5-(4-chloro-3,5-difluorophenyl)-7,7-dimethyl-6,7-dihydro-5H-pyrrolo[2,3-b]pyrazine-2-carbonyl)-3,3-dimethylpiperazin-1-yl)pyridin-3-yl)acetate

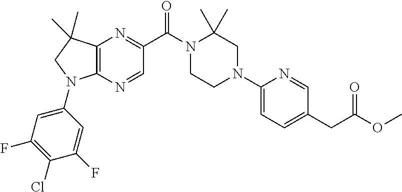 [SMILES: COC(=O)Cc1ccc(N2CCN(C(=O)c3cnc4c(n3)C(C)(C)CN4c3cc(F)c(Cl)c(F)c3)C(C)(C)C2)nc1]

Example 240 was prepared according to general procedure (XIV) starting from Compound 230 (150 mg) and Compound 111 (1.5 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 40:60) to obtain Example 240 (150 mg) as a yellow solid.

¹H-NMR (DMSO-d₆, 300 MHz) δ: 1.40 (s, 6H, C(CH₃)₂); 1.50 (s, 6H, C(CH₃)₂); 3.46-3.54 (m, 4H, N—CH₂CH₂—C(O)); 3.60 (s, 3H, OCH₃); 3.75-3.82 (m, 2H, N—CH₂); 3.85-3.93 (m, 2H, N—CH₂); 3.99 (s, 2H, N—CH₂); 4.00 (s, 2H, N—CH₂); 6.59 (d, J 8.4 Hz, 1H, Ar); 7.35-7.52 (m, 1H, 1Ar); 7.87-7.99 (m, 3H, 3Ar); 8.26 (s, 1H, Ar).

M/Z (M[³⁵Cl]+H)⁺: 585.4

### Example 241: 2-(6-(4-(5-(4-chloro-3,5-difluorophenyl)-7,7-dimethyl-6,7-dihydro-5H-pyrrolo[2,3-b]pyrazine-2-carbonyl)-3,3-dimethylpiperazin-1-yl)pyridin-3-yl)acetic acid

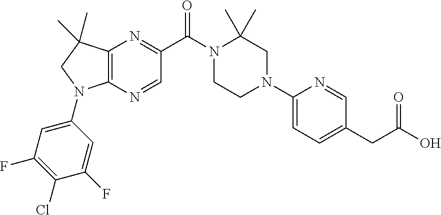 [SMILES: CC1(C)CN(c2cc(F)c(Cl)c(F)c2)c2ncc(C(=O)N3CCN(c4ccc(CC(=O)O)cn4)CC3(C)C)nc21]

Example 241 was prepared according to general procedure (VII) starting from Example 240 (150 mg). The crude was purified by preparative HPLC (Column B, H₂O+0.1% HCOOH/MeCN+0.1% HCOOH 70:30 to 30:70) and freeze dried with HCl (0.1N in water) to obtain Example 241 (90 mg, 37% over 2 steps) as a yellow solid.

¹H-NMR (DMSO-d₆, 300 MHz) δ: 1.40 (s, 6H, C(CH₃)₂); 1.55 (s, 6H, C(CH₃)₂); 3.63 (s, 2H, CH₂—C(O)); 3.66-3.74 (m, 2H, N—CH₂); 3.85-3.93 (m, 2H, N—CH₂); 3.97 (s, 2H, N—CH₂); 4.00 (s, 2H, N—CH₂); 7.28 (bs, 1H, Ar); 7.83-8.03 (m, 4H, 4Ar); 8.29 (s, 1H, Ar); COOH was not observed.

M/Z (M[³⁵Cl]+H)⁺: 571.4

### Compound 304: 5′-(4-chloro-3-fluorophenyl)-5′,6′-dihydrospiro[cyclopentane-1,7′-pyrrolo[2,3-b]pyrazine]-2′-carboxylic acid

In the first chamber of a two-chamber reactor (2*20 mL) was prepared a solution of Compound 263 (1.05 g) and triethyl amine (0.77 mL) in a dioxane/water mixture (1:1, 12 mL). In the second chamber a suspension of Mo(CO)₆ (445 mg) in dioxane (12 mL) was prepared. Both chambers were sparged with argon for 10 minutes then XantPhos Pd G4 (53 mg) was added in the first chamber, and DBU (0.74 mL) was added in the second chamber. The set up was crimped and heated at 85° C. for 16 hours. The mixture in the first chamber was filtered over a Celite pad, rinsed with EtOAc (40 mL) and water (10 mL), the filtrate was washed with water (2*30 mL). The aqueous washing were acidified with HCl (1N aq.) to pH=6, the solid was recovered by filtration, dried under high vacuum, taken up in DCM (10 mL) and combined with the organic layer. The combined organic layers were washed with brine (10 mL), dried over MgSO₄ and concentrated under reduced pressure to obtain Compound 304 (542 mg, 57%) as an orange solid.

M/Z (M[³⁵Cl]+H)⁺: 348.2

### Example 242: methyl 2-(6-(4-(5′-(4-chloro-3-fluorophenyl)-5′,6′-dihydrospiro[cyclopentane-1,7′-pyrrolo[2,3-b]pyrazine]-2′-carbonyl)-3,3-dimethylpiperazin-1-yl)pyridin-3-yl)acetate

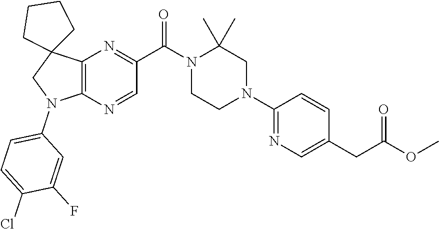 [SMILES: COC(=O)Cc1ccc(N2CCN(C(=O)c3cnc4c(n3)C3(CCCC3)CN4c3ccc(Cl)c(F)c3)C(C)(C)C2)nc1]

Example 242 was prepared according to general procedure (VIIIa) starting from Compound 304 (117 mg), Compound 111 (101 mg) and N,N-diisopropylethylamine (4.0 eq.) in DCM. The crude was purified by flash chromatography (Interchim® 50 μm, CyHex 100% to CyHex/EtOAc 50:50) to obtain Example 242 (103 mg, 52%) as a yellow solid.

M/Z (M[³⁵Cl]+H)⁺: 593.4.

### Example 243: 2-(6-(4-(5′-(4-chloro-3-fluorophenyl)-5′,6′-dihydrospiro[cyclopentane-1,7′-pyrrolo[2,3-b]pyrazine]-2′-carbonyl)-3,3-dimethylpiperazin-1-yl)pyridin-3-yl)acetic acid

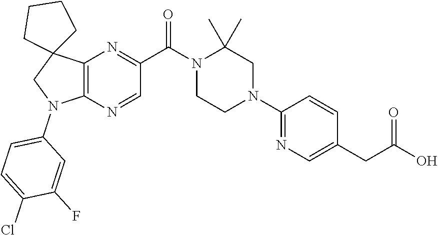 [SMILES: CC1(C)CN(c2ccc(CC(=O)O)cn2)CCN1C(=O)c1cnc2c(n1)C1(CCCC1)CN2c1ccc(Cl)c(F)c1]

To a solution of Example 242 (450 mg) in THE (2 mL) was added LiOH·H₂O (64 mg) in water (2 mL). The mixture was stirred at 20° C. for 45 minutes then diluted with EtOAc (10 mL) and treated with K₂CO₃ (sat. aq. 20 mL). The phases were separated and the aqueous layer was washed with Et₂O (10 mL) then acidified with HCl (1N, aq.) to pH=6.5 and extracted with DCM (3*15 mL). The combined organic layers were dried over MgSO₄ and concentrated under reduced pressure. The residue was dissolved in HCl (1N, aq. 10 mL), washed with MTBE (2*10 mL), then treated with K₂CO₃ (sat.aq.) to pH=6.5 and extracted with DCM (3*20 mL). The combined organic layers were dried over MgSO₄ and concentrated under reduced pressure to obtain Example 243 (272 mg, 62%) as a yellow solid.

¹H-NMR (DMSO-d₆, 300 MHz) δ: 1.50 (s, 6H, C(CH₃)₂); 1.81-2.00 (m, 8H, (CH₂)₄); 3.42 (s, 2H, CH₂—C(O)); 3.46-3.51 (m, 2H, N—CH₂); 3.78-3.85 (m, 4H, 2N—CH₂); 4.08 (s, 2H, N—CH₂); 6.57 (d, J 8.7 Hz, 1H, Ar); 7.44 (dd, J 8.7, 2.4 Hz, 1H, Ar); 7.62 (t, J 8.8 Hz, 1H, Ar); 7.76 (dd, J 8.8, 2.5 Hz, 1H, Ar); 7.94 (d, J 2.3 Hz, 1H, Ar); 8.08 (dd, J 8.8, 2.3 Hz, 1H, Ar); 8.21 (s, 1H, Ar); 12.39 (bs, 1H, COOH).

M/Z (M[³⁵Cl]+H)⁺: 579.4

### Example 244: methyl 2-(6-(4-(5′-(3,4-difluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)pyridin-3-yl)acetate

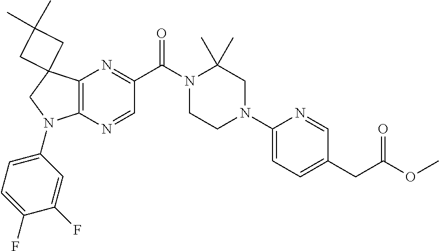 [SMILES: COC(=O)Cc1ccc(N2CCN(C(=O)c3cnc4c(n3)C3(CN4c4ccc(F)c(F)c4)CC(C)(C)C3)C(C)(C)C2)nc1]

Example 244 was prepared according to general procedure (XIV) starting from Compound 300 (733 mg) and Compound 111 (1.5 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 70:30 then DCM/MeOH 80:20) to obtain Example 244 (835 mg) as a red solid.

¹H-NMR (DMSO-d₆, 300 MHz) δ: 1.28 (s, 3H, C(CH₃)); 1.30 (s, 3H, C(CH₃)); 1.55 (s, 6H, C(CH₃)₂); 2.11-2.16 (m, 2H, 2CHₐH_{b}); 2.34-2.38 (m, 2H, 2CHₐH_{b}); 3.63-3.66 (m, 5H, N—CH₂OCH₃); 3.90-4.01 (m, 4H, 2N—CH₂CH₂C(O)); 4.40 (s, 2H, N—CH₂); 7.00 (bs, 1H, Ar); 7.46-7.56 (m, 1H, Ar); 7.70-7.75 (m, 2H, 2Ar); 7.95-7.97 (m, 1H, Ar); 8.08-8.16 (m, 1H, Ar); 8.20 (s, 1H, Ar); One N—CH₂ was not observed.

M/Z (M+H)⁺: 591.4

### Example 245: 2-(6-(4-(5′-(3,4-difluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)pyridin-3-yl)acetic acid

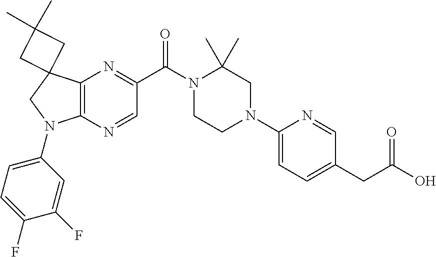 [SMILES: CC1(C)CC2(CN(c3ccc(F)c(F)c3)c3ncc(C(=O)N4CCN(c5ccc(CC(=O)O)cn5)CC4(C)C)nc32)C1]

To a solution of Example 244 (835 mg) in THE (4 mL) was added LiOH·H₂O (119 mg) in water (4 mL). The reaction mixture was stirred at 20° C. for 45 minutes then treated with HCl (1N, aq.) to pH=2 and diluted with MTBE (10 mL).

The solid was recovered by filtration and dried under high vacuum to obtain Example 245 (364 mg, 33%) as a beige solid.

¹H-NMR (DMSO-d₆, 300 MHz) δ: 1.29 (s, 6H, C(CH₃)₂); 1.51 (s, 6H, C(CH₃)₂); 2.11-2.16 (m, 2H, 2CHₐH_{b}); 2.34-2.38 (m, 2H, 2CHₐH_{b}); 3.43 (s, 2H, CH₂C(O)); 3.55-3.62 (m, 2H, N—CH₂); 3.79-3.92 (m, 4H, 2N—CH₂); 4.40 (s, 2H, N—CH₂); 6.56 (dd, J 8.7 Hz, 1H, Ar); 7.42-7.55 (m, 2H, 2Ar); 7.68-7.75 (m, 1H, Ar); 7.95 (m, 1H, Ar); 8.08-8.16 (m, 1H, Ar); 8.20 (s, 1H, Ar); 12.29 (bs, 1H, COOH).

M/Z (M+H)⁺: 577.5

### Compound 305: 4-chloro-N-((1-(3-chloropyrazin-2-yl)-3,3-dimethylcyclobutyl)methyl)-3-fluoroaniline

To a solution of Compound 297 (4.49 g), 4-chloro-3-fluoroaniline (3.49 g) and TFA (3.08 mL) in iPrOAc (200 mL) at 25° C. was added sodium triacetoxyhydroborate (5.09 g) portionwise over 10 minutes. The reaction mixture was stirred for 1 hour at 25° C., then diluted with EtOAc (100 mL), treated with NaOH (5N, aq.) to pH=9 and extracted with EtOAc (2*100 mL). The combined organic layers were dried over MgSO₄ and concentrated under reduced pressure to obtain Compound 305 (8.52 g) as brown oil.

M/Z (M[³¹Cl]₂+H)⁺: 354.2.

### Compound 306: 5′-(4-chloro-3-fluorophenyl)-3,3-dimethyl-5′,6′-dihydrospiro[cyclobutane-1,7′-pyrrolo[2,3-b]pyrazine]

Compound 306 was prepared according to general procedure (XXIII) from Compound 305 (8.52 g). The crude was purified by trituration in CyHex (30 mL) to obtain Compound 306 (3.44 g, 48%) as a beige solid.

M/Z (M[³⁵Cl]+H)⁺: 318.2

### Compound 307: 2′-bromo-5′-(4-chloro-3-fluorophenyl)-3,3-dimethyl-5′,6′-dihydrospiro[cyclobutane-1,7′-pyrrolo[2,3-b]pyrazine]

Compound 307 (3.67 g, 87%) was obtained as a beige solid following general procedure (XVI) in EtOAc, after purification by flash chromatography (Interchim® 50 μm, CyHex 100% to CyHex/EtOAc 90:10), starting from Compound 306 (3.40 g).

M/Z (M[³⁵Cl][⁸¹Br]+H)⁺: 398.1.

### Example 246: methyl 6-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-2,4-dimethylnicotinate

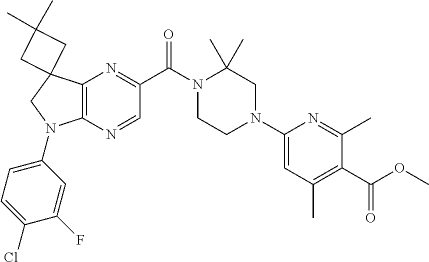 [SMILES: COC(=O)c1c(C)cc(N2CCN(C(=O)c3cnc4c(n3)C3(CN4c4ccc(Cl)c(F)c4)CC(C)(C)C3)C(C)(C)C2)nc1C]

Example 246 was prepared according to general procedure (XIV) starting from Compound 307 (1000 mg) and Compound 10 (1.5 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 70:30) to obtain Example 246 (944 mg) as a red solid.

¹H-NMR (DMSO-d₆, 300 MHz) δ: 1.30 (s, 6H, C(CH₃)₂); 1.51 (s, 6H, C(CH₃)₂); 2.12-2.16 (m, 2H, CHₐH_{b}); 2.23 (s, 3H, ArCH₃); 2.34-2.39 (m, 5H, 2CHₐH_{b}ArCH₃); 3.65-3.68 (m, 2H, N—CH₂); 3.78 (s, 3H, OCH₃); 3.85-3.90 (m, 4H, 2N—CH₂); 4.42 (s, 2H, N—CH₂); 6.35 (s, 1H, Ar); 7.58-7.64 (m, 1H, Ar); 7.78-7.82 (m, 1H, Ar); 8.08-8.13 (m, 1H, Ar); 8.23 (s, 1H, Ar).

M/Z (M[³⁵Cl]+H)⁺: 621.4.

### Example 247: 6-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-2,4-dimethylnicotinic acid

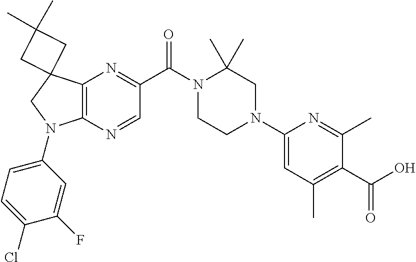 [SMILES: Cc1cc(N2CCN(C(=O)c3cnc4c(n3)C3(CN4c4ccc(Cl)c(F)c4)CC(C)(C)C3)C(C)(C)C2)nc(C)c1C(=O)O]

Example 247 was prepared according to general procedure (XI) starting from Example 246 (944 mg). The crude was purified by trituration in hot EtOH (5 mL) to obtain Example 247 (324 mg, 30% over 2 steps) as a white solid.

¹H-NMR (DMSO-d₆, 300 MHz) δ: 1.29 (s, 3H, C(CH₃)); 1.31 (s, 3H, C(CH₃)); 1.53 (s, 6H, C(CH₃)₂); 2.12-2.16 (m, 2H, CHₐH_{b}); 2.30 (s, 3H, ArCH₃); 2.35-2.39 (m, 2H, 2CHₐH_{b}); 2.45 (s, 3H, ArCH₃); 3.70-3.73 (m, 2H, N—CH₂); 3.89-3.93 (m, 4H, 2N—CH₂); 4.42 (s, 2H, N—CH₂); 6.58 (bs, 1H, Ar); 7.59-7.65 (m, 1H, Ar); 7.78-7.81 (m, 1H, Ar); 8.08-8.13 (m, 1H, Ar); 8.24 (s, 1H, Ar); COOH was not observed.

M/Z (M[³⁵Cl]+H)⁺: 607.4.

### Example 248: methyl 6-(4-(5-(3,4-dichlorophenyl)-7,7-dimethyl-6,7-dihydro-5H-pyrrolo[2,3-b]pyrazine-2-carbonyl)-3,3-dimethylpiperazin-1-yl)-2,4-dimethylnicotinate

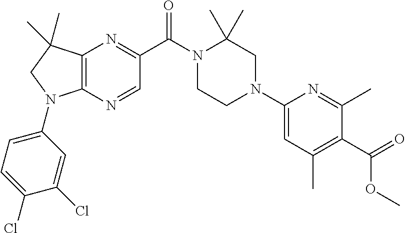 [SMILES: COC(=O)c1c(C)cc(N2CCN(C(=O)c3cnc4c(n3)C(C)(C)CN4c3ccc(Cl)c(Cl)c3)C(C)(C)C2)nc1C]

Example 248 was prepared according to general procedure (XIV) starting from Compound 279 (150 mg) and Compound 10 (1.5 eq), and using triethyl amine (4.0 eq), Mo(CO)₆ (1.5 eq), XantPhos Pd G4 (0.10 eq), and DBU (4.5 eq). The crude was purified by flash chromatography (Interchim® 20 μm, CyHex 100% to CyHex/EtOAc 30:70) to obtain Example 248 (174 mg) as a yellow solid.

¹H-NMR (DMSO-d₆, 300 MHz) δ: 1.39 (m, 6H, C(CH₃)₂); 1.51 (s, 6H, C(CH₃)₂); 2.23 (s, 3H, ArCH₃); 2.34 (m, 3H, ArCH₃); 3.54-3.57 (m, 2H, N—CH₂); 3.75-3.81 (m, 5H, N—CH₂OCH₃); 3.86-3.90 (m, 2H, N—CH₂); 4.00 (s, 2H, N—CH₂); 6.37 (s, 1H, Ar); 7.63-7.69 (m, 1H, Ar); 7.85-7.90 (m, 1H, Ar); 8.20-8.26 (m, 2H, 2Ar).

M/Z (M[³⁵Cl]₂+H)⁺: 623.4.

### Example 249: 6-(4-(5-(3,4-dichlorophenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid

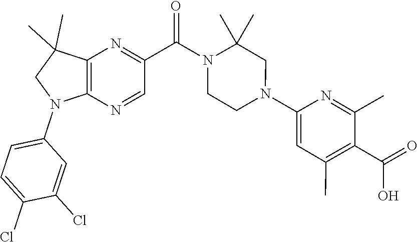 [SMILES: Cc1cc(N2CCN(C(=O)c3cnc4c(n3)C(C)(C)CN4c3ccc(Cl)c(Cl)c3)C(C)(C)C2)nc(C)c1C(=O)O]

Example 249 was prepared according to general procedure (XI) starting from Example 248 (157 mg). The crude was purified by preparative HPLC (Column B, H₂O+0.1% HCOOH/MeCN+0.1% HCOOH 65:35 to 00:100) and freeze dried with HCl (0.1N in water) to obtain Example 248 (88 mg, 35% over 2 steps) as a yellow solid.

¹H-NMR (DMSO-d₆, 300 MHz) δ: 1.39 (s, 6H, C(CH₃)₂); 1.51 (s, 6H, C(CH₃)₂); 2.31 (s, 3H, ArCH₃); 2.44 (s, 3H, ArCH₃); 3.60-3.64 (m, 2H, N—CH₂); 3.80-3.84 (m, 2H, N—CH₂); 3.91 (s, 2H, N—CH₂); 3.99 (s, 2H, N—CH₂); 6.62 (bs, 1H, Ar); 7.67 (d, J 9.0 Hz, 1H, Ar); 7.87 (dd, J 9.0; 2.4 Hz, 1H, Ar); 8.23 (s, 1H, Ar); 8.25 (d, J 2.4 Hz, 1H, Ar); COOH was not observed.

M/Z (M[³⁵Cl]₂+H)⁺: 583.3.

### Compound 308: 5-(3-chloro-4-methylphenyl)-7,7-dimethyl-6,7-dihydro-5H-pyrrolo[2,3-b]pyrazine-2-carboxylic acid

Compound 308 was prepared according to general procedure (XIV) starting from Compound 233 (150 mg) and Compound 111 (1.5 eq), and using triethyl amine (4.0 eq), Mo(CO)₆ (1.5 eq), XantPhos Pd G4 (0.10 eq), and DBU (4.5 eq). In this case the filtrate was extracted with K₂CO₃ (2M, aq., 2*5 mL) and the combined aqueous layers were freeze-dried to obtain crude Compound 308 (120 mg) as a white solid which was directly engaged in the following step.

M/Z (M[³⁵Cl]+H)⁺: 318.2

### Example 250: methyl 2-(6-(4-(5-(3-chloro-4-methylphenyl)-7,7-dimethyl-6,7-dihydro-5H-pyrrolo[2,3-b]pyrazine-2-carbonyl)-3,3-dimethylpiperazin-1-yl)pyridin-3-yl)acetate

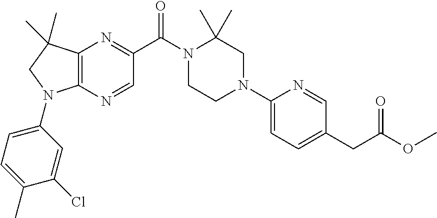 [SMILES: COC(=O)Cc1ccc(N2CCN(C(=O)c3cnc4c(n3)C(C)(C)CN4c3ccc(C)c(Cl)c3)C(C)(C)C2)nc1]

Example 250 was prepared according to general procedure (VIIIa) starting from Compound 308 (117 mg), Compound 111 (101 mg) and N,N-diisopropylethylamine (4.0 eq.) in DCM. The crude was purified by flash chromatography (Interchim® 50 μm, CyHex 100% to CyHex/EtOAc 50:50) to obtain Example 250 (103 mg, 52%) as a yellow solid.

M/Z (M[³⁵Cl]+H)⁺: 593.4.

### Example 251: 2-(6-(4-(5-(3-chloro-4-methylphenyl)-7,7-dimethyl-6,7-dihydro-5H-pyrrolo[2,3-b]pyrazine-2-carbonyl)-3,3-dimethylpiperazin-1-yl)pyridin-3-yl)acetic acid

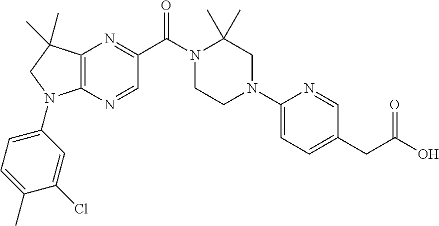 [SMILES: Cc1ccc(N2CC(C)(C)c3nc(C(=O)N4CCN(c5ccc(CC(=O)O)cn5)CC4(C)C)cnc32)cc1Cl]

Example 251 was prepared according to general procedure (VII) starting from Example 250 (101 mg). The crude was purified by preparative HPLC (Column A, H₂O+0.1% HCOOH/MeCN+0.1% HCOOH 65:35 to 35:65) and freeze dried with HCl (0.1N in water) to obtain Example 251 (70 mg, 49% over 2 steps) as a white solid.

¹H-NMR (DMSO-d₆, 300 MHz) δ: 1.29 (s, 6H, C(CH₃)₂); 1.51 (s, 6H, C(CH₃)₂); 2.11-2.16 (m, 2H, 2CHₐH_{b}); 2.34-2.38 (m, 2H, 2CHₐH_{b}); 3.43 (s, 2H, CH₂C(O)); 3.55-3.62 (m, 2H, N—CH₂); 3.79-3.92 (m, 4H, 2N—CH₂); 4.40 (s, 2H, N—CH₂); 6.56 (dd, J 8.7 Hz, 1H, Ar); 7.42-7.55 (m, 2H, 2Ar); 7.68-7.75 (m, 1H, Ar); 7.95 (m, 1H, Ar); 8.08-8.16 (m, 1H, Ar); 8.20 (s, 1H, Ar); 12.29 (bs, 1H, COOH).

M/Z (M+H)⁺: 577.5

### Compound 309: N-(2-bromopyridin-3-yl)-4-methylbenzenesulfonamide

To a solution of 2-bromopyridin-3-amine (10.00 g) in Pyridine (150 mL), was added 4-methylbenzenesulfonyl chloride (13.22 g) and the solution was heated at 40° C. for 6 hours. The mixture was diluted with water (300 mL) and extracted with EtOAc (2*300 mL). The combined organic layers were washed with brine (300 mL), dried over Na₂SO₄ and concentrated under reduced pressure. The crude was purified by flash chromatography (Petroleum ether/EtOAc 50:50) to obtain Compound 309 (10.0 g, 52%) as a yellow solid.

M/Z (M[⁷⁹Br]+H)⁺: 327.0.

### Compound 310: methyl 3-((N-(2-bromopyridin-3-yl)-4-methylphenyl)sulfonamido)-2,2-dimethylpropanoate

To a solution of Compound 309 (9.00 g) in THE (150 mL) was added PPh₃ (10.82 g) and methyl 3-hydroxy-2,2-dimethylpropanoate (4.36 g), then diisopropyl azodicarboxylate (11.12 g) was added portionwise at 0° C. and the mixture was stirred at 25° C. for 3 hours. The reaction mixture was diluted in water (300 mL) and extracted with EtOAc (2*300 mL). The combined organic layers were washed with brine (300 mL), dried over Na₂SO₄ and concentrated under reduced pressure. The crude was purified by flash chromatography (Petroleum ether/EtOAc 75:25) to obtain Compound 310 (9.00 g, 74%) as a yellow solid.

M/Z (M[⁷⁹Br]+H)⁺: 441.0.

### Compound 311: 3,3-dimethyl-1-tosyl-2,3-dihydro-1,5-naphthyridin-4(1H)-one

To a solution of Compound 310 (9.00 g) in THE (200 mL) at −78° C. under inert atmosphere was added slowly n-butyllithium (16.3 mL, 2.5 M in hexane). The mixture was stirred at −78° C. for 3 hours, then quenched in cooled water (200 mL, 0° C.) and extracted with EtOAc (2*200 mL). The combined organic layers were washed with brine (300 mL), dried over Na₂SO₄ and concentrated under reduced pressure. The crude was purified by flash chromatography (Petroleum ether/EtOAc 80:20) to obtain Compound 311 (4.50 g, 66%) as a white solid.

¹H NMR (DMSO-d₆, 400 MHz) δ: 1.17 (s, 6H, C(CH₃)₂); 2.40 (s, 3H, ArCH₃); 4.07 (s, 2H, N—CH₂); 7.48 (d, J 8.2 Hz, 2H, 2Ar); 7.53 (dd, J 8.6, 4.2 Hz, 1H, Ar); 7.94 (d, J 8.2 Hz, 2H, 2Ar); 8.02 (dd, J 8.6, 1.2 Hz, 1H); 8.44 (dd, J 4.2, 1.2 Hz, 1H).

M/Z (M+H)⁺: 331.1.

### Compound 312: 3,3-dimethyl-1-tosyl-1,2,3,4-tetrahydro-1,5-naphthyridin-4-ol

To a solution of Compound 311 (4.00 g) in THE (100 mL) was added portionwise NaBH₄ (0.92 g) at 25° C. and the mixture was stirred at 25° C. for 3 hours then quenched in cooled water (200 mL, 0° C.) and extracted with EtOAc (2*200 mL). The combined organic layers were washed with brine (200 mL), dried over Na₂SO₄ and concentrated under reduced pressure. The crude was purified by flash chromatography (Petroleum ether/EtOAc 75:25) to obtain Compound 312 (3.00 g, 74%) as a white solid.

M/Z (M+H)⁺: 333.1.

### Compound 313: 4-methoxy-3,3-dimethyl-1-tosyl-1,2,3,4-tetrahydro-1,5-naphthyridine

To a solution of Compound 312 (3.00 g) in THE (50 mL) was added NaH (0.72 g, 60% in mineral oil) at 0° C. portionwise under inert atmosphere. The mixture was stirred at 0° C. for 30 minutes then iodomethane (2.56 g) was added and the mixture was stirred for 3 hours at 25° C. then quenched in cooled water (100 mL, 0° C.) and extracted with EtOAc (2*100 mL). The combined organic layers were washed with brine (100 mL), dried over Na₂SO₄ and concentrated under reduced pressure. The crude was purified by flash chromatography (Petroleum ether/EtOAc 84:16) to obtain Compound 313 (2.70 g, 86%) as a white solid.

M/Z (M+H)⁺: 346.1.

### Compound 314: 4-methoxy-3,3-dimethyl-1,2,3,4-tetrahydro-1,5-naphthyridine

To a solution of Compound 313 (2.50 g) in MeOH (50 mL) was added Magnesium (1.75 g) and the mixture was refluxed for 6 hours, then filtered and the filtrate was concentrated under reduced pressure. The residue was diluted with water (50 mL) and extracted with EtOAc (2*50 mL). The combined organic layers were washed with brine (100 mL), dried over Na₂SO₄ and concentrated under reduced pressure. The crude was purified by flash chromatography (Petroleum ether/EtOAc 50:50) to obtain Compound 314 (1.30 g, 93%) as a white solid.

M/Z (M+H)⁺: 193.1.

### Compound 315: 1-(4-chloro-3-fluorophenyl)-4-methoxy-3,3-dimethyl-1,2,3,4-tetrahydro-1,5-naphthyridine

To a solution of Compound 314 (1.20 g) in Toluene (20 mL) was added 1-chloro-2-fluoro-4-iodobenzene (8.00 g), Pd(PPh₃)₄ (1.44 g) and Sodium tert-butoxide (1.80 g), the mixture was sparged with Argon for 2 minutes then refluxed for 6 hours. The mixture was diluted with water (50 mL) and extracted with EtOAc (2*50 mL). The combined organic layers were washed with brine (50 mL), dried over Na₂SO₄ and concentrated under reduced pressure. The crude was purified by flash chromatography (Petroleum ether/EtOAc 75:25) to obtain Compound 315 (0.60 g, 30%) as a white solid.

¹H NMR (CDCl₃, 400 MHz) δ: 0.89-0.93 (m, 3H, C(CH₃)ₐ(CH₃)_{b}); 1.12-1.16 (m, 3H, C(CH₃)ₐ(CH₃)_{b}); 2.98-3.02 (m, 1H, CHₐH_{b}); 3.51 (s, 3H, OCH₃); 3.65-3.68 (m, 1H, CHₐH_{b}); 3.78 (s, 1H, CH—OCH₃); 6.95-7.07 (m, 4H, 4Ar); 7.35-7.42 (m, 1H, Ar); 7.99-8.03 (m, 1H, Ar).

M/Z (M[³⁵Cl]+H)⁺: 321.1.

### Compound 316: 6-bromo-1-(4-chloro-3-fluorophenyl)-4-methoxy-3,3-dimethyl-1,2,3,4-tetrahydro-1,5-naphthyridine

To a solution of Compound 315 (600 mg) in THE (10 mL) was added N-bromo-succinimide (128 mg) and Acetic acid (112 mg), the mixture was stirred at 25° C. for 3 hours then diluted with water (20 mL) and extracted with EtOAc (2*20 mL). The combined organic layers were washed with brine (20 mL), dried over Na₂SO₄ and concentrated under reduced pressure. The crude was purified by flash chromatography (Petroleum ether/EtOAc 75:25) to obtain Compound 316 (550 mg, 73%) as a white solid.

M/Z (M[⁷⁹Br][³⁵Cl]+H)⁺: 399.0.

### Example 252: methyl 2-(6-(4-(5-(4-chloro-3-fluorophenyl)-8-methoxy-7,7-dimethyl-5,6,7,8-tetrahydro-1,5-naphthyridine-2-carbonyl)-3,3-dimethylpiperazin-1-yl)pyridin-3-yl)acetate

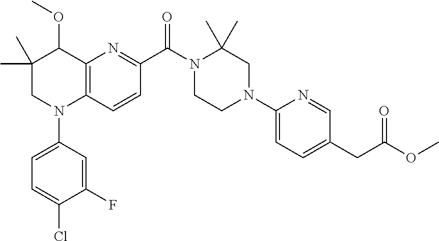

To a solution of Compound 316 (550 mg) in dioxane (10 mL) was added Compound 111 (435 mg), XantPhos Pd G4 (1100 mg) and NEt₃ (418 mg). The reaction was heated at 110° C. for 6 hours under CO atmosphere, then diluted with water (20 mL) and extracted with EtOAc (2*20 mL). The combined organic layers were washed with brine (50 mL), dried over Na₂SO₄ and concentrated under reduced pressure. The crude was purified by flash chromatography (Petroleum ether/EtOAc 50:50) to obtain Example 252 (430 mg, 51%) as a white solid.

¹H NMR (DMSO-d₆, 400 MHz) δ: 0.83-0.91 (m, 3H, C(CH₃)ₐ(CH₃)_{b}); 1.07-1.13 (m, 3H, C(CH₃)ₐ(CH₃)_{b}); 1.47-1.52 (m, 6H, C(CH₃)₂); 3.13-3.19 (m, 1H, CHₐH_{b}); 3.40-3.46 (m, 5H, OCH₃N—CH₂); 3.46-3.58 (m, 2H, N—CH₂); 3.59-3.64 (m, 4H, CH₂—C(O)N—CH₂); 3.74-3.81 (m, 1H, CHₐH_{b}); 3.83 (s, 3H, C(O)OCH₃); 3.93-4.03 (m, 1H, CH—OCH₃); 6.58-6.62 (m, 1H, Ar); 7.11-7.15 (m, 1H, Ar); 7.18-7.24 (m, 1H, Ar); 7.34-7.39 (m, 1H, Ar); 7.42-7.50 (m, 2H, 2Ar); 7.60-7.66 (m, 1H, Ar); 7.96 (d, J 2.4 Hz, 1H, Ar).

M/Z (M[³⁵Cl]+H)⁺: 610.2.

### Example 253: 2-(6-(4-(5-(4-chloro-3-fluorophenyl)-8-methoxy-7,7-dimethyl-5,6,7,8-tetrahydro-1,5-naphthyridine-2-carbonyl)-3,3-dimethylpiperazin-1-yl)pyridin-3-yl)acetic acid

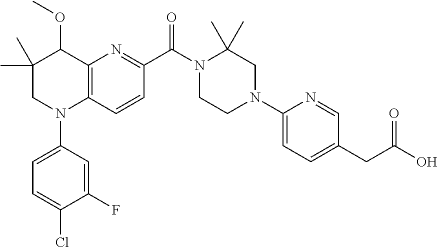 [SMILES: COC1c2nc(C(=O)N3CCN(c4ccc(CC(=O)O)cn4)CC3(C)C)ccc2N(c2ccc(Cl)c(F)c2)CC1(C)C]

To a solution of Example 252 (400 mg) in THE (10 mL) was added a solution of LiOH (314 mg) in water (4 mL) and the mixture was stirred at 25° C. for 3 hours, then the mixture was acidified to pH=6 with HCl (0.5 M aq.) and extracted with EtOAc (2*10 mL). The combined organic layers were dried over Na₂SO₄ and concentrated under reduced pressure. The crude was purified by preparative HPLC (H₂O/MeCN+0.05% HCOOH) to obtain Example 253 (145 mg, 37%) as a white solid.

¹H NMR (DMSO-d₆, 400 MHz) δ: 0.83-0.91 (m, 3H, C(CH₃)ₐ(CH₃)_{b}); 1.07-1.13 (m, 3H, C(CH₃)ₐ(CH₃)_{b}); 1.47-1.52 (m, 6H, C(CH₃)₂); 3.13-3.19 (m, 1H, CHₐH_{b}); 3.40-3.46 (m, 5H, OCH₃N—CH₂); 3.46-3.58 (m, 2H, N—CH₂); 3.58-3.64 (m, 1H, CHₐH_{b}); 3.75-3.78 (m, 1H, CH—OCH₃); 3.79-4.03 (m, 4H, CH₂—C(O)N—CH₂); 6.58-6.62 (m, 1H, Ar); 7.11-7.15 (m, 1H, Ar); 7.18-7.24 (m, 1H, Ar); 7.34-7.39 (m, 1H, Ar); 7.42-7.50 (m, 2H, 2Ar); 7.60-7.66 (m, 1H, Ar); 7.95 (d, J 2.4 Hz, 1H, Ar); 12.27 (bs, 1H, COOH).

M/Z (M[³⁵Cl]+H)⁺: 596.2.

### Compound 317: 2-(5-chloro-3-fluoropyridin-2-yl)-2-methylpropanenitrile

To a solution of 5-chloro-2,3-difluoropyridine (25.00 g) and isobutyronitrile (23.17 g) in Toluene (500 mL) was added slowly at 0° C. NaHMDS (1M in THF, 167 mL) under inert atmosphere. The reaction was stirred for 16 hours at 25° C. then quenched with cooled water (300 mL, 0° C.) and extracted with EtOAc (2*300 mL). The combined organic layers were washed with brine (300 mL), dried over Na₂SO₄ and concentrated under reduced pressure. The crude was purified by flash chromatography (Petroleum ether/EtOAc 90:10) to obtain Compound 317 (27.20 g, 82%) as a white solid.

M/Z (M[³⁵Cl]+H)⁺: 199.2.

### Compound 318: 2-(5-chloro-3-fluoropyridin-2-yl)-2-methylpropan-1-amine

To Compound 317 (26.20 g) was added BH₃-THF (1M in THF, 265 mL) at 0° C. under inert atmosphere. The mixture was heated at 60° C. for 4 hours then quenched by slow addition in cooled water (300 mL, 0° C.). The mixture was freeze dried to obtain crude Compound 318 (n.d.) directly used in the following step.

### Compound 319: 6-chloro-3,3-dimethyl-2,3-dihydro-1H-pyrrolo[3,2-b]pyridine

To a solution of crude Compound 318 (7.00 g) in NMP (70 mL) was added K₂CO₃ (24.00 g), the mixture was heated at 150° C. for 3 hours then diluted with water (300 mL) and extracted with EtOAc (2*300 mL). The combined organic layers were washed with brine (300 mL), dried over Na₂SO₄ and concentrated under reduced pressure. The crude was purified by flash chromatography (Petroleum ether/EtOAc 80:20) to obtain Compound 319 (0.83 g, 13%) as a white solid.

¹H NMR (DMSO-d₆, 400 MHz) δ: 1.21 (s, 6H, C(CH₃)₂); 3.31 (d, J 1.8 Hz, 2H, N—CH₂); 6.07 (d, J 2.0 Hz, 1H, Ar); 6.74 (d, J 1.8 Hz, 1H, NH); 7.60 (d, J 2.0 Hz, 1H, Ar).

M/Z (M[³⁵Cl]+H)⁺: 183.2.

### Compound 320: tert-butyl 6-chloro-3,3-dimethyl-2,3-dihydro-1H-pyrrolo[3,2-b]pyridine-1-carboxylate

To a solution of Compound 319 (3.30 g) in THE (30 mL) was added NEt₃ (7.40 g) and Boc₂O (15.80 g). The mixture was heated at 60° C. for 16 hours then diluted with water (100 mL) and extracted with EtOAc (2*100 mL). The combined organic layers were washed with brine (100 mL), dried over Na₂SO₄ and concentrated under reduced pressure. The crude was purified by flash chromatography (Petroleum ether/EtOAc 90:10) to obtain Compound 320 (1.50 g, 29%) as a white solid.

M/Z (M[³⁵Cl]+H)⁺: 283.2.

### Compound 321: tert-butyl 6-methoxy-3,3-dimethyl-2,3-dihydro-1H-pyrrolo[3,2-b]pyridine-1-carboxylate

To a solution of Compound 320 (1.10 g) in Dioxane (10 mL) was added Sodium tert-butoxide (0.53 g), MeOH (0.63 g), and tBuBrettPhos Pd G3 (0.33 g). The mixture was sparged with Argon for 1 minute then irradiated at 50° C. in a microwave reactor. The mixture was filtered over celite, the solids were rinsed with EtOAc (30 mL) and the combined filtrates were concentrated under reduced pressure. The crude was purified by flash chromatography (Petroleum ether/EtOAc 80:20) to obtain Compound 321 (0.65 g, 60%) as a white solid.

M/Z (M+H)⁺: 279.2.

### Compound 322: tert-butyl 5-bromo-6-methoxy-3,3-dimethyl-2,3-dihydro-1H-pyrrolo[3,2-b]pyridine-1-carboxylate

To a solution of Compound 321 (650 mg) in DMF (5 mL) was added N-bromosuccinimide (525 mg) and the reaction was stirred at 25° C. for 2 hours then diluted with water (30 mL) and extracted with EtOAc (2*30 mL). The combined organic layers were washed with brine (30 mL), dried over Na₂SO₄ and concentrated under reduced pressure. The crude was purified by flash chromatography (Petroleum ether/EtOAc 80:20) to obtain Compound 322 (800 mg, 96%) as a yellow solid.

M/Z (M[⁷⁹Br]+H)⁺: 357.2.

### Compound 323: 1-(tert-butyl) 5-methyl 6-methoxy-3,3-dimethyl-2,3-dihydro-1H-pyrrolo[3,2-b]pyridine-1,5-dicarboxylate

To a solution of Compound 322 (500 mg) in MeOH (5 mL) was added Xphos (200 mg), Pd(OAc)₂ (200 mg) and NEt₃ (568 mg). The reaction was heated at 70° C. for 48 hours under CO atmosphere, then filtered and concentrated under reduced pressure. The crude was purified by flash chromatography (Petroleum ether 100% to Petroleum ether/EtOAc 50:50) to obtain Compound 323 (300 mg, 60%) as a white solid.

M/Z (M+H)⁺: 337.2.

### Compound 334: methyl 6-methoxy-3,3-dimethyl-2,3-dihydro-1H-pyrrolo[3,2-b]pyridine-5-carboxylate

To a solution of Compound 323 (400 mg) in DCM (4 mL) was added TFA (2.0 mL). The mixture was stirred at 25° C. for 2 hours then concentrated under reduced pressure. The residue was quenched in NaHCO₃ (sat. aq. 10 mL) and extracted with EtOAc (2*20 mL). The combined organic layers were washed with brine (30 mL), dried over Na₂SO₄ and concentrated under reduced pressure to obtain crude Compound 334 (290 mg) as a yellow oil.

M/Z (M+H)⁺: 237.2.

### Compound 335: 1-(4-chloro-3-fluorophenyl)-6-methoxy-3,3-dimethyl-2,3-dihydro-1H-pyrrolo[3,2-b]pyridine-5-carboxylic acid

To a solution of Compound 334 (290 mg) and 1-chloro-2-fluoro-4-iodobenzene (1.57 g) in Toluene (10 mL) was added Sodium tert-butoxide (354 mg), XPhos (59 mg) and Pd(dppf)Cl₂·DCM (90 mg). The mixture was sparged with argon for 3 minutes then heated at 100° C. for 16 hours. The mixture was filtered and the filtrate concentrated under reduced pressure. The residue was purified by flash chromatography (C18 as stationary phase, MeOH/water 40:60) to obtain Compound 335 (270 mg, 63% over 2 steps) as a brown solid.

¹H NMR (DMSO-d₆, 400 MHz) δ: 1.26 (s, 6H, C(CH₃)₂); 3.72 (s, 3H, OCH₃); 3.74 (s, 2H, N—CH₂); 7.02 (s, 1H, Ar); 7.10-7.15 (m, 1H, Ar); 7.18-7.25 (m, 1H, Ar); 7.46-7.50 (m, 1H, Ar); COOH was not observed.

M/Z (M+H)⁺: 351.2.

### Example 254: methyl 2-(6-(4-(1-(4-chloro-3-fluorophenyl)-6-methoxy-3,3-dimethyl-2,3-dihydro-1H-pyrrolo[3,2-b]pyridine-5-carbonyl)-3,3-dimethylpiperazin-1-yl)pyridin-3-yl)acetate

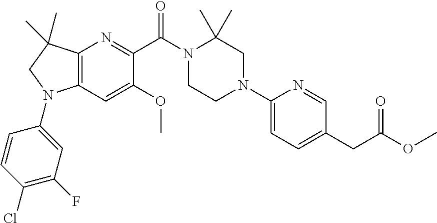 [SMILES: COC(=O)Cc1ccc(N2CCN(C(=O)c3nc4c(cc3OC)N(c3ccc(Cl)c(F)c3)CC4(C)C)C(C)(C)C2)nc1]

To a solution of Compound 335 (270 mg) and Compound 111 (243 mg) in DMF (10 mL) was added HATU (439 mg) and NEt(iPr)₂ (300 mg), the mixture was stirred at 25° C. for 2 hours then diluted with water (50 mL) and extracted with EtOAc (2*30 mL). The combined organic layers were washed with brine (50 mL), dried over Na₂SO₄ and concentrated under reduced pressure. The crude was purified by flash chromatography (DCM/MeOH 90:10) to obtain Example 254 (270 mg, 59%) as a yellow solid.

M/Z (M[³⁵Cl]+H)⁺: 596.2.

### Example 255: 2-(6-(4-(1-(4-chloro-3-fluorophenyl)-6-methoxy-3,3-dimethyl-2,3-dihydro-1H-pyrrolo[3,2-b]pyridine-5-carbonyl)-3,3-dimethylpiperazin-1-yl)pyridin-3-yl)acetic acid

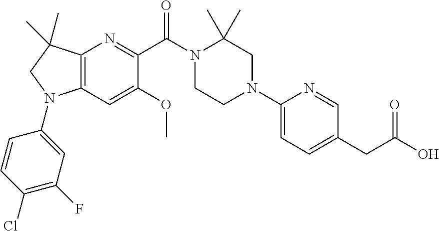 [SMILES: COc1cc2c(nc1C(=O)N1CCN(c3ccc(CC(=O)O)cn3)CC1(C)C)C(C)(C)CN2c1ccc(Cl)c(F)c1]

To a solution of Example 254 (60 mg) in THE (2 mL) was added LiOH (2M in water, 2 mL), the mixture was stirred at 70° C. for 2 hours then filtered and concentrated. The residue was purified by preparative HPLC (H₂O/MeCN+0.05% HCOOH) to obtain Example 255 (25 mg, 43%) as a white solid.

¹H NMR (DMSO-d₆, 400 MHz) δ: 1.31 (s, 6H, C(CH₃)₂); 1.50 (s, 6H, C(CH₃)₂); 3.29-3.31 (m, 2H, N—CH₂); 3.35-3.40 (m, 2H, N—CH₂); 3.41 (s, 2H, N—CH₂); 3.50 (s, 2H, N—CH₂); 3.83 (s, 3H, OCH₃); 3.84 (s, 2H, CH₂C(O)); 6.53-6.59 (m, 1H, Ar); 7.15 (s, 1H, Ar); 7.19-7.26 (m, 1H, Ar); 7.34-7.39 (m, 1H, Ar); 7.42-7.49 (m, 1H, Ar); 7.52-7.58 (m, 1H, Ar); 7.94 (d, J 2.2 Hz, 1H, Ar); 12.30 (bs, 1H, COOH).

M/Z (M[³⁵Cl]+H)⁺: 582.2

### Compound 336: 4-(3-bromoprop-1-en-2-yl)-1-chloro-2-fluorobenzene

To a solution of 1-chloro-2-fluoro-4-(prop-1-en-2-yl)benzene (7.50 g) in THE (150 mL) was added N-bromosuccinimide (11.74 g) and Acetic acid (2.64 g), the mixture was stirred at 25° C. for 3 hours then diluted with water (200 mL) and extracted with EtOAc (2*200 mL). The combined organic layers were washed with brine (200 mL), dried over Na₂SO₄ and concentrated under reduced pressure. The crude was purified by flash chromatography (Petroleum Ether/EtOAc 91:9) to obtain Compound 336 (6.00 g, 54%) as a yellow solid.

M/Z (M[⁷⁹Br][³⁵Cl]+H)⁺: 248.9.

### Compound 337: 6-chloro-N-(2-(4-chloro-3-fluorophenyl)allyl)-3-iodopyridin-2-amine

To a solution of Compound 336 (6.00 g) and 6-chloro-3-iodopyridin-2-amine (7.34 g) in THE (100 mL) was added Potassium tert-butoxide (5.40 g), the mixture wat stirred at 25° C. for 3 hours under inert atmosphere then quenched with water (200 mL) and extracted with EtOAc (2*200 mL). The combined organic layers were washed with brine (300 mL), dried over Na₂SO₄ and concentrated under reduced pressure. The crude was purified by flash chromatography (Petroleum Ether/EtOAc 67:33) to obtain Compound 337 (3.50 g, 34%) as a white solid.

M/Z (M[³⁵Cl]₂+H)⁺: 422.9

### Compound 338: 6-chloro-3-(4-chloro-3-fluorophenyl)-3-methyl-2,3-dihydro-1H-pyrrolo[2,3-b]pyridine

To a solution of Compound 337 (1.50 g), tetrabutylammonium bromide (1.37 g), NEt₃ (1.08 g), and Potassium formate (597 mg) in DMF (30 mL) was added Pd(OAc)₂ (398 mg). The mixture was sparged with Argon for 2 minutes, heated at 110° C. for 3 hours under inert atmosphere, then diluted with water (50 mL) and extracted with EtOAc (2*50 mL).

The combined organic layers were washed with brine (50 mL), dried over Na₂SO₄ and concentrated under reduced pressure. The crude was purified by flash chromatography (Petroleum Ether/EtOAc 50:50) to obtain Compound 338 (200 mg, 19%) as a white solid.

¹H NMR (CDCl₃, 400 MHz) δ: 1.68 (s, 3H, C(CH₃)); 3.65-3.69 (m, 1H, N—CHₐH_{b}); 3.73-3.77 (m, 1H, N—CHₐH_{b}); 5.34 (bs, 1H, NH); 6.55-6.59 (m, 1H, Ar); 7.00-7.05 (m, 2H, 2Ar); 7.07-7.13 (m, 1H, Ar); 7.30-7.35 (m, 1H, Ar).

M/Z (M[³⁵Cl]₂+H)⁺: 297.0

### Compound 339: 6-chloro-3-(4-chloro-3-fluorophenyl)-1-isobutyl-3-methyl-2,3-dihydro-1H-pyrrolo[2,3-b]pyridine

To a solution of Compound 338 (200 mg) in THE (5 mL) was added 1-bromo-2-methylpropane (185 mg) and Potassium tert-butoxide (151 mg). The mixture was heated at 60° C. for 3 hours under inert atmosphere then quenched with water (10 mL) and extracted with EtOAc (2*10 mL). The combined organic layers were concentrated under reduced pressure. The crude was purified by flash chromatography (Petroleum Ether/EtOAc 75:25) to obtain Compound 339 (90 mg, 37%) as a white solid.

¹H NMR (CDCl₃, 400 MHz) δ: 0.90-0.96 (m, 6H, C(CH₃)₂); 1.65 (s, 3H, C(CH₃)); 1.86-2.00 (m, 1H, CH); 3.16-3.26 (m, 2H, CH₂—CH); 3.48-3.54 (m, 1H, N—CHₐH_{b}); 3.55-3.61 (m, 1H, N—CHₐH_{b}); 6.44-6.48 (m, 1H, Ar); 6.88-6.93 (m, 1H, Ar); 6.98-7.02 (m, 1H, Ar); 7.05-7.11 (m, 1H, Ar); 7.30-7.35 (m, 1H, Ar).

M/Z (M[³⁵Cl]₂+H)⁺: 353.0

### Example 256: methyl 2-(6-(4-(3-(4-chloro-3-fluorophenyl)-1-isobutyl-3-methyl-2,3-dihydro-1H-pyrrolo[2,3-b]pyridine-6-carbonyl)-3,3-dimethylpiperazin-1-yl)pyridin-3-yl)acetate

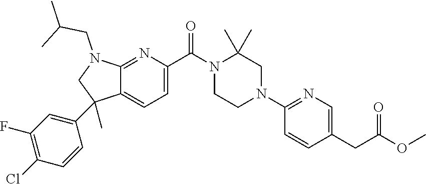 [SMILES: COC(=O)Cc1ccc(N2CCN(C(=O)c3ccc4c(n3)N(CC(C)C)CC4(C)c3ccc(Cl)c(F)c3)C(C)(C)C2)nc1]

To a solution of Compound 339 (80 mg) in Dioxane (3 mL) was added Compound 111 (72 mg), NEt₃ (69 mg), and XantPhos Pd G4 (367 mg). The mixture was heated at 110° C. for 16 hours under CO atmosphere then diluted with water (50 mL) and extracted with EtOAc (2*50 mL). The combined organic layers were dried over Na₂SO₄ and concentrated under reduced pressure. The crude was purified by flash chromatography (Petroleum Ether/EtOAc 50:50) to obtain Example 256 (70 mg, 50%) as a white solid.

M/Z (M[³⁵Cl]+H)⁺: 608.3

### Example 257: 2-(6-(4-(3-(4-chloro-3-fluorophenyl)-1-isobutyl-3-methyl-2,3-dihydro-1H-pyrrolo[2,3-b]pyridine-6-carbonyl)-3,3-dimethylpiperazin-1-yl)pyridin-3-yl)acetic acid

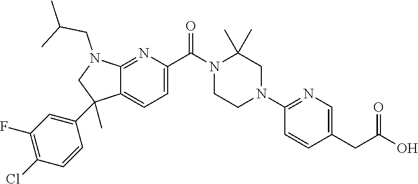 [SMILES: CC(C)CN1CC(C)(c2ccc(Cl)c(F)c2)c2ccc(C(=O)N3CCN(c4ccc(CC(=O)O)cn4)CC3(C)C)nc21]

To a solution of Example 256 (70 mg) in THE (3 mL) was added LiOH (28 mg) in water (1 mL). The mixture was stirred at 25° C. for 3 hours then acidified to pH=6 with HCl (aq. 0.5 M) and extracted with EtOAc (2*10 mL). The combined organic layers were dried over Na₂SO₄ and concentrated under reduced pressure. The residue was purified by preparative HPLC (H₂O/MeCN+0.05% HCOOH) to obtain Example 257 (26 mg, 38%) as a white solid.

¹H NMR (DMSO-d₆, 400 MHz) δ: 0.87-0.93 (m, 6H, CH(CH₃)₂); 1.47 (s, 6H, C(CH₃)₂); 1.66 (s, 3H, C(CH₃)); 1.93-2.02 (m, 1H, CH); 3.08-3.14 (m, 1H, CHₐH_{b}—CH); 3.17-3.24 (m, 1H, CHₐH_{b}—CH); 3.41 (s, 2H, N—CH₂); 3.46-3.48 (m, 2H, N—CH₂); 3.55 (d, J 9.6 Hz, 1H, N—CHₐH_{b}); 3.68-3.77 (m, 3H, N—CHₐH_{b} N—CH₂); 3.84 (s, 2H, CH₂C(O)); 6.53-6.57 (m, 1H, Ar); 6.63-6.67 (m, 1H, Ar); 7.15-7.19 (m, 1H, Ar); 7.29-7.33 (m, 1H, Ar); 7.38-7.44 (m, 2H, 2Ar); 7.52-7.58 (m, 1H, Ar); 7.94 (s, 1H, Ar); 12.25 (bs, 1H, COOH).

M/Z (M[³⁵Cl]+H)⁺: 594.3

### Example 258: 2-(6-(4-(1-(4-chloro-3-fluorophenyl)-6-hydroxy-3,3-dimethyl-2,3-dihydro-1H-pyrrolo[3,2-b]pyridine-5-carbonyl)-3,3-dimethylpiperazin-1-yl)pyridin-3-yl)acetic acid

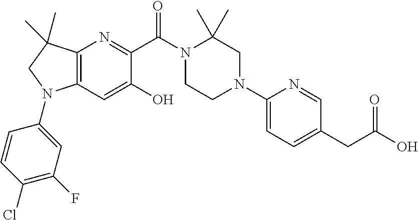 [SMILES: CC1(C)CN(c2ccc(Cl)c(F)c2)c2cc(O)c(C(=O)N3CCN(c4ccc(CC(=O)O)cn4)CC3(C)C)nc21]

A solution of Example 255 (120 mg) in DCM (5 mL) was sparged with Argon for 3 minutes then BBr₃ (1M in DCM, 3.1 mL) was added at 0° C. and the reaction mixture was stirred at 25° C. for 3 days then quenched with water (10 mL) and extracted with EtOAc (2*20 mL). The combined organic layers were washed with brine (20 mL), dried over Na₂SO₄ and concentrated under reduced pressure. The residue was purified by preparative HPLC (H₂O/MeCN+0.05% HCOOH) to obtain Example 258 (6 mg, 5%) as a yellow solid.

¹H NMR (DMSO-d₆, 400 MHz) δ: 1.32 (s, 6H, C(CH₃)₂); 1.51 (s, 6H, C(CH₃)₂); 3.46 (bs, 2H, N—CH₂); 3.56 (bs, 2H, N—CH₂); 3.85 (bs, 2H, N—CH₂); 3.86 (s, 2H, CH₂C(O)); 4.06 (bs, 2H, N—CH₂); 6.63 (bs, 1H, Ar); 6.97 (bs, 1H, Ar); 7.16-7.20 (m, 1H, Ar); 7.28-7.36 (m, 1H, Ar); 7.44 (bs, 1H, Ar); 7.55-7.61 (m, 1H, Ar); 7.95 (d, J 2.0 Hz, 1H, Ar); 11.33 (bs, 1H, OH); 12.31 (bs, 1H, COOH).

M/Z (M[³⁵Cl]+H)⁺: 568.2

### Compound 340: tert-butyl 4-(8-methoxy-8-oxooctanoyl)-2,2-dimethylpiperazine-1-carboxylate

To a solution of 8-methoxy-8-oxooctanoic acid (439 mg), HATU (1240 mg) and NEt(iPr)₂ (1.2 mL) in DCM (7.8 mL) was added tert-butyl 2,2-dimethylpiperazine-1-carboxylate (500 mg). The mixture was stirred at 25° C. for 18 hours then washed with HCl (1N aq. 20 mL), with NaHCO₃ (sat. aq. 20 mL), and with brine (20 mL), the combined organic layer were dried over MgSO₄ and concentrated under reduced pressure. The residue was purified by flash chromatography (Interchim® 50 μm, CyHex 100% to CyHex/EtOAc 60:40) to obtain Compound 340 (649 mg, 72%) as a clear oil.

M/Z (M+H)⁺: 385.4.

### Compound 341: methyl 8-(3,3-dimethylpiperazin-1-yl)-8-oxooctanoate hydrochloride

To a solution of Compound 340 (645 mg) in DCM (8.3 mL) was added HCl (2M in Et₂O, 8.3 mL) and the mixture was stirred at 25° C. for 18 hours then concentrated under reduced pressure to obtain Compound 341 (522 mg, 97%) as a clear oil.

M/Z (M+H)⁺: 285.4.

### Example 259: methyl 8-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-8-oxooctanoate

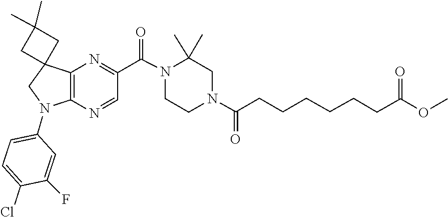 [SMILES: COC(=O)CCCCCCC(=O)N1CCN(C(=O)c2cnc3c(n2)C2(CN3c3ccc(Cl)c(F)c3)CC(C)(C)C2)C(C)(C)C1]

Example 259 was prepared according to general procedure (XIV) starting from Compound 307 (150 mg) and Compound 341 (1.5 eq), and using triethyl amine (3.0 eq), Mo(CO)₆ (1.5 eq), XantPhos Pd G4 (0.10 eq), and DBU (4.5 eq). The crude was purified by flash chromatography (Interchim® 20 μm, CyHex 100% to CyHex/EtOAc 00:100) to obtain Example 259 (363 mg, 55%) as an orange solid.

¹H-NMR (DMSO-d₆, 300 MHz) δ: 1.20-1.34 (m, 10H, 2CH₂CH₂CH₂C(O) C(CH₃)₂); 1.45-1.53 (m, 10H, 2CH₂CH₂C(O) C(CH₃)₂); 2.08-2.17 (m, 2H, 2CHₐH_{b}); 2.23-2.39 (m, 6H, 2CHₐH_{b} 2CH₂C(O)); 3.48-3.66 (m, 6H, N—CH₂N—CHₐH_{b} OCH₃); 3.68-3.81 (m, 3H, N—CH₂N—CHₐH_{b}); 4.37-4.45 (m, 2H, N—CH₂); 7.58-7.65 (m, 1H, Ar); 7.76-7.83 (m, 1H, Ar); 8.05-8.14 (m, 1H, Ar); 8.20-8.23 (m, 1H, Ar).

M/Z (M[³⁵Cl]+H)⁺: 628.5.

### Example 260: 8-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-8-oxooctanoic acid

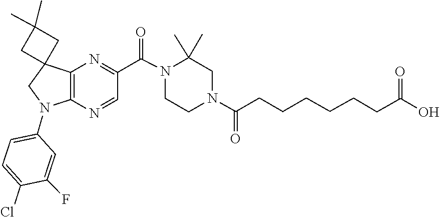 [SMILES: CC1(C)CC2(CN(c3ccc(Cl)c(F)c3)c3ncc(C(=O)N4CCN(C(=O)CCCCCCC(=O)O)CC4(C)C)nc32)C1]

To a solution of Example 259 (310 mg) in THE (4 mL) was added LiOH (62 mg) in water (2 mL). The mixture was stirred at 25° C. for 18 hours then concentrated under reduced pressure. The residue was diluted in water (20 mL), treated with HCl (1M, aq) to pH=3 and extracted with EtOAc (2*30 mL). The combined organic layers were dried over MgSO₄ and concentrated under reduced pressure. The residue was freeze dried with water to obtain Example 260 (298 mg, 98%) as a yellow solid.

¹H-NMR (DMSO-d₆, 300 MHz) δ: 1.22-1.34 (m, 10H, 2CH₂CH₂CH₂C(O) C(CH₃)₂); 1.47-1.51 (m, 10H, 2CH₂CH₂C(O) C(CH₃)₂); 2.08-2.39 (m, 8H, 4CHₐH_{b} 2CH₂C(O)); 3.48-3.81 (m, 6H, 2N—CH₂N—CHₐH_{b}); 4.37-4.45 (m, 2H, N—CH₂); 7.59-7.65 (m, 1H, Ar); 7.76-7.81 (m, 1H, Ar); 8.01-8.12 (m, 1H, Ar); 8.20-8.23 (m, 1H, Ar); 11.98 (bs, 1H, COOH).

M/Z (M[³⁵Cl]+H)⁺: 614.5.

### Example 261: N-(37-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-3-methyl-4,17,30,37-tetraoxo-7,10,13,20,23,26-hexaoxa-3,16,29-triazaheptatriacontyl)-N-methylpalmitamide

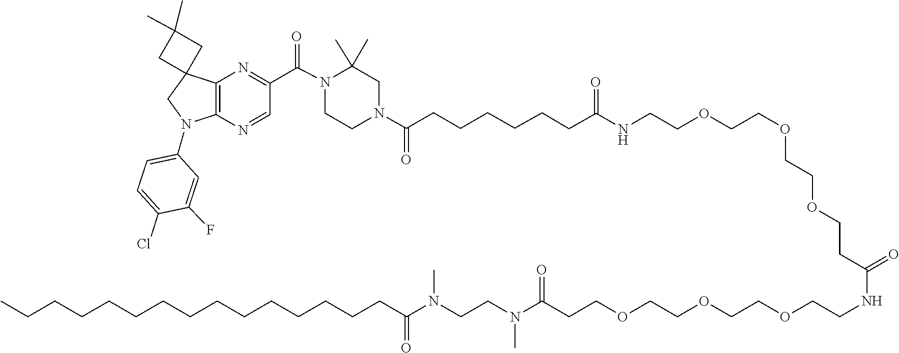 [SMILES: CCCCCCCCCCCCCCCC(=O)N(C)CCN(C)C(=O)CCOCCOCCOCCNC(=O)CCOCCOCCOCCNC(=O)CCCCCCC(=O)N1CCN(C(=O)c2cnc3c(n2)C2(CN3c3ccc(Cl)c(F)c3)CC(C)(C)C2)C(C)(C)C1]

To a solution of Example 260 (100 mg) in THE (4 mL) was added HATU (108 mg) and NEt(iPr)₂ (113 μL), after 5 minutes of stirring, N-(1-amino-26-methyl-12,25-dioxo-3,6,9,16,19,22-hexaoxa-13,26-diazaoctacosan-28-yl)-N-methylpalmitamide (163 mg) was added and the mixture stirred for 2 hours at 25° C. The mixture was treated with HCl (1M, aq, 20 mL) and extracted with EtOAc (2*30 mL). The combined organic layers were washed with NaHCO₃ (sat. aq. 20 mL), with brine (20 mL), dried over MgSO₄ and concentrated under reduced pressure. The residue was purified by flash chromatography (Interchim® 20 μm, DCM 100% to DCM/MeOH 90:10) then freeze-dried with water to obtain Example 261 (186 mg, 86%) as a white solid.

¹H-NMR (DMSO-d₆, 300 MHz) δ: 0.84 (t, =6.6 Hz, 3H, CH₂CH₃); 1.17-1.33 (m, 36H); 1.38-1.56 (m, 12H); 2.00-2.38 (m, 12H); 2.78-2.83 (m, 2H); 2.90-2.97 (m, 4H); 3.13-3.22 (m, 4H); 3.35-3.42 (m, 8H); 3.43-3.53 (m, 16H); 3.54-3.65 (m, 7H); 3.68-3.82 (m, 3H); 4.38-4.44 (m, 2H); 7.58-7.64 (m, 2H, Ar C(O)NH); 7.88-7.92 (m, 1H, NH); 8.06-8.15 (m, 1H, Ar); 8.20-8.24 (m, 1H, Ar).

M/Z (M+2H)²+: 665.3. Mp: ND.

### Compound 342: methyl 4-chloro-2,6-dimethylnicotinate

A solution of methyl 3-aminocrotonate (3.55 g) in POCl₃ (11 mL) was heated at 110° C. for 1 hour, then the reaction mixture was quenched in ice (100 mL), treated by slow addition of NaOH (15% aq. 100 mL) at 0° C., then extracted with EtOAc (2*100 mL). The combined organic layers were washed with brine (50 mL), dried over MgSO₄ and concentrated under reduced pressure. The crude was purified by flash chromatography (Interchim® 50 μm, CyHex 100% to CyHex/EtOAc 50:50) to obtain Compound 342 (1.15 g, 34%) as a yellow oil.

¹H NMR (DMSO-d₆, 300 MHz) δ: 2.42 (s, 3H, ArCH₃); 2.46 (s, 3H, ArCH₃); 3.90 (s, 3H, OCH₃); 7.41 (s, 1H, Ar).

M/Z (M[³⁵Cl]+H)⁺: 200.2.

### Compound 343: methyl 4-(3,3-dimethylpiperazin-1-yl)-2,6-dimethylnicotinate

To a solution of Compound 342 (400 mg) in MeTHF (4 mL) was added Sodium tert-butoxide (385 mg) and 2,2-dimethylpiperazine (275 mg). The mixture was sparged with Argon for 10 minutes then RuPhos Pd G4 (85 mg) was added and the mixture was stirred at 25° C. for 18 hours then HCl (1N aq., 10 mL) and EtOAc (10 mL) were added.

The mixture was filtered over a pad of Celite, the pad was rinsed with EtOAc (10 mL) and water (10 mL). The aqueous layer was washed with EtOAc (2*50 mL) then treated with NaOH (1N aq.) to pH=3, washed with DCM (2*50 mL) then further treated with NaOH (1N aq.) to pH=12. and extracted with DCM (2*60 mL). The combined organic layers were dried over MgSO₄ and concentrated under reduced pressure to obtain Compound 343 (73 mg, 13%) as a yellow oil.

M/Z (M+H)⁺: 278.3.

### Compound 344: 5-(4-chloro-3-fluorophenyl)-7,7-dimethyl-6,7-dihydro-5H-pyrrolo[2,3-b]pyrazine-2-carboxylic acid

In the first chamber of a two-chamber reactor (2*5 mL) was prepared a solution of Compound 128 (203 mg) and triethyl amine (159 μL) in a dioxane/water mixture (1:1, 3 mL). In the second chamber a suspension of Mo(CO)₆ (75 mg) in dioxane (3 mL) was prepared. Both chambers were sparged with argon for 10 minutes then XantPhos Pd G4 (11 mg) was added in the first chamber, and DBU (127 μL) was added in the second chamber. The set up was crimped and heated at 85° C. for 20 hours. The mixture in the first chamber was filtered over a Celite pad, rinsed with EtOAc (50 mL) and water (20 mL), the filtrate was washed with water (2*30 mL). The aqueous washing were acidified with HCl (1N aq.) to pH=6, the solid was recovered by filtration and dried under high vacuum, to obtain Compound 344 (100 mg, 55%) as a beige solid.

M/Z (M[³⁵Cl]+H)⁺: 322.2.

### Example 262: methyl 4-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)-2,6-dimethylnicotinate

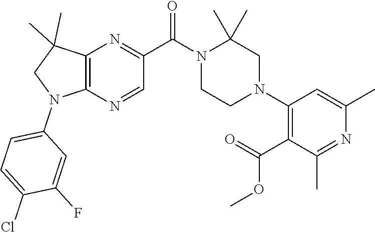 [SMILES: COC(=O)c1c(N2CCN(C(=O)c3cnc4c(n3)C(C)(C)CN4c3ccc(Cl)c(F)c3)C(C)(C)C2)cc(C)nc1C]

Example 262 was prepared according to general procedure (VIIIa) starting from Compound 344 (75 mg), Compound 343 (1.1 eq.) and N,N-diisopropylethylamine (3.0 eq.) in DCM. The crude Example 262 (256 mg) was directly engaged in the following step.

M/Z (M[³⁵Cl]+H)⁺: 581.4.

### Example 263: 4-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)-2,6-dimethylnicotinic acid

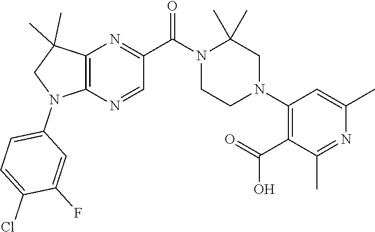 [SMILES: Cc1cc(N2CCN(C(=O)c3cnc4c(n3)C(C)(C)CN4c3ccc(Cl)c(F)c3)C(C)(C)C2)c(C(=O)O)c(C)n1]

Example 263 was prepared according to general procedure (VII) starting from crude Example 262 (246 mg). The crude was purified by preparative HPLC (Column B, H₂O+0.1% HCOOH/MeCN+0.1% HCOOH 65:35 to 00:100) and freeze dried with water to obtain Example 263 (6 mg, 5% over 2 steps) as a white solid.

¹H-NMR (DMSO-d₆, 300 MHz) δ: 1.38 (s, 6H, C(CH₃)₂); 1.50 (s, 6H, C(CH₃)₂); 2.36-2.48 (m, 6H, 2ArCH₃); 3.65-3.78 (m, 6H, 3N—CH₂); 3.98 (s, 2H, N—CH₂); 6.67 (m, 1H, Ar); 7.58-7.64 (m, 1H, Ar); 7.70-7.74 (m, 1H, Ar); 8.06-8.11 (m, 1H, Ar); 8.22 (s, 1H, Ar), 13.01 (bs, 1H, COOH).

M/Z (M[³⁵Cl]+H)⁺: 567.4.

### Compound 345: 2,6-dichloro-N-(4-chloro-3-fluorophenyl)pyridin-3-amine

A solution of 3-bromo-2,6-dichloropyridine (200 mg), 4-chloro-3-fluoroaniline (128 mg), and Sodium tert-butoxide (102 mg) in Toluene (4.5 mL) was sparged with Argon for 10 minutes, then P(t-Bu)₃ Pd G4 (26 mg) was added and the mixture was heated at 80° C. for 4 hours. The mixture was filtered over a pad of Celite, the pad was rinsed with EtOAc (10 mL) and the filtrate was concentrated under reduced pressure. The residue was dissolved in EtOAc (30 mL), washed with water (2*20 mL), with brine (20 mL) and dried over MgSO₄ and concentrated under reduced pressure to obtain crude Compound 345 (298 mg) as a brown oil which was directedly engaged in the following step.

M/Z (M[³⁵Cl]₂[³⁷Cl]+H)⁺: 293.1.

### Compound 346: 6-chloro-1-(4-chloro-3-fluorophenyl)-3,3-dimethyl-2,3-dihydro-1H-pyrido[2,3-b][1,4]oxazine

To a solution of crude Compound 345 (257 mg) and Sodium tert-butoxide (102 mg) in THE (4 mL) at 50° C. were added 11 portions of 1,2-Epoxy-2-methylpropane (11*94 μL) over 7 days. The reaction mixture was quenched with water (10 mL) and extracted with EtOAc (2*10 mL). The combined organic layers were washed with brine (10 mL), dried over MgSO₄ and concentrated under reduced pressure. The residue was purified by flash chromatography (Interchim® 50 μm, CyHex 100% to CyHex/EtOAc 70:30) to obtain Compound 346 (119 mg, 41% over 2 steps) as a brown oil.

¹H-NMR (DMSO-d₆, 300 MHz) δ: 1.32 (s, 6H, C(CH₃)₂); 3.55 (s, 2H, N—CH₂); 6.91 (d, J 8.4 Hz, 1H, Ar); 7.14-7.18 (m, 1H, Ar); 7.37-7.41 (m, 1H, Ar); 7.43 (d, J 8.4 Hz, 1H, Ar), 7.52-7.58 (m, 1H, Ar).

M/Z (M[³⁵Cl]₂+H)⁺: 327.2.

### Example 264: methyl 2-(6-(4-(1-(4-chloro-3-fluorophenyl)-3,3-dimethyl-2,3-dihydro-1H-pyrido[2,3-b][1,4]oxazine-6-carbonyl)-3,3-dimethylpiperazin-1-yl)pyridin-3-yl)acetate

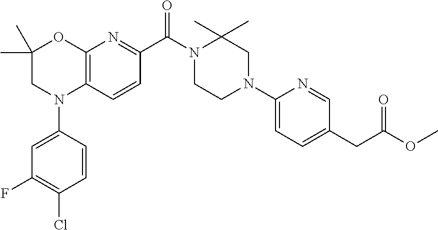

Example 264 was prepared according to general procedure (XIV) starting from Compound 346 (119 mg) and Compound 111 (1.5 eq), and using triethyl amine (4.0 eq), Mo(CO)₆ (1.5 eq), XantPhos Pd G4 (0.10 eq), and DBU (4.5 eq). The crude Example 264 (142 mg) was directly engaged in the following step.

M/Z (M[³⁵Cl]+H)⁺: 582.3.

### Example 265: 2-(6-(4-(1-(4-chloro-3-fluorophenyl)-3,3-dimethyl-2,3-dihydro-1H-pyrido[2,3-b][1,4]oxazine-6-carbonyl)-3,3-dimethylpiperazin-1-yl)pyridin-3-yl)acetic acid

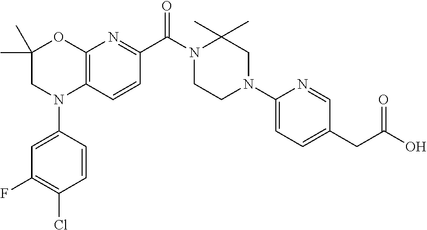 [SMILES: CC1(C)CN(c2ccc(Cl)c(F)c2)c2ccc(C(=O)N3CCN(c4ccc(CC(=O)O)cn4)CC3(C)C)nc2O1]

Example 265 was prepared according to general procedure (VII) starting from Example 264 (142 mg). The crude was purified by preparative HPLC (Column B, H₂O+0.1% HCOOH/MeCN+0.1% HCOOH 65:35 to 0:100) and freeze dried to obtain Example 265 (25 mg, 12% over 2 steps) as a white solid.

¹H-NMR (DMSO-d₆, 300 MHz) δ: 1.35 (s, 6H, C(CH₃)₂); 1.47 (s, 6H, C(CH₃)₂); 3.40-3.47 (m, 4H, N—CH₂); 3.58 (s, 2H, N—CH₂); 3.75-3.86 (m, 4H, N—CH₂, CH₂C(O)); 6.57 (d, J 8.7 Hz, 1H, Ar); 7.06 (d, J 8.2 Hz, 1H, Ar); 7.22 (dd, J 8.7, 1.9 Hz, 1H, Ar); 7.34-7.47 (m, 3H, 3Ar); 7.55-7.63 (m, 1H, Ar); 7.94 (d, J 1.9 Hz, 1H, Ar); COOH signal was not observed.

M/Z (M[³⁵Cl]+H)⁺: 568.4

### Compound 347: 2′-(benzylthio)-5′-(4-chloro-3-fluorophenyl)-3,3-dimethyl-5′,6′-dihydrospiro[cyclobutane-1,7′-pyrrolo[2,3-b]pyrazine]

A solution of Compound 307 (300 mg), phenylmethanethiol (98 μL) and Net(iPr)₂ (260 μL) in Dioxane (7.5 mL) was sparged with Argon for 5 minutes then XantPhos Pd G4 (73 mg) was added and the mixture was heated at 95° C. for 18 hours. The mixture was filtered over a pad of Celite, the pad was rinsed with EtOAc (10 mL) and the filtrate was concentrated under reduced pressure. The residue was purified by flash chromatography (Interchim® 50 μm, CyHex 100% to CyHex/EtOAc 90:10) to obtain Compound 347 (297 mg, 89%) as a yellow solid.

M/Z (M[³⁷Cl]+H)⁺: 442.1.

### Compound 348: 5′-(4-chloro-3-fluorophenyl)-3,3-dimethyl-5′,6′-dihydrospiro[cyclobutane-1,7′-pyrrolo[2,3-b]pyrazine]-2′-sulfonyl chloride

To a solution of Compound 347 (150 mg) in a water/acetic acid mixture (4.8 mL 1:11), N-chlorosuccinimide (137 mg) was added. The mixture was stirred at 25° C. for 2 hours then diluted with water (10 mL), and extracted with EtOAc (2*10 mL). The combined organic layers were concentrated under reduced pressure and the residue was co-evaporated with CyHex (3*10 mL) to obtain crude Compound 348 (193 mg) as a yellow solid which was directly engaged in the following step.

M/Z (M+H)+: Not observed.

Derivation with piperidine was used to monitor the reaction.

### Example 266: 1-(1-((5′-(4-chloro-3-fluorophenyl)-3,3-dimethyl-5′,6′-dihydrospiro[cyclobutane-1,7′-pyrrolo[2,3-b]pyrazin]-2′-yl)sulfonyl)piperidin-4-yl)imidazolidin-2-one

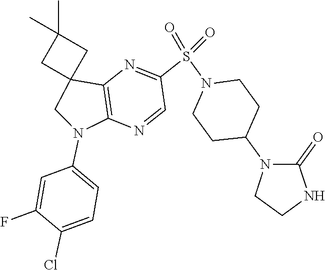 [SMILES: CC1(C)CC2(CN(c3ccc(Cl)c(F)c3)c3ncc(S(=O)(=O)N4CCC(N5CCNC5=O)CC4)nc32)C1]

To a solution of crude Compound 348 (97 mg) in DCM (2.3 mL) was added 1-(Piperidin-4-yl)imidazolidine-2-one (118 mg) and the mixture was stirred at 25° C. for 20 hours. The residue was purified by flash chromatography (Interchim® 20 μm, DCM 100% to DCM/MeOH 90:10), then by preparative HPLC (Column B, H₂O+0.1% HCOOH/MeCN+0.1% HCOOH 50:50 to 10:90) and freeze dried to obtain Example 266 (12 mg, 6% over 2 steps) as a white solid.

¹H-NMR (DMSO-d₆, 300 MHz) δ: 1.26 (s, 3H, C(CH₃)); 1.28 (s, 3H, C(CH₃)); 1.59-1.67 (m, 4H, CH₂); 2.14-2.18 (m, 2H, CHₐH_{b}); 2.34-2.38 (m, 2H, CHₐH_{b}); 2.73-2.82 (m, 2H, N—CH₂); 3.18-3.25 (m, 4H, N—CH₂); 3.47-3.57 (m, 1H, N—CH); 3.79-3.83 (m, 2H, N—CH₂); 4.44 (s, 2H, N—CH₂); 6.28 (s, 1H, NH); 7.65 (t, J 8.8 Hz, 1H, Ar); 7.79-7.83 (m, 1H, Ar); 8.12 (dd, J 12.4, 2.5 Hz, 1H, Ar); 8.38 (s, 1H, Ar).

### Compound 349: 4-(4-chloro-3-fluorophenyl)-2,2-dimethyl-3,4-dihydro-2H-pyrazino[2,3-b][1,4]oxazine

To a solution of crude 2,3-dichloropyrazine (500 mg) and 4-Chloro-3-fluoroaniline (489 mg) in THE (15 mL) was added Sodium tert-butoxide (968 mg) at 0° C. The mixture was stirred at 20° C. for 18 hours then at 50° C. were added 9 portions of 1,2-Epoxy-2-methylpropane (9*358 μL) over 5 days. The reaction mixture was diluted with EtOAc (50 mL), washed with water (2*25 mL), with brine (20 mL), dried over MgSO₄ and concentrated under reduced pressure. The residue was purified by flash chromatography (Interchim® 50 μm, CyHex 100% to CyHex/EtOAc 40:60) to obtain Compound 349 (600 mg, 61%) as a yellow oil.

¹H-NMR (DMSO-d₆, 300 MHz) δ: 1.38 (s, 6H, C(CH₃)₂); 3.76 (s, 2H, N—CH₂); 7.26-7.33 (m, 1H, Ar); 7.52-7.61 (m, 2H, 2Ar); 7.63 (d, J 3.0 Hz, 1H, Ar); 7.69 (d, J 3.0 Hz, 1H, Ar).

M/Z (M[³⁵Cl]+H)⁺: 294.2.

### Compound 350: 7-bromo-4-(4-chloro-3-fluorophenyl)-2,2-dimethyl-3,4-dihydro-2H-pyrazino[2,3-b][1,4]oxazine

Compound 350 (800 mg, 64%) was obtained as a brown oil following general procedure (XVI) in EtOAc without further purification, starting from Compound 349 (600 mg).

M/Z (M[³⁷Cl][⁸¹Br]+H)⁺: 376.1.

### Compound 351: 4-(4-chloro-3-fluorophenyl)-2,2-dimethyl-3,4-dihydro-2H-pyrazino[2,3-b][1,4]oxazine-7-carboxylic acid

In the first chamber of a two-chamber reactor (2*5 mL) was prepared a solution of Compound 350 (300 mg) and triethyl amine (224 μL) in a dioxane/water mixture (1:1, 3 mL). In the second chamber a suspension of Mo(CO)₆ (212 mg) in dioxane (3 mL) was prepared. Both chambers were sparged with argon for 10 minutes then XantPhos Pd G4 (16 mg) was added in the first chamber, and DBU (180 μL) was added in the second chamber. The set up was crimped and heated at 85° C. for 18 hours. The mixture in the first chamber was filtered over a Celite pad, rinsed with MeOH (20 mL), the filtrate was concentrated under reduced pressure then dissolved in water (30 mL). The solution was washed with EtOAc (2*25 mL), treated with HCl (1M aq., to pH=3) and extracted with MTBE (3*20 mL). The combined organic layers were dried over MgSO₄ and concentrated under reduced pressure to obtain Compound 351 (216 mg) as a brown oil.

M/Z (M[³⁵Cl]+H)⁺: 338.2.

### Example 267: methyl 2-(6-(4-(4-(4-chloro-3-fluorophenyl)-2,2-dimethyl-3,4-dihydro-2H-pyrazino[2,3-b][1,4]oxazine-7-carbonyl)-3,3-dimethylpiperazin-1-yl)266yridine-3-yl)acetate

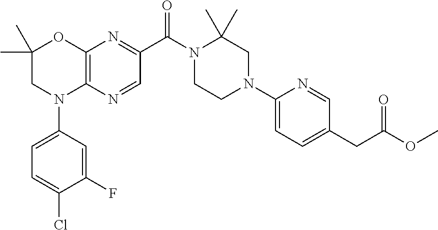

Example 267 was prepared according to general procedure (VIIIb) starting from Compound 351 (100 mg), Compound 111 (1.2 eq.) and DIPEA (3 eq.) in MeTHF. Example 267 (208 mg) was obtained without further purification as a brown oil.

M/Z (M[³⁵Cl]+H)⁺: 583.3

### Example 268: 2-(6-(4-(4-(4-chloro-3-fluorophenyl)-2,2-dimethyl-3,4-dihydro-2H-pyrazino[2,3-b][1,4]oxazine-7-carbonyl)-3,3-dimethylpiperazin-1-yl)266yridine-3-yl)acetic acid

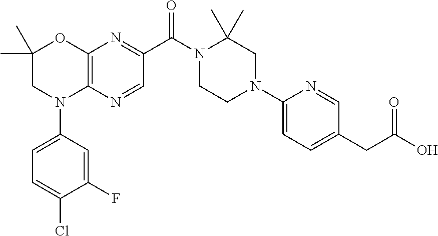 [SMILES: CC1(C)CN(c2ccc(Cl)c(F)c2)c2ncc(C(=O)N3CCN(c4ccc(CC(=O)O)cn4)CC3(C)C)nc2O1]

Example 268 was prepared according to general procedure (VII) starting from Example 267 (208 mg). The crude was purified by preparative HPLC (Column B, H₂O+0.1% HCOOH/MeCN+0.1% HCOOH 80:20 to 40:60) and freeze dried with HCl (0.1N, aq.) to obtain Example 268 (108 mg, 47% over 3 steps) as a yellow solid.

¹H-NMR (DMSO-d₆, 300 MHz) δ: 1.41 (s, 6H, (CH₃)₂); 1.52 (s, 6H, (CH₃)₂); 3.64-3.71 (m, 4H, 2N—CH₂); 3.83 (s, 2H, CH₂C(O)); 3.90-3.94 (m, 2H, N—CH₂); 3.98 (s, 2H, N—CH₂); 7.32-7.39 (m, 2H, 2Ar); 7.57-7.64 (m, 2H, 2Ar); 7.94-8.00 (m, 3H, 3Ar); COOH signal was not observed.

M/Z (M[³⁵Cl]+H)⁺: 569.4

### Compound 352: 5-(tert-butyl) 2-methyl 7,7-dimethyl-6,7-dihydrothiazolo[5,4-c]pyridine-2,5(4H)-dicarboxylate

To a solution of tert-butyl 2-bromo-7,7-dimethyl-6,7-dihydrothiazolo[5,4-c]pyridine-5(4H)-carboxylate (754 mg) in MeOH (30 mL) was added Pd(OAc)₂ (108 mg), X-Phos (310 mg), and NEt₃ (1.20 mL). The mixture was heated at 60° C. for 5 hours under CO atmosphere then filtered and the filtrate was concentrated under reduced pressure. The residue was purified by flash chromatography (Petroleum Ether 100% to Petroleum Ether/EtOAc 75:25) to obtain Compound 352 (283 mg, 39%) as a brown oil.

M/Z (M+H)⁺: 327.2.

### Compound 353: methyl 7,7-dimethyl-4,5,6,7-tetrahydrothiazolo[5,4-c]pyridine-2-carboxylate

To a solution of Compound 352 (253 mg) in DCM (3 mL) was added TFA (1.0 mL) at 0° C. The mixture was stirred at 25° C. for 2 hours then diluted with EtOAc (50 mL) and washed with NaHCO₃ (sat. aq. 3*20 mL). Combined aqueous washes were adjusted to pH=11 and extracted with EtOAc (3*30 mL). All organic layers were combined, dried over Na₂SO₄ and concentrated under reduced pressure to obtain crude Compound 353 (181 mg) as a brown solid.

M/Z (M+H)⁺: 227.2.

### Compound 354: methyl 5-(4-chloro-3-fluorophenyl)-7,7-dimethyl-4,5,6,7-tetrahydrothiazolo[5,4-c]pyridine-2-carboxylate

To a solution of Compound 353 (151 mg) in Dioxane (15 mL) was added Cs₂CO₃ (437 mg) and 1-chloro-2-fluoro-4-iodobenzene (256 mg) was added RuPhos Pd G4 (57 mg). The mixture was heated at 100° C. for 4 hours under inert atmosphere then filtered and the filtrate was concentrated under reduced pressure. The residue was purified by flash chromatography (Petroleum Ether 100% to Petroleum Ether/EtOAc 80:20) to obtain Compound 354 (151 mg, 64%) as a yellow solid.

M/Z (M[³⁵Cl]+H)⁺: 355.2.

¹H-NMR (DMSO-d₆, 400 MHz) δ: 1.31 (s, 6H, C(CH₃)₂); 3.46 (s, 2H, N—CH₂); 3.90 (s, 3H, OCH₃); 4.59 (s, 2H, N—CH₂); 6.86-6.91 (m, 1H, Ar); 7.02-7.09 (m, 1H, Ar); 7.35-7.41 (m, 1H, Ar).

### Compound 355: 5-(4-chloro-3-fluorophenyl)-7,7-dimethyl-4,5,6,7-tetrahydrothiazolo[5,4-c]pyridine-2-carboxylic acid

To a solution of Compound 354 (152 mg) in THE (15 mL) was added LiOH (103 mg) in water (2 mL) at 0° C. The mixture was heated at 60° C. for 3 hours then treated with HCl (2M, aq.) to pH=3 and extracted with EtOAc (3*30 mL). The combined organic layers were washed with brine (40 mL) dried over Na₂SO₄ and concentrated under reduced pressure to obtain crude Compound 355 (174 mg) as a brown oil.

M/Z (M[³⁵Cl]+H)⁺: 341.0.

### Example 269: methyl 2-(6-(4-(5-(4-chloro-3-fluorophenyl)-7,7-dimethyl-4,5,6,7-tetrahydrothiazolo[5,4-c]pyridine-2-carbonyl)-3,3-dimethylpiperazin-1-yl)pyridin-3-yl)acetate

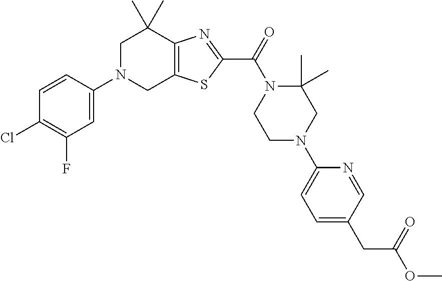 [SMILES: COC(=O)Cc1ccc(N2CCN(C(=O)c3nc4c(s3)CN(c3ccc(Cl)c(F)c3)CC4(C)C)C(C)(C)C2)nc1]

To a solution of Compound 355 (119 mg) in DMF (8 mL) was added diethyl (4-oxobenzo[d][1,2,3]triazin-3(4H)-yl) phosphate (209 mg) and NEt(iPr)₂ (0.30 mL) and the mixture was stirred at 0° C. for 30 minutes then Compound 111 (110 mg) was added. The mixture was stirred at 25° C. for 5 hours then diluted with water (20 mL) and extracted with EtOAc (3*30 mL). The combined organic layers were washed with brine (30 mL) dried over Na₂SO₄ and concentrated under reduced pressure. The residue was purified by flash chromatography (Petroleum Ether 100% to Petroleum Ether/EtOAc 60:40) to obtain Example 269 (75 mg, 37%) as a yellow solid.

M/Z (M[³⁵Cl]+H)⁺: 586.2.

### Example 270: 2-(6-(4-(5-(4-chloro-3-fluorophenyl)-7,7-dimethyl-4,5,6,7-tetrahydrothiazolo[5,4-c]pyridine-2-carbonyl)-3,3-dimethylpiperazin-1-yl)pyridin-3-yl)acetic acid

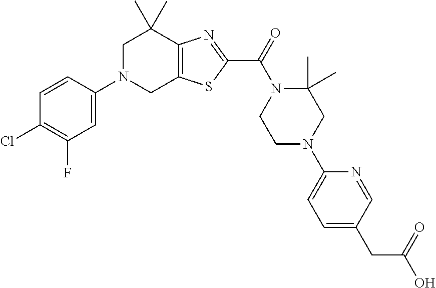 [SMILES: CC1(C)CN(c2ccc(Cl)c(F)c2)Cc2sc(C(=O)N3CCN(c4ccc(CC(=O)O)cn4)CC3(C)C)nc21]

To a solution of Example 269 (103 mg) in THE (10 mL) was added LiOH (43 mg) in water (2 mL) at 0° C. The mixture was stirred at 25° C. for 3 hours then treated with HCl (2M aq.) to pH=2 and extracted with EtOAc (3*20 mL). The combined organic layers were washed with brine (40 mL) dried over Na₂SO₄ and concentrated under reduced pressure. The residue was purified by preparative HPLC (H₂O/MeCN+0.05% HCOOH) and further purified by flash chromatography (C18 as stationary phase, water 100% to MeOH/water 25:75) to obtain Example 270 (28 mg, 28%) as a yellow solid.

¹H NMR (DMSO-d₆, 400 MHz) δ: 1.31 (s, 6H, C(CH₃)₂); 1.47 (s, 6H, C(CH₃)₂); 3.22 (s, 2H, N—CH₂); 3.44 (s, 2H, N—CH₂); 3.50-3.56 (m, 2H, N—CH₂); 3.88 (s, 2H, CH₂C(O)); 4.52-4.54 (m, 4H, N—CH₂); 6.52-6.56 (m, 1H, Ar); 6.85-6.89 (m, 1H, Ar); 7.02-7.08 (m, 1H, Ar); 7.41-7.45 (m, 1H, Ar); 7.93 (d, J 1.6 Hz, 1H, Ar); COOH was not observed.

M/Z (M[³⁵Cl]+H)⁺: 572.2.

### Example 271: tert-butyl 2-(4-(5-(2-methoxy-2-oxoethyl)pyridin-2-yl)-2,2-dimethylpiperazine-1-carbonyl)-7,7-dimethyl-2,4,6,7-tetrahydro-5H-pyrazolo[4,3-c]pyridine-5-carboxylate

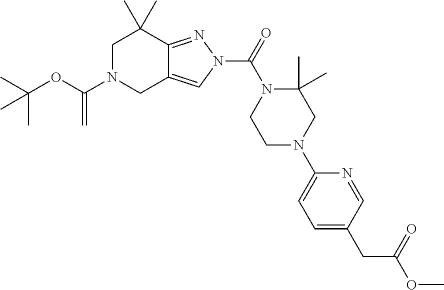 [SMILES: C=C(OC(C)(C)C)N1Cc2cn(C(=O)N3CCN(c4ccc(CC(=O)OC)cn4)CC3(C)C)nc2C(C)(C)C1]

To a solution of tert-butyl 7,7-dimethyl-1,4,6,7-tetrahydro-5H-pyrazolo[4,3-c]pyridine-5-carboxylate (100 mg) in DCM (10 mL) was added triphosgene (59 mg), and the mixture was stirred at 25° C. for 5 minutes then NEt(iPr)₂ (0.43 mL) was added dropwise at 0° C. The mixture was stirred at 25° C. for 2 hours then diluted with DCM (20 mL), quenched with water (20 mL) and extracted with DCM (3*20 mL). The combined organic layers were washed with brine (30 mL) dried over Na₂SO₄ and concentrated under reduced pressure. The residue was purified by flash chromatography (Petroleum Ether 100% to Petroleum Ether/EtOAc 65:35) to obtain Example 271 (106 mg, 49%) as a white solid.

¹H NMR (DMSO-d₆, 400 MHz) δ: 1.22 (s, 6H, C(CH₃)₂); 1.43 (s, 9H, C(CH₃)₃); 1.45 (s, 6H, C(CH₃)₂); 3.39 (s, 2H, N—CH₂); 3.53-3.54 (m, 4H, 2N—CH₂); 3.61 (s, 3H, OCH₃); 3.86 (s, 2H, CH₂C(O)); 3.87-3.94 (m, 2H, N—CH₂); 4.45 (s, 2H, N—CH₂); 6.63 (d, J 8.8 Hz, 1H, Ar); 7.45 (d, J 8.8 2.0 Hz, 1H, Ar); 7.97 (d, J 2.0 Hz, 1H, Ar);

M/Z (M+H)⁺: 541.4.

### Compound 356: methyl 2-(6-(4-(7,7-dimethyl-4,5,6,7-tetrahydro-2H-pyrazolo[4,3-c]pyridine-2-carbonyl)-3,3-dimethylpiperazin-1-yl)pyridin-3-yl)acetate

To a solution of Example 271 (182 mg) in DCM (3 mL) was added TFA (1.0 mL) at 0° C. and the mixture was stirred at 25° C. for 3 hours. The mixture was concentrated under reduced pressure, the residue was diluted with NaHCO₃ (sat. aq. 30 mL) and extracted with EtOAc (5*30 mL). The combined organic layers were dried over Na₂SO₄ and concentrated under reduced pressure. To obtain crude Compound 356 (131 mg) as a yellow solid.

M/Z (M+H)⁺: 441.4.

### Example 272: methyl 2-(6-(4-(5-(4-chloro-3-fluorophenyl)-7,7-dimethyl-4,5,6,7-tetrahydro-2H-pyrazolo[4,3-c]pyridine-2-carbonyl)-3,3-dimethylpiperazin-1-yl)pyridin-3-yl)acetate

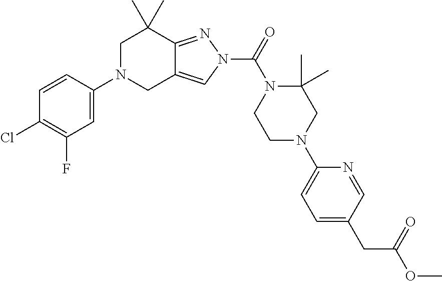 [SMILES: COC(=O)Cc1ccc(N2CCN(C(=O)n3cc4c(n3)C(C)(C)CN(c3ccc(Cl)c(F)c3)C4)C(C)(C)C2)nc1]

To a solution of Compound 356 (123 mg) in Dioxane (10 mL) was added Cs₂CO₃ (183 mg) and 1-chloro-2-fluoro-4-iodobenzene (108 mg) was added RuPhos Pd G4 (24 mg). The mixture was heated at 100° C. for 4 hours under inert atmosphere then filtered and the filtrate was concentrated under reduced pressure. The residue was purified by flash chromatography (Petroleum Ether 100% to Petroleum Ether/EtOAc 60:40) to obtain Example 272 (104 mg, 65%) as a yellow solid.

M/Z (M[³⁵Cl]+H)⁺: 569.2.

### Example 273: 2-(6-(4-(5-(4-chloro-3-fluorophenyl)-7,7-dimethyl-4,5,6,7-tetrahydro-2H-pyrazolo[4,3-c]pyridine-2-carbonyl)-3,3-dimethylpiperazin-1-yl)pyridin-3-yl)acetic acid

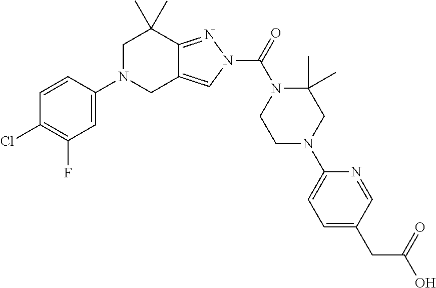 [SMILES: CC1(C)CN(c2ccc(Cl)c(F)c2)Cc2cn(C(=O)N3CCN(c4ccc(CC(=O)O)cn4)CC3(C)C)nc21]

To a solution of Example 272 (110 mg) in THE (10 mL) was added LiOH (47 mg) in water (2 mL) at 0° C. The mixture was stirred at 25° C. for 3 hours then treated with HCl (2M aq.) to pH=3 and extracted with EtOAc (3*20 mL). The combined organic layers were washed with brine (40 mL) dried over Na₂SO₄ and concentrated under reduced pressure. The residue was purified by preparative HPLC (H₂O/MeCN+0.05% HCOOH) to obtain Example 273 (44 mg, 41%) as a white solid.

¹H NMR (DMSO-d₆, 400 MHz) δ: 1.31 (s, 6H, C(CH₃)₂); 1.46 (s, 6H, C(CH₃)₂); 3.37 (s, 2H, N—CH₂); 3.42 (s, 2H, N—CH₂); 3.50-3.59 (m, 2H, N—CH₂); 3.85 (s, 2H, CH₂C(O)); 3.88-3.99 (m, 2H, N—CH₂); 4.27 (s, 2H, N—CH₂); 6.60-6.64 (m, 1H, Ar); 6.81-6.85 (m, 1H, Ar); 6.97-7.03 (m, 1H, Ar); 7.34-7.36 (m, 1H, Ar); 7.42-7.46 (m, 1H, Ar); 7.92 (s, 1H, Ar); 7.95 (d, J 2.0 Hz, 1H, Ar); 12.35 (bs, 1H, COOH).

M/Z (M[³⁵Cl]+H)⁺: 555.2.

### Compound 357: 5-chloro-1-(3-fluoro-4-(trifluoromethyl)phenyl)-3,3-dimethyl-2,3-dihydro-1H-pyrrolo[3,2-b]pyridine

Compound 357 was prepared according to general procedure (X) starting from Compound 70 (300 mg) and 4-bromo-2-fluoro-1-(trifluoromethyl)benzene (2.0 eq) with ^{t}BuONa (3.0 eq) at 100° C. for 16 hours. The crude was purified by flash chromatography (Interchim® 50 μm, CyHex 100% to CyHex/EtOAc 50:50) to obtain Compound 357 (110 mg, 19%) as a white solid.

M/Z (M[³⁵Cl]+H)⁺: 345.2

### Example 274: methyl 6-(4-(1-(3-fluoro-4-(trifluoromethyl)phenyl)-3,3-dimethyl-2,3-dihydro-1H-pyrrolo[3,2-b]pyridine-5-carbonyl)-3,3-dimethylpiperazin-1-yl)-2,4-dimethylnicotinate

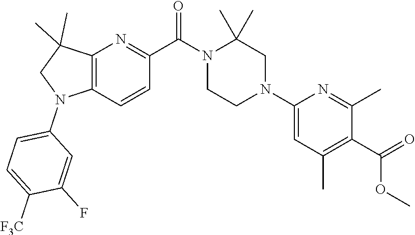 [SMILES: COC(=O)c1c(C)cc(N2CCN(C(=O)c3ccc4c(n3)C(C)(C)CN4c3ccc(C(F)(F)F)c(F)c3)C(C)(C)C2)nc1C]

Example 274 was prepared according to general procedure (XIV) starting from Compound 357 (110 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 20 μm, CyHex 100% to CyHex/EtOAc 40:60) to obtain Example 274 (55 mg) as a beige solid.

M/Z (M+H)⁺: 615.1.

### Example 275: 6-(4-(1-(3-fluoro-4-(trifluoromethyl)phenyl)-3,3-dimethyl-2,3-dihydro-1H-pyrrolo[3,2-b]pyridine-5-carbonyl)-3,3-dimethylpiperazin-1-yl)-2,4-dimethylnicotinic acid

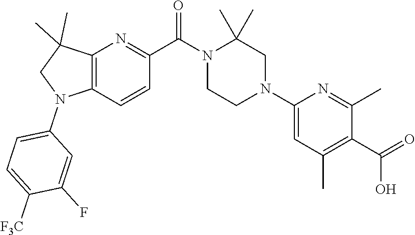 [SMILES: Cc1cc(N2CCN(C(=O)c3ccc4c(n3)C(C)(C)CN4c3ccc(C(F)(F)F)c(F)c3)C(C)(C)C2)nc(C)c1C(=O)O]

Example 275 was prepared according to general procedure (XI) starting from Example 274 (55 mg). The crude was purified by preparative HPLC (Column B, H₂O+0.1% HCOOH/MeCN+0.1% HCOOH 65:35 to 00:100) and freeze dried with HCl (0.1N in water) to obtain Example 275 (9 mg, 16% over 2 steps) as a yellow solid.

¹H-NMR (DMSO-d₆, 300 MHz) δ: 1.37 (s, 6H, C(CH₃)₂); 1.53 (s, 6H, C(CH₃)₂); 2.34 (s, 3H, ArCH₃); 2.54 (s, 3H, ArCH₃); 3.62-3.71 (m, 2H, N—CH₂); 3.86-3.91 (m, 2H, N—CH₂); 3.92-3.97 (m, 4H, 2N—CH₂); 6.72 (bs, 1H, Ar); 7.27-7.36 (m, 2H, 2Ar); 7.41 (d, J 8.7 Hz, 1H, Ar); 7.69-7.76 (m, 2H, 2Ar); COOH was not observed.

M/Z (M+H)⁺: 601.0.

### Example 276: methyl 6-(4-(7,7-dimethyl-5-(3,4,5-trifluorophenyl)-6,7-dihydro-5H-pyrrolo[2,3-b]pyrazine-2-carbonyl)-3,3-dimethylpiperazin-1-yl)-2,4-dimethylnicotinate

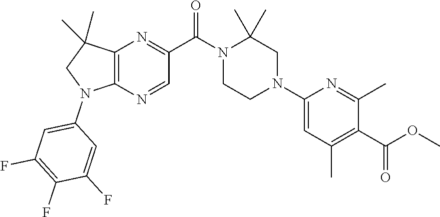 [SMILES: COC(=O)c1c(C)cc(N2CCN(C(=O)c3cnc4c(n3)C(C)(C)CN4c3cc(F)c(F)c(F)c3)C(C)(C)C2)nc1C]

Example 276 was prepared according to general procedure (XIV) starting from Compound 276 (150 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 50:50) to obtain Example 276 (147 mg, 60%) as a yellow solid.

¹H-NMR (DMSO-d₆, 300 MHz) δ: 1.40 (s, 6H, C(CH₃)₂); 1.50 (s, 6H, C(CH₃)₂); 2.23 (s, 3H, ArCH₃); 2.34 (s, 3H, ArCH₃); 3.53-3.56 (m, 2H, N—CH₂); 3.75-3.80 (m, 5H, OCH₃N—CH₂); 3.88-3.90 (m, 2H, N—CH₂); 3.97 (s, 2H, N—CH₂); 6.37 (s, 1H, Ar); 7.87-7.93 (m, 2H, 2Ar); 8.23 (s, 1H, Ar).

M/Z (M+H)⁺: 583.5

### Example 277: 6-(4-(7,7-dimethyl-5-(3,4,5-trifluorophenyl)-6,7-dihydro-5H-pyrrolo[2,3-b]pyrazine-2-carbonyl)-3,3-dimethylpiperazin-1-yl)-2,4-dimethylnicotinic acid

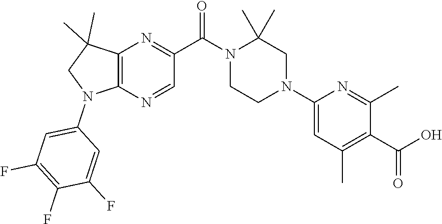 [SMILES: Cc1cc(N2CCN(C(=O)c3cnc4c(n3)C(C)(C)CN4c3cc(F)c(F)c(F)c3)C(C)(C)C2)nc(C)c1C(=O)O]

Example 277 was prepared according to general procedure (XI) starting from Example 276 (147 mg). The crude was purified by preparative HPLC (Column A, H₂O+0.1% HCOOH/MeCN+0.1% HCOOH 70:30 to 30:70) and freeze dried with HCl (0.1N in water) to obtain Example 277 (52 mg, 34%) as a yellow solid.

¹H-NMR (DMSO-d₆, 300 MHz) δ: 1.39 (s, 6H, C(CH₃)₂); 1.53 (s, 6H, C(CH₃)₂); 2.33 (s, 3H, ArCH₃); 3.62-3.71 (m, 2H, N—CH₂); 3.83-3.88 (m, 2H, N—CH₂); 3.94-3.97 (m, 4H, 2N—CH₂); 6.72 (bs, 1H, Ar); 7.88-7.93 (m, 2H, 2Ar); 8.25 (s, 1H, Ar); One ArCH₃ and COOH were not observed.

M/Z (M+H)⁺: 569.4

### Compound 358: 5-chloro-1-(4-cyclopropylphenyl)-3,3-dimethyl-2,3-dihydro-1H-pyrrolo[3,2-b]pyridine

Compound 358 was prepared according to general procedure (X) starting from Compound 70 (150 mg) and 1-bromo-4-cyclopropylbenzene (2.0 eq) with ^{t}BuONa (3.0 eq) at 100° C. for 16 hours. The crude was purified by flash chromatography (Interchim® 50 μm, CyHex 100% to CyHex/EtOAc 40:60) to obtain Compound 357 (159 mg, 65%) as a beige solid.

M/Z (M[³⁵Cl]+H)⁺: 299.6

### Example 278: methyl 6-(4-(1-(4-cyclopropylphenyl)-3,3-dimethyl-2,3-dihydro-1H-pyrrolo[3,2-b]pyridine-5-carbonyl)-3,3-dimethylpiperazin-1-yl)-2,4-dimethylnicotinate

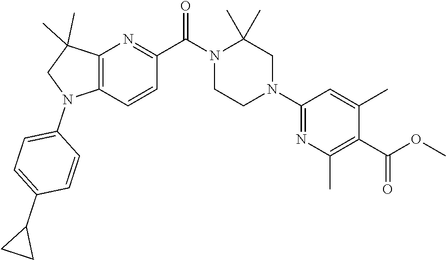 [SMILES: COC(=O)c1c(C)cc(N2CCN(C(=O)c3ccc4c(n3)C(C)(C)CN4c3ccc(C4CC4)cc3)C(C)(C)C2)nc1C]

Example 278 was prepared according to general procedure (XIV) starting from Compound 358 (110 mg) and Compound 10 (2.0 eq), and using triethyl amine (4.0 eq), Mo(CO)₆ (1.5 eq), XantPhos Pd G4 (0.10 eq), and DBU (4.5 eq). The crude was purified by flash chromatography (Interchim® 50 μm, CyHex 100% to CyHex/EtOAc 50:50) to obtain Example 278 (170 mg) as a beige solid.

¹H-NMR (DMSO-d₆, 300 MHz) δ: 0.60-0.65 (m, 2H, CH—CH₂); 0.89-0.95 (m, 2H, CH—CH₂); 1.35 (s, 6H, C(CH₃)₂); 1.50 (s, 6H, C(CH₃)₂); 1.85-1.98 (m, 1H, CH—CH₂); 2.24 (s, 3H, ArCH₃); 2.34 (s, 3H, ArCH₃); 3.52-3.61 (m, 2H, N—CH₂); 3.78 (s, 3H, OCH₃); 3.80-3.88 (m, 6H, 3N—CH₂); 6.36 (s, 1H, Ar); 7.11 (d, J 8.7 Hz, 2H, 2Ar); 7.19 (d, J 8.7 Hz, 2H, 2 Ar); 7.29-7.36 (m, 2H, 2Ar).

M/Z (M+H)⁺: 569.0

### Example 279: 6-(4-(1-(4-cyclopropylphenyl)-3,3-dimethyl-2,3-dihydro-1H-pyrrolo[3,2-b]pyridine-5-carbonyl)-3,3-dimethylpiperazin-1-yl)-2,4-dimethylnicotinic acid

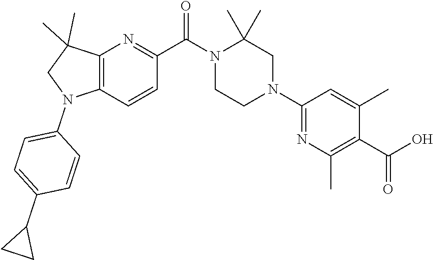 [SMILES: Cc1cc(N2CCN(C(=O)c3ccc4c(n3)C(C)(C)CN4c3ccc(C4CC4)cc3)C(C)(C)C2)nc(C)c1C(=O)O]

Example 279 was prepared according to general procedure (XI) starting from Example 278 (85 mg). The crude was purified by preparative HPLC (Column A, H₂O+0.1% HCOOH/MeCN+0.1% HCOOH 70:30 to 30:70) and freeze dried with HCl (0.1N in water) to obtain Example 279 (45 mg, 54% over 2 steps) as a white solid.

¹H-NMR (DMSO-d₆, 300 MHz) δ: 0.61-0.66 (m, 2H, CH—CH₂); 0.89-0.96 (m, 2H, CH—CH₂); 1.35 (s, 6H, C(CH₃)₂); 1.49 (s, 6H, C(CH₃)₂); 1.86-1.95 (m, 1H, CH—CH₂); 2.28 (s, 3H, ArCH₃); 2.40 (s, 3H, ArCH₃); 3.54-3.60 (m, 2H, N—CH₂); 3.81 (s, 2H, N—CH₂); 3.84-3.93 (m, 4H, 2N—CH₂); 6.40 (s, 1H, Ar); 7.11 (d, J 8.7 Hz, 2H, 2Ar); 7.19 (d, J 8.7 Hz, 2H, 2 Ar); 7.32-7.35 (m, 2H, 2Ar); COOH was not observed.

M/Z (M+H)⁺: 555.0.

### Compound 359: tert-butyl 5-bromo-3,3-dimethyl-4-oxopiperidine-1-carboxylate

To a solution of tert-butyl 3,3-dimethyl-4-oxopiperidine-1-carboxylate (20.0 g) in DCM (400 mL) was added Phenyltrimethylammonium tribromide (36.4 g) at 0° C. and stirred at 0° C. for 4 hours. After completion of the reaction, the mixture was diluted with water (500 mL), extracted with DCM (2*400 mL). The organic layers were washed with brine (500 mL), dried over Na₂SO₄ then concentrated under reduced pressure. The crude was purified by flash chromatography (Petroleum Ether/EtOAc 90:10) to obtain Compound 359 (9.0 g, 33%) as a brown solid.

M/Z (M[⁷⁹Br]+H-tBu)⁺: 250.2.

### Compound 360: tert-butyl 7,7-dimethyl-1,4,6,7-tetrahydro-5H-imidazo[4,5-c]pyridine-5-carboxylate

To a solution of Compound 359 (2.0 g) in EtOH (20 mL) was added formamidine acetate (6.8 g). The mixture was stirred at 100° C. for 4 hours in microwave reactor. After completion of the reaction, the mixture was concentrated. The crude was diluted with water (20 mL), extracted with EtOAc (2*20 mL). The organic layers were washed with brine (20 mL), dried over Na₂SO₄ then concentrated under reduced pressure. The crude was purified by flash chromatography (DCM/MeOH 90:10) to obtain Compound 360 (1.0 g, 61%) as a brown solid.

M/Z (M+H)⁺: 252.2.

### Compound 361: mixture of tert-butyl 3,7,7-trimethyl-3,4,6,7-tetrahydro-5H-imidazo[4,5-c]pyridine-5-carboxylate and tert-butyl 1,7,7-trimethyl-1,4,6,7-tetrahydro-5H-imidazo[4,5-c]pyridine-5-carboxylate

To a solution of Compound 360 (1.0 g) in DMF (15 mL) was added NaH (60%, 0.95 g) at 0° C. under nitrogen atmosphere. The reaction was stirred to 0° C. for 30 minutes, Iodomethane (1.13 g) was added to the mixture. The reaction was stirred to 0° C. for 2 hours. then poured into cooled water (50 mL, 0° C.) and extracted with EtOAc (2*40 mL). The organic layers were washed with brine (50 mL), dried over anhydrous sodium sulfate then concentrated. The crude was purified by flash chromatography (DCM/MeOH 90:10) to obtain Compound 361 (0.5 g, 47%) as a brown solid.

M/Z (M+H)⁺: 266.2.

### Compound 362: mixture of 3,7,7-trimethyl-4,5,6,7-tetrahydro-3H-imidazo[4,5-c]pyridine and 1,7,7-trimethyl-4,5,6,7-tetrahydro-1H-imidazo[4,5-c]pyridine

To a solution of Compound 361 (2.30 g) in DCM (20 mL) was added TFA (10 mL). The reaction was stirred at 25° C. for 2 hours then concentrated under reduced pressure. The residue was treated with NaHCO₃ (sat. aq., 25 mL) and extracted with EtOAc (2*30 mL). The organic layers were dried over Na₂SO₄ then concentrated to obtain the crude Compound 362 (0.51 g, 35%) as a brown oil.

M/Z (M+H)⁺: 166.2.

### Compound 363: 5-(4-chloro-3-fluorophenyl)-3,7,7-trimethyl-4,5,6,7-tetrahydro-3H-imidazo[4,5-c]pyridine

To a solution of Compound 362 (510 mg) and 4-Chloro-3-fluoroiodobenzene (2.37 g) in dioxane (10 mL) was added cesium carbonate (3.02 g) and RuPhos Pd G4 (263 mg). The reaction was purged with nitrogen for 3 minutes and stirred at 100° C. for 3 hours. The mixture was filtered, diluted with water (50 mL), extracted with EtOAc (2*30 mL). The organic layers were washed with brine (50 mL), dried over Na₂SO₄ then concentrated under reduced pressure. The crude was purified to obtain Compound 363 (130 mg, 14%) as a yellow solid.

M/Z (M[³⁵Cl]+H)⁺: 294.2.

### Compound 364: 2-bromo-5-(4-chloro-3-fluorophenyl)-3,7,7-trimethyl-4,5,6,7-tetrahydro-3H-imidazo[4,5-c]pyridine

To a solution of Compound 363 (130 mg) in MeCN (3 mL) was added N-Bromosuccinimide (54 mg). The reaction was stirred at 25° C. for 2 hours. The mixture was concentrated under reduced pressure and the residue was diluted with water (10 mL), extracted with EtOAc (2*10 mL). The organic layers were washed with brine (10 mL), dried over Na₂SO₄ then concentrated. The crude was purified by flash chromatography (DCM/MeOH 95:05) to obtain Compound 364 (150 mg, 91%) as a yellow solid.

M/Z (M[⁷⁹Br][³⁵Cl]+H)⁺: 372.2.

### Compound 365: methyl 5-(4-chloro-3-fluorophenyl)-3,7,7-trimethyl-4,5,6,7-tetrahydro-3H-imidazo[4,5-c]pyridine-2-carboxylate

To a solution of Compound 364 (120 mg) in MeOH (5 mL) was added XPhos (23 mg), triethylamine (66 mg) and Palladium (II) Acetate (11 mg). The reaction was stirred at 70° C. for 3 days under CO atmosphere. The mixture was filtered and concentrated and the residue was purified by flash chromatography (Petroleum Ether/EtOAc, 100:0 to 60:40) to obtain Compound 365 (30 mg, 26%) as a yellow solid.

M/Z (M[³⁵Cl]+H)⁺: 352.2.

### Compound 366: 5-(4-chloro-3-fluorophenyl)-3,7,7-trimethyl-4,5,6,7-tetrahydro-3H-imidazo[4,5-c]pyridine-2-carboxylic acid

To a solution of Compound 365 (30 mg) in THE (2 mL) was added lithium hydroxide aqueous solution (2 M in H₂O, 2 mL). Then the reaction was stirred at 70° C. for 2 hours. After completion of the reaction, the mixture was filtered and concentrated to get the crude Compound 366 (17 mg) as a yellow oil.

M/Z (M[³⁵Cl]+H)⁺: 338.2.

### Example 280: methyl 2-(6-(4-(5-(4-chloro-3-fluorophenyl)-3,7,7-trimethyl-4,5,6,7-tetrahydro-3H-imidazo[4,5-c]pyridine-2-carbonyl)-3,3-dimethylpiperazin-1-yl)pyridin-3-yl)acetate

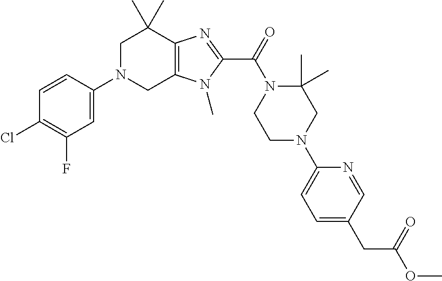 [SMILES: COC(=O)Cc1ccc(N2CCN(C(=O)c3nc4c(n3C)CN(c3ccc(Cl)c(F)c3)CC4(C)C)C(C)(C)C2)nc1]

To a solution of Compound 366 (17 mg) and Compound 111 (15.6 mg) in DMF (2 mL) was added HATU (27 mg) and N,N-Diisopropylethylamine (15.3 mg, 0.12 mmol). The reaction was stirred at 25° C. for 2 hours. After completion of the reaction, the mixture was diluted with water (10 mL), extracted with EtOAc (2*10 mL). The combined organic layers were washed with brine (10 mL), dried over Na₂SO₄ then concentrated. The crude was purified by flash chromatography (DCM/MeOH 98:2) to obtain Example 280 (15 mg, 51%) as a yellow solid.

M/Z (M[³⁵Cl]+H)⁺: 583.2.

### Example 281: 2-(6-(4-(5-(4-chloro-3-fluorophenyl)-3,7,7-trimethyl-4,5,6,7-tetrahydro-3H-imidazo[4,5-c]pyridine-2-carbonyl)-3,3-dimethylpiperazin-1-yl)pyridin-3-yl)acetic acid

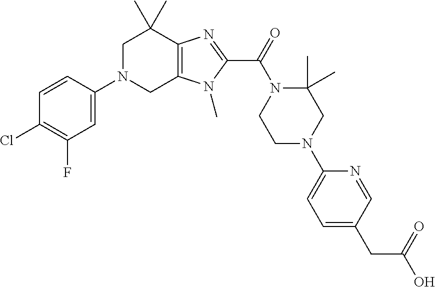 [SMILES: Cn1c(C(=O)N2CCN(c3ccc(CC(=O)O)cn3)CC2(C)C)nc2c1CN(c1ccc(Cl)c(F)c1)CC2(C)C]

To a solution of Example 280 (15 mg) in THE (1 mL) was added lithium hydroxide aqueous solution (2 M in H₂O, 1 mL). The reaction was stirred at 70° C. for 2 hours. After completion of the reaction, the mixture was filtered and concentrated. The crude was purified by preparative HPLC (H₂O/MeCN+0.05% HCOOH) to obtain Example 281 (1 mg, 7%) as a white solid.

M/Z (M[³⁵Cl]+H)⁺: 569.2.

¹H-NMR (DMSO-d₆, 400 MHz) δ: 1.22 (s, 6H, CH₃); 1.48 (s, 6H, CH₃); 3.17-3.21 (m, 2H, N—CH₂); 3.32-3.36 (m, 2H N—CH₂); 3.46-3.50 (m, 2H, N—CH₂); 3.60 (s, 3H, N—CH₃); 3.86 (s, 2H, CH₂C(O)); 4.05-4.08 (m, 2H, N—CH₂); 4.28 (s, 2H, N—CH₂); 6.52-6.57 (m, 1H, Ar); 6.87-6.93 (m, 1H, Ar), 7.03-7.11 (m, 1H, Ar), 7.35 (d, J 8.8 Hz, 1H); 7.41-7.45 (m, 1H, Ar); 7.91-7.93 (m, 1H, Ar); COOH was not observed.

### Example 282: 4-(5′-(3,4-difluorophenyl)-3,3-dimethyl-5′,6′-dihydrospiro[cyclobutane-1,7′-pyrrolo[2,3-b]pyrazine]-2′-carbonyl)-3,3-dimethylpiperazin-2-one

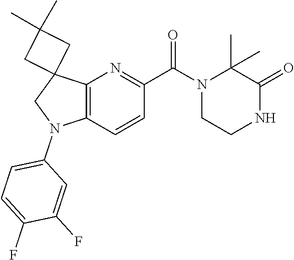 [SMILES: CC1(C)CC2(CN(c3ccc(F)c(F)c3)c3ccc(C(=O)N4CCNC(=O)C4(C)C)nc32)C1]

Example 282 was prepared according to general procedure (XIV) starting from Compound 299 (250 mg) and 3,3-dimethylpiperazin-2-one (84 mg). The crude was purified by flash chromatography (Interchim® 50 μm, CyHex 100% to CyHex/EtOAc 0:100) to obtain Example 282 (18 mg, 6%) as a beige solid.

M/Z (M+H)⁺: 456.3.

¹H-NMR (DMSO-d₆, 300 MHz) δ: 1.23 (s, 3H, CH₃); 1.29 (s, 3H, CH₃); 1.68 (s, 6H, CH₃); 2.09-2.14 (m, 2H, CH₂); 2.30-2.34 (m, 2H, CH₂); 3.37-3.43 (m, 2H, N—CH₂); 3.58-3.65 (m, 2H, N—CH₂); 4.40 (s, 2H, N—CH₂); 7.45-7.55 (m, 1H, Ar); 7.69-7.76 (m, 1H, Ar); 8.06-8.15 (m, 2H, Ar+NH); 8.23 (s, 1H, Ar).

### BIOLOGICAL EXAMPLES

### Example 283: In Vitro Human PAR-2 Functional Antagonist Activity Using Calcium Assays

Examples of the present invention were tested successively for their agonist and antagonist activities on human PAR-2 (hPAR-2) receptor expressed in HEK-293 cells in an endogenous manner. Compounds exert agonist activity if, by themselves in absence of the 2-Furoyl-LIGRLO-NH₂ peptide (selective hPAR-2 agonist), they activate PAR-2; they exert antagonist activity if they decrease the action of the 2-Furoyl-LIGRLO-NH₂ peptide on the receptor. The assay used to determine compound activity is based on calcium measurement.

Cell Culture: HEK-293 cells were maintained in Dulbecco's Modified Eagle's Medium supplemented with 10% Foetal Calf Serum, 1% Penicillin/Streptomycin at 37° C./5% CO₂. Flasks for tests (F75) are seeded with 6e+06 cells 24 h before the experiment at 37° C./5% CO₂.

Calcium assay: Receptor activity was detected by changes in intracellular calcium measured using the fluorescent Ca²⁺ sensitive dye, Fluo4AM (Molecular Probes).

The day of the assay, medium was replaced by assay buffer (HBSS 1× (Gibco 14175-053), Hepes 20 mM, MgSO₄-7H₂O 1 mM, Na₂CO₃ 3.3 mM, CaCl₂—2H₂O 2.6 mM, 0.5% bovine serum albumin (BSA), Probenecid 2.5 mM, 0.1 mg/mL Pluronic Acid) and complemented with 1 μM Fluo4AM and cells were incubated during 1:30 h at 37° C.

Cells were then detached, resuspended in assay buffer and seeded in 384 wells, transparent-flat bottom black walled plates (2e+04 cells per well). Plates were incubated at +22° C. for 20 min. Compounds stock solutions were prepared in 100% DMSO and dilutions in test buffer were carried out in polypropylene plates with a digital dispenser (D300e, Tecan). Compounds were added to the cells and intracellular Ca²⁺ measurements were collected at the same time on FLIPR Tetra® (Molecular Devices) with specific filters (Exc: 470-495 nm/Em: 515-575 nm).

Agonist and antagonist activities of compounds were consecutively evaluated on the same cell plate. Agonist activity was first measured after 10 min incubation with the compound alone. Then, cells were stimulated by an EC₅₀ of the 2-Furoyl-LIGRLO-NH₂ peptide and fluorescence was recorded for an additional 5 min. EC₅₀ of the 2-Furoyl-LIGRLO-NH₂ peptide was the concentration giving 80% of the maximal 2-Furoyl-LIGRLO-NH₂ response. Agonist activities are evaluated in comparison to basal signals evoked by assay buffer or maximal 2-Furoyl-LIGRLO-NH₂ response. None of the compounds tested were found to exhibit PAR-2 agonist activity. Antagonist activities are evaluated in comparison to basal signals evoked by assay buffer or EC₅₀ of 2-Furoyl-LIGRLO-NH₂ alone.

For IC₅₀ determination, a concentration-response test was performed using 20 or 8 concentrations (ranging over 4.5 logs) of each compound. Dose-response curves were fitted using a sigmoidal dose-response 4 parameters (variable slope) analysis in XLfit Excel addon (IDBS) and IC₅₀ of antagonist activity was calculated. Concentration-response experiments were performed in duplicate, in two independent experiments. IC₅₀ values are categorized as following: A: IC₅₀<500 nM; B: 500 nM≤IC₅₀≤10 μM; C: IC₅₀>10 μM
(A)—IC₅₀<500 nM: Examples 3, 4, 8, 9, 12, 14, 15, 17, 18, 19, 20, 21, 27, 31, 39, 41, 43, 45, 53, 55, 56, 57, 58, 60, 61, 62, 65, 67, 69, 71, 73, 74, 79, 80, 81, 82, 83, 85, 89, 90, 96, 100, 101, 103, 105, 113, 115, 117, 119, 126, 128, 134, 140, 142, 144, 146, 148, 151, 154, 156, 158, 160, 164, 171, 173, 175, 177, 181, 185, 187, 189, 191, 193, 199, 201, 203, 205, 207, 209, 215, 217, 219, 221, 223, 225, 227 229, 231, 233, 235, 237, 239, 241, 243, 245, 247, 249, 251, 259, 260, 275, 277, 279, 282.
(B)—IC₅₀ between 500 nM and 10 μM: Examples 1, 2, 6, 10, 11, 22, 23, 24, 26, 28, 29, 33, 35, 37, 47, 48, 50, 51, 54, 63, 75, 76, 77, 92, 94, 98, 107, 111, 121, 125, 130, 135, 137, 138, 162, 165, 167, 169, 195, 211, 213, 253, 257, 258, 261, 270, 273.
(C)—IC₅₀>10 μM: Examples 52, 87, 109, 120, 123, 133, 152, 179, 183, 197, 255, 263, 265, 266, 268.

It has thus been demonstrated that the compounds of formula (I), including in particular the above-described examples, are potent antagonists of PAR-2.

1. A compound of the following formula (I) 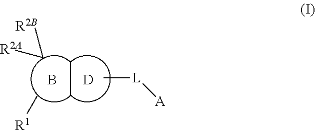
wherein:
ring B is a non-aromatic C₄₈ carbocyclic ring or a non-aromatic 4- to 8-membered heterocyclic ring, which is fused to ring D,
wherein said carbocyclic ring or said heterocyclic ring is: (i) substituted with a group R¹; (ii) substituted with the groups R^{2A} and R^{2B} which are attached to the same ring carbon atom of said carbocyclic ring or said heterocyclic ring; and (iii) optionally substituted with one or more groups R^{Y};
ring D is a 5- or 6-membered heteroaromatic ring, which is fused to ring B, wherein said heteroaromatic ring comprises at least one nitrogen ring atom, wherein said heteroaromatic ring is substituted with a group -L-A, and wherein said heteroaromatic ring is optionally substituted with one or more groups R^{X};
R¹ is selected from C₁₋₅ alkyl, C₂₋₅ alkenyl, C₂₋₅ alkynyl, —(C₀₋₅ alkylene)-carbocyclyl, and —(C₀₋₅ alkylene)-heterocyclyl,
wherein said alkyl, said alkenyl, said alkynyl, the alkylene group in said —(C₀₋₅ alkylene)-carbocyclyl, and the alkylene group in said —(C₀₋₅ alkylene)-heterocyclyl are each optionally substituted with one or more groups R¹², wherein one or more —CH₂— units comprised in said alkyl, said alkenyl, said alkynyl, in the alkylene group in said —(C₀₋₅ alkylene)-carbocyclyl, or in the alkylene group in said —(C₀₋₅ alkylene)-heterocyclyl are each optionally replaced by a group independently selected from —C(R^{L1})(R^{L1})—, —O—, —S—, —SO—, —SO₂—, —CO—, and —N(R^{L1})—, wherein each R^{L1} is independently hydrogen or C₁₋₅ alkyl, wherein two groups R^{L1} which are attached to the same carbon atom may also be mutually joined to form, together with the carbon atom that they are attached to, a cycloalkyl or a heterocycloalkyl,
and wherein the carbocyclyl group in said —(C₀₋₅ alkylene)-carbocyclyl and the heterocyclyl group in said —(CO₅ alkylene)-heterocyclyl are each optionally substituted with one or more groups R¹¹;
each R¹¹ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc};
each R¹² is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), carbocyclyl, heterocyclyl, and -L^{Z}-R^{Z}, wherein said carbocyclyl and said heterocyclyl are each optionally substituted with one or more groups R^{Cyc};
R^{2A} and R^{2B} are mutually joined to form, together with the carbon atom that they are attached to, a cycloalkyl, cycloalkenyl, heterocycloalkyl, or heterocycloalkenyl, wherein said cycloalkyl, said cycloalkenyl, said heterocycloalkyl or said heterocycloalkenyl is optionally substituted with one or more groups R²¹;
or R^{2A} and R^{2B} are each independently selected from C₁₋₅ alkyl, C₂₋₅ alkenyl, C₂₋₅ alkynyl, —(C₀₋₅ alkylene)-carbocyclyl, and —(C₀₋₅ alkylene)-heterocyclyl, wherein said alkyl, said alkenyl, said alkynyl, the alkylene group in said —(C₀₋₅ alkylene)-carbocyclyl, and the alkylene group in said —(C₀₋₅ alkylene)-heterocyclyl are each optionally substituted with one or more groups R²², wherein one or more —CH₂— units comprised in said alkyl, said alkenyl, said alkynyl, in the alkylene group in said —(C₀₋₅ alkylene)-carbocyclyl, or in the alkylene group in said —(C₀₋₅ alkylene)-heterocyclyl are each optionally replaced by a group independently selected from —O—, —NH—, —N(C₁₋₅ alkyl)-, —CO—, —S—, —SO—, and —SO₂—, and further wherein the carbocyclyl group in said —(C₀₋₅ alkylene)-carbocyclyl and the heterocyclyl group in said —(C₀₋₅ alkylene)-heterocyclyl are each optionally substituted with one or more groups R^{Cyc},
wherein R^{2A} and a group R^{Y} may also be mutually joined to form, together with the ring atoms that they are attached to, a carbocyclyl or heterocyclyl, wherein said carbocyclyl or said heterocyclyl is optionally substituted with one or more groups R^{Cyc};
each R²¹ is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc};
each R²² is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), carbocyclyl, heterocyclyl, and -L^{Z}-R^{Z}, wherein said carbocyclyl and said heterocyclyl are each optionally substituted with one or more groups R^{Cyc};
each R^{X} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc};
each R^{Y} is independently selected from C₁₋₅ alkyl, C₂₋₅ alkenyl, C₂₋₅ alkynyl, —(C₀₋₃ alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc};
wherein any two groups R^{Y} which are attached to the same ring carbon atom (i) may also be mutually joined to form, together with the carbon atom that they are attached to, a cycloalkyl or a heterocycloalkyl, wherein said cycloalkyl or said heterocycloalkyl is optionally substituted with one or more groups R^{Cyc}, or (ii) may mutually form a group ═O;
L is selected from —CO—, —SO— and —SO₂—;
group A is —N(—R^{N})—R^{N} or heterocyclyl, wherein said heterocyclyl is attached via a ring nitrogen atom to group L, and wherein said heterocyclyl is optionally substituted with one or more groups R^{A};
each R^{N} is independently selected from hydrogen, C₁₋₈ alkyl, C₂₋₈ alkenyl, C₂₋₈ alkynyl, —(C₀₋₈ alkylene)-OH, —(C₀₋₈ alkylene)-O(C₁₋₅ alkyl), —(C₀₋₈ alkylene)-SH, —(C₀₋₈ alkylene)-S(C₁₋₅ alkyl), —(C₁₋₈ alkylene)-NH₂, —(C₁₋₈ alkylene)-NH(C₁₋₅ alkyl), —(C₁₋₈ alkylene)-N(C₁₋₅ alkyl)(C₁₋₅ alkyl), —(C₁₋₈ alkylene)-halogen, —(C₁₋₈ alkylene)-C₁₋₅ haloalkyl, —(C₀₋₈ alkylene)-O—(C₁₋₈ haloalkyl), —(C₀₋₈ alkylene)-CN, —(C₀₋₈ alkylene)-CHO, —(C₀₋₈ alkylene)-CO—(C₁₋₅ alkyl), —(C₀₋₈ alkylene)-COOH, —(C₀₋₈ alkylene)-CO—O—(C₁₋₅ alkyl), —(C₀₋₈ alkylene)-O—CO—(C₁₋₅ alkyl), —(C₀₋₈ alkylene)-CO—NH₂, —(C₀₋₈ alkylene)-CO—NH(C₁₋₅ alkyl), —(C₀₋₈ alkylene)-CO—N(C₁₋₅ alkyl)(C₁₋₅ alkyl), —(C₁₋₈ alkylene)-NH—CO—(C₁₋₅ alkyl), —(C₁₋₈ alkylene)-N(C₁₋₅ alkyl)-CO—(C₁₋₅ alkyl), —(C₁₋₈ alkylene)-NH—COO(C₁₋₅ alkyl), —(C₁₋₈ alkylene)-N(C₁₋₅ alkyl)-COO(C₁₋₅ alkyl), —(C₀₋₈ alkylene)-O—CO—NH(C₁₋₅ alkyl), —(C₀₋₈ alkylene)-O—CO—N(C₁₋₅ alkyl)(C₁₋₅ alkyl), —(C₀₋₈ alkylene)-SO₂—NH₂, —(C₀₋₈ alkylene)-SO₂—NH(C₁₋₅ alkyl), —(C₀₋₈ alkylene)-SO₂—N(C₁₋₅ alkyl)(C₁₋₅ alkyl), —(C₁₋₈ alkylene)-NH—SO₂—(C₁₋₅ alkyl), —(C₁₋₈ alkylene)-N(C₁₋₅ alkyl)-SO₂—(C₁₋₅ alkyl), —(C₀₋₈ alkylene)-SO—(C₁₋₅ alkyl), —(C₀₋₈ alkylene)-SO₂—(C₁₋₅ alkyl), —(C₀₋₈ alkylene)-carbocyclyl, and —(C₀₋₈ alkylene)-heterocyclyl, wherein one or more —CH₂— units comprised in said C₁₋₈ alkyl, said C₂₋₈ alkenyl, said C₂₋₈ alkynyl, and in any of the aforementioned C₀₋₈ alkylene and C₁₋₈ alkylene groups are each optionally replaced by a group independently selected from —O—, —NH—, —N(C₁₋₅ alkyl)-, —CO—, —S—, —SO—, and —SO₂—, wherein the carbocyclyl group in said —(C₀₋₈ alkylene)-carbocyclyl and the heterocyclyl group in said —(C₀₋₈ alkylene)-heterocyclyl are each optionally substituted with one or more groups R^{Cyc}, and wherein at least one group R^{N} is not hydrogen;
each R^{A} is independently selected from C₁₋₅ alkyl, C₂₋₅ alkenyl, C₂₋₅ alkynyl, —(CO-3 alkylene)-OH, —(C₀₋₃ alkylene)-O(C₁₋₅ alkyl), —(C₀₋₃ alkylene)-O(C₁₋₅ alkylene)-OH, —(C₀₋₃ alkylene)-O(C₁₋₅ alkylene)-O(C₁₋₅ alkyl), —(C₀₋₃ alkylene)-SH, —(C₀₋₃ alkylene)-S(C₁₋₅ alkyl), —(C₀₋₃ alkylene)-S(C₁₋₅ alkylene)-SH, —(C₀₋₃ alkylene)-S(C₁₋₅ alkylene)-S(C₁₋₅ alkyl), —(C₀₋₃ alkylene)-NH₂, —(C₀₋₃ alkylene)-NH(C₁₋₅ alkyl), —(C₀₋₃ alkylene)-N(C₁₋₅ alkyl)(C₁₋₅ alkyl), —(C₀₋₃ alkylene)-NH—OH, —(C₀₋₃ alkylene)-N(C₁₋₅ alkyl)-OH, —(C₀₋₃ alkylene)-NH—O(C₁₋₅ alkyl), —(C₀₋₃ alkylene)-N(C₁₋₅ alkyl)-O(C₁₋₅ alkyl), —(C₀₋₃ alkylene)-halogen, —(C₀₋₃ alkylene)-(C₁₋₅ haloalkyl), —(C₀₋₃ alkylene)-O—(C₁₋₅ haloalkyl), —(C₀₋₃ alkylene)-CN, —(C₀₋₃ alkylene)-CHO, —(C₀₋₃ alkylene)-CO—(C₁₋₅ alkyl), —(C₀₋₃ alkylene)-COOH, —(C₀₋₃ alkylene)-CO—O—(C₁₋₅ alkyl), —(C₀₋₃ alkylene)-O—CO—(C₁₋₅ alkyl), —(C₀₋₃ alkylene)-CO—NH₂, —(C₀₋₃ alkylene)-CO—NH(C₁₋₅ alkyl), —(C₀₋₃ alkylene)-CO—N(C₁₋₅ alkyl)(C₁₋₅ alkyl), —(C₀₋₃ alkylene)-NH—CO—(C₁₋₅ alkyl), —(C₀₋₃ alkylene)-N(C₁₋₅ alkyl)-CO—(C₁₋₅ alkyl), —(C₀₋₃ alkylene)-NH—COO(C₁₋₅ alkyl), —(C₀₋₃ alkylene)-N(C₁₋₅ alkyl)-COO(C₁₋₅ alkyl), —(C₀₋₃ alkylene)-O—CO—NH(C₁₋₅ alkyl), —(C₀₋₃ alkylene)-O—CO—N(C₁₋₅ alkyl)(C₁₋₅ alkyl), —(C₀₋₃ alkylene)-SO₂—NH₂, —(C₀₋₃ alkylene)-SO₂—NH(C₁₋₅ alkyl), —(C₀₋₃ alkylene)-SO₂—N(C₁₋₅ alkyl)(C₁₋₅ alkyl), —(C₀₋₃ alkylene)-NH—SO₂—(C₁₋₅ alkyl), —(C₀₋₃ alkylene)-N(C₁₋₅ alkyl)-SO₂—(C₁₋₅ alkyl), —(C₀₋₃ alkylene)-SO—(C₁₋₅ alkyl), —(C₀₋₃ alkylene)-SO₂—(C₁₋₅ alkyl), —(C₀₋₃ alkylene)-carbocyclyl, —(C₀₋₃ alkylene)-heterocyclyl, and -L^{Z}-R^{Z}, wherein the carbocyclyl group in said —(C₀₋₃ alkylene)-carbocyclyl and the heterocyclyl group in said —(C₀₋₃ alkylene)-heterocyclyl are each optionally substituted with one or more groups R^{Cyc}; and further wherein any two groups R^{A}, which are attached to the same carbon atom of group A, may also be mutually joined to form, together with the carbon atom that they are attached to, a cycloalkyl or a heterocycloalkyl, wherein said cycloalkyl or said heterocycloalkyl is optionally substituted with one or more groups R^{Cyc};
each R^{Cyc} is independently selected from C₁₋₅ alkyl, C₂₋₅ alkenyl, C₂₋₅ alkynyl, —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), —P(═O)(—OH)(—OH), —P(═O)(—OH)(—O—C₁₋₅ alkyl), —P(═O)(—O—C₁₋₅ alkyl)(—O—C₁₋₅ alkyl), —(C₀₋₃ alkylene)-cycloalkyl, —(C₀₋₃ alkylene)-heterocycloalkyl, and -L^{Z}-R^{Z};
each L^{Z} is independently selected from a covalent bond, C₁₇ alkylene, C₂₇ alkenylene, and C₂₇ alkynylene, wherein said alkylene, said alkenylene and said alkynylene are each optionally substituted with one or more groups independently selected from halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), and —N(C₁₋₅ alkyl)(C₁₋₅ alkyl), and further wherein one or more —CH₂— units comprised in said alkylene, said alkenylene or said alkynylene are each optionally replaced by a group independently selected from —O—, —NH—, —N(C₁₋₅ alkyl)-, —CO—, —S—, —SO—, and —SO₂—; and
each R^{Z} is independently selected from —OH, —O(C₁₋₅ alkyl), —O(C₁₋₅ alkylene)-OH, —O(C₁₋₅ alkylene)-O(C₁₋₅ alkyl), —SH, —S(C₁₋₅ alkyl), —S(C₁₋₅ alkylene)-SH, —S(C₁₋₅ alkylene)-S(C₁₋₅ alkyl), —NH₂, —NH(C₁₋₅ alkyl), —N(C₁₋₅ alkyl)(C₁₋₅ alkyl), —NH—OH, —N(C₁₋₅ alkyl)-OH, —NH—O(C₁₋₅ alkyl), —N(C₁₋₅ alkyl)-O(C₁₋₅ alkyl), halogen, C₁₋₅ haloalkyl, —O—(C₁₋₅ haloalkyl), —CN, —CHO, —CO(C₁₋₅ alkyl), —COOH, —COO(C₁₋₅ alkyl), —O—CO(C₁₋₅ alkyl), —CO—NH₂, —CO—NH(C₁₋₅ alkyl), —CO—N(C₁₋₅ alkyl)(C₁₋₅ alkyl), —NH—CO(C₁₋₅ alkyl), —N(C₁₋₅ alkyl)-CO(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO₂—(C₁₋₅ alkyl), —SO—(C₁₋₅ alkyl), aryl, heteroaryl, cycloalkyl, and heterocycloalkyl, wherein said aryl, said heteroaryl, said cycloalkyl, and said heterocycloalkyl are each optionally substituted with one or more groups independently selected from C₁₋₅ alkyl, C₂₋₅ alkenyl, C₂₋₅ alkynyl, halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), —SO₂—(C₁₋₅ alkyl), carbocyclyl, and heterocyclyl, wherein said carbocyclyl and said heterocyclyl are each optionally substituted with one or more groups independently selected from C₁₋₅ alkyl, C₂₋₅ alkenyl, C₂₋₅ alkynyl, halogen, C₁₋₅ haloalkyl, —O—(C₁₋₅ haloalkyl), —CN, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), —CHO, —CO—(C₁₋₅ alkyl), —COOH, —CO—O—(C₁₋₅ alkyl), —O—CO—(C₁₋₅ alkyl), —CO—NH₂, —CO—NH(C₁₋₅ alkyl), —CO—N(C₁₋₅ alkyl)(C₁₋₅ alkyl), —NH—CO—(C₁₋₅ alkyl), —N(C₁₋₅ alkyl)-CO—(C₁₋₅ alkyl), —NH—COO(C₁₋₅ alkyl), —N(C₁₋₅ alkyl)-COO(C₁₋₅ alkyl), —O—CO—NH(C₁₋₅ alkyl), —O—CO—N(C₁₋₅ alkyl)(C₁₋₅ alkyl), —SO₂—NH₂, —SO₂—NH(C₁₋₅ alkyl), —SO₂—N(C₁₋₅ alkyl)(C₁₋₅ alkyl), —NH—SO₂—(C₁₋₅ alkyl), —N(C₁₋₅ alkyl)-SO₂—(C₁₋₅ alkyl), —SO—(C₁₋₅ alkyl), and —SO₂—(C₁₋₅ alkyl);
with the proviso that:
if ring B is a pyrrolidinyl ring, ring D is a pyridinyl ring, R^{2A} and R^{2B} are mutually joined to form, together with the carbon atom that they are attached to, a cyclopropyl, L is —CO—, and group A is morpholin-4-yl, then R¹ is not 5-R¹¹-pyrimidin-2-yl or acetyl;
if ring B and ring D together are a 2-oxo-2,3-dihydro-1H-pyrrolo[2,3-b]pyridinyl ring, a 2-oxo-2,3-dihydro-1H-pyrrolo[2,3-c]pyridinyl ring or a 6-oxo-6,7-dihydro-5H-pyrrolo[2,3-c]pyridazinyl ring, R^{2A} and R^{2B} are each methyl, R¹ is phenyl which is optionally substituted with one or more groups R¹¹, L is —CO—, and group A is —NH—R^{N}, then R^{N} is not a heterocycloalkyl which comprises one oxidized sulfur ring atom, in which all other ring atoms are carbon atoms, and which is substituted with a methyl group;
if ring B and ring D together are a 3-R^{X}-4-oxo-4,5,6,7-tetrahydropyrazolo[1,5-a]pyrazinyl ring, R^{X} is —OH, one of R^{2A} and R^{2B} is methyl, the other one of R^{2A} and R^{2B} is —CON(—CH₃)₂, R¹ is methyl, L is —CO—, and group A is —NH—R^{N}, then R^{N} is not 4-fluorobenzyl; and
if ring B and ring D together are a 2-oxo-2,3-dihydro-1H-pyrrolo[3,2-b]pyridine ring, a 2-oxo-2,3-dihydro-1H-pyrrolo[2,3-b]pyridine ring or a 2-oxo-2,3-dihydro-1H-pyrrolo[3,2-c]pyridine, L is —CO—, group A is —NH—R^{N}, and R¹ is —CH₂-phenyl or —CH₂-pyridinyl, wherein the phenyl in said —CH₂-phenyl and the pyridinyl in said —CH₂-pyridinyl are each optionally substituted with one or more groups R¹¹, then R^{2A} and R^{2B} are not methyl;
or a pharmaceutically acceptable salt or solvate thereof.

2. The compound of claim 1, wherein ring B is a non-aromatic 4- to 8-membered heterocyclic ring which is optionally substituted with one or more groups R^{Y}.

3. The compound of claim 1 or 2, wherein ring B is a pyrrolidinyl ring or a piperidinyl ring, wherein said pyrrolidinyl ring or said piperidinyl ring is optionally substituted with one or more groups R^{Y}, and wherein the group R¹ is attached to the nitrogen ring atom of said pyrrolidinyl ring or said piperidinyl ring.

4. The compound of any one of claims 1 to 3, wherein ring B is a group of the following formula: 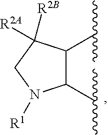
which is optionally substituted with one or two groups R^{Y}.

5. The compound of any one of claims 1 to 4, wherein ring D is a 6-membered heteroaromatic ring which comprises one or two nitrogen ring atoms while all remaining ring atoms are carbon atoms, and wherein said heteroaromatic ring is optionally substituted with one or more groups R^{X}.

6. The compound of any one of claims 1 to 5, wherein ring D is a pyridinyl ring or a pyrazinyl ring, wherein said pyridinyl ring or said pyrazinyl ring is optionally substituted with one or more groups R^{X}.

7. The compound of any one of claims 1 to 6, wherein ring D is a pyrazinyl ring which is optionally substituted with one or more groups R^{X}, wherein said pyrazinyl ring is fused via its ring carbon atoms 2 and 3 to ring B.

8. The compound of any one of claims 1 to 7, wherein the compound of formula (I) has the following structure:

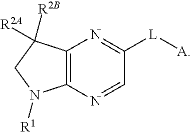

9. The compound of any one of claims 1 to 8, wherein R¹ is -L¹-carbocyclyl or -L¹-heterocyclyl, wherein the carbocyclyl in said -L¹-carbocyclyl or the heterocyclyl in said -L¹-heterocyclyl is optionally substituted with one or more groups R¹¹, wherein each L¹ is independently selected from a bond, —C(R^{L1})(R^{L1})—, —S—, —SO—, —SO₂—, —CO—, and —N(R^{L1})—, wherein each R^{L1} is independently hydrogen or C₁₋₅ alkyl, and further wherein two groups R^{L1} which are attached to the same carbon atom may also be mutually joined to form, together with the carbon atom that they are attached to, a cycloalkyl or a heterocycloalkyl.

10. The compound of any one of claims 1 to 9, wherein R¹ is phenyl which is optionally substituted with one or more groups R¹¹.

11. The compound of any one of claims 1 to 10, wherein R¹ is 3-R¹¹-4-R¹¹-phenyl or 3-R¹¹-4-R¹¹-5-R¹¹-phenyl, wherein each R¹¹ is independently selected from halogen, C₁₋₅ haloalkyl, and C₁₋₅ alkyl;
preferably wherein R¹ is selected from 4-chloro-3-fluoro-phenyl, 3,4-dichloro-phenyl, 3,4-difluoro-phenyl, 3-chloro-4-fluoro-phenyl, 3-fluoro-4-trifluoromethyl-phenyl, 3-chloro-4-trifluoromethyl-phenyl, 3-fluoro-4-methyl-phenyl, 3-chloro-4-methyl-phenyl, 3,4,5-trifluoro-phenyl, and 4-chloro-3,5-difluoro-phenyl.

12. The compound of any one of claims 1 to 11, wherein R^{2A} and R^{2B} are mutually joined to form, together with the carbon atom that they are attached to, a cycloalkyl or heterocycloalkyl, wherein said cycloalkyl or said heterocycloalkyl is optionally substituted with one or more groups R²¹.

13. The compound of any one of claims 1 to 12, wherein R^{2A} and R^{2B} are mutually joined to form, together with the carbon atom that they are attached to, a cyclopentyl or a tetrahydrofuranyl, wherein said cyclopentyl or said tetrahydrofuranyl is optionally substituted with one or more groups R²¹.

14. The compound of any one of claims 1 to 11, wherein R^{2A} and R^{2B} are each independently selected from C₁₋₅ alkyl, —(C₀₋₅ alkylene)-cycloalkyl, —(C₀₋₅ alkylene)-aryl, —(C₀₋₅ alkylene)-heterocycloalkyl, and —(C₀₋₅ alkylene)-heteroaryl, wherein said alkyl or the alkylene group in any of said —(C₀₋₅ alkylene)-cycloalkyl, said —(C₀₋₅ alkylene)-aryl, said —(C₀₋₅ alkylene)-heterocycloalkyl, or said —(C₀₋₅ alkylene)-heteroaryl is optionally substituted with one or more groups independently selected from —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN, and further wherein the cycloalkyl group in said —(C₀₋₅ alkylene)-cycloalkyl, the aryl group in said —(C₀₋₅ alkylene)-aryl, the heterocycloalkyl group in said —(C₀₋₅ alkylene)-heterocycloalkyl, and the heteroaryl group in said —(C₀₋₅ alkylene)-heteroaryl are each optionally substituted with one or more groups independently selected from C₁₋₅ alkyl, C₂₋₅ alkenyl, C₂₋₅ alkynyl, —OH, —O(C₁₋₅ alkyl), —SH, —S(C₁₋₅ alkyl), —NH₂, —NH(C₁₋₅ alkyl), —N(C₁₋₅ alkyl)(C₁₋₅ alkyl), halogen, C₁₋₅ haloalkyl, —O—(C₁₋₅ haloalkyl), and —CN.

15. The compound of any one of claims 1 to 11 or 14, wherein R^{2A} and R^{2B} are each independently C₁₋₅ alkyl; preferably wherein R^{2A} and R^{2B} are each methyl.

16. The compound of any one of claims 1 to 15, wherein L is —CO—.

17. The compound of any one of claims 1 to 16, wherein group A is heterocycloalkyl which is attached via a ring nitrogen atom to group L, wherein said heterocycloalkyl is optionally substituted with one or more groups R^{A}.

18. The compound of any one of claims 1 to 17, wherein group A is 2,2-dimethyl-piperazin-1-yl, wherein the piperazinyl group in said 2,2-dimethyl-piperazin-1-yl is optionally substituted with one or more groups R^{A};
preferably wherein group A is 2,2-dimethyl-4-(5-carboxy-4,6-dimethyl-pyridin-2-yl)piperazin-1-yl.

19. The compound of claim 1, wherein said compound is selected from:
1′-(4-chloro-3-fluorophenyl)-5′-(2,2-dimethyl-3-oxopiperazine-1-carbonyl)-2,3,5,6-tetrahydrospiro[pyran-4,3′-pyrrolo[3,2-b]pyridin]-2′(1′H)-one;
1′-(4-chloro-3-fluorophenyl)-5′-(2,2-dimethyl-3-oxopiperazine-1-carbonyl)spiro[cyclopentane-1,3′-pyrrolo[3,2-b]pyridin]-2′(1′H)-one;
4-(1′-(4-chloro-3-fluorophenyl)-1′,2′-dihydrospiro[cyclopentane-1,3′-pyrrolo[3,2-b]pyridine]-5′-carbonyl)-3,3-dimethylpiperazin-2-one;
8-(1′-(4-chloro-3-fluorophenyl)-1′,2′-dihydrospiro[cyclopentane-1,3′-pyrrolo[3,2-b]pyridine]-5′-carbonyl)-1,3,8-triazaspiro[4.5]decane-2,4-dione;
methyl 1-(1′-(4-chloro-3-fluorophenyl)-1′,2′-dihydrospiro[cyclopentane-1,3′-pyrrolo[3,2-b]pyridine]-5′-carbonyl)-3,3-dimethylpiperidine-4-carboxylate;
1-(1′-(4-chloro-3-fluorophenyl)-1′,2′-dihydrospiro[cyclopentane-1,3′-pyrrolo[3,2-b]pyridine]-5′-carbonyl)-3,3-dimethylpiperidine-4-carboxylic acid;
methyl 6-(4-(1′-(4-chloro-3-fluorophenyl)-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
6-(4-(1′-(4-chloro-3-fluorophenyl)-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
4-(1′-(3-chlorophenyl)-1′,2′-dihydrospiro[cyclopentane-1,3′-pyrrolo[3,2-b]pyridine]-5′-carbonyl)-3,3-dimethylpiperazin-2-one;
4-(1′-(2-chlorophenyl)-1′,2′-dihydrospiro[cyclopentane-1,3′-pyrrolo[3,2-b]pyridine]-5′-carbonyl)-3,3-dimethylpiperazin-2-one;
4-(3,3-dimethyl-1-(thiophen-3-yl)-2,3-dihydro-1H-pyrrolo[3,2-b]pyridine-5-carbonyl)-3,3-dimethylpiperazin-2-one;
4-(1-(3,4-difluorophenyl)-3,3-dimethyl-2,3-dihydro-1H-pyrrolo[3,2-b]pyridine-5-carbonyl)-3,3-dimethylpiperazin-2-one;
methyl 6-(4-(1-(3,4-difluorophenyl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(3,4-difluorophenyl)-3,3-dimethyl-2,3-dihydro-1H-pyrrolo[3,2-b]pyridine-5-carbonyl)-3,3-dimethylpiperazin-1-yl)-2,4-dimethylnicotinic acid;
4-(1-(4-fluorophenyl)-3,3-dimethyl-2,3-dihydro-1H-pyrrolo[3,2-b]pyridine-5-carbonyl)-3,3-dimethylpiperazin-2-one;
methyl 6-(4-(1-(3-fluorophenyl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(3-fluorophenyl)-3,3-dimethyl-2,3-dihydro-1H-pyrrolo[3,2-b]pyridine-5-carbonyl)-3,3-dimethylpiperazin-1-yl)-2,4-dimethylnicotinic acid;
4-(1-(4-chloro-3-fluorophenyl)-3,3-dimethyl-2,3-dihydro-1H-pyrrolo[3,2-b]pyridine-5-carbonyl)-3,3-dimethylpiperazin-2-one;
4-(1-(4-chloro-3-fluorophenyl)-3,3-dimethyl-2,3-dihydro-1H-pyrrolo[3,2-b]pyridine-5-carbonyl)-1-ethyl-3,3-dimethylpiperazin-2-one;
(1-(4-chloro-3-fluorophenyl)-3,3-dimethyl-2,3-dihydro-1H-pyrrolo[3,2-b]pyridin-5-yl)(3,3-dimethylmorpholino)methanone;
tert-butyl 4-(1-(4-chloro-3-fluorophenyl)-3,3-dimethyl-2,3-dihydro-1H-pyrrolo[3,2-b]pyridine-5-carbonyl)-3,3-dimethylpiperazine-1-carboxylate;
(1-(4-chloro-3-fluorophenyl)-3,3-dimethyl-2,3-dihydro-1H-pyrrolo[3,2-b]pyridin-5-yl)(2,2-dimethylpiperazin-1-yl)methanone;
(1-(4-chloro-3-fluorophenyl)-3,3-dimethyl-2,3-dihydro-1H-pyrrolo[3,2-b]pyridin-5-yl)(4-ethyl-2,2-dimethylpiperazin-1-yl)methanone;
1-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)ethan-1-one;
ethyl 4-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-oxobutanoate;
4-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-oxobutanoic acid;
ethyl 5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-5-oxopentanoate;
5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-5-oxopentanoic acid;
8-(1-(4-chloro-3-fluorophenyl)-3,3-dimethyl-2,3-dihydro-1H-pyrrolo[3,2-b]pyridine-5-carbonyl)-1-methyl-1,3,8-triazaspiro[4.5]decan-4-one;
2-(3-(1-(4-chloro-3-fluorophenyl)-3,3-dimethyl-2,3-dihydro-1H-pyrrolo[3,2-b]pyridine-5-carbonyl)-3-azabicyclo[3.1.0]hexan-6-yl)acetic acid;
methyl 2-(3-(1-(4-chloro-3-fluorophenyl)-3,3-dimethyl-2,3-dihydro-1H-pyrrolo[3,2-b]pyridine-5-carbonyl)-3-azabicyclo[3.1.0]hexan-6-yl)acetate;
methyl 2-((1R,5S,6S)-3-(1-(4-chloro-3-fluorophenyl)-3,3-dimethyl-2,3-dihydro-1H-pyrrolo[3,2-b]pyridine-5-carbonyl)-3-azabicyclo[3.1.0]hexan-6-yl)acetate;
2-((1R,5S,6S)-3-(1-(4-chloro-3-fluorophenyl)-3,3-dimethyl-2,3-dihydro-1H-pyrrolo[3,2-b]pyridine-5-carbonyl)-3-azabicyclo[3.1.0]hexan-6-yl)acetic acid;
ethyl 1-(1-(4-chloro-3-fluorophenyl)-3,3-dimethyl-2,3-dihydro-1H-pyrrolo[3,2-b]pyridine-5-carbonyl)piperidine-4-carboxylate;
1-(1-(4-chloro-3-fluorophenyl)-3,3-dimethyl-2,3-dihydro-1H-pyrrolo[3,2-b]pyridine-5-carbonyl)piperidine-4-carboxylic acid;
methyl 2-(1-(1-(4-chloro-3-fluorophenyl)-3,3-dimethyl-2,3-dihydro-1H-pyrrolo[3,2-b]pyridine-5-carbonyl)piperidin-4-yl)acetate;
2-(1-(1-(4-chloro-3-fluorophenyl)-3,3-dimethyl-2,3-dihydro-1H-pyrrolo[3,2-b]pyridine-5-carbonyl)piperidin-4-yl)acetic acid;
2-(1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetic acid;
methyl 2-(1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetate;
methyl 2-((3R,4S)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetate;
2-((3R,4S)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetic acid;
ethyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylpyrimidine-5-carboxylate;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylpyrimidine-5-carboxylic acid;
ethyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyrimidine-5-carboxylate;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-pyrimidine-5-carboxylic acid;
ethyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)thiazole-4-carboxylate;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)thiazole-4-carboxylic acid;
methyl 6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-2,4-dimethylnicotinic acid;
3-(1-(4-chloro-3-fluorophenyl)-N,3,3-trimethyl-2,3-dihydro-1H-pyrrolo[3,2-b]pyridine-5-carboxamido)butanoic acid;
methyl 3-(1-(4-chloro-3-fluorophenyl)-N,3,3-trimethyl-2,3-dihydro-1H-pyrrolo[3,2-b]pyridine-5-carboxamido)butanoate;
methyl (R)-3-(1-(4-chloro-3-fluorophenyl)-N,3,3-trimethyl-2,3-dihydro-1H-pyrrolo[3,2-b]pyridine-5-carboxamido)butanoate;
(R)-3-(1-(4-chloro-3-fluorophenyl)-N,3,3-trimethyl-2,3-dihydro-1H-pyrrolo[3,2-b]pyridine-5-carboxamido)butanoic acid;
N-(1-carbamoylcyclopropyl)-1-(4-chloro-3-fluorophenyl)-N,3,3-trimethyl-2,3-dihydro-1H-pyrrolo[3,2-b]pyridine-5-carboxamide;
methyl 6-((2-(1-(4-chloro-3-fluorophenyl)-N,3,3-trimethyl-2,3-dihydro-1H-pyrrolo[3,2-b]pyridine-5-carboxamido)ethyl)(methyl)amino)-2,4-dimethylnicotinate;
6-((2-(1-(4-chloro-3-fluorophenyl)-N,3,3-trimethyl-2,3-dihydro-1H-pyrrolo[3,2-b]pyridine-5-carboxamido)ethyl)(methyl)amino)-2,4-dimethylnicotinic acid;
1-(4-chloro-3-fluorophenyl)-N-(1-hydroxy-2-methylpropan-2-yl)-3,3-dimethyl-2,3-dihydro-1H-pyrrolo[3,2-b]pyridine-5-carboxamide;
1-(4-chloro-3-fluorophenyl)-N-(2-hydroxy-2-methylpropyl)-3,3-dimethyl-2,3-dihydro-1H-pyrrolo[3,2-b]pyridine-5-carboxamide;
4-(1-(4-chloro-3-fluorophenyl)-3-ethyl-3-methyl-2,3-dihydro-1H-pyrrolo[3,2-b]pyridine-5-carbonyl)-3,3-dimethylpiperazin-2-one;
N-(1-carbamoylcyclopropyl)-1-(4-chloro-3-fluorophenyl)-3-ethyl-3-methyl-2,3-dihydro-1H-pyrrolo[3,2-b]pyridine-5-carboxamide;
4-(1′-(4-chloro-3-fluorophenyl)-1′,2,2′,3,5,6-hexahydrospiro[pyran-4,3′-pyrrolo[3,2-b]pyridine]-5′-carbonyl)-3,3-dimethylpiperazin-2-one;
6-(4-(1′-(4-chloro-3-fluorophenyl)-1′,2,2′,3,5,6-hexahydrospiro[pyran-4,3′-pyrrolo[3,2-b]pyridine]-5′-carbonyl)-3,3-dimethylpiperazin-1-yl)-2,4-dimethylnicotinic acid;
4-(1′-(4-chloro-3-fluorophenyl)-1′,2′-dihydrospiro[cyclopropane-1,3′-pyrrolo[3,2-b]pyridine]-5′-carbonyl)-3,3-dimethylpiperazin-2-one;
4-(1′-(4-chloro-3-fluorophenyl)-1′,2′,4,5-tetrahydro-2H-spiro[furan-3,3′-pyrrolo[3,2-b]pyridine]-5′-carbonyl)-3,3-dimethylpiperazin-2-one;
methyl 6-(4-(1′-(4-chloro-3-fluorophenyl)-1′,2′,4,5-tetrahydro-2H-spiro[furan-3,3′-pyrrolo[3,2-b]pyridine]-5′-carbonyl)-3,3-dimethylpiperazin-1-yl)-2,4-dimethylnicotinate;
6-(4-(1′-(4-chloro-3-fluorophenyl)-1′,2′,4,5-tetrahydro-2H-spiro[furan-3,3′-pyrrolo[3,2-b]pyridine]-5′-carbonyl)-3,3-dimethylpiperazin-1-yl)-2,4-dimethylnicotinic acid;
4-(8-(4-chloro-3-fluorophenyl)-3a-methyl-3,3a,8,8a-tetrahydro-2H-furo[3′,2′:4,5]pyrrolo[3,2-b]pyridine-5-carbonyl)-3,3-dimethylpiperazin-2-one;
cis-4-(8-(4-chloro-3-fluorophenyl)-3a-methyl-3,3a,8,8a-tetrahydro-2H-furo[3′,2′:4,5]pyrrolo[3,2-b]pyridine-5-carbonyl)-3,3-dimethylpiperazin-2-one;
4-((3aR,8aR)-8-(4-chloro-3-fluorophenyl)-3a-methyl-3,3a,8,8a-tetrahydro-2H-furo[3′,2′:4,5]pyrrolo[3,2-b]pyridine-5-carbonyl)-3,3-dimethylpiperazin-2-one;
4-((3aS,8aS)-8-(4-chloro-3-fluorophenyl)-3a-methyl-3,3a,8,8a-tetrahydro-2H-furo[3′,2′:4,5]pyrrolo[3,2-b]pyridine-5-carbonyl)-3,3-dimethylpiperazin-2-one;
methyl 2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)acetate;
2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)acetic acid;
ethyl 5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-1,3,4-thiadiazole-2-carboxylate;
(1-(4-chloro-3-fluorophenyl)-3,3-dimethyl-2,3-dihydro-1H-pyrrolo[3,2-b]pyridin-5-yl)(2,2-dimethyl-4-(1,3,4-thiadiazol-2-yl)piperazin-1-yl)methanone;
ethyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylthiazole-5-carboxylate;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylthiazole-5-carboxylic acid;
ethyl 2-(2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylthiazol-5-yl)acetate;
2-(2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-methylthiazol-5-yl)acetic acid;
methyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-5-methylthiazole-4-carboxylate;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-5-methylthiazole-4-carboxylic acid;
1-(1-(4-chloro-3-fluorophenyl)-3,3-dimethyl-2,3-dihydro-1H-pyrrolo[3,2-b]pyridine-5-carbonyl)-7,7-dimethyl-1,4-diazepan-5-one;
8-(1-(4-chloro-3-fluorophenyl)-3,3-dimethyl-2,3-dihydro-1H-pyrrolo[3,2-b]pyridine-5-carbonyl)-2,8-diazaspiro[4.5]decan-1-one;
8-(1-(4-chloro-3-fluorophenyl)-3,3-dimethyl-2,3-dihydro-1H-pyrrolo[3,2-b]pyridine-5-carbonyl)-2,8-diazaspiro[4.5]decan-3-one;
1-(1-(1-(4-chloro-3-fluorophenyl)-3,3-dimethyl-2,3-dihydro-1H-pyrrolo[3,2-b]pyridine-5-carbonyl)piperidin-4-yl)imidazolidin-2-one;
ethyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-(trifluoromethyl)thiazole-5-carboxylate;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-4-(trifluoromethyl)thiazole-5-carboxylic acid;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-N,N-dimethylthiazole-4-carboxamide;
(1-(4-chloro-3-fluorophenyl)-3,3-dimethyl-2,3-dihydro-1H-pyrrolo[3,2-b]pyridin-5-yl)(4-(4-(4-hydroxypiperidine-1-carbonyl)thiazol-2-yl)-2,2-dimethylpiperazin-1-yl)methanone;
(1-(4-chloro-3-fluorophenyl)-3,3-dimethyl-2,3-dihydro-1H-pyrrolo[3,2-b]pyridin-5-yl)(2,2-dimethyl-4-(4-(morpholine-4-carbonyl)thiazol-2-yl)piperazin-1-yl)methanone;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)thiazole-4-carboxamide;
methyl 6-(4-(1-(3-fluoro-4-methylphenyl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(3-fluoro-4-methylphenyl)-3,3-dimethyl-2,3-dihydro-1H-pyrrolo[3,2-b]pyridine-5-carbonyl)-3,3-dimethylpiperazin-1-yl)-2,4-dimethylnicotinic acid;
methyl 6-(4-(4-(4-chloro-3-fluorophenyl)-2,2-dimethyl-2,3,4,5-tetrahydropyrido[3,2-f][1,4]oxazepine-8-carbonyl)-3,3-dimethylpiperazin-1-yl)-2,4-dimethylnicotinate;
methyl 6-(4-(4-(4-chloro-3-fluorophenyl)-2,2-dimethyl-2,3,4,5-tetrahydropyrido[3,2-f][1,4]oxazepine-8-carbonyl)-3,3-dimethylpiperazin-1-yl)-2,4-dimethylnicotinate;
6-(4-(4-(4-chloro-3-fluorophenyl)-2,2-dimethyl-2,3,4,5-tetrahydropyrido[3,2-f][1,4]oxazepine-8-carbonyl)-3,3-dimethylpiperazin-1-yl)-2,4-dimethylnicotinic acid;
6-(4-(1′-(4-chloro-3-fluorophenyl)-3,3-difluoro-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
6-(4-(1′-(4-chloro-3-fluorophenyl)-3-fluoro-1′,2′-dihydrospiro[cyclobutane-1,3′-pyrrolo[3,2-b]pyridin]-2-en-5′-carbonyl)-3,3-dimethylpiperazin-1-yl)-2,4-dimethylnicotinic acid;
methyl 2-((3S,4S)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetate;
2-((3S,4S)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetic acid;
methyl 2-((3R,4R)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetate;
2-((3R,4R)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetic acid;
ethyl (2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)thiazole-4-carbonyl)glycinate;
(2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)thiazole-4-carbonyl)glycine;
methyl 2-((3S,4R)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetate;
2-((3S,4R)-1-(1-(4-chloro-3-fluorophenyl)-3,3-dimethyl-2,3-dihydro-1H-pyrrolo[3,2-b]pyridine-5-carbonyl)-3-methoxypiperidin-4-yl)acetic acid;
methyl 6-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
(1-(4-chloro-3-fluorophenyl)-3,3-dimethyl-2,3-dihydro-1H-pyrrolo[3,2-b]pyridin-5-yl)(2,2-dimethyl-4-(4-(4-methylpiperazine-1-carbonyl)thiazol-2-yl)piperazin-1-yl)methanone;
methyl 6-(4-(1-(4-chloro-3-fluorophenyl)-3,3-diethyl-2,3-dihydro-1H-pyrrolo[3,2-b]pyridine-5-carbonyl)-3,3-dimethylpiperazin-1-yl)-2,4-dimethylnicotinate;
6-(4-(1-(4-chloro-3-fluorophenyl)-3,3-diethyl-2,3-dihydro-1H-pyrrolo[3,2-b]pyridine-5-carbonyl)-3,3-dimethylpiperazin-1-yl)-2,4-dimethylnicotinic acid;
methyl 2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)isonicotinate;
2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)isonicotinic acid;
ethyl 3-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)isonicotinate;
3-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)isonicotinic acid;
ethyl 6-((1-(1-(4-chloro-3-fluorophenyl)-3,3-dimethyl-2,3-dihydro-1H-pyrrolo[3,2-b]pyridine-5-carbonyl)piperidin-3-yl)amino)nicotinate;
6-((1-(1-(4-chloro-3-fluorophenyl)-3,3-dimethyl-2,3-dihydro-1H-pyrrolo[3,2-b]pyridine-5-carbonyl)piperidin-3-yl)amino)nicotinic acid;
ethyl 6-((1-(1-(4-chloro-3-fluorophenyl)-3,3-dimethyl-2,3-dihydro-1H-pyrrolo[3,2-b]pyridine-5-carbonyl)piperidin-3-yl)(methyl)amino)nicotinate;
6-((1-(1-(4-chloro-3-fluorophenyl)-3,3-dimethyl-2,3-dihydro-1H-pyrrolo[3,2-b]pyridine-5-carbonyl)piperidin-3-yl)(methyl)amino)nicotinic acid hydrochloride;
methyl 6-(4-(1′-(3,4-difluorophenyl)-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
6-(4-(1′-(3,4-difluorophenyl)-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
methyl 2-(2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-4-yl)acetate;
2-(2-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-4-yl)acetic acid;
ethyl 2-(5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-2-yl)acetate;
2-(5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-2-yl)acetic acid;
methyl 2-(6-(4-(1′-(4-chloro-3-fluorophenyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-(1′-(4-chloro-3-fluorophenyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
1′-(1-(4-chloro-3-fluorophenyl)-3,3-dimethyl-2,3-dihydro-1H-pyrrolo[3,2-b]pyridine-5-carbonyl)spiro[indoline-3,3′-pyrrolidin]-2-one;
8-(1-(4-chloro-3-fluorophenyl)-3,3-dimethyl-2,3-dihydro-1H-pyrrolo[3,2-b]pyridine-5-carbonyl)-1-oxa-3,8-diazaspiro[4.5]decan-2-one;
ethyl 6-(6-(1-(4-chloro-3-fluorophenyl)-3,3-dimethyl-2,3-dihydro-1H-pyrrolo[3,2-b]pyridine-5-carbonyl)-2,6-diazaspiro[3.3]heptan-2-yl)nicotinate;
6-(6-(1-(4-chloro-3-fluorophenyl)-3,3-dimethyl-2,3-dihydro-1H-pyrrolo[3,2-b]pyridine-5-carbonyl)-2,6-diazaspiro[3.3]heptan-2-yl)nicotinic acid;
methyl 2-(1-(1′-(4-chloro-3-fluorophenyl)-1′,2′-dihydrospiro[cyclobutane-1,3′-pyrrolo[3,2-b]pyridine]-5′-carbonyl)piperidin-4-yl)acetate;
2-(1-(1′-(4-chloro-3-fluorophenyl)-1′,2′-dihydrospiro[cyclobutane-1,3′-pyrrolo[3,2-b]pyridine]-5′-carbonyl)piperidin-4-yl)acetic acid;
4-(1′-(4-chloro-3-fluorophenyl)-1′,2′-dihydrospiro[cyclobutane-1,3′-pyrrolo[3,2-b]pyridine]-5′-carbonyl)-3,3-dimethylpiperazin-2-one;
methyl 2-(6-(4-(1′-(4-chloro-3-fluorophenyl)-3,3-difluoro-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-(1′-(4-chloro-3-fluorophenyl)-3,3-difluoro-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
ethyl 2-((1R,5S)-8-(1-(4-chloro-3-fluorophenyl)-3,3-dimethyl-2,3-dihydro-1H-pyrrolo[3,2-b]pyridine-5-carbonyl)-8-azabicyclo[3.2.1]octan-3-yl)acetate;
2-((1R,5S)-8-(1-(4-chloro-3-fluorophenyl)-3,3-dimethyl-2,3-dihydro-1H-pyrrolo[3,2-b]pyridine-5-carbonyl)-8-azabicyclo[3.2.1]octan-3-yl)acetic acid;
methyl 6-(4-(6-(5-(4-(5-(methoxycarbonyl)-4,6-dimethylpyridin-2-yl)-2,2-dimethylpiperazine-1-carbonyl)-3,3-dimethyl-2,3-dihydro-1H-pyrrolo[3,2-b]pyridin-1-yl)nicotinoyl)-3,3-dimethylpiperazin-1-yl)-2,4-dimethylnicotinate;
methyl 6-(4-(1-(5-chloropyridin-2-yl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(6-(5-(4-(5-carboxy-4,6-dimethylpyridin-2-yl)-2,2-dimethylpiperazine-1-carbonyl)-3,3-dimethyl-2,3-dihydro-1H-pyrrolo[3,2-b]pyridin-1-yl)nicotinoyl)-3,3-dimethylpiperazin-1-yl)-2,4-dimethylnicotinic acid;
6-(4-(1-(5-chloropyridin-2-yl)-3,3-dimethyl-2,3-dihydro-1H-pyrrolo[3,2-b]pyridine-5-carbonyl)-3,3-dimethylpiperazin-1-yl)-2,4-dimethylnicotinic acid;
methyl 6-(4-(3,3-dimethyl-1-(pyridin-2-yl)-2,3-dihydro-1H-pyrrolo[3,2-b]pyridine-5-carbonyl)-3,3-dimethylpiperazin-1-yl)-2,4-dimethylnicotinate;
ethyl 1-(1-(4-chloro-3-fluorophenyl)-3,3-dimethyl-2,3-dihydro-1H-pyrrolo[3,2-b]pyridine-5-carbonyl)-4-(pyridin-2-yl)piperidine-3-carboxylate;
(cis)-1-(1-(4-chloro-3-fluorophenyl)-3,3-dimethyl-2,3-dihydro-1H-pyrrolo[3,2-b]pyridine-5-carbonyl)-4-(pyridin-2-yl)piperidine-3-carboxylic acid;
(trans)-1-(1-(4-chloro-3-fluorophenyl)-3,3-dimethyl-2,3-dihydro-1H-pyrrolo[3,2-b]pyridine-5-carbonyl)-4-(pyridin-2-yl)piperidine-3-carboxylic acid;
methyl 6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-3-methylpicolinate;
6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-3-methylpicolinic acid;
methyl 5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-2-methoxynicotinate;
5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)-2-methoxynicotinic acid;
methyl 6-(4-((1S,3R)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
methyl 6-(4-((1R,3R)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
methyl 6-(4-((3R)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
6-(4-((1S,3R)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
6-(4-((1R,3R)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
6-(4-((3R)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
methyl 6-(4-((1S,3S)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
methyl 6-(4-((1R,3S)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
methyl 6-(4-((3S)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinate;
6-(4-((1S,3S)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
6-(4-((1R,3S)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
6-(4-((3S)-1′-(4-chloro-3-fluorophenyl)-3-methoxy-1′,2′-dihydrospiro[cyclopentane-1,3′-pyrrolo[3,2-b]pyridine]-5′-carbonyl)-3,3-dimethylpiperazin-1-yl)-2,4-dimethylnicotinic acid;
methyl 2-(6-(4-(1-(4-chloro-3-fluorophenyl)-3,3-diethyl-2,3-dihydro-1H-pyrrolo[3,2-b]pyridine-5-carbonyl)-3,3-dimethylpiperazin-1-yl)pyridin-3-yl)acetate;
2-(6-(4-(1-(4-chloro-3-fluorophenyl)-3,3-diethyl-2,3-dihydro-1H-pyrrolo[3,2-b]pyridine-5-carbonyl)-3,3-dimethylpiperazin-1-yl)pyridin-3-yl)acetic acid;
methyl 5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)nicotinate;
methyl 5-(4-(1-(3-fluoro-4-(4-(5-(methoxycarbonyl)pyridin-3-yl)-2,2-dimethylpiperazine-1-carbonyl)phenyl)-3,3-dimethyl-2,3-dihydro-1H-pyrrolo[3,2-b]pyridine-5-carbonyl)-3,3-dimethylpiperazin-1-yl)nicotinate;
5-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)nicotinic acid;
5-(4-(4-(5-(4-(5-carboxypyridin-3-yl)-2,2-dimethylpiperazine-1-carbonyl)-3,3-dimethyl-2,3-dihydro-1H-pyrrolo[3,2-b]pyridin-1-yl)-2-fluorobenzoyl)-3,3-dimethylpiperazin-1-yl)nicotinic acid;
methyl 2-(6-(4-((1s,3s)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-((1s,3s)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
methyl 6-(4-((1s,3s)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-((1s,3s)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 2-(6-(4-((1r,3r)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-((1r,3r)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
methyl 6-(4-((1r,3r)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-((1r,3r)-1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 2-(6-(4-(1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-(1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
methyl 6-(4-(1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-(1′-(4-chloro-3-fluorophenyl)-3-methyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)propanoate;
2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)propanoic acid;
methyl 6-(4-(1-(4-cyanocyclohexyl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(4-carbamoylcyclohexyl)-3,3-dimethyl-2,3-dihydro-1H-pyrrolo[3,2-b]pyridine-5-carbonyl)-3,3-dimethylpiperazin-1-yl)-2,4-dimethylnicotinic acid;
methyl 2-(6-(4-(1-(4,4-difluorocyclohexyl)-3,3-dimethyl-2,3-dihydro-1H-pyrrolo[3,2-b]pyridine-5-carbonyl)-3,3-dimethylpiperazin-1-yl)pyridin-3-yl)acetate;
2-(6-(4-(1-(4,4-difluorocyclohexyl)-3,3-dimethyl-2,3-dihydro-1H-pyrrolo[3,2-b]pyridine-5-carbonyl)-3,3-dimethylpiperazin-1-yl)pyridin-3-yl)acetic acid;
4-(5-(4-(4,6-dimethylpyridin-2-yl)-2,2-dimethylpiperazine-1-carbonyl)-3,3-dimethyl-2,3-dihydro-1H-pyrrolo[3,2-b]pyridin-1-yl)cyclohexane-1-carbonitrile;
methyl 2-(6-(4-(1-cyclopentyl-3,3-dimethyl-2,3-dihydro-1H-pyrrolo[3,2-b]pyridine-5-carbonyl)-3,3-dimethylpiperazin-1-yl)pyridin-3-yl)acetate;
2-(6-(4-(1-cyclopentyl-3,3-dimethyl-2,3-dihydro-1H-pyrrolo[3,2-b]pyridine-5-carbonyl)-3,3-dimethylpiperazin-1-yl)pyridin-3-yl)acetic acid;
methyl 2-(6-(4-(1-(4-cyanocyclohexyl)-3,3-dimethyl-2,3-dihydro-1H-pyrrolo[3,2-b]pyridine-5-carbonyl)-3,3-dimethylpiperazin-1-yl)pyridin-3-yl)acetate;
2-(6-(4-(1-(4-cyanocyclohexyl)-3,3-dimethyl-2,3-dihydro-1H-pyrrolo[3,2-b]pyridine-5-carbonyl)-3,3-dimethylpiperazin-1-yl)pyridin-3-yl)acetic acid;
methyl 2-(6-(4-(5-(3,4-difluorophenyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(3,4-difluorophenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 2-(6-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)-2-methylpropanoate;
2-(6-(4-(1-(4-chloro-3-fluorophenyl)-3,3-dimethyl-2,3-dihydro-1H-pyrrolo[3,2-b]pyridine-5-carbonyl)-3,3-dimethylpiperazin-1-yl)pyridin-3-yl)-2-methylpropanoic acid;
methyl 2-(6-(4-(5-(4-chloro-3-fluorophenyl)-8,8-dimethyl-5,6,7,8-tetrahydro-1,5-naphthyridine-2-carbonyl)-3,3-dimethylpiperazin-1-yl)pyridin-3-yl)acetate;
2-(6-(4-(5-(4-chloro-3-fluorophenyl)-8,8-dimethyl-5,6,7,8-tetrahydro-1,5-naphthyridine-2-carbonyl)-3,3-dimethylpiperazin-1-yl)pyridin-3-yl)acetic acid;
methyl 2-(6-(4-(4-(4-chloro-3-fluorophenyl)-7,7-dimethyl-4,5,6,7-tetrahydropyrazolo[1,5-a]pyrimidine-2-carbonyl)-3,3-dimethylpiperazin-1-yl)pyridin-3-yl)acetate;
2-(6-(4-(4-(4-chloro-3-fluorophenyl)-7,7-dimethyl-4,5,6,7-tetrahydropyrazolo[1,5-a]pyrimidine-2-carbonyl)-3,3-dimethylpiperazin-1-yl)pyridin-3-yl)acetic acid;
methyl 6-(4-(5-(3,4-difluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(3,4-difluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 6-(4-(5-cyclopentyl-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-cyclopentyl-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 6-(4-(5-(3-fluoro-4-methylphenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(3-fluoro-4-methylphenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 6-(4-(5-(4-chloro-3,5-difluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(4-chloro-3,5-difluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 6-(4-(5-(3-chloro-4-methylphenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(3-chloro-4-methylphenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 6-(4-(5-(3-chloro-4-fluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(3-chloro-4-fluorophenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 6-(4-(5-(3-chloro-4-(trifluoromethyl)phenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(3-chloro-4-(trifluoromethyl)phenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 2-(6-(4-(5-(4-chloro-3-fluorophenyl)-7,7-dimethyl-4,5,6,7-tetrahydropyrazolo[1,5-a]pyrazine-2-carbonyl)-3,3-dimethylpiperazin-1-yl)pyridin-3-yl)acetate;
2-(6-(4-(5-(4-chloro-3-fluorophenyl)-7,7-dimethyl-4,5,6,7-tetrahydropyrazolo[1,5-a]pyrazine-2-carbonyl)-3,3-dimethylpiperazin-1-yl)pyridin-3-yl)acetic acid;
methyl 2-(6-(4-(3-(4-chloro-3-fluorobenzyl)-1-isobutyl-3-methyl-2,3-dihydro-1H-pyrrolo[2,3-b]pyridine-6-carbonyl)-3,3-dimethylpiperazin-1-yl)pyridin-3-yl)acetate;
2-(6-(4-(3-(4-chloro-3-fluorobenzyl)-1-isobutyl-3-methyl-2,3-dihydro-1H-pyrrolo[2,3-b]pyridine-6-carbonyl)-3,3-dimethylpiperazin-1-yl)pyridin-3-yl)acetic acid;
methyl 6-(4-((1s,3s)-1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-((1s,3s)-1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 6-(4-((1r,3r)-1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-((1r,3r)-1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 6-(4-(1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-(1′-(4-chloro-3-fluorophenyl)-3-methoxy-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 2-(6-(4-(5-(3-fluoro-4-methylphenyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(3-fluoro-4-methylphenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 2-(6-(4-(5-(3-chloro-4-fluorophenyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(3-chloro-4-fluorophenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 6-(4-(5′-(4-chloro-3-fluorophenyl)-5′,6′-dihydrospiro[cyclopentane-1,7′-pyrrolo[2,3-b]pyrazine]-2′-carbonyl)-3,3-dimethylpiperazin-1-yl)-2,4-dimethylnicotinate;
6-(4-(5′-(4-chloro-3-fluorophenyl)-5′,6′-dihydrospiro[cyclopentane-1,7′-pyrrolo[2,3-b]pyrazine]-2′-carbonyl)-3,3-dimethylpiperazin-1-yl)-2,4-dimethylnicotinic acid;
methyl 6-(4-(1-acetyl-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(1,3-dihydroisobenzofuran-5-yl)-3,3-dimethyl-2,3-dihydro-1H-pyrrolo[3,2-b]pyridine-5-carbonyl)-3,3-dimethylpiperazin-1-yl)-2,4-dimethylnicotinic acid;
methyl 2-(6-(4-(5-(4-fluorophenethyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(4-fluorophenethyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 6-(4-(5-(4-fluorophenethyl)-7,7-dimethyl-6,7-dihydro-5H-pyrrolo[2,3-b]pyrazine-2-carbonyl)-3,3-dimethylpiperazin-1-yl)-2,4-dimethylnicotinate;
2-(6-(4-(5-(4-fluorophenethyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 6-(4-(1′-(4-chloro-3-fluorophenyl)-3,3-dimethyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-(1′-(4-chloro-3-fluorophenyl)-3,3-dimethyl-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 2-(6-(4-(7,7-dimethyl-5-(3,4,5-trifluorophenyl)-6,7-dihydro-5H-pyrrolo[2,3-b]pyrazine-2-carbonyl)-3,3-dimethylpiperazin-1-yl)pyridin-3-yl)acetate;
2-(6-(4-(7,7-dimethyl-5-(3,4,5-trifluorophenyl)-6,7-dihydro-5H-pyrrolo[2,3-b]pyrazine-2-carbonyl)-3,3-dimethylpiperazin-1-yl)pyridin-3-yl)acetic acid;
methyl 2-(6-(4-(5-(3-chloro-4-(trifluoromethyl)phenyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(3-chloro-4-(trifluoromethyl)phenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 2-(6-(4-(5-(3,4-dichlorophenyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(3,4-dichlorophenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 6-(4-(5′-(3,4-difluorophenyl)-5′,6′-dihydrospiro[cyclopentane-1,7′-pyrrolo[2,3-b]pyrazine]-2′-carbonyl)-3,3-dimethylpiperazin-1-yl)-2,4-dimethylnicotinate;
6-(4-(5′-(3,4-difluorophenyl)-5′,6′-dihydrospiro[cyclopentane-1,7′-pyrrolo[2,3-b]pyrazine]-2′-carbonyl)-3,3-dimethylpiperazin-1-yl)-2,4-dimethylnicotinic acid;
methyl 6-(4-((1r,3r)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-((1r,3r)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 6-(4-((1s,3s)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-((1s,3s)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 6-(4-(1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinate;
6-(4-(1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)-2,4-dimethylnicotinic acid;
methyl 2-(6-(4-(5′-(3,4-difluorophenyl)-5′,6′-dihydrospiro[cyclopentane-1,7′-pyrrolo[2,3-b]pyrazine]-2′-carbonyl)-3,3-dimethylpiperazin-1-yl)pyridin-3-yl)acetate;
2-(6-(4-(5′-(3,4-difluorophenyl)-5′,6′-dihydrospiro[cyclopentane-1,7′-pyrrolo[2,3-b]pyrazine]-2′-carbonyl)-3,3-dimethylpiperazin-1-yl)pyridin-3-yl)acetic acid;
methyl 2-(6-(4-(5′-(4-chloro-3-fluorophenyl)-5′,6′-dihydrospiro[cyclobutane-1,7′-pyrrolo[2,3-b]pyrazine]-2′-carbonyl)-3,3-dimethylpiperazin-1-yl)pyridin-3-yl)acetate;
2-(6-(4-(5′-(4-chloro-3-fluorophenyl)-5′,6′-dihydrospiro[cyclobutane-1,7′-pyrrolo[2,3-b]pyrazine]-2′-carbonyl)-3,3-dimethylpiperazin-1-yl)pyridin-3-yl)acetic acid;
methyl 6-(4-(5′-(3,4-difluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-2,4-dimethylnicotinate;
6-(4-(5′-(3,4-difluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-2,4-dimethylnicotinic acid;
methyl 6-(4-(3,3-dimethyl-5′-(3,4,5-trifluorophenyl)-5′,6′-dihydrospiro[cyclobutane-1,7′-pyrrolo[2,3-b]pyrazine]-2′-carbonyl)-3,3-dimethylpiperazin-1-yl)-2,4-dimethylnicotinate;
6-(4-(3,3-dimethyl-5′-(3,4,5-trifluorophenyl)-5′,6′-dihydrospiro[cyclobutane-1,7′-pyrrolo[2,3-b]pyrazine]-2′-carbonyl)-3,3-dimethylpiperazin-1-yl)-2,4-dimethylnicotinic acid;
methyl 6-(4-(5′-(4-chloro-3-fluorophenyl)-5′,6′-dihydrospiro[cyclobutane-1,7′-pyrrolo[2,3-b]pyrazine]-2′-carbonyl)-3,3-dimethylpiperazin-1-yl)-2,4-dimethylnicotinate;
6-(4-(5′-(4-chloro-3-fluorophenyl)-5′,6′-dihydrospiro[cyclobutane-1,7′-pyrrolo[2,3-b]pyrazine]-2′-carbonyl)-3,3-dimethylpiperazin-1-yl)-2,4-dimethylnicotinic acid;
methyl 2-(6-(4-((1s,3s)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-((1s,3s)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
methyl 2-(6-(4-((1r,3r)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-((1r,3r)-1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
methyl 2-(6-(4-(1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetate;
2-(6-(4-(1′-(4-chloro-3-fluorophenyl)-3-(methoxymethyl)-1′,2′-dihydrospiro[cyclobutane-1,3′-pyrrolo[3,2-b]pyridine]-5′-carbonyl)-3,3-dimethylpiperazin-1-yl)pyridin-3-yl)acetic acid;
methyl 2-(6-(4-(5-(4-chloro-3,5-difluorophenyl)-7,7-dimethyl-6,7-dihydro-5H-pyrrolo[2,3-b]pyrazine-2-carbonyl)-3,3-dimethylpiperazin-1-yl)pyridin-3-yl)acetate;
2-(6-(4-(5-(4-chloro-3,5-difluorophenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 2-(6-(4-(5′-(4-chloro-3-fluorophenyl)-5′,6′-dihydrospiro[cyclopentane-1,7′-pyrrolo[2,3-b]pyrazine]-2′-carbonyl)-3,3-dimethylpiperazin-1-yl)pyridin-3-yl)acetate;
2-(6-(4-(5′-(4-chloro-3-fluorophenyl)-5′,6′-dihydrospiro[cyclopentane-1,7′-pyrrolo[2,3-b]pyrazine]-2′-carbonyl)-3,3-dimethylpiperazin-1-yl)pyridin-3-yl)acetic acid;
methyl 2-(6-(4-(5′-(3,4-difluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)pyridin-3-yl)acetate;
2-(6-(4-(5′-(3,4-difluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)pyridin-3-yl)acetic acid;
methyl 6-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-2,4-dimethylnicotinate;
6-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-2,4-dimethylnicotinic acid;
methyl 6-(4-(5-(3,4-dichlorophenyl)-7,7-dimethyl-6,7-dihydro-5H-pyrrolo[2,3-b]pyrazine-2-carbonyl)-3,3-dimethylpiperazin-1-yl)-2,4-dimethylnicotinate;
6-(4-(5-(3,4-dichlorophenyl)-7,7-dimethyl-6,7-dihydro-5H-pyrrolo[2,3-b]pyrazine-2-carbonyl)-3,3-dimethylpiperazin-1-yl)-2,4-dimethylnicotinic acid;
methyl 2-(6-(4-(5-(3-chloro-4-methylphenyl)-7,7-dimethyl-6,7-dihydro-5H-pyrrolo[2,3-b]pyrazine-2-carbonyl)-3,3-dimethylpiperazin-1-yl)pyridin-3-yl)acetate 2-(6-(4-(5-(3-chloro-4-methylphenyl)-7,7-dimethyl-6,7-dihydro-5H-pyrrolo[2,3-b]pyrazine-2-carbonyl)-3,3-dimethylpiperazin-1-yl)pyridin-3-yl)acetic acid;
methyl 2-(6-(4-(5-(4-chloro-3-fluorophenyl)-8-methoxy-7,7-dimethyl-5,6,7,8-tetrahydro-1,5-naphthyridine-2-carbonyl)-3,3-dimethylpiperazin-1-yl)pyridin-3-yl)acetate;
2-(6-(4-(5-(4-chloro-3-fluorophenyl)-8-methoxy-7,7-dimethyl-5,6,7,8-tetrahydro-1,5-naphthyridine-2-carbonyl)-3,3-dimethylpiperazin-1-yl)pyridin-3-yl)acetic acid;
methyl 2-(6-(4-(1-(4-chloro-3-fluorophenyl)-6-methoxy-3,3-dimethyl-2,3-dihydro-1H-pyrrolo[3,2-b]pyridine-5-carbonyl)-3,3-dimethylpiperazin-1-yl)pyridin-3-yl)acetate;
2-(6-(4-(1-(4-chloro-3-fluorophenyl)-6-methoxy-3,3-dimethyl-2,3-dihydro-1H-pyrrolo[3,2-b]pyridine-5-carbonyl)-3,3-dimethylpiperazin-1-yl)pyridin-3-yl)acetic acid;
methyl 2-(6-(4-(3-(4-chloro-3-fluorophenyl)-1-isobutyl-3-methyl-2,3-dihydro-1H-pyrrolo[2,3-b]pyridine-6-carbonyl)-3,3-dimethylpiperazin-1-yl)pyridin-3-yl)acetate;
2-(6-(4-(3-(4-chloro-3-fluorophenyl)-1-isobutyl-3-methyl-2,3-dihydro-1H-pyrrolo[2,3-b]pyridine-6-carbonyl)-3,3-dimethylpiperazin-1-yl)pyridin-3-yl)acetic acid;
2-(6-(4-(1-(4-chloro-3-fluorophenyl)-6-hydroxy-3,3-dimethyl-2,3-dihydro-1H-pyrrolo[3,2-b]pyridine-5-carbonyl)-3,3-dimethylpiperazin-1-yl)pyridin-3-yl)acetic acid;
methyl 8-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-8-oxooctanoate;
8-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-8-oxooctanoic acid;
N-(37-(4-(5′-(4-chloro-3-fluorophenyl)-3,3-dimethyl-5′,6′-dihydrospiro[cyclobutane-1,7′-pyrrolo[2,3-b]pyrazine]-2′-carbonyl)-3,3-dimethylpiperazin-1-yl)-3-methyl-4,17,30,37-tetraoxo-7,10,13,20,23,26-hexaoxa-3,16,29-triazaheptatriacontyl)-N-methylpalmitamide;
methyl 4-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)-2,6-dimethylnicotinate;
4-(4-(5-(4-chloro-3-fluorophenyl)-7,7-dimethyl-6,7-dihydro-5H-pyrrolo[2,3-b]pyrazine-2-carbonyl)-3,3-dimethylpiperazin-1-yl)-2,6-dimethylnicotinic acid;
methyl 2-(6-(4-(1-(4-chloro-3-fluorophenyl)-3,3-dimethyl-2,3-dihydro-1H-pyrido[2,3-b][1,4]oxazine-6-carbonyl)-3,3-dimethylpiperazin-1-yl)pyridin-3-yl)acetate;
2-(6-(4-(1-(4-chloro-3-fluorophenyl)-3,3-dimethyl-2,3-dihydro-1H-pyrido[2,3-b][1,4]oxazine-6-carbonyl)-3,3-dimethylpiperazin-1-yl)pyridin-3-yl)acetic acid;
1-(1-((5′-(4-chloro-3-fluorophenyl)-3,3-dimethyl-5′,6′-dihydrospiro[cyclobutane-1,7′-pyrrolo[2,3-b]pyrazin]-2′-yl)sulfonyl)piperidin-4-yl)imidazolidin-2-one;
methyl 2-(6-(4-(4-(4-chloro-3-fluorophenyl)-2,2-dimethyl-3,4-dihydro-2H-pyrazino[2,3-b][1,4]oxazine-7-carbonyl)-3,3-dimethylpiperazin-1-yl)pyridin-3-yl)acetate;
2-(6-(4-(4-(4-chloro-3-fluorophenyl)-2,2-dimethyl-3,4-dihydro-2H-pyrazino[2,3-b][1,4]oxazine-7-carbonyl)-3,3-dimethylpiperazin-1-yl)pyridin-3-yl)acetic acid;
methyl 2-(6-(4-(5-(4-chloro-3-fluorophenyl)-7,7-dimethyl-4,5,6,7-tetrahydrothiazolo[5,4-c]pyridine-2-carbonyl)-3,3-dimethylpiperazin-1-yl)pyridin-3-yl)acetate;
2-(6-(4-(5-(4-chloro-3-fluorophenyl)-7,7-dimethyl-4,5,6,7-tetrahydrothiazolo[5,4-c]pyridine-2-carbonyl)-3,3-dimethylpiperazin-1-yl)pyridin-3-yl)acetic acid;
tert-butyl 2-(4-(5-(2-methoxy-2-oxoethyl)pyridin-2-yl)-2,2-dimethylpiperazine-1-carbonyl)-7,7-dimethyl-2,4,6,7-tetrahydro-5H-pyrazolo[4,3-c]pyridine-5-carboxylate;
methyl 2-(6-(4-(5-(4-chloro-3-fluorophenyl)-7,7-dimethyl-4,5,6,7-tetrahydro-2H-pyrazolo[4,3-c]pyridine-2-carbonyl)-3,3-dimethylpiperazin-1-yl)pyridin-3-yl)acetate;
2-(6-(4-(5-(4-chloro-3-fluorophenyl)-7,7-dimethyl-4,5,6,7-tetrahydro-2H-pyrazolo[4,3-c]pyridine-2-carbonyl)-3,3-dimethylpiperazin-1-yl)pyridin-3-yl)acetic acid;
methyl 6-(4-(1-(3-fluoro-4-(trifluoromethyl)phenyl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(3-fluoro-4-(trifluoromethyl)phenyl)-3,3-dimethyl-2,3-dihydro-1H-pyrrolo[3,2-b]pyridine-5-carbonyl)-3,3-dimethylpiperazin-1-yl)-2,4-dimethylnicotinic acid;
methyl 6-(4-(7,7-dimethyl-5-(3,4,5-trifluorophenyl)-6,7-dihydro-5H-pyrrolo[2,3-b]pyrazine-2-carbonyl)-3,3-dimethylpiperazin-1-yl)-2,4-dimethylnicotinate;
6-(4-(7,7-dimethyl-5-(3,4,5-trifluorophenyl)-6,7-dihydro-5H-pyrrolo[2,3-b]pyrazine-2-carbonyl)-3,3-dimethylpiperazin-1-yl)-2,4-dimethylnicotinic acid;
methyl 6-(4-(1-(4-cyclopropylphenyl)-3,3-dimethyl-2,3-dihydro-1H-pyrrolo[3,2-b]pyridine-5-carbonyl)-3,3-dimethylpiperazin-1-yl)-2,4-dimethylnicotinate;
6-(4-(1-(4-cyclopropylphenyl)-3,3-dimethyl-2,3-dihydro-1H-pyrrolo[3,2-b]pyridine-5-carbonyl)-3,3-dimethylpiperazin-1-yl)-2,4-dimethylnicotinic acid;
methyl 2-(6-(4-(5-(4-chloro-3-fluorophenyl)-3,7,7-trimethyl-4,5,6,7-tetrahydro-3H-imidazo[4,5-c]pyridine-2-carbonyl)-3,3-dimethylpiperazin-1-yl)pyridin-3-yl)acetate;
2-(6-(4-(5-(4-chloro-3-fluorophenyl)-3,7,7-trimethyl-4,5,6,7-tetrahydro-3H-imidazo[4,5-c]pyridine-2-carbonyl)-3,3-dimethylpiperazin-1-yl)pyridin-3-yl)acetic acid;
4-(5′-(3,4-difluorophenyl)-3,3-dimethyl-5′,6′-dihydrospiro[cyclobutane-1,7′-pyrrolo[2,3-b]pyrazine]-2′-carbonyl)-3,3-dimethylpiperazin-2-one;
or a pharmaceutically acceptable salt or solvate of any of the aforementioned compounds.

20. A compound as defined in any one of claims 1 to 19, wherein said compound is conjugated via a linker to a membrane anchor.

21. A pharmaceutical composition comprising the compound of any one of claims 1 to 20 and a pharmaceutically acceptable excipient.

22. The compound of any one of claims 1 to 20 or the pharmaceutical composition of claim 21 for use in the treatment or prevention of pain, an autoimmune disorder, an autoinflammatory disorder, an inflammatory disorder, a central nervous system disorder, spinal cord injury, a metabolic disorder, a gastrointestinal disorder, a cardiovascular disorder, a fibrotic disorder, a respiratory disorder, a skin disorder, an allergic disorder, or cancer.

23. The compound of any one of claims 1 to 20 or the pharmaceutical composition of claim 21 for use in the treatment or prevention of neuropathic pain, inflammatory pain, cancer pain, post-operative incision pain, fracture pain, osteoporotic fracture pain, gout joint pain, chronic pain, spinal cord injury, atopic dermatitis, contact dermatitis, dry skin dermatitis, seborrhoeic dermatitis, arthritis, rheumatoid arthritis, osteoarthritis, psoriasis, psoriatic arthritis, multiple sclerosis, non-alcoholic steatohepatitis, obesity, diabetes, adipose inflammation, pancreatitis, metabolic syndrome, PAR-2 associated metabolic dysfunction, periodontitis, gingivitis, inflammatory bowel disease, Crohn's disease, ulcerative colitis, peptic ulcer disease, infectious enteritis, irritable bowel syndrome, atherosclerosis, asthma, interstitial lung disease, pulmonary fibrosis, rheumatoid arthritis-associated interstitial lung disease, liver fibrosis, cystic fibrosis, renal fibrosis, peritoneal fibrosis, pancreatic fibrosis, intestinal fibrosis, cardiac fibrosis, skin fibrosis, systemic lupus erythematosus, scleroderma, skin eczema, acne, rosacea, post-inflammatory hyperpigmentation, lichen planus, pruritus, polymyositis, vasculitis, Wegener's granulomatosis, Netherton syndrome, dermatomyositis, uveitis, liver cirrhosis, Alzheimer's disease, Parkinson's disease, dust mite allergy, cockroach allergy, or allergic asthma.

24. The compound of any one of claims 1 to 20 or the pharmaceutical composition of claim 21 for use in the treatment or prevention of cancer.

25. The compound for use according to claim 24 or the pharmaceutical composition for use according to claim 24, wherein said cancer is selected from colorectal cancer, colon cancer, gastric cancer, rectal cancer, liver cancer, breast cancer, pancreatic cancer, cervical cancer, prostate cancer, ovarian cancer, endometrial cancer, uterine sarcoma, germ cell cancer, esophageal cancer, leukemia, lung cancer, adrenal gland cancer, bile duct cancer, bladder cancer, head and neck cancer, kidney cancer, lymphoma, mesothelioma, sarcoma, melanoma, thyroid carcinoma, thymus cancer, and glioblastoma.

26. The compound for use according to claim 24 or 25 or the pharmaceutical composition for use according to claim 24 or 25, wherein said compound or said pharmaceutical composition is to be administered in combination with one or more anticancer drugs.

27. An anticancer drug for use in the treatment or prevention of cancer, wherein said anticancer drug is to be administered in combination with the compound of any one of claims 1 to 20 or the pharmaceutical composition of claim 21.

28. The compound for use according to claim 26 or the pharmaceutical composition for use according to claim 26 or the anticancer drug for use according to claim 27, wherein said anticancer drug(s) is/are selected from immune checkpoint inhibitors.

29. The compound for use according to claim 28 or the pharmaceutical composition for use according to claim 28 or the anticancer drug for use according to claim 28, wherein said immune checkpoint inhibitors are selected from anti-CTLA-4 antibodies, anti-PD-1 antibodies, anti-PD-L1 antibodies, anti-TIGIT antibodies, and anti-LAG3 antibodies.

30. The compound for use according to claim 28 or 29 or the pharmaceutical composition for use according to claim 28 or 29 or the anticancer drug for use according to claim 28 or 29, wherein said immune checkpoint inhibitors are selected from ipilimumab, tremelimumab, nivolumab, pembrolizumab, pidilizumab, cemiplimab, dostarlimab, spartalizumab, camrelizumab, sintilimab, tislelizumab, toripalimab, zimberelimab, AMP-224, AMP-514, JTX-4014, INCMGA00012, APE02058, atezolizumab, avelumab, durvalumab, KN035, CK-301, BMS-936559, MEDI4736, MPDL3280A, MDX-1105, MEDI6469, bintrafusp alfa, tiragolumab, vibostolimab, domvanalimab, etigilimab, BMS-986207, EOS-448, COM902, ASP8374, SEA-TGT, BGB-A1217, IBI-939, M6223, relatlimab, ieramilimab, encelimab, tebotelimab, REGN3767, FS118, IMP701, and IMP731.

31. In vitro use of a compound as defined in any one of claims 1 to 20 as a PAR-2 inhibitor.
